(19) United States
(12) Patent Application Publication
(10) Pub. No.: US 2023/0089270 A1
(43) Pub. Date: Mar. 23, 2023

(54) DUAL APPLICATION NANOPARTICLE COATED FILTRATION SYSTEM FOR AIR POLLUTION AND VIRUS ABATEMENT

(71) Applicant: Ishika Nag, Oviedo, FL (US)

(72) Inventor: Ishika Nag, Oviedo, FL (US)

(21) Appl. No.: 17/404,513

(22) Filed: Aug. 17, 2021

(51) Int. Cl. A61L 2/232; A61L 2/08; A01N 59/20; A01N 59/16; A01P 1/00; A01N 59/00; A41D 13/11; A62B 23/02; B01D 39/16; B01D 46/00; B32B 5/02; B32B 5/26

(52) U.S. Cl. CPC A61L 2/232; A61L 2/088; A01N 59/20; A01N 59/16; A01P 1/00; A01N 59/00; A41D 13/1192; A62B 23/025; B01D 39/1623; B01D 46/0028; B01D 46/0032; B01D 46/0036; B32B 5/022; B32B 5/269; A61L 2202/26; A61L 2101/30

(57) ABSTRACT

A combination of nanoparticles applied onto a mask filtration system, exemplarily including 1%-10% graphene; 1%-10% titanium dioxide; 1%-10% zinc oxide; 1%-10% copper oxide; 1%-10% silicon dioxide; and balance solvent is provided.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM EFS-WEB

Not Applicable

### STATEMENT REGARDING PRIOR DISCLOSURES BY THE INVENTOR OR A JOINT INVENTOR

Not Applicable

### BACKGROUND OF THE INVENTION

The majority (about 91%) of the world's population lives in places exceeding World Health Organization's air quality guidelines and 7 million people die every year because of air pollution. The primary causes of such premature deaths are chronic obstructive pulmonary disease, heart disease, stroke, lung cancer and acute respiratory infections in children. PM_{2.5}, due to its small size, is capable of penetrating deep into lung passageways and entering the bloodstream causing cardio-vascular, cerebro-vascular and respiratory impacts. Furthermore, long term exposure to air pollution has been found to increase the vulnerability to the most severe impacts of coronavirus outbreaks such as SARS in 2003 and COVID-19 in 2020. An increase of only 1 μg/m³ in PM_{2.5} is associated with an 11% increase in the COVID-19 death rate in the United States.

Abatement technologies such as ionic and High Efficiency Particulate Air (HEPA) air filtration systems have been developed that can filter PM_{2.5} particles significantly but remain quite expensive and unaffordable to communities with limited resources. Therefore, a cost-effective and efficient abatement system is essential in helping to resolve the issue.

### FIELD OF THE INVENTION

The invention is directed generally to air pollution prevention and viral particle abatement in filtration systems and more particularly to a system of specific nanoparticles applied to a commonly available filter or face mask and enhancing its particle and virus filtration capabilities.

### SUMMARY OF THE INVENTION

An objective of this invention are to improve the Particle Filtration Efficiency (PFE) and the Virus Filtration Efficiency (VFE) of a regular mask by the impregnation of an optimized mixture of nanomaterials, while demonstrating that the Nanoparticle Retention Efficacy (NRE) are within acceptable Permissible Exposure Limits (PEL), as defined by the Occupational Safety and Health Administration (OSHA).

Furthermore, a goal of this invention is to also develop a simple application technique of the nanoparticles such that it can be easily applied to various filtration systems in different parts of the world, thus providing an affordable alternative to expensive high quality air filtration devices with comparable air filtration capabilities.

Another objective of this invention is to provide a combination of nanoparticles applied onto a filtration system, exemplarily including 0%-10% graphene; 0%-10% titanium dioxide; 0%-10% zinc oxide; 0%-10% copper oxide; 0%-10% silicon dioxide; and balance solvent.

### BRIEF DESCRIPTION OF THE DRAWINGS

FIG. 1A is a schematic of an embodiment of the deposition of nanoparticles on a multi-layer filtration system in a typical ‘surgical mask’.

FIG. 1B is a schematic of an embodiment of the nanoparticles being deposited on the outer layer of the mask.

FIG. 1C is a schematic of an embodiment of the nanoparticles being deposited on the middle layer of the mask.

FIG. 2A is a schematic of an embodiment of the oxidation and adsorption mechanisms of PM_{2.5} particles by the nanoparticle mixture that is impregnated onto the layers of the mask.

FIG. 2B is a schematic of an embodiment of the oxidation mechanism of PM_{2.5} particles with the embedded nanoparticles.

FIG. 2C is a schematic of an embodiment of the adsorption mechanism of PM_{2.5} particles with the embedded nanoparticles.

### DETAILED DESCRIPTION

Surgical masks are typically made of 3 ply non-woven polypropylene. The outer non-woven layer-blocks big droplets and particles, the middle melt-blown layer is for electrostatic filtration while the inner non-woven layer—absorbs breathed vapor. N95 and KN95 masks are made of 4-5 layers of non-woven fabric often made of polypropylene. The two outward protective layers are created by ‘spun bonding’ (nozzle blowing melted threads on a thermoplastic polymer) for protection against the outside environment. The stiff pre-filtration layers between the spun bond layers help mold and shape the mask. There is another layer of melt-blown electret/polarized non-woven material which determines filtration efficiency.

A particulate air filter is a device composed of fibrous or porous materials which removes solid particulates such as dust, pollen, mold, and bacteria from the air. Filters containing an adsorbent or catalyst such as charcoal (carbon) may also remove odors and gaseous pollutants such as volatile organic compounds or ozone. Air filters are used in applications where air quality is important, notably in building ventilation systems and in engines.

Nanoparticles possess at least one dimension of 1 to 100 nanometers (nm; 1 nm=10⁻⁹ meter). Particles have diameter less than 100 nm exhibit new size-dependent properties compared with the bulk material. Nanoparticles exist in the natural world and are also created from human activities. For example, there are several types of engineered nanomaterials such as carbon nanotubes, nanocomposites, quantum dots, fullerenes, quantum wires, and nanofibers Because of the submicroscopic size of nanomaterials, nanomaterials have unique material characteristics, and manufactured nanoparticles may find practical applications in a variety of areas, including medicine, engineering, catalysis, and environmental remediation.

Nanoparticles have a high surface to volume ratio, which enhances the entrapment of particulate matter by adsorption. Some of the nanoparticles used for this experiment like graphene, titanium dioxide (TiO₂), and zinc oxide (ZnO), are known to have filtration properties due to their high adsorption capabilities. TiO₂ nanoparticles, with its photocatalytic properties, absorb the ultraviolet component of sunlight and act as a catalyst to form reactive hydroxyl (•OH) radicals and the superoxide anion (O2•-) from atmospheric moisture and oxygen. These radicals react with the PM_{2.5} particles due to their strong oxidizing capabilities converting them into CO₂ and H₂O.

Metal based nanoparticles, like Copper Oxide and Zinc Oxide, have unique physico-chemical properties due to their small size and high specific surface area, which enable them to interact with viruses. Respiratory diseases such as COPD, bronchitis, and asthma lead to the overexpression of ACE-2 enzyme in human respiratory cells for viral attachment. The SARS-CoV-2 virus primarily attacks the respiratory tract 35. Its spike protein attaches to the over expressed ACE-2 receptors in the epithelial cells of the tract, thus causing respiratory disorders leading to fatalities. Metal-based nanoparticles generate Reactive Oxygen Species (ROS) which oxidize viral proteins and nucleic acids, such as the spike protein in SARS-COV2 virus. Other similar viruses include MERS, SARS, Influenza, and the like.

The combination of nanoparticles specifically researched and selected for the purposes of this invention have filtration and biocidal capabilities, as well as the safety of nanoparticle usage being of significant importance and continues to be a subject of research worldwide. Hence, certain nanoparticles (e.g., silver) known to have toxic properties were avoided since there is a risk of inhalation of these nanoparticles. Nanoparticles researched and selected for this invention are known for their clinical safety and non-toxicity and are used in cosmetic and biomedical applications (e.g., pill coatings, sunscreens). The risk of nanoparticles being dislodged from the mask and inhaled were evaluated as per OSHA standards. The spray deposition method aerosolizes nanoparticles with compressed air and enables them to penetrate the superficial layers of the mask and get embedded in the inner layers, hence having a high retention efficacy. Nanoparticles researched and selected for this invention include, but are not limited to the following:

Graphene is an allotrope of Carbon (one of several atomic structures that Carbon exists in nature—Diamond, Graphite, Charcoal, Graphene, Fullerenes etc.). The graphene allotrope consists of a single layer of carbon atoms arranged in a hexagonal lattice structure. Amongst its many unique properties, it has excellent adsorption characteristics (particles adhere to the surface); and since nanoparticles have a high surface to volume ratio, the adsorption property enhances its ability as a filtration media.

Titanium dioxide (TiO₂) nanoparticles absorb the ultraviolet component of sunlight, acting as a catalyst to form reactive hydroxyl (OH) radicals in the presence of atmospheric moisture. These radicals can oxidize and destroy most pollutant molecules.

Zinc oxide (ZnO) nanoparticle is an inorganic metal oxide which is non-toxic, and can safely be used as medicine, preservative in packaging, and an antimicrobial agent. Zinc oxide is an essential ingredient of many enzymes, sunscreens, and ointments for pain and itch relief. The graphene works well with ZnO/TiO₂ (or any other photocatalytic molecule) as its hexagonal lattice structure acts as a matrix to hold them.

Copper Oxide (CuO) increases the antiviral capability by enhancing the photoactivity by lowering the band-gap of Cu—TiO₂, making the electron jump between bands quicker, especially in the visible light spectrum, since TiO₂ works better in the UV light spectrum. It is better at eliminating bacteriophages and can be used in the dark or with a UV light. The Cu ions catalyze the production of ROS (reactive oxygen species) which degrade the virus's genome. The slow release of Cu ions provides long-lasting virucidal activity.

Silicon Dioxide (SiO₂) exhibits antiviral ability against enveloped virions by attachment inhibition. Due to its large specific area, the viral biocidal property is enhanced when mixed with TiO₂.

The selection, combination and concentration of specific nanoparticle mixtures was determined using a statistical Design of Experiment study, as illustrated in Table 1. After several trials with different nanoparticles and mixture concentrations, the optimum ones were determined based on their particle filtration efficiency. A statistical ANOVA analysis was used to determine the main and interaction effects of the nanoparticles. A Gage R&R study was performed on the experimental set-up to ensure repeatability and reproducibility of the results.

All the nanoparticles used, were in the 40-50 nm size range, but the size could be larger (e.g., 50-100 nm) or smaller (e.g., 1-40 nm). Ethanol was chosen as a solvent since it penetrates the fibrous filtration media in its liquid state, impregnates the nanoparticles in the pores of the filters and then evaporates at room temperature, but other solvents could be used including but not limited to water, methanol, acetone, chloroform, and dichlorobenzene. Different application mechanisms were also tried—pipette, spray bottle and pressurized sprayer system—to determine the most uniform spatial distribution, the best durability and the most nanoparticle retention efficacy (NRE), although other application mechanisms could be used. The pressurized sprayer system which aerosolizes the ethanol-nanoparticle mixture, was found to penetrate the outer layers of the masks and gets the nanoparticles impregnated in the melt-blown middle layer of the masks.

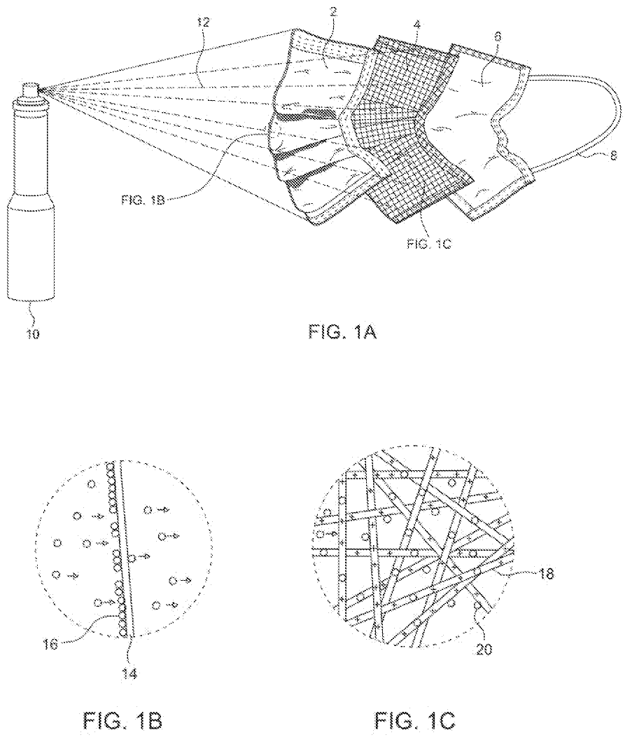

As seen in FIG. 1A, a typical surgical mask comprises of an outer layer 2, a middle layer 4 and an inner layer 6 and a pair of ear loops 8 to hold it in place.

A 1-50%, preferably 1-20%, and most preferably 6-8%, mixture of nanoparticles (weight by weight) was dissolved in the ethanol medium. This solution was then aerosolized using the pressurized sprayer system 10, and the aerosolized spray 12 was directed towards the mask while maintaining a spray-distance of about 6-8 inches. The mask was then air-dried for at least 8 hours and then tested for 160 efficacy.

The outer non-woven layer 14 is deposited with some nanoparticles 16, as illustrated in FIG. 1B while other nanoparticles penetrate onto the melt-blown middle layer 4 with positively charged fibers 18 due to the force of the pressurized sprayer system 10 and attaches onto the fibers of this middle layer 20, as illustrated in FIG. 1C.

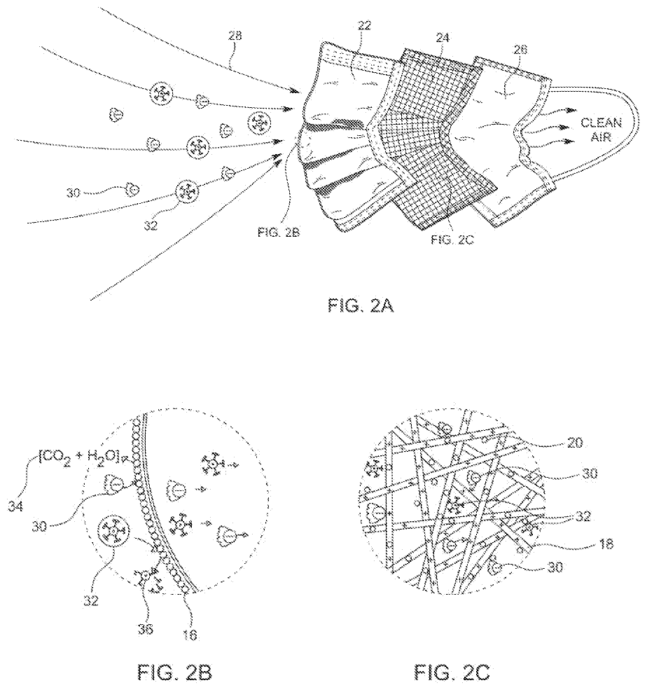

The coated mask now has nanoparticles on its outer layer 22 as well as in its middle layer 24 and some on its inner layer 26 as well, as illustrated in FIG. 2A. The polluted air 28 consists of airborne PM_{2.5} particles 30 and aerosolized virus particles in mucus droplets 32. The embedded nanoparticles 16 oxidize the PM_{2.5} particles 30 as described previously into harmless carbon dioxide and water molecules 34, as illustrated in FIG. 2B. The pollutants that can penetrate the nanoparticle coated 170 outer layer 22 are then adsorbed by the nanoparticles situated in the middle layer 24, as illustrated in FIG. 2C, thus preventing them from going into the human body. The virus particles are similarly captured and oxidized by the metal-based nanoparticles by the generation of Reactive Oxygen Species which oxidize viral proteins and nucleic acids, such as the spike protein in SARS-COV2 virus, thus deactivating the virus particle 36.

### Example I

### Synthesis of Nanoparticle Coatings by Varying Mixture Combinations and Concentrations

Several combinations of nanoparticles and their concentrations are possible within the ranges and were experimented with. A full factorial Design of Experiments (DOE) statistical analysis model, as seen in Table 1, was used to collect and analyze the data to randomize the run order of the experiment, minimize bias and aid with the Analysis of Variance (ANOVA) study. The 1-50%, preferably 4-10%, and most preferably 6-8%, mixture of nanoparticles (weight by weight) researched and selected as being particularly effective for purposes of this invention include without limitation.

### Table 1: Examples of Nanoparticle Combinations and Concentrations

(Detailed Table 1 included, Page 20-89)

Some combinations understood to be particularly effective, include without limitation:
SubExample 1: 8% graphene, 8% titanium dioxide, 8% zinc oxide, 8% copper oxide, 8% silicon dioxide.
SubExample 2: 8% graphene, 8% titanium dioxide, 1% zinc oxide, 8% copper oxide, 1% silicon dioxide.
SubExample 3: 8% graphene, 8% titanium dioxide, 1% zinc oxide, 2% copper oxide, 1% silicon dioxide.
SubExample 4: 1% graphene, 8% titanium dioxide, 8% zinc oxide, 8% copper oxide, 1% silicon dioxide.

### Example II

### Analysis of the Effectiveness of the Nanoparticle Coated Masks for Particle Filtration Efficiency

The coated masks were tested with a wind tunnel specifically designed and created to test for the Particulate Filtration Efficiency (PFE) of the masks, at ambient temperature (25° C.+/−5° C.) conditions. A cardboard box was used as the body of the tunnel. Incense sticks were used as the source of PM_{2.5} and a fan placed inside the tunnel created the draft of PM_{2.5}. Two mannequins, one with a nanoparticle coated mask and another control mannequin without a mask were tested side by side with vacuum pumps (flow rate of 8 l/min) simulating human breathing. Laser particle detectors (Temtop LKC-1000 S) were connected by plastic tubing and funnels to the mannequins and to the vacuum pump to measure PM_{2.5}. An electrical lamp (Hyperikon, 15 W, 5000K) was used to simulate daylight and a UV-A lamp (385-400 nm) was used to activate the photocatalytic properties of the nanoparticles. Two different types of masks— KN95 and Surgical—were tested with nanoparticles, and without any nanoparticles as controls.

The Particulate Filtration Efficiency (PFE) was evaluated (1) by measuring the flow rate (mg/m³) of PM_{2.5}, of the masked mannequin (φ₂) and the control mannequin without mask (φ₁); while both mannequins were placed side-by-side and exposed to the same environment.

$$P F E ( \% ) = \frac{{φ}_{1} - {φ}_{2}}{{φ}_{1}} \times 100 \qquad ( 1 )$$

$${φ}_{1} = {PM}_{2.5} without mask$$

$${φ}_{2} = {PM}_{2.5} with mask$$

### Example III

### Analysis of the Effectiveness of the Nanoparticle Coated Masks for Virus Filtration Efficiency

Virus Filtration Efficiency (VFE) was tested with nebulized NaCl particles (0.5-10 μm diameter) as a surrogate for virus charged respiratory droplets, which are typically 1-5 μm in diameter. A mannequin connected to the nebulizer ‘exhaled’ the NaCl particles which were ‘inhaled’ by the mannequin wearing the nanoparticle coated mask. The mannequins were kept 25 cm apart in a closed chamber to avoid wind draft and tested at ambient temperature (25° C.+/−5° C.). The exposure time was 20 mins per mask to get enough NaCl deposited in the collection chamber to be quantified by the gravimetric measurement procedure.

The Virus Filtration Efficiency (VFE) was calculated (2) by comparing the weight of NaCl deposited on the fine filter paper after being inhaled through the mask (γ₂), to the control case without 225 mask (γ₁).

$$V F E ( \% ) = \frac{{γ}_{2} - {γ}_{2}}{{γ}_{1}} \times 100 \qquad ( 2 )$$

$${γ}_{1} = NaCl ( virus surrogate ) inhaled without mask$$

$${γ}_{2} = NaCl ( virus surrogate ) inhaled with mask$$

### Example IV

### Analysis of the Nanoparticle Coated Masks for Nanoparticle Retention Efficacy

The Nanoparticle Retention Efficacy (NRE) was used to evaluate whether the dislodged nanoparticles from the mask, if inhaled, were still within the Particle Exposure Limits (PEL) as specified by OSHA. Nanoparticle Retention Efficacy (NRE) was tested in a closed chamber to avoid wind draft and tested at ambient temperature (25° C.+/−5° C.). The mannequin wearing the nanoparticle coated mask was connected to a vacuum pump (flow rate 8 l/min) simulating continuous human breathing for an 8-hour normal workday. The dislodged nanoparticles were collected on a fine filter paper (9 nm pore diameter), encased in a collection chamber using two funnels and sealed with a gasket and circumferentially attached clips. A sealing test was performed using the soap bubble leak test.

The PEL Utilization was calculated by comparing the weight of nanoparticles dislodged from the mask during an 8-hour operational period (ω₁) to the PEL limit for that nanoparticle (ω_{ipEL}) and taking a weighted average of the nanoparticles embedded in that mask. The total weight of all the nanoparticles collected in the fine filter paper was measured using the gravimetric method with a micro balance (0.1 mg accuracy), as recommended in the CDC test procedure and the individual weights of 245 the nanoparticles were calculated by the ratio of their molecular weights.

$$P E L Utilization ( \% ) = \frac{1}{n} \times {\sum }_{i = 1}^{n} \frac{{ω}_{i} \div 3.84}{{ω}_{{i}_{PEL}}} \times 100 \qquad ( 3 )$$

$$n = number of N Ps in mask$$

$$i = N P type ( e . g . {TiO}_{2} , ZnO etc . )$$

$${ω}_{i} = weight of N {P}_{i} inhaled from mask [ mg ]$$

$${ω}_{{i}_{PEL}} = Permissible Exposure Limit for N {P}_{i} [ mg / {m}^{3} ]$$

$$\begin{matrix}3.84 = Total volume of air breathed in 8 hours [ {m}^{3} ] \\ = 8 [ l / min ] \times 60 [ min ] \times 8 [ hours ] \times {10}^{- 3} [ {m}^{3} ]\end{matrix}$$

### Example V

### Analysis of the Photocatalytic Activation of Nanoparticles

The photo-catalytic activation of nanoparticles to different kinds of light was tested by simulating daylight with an electrical lamp (Hyperikon, 15 W, 5000K) and a UV-A lamp (385-400 nm) was used to activate the photocatalytic properties of the nanoparticles. A main effects analysis method from the statistical ANOVA analysis method, was used to show that the statistically significant dependence of PFE with light exposure. PFE increased by 7% with exposure to daylight and by 13% in presence of UV light.

### Example VI

### Analysis of the Spatial Uniformity of Nanoparticle Deposition

The deposition method of nanoparticles onto the filtration media were also varied and tested for uniform spatial distribution. Different spray mechanisms were tested using pipettes, spray bottles and pressurized sprayers. The differently sprayed filtration media were tested using the particulate filtration efficiency method to analyze the efficiencies of the deposition methods. The pressurized spray application resulted in the most uniform spatial distribution of the nanoparticles and was chosen as the preferred application method for its simplicity and effectiveness.

### Example VII

### Analysis of the Surface Morphology of Nanoparticle Coatings

The surface morphology of the filters was characterized using the scanning electron 270 microscope (SEM) imaging technique and confirmed the adhesion of the nanoparticles. SEM images enabled the confirmation of the nanoparticle adhesion to the fibrous layers of the masks. The entrapment of particulate matter onto the nanoparticle surfaces were also characterized from the SEM images post filtration experimentation. The embedded nanoparticles enhances the diffusion and electrostatic attraction mechanisms of filtration due to high surface to volume ratio and photocatalytic activation properties.

### Example VIII

### Analysis of the Measurement System Repeatability and Reproducibility

A statistical repeatability and reproducibility study (Gage R&R) was used, using Minitab analytical software, to determine the measurement uncertainty of the experiment. The measurement uncertainty of this experiment was evaluated by repeating the test 10 times (repeatability) each on 3 different coated masks on 2 different days (reproducibility). The tests was repeated for 10 trials each and repeated over different days to measure repeatability and reproducibility. It was seen that 95% of the contribution was from ‘part-to-part variation’ and 5% from the process variation, which is deemed to be an acceptable measurement uncertainty, per statistical guidelines.

As will be understood by those skilled in the art, the selection and concentration of nanoparticles can be further adjusted to optimize durability, uniformity, and retention efficacy. Similarly, the selection and concentration of nanoparticles can be based on filtration efficacy, biocidal capabilities, and clinical safety. Additionally, the deposition method can be adjusted for spatial uniformity, penetration through the outer layers of the filtration layers and retention of the penetrated particles onto the fibrous layers of the filtration systems for continuous and effective operation. Also, the solvent selection can be adjusted, added to, changed, or replaced for desired benefits consistent with the invention.

Although exemplary embodiments of the present combination of nanoparticles applied onto a filtration system has been described in detail, those skilled in the art will understand that various changes, substitutions, variations, and improvements disclosed herein may be made without departing from the spirit and scope of the disclosure in its broadest form.

None of the description in the present application should be read as implying that any 300 particular nanoparticle, element, step, act, or function is an essential element, which must be included in the claim scope: the scope of patented subject matter is defined only by the allowed claims.

TABLE 1
EXAMPLES OF NANOPARTICLE COMBINATIONS
AND CONCENTRATIONS

| | Graphene | ZnO | TiO2 | CuO | SiO2 |
|---|---|---|---|---|---|
| Ex 1 | 0%-1% | 0%-1% | 0%-1% | 0%-1% | 0%-1% |
| Ex 2 | 0%-1% | 0%-1% | 0%-1% | 0%-1% | 1%-2% |
| Ex 3 | 0%-1% | 0%-1% | 0%-1% | 0%-1% | 2%-4% |
| Ex 4 | 0%-1% | 0%-1% | 0%-1% | 0%-1% | 4%-6% |
| Ex 5 | 0%-1% | 0%-1% | 0%-1% | 0%-1% | 6%-8% |
| Ex 6 | 0%-1% | 0%-1% | 0%-1% | 0%-1% | 8%-10% |
| Ex 7 | 0%-1% | 0%-1% | 0%-1% | 1%-2% | 0%-1% |
| Ex 8 | 0%-1% | 0%-1% | 0%-1% | 1%-2% | 1%-2% |
| Ex 9 | 0%-1% | 0%-1% | 0%-1% | 1%-2% | 2%-4% |
| Ex 10 | 0%-1% | 0%-1% | 0%-1% | 1%-2% | 4%-6% |
| Ex 11 | 0%-1% | 0%-1% | 0%-1% | 1%-2% | 6%-8% |
| Ex 12 | 0%-1% | 0%-1% | 0%-1% | 1%-2% | 8%-10% |
| Ex 13 | 0%-1% | 0%-1% | 0%-1% | 2%-4% | 0%-1% |
| Ex 14 | 0%-1% | 0%-1% | 0%-1% | 2%-4% | 1%-2% |
| Ex 15 | 0%-1% | 0%-1% | 0%-1% | 2%-4% | 2%-4% |
| Ex 16 | 0%-1% | 0%-1% | 0%-1% | 2%-4% | 4%-6% |
| Ex 17 | 0%-1% | 0%-1% | 0%-1% | 2%-4% | 6%-8% |
| Ex 18 | 0%-1% | 0%-1% | 0%-1% | 2%-4% | 8%-10% |
| Ex 19 | 0%-1% | 0%-1% | 0%-1% | 4%-6% | 0%-1% |
| Ex 20 | 0%-1% | 0%-1% | 0%-1% | 4%-6% | 1%-2% |
| Ex 21 | 0%-1% | 0%-1% | 0%-1% | 4%-6% | 2%-4% |
| Ex 22 | 0%-1% | 0%-1% | 0%-1% | 4%-6% | 4%-6% |
| Ex 23 | 0%-1% | 0%-1% | 0%-1% | 4%-6% | 6%-8% |
| Ex 24 | 0%-1% | 0%-1% | 0%-1% | 4%-6% | 8%-10% |
| Ex 25 | 0%-1% | 0%-1% | 0%-1% | 6%-8% | 0%-1% |
| Ex 26 | 0%-1% | 0%-1% | 0%-1% | 6%-8% | 1%-2% |
| Ex 27 | 0%-1% | 0%-1% | 0%-1% | 6%-8% | 2%-4% |
| Ex 28 | 0%-1% | 0%-1% | 0%-1% | 6%-8% | 4%-6% |
| Ex 29 | 0%-1% | 0%-1% | 0%-1% | 6%-8% | 6%-8% |
| Ex 30 | 0%-1% | 0%-1% | 0%-1% | 6%-8% | 8%-10% |
| Ex 31 | 0%-1% | 0%-1% | 0%-1% | 8%-10% | 0%-1% |
| Ex 32 | 0%-1% | 0%-1% | 0%-1% | 8%-10% | 1%-2% |
| Ex 33 | 0%-1% | 0%-1% | 0%-1% | 8%-10% | 2%-4% |
| Ex 34 | 0%-1% | 0%-1% | 0%-1% | 8%-10% | 4%-6% |
| Ex 35 | 0%-1% | 0%-1% | 0%-1% | 8%-10% | 6%-8% |
| Ex 36 | 0%-1% | 0%-1% | 0%-1% | 8%-10% | 8%-10% |
| Ex 37 | 0%-1% | 0%-1% | 1%-2% | 0%-1% | 0%-1% |
| Ex 38 | 0%-1% | 0%-1% | 1%-2% | 0%-1% | 1%-2% |
| Ex 39 | 0%-1% | 0%-1% | 1%-2% | 0%-1% | 2%-4% |
| Ex 40 | 0%-1% | 0%-1% | 1%-2% | 0%-1% | 4%-6% |
| Ex 41 | 0%-1% | 0%-1% | 1%-2% | 0%-1% | 6%-8% |
| Ex 42 | 0%-1% | 0%-1% | 1%-2% | 0%-1% | 8%-10% |
| Ex 43 | 0%-1% | 0%-1% | 1%-2% | 1%-2% | 0%-1% |
| Ex 44 | 0%-1% | 0%-1% | 1%-2% | 1%-2% | 1%-2% |
| Ex 45 | 0%-1% | 0%-1% | 1%-2% | 1%-2% | 2%-4% |
| Ex 46 | 0%-1% | 0%-1% | 1%-2% | 1%-2% | 4%-6% |
| Ex 47 | 0%-1% | 0%-1% | 1%-2% | 1%-2% | 6%-8% |
| Ex 48 | 0%-1% | 0%-1% | 1%-2% | 1%-2% | 8%-10% |
| Ex 49 | 0%-1% | 0%-1% | 1%-2% | 2%-4% | 0%-1% |
| Ex 50 | 0%-1% | 0%-1% | 1%-2% | 2%-4% | 1%-2% |
| Ex 51 | 0%-1% | 0%-1% | 1%-2% | 2%-4% | 2%-4% |
| Ex 52 | 0%-1% | 0%-1% | 1%-2% | 2%-4% | 4%-6% |
| Ex 53 | 0%-1% | 0%-1% | 1%-2% | 2%-4% | 6%-8% |
| Ex 54 | 0%-1% | 0%-1% | 1%-2% | 2%-4% | 8%-10% |
| Ex 55 | 0%-1% | 0%-1% | 1%-2% | 4%-6% | 0%-1% |
| Ex 56 | 0%-1% | 0%-1% | 1%-2% | 4%-6% | 1%-2% |
| Ex 57 | 0%-1% | 0%-1% | 1%-2% | 4%-6% | 2%-4% |
| Ex 58 | 0%-1% | 0%-1% | 1%-2% | 4%-6% | 4%-6% |
| Ex 59 | 0%-1% | 0%-1% | 1%-2% | 4%-6% | 6%-8% |
| Ex 60 | 0%-1% | 0%-1% | 1%-2% | 4%-6% | 8%-10% |
| Ex 61 | 0%-1% | 0%-1% | 1%-2% | 6%-8% | 0%-1% |
| Ex 62 | 0%-1% | 0%-1% | 1%-2% | 6%-8% | 1%-2% |
| Ex 63 | 0%-1% | 0%-1% | 1%-2% | 6%-8% | 2%-4% |
| Ex 64 | 0%-1% | 0%-1% | 1%-2% | 6%-8% | 4%-6% |
| Ex 65 | 0%-1% | 0%-1% | 1%-2% | 6%-8% | 6%-8% |
| Ex 66 | 0%-1% | 0%-1% | 1%-2% | 6%-8% | 8%-10% |
| Ex 67 | 0%-1% | 0%-1% | 1%-2% | 8%-10% | 0%-1% |
| Ex 68 | 0%-1% | 0%-1% | 1%-2% | 8%-10% | 1%-2% |
| Ex 69 | 0%-1% | 0%-1% | 1%-2% | 8%-10% | 2%-4% |
| Ex 70 | 0%-1% | 0%-1% | 1%-2% | 8%-10% | 4%-6% |
| Ex 71 | 0%-1% | 0%-1% | 1%-2% | 8%-10% | 6%-8% |
| Ex 72 | 0%-1% | 0%-1% | 1%-2% | 8%-10% | 8%-10% |
| Ex 73 | 0%-1% | 0%-1% | 2%-4% | 0%-1% | 0%-1% |
| Ex 74 | 0%-1% | 0%-1% | 2%-4% | 0%-1% | 1%-2% |
| Ex 75 | 0%-1% | 0%-1% | 2%-4% | 0%-1% | 2%-4% |
| Ex 76 | 0%-1% | 0%-1% | 2%-4% | 0%-1% | 4%-6% |
| Ex 77 | 0%-1% | 0%-1% | 2%-4% | 0%-1% | 6%-8% |
| Ex 78 | 0%-1% | 0%-1% | 2%-4% | 0%-1% | 8%-10% |
| Ex 79 | 0%-1% | 0%-1% | 2%-4% | 1%-2% | 0%-1% |
| Ex 80 | 0%-1% | 0%-1% | 2%-4% | 1%-2% | 1%-2% |
| Ex 81 | 0%-1% | 0%-1% | 2%-4% | 1%-2% | 2%-4% |
| Ex 82 | 0%-1% | 0%-1% | 2%-4% | 1%-2% | 4%-6% |
| Ex 83 | 0%-1% | 0%-1% | 2%-4% | 1%-2% | 6%-8% |
| Ex 84 | 0%-1% | 0%-1% | 2%-4% | 1%-2% | 8%-10% |
| Ex 85 | 0%-1% | 0%-1% | 2%-4% | 2%-4% | 0%-1% |
| Ex 86 | 0%-1% | 0%-1% | 2%-4% | 2%-4% | 1%-2% |
| Ex 87 | 0%-1% | 0%-1% | 2%-4% | 2%-4% | 2%-4% |
| Ex 88 | 0%-1% | 0%-1% | 2%-4% | 2%-4% | 4%-6% |
| Ex 89 | 0%-1% | 0%-1% | 2%-4% | 2%-4% | 6%-8% |
| Ex 90 | 0%-1% | 0%-1% | 2%-4% | 2%-4% | 8%-10% |
| Ex 91 | 0%-1% | 0%-1% | 2%-4% | 4%-6% | 0%-1% |
| Ex 92 | 0%-1% | 0%-1% | 2%-4% | 4%-6% | 1%-2% |
| Ex 93 | 0%-1% | 0%-1% | 2%-4% | 4%-6% | 2%-4% |
| Ex 94 | 0%-1% | 0%-1% | 2%-4% | 4%-6% | 4%-6% |
| Ex 95 | 0%-1% | 0%-1% | 2%-4% | 4%-6% | 6%-8% |
| Ex 96 | 0%-1% | 0%-1% | 2%-4% | 4%-6% | 8%-10% |
| Ex 97 | 0%-1% | 0%-1% | 2%-4% | 6%-8% | 0%-1% |
| Ex 98 | 0%-1% | 0%-1% | 2%-4% | 6%-8% | 1%-2% |
| Ex 99 | 0%-1% | 0%-1% | 2%-4% | 6%-8% | 2%-4% |
| Ex 100 | 0%-1% | 0%-1% | 2%-4% | 6%-8% | 4%-6% |
| Ex 101 | 0%-1% | 0%-1% | 2%-4% | 6%-8% | 6%-8% |
| Ex 102 | 0%-1% | 0%-1% | 2%-4% | 6%-8% | 8%-10% |
| Ex 103 | 0%-1% | 0%-1% | 2%-4% | 8%-10% | 0%-1% |
| Ex 104 | 0%-1% | 0%-1% | 2%-4% | 8%-10% | 1%-2% |
| Ex 105 | 0%-1% | 0%-1% | 2%-4% | 8%-10% | 2%-4% |
| Ex 106 | 0%-1% | 0%-1% | 2%-4% | 8%-10% | 4%-6% |
| Ex 107 | 0%-1% | 0%-1% | 2%-4% | 8%-10% | 6%-8% |
| Ex 108 | 0%-1% | 0%-1% | 2%-4% | 8%-10% | 8%-10% |
| Ex 109 | 0%-1% | 0%-1% | 4%-6% | 0%-1% | 0%-1% |
| Ex 110 | 0%-1% | 0%-1% | 4%-6% | 0%-1% | 1%-2% |
| Ex 111 | 0%-1% | 0%-1% | 4%-6% | 0%-1% | 2%-4% |
| Ex 112 | 0%-1% | 0%-1% | 4%-6% | 0%-1% | 4%-6% |
| Ex 113 | 0%-1% | 0%-1% | 4%-6% | 0%-1% | 6%-8% |
| Ex 114 | 0%-1% | 0%-1% | 4%-6% | 0%-1% | 8%-10% |
| Ex 115 | 0%-1% | 0%-1% | 4%-6% | 1%-2% | 0%-1% |
| Ex 116 | 0%-1% | 0%-1% | 4%-6% | 1%-2% | 1%-2% |
| Ex 117 | 0%-1% | 0%-1% | 4%-6% | 1%-2% | 2%-4% |
| Ex 118 | 0%-1% | 0%-1% | 4%-6% | 1%-2% | 4%-6% |
| Ex 119 | 0%-1% | 0%-1% | 4%-6% | 1%-2% | 6%-8% |
| Ex 120 | 0%-1% | 0%-1% | 4%-6% | 1%-2% | 8%-10% |
| Ex 121 | 0%-1% | 0%-1% | 4%-6% | 2%-4% | 0%-1% |
| Ex 122 | 0%-1% | 0%-1% | 4%-6% | 2%-4% | 1%-2% |
| Ex 123 | 0%-1% | 0%-1% | 4%-6% | 2%-4% | 2%-4% |
| Ex 124 | 0%-1% | 0%-1% | 4%-6% | 2%-4% | 4%-6% |
| Ex 125 | 0%-1% | 0%-1% | 4%-6% | 2%-4% | 6%-8% |
| Ex 126 | 0%-1% | 0%-1% | 4%-6% | 2%-4% | 8%-10% |
| Ex 127 | 0%-1% | 0%-1% | 4%-6% | 4%-6% | 0%-1% |
| Ex 128 | 0%-1% | 0%-1% | 4%-6% | 4%-6% | 1%-2% |
| Ex 129 | 0%-1% | 0%-1% | 4%-6% | 4%-6% | 2%-4% |
| Ex 130 | 0%-1% | 0%-1% | 4%-6% | 4%-6% | 4%-6% |
| Ex 131 | 0%-1% | 0%-1% | 4%-6% | 4%-6% | 6%-8% |
| Ex 132 | 0%-1% | 0%-1% | 4%-6% | 4%-6% | 8%-10% |
| Ex 133 | 0%-1% | 0%-1% | 4%-6% | 6%-8% | 0%-1% |
| Ex 134 | 0%-1% | 0%-1% | 4%-6% | 6%-8% | 1%-2% |
| Ex 135 | 0%-1% | 0%-1% | 4%-6% | 6%-8% | 2%-4% |
| Ex 136 | 0%-1% | 0%-1% | 4%-6% | 6%-8% | 4%-6% |
| Ex 137 | 0%-1% | 0%-1% | 4%-6% | 6%-8% | 6%-8% |
| Ex 138 | 0%-1% | 0%-1% | 4%-6% | 6%-8% | 8%-10% |
| Ex 139 | 0%-1% | 0%-1% | 4%-6% | 8%-10% | 0%-1% |
| Ex 140 | 0%-1% | 0%-1% | 4%-6% | 8%-10% | 1%-2% |
| Ex 141 | 0%-1% | 0%-1% | 4%-6% | 8%-10% | 2%-4% |
| Ex 142 | 0%-1% | 0%-1% | 4%-6% | 8%-10% | 4%-6% |
| Ex 143 | 0%-1% | 0%-1% | 4%-6% | 8%-10% | 6%-8% |
| Ex 144 | 0%-1% | 0%-1% | 4%-6% | 8%-10% | 8%-10% |
| Ex 145 | 0%-1% | 0%-1% | 6%-8% | 0%-1% | 0%-1% |
| Ex 146 | 0%-1% | 0%-1% | 6%-8% | 0%-1% | 1%-2% |
| Ex 147 | 0%-1% | 0%-1% | 6%-8% | 0%-1% | 2%-4% |
| Ex 148 | 0%-1% | 0%-1% | 6%-8% | 0%-1% | 4%-6% |
| Ex 149 | 0%-1% | 0%-1% | 6%-8% | 0%-1% | 6%-8% |
| Ex 150 | 0%-1% | 0%-1% | 6%-8% | 0%-1% | 8%-10% |
| Ex 151 | 0%-1% | 0%-1% | 6%-8% | 1%-2% | 0%-1% |
| Ex 152 | 0%-1% | 0%-1% | 6%-8% | 1%-2% | 1%-2% |
| Ex 153 | 0%-1% | 0%-1% | 6%-8% | 1%-2% | 2%-4% |
| Ex 154 | 0%-1% | 0%-1% | 6%-8% | 1%-2% | 4%-6% |
| Ex 155 | 0%-1% | 0%-1% | 6%-8% | 1%-2% | 6%-8% |
| Ex 156 | 0%-1% | 0%-1% | 6%-8% | 1%-2% | 8%-10% |
| Ex 157 | 0%-1% | 0%-1% | 6%-8% | 2%-4% | 0%-1% |
| Ex 158 | 0%-1% | 0%-1% | 6%-8% | 2%-4% | 1%-2% |
| Ex 159 | 0%-1% | 0%-1% | 6%-8% | 2%-4% | 2%-4% |
| Ex 160 | 0%-1% | 0%-1% | 6%-8% | 2%-4% | 4%-6% |
| Ex 161 | 0%-1% | 0%-1% | 6%-8% | 2%-4% | 6%-8% |
| Ex 162 | 0%-1% | 0%-1% | 6%-8% | 2%-4% | 8%-10% |
| Ex 163 | 0%-1% | 0%-1% | 6%-8% | 4%-6% | 0%-1% |
| Ex 164 | 0%-1% | 0%-1% | 6%-8% | 4%-6% | 1%-2% |
| Ex 165 | 0%-1% | 0%-1% | 6%-8% | 4%-6% | 2%-4% |
| Ex 166 | 0%-1% | 0%-1% | 6%-8% | 4%-6% | 4%-6% |
| Ex 167 | 0%-1% | 0%-1% | 6%-8% | 4%-6% | 6%-8% |
| Ex 168 | 0%-1% | 0%-1% | 6%-8% | 4%-6% | 8%-10% |
| Ex 169 | 0%-1% | 0%-1% | 6%-8% | 6%-8% | 0%-1% |
| Ex 170 | 0%-1% | 0%-1% | 6%-8% | 6%-8% | 1%-2% |
| Ex 171 | 0%-1% | 0%-1% | 6%-8% | 6%-8% | 2%-4% |
| Ex 172 | 0%-1% | 0%-1% | 6%-8% | 6%-8% | 4%-6% |
| Ex 173 | 0%-1% | 0%-1% | 6%-8% | 6%-8% | 6%-8% |
| Ex 174 | 0%-1% | 0%-1% | 6%-8% | 6%-8% | 8%-10% |
| Ex 175 | 0%-1% | 0%-1% | 6%-8% | 8%-10% | 0%-1% |
| Ex 176 | 0%-1% | 0%-1% | 6%-8% | 8%-10% | 1%-2% |
| Ex 177 | 0%-1% | 0%-1% | 6%-8% | 8%-10% | 2%-4% |
| Ex 178 | 0%-1% | 0%-1% | 6%-8% | 8%-10% | 4%-6% |
| Ex 179 | 0%-1% | 0%-1% | 6%-8% | 8%-10% | 6%-8% |
| Ex 180 | 0%-1% | 0%-1% | 6%-8% | 8%-10% | 8%-10% |
| Ex 181 | 0%-1% | 0%-1% | 8%-10% | 0%-1% | 0%-1% |
| Ex 182 | 0%-1% | 0%-1% | 8%-10% | 0%-1% | 1%-2% |
| Ex 183 | 0%-1% | 0%-1% | 8%-10% | 0%-1% | 2%-4% |
| Ex 184 | 0%-1% | 0%-1% | 8%-10% | 0%-1% | 4%-6% |
| Ex 185 | 0%-1% | 0%-1% | 8%-10% | 0%-1% | 6%-8% |
| Ex 186 | 0%-1% | 0%-1% | 8%-10% | 0%-1% | 8%-10% |
| Ex 187 | 0%-1% | 0%-1% | 8%-10% | 1%-2% | 0%-1% |
| Ex 188 | 0%-1% | 0%-1% | 8%-10% | 1%-2% | 1%-2% |
| Ex 189 | 0%-1% | 0%-1% | 8%-10% | 1%-2% | 2%-4% |
| Ex 190 | 0%-1% | 0%-1% | 8%-10% | 1%-2% | 4%-6% |
| Ex 191 | 0%-1% | 0%-1% | 8%-10% | 1%-2% | 6%-8% |
| Ex 192 | 0%-1% | 0%-1% | 8%-10% | 1%-2% | 8%-10% |
| Ex 193 | 0%-1% | 0%-1% | 8%-10% | 2%-4% | 0%-1% |
| Ex 194 | 0%-1% | 0%-1% | 8%-10% | 2%-4% | 1%-2% |
| Ex 195 | 0%-1% | 0%-1% | 8%-10% | 2%-4% | 2%-4% |
| Ex 196 | 0%-1% | 0%-1% | 8%-10% | 2%-4% | 4%-6% |
| Ex 197 | 0%-1% | 0%-1% | 8%-10% | 2%-4% | 6%-8% |
| Ex 198 | 0%-1% | 0%-1% | 8%-10% | 2%-4% | 8%-10% |
| Ex 199 | 0%-1% | 0%-1% | 8%-10% | 4%-6% | 0%-1% |
| Ex 200 | 0%-1% | 0%-1% | 8%-10% | 4%-6% | 1%-2% |
| Ex 201 | 0%-1% | 0%-1% | 8%-10% | 4%-6% | 2%-4% |
| Ex 202 | 0%-1% | 0%-1% | 8%-10% | 4%-6% | 4%-6% |
| Ex 203 | 0%-1% | 0%-1% | 8%-10% | 4%-6% | 6%-8% |
| Ex 204 | 0%-1% | 0%-1% | 8%-10% | 4%-6% | 8%-10% |
| Ex 205 | 0%-1% | 0%-1% | 8%-10% | 6%-8% | 0%-1% |
| Ex 206 | 0%-1% | 0%-1% | 8%-10% | 6%-8% | 1%-2% |
| Ex 207 | 0%-1% | 0%-1% | 8%-10% | 6%-8% | 2%-4% |
| Ex 208 | 0%-1% | 0%-1% | 8%-10% | 6%-8% | 4%-6% |
| Ex 209 | 0%-1% | 0%-1% | 8%-10% | 6%-8% | 6%-8% |
| Ex 210 | 0%-1% | 0%-1% | 8%-10% | 6%-8% | 8%-10% |
| Ex 211 | 0%-1% | 0%-1% | 8%-10% | 8%-10% | 0%-1% |
| Ex 212 | 0%-1% | 0%-1% | 8%-10% | 8%-10% | 1%-2% |
| Ex 213 | 0%-1% | 0%-1% | 8%-10% | 8%-10% | 2%-4% |
| Ex 214 | 0%-1% | 0%-1% | 8%-10% | 8%-10% | 4%-6% |
| Ex 215 | 0%-1% | 0%-1% | 8%-10% | 8%-10% | 6%-8% |
| Ex 216 | 0%-1% | 0%-1% | 8%-10% | 8%-10% | 8%-10% |
| Ex 217 | 0%-1% | 1%-2% | 0%-1% | 0%-1% | 0%-1% |
| Ex 218 | 0%-1% | 1%-2% | 0%-1% | 0%-1% | 1%-2% |
| Ex 219 | 0%-1% | 1%-2% | 0%-1% | 0%-1% | 2%-4% |
| Ex 220 | 0%-1% | 1%-2% | 0%-1% | 0%-1% | 4%-6% |
| Ex 221 | 0%-1% | 1%-2% | 0%-1% | 0%-1% | 6%-8% |
| Ex 222 | 0%-1% | 1%-2% | 0%-1% | 0%-1% | 8%-10% |
| Ex 223 | 0%-1% | 1%-2% | 0%-1% | 1%-2% | 0%-1% |
| Ex 224 | 0%-1% | 1%-2% | 0%-1% | 1%-2% | 1%-2% |
| Ex 225 | 0%-1% | 1%-2% | 0%-1% | 1%-2% | 2%-4% |
| Ex 226 | 0%-1% | 1%-2% | 0%-1% | 1%-2% | 4%-6% |
| Ex 227 | 0%-1% | 1%-2% | 0%-1% | 1%-2% | 6%-8% |
| Ex 228 | 0%-1% | 1%-2% | 0%-1% | 1%-2% | 8%-10% |
| Ex 229 | 0%-1% | 1%-2% | 0%-1% | 2%-4% | 0%-1% |
| Ex 230 | 0%-1% | 1%-2% | 0%-1% | 2%-4% | 1%-2% |
| Ex 231 | 0%-1% | 1%-2% | 0%-1% | 2%-4% | 2%-4% |
| Ex 232 | 0%-1% | 1%-2% | 0%-1% | 2%-4% | 4%-6% |
| Ex 233 | 0%-1% | 1%-2% | 0%-1% | 2%-4% | 6%-8% |
| Ex 234 | 0%-1% | 1%-2% | 0%-1% | 2%-4% | 8%-10% |
| Ex 235 | 0%-1% | 1%-2% | 0%-1% | 4%-6% | 0%-1% |
| Ex 236 | 0%-1% | 1%-2% | 0%-1% | 4%-6% | 1%-2% |
| Ex 237 | 0%-1% | 1%-2% | 0%-1% | 4%-6% | 2%-4% |
| Ex 238 | 0%-1% | 1%-2% | 0%-1% | 4%-6% | 4%-6% |
| Ex 239 | 0%-1% | 1%-2% | 0%-1% | 4%-6% | 6%-8% |
| Ex 240 | 0%-1% | 1%-2% | 0%-1% | 4%-6% | 8%-10% |
| Ex 241 | 0%-1% | 1%-2% | 0%-1% | 6%-8% | 0%-1% |
| Ex 242 | 0%-1% | 1%-2% | 0%-1% | 6%-8% | 1%-2% |
| Ex 243 | 0%-1% | 1%-2% | 0%-1% | 6%-8% | 2%-4% |
| Ex 244 | 0%-1% | 1%-2% | 0%-1% | 6%-8% | 4%-6% |
| Ex 245 | 0%-1% | 1%-2% | 0%-1% | 6%-8% | 6%-8% |
| Ex 246 | 0%-1% | 1%-2% | 0%-1% | 6%-8% | 8%-10% |
| Ex 247 | 0%-1% | 1%-2% | 0%-1% | 8%-10% | 0%-1% |
| Ex 248 | 0%-1% | 1%-2% | 0%-1% | 8%-10% | 1%-2% |
| Ex 249 | 0%-1% | 1%-2% | 0%-1% | 8%-10% | 2%-4% |
| Ex 250 | 0%-1% | 1%-2% | 0%-1% | 8%-10% | 4%-6% |
| Ex 251 | 0%-1% | 1%-2% | 0%-1% | 8%-10% | 6%-8% |
| Ex 252 | 0%-1% | 1%-2% | 0%-1% | 8%-10% | 8%-10% |
| Ex 253 | 0%-1% | 1%-2% | 1%-2% | 0%-1% | 0%-1% |
| Ex 254 | 0%-1% | 1%-2% | 1%-2% | 0%-1% | 1%-2% |
| Ex 255 | 0%-1% | 1%-2% | 1%-2% | 0%-1% | 2%-4% |
| Ex 256 | 0%-1% | 1%-2% | 1%-2% | 0%-1% | 4%-6% |
| Ex 257 | 0%-1% | 1%-2% | 1%-2% | 0%-1% | 6%-8% |
| Ex 258 | 0%-1% | 1%-2% | 1%-2% | 0%-1% | 8%-10% |
| Ex 259 | 0%-1% | 1%-2% | 1%-2% | 1%-2% | 0%-1% |
| Ex 260 | 0%-1% | 1%-2% | 1%-2% | 1%-2% | 1%-2% |
| Ex 261 | 0%-1% | 1%-2% | 1%-2% | 1%-2% | 2%-4% |
| Ex 262 | 0%-1% | 1%-2% | 1%-2% | 1%-2% | 4%-6% |
| Ex 263 | 0%-1% | 1%-2% | 1%-2% | 1%-2% | 6%-8% |
| Ex 264 | 0%-1% | 1%-2% | 1%-2% | 1%-2% | 8%-10% |
| Ex 265 | 0%-1% | 1%-2% | 1%-2% | 2%-4% | 0%-1% |
| Ex 266 | 0%-1% | 1%-2% | 1%-2% | 2%-4% | 1%-2% |
| Ex 267 | 0%-1% | 1%-2% | 1%-2% | 2%-4% | 2%-4% |
| Ex 268 | 0%-1% | 1%-2% | 1%-2% | 2%-4% | 4%-6% |
| Ex 269 | 0%-1% | 1%-2% | 1%-2% | 2%-4% | 6%-8% |
| Ex 270 | 0%-1% | 1%-2% | 1%-2% | 2%-4% | 8%-10% |
| Ex 271 | 0%-1% | 1%-2% | 1%-2% | 4%-6% | 0%-1% |
| Ex 272 | 0%-1% | 1%-2% | 1%-2% | 4%-6% | 1%-2% |
| Ex 273 | 0%-1% | 1%-2% | 1%-2% | 4%-6% | 2%-4% |
| Ex 274 | 0%-1% | 1%-2% | 1%-2% | 4%-6% | 4%-6% |
| Ex 275 | 0%-1% | 1%-2% | 1%-2% | 4%-6% | 6%-8% |
| Ex 276 | 0%-1% | 1%-2% | 1%-2% | 4%-6% | 8%-10% |
| Ex 277 | 0%-1% | 1%-2% | 1%-2% | 6%-8% | 0%-1% |
| Ex 278 | 0%-1% | 1%-2% | 1%-2% | 6%-8% | 1%-2% |
| Ex 279 | 0%-1% | 1%-2% | 1%-2% | 6%-8% | 2%-4% |
| Ex 280 | 0%-1% | 1%-2% | 1%-2% | 6%-8% | 4%-6% |
| Ex 281 | 0%-1% | 1%-2% | 1%-2% | 6%-8% | 6%-8% |
| Ex 282 | 0%-1% | 1%-2% | 1%-2% | 6%-8% | 8%-10% |
| Ex 283 | 0%-1% | 1%-2% | 1%-2% | 8%-10% | 0%-1% |
| Ex 284 | 0%-1% | 1%-2% | 1%-2% | 8%-10% | 1%-2% |
| Ex 285 | 0%-1% | 1%-2% | 1%-2% | 8%-10% | 2%-4% |
| Ex 286 | 0%-1% | 1%-2% | 1%-2% | 8%-10% | 4%-6% |
| Ex 287 | 0%-1% | 1%-2% | 1%-2% | 8%-10% | 6%-8% |
| Ex 288 | 0%-1% | 1%-2% | 1%-2% | 8%-10% | 8%-10% |
| Ex 289 | 0%-1% | 1%-2% | 2%-4% | 0%-1% | 0%-1% |
| Ex 290 | 0%-1% | 1%-2% | 2%-4% | 0%-1% | 1%-2% |
| Ex 291 | 0%-1% | 1%-2% | 2%-4% | 0%-1% | 2%-4% |
| Ex 292 | 0%-1% | 1%-2% | 2%-4% | 0%-1% | 4%-6% |
| Ex 293 | 0%-1% | 1%-2% | 2%-4% | 0%-1% | 6%-8% |
| Ex 294 | 0%-1% | 1%-2% | 2%-4% | 0%-1% | 8%-10% |
| Ex 295 | 0%-1% | 1%-2% | 2%-4% | 1%-2% | 0%-1% |
| Ex 296 | 0%-1% | 1%-2% | 2%-4% | 1%-2% | 1%-2% |
| Ex 297 | 0%-1% | 1%-2% | 2%-4% | 1%-2% | 2%-4% |
| Ex 298 | 0%-1% | 1%-2% | 2%-4% | 1%-2% | 4%-6% |
| Ex 299 | 0%-1% | 1%-2% | 2%-4% | 1%-2% | 6%-8% |
| Ex 300 | 0%-1% | 1%-2% | 2%-4% | 1%-2% | 8%-10% |
| Ex 301 | 0%-1% | 1%-2% | 2%-4% | 2%-4% | 0%-1% |
| Ex 302 | 0%-1% | 1%-2% | 2%-4% | 2%-4% | 1%-2% |
| Ex 303 | 0%-1% | 1%-2% | 2%-4% | 2%-4% | 2%-4% |
| Ex 304 | 0%-1% | 1%-2% | 2%-4% | 2%-4% | 4%-6% |
| Ex 305 | 0%-1% | 1%-2% | 2%-4% | 2%-4% | 6%-8% |
| Ex 306 | 0%-1% | 1%-2% | 2%-4% | 2%-4% | 8%-10% |
| Ex 307 | 0%-1% | 1%-2% | 2%-4% | 4%-6% | 0%-1% |
| Ex 308 | 0%-1% | 1%-2% | 2%-4% | 4%-6% | 1%-2% |
| Ex 309 | 0%-1% | 1%-2% | 2%-4% | 4%-6% | 2%-4% |
| Ex 310 | 0%-1% | 1%-2% | 2%-4% | 4%-6% | 4%-6% |
| Ex 311 | 0%-1% | 1%-2% | 2%-4% | 4%-6% | 6%-8% |
| Ex 312 | 0%-1% | 1%-2% | 2%-4% | 4%-6% | 8%-10% |
| Ex 313 | 0%-1% | 1%-2% | 2%-4% | 6%-8% | 0%-1% |
| Ex 314 | 0%-1% | 1%-2% | 2%-4% | 6%-8% | 1%-2% |
| Ex 315 | 0%-1% | 1%-2% | 2%-4% | 6%-8% | 2%-4% |
| Ex 316 | 0%-1% | 1%-2% | 2%-4% | 6%-8% | 4%-6% |
| Ex 317 | 0%-1% | 1%-2% | 2%-4% | 6%-8% | 6%-8% |
| Ex 318 | 0%-1% | 1%-2% | 2%-4% | 6%-8% | 8%-10% |
| Ex 319 | 0%-1% | 1%-2% | 2%-4% | 8%-10% | 0%-1% |
| Ex 320 | 0%-1% | 1%-2% | 2%-4% | 8%-10% | 1%-2% |
| Ex 321 | 0%-1% | 1%-2% | 2%-4% | 8%-10% | 2%-4% |
| Ex 322 | 0%-1% | 1%-2% | 2%-4% | 8%-10% | 4%-6% |
| Ex 323 | 0%-1% | 1%-2% | 2%-4% | 8%-10% | 6%-8% |
| Ex 324 | 0%-1% | 1%-2% | 2%-4% | 8%-10% | 8%-10% |
| Ex 325 | 0%-1% | 1%-2% | 4%-6% | 0%-1% | 0%-1% |
| Ex 326 | 0%-1% | 1%-2% | 4%-6% | 0%-1% | 1%-2% |
| Ex 327 | 0%-1% | 1%-2% | 4%-6% | 0%-1% | 2%-4% |
| Ex 328 | 0%-1% | 1%-2% | 4%-6% | 0%-1% | 4%-6% |
| Ex 329 | 0%-1% | 1%-2% | 4%-6% | 0%-1% | 6%-8% |
| Ex 330 | 0%-1% | 1%-2% | 4%-6% | 0%-1% | 8%-10% |
| Ex 331 | 0%-1% | 1%-2% | 4%-6% | 1%-2% | 0%-1% |
| Ex 332 | 0%-1% | 1%-2% | 4%-6% | 1%-2% | 1%-2% |
| Ex 333 | 0%-1% | 1%-2% | 4%-6% | 1%-2% | 2%-4% |
| Ex 334 | 0%-1% | 1%-2% | 4%-6% | 1%-2% | 4%-6% |
| Ex 335 | 0%-1% | 1%-2% | 4%-6% | 1%-2% | 6%-8% |
| Ex 336 | 0%-1% | 1%-2% | 4%-6% | 1%-2% | 8%-10% |
| Ex 337 | 0%-1% | 1%-2% | 4%-6% | 2%-4% | 0%-1% |
| Ex 338 | 0%-1% | 1%-2% | 4%-6% | 2%-4% | 1%-2% |
| Ex 339 | 0%-1% | 1%-2% | 4%-6% | 2%-4% | 2%-4% |
| Ex 340 | 0%-1% | 1%-2% | 4%-6% | 2%-4% | 4%-6% |
| Ex 341 | 0%-1% | 1%-2% | 4%-6% | 2%-4% | 6%-8% |
| Ex 342 | 0%-1% | 1%-2% | 4%-6% | 2%-4% | 8%-10% |
| Ex 343 | 0%-1% | 1%-2% | 4%-6% | 4%-6% | 0%-1% |
| Ex 344 | 0%-1% | 1%-2% | 4%-6% | 4%-6% | 1%-2% |
| Ex 345 | 0%-1% | 1%-2% | 4%-6% | 4%-6% | 2%-4% |
| Ex 346 | 0%-1% | 1%-2% | 4%-6% | 4%-6% | 4%-6% |
| Ex 347 | 0%-1% | 1%-2% | 4%-6% | 4%-6% | 6%-8% |
| Ex 348 | 0%-1% | 1%-2% | 4%-6% | 4%-6% | 8%-10% |
| Ex 349 | 0%-1% | 1%-2% | 4%-6% | 6%-8% | 0%-1% |
| Ex 350 | 0%-1% | 1%-2% | 4%-6% | 6%-8% | 1%-2% |
| Ex 351 | 0%-1% | 1%-2% | 4%-6% | 6%-8% | 2%-4% |
| Ex 352 | 0%-1% | 1%-2% | 4%-6% | 6%-8% | 4%-6% |
| Ex 353 | 0%-1% | 1%-2% | 4%-6% | 6%-8% | 6%-8% |
| Ex 354 | 0%-1% | 1%-2% | 4%-6% | 6%-8% | 8%-10% |
| Ex 355 | 0%-1% | 1%-2% | 4%-6% | 8%-10% | 0%-1% |
| Ex 356 | 0%-1% | 1%-2% | 4%-6% | 8%-10% | 1%-2% |
| Ex 357 | 0%-1% | 1%-2% | 4%-6% | 8%-10% | 2%-4% |
| Ex 358 | 0%-1% | 1%-2% | 4%-6% | 8%-10% | 4%-6% |
| Ex 359 | 0%-1% | 1%-2% | 4%-6% | 8%-10% | 6%-8% |
| Ex 360 | 0%-1% | 1%-2% | 4%-6% | 8%-10% | 8%-10% |
| Ex 361 | 0%-1% | 1%-2% | 6%-8% | 0%-1% | 0%-1% |
| Ex 362 | 0%-1% | 1%-2% | 6%-8% | 0%-1% | 1%-2% |
| Ex 363 | 0%-1% | 1%-2% | 6%-8% | 0%-1% | 2%-4% |
| Ex 364 | 0%-1% | 1%-2% | 6%-8% | 0%-1% | 4%-6% |
| Ex 365 | 0%-1% | 1%-2% | 6%-8% | 0%-1% | 6%-8% |
| Ex 366 | 0%-1% | 1%-2% | 6%-8% | 0%-1% | 8%-10% |
| Ex 367 | 0%-1% | 1%-2% | 6%-8% | 1%-2% | 0%-1% |
| Ex 368 | 0%-1% | 1%-2% | 6%-8% | 1%-2% | 1%-2% |
| Ex 369 | 0%-1% | 1%-2% | 6%-8% | 1%-2% | 2%-4% |
| Ex 370 | 0%-1% | 1%-2% | 6%-8% | 1%-2% | 4%-6% |
| Ex 371 | 0%-1% | 1%-2% | 6%-8% | 1%-2% | 6%-8% |
| Ex 372 | 0%-1% | 1%-2% | 6%-8% | 1%-2% | 8%-10% |
| Ex 373 | 0%-1% | 1%-2% | 6%-8% | 2%-4% | 0%-1% |
| Ex 374 | 0%-1% | 1%-2% | 6%-8% | 2%-4% | 1%-2% |
| Ex 375 | 0%-1% | 1%-2% | 6%-8% | 2%-4% | 2%-4% |
| Ex 376 | 0%-1% | 1%-2% | 6%-8% | 2%-4% | 4%-6% |
| Ex 377 | 0%-1% | 1%-2% | 6%-8% | 2%-4% | 6%-8% |
| Ex 378 | 0%-1% | 1%-2% | 6%-8% | 2%-4% | 8%-10% |
| Ex 379 | 0%-1% | 1%-2% | 6%-8% | 4%-6% | 0%-1% |
| Ex 380 | 0%-1% | 1%-2% | 6%-8% | 4%-6% | 1%-2% |
| Ex 381 | 0%-1% | 1%-2% | 6%-8% | 4%-6% | 2%-4% |
| Ex 382 | 0%-1% | 1%-2% | 6%-8% | 4%-6% | 4%-6% |
| Ex 383 | 0%-1% | 1%-2% | 6%-8% | 4%-6% | 6%-8% |
| Ex 384 | 0%-1% | 1%-2% | 6%-8% | 4%-6% | 8%-10% |
| Ex 385 | 0%-1% | 1%-2% | 6%-8% | 6%-8% | 0%-1% |
| Ex 386 | 0%-1% | 1%-2% | 6%-8% | 6%-8% | 1%-2% |
| Ex 387 | 0%-1% | 1%-2% | 6%-8% | 6%-8% | 2%-4% |
| Ex 388 | 0%-1% | 1%-2% | 6%-8% | 6%-8% | 4%-6% |
| Ex 389 | 0%-1% | 1%-2% | 6%-8% | 6%-8% | 6%-8% |
| Ex 390 | 0%-1% | 1%-2% | 6%-8% | 6%-8% | 8%-10% |
| Ex 391 | 0%-1% | 1%-2% | 6%-8% | 8%-10% | 0%-1% |
| Ex 392 | 0%-1% | 1%-2% | 6%-8% | 8%-10% | 1%-2% |
| Ex 393 | 0%-1% | 1%-2% | 6%-8% | 8%-10% | 2%-4% |
| Ex 394 | 0%-1% | 1%-2% | 6%-8% | 8%-10% | 4%-6% |
| Ex 395 | 0%-1% | 1%-2% | 6%-8% | 8%-10% | 6%-8% |
| Ex 396 | 0%-1% | 1%-2% | 6%-8% | 8%-10% | 8%-10% |
| Ex 397 | 0%-1% | 1%-2% | 8%-10% | 0%-1% | 0%-1% |
| Ex 398 | 0%-1% | 1%-2% | 8%-10% | 0%-1% | 1%-2% |
| Ex 399 | 0%-1% | 1%-2% | 8%-10% | 0%-1% | 2%-4% |
| Ex 400 | 0%-1% | 1%-2% | 8%-10% | 0%-1% | 4%-6% |
| Ex 401 | 0%-1% | 1%-2% | 8%-10% | 0%-1% | 6%-8% |
| Ex 402 | 0%-1% | 1%-2% | 8%-10% | 0%-1% | 8%-10% |
| Ex 403 | 0%-1% | 1%-2% | 8%-10% | 1%-2% | 0%-1% |
| Ex 404 | 0%-1% | 1%-2% | 8%-10% | 1%-2% | 1%-2% |
| Ex 405 | 0%-1% | 1%-2% | 8%-10% | 1%-2% | 2%-4% |
| Ex 406 | 0%-1% | 1%-2% | 8%-10% | 1%-2% | 4%-6% |
| Ex 407 | 0%-1% | 1%-2% | 8%-10% | 1%-2% | 6%-8% |
| Ex 408 | 0%-1% | 1%-2% | 8%-10% | 1%-2% | 8%-10% |
| Ex 409 | 0%-1% | 1%-2% | 8%-10% | 2%-4% | 0%-1% |
| Ex 410 | 0%-1% | 1%-2% | 8%-10% | 2%-4% | 1%-2% |
| Ex 411 | 0%-1% | 1%-2% | 8%-10% | 2%-4% | 2%-4% |
| Ex 412 | 0%-1% | 1%-2% | 8%-10% | 2%-4% | 4%-6% |
| Ex 413 | 0%-1% | 1%-2% | 8%-10% | 2%-4% | 6%-8% |
| Ex 414 | 0%-1% | 1%-2% | 8%-10% | 2%-4% | 8%-10% |
| Ex 415 | 0%-1% | 1%-2% | 8%-10% | 4%-6% | 0%-1% |
| Ex 416 | 0%-1% | 1%-2% | 8%-10% | 4%-6% | 1%-2% |
| Ex 417 | 0%-1% | 1%-2% | 8%-10% | 4%-6% | 2%-4% |
| Ex 418 | 0%-1% | 1%-2% | 8%-10% | 4%-6% | 4%-6% |
| Ex 419 | 0%-1% | 1%-2% | 8%-10% | 4%-6% | 6%-8% |
| Ex 420 | 0%-1% | 1%-2% | 8%-10% | 4%-6% | 8%-10% |
| Ex 421 | 0%-1% | 1%-2% | 8%-10% | 6%-8% | 0%-1% |
| Ex 422 | 0%-1% | 1%-2% | 8%-10% | 6%-8% | 1%-2% |
| Ex 423 | 0%-1% | 1%-2% | 8%-10% | 6%-8% | 2%-4% |
| Ex 424 | 0%-1% | 1%-2% | 8%-10% | 6%-8% | 4%-6% |
| Ex 425 | 0%-1% | 1%-2% | 8%-10% | 6%-8% | 6%-8% |
| Ex 426 | 0%-1% | 1%-2% | 8%-10% | 6%-8% | 8%-10% |
| Ex 427 | 0%-1% | 1%-2% | 8%-10% | 8%-10% | 0%-1% |
| Ex 428 | 0%-1% | 1%-2% | 8%-10% | 8%-10% | 1%-2% |
| Ex 429 | 0%-1% | 1%-2% | 8%-10% | 8%-10% | 2%-4% |
| Ex 430 | 0%-1% | 1%-2% | 8%-10% | 8%-10% | 4%-6% |
| Ex 431 | 0%-1% | 1%-2% | 8%-10% | 8%-10% | 6%-8% |
| Ex 432 | 0%-1% | 1%-2% | 8%-10% | 8%-10% | 8%-10% |
| Ex 433 | 0%-1% | 2%-4% | 0%-1% | 0%-1% | 0%-1% |
| Ex 434 | 0%-1% | 2%-4% | 0%-1% | 0%-1% | 1%-2% |
| Ex 435 | 0%-1% | 2%-4% | 0%-1% | 0%-1% | 2%-4% |
| Ex 436 | 0%-1% | 2%-4% | 0%-1% | 0%-1% | 4%-6% |
| Ex 437 | 0%-1% | 2%-4% | 0%-1% | 0%-1% | 6%-8% |
| Ex 438 | 0%-1% | 2%-4% | 0%-1% | 0%-1% | 8%-10% |
| Ex 439 | 0%-1% | 2%-4% | 0%-1% | 1%-2% | 0%-1% |
| Ex 440 | 0%-1% | 2%-4% | 0%-1% | 1%-2% | 1%-2% |
| Ex 441 | 0%-1% | 2%-4% | 0%-1% | 1%-2% | 2%-4% |
| Ex 442 | 0%-1% | 2%-4% | 0%-1% | 1%-2% | 4%-6% |
| Ex 443 | 0%-1% | 2%-4% | 0%-1% | 1%-2% | 6%-8% |
| Ex 444 | 0%-1% | 2%-4% | 0%-1% | 1%-2% | 8%-10% |
| Ex 445 | 0%-1% | 2%-4% | 0%-1% | 2%-4% | 0%-1% |
| Ex 446 | 0%-1% | 2%-4% | 0%-1% | 2%-4% | 1%-2% |
| Ex 447 | 0%-1% | 2%-4% | 0%-1% | 2%-4% | 2%-4% |
| Ex 448 | 0%-1% | 2%-4% | 0%-1% | 2%-4% | 4%-6% |
| Ex 449 | 0%-1% | 2%-4% | 0%-1% | 2%-4% | 6%-8% |
| Ex 450 | 0%-1% | 2%-4% | 0%-1% | 2%-4% | 8%-10% |
| Ex 451 | 0%-1% | 2%-4% | 0%-1% | 4%-6% | 0%-1% |
| Ex 452 | 0%-1% | 2%-4% | 0%-1% | 4%-6% | 1%-2% |
| Ex 453 | 0%-1% | 2%-4% | 0%-1% | 4%-6% | 2%-4% |
| Ex 454 | 0%-1% | 2%-4% | 0%-1% | 4%-6% | 4%-6% |
| Ex 455 | 0%-1% | 2%-4% | 0%-1% | 4%-6% | 6%-8% |
| Ex 456 | 0%-1% | 2%-4% | 0%-1% | 4%-6% | 8%-10% |
| Ex 457 | 0%-1% | 2%-4% | 0%-1% | 6%-8% | 0%-1% |
| Ex 458 | 0%-1% | 2%-4% | 0%-1% | 6%-8% | 1%-2% |
| Ex 459 | 0%-1% | 2%-4% | 0%-1% | 6%-8% | 2%-4% |
| Ex 460 | 0%-1% | 2%-4% | 0%-1% | 6%-8% | 4%-6% |
| Ex 461 | 0%-1% | 2%-4% | 0%-1% | 6%-8% | 6%-8% |
| Ex 462 | 0%-1% | 2%-4% | 0%-1% | 6%-8% | 8%-10% |
| Ex 463 | 0%-1% | 2%-4% | 0%-1% | 8%-10% | 0%-1% |
| Ex 464 | 0%-1% | 2%-4% | 0%-1% | 8%-10% | 1%-2% |
| Ex 465 | 0%-1% | 2%-4% | 0%-1% | 8%-10% | 2%-4% |
| Ex 466 | 0%-1% | 2%-4% | 0%-1% | 8%-10% | 4%-6% |
| Ex 467 | 0%-1% | 2%-4% | 0%-1% | 8%-10% | 6%-8% |
| Ex 468 | 0%-1% | 2%-4% | 0%-1% | 8%-10% | 8%-10% |
| Ex 469 | 0%-1% | 2%-4% | 1%-2% | 0%-1% | 0%-1% |
| Ex 470 | 0%-1% | 2%-4% | 1%-2% | 0%-1% | 1%-2% |
| Ex 471 | 0%-1% | 2%-4% | 1%-2% | 0%-1% | 2%-4% |
| Ex 472 | 0%-1% | 2%-4% | 1%-2% | 0%-1% | 4%-6% |
| Ex 473 | 0%-1% | 2%-4% | 1%-2% | 0%-1% | 6%-8% |
| Ex 474 | 0%-1% | 2%-4% | 1%-2% | 0%-1% | 8%-10% |
| Ex 475 | 0%-1% | 2%-4% | 1%-2% | 1%-2% | 0%-1% |
| Ex 476 | 0%-1% | 2%-4% | 1%-2% | 1%-2% | 1%-2% |
| Ex 477 | 0%-1% | 2%-4% | 1%-2% | 1%-2% | 2%-4% |
| Ex 478 | 0%-1% | 2%-4% | 1%-2% | 1%-2% | 4%-6% |
| Ex 479 | 0%-1% | 2%-4% | 1%-2% | 1%-2% | 6%-8% |
| Ex 480 | 0%-1% | 2%-4% | 1%-2% | 1%-2% | 8%-10% |
| Ex 481 | 0%-1% | 2%-4% | 1%-2% | 2%-4% | 0%-1% |
| Ex 482 | 0%-1% | 2%-4% | 1%-2% | 2%-4% | 1%-2% |
| Ex 483 | 0%-1% | 2%-4% | 1%-2% | 2%-4% | 2%-4% |
| Ex 484 | 0%-1% | 2%-4% | 1%-2% | 2%-4% | 4%-6% |
| Ex 485 | 0%-1% | 2%-4% | 1%-2% | 2%-4% | 6%-8% |
| Ex 486 | 0%-1% | 2%-4% | 1%-2% | 2%-4% | 8%-10% |
| Ex 487 | 0%-1% | 2%-4% | 1%-2% | 4%-6% | 0%-1% |
| Ex 488 | 0%-1% | 2%-4% | 1%-2% | 4%-6% | 1%-2% |
| Ex 489 | 0%-1% | 2%-4% | 1%-2% | 4%-6% | 2%-4% |
| Ex 490 | 0%-1% | 2%-4% | 1%-2% | 4%-6% | 4%-6% |
| Ex 491 | 0%-1% | 2%-4% | 1%-2% | 4%-6% | 6%-8% |
| Ex 492 | 0%-1% | 2%-4% | 1%-2% | 4%-6% | 8%-10% |
| Ex 493 | 0%-1% | 2%-4% | 1%-2% | 6%-8% | 0%-1% |
| Ex 494 | 0%-1% | 2%-4% | 1%-2% | 6%-8% | 1%-2% |
| Ex 495 | 0%-1% | 2%-4% | 1%-2% | 6%-8% | 2%-4% |
| Ex 496 | 0%-1% | 2%-4% | 1%-2% | 6%-8% | 4%-6% |
| Ex 497 | 0%-1% | 2%-4% | 1%-2% | 6%-8% | 6%-8% |
| Ex 498 | 0%-1% | 2%-4% | 1%-2% | 6%-8% | 8%-10% |
| Ex 499 | 0%-1% | 2%-4% | 1%-2% | 8%-10% | 0%-1% |
| Ex 500 | 0%-1% | 2%-4% | 1%-2% | 8%-10% | 1%-2% |
| Ex 501 | 0%-1% | 2%-4% | 1%-2% | 8%-10% | 2%-4% |
| Ex 502 | 0%-1% | 2%-4% | 1%-2% | 8%-10% | 4%-6% |
| Ex 503 | 0%-1% | 2%-4% | 1%-2% | 8%-10% | 6%-8% |
| Ex 504 | 0%-1% | 2%-4% | 1%-2% | 8%-10% | 8%-10% |
| Ex 505 | 0%-1% | 2%-4% | 2%-4% | 0%-1% | 0%-1% |
| Ex 506 | 0%-1% | 2%-4% | 2%-4% | 0%-1% | 1%-2% |
| Ex 507 | 0%-1% | 2%-4% | 2%-4% | 0%-1% | 2%-4% |
| Ex 508 | 0%-1% | 2%-4% | 2%-4% | 0%-1% | 4%-6% |
| Ex 509 | 0%-1% | 2%-4% | 2%-4% | 0%-1% | 6%-8% |
| Ex 510 | 0%-1% | 2%-4% | 2%-4% | 0%-1% | 8%-10% |
| Ex 511 | 0%-1% | 2%-4% | 2%-4% | 1%-2% | 0%-1% |
| Ex 512 | 0%-1% | 2%-4% | 2%-4% | 1%-2% | 1%-2% |
| Ex 513 | 0%-1% | 2%-4% | 2%-4% | 1%-2% | 2%-4% |
| Ex 514 | 0%-1% | 2%-4% | 2%-4% | 1%-2% | 4%-6% |
| Ex 515 | 0%-1% | 2%-4% | 2%-4% | 1%-2% | 6%-8% |
| Ex 516 | 0%-1% | 2%-4% | 2%-4% | 1%-2% | 8%-10% |
| Ex 517 | 0%-1% | 2%-4% | 2%-4% | 2%-4% | 0%-1% |
| Ex 518 | 0%-1% | 2%-4% | 2%-4% | 2%-4% | 1%-2% |
| Ex 519 | 0%-1% | 2%-4% | 2%-4% | 2%-4% | 2%-4% |
| Ex 520 | 0%-1% | 2%-4% | 2%-4% | 2%-4% | 4%-6% |
| Ex 521 | 0%-1% | 2%-4% | 2%-4% | 2%-4% | 6%-8% |
| Ex 522 | 0%-1% | 2%-4% | 2%-4% | 2%-4% | 8%-10% |
| Ex 523 | 0%-1% | 2%-4% | 2%-4% | 4%-6% | 0%-1% |
| Ex 524 | 0%-1% | 2%-4% | 2%-4% | 4%-6% | 1%-2% |
| Ex 525 | 0%-1% | 2%-4% | 2%-4% | 4%-6% | 2%-4% |
| Ex 526 | 0%-1% | 2%-4% | 2%-4% | 4%-6% | 4%-6% |
| Ex 527 | 0%-1% | 2%-4% | 2%-4% | 4%-6% | 6%-8% |
| Ex 528 | 0%-1% | 2%-4% | 2%-4% | 4%-6% | 8%-10% |
| Ex 529 | 0%-1% | 2%-4% | 2%-4% | 6%-8% | 0%-1% |
| Ex 530 | 0%-1% | 2%-4% | 2%-4% | 6%-8% | 1%-2% |
| Ex 531 | 0%-1% | 2%-4% | 2%-4% | 6%-8% | 2%-4% |
| Ex 532 | 0%-1% | 2%-4% | 2%-4% | 6%-8% | 4%-6% |
| Ex 533 | 0%-1% | 2%-4% | 2%-4% | 6%-8% | 6%-8% |
| Ex 534 | 0%-1% | 2%-4% | 2%-4% | 6%-8% | 8%-10% |
| Ex 535 | 0%-1% | 2%-4% | 2%-4% | 8%-10% | 0%-1% |
| Ex 536 | 0%-1% | 2%-4% | 2%-4% | 8%-10% | 1%-2% |
| Ex 537 | 0%-1% | 2%-4% | 2%-4% | 8%-10% | 2%-4% |
| Ex 538 | 0%-1% | 2%-4% | 2%-4% | 8%-10% | 4%-6% |
| Ex 539 | 0%-1% | 2%-4% | 2%-4% | 8%-10% | 6%-8% |
| Ex 540 | 0%-1% | 2%-4% | 2%-4% | 8%-10% | 8%-10% |
| Ex 541 | 0%-1% | 2%-4% | 4%-6% | 0%-1% | 0%-1% |
| Ex 542 | 0%-1% | 2%-4% | 4%-6% | 0%-1% | 1%-2% |
| Ex 543 | 0%-1% | 2%-4% | 4%-6% | 0%-1% | 2%-4% |
| Ex 544 | 0%-1% | 2%-4% | 4%-6% | 0%-1% | 4%-6% |
| Ex 545 | 0%-1% | 2%-4% | 4%-6% | 0%-1% | 6%-8% |
| Ex 546 | 0%-1% | 2%-4% | 4%-6% | 0%-1% | 8%-10% |
| Ex 547 | 0%-1% | 2%-4% | 4%-6% | 1%-2% | 0%-1% |
| Ex 548 | 0%-1% | 2%-4% | 4%-6% | 1%-2% | 1%-2% |
| Ex 549 | 0%-1% | 2%-4% | 4%-6% | 1%-2% | 2%-4% |
| Ex 550 | 0%-1% | 2%-4% | 4%-6% | 1%-2% | 4%-6% |
| Ex 551 | 0%-1% | 2%-4% | 4%-6% | 1%-2% | 6%-8% |
| Ex 552 | 0%-1% | 2%-4% | 4%-6% | 1%-2% | 8%-10% |
| Ex 553 | 0%-1% | 2%-4% | 4%-6% | 2%-4% | 0%-1% |
| Ex 554 | 0%-1% | 2%-4% | 4%-6% | 2%-4% | 1%-2% |
| Ex 555 | 0%-1% | 2%-4% | 4%-6% | 2%-4% | 2%-4% |
| Ex 556 | 0%-1% | 2%-4% | 4%-6% | 2%-4% | 4%-6% |
| Ex 557 | 0%-1% | 2%-4% | 4%-6% | 2%-4% | 6%-8% |
| Ex 558 | 0%-1% | 2%-4% | 4%-6% | 2%-4% | 8%-10% |
| Ex 559 | 0%-1% | 2%-4% | 4%-6% | 4%-6% | 0%-1% |
| Ex 560 | 0%-1% | 2%-4% | 4%-6% | 4%-6% | 1%-2% |
| Ex 561 | 0%-1% | 2%-4% | 4%-6% | 4%-6% | 2%-4% |
| Ex 562 | 0%-1% | 2%-4% | 4%-6% | 4%-6% | 4%-6% |
| Ex 563 | 0%-1% | 2%-4% | 4%-6% | 4%-6% | 6%-8% |
| Ex 564 | 0%-1% | 2%-4% | 4%-6% | 4%-6% | 8%-10% |
| Ex 565 | 0%-1% | 2%-4% | 4%-6% | 6%-8% | 0%-1% |
| Ex 566 | 0%-1% | 2%-4% | 4%-6% | 6%-8% | 1%-2% |
| Ex 567 | 0%-1% | 2%-4% | 4%-6% | 6%-8% | 2%-4% |
| Ex 568 | 0%-1% | 2%-4% | 4%-6% | 6%-8% | 4%-6% |
| Ex 569 | 0%-1% | 2%-4% | 4%-6% | 6%-8% | 6%-8% |
| Ex 570 | 0%-1% | 2%-4% | 4%-6% | 6%-8% | 8%-10% |
| Ex 571 | 0%-1% | 2%-4% | 4%-6% | 8%-10% | 0%-1% |
| Ex 572 | 0%-1% | 2%-4% | 4%-6% | 8%-10% | 1%-2% |
| Ex 573 | 0%-1% | 2%-4% | 4%-6% | 8%-10% | 2%-4% |
| Ex 574 | 0%-1% | 2%-4% | 4%-6% | 8%-10% | 4%-6% |
| Ex 575 | 0%-1% | 2%-4% | 4%-6% | 8%-10% | 6%-8% |
| Ex 576 | 0%-1% | 2%-4% | 4%-6% | 8%-10% | 8%-10% |
| Ex 577 | 0%-1% | 2%-4% | 6%-8% | 0%-1% | 0%-1% |
| Ex 578 | 0%-1% | 2%-4% | 6%-8% | 0%-1% | 1%-2% |
| Ex 579 | 0%-1% | 2%-4% | 6%-8% | 0%-1% | 2%-4% |
| Ex 580 | 0%-1% | 2%-4% | 6%-8% | 0%-1% | 4%-6% |
| Ex 581 | 0%-1% | 2%-4% | 6%-8% | 0%-1% | 6%-8% |
| Ex 582 | 0%-1% | 2%-4% | 6%-8% | 0%-1% | 8%-10% |
| Ex 583 | 0%-1% | 2%-4% | 6%-8% | 1%-2% | 0%-1% |
| Ex 584 | 0%-1% | 2%-4% | 6%-8% | 1%-2% | 1%-2% |
| Ex 585 | 0%-1% | 2%-4% | 6%-8% | 1%-2% | 2%-4% |
| Ex 586 | 0%-1% | 2%-4% | 6%-8% | 1%-2% | 4%-6% |
| Ex 587 | 0%-1% | 2%-4% | 6%-8% | 1%-2% | 6%-8% |
| Ex 588 | 0%-1% | 2%-4% | 6%-8% | 1%-2% | 8%-10% |
| Ex 589 | 0%-1% | 2%-4% | 6%-8% | 2%-4% | 0%-1% |
| Ex 590 | 0%-1% | 2%-4% | 6%-8% | 2%-4% | 1%-2% |
| Ex 591 | 0%-1% | 2%-4% | 6%-8% | 2%-4% | 2%-4% |
| Ex 592 | 0%-1% | 2%-4% | 6%-8% | 2%-4% | 4%-6% |
| Ex 593 | 0%-1% | 2%-4% | 6%-8% | 2%-4% | 6%-8% |
| Ex 594 | 0%-1% | 2%-4% | 6%-8% | 2%-4% | 8%-10% |
| Ex 595 | 0%-1% | 2%-4% | 6%-8% | 4%-6% | 0%-1% |
| Ex 596 | 0%-1% | 2%-4% | 6%-8% | 4%-6% | 1%-2% |
| Ex 597 | 0%-1% | 2%-4% | 6%-8% | 4%-6% | 2%-4% |
| Ex 598 | 0%-1% | 2%-4% | 6%-8% | 4%-6% | 4%-6% |
| Ex 599 | 0%-1% | 2%-4% | 6%-8% | 4%-6% | 6%-8% |
| Ex 600 | 0%-1% | 2%-4% | 6%-8% | 4%-6% | 8%-10% |
| Ex 601 | 0%-1% | 2%-4% | 6%-8% | 6%-8% | 0%-1% |
| Ex 602 | 0%-1% | 2%-4% | 6%-8% | 6%-8% | 1%-2% |
| Ex 603 | 0%-1% | 2%-4% | 6%-8% | 6%-8% | 2%-4% |
| Ex 604 | 0%-1% | 2%-4% | 6%-8% | 6%-8% | 4%-6% |
| Ex 605 | 0%-1% | 2%-4% | 6%-8% | 6%-8% | 6%-8% |
| Ex 606 | 0%-1% | 2%-4% | 6%-8% | 6%-8% | 8%-10% |
| Ex 607 | 0%-1% | 2%-4% | 6%-8% | 8%-10% | 0%-1% |
| Ex 608 | 0%-1% | 2%-4% | 6%-8% | 8%-10% | 1%-2% |
| Ex 609 | 0%-1% | 2%-4% | 6%-8% | 8%-10% | 2%-4% |
| Ex 610 | 0%-1% | 2%-4% | 6%-8% | 8%-10% | 4%-6% |
| Ex 611 | 0%-1% | 2%-4% | 6%-8% | 8%-10% | 6%-8% |
| Ex 612 | 0%-1% | 2%-4% | 6%-8% | 8%-10% | 8%-10% |
| Ex 613 | 0%-1% | 2%-4% | 8%-10% | 0%-1% | 0%-1% |
| Ex 614 | 0%-1% | 2%-4% | 8%-10% | 0%-1% | 1%-2% |
| Ex 615 | 0%-1% | 2%-4% | 8%-10% | 0%-1% | 2%-4% |
| Ex 616 | 0%-1% | 2%-4% | 8%-10% | 0%-1% | 4%-6% |
| Ex 617 | 0%-1% | 2%-4% | 8%-10% | 0%-1% | 6%-8% |
| Ex 618 | 0%-1% | 2%-4% | 8%-10% | 0%-1% | 8%-10% |
| Ex 619 | 0%-1% | 2%-4% | 8%-10% | 1%-2% | 0%-1% |
| Ex 620 | 0%-1% | 2%-4% | 8%-10% | 1%-2% | 1%-2% |
| Ex 621 | 0%-1% | 2%-4% | 8%-10% | 1%-2% | 2%-4% |
| Ex 622 | 0%-1% | 2%-4% | 8%-10% | 1%-2% | 4%-6% |
| Ex 623 | 0%-1% | 2%-4% | 8%-10% | 1%-2% | 6%-8% |
| Ex 624 | 0%-1% | 2%-4% | 8%-10% | 1%-2% | 8%-10% |
| Ex 625 | 0%-1% | 2%-4% | 8%-10% | 2%-4% | 0%-1% |
| Ex 626 | 0%-1% | 2%-4% | 8%-10% | 2%-4% | 1%-2% |
| Ex 627 | 0%-1% | 2%-4% | 8%-10% | 2%-4% | 2%-4% |
| Ex 628 | 0%-1% | 2%-4% | 8%-10% | 2%-4% | 4%-6% |
| Ex 629 | 0%-1% | 2%-4% | 8%-10% | 2%-4% | 6%-8% |
| Ex 630 | 0%-1% | 2%-4% | 8%-10% | 2%-4% | 8%-10% |
| Ex 631 | 0%-1% | 2%-4% | 8%-10% | 4%-6% | 0%-1% |
| Ex 632 | 0%-1% | 2%-4% | 8%-10% | 4%-6% | 1%-2% |
| Ex 633 | 0%-1% | 2%-4% | 8%-10% | 4%-6% | 2%-4% |
| Ex 634 | 0%-1% | 2%-4% | 8%-10% | 4%-6% | 4%-6% |
| Ex 635 | 0%-1% | 2%-4% | 8%-10% | 4%-6% | 6%-8% |
| Ex 636 | 0%-1% | 2%-4% | 8%-10% | 4%-6% | 8%-10% |
| Ex 637 | 0%-1% | 2%-4% | 8%-10% | 6%-8% | 0%-1% |
| Ex 638 | 0%-1% | 2%-4% | 8%-10% | 6%-8% | 1%-2% |
| Ex 639 | 0%-1% | 2%-4% | 8%-10% | 6%-8% | 2%-4% |
| Ex 640 | 0%-1% | 2%-4% | 8%-10% | 6%-8% | 4%-6% |
| Ex 641 | 0%-1% | 2%-4% | 8%-10% | 6%-8% | 6%-8% |
| Ex 642 | 0%-1% | 2%-4% | 8%-10% | 6%-8% | 8%-10% |
| Ex 643 | 0%-1% | 2%-4% | 8%-10% | 8%-10% | 0%-1% |
| Ex 644 | 0%-1% | 2%-4% | 8%-10% | 8%-10% | 1%-2% |
| Ex 645 | 0%-1% | 2%-4% | 8%-10% | 8%-10% | 2%-4% |
| Ex 646 | 0%-1% | 2%-4% | 8%-10% | 8%-10% | 4%-6% |
| Ex 647 | 0%-1% | 2%-4% | 8%-10% | 8%-10% | 6%-8% |
| Ex 648 | 0%-1% | 2%-4% | 8%-10% | 8%-10% | 8%-10% |
| Ex 649 | 0%-1% | 4%-6% | 0%-1% | 0%-1% | 0%-1% |
| Ex 650 | 0%-1% | 4%-6% | 0%-1% | 0%-1% | 1%-2% |
| Ex 651 | 0%-1% | 4%-6% | 0%-1% | 0%-1% | 2%-4% |
| Ex 652 | 0%-1% | 4%-6% | 0%-1% | 0%-1% | 4%-6% |
| Ex 653 | 0%-1% | 4%-6% | 0%-1% | 0%-1% | 6%-8% |
| Ex 654 | 0%-1% | 4%-6% | 0%-1% | 0%-1% | 8%-10% |
| Ex 655 | 0%-1% | 4%-6% | 0%-1% | 1%-2% | 0%-1% |
| Ex 656 | 0%-1% | 4%-6% | 0%-1% | 1%-2% | 1%-2% |
| Ex 657 | 0%-1% | 4%-6% | 0%-1% | 1%-2% | 2%-4% |
| Ex 658 | 0%-1% | 4%-6% | 0%-1% | 1%-2% | 4%-6% |
| Ex 659 | 0%-1% | 4%-6% | 0%-1% | 1%-2% | 6%-8% |
| Ex 660 | 0%-1% | 4%-6% | 0%-1% | 1%-2% | 8%-10% |
| Ex 661 | 0%-1% | 4%-6% | 0%-1% | 2%-4% | 0%-1% |
| Ex 662 | 0%-1% | 4%-6% | 0%-1% | 2%-4% | 1%-2% |
| Ex 663 | 0%-1% | 4%-6% | 0%-1% | 2%-4% | 2%-4% |
| Ex 664 | 0%-1% | 4%-6% | 0%-1% | 2%-4% | 4%-6% |
| Ex 665 | 0%-1% | 4%-6% | 0%-1% | 2%-4% | 6%-8% |
| Ex 666 | 0%-1% | 4%-6% | 0%-1% | 2%-4% | 8%-10% |
| Ex 667 | 0%-1% | 4%-6% | 0%-1% | 4%-6% | 0%-1% |
| Ex 668 | 0%-1% | 4%-6% | 0%-1% | 4%-6% | 1%-2% |
| Ex 669 | 0%-1% | 4%-6% | 0%-1% | 4%-6% | 2%-4% |
| Ex 670 | 0%-1% | 4%-6% | 0%-1% | 4%-6% | 4%-6% |
| Ex 671 | 0%-1% | 4%-6% | 0%-1% | 4%-6% | 6%-8% |
| Ex 672 | 0%-1% | 4%-6% | 0%-1% | 4%-6% | 8%-10% |
| Ex 673 | 0%-1% | 4%-6% | 0%-1% | 6%-8% | 0%-1% |
| Ex 674 | 0%-1% | 4%-6% | 0%-1% | 6%-8% | 1%-2% |
| Ex 675 | 0%-1% | 4%-6% | 0%-1% | 6%-8% | 2%-4% |
| Ex 676 | 0%-1% | 4%-6% | 0%-1% | 6%-8% | 4%-6% |
| Ex 677 | 0%-1% | 4%-6% | 0%-1% | 6%-8% | 6%-8% |
| Ex 678 | 0%-1% | 4%-6% | 0%-1% | 6%-8% | 8%-10% |
| Ex 679 | 0%-1% | 4%-6% | 0%-1% | 8%-10% | 0%-1% |
| Ex 680 | 0%-1% | 4%-6% | 0%-1% | 8%-10% | 1%-2% |
| Ex 681 | 0%-1% | 4%-6% | 0%-1% | 8%-10% | 2%-4% |
| Ex 682 | 0%-1% | 4%-6% | 0%-1% | 8%-10% | 4%-6% |
| Ex 683 | 0%-1% | 4%-6% | 0%-1% | 8%-10% | 6%-8% |
| Ex 684 | 0%-1% | 4%-6% | 0%-1% | 8%-10% | 8%-10% |
| Ex 685 | 0%-1% | 4%-6% | 1%-2% | 0%-1% | 0%-1% |
| Ex 686 | 0%-1% | 4%-6% | 1%-2% | 0%-1% | 1%-2% |
| Ex 687 | 0%-1% | 4%-6% | 1%-2% | 0%-1% | 2%-4% |
| Ex 688 | 0%-1% | 4%-6% | 1%-2% | 0%-1% | 4%-6% |
| Ex 689 | 0%-1% | 4%-6% | 1%-2% | 0%-1% | 6%-8% |
| Ex 690 | 0%-1% | 4%-6% | 1%-2% | 0%-1% | 8%-10% |
| Ex 691 | 0%-1% | 4%-6% | 1%-2% | 1%-2% | 0%-1% |
| Ex 692 | 0%-1% | 4%-6% | 1%-2% | 1%-2% | 1%-2% |
| Ex 693 | 0%-1% | 4%-6% | 1%-2% | 1%-2% | 2%-4% |
| Ex 694 | 0%-1% | 4%-6% | 1%-2% | 1%-2% | 4%-6% |
| Ex 695 | 0%-1% | 4%-6% | 1%-2% | 1%-2% | 6%-8% |
| Ex 696 | 0%-1% | 4%-6% | 1%-2% | 1%-2% | 8%-10% |
| Ex 697 | 0%-1% | 4%-6% | 1%-2% | 2%-4% | 0%-1% |
| Ex 698 | 0%-1% | 4%-6% | 1%-2% | 2%-4% | 1%-2% |
| Ex 699 | 0%-1% | 4%-6% | 1%-2% | 2%-4% | 2%-4% |
| Ex 700 | 0%-1% | 4%-6% | 1%-2% | 2%-4% | 4%-6% |
| Ex 701 | 0%-1% | 4%-6% | 1%-2% | 2%-4% | 6%-8% |
| Ex 702 | 0%-1% | 4%-6% | 1%-2% | 2%-4% | 8%-10% |
| Ex 703 | 0%-1% | 4%-6% | 1%-2% | 4%-6% | 0%-1% |
| Ex 704 | 0%-1% | 4%-6% | 1%-2% | 4%-6% | 1%-2% |
| Ex 705 | 0%-1% | 4%-6% | 1%-2% | 4%-6% | 2%-4% |
| Ex 706 | 0%-1% | 4%-6% | 1%-2% | 4%-6% | 4%-6% |
| Ex 707 | 0%-1% | 4%-6% | 1%-2% | 4%-6% | 6%-8% |
| Ex 708 | 0%-1% | 4%-6% | 1%-2% | 4%-6% | 8%-10% |
| Ex 709 | 0%-1% | 4%-6% | 1%-2% | 6%-8% | 0%-1% |
| Ex 710 | 0%-1% | 4%-6% | 1%-2% | 6%-8% | 1%-2% |
| Ex 711 | 0%-1% | 4%-6% | 1%-2% | 6%-8% | 2%-4% |
| Ex 712 | 0%-1% | 4%-6% | 1%-2% | 6%-8% | 4%-6% |
| Ex 713 | 0%-1% | 4%-6% | 1%-2% | 6%-8% | 6%-8% |
| Ex 714 | 0%-1% | 4%-6% | 1%-2% | 6%-8% | 8%-10% |
| Ex 715 | 0%-1% | 4%-6% | 1%-2% | 8%-10% | 0%-1% |
| Ex 716 | 0%-1% | 4%-6% | 1%-2% | 8%-10% | 1%-2% |
| Ex 717 | 0%-1% | 4%-6% | 1%-2% | 8%-10% | 2%-4% |
| Ex 718 | 0%-1% | 4%-6% | 1%-2% | 8%-10% | 4%-6% |
| Ex 719 | 0%-1% | 4%-6% | 1%-2% | 8%-10% | 6%-8% |
| Ex 720 | 0%-1% | 4%-6% | 1%-2% | 8%-10% | 8%-10% |
| Ex 721 | 0%-1% | 4%-6% | 2%-4% | 0%-1% | 0%-1% |
| Ex 722 | 0%-1% | 4%-6% | 2%-4% | 0%-1% | 1%-2% |
| Ex 723 | 0%-1% | 4%-6% | 2%-4% | 0%-1% | 2%-4% |
| Ex 724 | 0%-1% | 4%-6% | 2%-4% | 0%-1% | 4%-6% |
| Ex 725 | 0%-1% | 4%-6% | 2%-4% | 0%-1% | 6%-8% |
| Ex 726 | 0%-1% | 4%-6% | 2%-4% | 0%-1% | 8%-10% |
| Ex 727 | 0%-1% | 4%-6% | 2%-4% | 1%-2% | 0%-1% |
| Ex 728 | 0%-1% | 4%-6% | 2%-4% | 1%-2% | 1%-2% |
| Ex 729 | 0%-1% | 4%-6% | 2%-4% | 1%-2% | 2%-4% |
| Ex 730 | 0%-1% | 4%-6% | 2%-4% | 1%-2% | 4%-6% |
| Ex 731 | 0%-1% | 4%-6% | 2%-4% | 1%-2% | 6%-8% |
| Ex 732 | 0%-1% | 4%-6% | 2%-4% | 1%-2% | 8%-10% |
| Ex 733 | 0%-1% | 4%-6% | 2%-4% | 2%-4% | 0%-1% |
| Ex 734 | 0%-1% | 4%-6% | 2%-4% | 2%-4% | 1%-2% |
| Ex 735 | 0%-1% | 4%-6% | 2%-4% | 2%-4% | 2%-4% |
| Ex 736 | 0%-1% | 4%-6% | 2%-4% | 2%-4% | 4%-6% |
| Ex 737 | 0%-1% | 4%-6% | 2%-4% | 2%-4% | 6%-8% |
| Ex 738 | 0%-1% | 4%-6% | 2%-4% | 2%-4% | 8%-10% |
| Ex 739 | 0%-1% | 4%-6% | 2%-4% | 4%-6% | 0%-1% |
| Ex 740 | 0%-1% | 4%-6% | 2%-4% | 4%-6% | 1%-2% |
| Ex 741 | 0%-1% | 4%-6% | 2%-4% | 4%-6% | 2%-4% |
| Ex 742 | 0%-1% | 4%-6% | 2%-4% | 4%-6% | 4%-6% |
| Ex 743 | 0%-1% | 4%-6% | 2%-4% | 4%-6% | 6%-8% |
| Ex 744 | 0%-1% | 4%-6% | 2%-4% | 4%-6% | 8%-10% |
| Ex 745 | 0%-1% | 4%-6% | 2%-4% | 6%-8% | 0%-1% |
| Ex 746 | 0%-1% | 4%-6% | 2%-4% | 6%-8% | 1%-2% |
| Ex 747 | 0%-1% | 4%-6% | 2%-4% | 6%-8% | 2%-4% |
| Ex 748 | 0%-1% | 4%-6% | 2%-4% | 6%-8% | 4%-6% |
| Ex 749 | 0%-1% | 4%-6% | 2%-4% | 6%-8% | 6%-8% |
| Ex 750 | 0%-1% | 4%-6% | 2%-4% | 6%-8% | 8%-10% |
| Ex 751 | 0%-1% | 4%-6% | 2%-4% | 8%-10% | 0%-1% |
| Ex 752 | 0%-1% | 4%-6% | 2%-4% | 8%-10% | 1%-2% |
| Ex 753 | 0%-1% | 4%-6% | 2%-4% | 8%-10% | 2%-4% |
| Ex 754 | 0%-1% | 4%-6% | 2%-4% | 8%-10% | 4%-6% |
| Ex 755 | 0%-1% | 4%-6% | 2%-4% | 8%-10% | 6%-8% |
| Ex 756 | 0%-1% | 4%-6% | 2%-4% | 8%-10% | 8%-10% |
| Ex 757 | 0%-1% | 4%-6% | 4%-6% | 0%-1% | 0%-1% |
| Ex 758 | 0%-1% | 4%-6% | 4%-6% | 0%-1% | 1%-2% |
| Ex 759 | 0%-1% | 4%-6% | 4%-6% | 0%-1% | 2%-4% |
| Ex 760 | 0%-1% | 4%-6% | 4%-6% | 0%-1% | 4%-6% |
| Ex 761 | 0%-1% | 4%-6% | 4%-6% | 0%-1% | 6%-8% |
| Ex 762 | 0%-1% | 4%-6% | 4%-6% | 0%-1% | 8%-10% |
| Ex 763 | 0%-1% | 4%-6% | 4%-6% | 1%-2% | 0%-1% |
| Ex 764 | 0%-1% | 4%-6% | 4%-6% | 1%-2% | 1%-2% |
| Ex 765 | 0%-1% | 4%-6% | 4%-6% | 1%-2% | 2%-4% |
| Ex 766 | 0%-1% | 4%-6% | 4%-6% | 1%-2% | 4%-6% |
| Ex 767 | 0%-1% | 4%-6% | 4%-6% | 1%-2% | 6%-8% |
| Ex 768 | 0%-1% | 4%-6% | 4%-6% | 1%-2% | 8%-10% |
| Ex 769 | 0%-1% | 4%-6% | 4%-6% | 2%-4% | 0%-1% |
| Ex 770 | 0%-1% | 4%-6% | 4%-6% | 2%-4% | 1%-2% |
| Ex 771 | 0%-1% | 4%-6% | 4%-6% | 2%-4% | 2%-4% |
| Ex 772 | 0%-1% | 4%-6% | 4%-6% | 2%-4% | 4%-6% |
| Ex 773 | 0%-1% | 4%-6% | 4%-6% | 2%-4% | 6%-8% |
| Ex 774 | 0%-1% | 4%-6% | 4%-6% | 2%-4% | 8%-10% |
| Ex 775 | 0%-1% | 4%-6% | 4%-6% | 4%-6% | 0%-1% |
| Ex 776 | 0%-1% | 4%-6% | 4%-6% | 4%-6% | 1%-2% |
| Ex 777 | 0%-1% | 4%-6% | 4%-6% | 4%-6% | 2%-4% |
| Ex 778 | 0%-1% | 4%-6% | 4%-6% | 4%-6% | 4%-6% |
| Ex 779 | 0%-1% | 4%-6% | 4%-6% | 4%-6% | 6%-8% |
| Ex 780 | 0%-1% | 4%-6% | 4%-6% | 4%-6% | 8%-10% |
| Ex 781 | 0%-1% | 4%-6% | 4%-6% | 6%-8% | 0%-1% |
| Ex 782 | 0%-1% | 4%-6% | 4%-6% | 6%-8% | 1%-2% |
| Ex 783 | 0%-1% | 4%-6% | 4%-6% | 6%-8% | 2%-4% |
| Ex 784 | 0%-1% | 4%-6% | 4%-6% | 6%-8% | 4%-6% |
| Ex 785 | 0%-1% | 4%-6% | 4%-6% | 6%-8% | 6%-8% |
| Ex 786 | 0%-1% | 4%-6% | 4%-6% | 6%-8% | 8%-10% |
| Ex 787 | 0%-1% | 4%-6% | 4%-6% | 8%-10% | 0%-1% |
| Ex 788 | 0%-1% | 4%-6% | 4%-6% | 8%-10% | 1%-2% |
| Ex 789 | 0%-1% | 4%-6% | 4%-6% | 8%-10% | 2%-4% |
| Ex 790 | 0%-1% | 4%-6% | 4%-6% | 8%-10% | 4%-6% |
| Ex 791 | 0%-1% | 4%-6% | 4%-6% | 8%-10% | 6%-8% |
| Ex 792 | 0%-1% | 4%-6% | 4%-6% | 8%-10% | 8%-10% |
| Ex 793 | 0%-1% | 4%-6% | 6%-8% | 0%-1% | 0%-1% |
| Ex 794 | 0%-1% | 4%-6% | 6%-8% | 0%-1% | 1%-2% |
| Ex 795 | 0%-1% | 4%-6% | 6%-8% | 0%-1% | 2%-4% |
| Ex 796 | 0%-1% | 4%-6% | 6%-8% | 0%-1% | 4%-6% |
| Ex 797 | 0%-1% | 4%-6% | 6%-8% | 0%-1% | 6%-8% |
| Ex 798 | 0%-1% | 4%-6% | 6%-8% | 0%-1% | 8%-10% |
| Ex 799 | 0%-1% | 4%-6% | 6%-8% | 1%-2% | 0%-1% |
| Ex 800 | 0%-1% | 4%-6% | 6%-8% | 1%-2% | 1%-2% |
| Ex 801 | 0%-1% | 4%-6% | 6%-8% | 1%-2% | 2%-4% |
| Ex 802 | 0%-1% | 4%-6% | 6%-8% | 1%-2% | 4%-6% |
| Ex 803 | 0%-1% | 4%-6% | 6%-8% | 1%-2% | 6%-8% |
| Ex 804 | 0%-1% | 4%-6% | 6%-8% | 1%-2% | 8%-10% |
| Ex 805 | 0%-1% | 4%-6% | 6%-8% | 2%-4% | 0%-1% |
| Ex 806 | 0%-1% | 4%-6% | 6%-8% | 2%-4% | 1%-2% |
| Ex 807 | 0%-1% | 4%-6% | 6%-8% | 2%-4% | 2%-4% |
| Ex 808 | 0%-1% | 4%-6% | 6%-8% | 2%-4% | 4%-6% |
| Ex 809 | 0%-1% | 4%-6% | 6%-8% | 2%-4% | 6%-8% |
| Ex 810 | 0%-1% | 4%-6% | 6%-8% | 2%-4% | 8%-10% |
| Ex 811 | 0%-1% | 4%-6% | 6%-8% | 4%-6% | 0%-1% |
| Ex 812 | 0%-1% | 4%-6% | 6%-8% | 4%-6% | 1%-2% |
| Ex 813 | 0%-1% | 4%-6% | 6%-8% | 4%-6% | 2%-4% |
| Ex 814 | 0%-1% | 4%-6% | 6%-8% | 4%-6% | 4%-6% |
| Ex 815 | 0%-1% | 4%-6% | 6%-8% | 4%-6% | 6%-8% |
| Ex 816 | 0%-1% | 4%-6% | 6%-8% | 4%-6% | 8%-10% |
| Ex 817 | 0%-1% | 4%-6% | 6%-8% | 6%-8% | 0%-1% |
| Ex 818 | 0%-1% | 4%-6% | 6%-8% | 6%-8% | 1%-2% |
| Ex 819 | 0%-1% | 4%-6% | 6%-8% | 6%-8% | 2%-4% |
| Ex 820 | 0%-1% | 4%-6% | 6%-8% | 6%-8% | 4%-6% |
| Ex 821 | 0%-1% | 4%-6% | 6%-8% | 6%-8% | 6%-8% |
| Ex 822 | 0%-1% | 4%-6% | 6%-8% | 6%-8% | 8%-10% |
| Ex 823 | 0%-1% | 4%-6% | 6%-8% | 8%-10% | 0%-1% |
| Ex 824 | 0%-1% | 4%-6% | 6%-8% | 8%-10% | 1%-2% |
| Ex 825 | 0%-1% | 4%-6% | 6%-8% | 8%-10% | 2%-4% |
| Ex 826 | 0%-1% | 4%-6% | 6%-8% | 8%-10% | 4%-6% |
| Ex 827 | 0%-1% | 4%-6% | 6%-8% | 8%-10% | 6%-8% |
| Ex 828 | 0%-1% | 4%-6% | 6%-8% | 8%-10% | 8%-10% |
| Ex 829 | 0%-1% | 4%-6% | 8%-10% | 0%-1% | 0%-1% |
| Ex 830 | 0%-1% | 4%-6% | 8%-10% | 0%-1% | 1%-2% |
| Ex 831 | 0%-1% | 4%-6% | 8%-10% | 0%-1% | 2%-4% |
| Ex 832 | 0%-1% | 4%-6% | 8%-10% | 0%-1% | 4%-6% |
| Ex 833 | 0%-1% | 4%-6% | 8%-10% | 0%-1% | 6%-8% |
| Ex 834 | 0%-1% | 4%-6% | 8%-10% | 0%-1% | 8%-10% |
| Ex 835 | 0%-1% | 4%-6% | 8%-10% | 1%-2% | 0%-1% |
| Ex 836 | 0%-1% | 4%-6% | 8%-10% | 1%-2% | 1%-2% |
| Ex 837 | 0%-1% | 4%-6% | 8%-10% | 1%-2% | 2%-4% |
| Ex 838 | 0%-1% | 4%-6% | 8%-10% | 1%-2% | 4%-6% |
| Ex 839 | 0%-1% | 4%-6% | 8%-10% | 1%-2% | 6%-8% |
| Ex 840 | 0%-1% | 4%-6% | 8%-10% | 1%-2% | 8%-10% |
| Ex 841 | 0%-1% | 4%-6% | 8%-10% | 2%-4% | 0%-1% |
| Ex 842 | 0%-1% | 4%-6% | 8%-10% | 2%-4% | 1%-2% |
| Ex 843 | 0%-1% | 4%-6% | 8%-10% | 2%-4% | 2%-4% |
| Ex 844 | 0%-1% | 4%-6% | 8%-10% | 2%-4% | 4%-6% |
| Ex 845 | 0%-1% | 4%-6% | 8%-10% | 2%-4% | 6%-8% |
| Ex 846 | 0%-1% | 4%-6% | 8%-10% | 2%-4% | 8%-10% |
| Ex 847 | 0%-1% | 4%-6% | 8%-10% | 4%-6% | 0%-1% |
| Ex 848 | 0%-1% | 4%-6% | 8%-10% | 4%-6% | 1%-2% |
| Ex 849 | 0%-1% | 4%-6% | 8%-10% | 4%-6% | 2%-4% |
| Ex 850 | 0%-1% | 4%-6% | 8%-10% | 4%-6% | 4%-6% |
| Ex 851 | 0%-1% | 4%-6% | 8%-10% | 4%-6% | 6%-8% |
| Ex 852 | 0%-1% | 4%-6% | 8%-10% | 4%-6% | 8%-10% |
| Ex 853 | 0%-1% | 4%-6% | 8%-10% | 6%-8% | 0%-1% |
| Ex 854 | 0%-1% | 4%-6% | 8%-10% | 6%-8% | 1%-2% |
| Ex 855 | 0%-1% | 4%-6% | 8%-10% | 6%-8% | 2%-4% |
| Ex 856 | 0%-1% | 4%-6% | 8%-10% | 6%-8% | 4%-6% |
| Ex 857 | 0%-1% | 4%-6% | 8%-10% | 6%-8% | 6%-8% |
| Ex 858 | 0%-1% | 4%-6% | 8%-10% | 6%-8% | 8%-10% |
| Ex 859 | 0%-1% | 4%-6% | 8%-10% | 8%-10% | 0%-1% |
| Ex 860 | 0%-1% | 4%-6% | 8%-10% | 8%-10% | 1%-2% |
| Ex 861 | 0%-1% | 4%-6% | 8%-10% | 8%-10% | 2%-4% |
| Ex 862 | 0%-1% | 4%-6% | 8%-10% | 8%-10% | 4%-6% |
| Ex 863 | 0%-1% | 4%-6% | 8%-10% | 8%-10% | 6%-8% |
| Ex 864 | 0%-1% | 4%-6% | 8%-10% | 8%-10% | 8%-10% |
| Ex 865 | 0%-1% | 6%-8% | 0%-1% | 0%-1% | 0%-1% |
| Ex 866 | 0%-1% | 6%-8% | 0%-1% | 0%-1% | 1%-2% |
| Ex 867 | 0%-1% | 6%-8% | 0%-1% | 0%-1% | 2%-4% |
| Ex 868 | 0%-1% | 6%-8% | 0%-1% | 0%-1% | 4%-6% |
| Ex 869 | 0%-1% | 6%-8% | 0%-1% | 0%-1% | 6%-8% |
| Ex 870 | 0%-1% | 6%-8% | 0%-1% | 0%-1% | 8%-10% |
| Ex 871 | 0%-1% | 6%-8% | 0%-1% | 1%-2% | 0%-1% |
| Ex 872 | 0%-1% | 6%-8% | 0%-1% | 1%-2% | 1%-2% |
| Ex 873 | 0%-1% | 6%-8% | 0%-1% | 1%-2% | 2%-4% |
| Ex 874 | 0%-1% | 6%-8% | 0%-1% | 1%-2% | 4%-6% |
| Ex 875 | 0%-1% | 6%-8% | 0%-1% | 1%-2% | 6%-8% |
| Ex 876 | 0%-1% | 6%-8% | 0%-1% | 1%-2% | 8%-10% |
| Ex 877 | 0%-1% | 6%-8% | 0%-1% | 2%-4% | 0%-1% |
| Ex 878 | 0%-1% | 6%-8% | 0%-1% | 2%-4% | 1%-2% |
| Ex 879 | 0%-1% | 6%-8% | 0%-1% | 2%-4% | 2%-4% |
| Ex 880 | 0%-1% | 6%-8% | 0%-1% | 2%-4% | 4%-6% |
| Ex 881 | 0%-1% | 6%-8% | 0%-1% | 2%-4% | 6%-8% |
| Ex 882 | 0%-1% | 6%-8% | 0%-1% | 2%-4% | 8%-10% |
| Ex 883 | 0%-1% | 6%-8% | 0%-1% | 4%-6% | 0%-1% |
| Ex 884 | 0%-1% | 6%-8% | 0%-1% | 4%-6% | 1%-2% |
| Ex 885 | 0%-1% | 6%-8% | 0%-1% | 4%-6% | 2%-4% |
| Ex 886 | 0%-1% | 6%-8% | 0%-1% | 4%-6% | 4%-6% |
| Ex 887 | 0%-1% | 6%-8% | 0%-1% | 4%-6% | 6%-8% |
| Ex 888 | 0%-1% | 6%-8% | 0%-1% | 4%-6% | 8%-10% |
| Ex 889 | 0%-1% | 6%-8% | 0%-1% | 6%-8% | 0%-1% |
| Ex 890 | 0%-1% | 6%-8% | 0%-1% | 6%-8% | 1%-2% |
| Ex 891 | 0%-1% | 6%-8% | 0%-1% | 6%-8% | 2%-4% |
| Ex 892 | 0%-1% | 6%-8% | 0%-1% | 6%-8% | 4%-6% |
| Ex 893 | 0%-1% | 6%-8% | 0%-1% | 6%-8% | 6%-8% |
| Ex 894 | 0%-1% | 6%-8% | 0%-1% | 6%-8% | 8%-10% |
| Ex 895 | 0%-1% | 6%-8% | 0%-1% | 8%-10% | 0%-1% |
| Ex 896 | 0%-1% | 6%-8% | 0%-1% | 8%-10% | 1%-2% |
| Ex 897 | 0%-1% | 6%-8% | 0%-1% | 8%-10% | 2%-4% |
| Ex 898 | 0%-1% | 6%-8% | 0%-1% | 8%-10% | 4%-6% |
| Ex 899 | 0%-1% | 6%-8% | 0%-1% | 8%-10% | 6%-8% |
| Ex 900 | 0%-1% | 6%-8% | 0%-1% | 8%-10% | 8%-10% |
| Ex 901 | 0%-1% | 6%-8% | 1%-2% | 0%-1% | 0%-1% |
| Ex 902 | 0%-1% | 6%-8% | 1%-2% | 0%-1% | 1%-2% |
| Ex 903 | 0%-1% | 6%-8% | 1%-2% | 0%-1% | 2%-4% |
| Ex 904 | 0%-1% | 6%-8% | 1%-2% | 0%-1% | 4%-6% |
| Ex 905 | 0%-1% | 6%-8% | 1%-2% | 0%-1% | 6%-8% |
| Ex 906 | 0%-1% | 6%-8% | 1%-2% | 0%-1% | 8%-10% |
| Ex 907 | 0%-1% | 6%-8% | 1%-2% | 1%-2% | 0%-1% |
| Ex 908 | 0%-1% | 6%-8% | 1%-2% | 1%-2% | 1%-2% |
| Ex 909 | 0%-1% | 6%-8% | 1%-2% | 1%-2% | 2%-4% |
| Ex 910 | 0%-1% | 6%-8% | 1%-2% | 1%-2% | 4%-6% |
| Ex 911 | 0%-1% | 6%-8% | 1%-2% | 1%-2% | 6%-8% |
| Ex 912 | 0%-1% | 6%-8% | 1%-2% | 1%-2% | 8%-10% |
| Ex 913 | 0%-1% | 6%-8% | 1%-2% | 2%-4% | 0%-1% |
| Ex 914 | 0%-1% | 6%-8% | 1%-2% | 2%-4% | 1%-2% |
| Ex 915 | 0%-1% | 6%-8% | 1%-2% | 2%-4% | 2%-4% |
| Ex 916 | 0%-1% | 6%-8% | 1%-2% | 2%-4% | 4%-6% |
| Ex 917 | 0%-1% | 6%-8% | 1%-2% | 2%-4% | 6%-8% |
| Ex 918 | 0%-1% | 6%-8% | 1%-2% | 2%-4% | 8%-10% |
| Ex 919 | 0%-1% | 6%-8% | 1%-2% | 4%-6% | 0%-1% |
| Ex 920 | 0%-1% | 6%-8% | 1%-2% | 4%-6% | 1%-2% |
| Ex 921 | 0%-1% | 6%-8% | 1%-2% | 4%-6% | 2%-4% |
| Ex 922 | 0%-1% | 6%-8% | 1%-2% | 4%-6% | 4%-6% |
| Ex 923 | 0%-1% | 6%-8% | 1%-2% | 4%-6% | 6%-8% |
| Ex 924 | 0%-1% | 6%-8% | 1%-2% | 4%-6% | 8%-10% |
| Ex 925 | 0%-1% | 6%-8% | 1%-2% | 6%-8% | 0%-1% |
| Ex 926 | 0%-1% | 6%-8% | 1%-2% | 6%-8% | 1%-2% |
| Ex 927 | 0%-1% | 6%-8% | 1%-2% | 6%-8% | 2%-4% |
| Ex 928 | 0%-1% | 6%-8% | 1%-2% | 6%-8% | 4%-6% |
| Ex 929 | 0%-1% | 6%-8% | 1%-2% | 6%-8% | 6%-8% |
| Ex 930 | 0%-1% | 6%-8% | 1%-2% | 6%-8% | 8%-10% |
| Ex 931 | 0%-1% | 6%-8% | 1%-2% | 8%-10% | 0%-1% |
| Ex 932 | 0%-1% | 6%-8% | 1%-2% | 8%-10% | 1%-2% |
| Ex 933 | 0%-1% | 6%-8% | 1%-2% | 8%-10% | 2%-4% |
| Ex 934 | 0%-1% | 6%-8% | 1%-2% | 8%-10% | 4%-6% |
| Ex 935 | 0%-1% | 6%-8% | 1%-2% | 8%-10% | 6%-8% |
| Ex 936 | 0%-1% | 6%-8% | 1%-2% | 8%-10% | 8%-10% |
| Ex 937 | 0%-1% | 6%-8% | 2%-4% | 0%-1% | 0%-1% |
| Ex 938 | 0%-1% | 6%-8% | 2%-4% | 0%-1% | 1%-2% |
| Ex 939 | 0%-1% | 6%-8% | 2%-4% | 0%-1% | 2%-4% |
| Ex 940 | 0%-1% | 6%-8% | 2%-4% | 0%-1% | 4%-6% |
| Ex 941 | 0%-1% | 6%-8% | 2%-4% | 0%-1% | 6%-8% |
| Ex 942 | 0%-1% | 6%-8% | 2%-4% | 0%-1% | 8%-10% |
| Ex 943 | 0%-1% | 6%-8% | 2%-4% | 1%-2% | 0%-1% |
| Ex 944 | 0%-1% | 6%-8% | 2%-4% | 1%-2% | 1%-2% |
| Ex 945 | 0%-1% | 6%-8% | 2%-4% | 1%-2% | 2%-4% |
| Ex 946 | 0%-1% | 6%-8% | 2%-4% | 1%-2% | 4%-6% |
| Ex 947 | 0%-1% | 6%-8% | 2%-4% | 1%-2% | 6%-8% |
| Ex 948 | 0%-1% | 6%-8% | 2%-4% | 1%-2% | 8%-10% |
| Ex 949 | 0%-1% | 6%-8% | 2%-4% | 2%-4% | 0%-1% |
| Ex 950 | 0%-1% | 6%-8% | 2%-4% | 2%-4% | 1%-2% |
| Ex 951 | 0%-1% | 6%-8% | 2%-4% | 2%-4% | 2%-4% |
| Ex 952 | 0%-1% | 6%-8% | 2%-4% | 2%-4% | 4%-6% |
| Ex 953 | 0%-1% | 6%-8% | 2%-4% | 2%-4% | 6%-8% |
| Ex 954 | 0%-1% | 6%-8% | 2%-4% | 2%-4% | 8%-10% |
| Ex 955 | 0%-1% | 6%-8% | 2%-4% | 4%-6% | 0%-1% |
| Ex 956 | 0%-1% | 6%-8% | 2%-4% | 4%-6% | 1%-2% |
| Ex 957 | 0%-1% | 6%-8% | 2%-4% | 4%-6% | 2%-4% |
| Ex 958 | 0%-1% | 6%-8% | 2%-4% | 4%-6% | 4%-6% |
| Ex 959 | 0%-1% | 6%-8% | 2%-4% | 4%-6% | 6%-8% |
| Ex 960 | 0%-1% | 6%-8% | 2%-4% | 4%-6% | 8%-10% |
| Ex 961 | 0%-1% | 6%-8% | 2%-4% | 6%-8% | 0%-1% |
| Ex 962 | 0%-1% | 6%-8% | 2%-4% | 6%-8% | 1%-2% |
| Ex 963 | 0%-1% | 6%-8% | 2%-4% | 6%-8% | 2%-4% |
| Ex 964 | 0%-1% | 6%-8% | 2%-4% | 6%-8% | 4%-6% |
| Ex 965 | 0%-1% | 6%-8% | 2%-4% | 6%-8% | 6%-8% |
| Ex 966 | 0%-1% | 6%-8% | 2%-4% | 6%-8% | 8%-10% |
| Ex 967 | 0%-1% | 6%-8% | 2%-4% | 8%-10% | 0%-1% |
| Ex 968 | 0%-1% | 6%-8% | 2%-4% | 8%-10% | 1%-2% |
| Ex 969 | 0%-1% | 6%-8% | 2%-4% | 8%-10% | 2%-4% |
| Ex 970 | 0%-1% | 6%-8% | 2%-4% | 8%-10% | 4%-6% |
| Ex 971 | 0%-1% | 6%-8% | 2%-4% | 8%-10% | 6%-8% |
| Ex 972 | 0%-1% | 6%-8% | 2%-4% | 8%-10% | 8%-10% |
| Ex 973 | 0%-1% | 6%-8% | 4%-6% | 0%-1% | 0%-1% |
| Ex 974 | 0%-1% | 6%-8% | 4%-6% | 0%-1% | 1%-2% |
| Ex 975 | 0%-1% | 6%-8% | 4%-6% | 0%-1% | 2%-4% |
| Ex 976 | 0%-1% | 6%-8% | 4%-6% | 0%-1% | 4%-6% |
| Ex 977 | 0%-1% | 6%-8% | 4%-6% | 0%-1% | 6%-8% |
| Ex 978 | 0%-1% | 6%-8% | 4%-6% | 0%-1% | 8%-10% |
| Ex 979 | 0%-1% | 6%-8% | 4%-6% | 1%-2% | 0%-1% |
| Ex 980 | 0%-1% | 6%-8% | 4%-6% | 1%-2% | 1%-2% |
| Ex 981 | 0%-1% | 6%-8% | 4%-6% | 1%-2% | 2%-4% |
| Ex 982 | 0%-1% | 6%-8% | 4%-6% | 1%-2% | 4%-6% |
| Ex 983 | 0%-1% | 6%-8% | 4%-6% | 1%-2% | 6%-8% |
| Ex 984 | 0%-1% | 6%-8% | 4%-6% | 1%-2% | 8%-10% |
| Ex 985 | 0%-1% | 6%-8% | 4%-6% | 2%-4% | 0%-1% |
| Ex 986 | 0%-1% | 6%-8% | 4%-6% | 2%-4% | 1%-2% |
| Ex 987 | 0%-1% | 6%-8% | 4%-6% | 2%-4% | 2%-4% |
| Ex 988 | 0%-1% | 6%-8% | 4%-6% | 2%-4% | 4%-6% |
| Ex 989 | 0%-1% | 6%-8% | 4%-6% | 2%-4% | 6%-8% |
| Ex 990 | 0%-1% | 6%-8% | 4%-6% | 2%-4% | 8%-10% |
| Ex 991 | 0%-1% | 6%-8% | 4%-6% | 4%-6% | 0%-1% |
| Ex 992 | 0%-1% | 6%-8% | 4%-6% | 4%-6% | 1%-2% |
| Ex 993 | 0%-1% | 6%-8% | 4%-6% | 4%-6% | 2%-4% |
| Ex 994 | 0%-1% | 6%-8% | 4%-6% | 4%-6% | 4%-6% |
| Ex 995 | 0%-1% | 6%-8% | 4%-6% | 4%-6% | 6%-8% |
| Ex 996 | 0%-1% | 6%-8% | 4%-6% | 4%-6% | 8%-10% |
| Ex 997 | 0%-1% | 6%-8% | 4%-6% | 6%-8% | 0%-1% |
| Ex 998 | 0%-1% | 6%-8% | 4%-6% | 6%-8% | 1%-2% |
| Ex 999 | 0%-1% | 6%-8% | 4%-6% | 6%-8% | 2%-4% |
| Ex 1000 | 0%-1% | 6%-8% | 4%-6% | 6%-8% | 4%-6% |
| Ex 1001 | 0%-1% | 6%-8% | 4%-6% | 6%-8% | 6%-8% |
| Ex 1002 | 0%-1% | 6%-8% | 4%-6% | 6%-8% | 8%-10% |
| Ex 1003 | 0%-1% | 6%-8% | 4%-6% | 8%-10% | 0%-1% |
| Ex 1004 | 0%-1% | 6%-8% | 4%-6% | 8%-10% | 1%-2% |
| Ex 1005 | 0%-1% | 6%-8% | 4%-6% | 8%-10% | 2%-4% |
| Ex 1006 | 0%-1% | 6%-8% | 4%-6% | 8%-10% | 4%-6% |
| Ex 1007 | 0%-1% | 6%-8% | 4%-6% | 8%-10% | 6%-8% |
| Ex 1008 | 0%-1% | 6%-8% | 4%-6% | 8%-10% | 8%-10% |
| Ex 1009 | 0%-1% | 6%-8% | 6%-8% | 0%-1% | 0%-1% |
| Ex 1010 | 0%-1% | 6%-8% | 6%-8% | 0%-1% | 1%-2% |
| Ex 1011 | 0%-1% | 6%-8% | 6%-8% | 0%-1% | 2%-4% |
| Ex 1012 | 0%-1% | 6%-8% | 6%-8% | 0%-1% | 4%-6% |
| Ex 1013 | 0%-1% | 6%-8% | 6%-8% | 0%-1% | 6%-8% |
| Ex 1014 | 0%-1% | 6%-8% | 6%-8% | 0%-1% | 8%-10% |
| Ex 1015 | 0%-1% | 6%-8% | 6%-8% | 1%-2% | 0%-1% |
| Ex 1016 | 0%-1% | 6%-8% | 6%-8% | 1%-2% | 1%-2% |
| Ex 1017 | 0%-1% | 6%-8% | 6%-8% | 1%-2% | 2%-4% |
| Ex 1018 | 0%-1% | 6%-8% | 6%-8% | 1%-2% | 4%-6% |
| Ex 1019 | 0%-1% | 6%-8% | 6%-8% | 1%-2% | 6%-8% |
| Ex 1020 | 0%-1% | 6%-8% | 6%-8% | 1%-2% | 8%-10% |
| Ex 1021 | 0%-1% | 6%-8% | 6%-8% | 2%-4% | 0%-1% |
| Ex 1022 | 0%-1% | 6%-8% | 6%-8% | 2%-4% | 1%-2% |
| Ex 1023 | 0%-1% | 6%-8% | 6%-8% | 2%-4% | 2%-4% |
| Ex 1024 | 0%-1% | 6%-8% | 6%-8% | 2%-4% | 4%-6% |
| Ex 1025 | 0%-1% | 6%-8% | 6%-8% | 2%-4% | 6%-8% |
| Ex 1026 | 0%-1% | 6%-8% | 6%-8% | 2%-4% | 8%-10% |
| Ex 1027 | 0%-1% | 6%-8% | 6%-8% | 4%-6% | 0%-1% |
| Ex 1028 | 0%-1% | 6%-8% | 6%-8% | 4%-6% | 1%-2% |
| Ex 1029 | 0%-1% | 6%-8% | 6%-8% | 4%-6% | 2%-4% |
| Ex 1030 | 0%-1% | 6%-8% | 6%-8% | 4%-6% | 4%-6% |
| Ex 1031 | 0%-1% | 6%-8% | 6%-8% | 4%-6% | 6%-8% |
| Ex 1032 | 0%-1% | 6%-8% | 6%-8% | 4%-6% | 8%-10% |
| Ex 1033 | 0%-1% | 6%-8% | 6%-8% | 6%-8% | 0%-1% |
| Ex 1034 | 0%-1% | 6%-8% | 6%-8% | 6%-8% | 1%-2% |
| Ex 1035 | 0%-1% | 6%-8% | 6%-8% | 6%-8% | 2%-4% |
| Ex 1036 | 0%-1% | 6%-8% | 6%-8% | 6%-8% | 4%-6% |
| Ex 1037 | 0%-1% | 6%-8% | 6%-8% | 6%-8% | 6%-8% |
| Ex 1038 | 0%-1% | 6%-8% | 6%-8% | 6%-8% | 8%-10% |
| Ex 1039 | 0%-1% | 6%-8% | 6%-8% | 8%-10% | 0%-1% |
| Ex 1040 | 0%-1% | 6%-8% | 6%-8% | 8%-10% | 1%-2% |
| Ex 1041 | 0%-1% | 6%-8% | 6%-8% | 8%-10% | 2%-4% |
| Ex 1042 | 0%-1% | 6%-8% | 6%-8% | 8%-10% | 4%-6% |
| Ex 1043 | 0%-1% | 6%-8% | 6%-8% | 8%-10% | 6%-8% |
| Ex 1044 | 0%-1% | 6%-8% | 6%-8% | 8%-10% | 8%-10% |
| Ex 1045 | 0%-1% | 6%-8% | 8%-10% | 0%-1% | 0%-1% |
| Ex 1046 | 0%-1% | 6%-8% | 8%-10% | 0%-1% | 1%-2% |
| Ex 1047 | 0%-1% | 6%-8% | 8%-10% | 0%-1% | 2%-4% |
| Ex 1048 | 0%-1% | 6%-8% | 8%-10% | 0%-1% | 4%-6% |
| Ex 1049 | 0%-1% | 6%-8% | 8%-10% | 0%-1% | 6%-8% |
| Ex 1050 | 0%-1% | 6%-8% | 8%-10% | 0%-1% | 8%-10% |
| Ex 1051 | 0%-1% | 6%-8% | 8%-10% | 1%-2% | 0%-1% |
| Ex 1052 | 0%-1% | 6%-8% | 8%-10% | 1%-2% | 1%-2% |
| Ex 1053 | 0%-1% | 6%-8% | 8%-10% | 1%-2% | 2%-4% |
| Ex 1054 | 0%-1% | 6%-8% | 8%-10% | 1%-2% | 4%-6% |
| Ex 1055 | 0%-1% | 6%-8% | 8%-10% | 1%-2% | 6%-8% |
| Ex 1056 | 0%-1% | 6%-8% | 8%-10% | 1%-2% | 8%-10% |
| Ex 1057 | 0%-1% | 6%-8% | 8%-10% | 2%-4% | 0%-1% |
| Ex 1058 | 0%-1% | 6%-8% | 8%-10% | 2%-4% | 1%-2% |
| Ex 1059 | 0%-1% | 6%-8% | 8%-10% | 2%-4% | 2%-4% |
| Ex 1060 | 0%-1% | 6%-8% | 8%-10% | 2%-4% | 4%-6% |
| Ex 1061 | 0%-1% | 6%-8% | 8%-10% | 2%-4% | 6%-8% |
| Ex 1062 | 0%-1% | 6%-8% | 8%-10% | 2%-4% | 8%-10% |
| Ex 1063 | 0%-1% | 6%-8% | 8%-10% | 4%-6% | 0%-1% |
| Ex 1064 | 0%-1% | 6%-8% | 8%-10% | 4%-6% | 1%-2% |
| Ex 1065 | 0%-1% | 6%-8% | 8%-10% | 4%-6% | 2%-4% |
| Ex 1066 | 0%-1% | 6%-8% | 8%-10% | 4%-6% | 4%-6% |
| Ex 1067 | 0%-1% | 6%-8% | 8%-10% | 4%-6% | 6%-8% |
| Ex 1068 | 0%-1% | 6%-8% | 8%-10% | 4%-6% | 8%-10% |
| Ex 1069 | 0%-1% | 6%-8% | 8%-10% | 6%-8% | 0%-1% |
| Ex 1070 | 0%-1% | 6%-8% | 8%-10% | 6%-8% | 1%-2% |
| Ex 1071 | 0%-1% | 6%-8% | 8%-10% | 6%-8% | 2%-4% |
| Ex 1072 | 0%-1% | 6%-8% | 8%-10% | 6%-8% | 4%-6% |
| Ex 1073 | 0%-1% | 6%-8% | 8%-10% | 6%-8% | 6%-8% |
| Ex 1074 | 0%-1% | 6%-8% | 8%-10% | 6%-8% | 8%-10% |
| Ex 1075 | 0%-1% | 6%-8% | 8%-10% | 8%-10% | 0%-1% |
| Ex 1076 | 0%-1% | 6%-8% | 8%-10% | 8%-10% | 1%-2% |
| Ex 1077 | 0%-1% | 6%-8% | 8%-10% | 8%-10% | 2%-4% |
| Ex 1078 | 0%-1% | 6%-8% | 8%-10% | 8%-10% | 4%-6% |
| Ex 1079 | 0%-1% | 6%-8% | 8%-10% | 8%-10% | 6%-8% |
| Ex 1080 | 0%-1% | 6%-8% | 8%-10% | 8%-10% | 8%-10% |
| Ex 1081 | 0%-1% | 8%-10% | 0%-1% | 0%-1% | 0%-1% |
| Ex 1082 | 0%-1% | 8%-10% | 0%-1% | 0%-1% | 1%-2% |
| Ex 1083 | 0%-1% | 8%-10% | 0%-1% | 0%-1% | 2%-4% |
| Ex 1084 | 0%-1% | 8%-10% | 0%-1% | 0%-1% | 4%-6% |
| Ex 1085 | 0%-1% | 8%-10% | 0%-1% | 0%-1% | 6%-8% |
| Ex 1086 | 0%-1% | 8%-10% | 0%-1% | 0%-1% | 8%-10% |
| Ex 1087 | 0%-1% | 8%-10% | 0%-1% | 1%-2% | 0%-1% |
| Ex 1088 | 0%-1% | 8%-10% | 0%-1% | 1%-2% | 1%-2% |
| Ex 1089 | 0%-1% | 8%-10% | 0%-1% | 1%-2% | 2%-4% |
| Ex 1090 | 0%-1% | 8%-10% | 0%-1% | 1%-2% | 4%-6% |
| Ex 1091 | 0%-1% | 8%-10% | 0%-1% | 1%-2% | 6%-8% |
| Ex 1092 | 0%-1% | 8%-10% | 0%-1% | 1%-2% | 8%-10% |
| Ex 1093 | 0%-1% | 8%-10% | 0%-1% | 2%-4% | 0%-1% |
| Ex 1094 | 0%-1% | 8%-10% | 0%-1% | 2%-4% | 1%-2% |
| Ex 1095 | 0%-1% | 8%-10% | 0%-1% | 2%-4% | 2%-4% |
| Ex 1096 | 0%-1% | 8%-10% | 0%-1% | 2%-4% | 4%-6% |
| Ex 1097 | 0%-1% | 8%-10% | 0%-1% | 2%-4% | 6%-8% |
| Ex 1098 | 0%-1% | 8%-10% | 0%-1% | 2%-4% | 8%-10% |
| Ex 1099 | 0%-1% | 8%-10% | 0%-1% | 4%-6% | 0%-1% |
| Ex 1100 | 0%-1% | 8%-10% | 0%-1% | 4%-6% | 1%-2% |
| Ex 1101 | 0%-1% | 8%-10% | 0%-1% | 4%-6% | 2%-4% |
| Ex 1102 | 0%-1% | 8%-10% | 0%-1% | 4%-6% | 4%-6% |
| Ex 1103 | 0%-1% | 8%-10% | 0%-1% | 4%-6% | 6%-8% |
| Ex 1104 | 0%-1% | 8%-10% | 0%-1% | 4%-6% | 8%-10% |
| Ex 1105 | 0%-1% | 8%-10% | 0%-1% | 6%-8% | 0%-1% |
| Ex 1106 | 0%-1% | 8%-10% | 0%-1% | 6%-8% | 1%-2% |
| Ex 1107 | 0%-1% | 8%-10% | 0%-1% | 6%-8% | 2%-4% |
| Ex 1108 | 0%-1% | 8%-10% | 0%-1% | 6%-8% | 4%-6% |
| Ex 1109 | 0%-1% | 8%-10% | 0%-1% | 6%-8% | 6%-8% |
| Ex 1110 | 0%-1% | 8%-10% | 0%-1% | 6%-8% | 8%-10% |
| Ex 1111 | 0%-1% | 8%-10% | 0%-1% | 8%-10% | 0%-1% |
| Ex 1112 | 0%-1% | 8%-10% | 0%-1% | 8%-10% | 1%-2% |
| Ex 1113 | 0%-1% | 8%-10% | 0%-1% | 8%-10% | 2%-4% |
| Ex 1114 | 0%-1% | 8%-10% | 0%-1% | 8%-10% | 4%-6% |
| Ex 1115 | 0%-1% | 8%-10% | 0%-1% | 8%-10% | 6%-8% |
| Ex 1116 | 0%-1% | 8%-10% | 0%-1% | 8%-10% | 8%-10% |
| Ex 1117 | 0%-1% | 8%-10% | 1%-2% | 0%-1% | 0%-1% |
| Ex 1118 | 0%-1% | 8%-10% | 1%-2% | 0%-1% | 1%-2% |
| Ex 1119 | 0%-1% | 8%-10% | 1%-2% | 0%-1% | 2%-4% |
| Ex 1120 | 0%-1% | 8%-10% | 1%-2% | 0%-1% | 4%-6% |
| Ex 1121 | 0%-1% | 8%-10% | 1%-2% | 0%-1% | 6%-8% |
| Ex 1122 | 0%-1% | 8%-10% | 1%-2% | 0%-1% | 8%-10% |
| Ex 1123 | 0%-1% | 8%-10% | 1%-2% | 1%-2% | 0%-1% |
| Ex 1124 | 0%-1% | 8%-10% | 1%-2% | 1%-2% | 1%-2% |
| Ex 1125 | 0%-1% | 8%-10% | 1%-2% | 1%-2% | 2%-4% |
| Ex 1126 | 0%-1% | 8%-10% | 1%-2% | 1%-2% | 4%-6% |
| Ex 1127 | 0%-1% | 8%-10% | 1%-2% | 1%-2% | 6%-8% |
| Ex 1128 | 0%-1% | 8%-10% | 1%-2% | 1%-2% | 8%-10% |
| Ex 1129 | 0%-1% | 8%-10% | 1%-2% | 2%-4% | 0%-1% |
| Ex 1130 | 0%-1% | 8%-10% | 1%-2% | 2%-4% | 1%-2% |
| Ex 1131 | 0%-1% | 8%-10% | 1%-2% | 2%-4% | 2%-4% |
| Ex 1132 | 0%-1% | 8%-10% | 1%-2% | 2%-4% | 4%-6% |
| Ex 1133 | 0%-1% | 8%-10% | 1%-2% | 2%-4% | 6%-8% |
| Ex 1134 | 0%-1% | 8%-10% | 1%-2% | 2%-4% | 8%-10% |
| Ex 1135 | 0%-1% | 8%-10% | 1%-2% | 4%-6% | 0%-1% |
| Ex 1136 | 0%-1% | 8%-10% | 1%-2% | 4%-6% | 1%-2% |
| Ex 1137 | 0%-1% | 8%-10% | 1%-2% | 4%-6% | 2%-4% |
| Ex 1138 | 0%-1% | 8%-10% | 1%-2% | 4%-6% | 4%-6% |
| Ex 1139 | 0%-1% | 8%-10% | 1%-2% | 4%-6% | 6%-8% |
| Ex 1140 | 0%-1% | 8%-10% | 1%-2% | 4%-6% | 8%-10% |
| Ex 1141 | 0%-1% | 8%-10% | 1%-2% | 6%-8% | 0%-1% |
| Ex 1142 | 0%-1% | 8%-10% | 1%-2% | 6%-8% | 1%-2% |
| Ex 1143 | 0%-1% | 8%-10% | 1%-2% | 6%-8% | 2%-4% |
| Ex 1144 | 0%-1% | 8%-10% | 1%-2% | 6%-8% | 4%-6% |
| Ex 1145 | 0%-1% | 8%-10% | 1%-2% | 6%-8% | 6%-8% |
| Ex 1146 | 0%-1% | 8%-10% | 1%-2% | 6%-8% | 8%-10% |
| Ex 1147 | 0%-1% | 8%-10% | 1%-2% | 8%-10% | 0%-1% |
| Ex 1148 | 0%-1% | 8%-10% | 1%-2% | 8%-10% | 1%-2% |
| Ex 1149 | 0%-1% | 8%-10% | 1%-2% | 8%-10% | 2%-4% |
| Ex 1150 | 0%-1% | 8%-10% | 1%-2% | 8%-10% | 4%-6% |
| Ex 1151 | 0%-1% | 8%-10% | 1%-2% | 8%-10% | 6%-8% |
| Ex 1152 | 0%-1% | 8%-10% | 1%-2% | 8%-10% | 8%-10% |
| Ex 1153 | 0%-1% | 8%-10% | 2%-4% | 0%-1% | 0%-1% |
| Ex 1154 | 0%-1% | 8%-10% | 2%-4% | 0%-1% | 1%-2% |
| Ex 1155 | 0%-1% | 8%-10% | 2%-4% | 0%-1% | 2%-4% |
| Ex 1156 | 0%-1% | 8%-10% | 2%-4% | 0%-1% | 4%-6% |
| Ex 1157 | 0%-1% | 8%-10% | 2%-4% | 0%-1% | 6%-8% |
| Ex 1158 | 0%-1% | 8%-10% | 2%-4% | 0%-1% | 8%-10% |
| Ex 1159 | 0%-1% | 8%-10% | 2%-4% | 1%-2% | 0%-1% |
| Ex 1160 | 0%-1% | 8%-10% | 2%-4% | 1%-2% | 1%-2% |
| Ex 1161 | 0%-1% | 8%-10% | 2%-4% | 1%-2% | 2%-4% |
| Ex 1162 | 0%-1% | 8%-10% | 2%-4% | 1%-2% | 4%-6% |
| Ex 1163 | 0%-1% | 8%-10% | 2%-4% | 1%-2% | 6%-8% |
| Ex 1164 | 0%-1% | 8%-10% | 2%-4% | 1%-2% | 8%-10% |
| Ex 1165 | 0%-1% | 8%-10% | 2%-4% | 2%-4% | 0%-1% |
| Ex 1166 | 0%-1% | 8%-10% | 2%-4% | 2%-4% | 1%-2% |
| Ex 1167 | 0%-1% | 8%-10% | 2%-4% | 2%-4% | 2%-4% |
| Ex 1168 | 0%-1% | 8%-10% | 2%-4% | 2%-4% | 4%-6% |
| Ex 1169 | 0%-1% | 8%-10% | 2%-4% | 2%-4% | 6%-8% |
| Ex 1170 | 0%-1% | 8%-10% | 2%-4% | 2%-4% | 8%-10% |
| Ex 1171 | 0%-1% | 8%-10% | 2%-4% | 4%-6% | 0%-1% |
| Ex 1172 | 0%-1% | 8%-10% | 2%-4% | 4%-6% | 1%-2% |
| Ex 1173 | 0%-1% | 8%-10% | 2%-4% | 4%-6% | 2%-4% |
| Ex 1174 | 0%-1% | 8%-10% | 2%-4% | 4%-6% | 4%-6% |
| Ex 1175 | 0%-1% | 8%-10% | 2%-4% | 4%-6% | 6%-8% |
| Ex 1176 | 0%-1% | 8%-10% | 2%-4% | 4%-6% | 8%-10% |
| Ex 1177 | 0%-1% | 8%-10% | 2%-4% | 6%-8% | 0%-1% |
| Ex 1178 | 0%-1% | 8%-10% | 2%-4% | 6%-8% | 1%-2% |
| Ex 1179 | 0%-1% | 8%-10% | 2%-4% | 6%-8% | 2%-4% |
| Ex 1180 | 0%-1% | 8%-10% | 2%-4% | 6%-8% | 4%-6% |
| Ex 1181 | 0%-1% | 8%-10% | 2%-4% | 6%-8% | 6%-8% |
| Ex 1182 | 0%-1% | 8%-10% | 2%-4% | 6%-8% | 8%-10% |
| Ex 1183 | 0%-1% | 8%-10% | 2%-4% | 8%-10% | 0%-1% |
| Ex 1184 | 0%-1% | 8%-10% | 2%-4% | 8%-10% | 1%-2% |
| Ex 1185 | 0%-1% | 8%-10% | 2%-4% | 8%-10% | 2%-4% |
| Ex 1186 | 0%-1% | 8%-10% | 2%-4% | 8%-10% | 4%-6% |
| Ex 1187 | 0%-1% | 8%-10% | 2%-4% | 8%-10% | 6%-8% |
| Ex 1188 | 0%-1% | 8%-10% | 2%-4% | 8%-10% | 8%-10% |
| Ex 1189 | 0%-1% | 8%-10% | 4%-6% | 0%-1% | 0%-1% |
| Ex 1190 | 0%-1% | 8%-10% | 4%-6% | 0%-1% | 1%-2% |
| Ex 1191 | 0%-1% | 8%-10% | 4%-6% | 0%-1% | 2%-4% |
| Ex 1192 | 0%-1% | 8%-10% | 4%-6% | 0%-1% | 4%-6% |
| Ex 1193 | 0%-1% | 8%-10% | 4%-6% | 0%-1% | 6%-8% |
| Ex 1194 | 0%-1% | 8%-10% | 4%-6% | 0%-1% | 8%-10% |
| Ex 1195 | 0%-1% | 8%-10% | 4%-6% | 1%-2% | 0%-1% |
| Ex 1196 | 0%-1% | 8%-10% | 4%-6% | 1%-2% | 1%-2% |
| Ex 1197 | 0%-1% | 8%-10% | 4%-6% | 1%-2% | 2%-4% |
| Ex 1198 | 0%-1% | 8%-10% | 4%-6% | 1%-2% | 4%-6% |
| Ex 1199 | 0%-1% | 8%-10% | 4%-6% | 1%-2% | 6%-8% |
| Ex 1200 | 0%-1% | 8%-10% | 4%-6% | 1%-2% | 8%-10% |
| Ex 1201 | 0%-1% | 8%-10% | 4%-6% | 2%-4% | 0%-1% |
| Ex 1202 | 0%-1% | 8%-10% | 4%-6% | 2%-4% | 1%-2% |
| Ex 1203 | 0%-1% | 8%-10% | 4%-6% | 2%-4% | 2%-4% |
| Ex 1204 | 0%-1% | 8%-10% | 4%-6% | 2%-4% | 4%-6% |
| Ex 1205 | 0%-1% | 8%-10% | 4%-6% | 2%-4% | 6%-8% |
| Ex 1206 | 0%-1% | 8%-10% | 4%-6% | 2%-4% | 8%-10% |
| Ex 1207 | 0%-1% | 8%-10% | 4%-6% | 4%-6% | 0%-1% |
| Ex 1208 | 0%-1% | 8%-10% | 4%-6% | 4%-6% | 1%-2% |
| Ex 1209 | 0%-1% | 8%-10% | 4%-6% | 4%-6% | 2%-4% |
| Ex 1210 | 0%-1% | 8%-10% | 4%-6% | 4%-6% | 4%-6% |
| Ex 1211 | 0%-1% | 8%-10% | 4%-6% | 4%-6% | 6%-8% |
| Ex 1212 | 0%-1% | 8%-10% | 4%-6% | 4%-6% | 8%-10% |
| Ex 1213 | 0%-1% | 8%-10% | 4%-6% | 6%-8% | 0%-1% |
| Ex 1214 | 0%-1% | 8%-10% | 4%-6% | 6%-8% | 1%-2% |
| Ex 1215 | 0%-1% | 8%-10% | 4%-6% | 6%-8% | 2%-4% |
| Ex 1216 | 0%-1% | 8%-10% | 4%-6% | 6%-8% | 4%-6% |
| Ex 1217 | 0%-1% | 8%-10% | 4%-6% | 6%-8% | 6%-8% |
| Ex 1218 | 0%-1% | 8%-10% | 4%-6% | 6%-8% | 8%-10% |
| Ex 1219 | 0%-1% | 8%-10% | 4%-6% | 8%-10% | 0%-1% |
| Ex 1220 | 0%-1% | 8%-10% | 4%-6% | 8%-10% | 1%-2% |
| Ex 1221 | 0%-1% | 8%-10% | 4%-6% | 8%-10% | 2%-4% |
| Ex 1222 | 0%-1% | 8%-10% | 4%-6% | 8%-10% | 4%-6% |
| Ex 1223 | 0%-1% | 8%-10% | 4%-6% | 8%-10% | 6%-8% |
| Ex 1224 | 0%-1% | 8%-10% | 4%-6% | 8%-10% | 8%-10% |
| Ex 1225 | 0%-1% | 8%-10% | 6%-8% | 0%-1% | 0%-1% |
| Ex 1226 | 0%-1% | 8%-10% | 6%-8% | 0%-1% | 1%-2% |
| Ex 1227 | 0%-1% | 8%-10% | 6%-8% | 0%-1% | 2%-4% |
| Ex 1228 | 0%-1% | 8%-10% | 6%-8% | 0%-1% | 4%-6% |
| Ex 1229 | 0%-1% | 8%-10% | 6%-8% | 0%-1% | 6%-8% |
| Ex 1230 | 0%-1% | 8%-10% | 6%-8% | 0%-1% | 8%-10% |
| Ex 1231 | 0%-1% | 8%-10% | 6%-8% | 1%-2% | 0%-1% |
| Ex 1232 | 0%-1% | 8%-10% | 6%-8% | 1%-2% | 1%-2% |
| Ex 1233 | 0%-1% | 8%-10% | 6%-8% | 1%-2% | 2%-4% |
| Ex 1234 | 0%-1% | 8%-10% | 6%-8% | 1%-2% | 4%-6% |
| Ex 1235 | 0%-1% | 8%-10% | 6%-8% | 1%-2% | 6%-8% |
| Ex 1236 | 0%-1% | 8%-10% | 6%-8% | 1%-2% | 8%-10% |
| Ex 1237 | 0%-1% | 8%-10% | 6%-8% | 2%-4% | 0%-1% |
| Ex 1238 | 0%-1% | 8%-10% | 6%-8% | 2%-4% | 1%-2% |
| Ex 1239 | 0%-1% | 8%-10% | 6%-8% | 2%-4% | 2%-4% |
| Ex 1240 | 0%-1% | 8%-10% | 6%-8% | 2%-4% | 4%-6% |
| Ex 1241 | 0%-1% | 8%-10% | 6%-8% | 2%-4% | 6%-8% |
| Ex 1242 | 0%-1% | 8%-10% | 6%-8% | 2%-4% | 8%-10% |
| Ex 1243 | 0%-1% | 8%-10% | 6%-8% | 4%-6% | 0%-1% |
| Ex 1244 | 0%-1% | 8%-10% | 6%-8% | 4%-6% | 1%-2% |
| Ex 1245 | 0%-1% | 8%-10% | 6%-8% | 4%-6% | 2%-4% |
| Ex 1246 | 0%-1% | 8%-10% | 6%-8% | 4%-6% | 4%-6% |
| Ex 1247 | 0%-1% | 8%-10% | 6%-8% | 4%-6% | 6%-8% |
| Ex 1248 | 0%-1% | 8%-10% | 6%-8% | 4%-6% | 8%-10% |
| Ex 1249 | 0%-1% | 8%-10% | 6%-8% | 6%-8% | 0%-1% |
| Ex 1250 | 0%-1% | 8%-10% | 6%-8% | 6%-8% | 1%-2% |
| Ex 1251 | 0%-1% | 8%-10% | 6%-8% | 6%-8% | 2%-4% |
| Ex 1252 | 0%-1% | 8%-10% | 6%-8% | 6%-8% | 4%-6% |
| Ex 1253 | 0%-1% | 8%-10% | 6%-8% | 6%-8% | 6%-8% |
| Ex 1254 | 0%-1% | 8%-10% | 6%-8% | 6%-8% | 8%-10% |
| Ex 1255 | 0%-1% | 8%-10% | 6%-8% | 8%-10% | 0%-1% |
| Ex 1256 | 0%-1% | 8%-10% | 6%-8% | 8%-10% | 1%-2% |
| Ex 1257 | 0%-1% | 8%-10% | 6%-8% | 8%-10% | 2%-4% |
| Ex 1258 | 0%-1% | 8%-10% | 6%-8% | 8%-10% | 4%-6% |
| Ex 1259 | 0%-1% | 8%-10% | 6%-8% | 8%-10% | 6%-8% |
| Ex 1260 | 0%-1% | 8%-10% | 6%-8% | 8%-10% | 8%-10% |
| Ex 1261 | 0%-1% | 8%-10% | 8%-10% | 0%-1% | 0%-1% |
| Ex 1262 | 0%-1% | 8%-10% | 8%-10% | 0%-1% | 1%-2% |
| Ex 1263 | 0%-1% | 8%-10% | 8%-10% | 0%-1% | 2%-4% |
| Ex 1264 | 0%-1% | 8%-10% | 8%-10% | 0%-1% | 4%-6% |
| Ex 1265 | 0%-1% | 8%-10% | 8%-10% | 0%-1% | 6%-8% |
| Ex 1266 | 0%-1% | 8%-10% | 8%-10% | 0%-1% | 8%-10% |
| Ex 1267 | 0%-1% | 8%-10% | 8%-10% | 1%-2% | 0%-1% |
| Ex 1268 | 0%-1% | 8%-10% | 8%-10% | 1%-2% | 1%-2% |
| Ex 1269 | 0%-1% | 8%-10% | 8%-10% | 1%-2% | 2%-4% |
| Ex 1270 | 0%-1% | 8%-10% | 8%-10% | 1%-2% | 4%-6% |
| Ex 1271 | 0%-1% | 8%-10% | 8%-10% | 1%-2% | 6%-8% |
| Ex 1272 | 0%-1% | 8%-10% | 8%-10% | 1%-2% | 8%-10% |
| Ex 1273 | 0%-1% | 8%-10% | 8%-10% | 2%-4% | 0%-1% |
| Ex 1274 | 0%-1% | 8%-10% | 8%-10% | 2%-4% | 1%-2% |
| Ex 1275 | 0%-1% | 8%-10% | 8%-10% | 2%-4% | 2%-4% |
| Ex 1276 | 0%-1% | 8%-10% | 8%-10% | 2%-4% | 4%-6% |
| Ex 1277 | 0%-1% | 8%-10% | 8%-10% | 2%-4% | 6%-8% |
| Ex 1278 | 0%-1% | 8%-10% | 8%-10% | 2%-4% | 8%-10% |
| Ex 1279 | 0%-1% | 8%-10% | 8%-10% | 4%-6% | 0%-1% |
| Ex 1280 | 0%-1% | 8%-10% | 8%-10% | 4%-6% | 1%-2% |
| Ex 1281 | 0%-1% | 8%-10% | 8%-10% | 4%-6% | 2%-4% |
| Ex 1282 | 0%-1% | 8%-10% | 8%-10% | 4%-6% | 4%-6% |
| Ex 1283 | 0%-1% | 8%-10% | 8%-10% | 4%-6% | 6%-8% |
| Ex 1284 | 0%-1% | 8%-10% | 8%-10% | 4%-6% | 8%-10% |
| Ex 1285 | 0%-1% | 8%-10% | 8%-10% | 6%-8% | 0%-1% |
| Ex 1286 | 0%-1% | 8%-10% | 8%-10% | 6%-8% | 1%-2% |
| Ex 1287 | 0%-1% | 8%-10% | 8%-10% | 6%-8% | 2%-4% |
| Ex 1288 | 0%-1% | 8%-10% | 8%-10% | 6%-8% | 4%-6% |
| Ex 1289 | 0%-1% | 8%-10% | 8%-10% | 6%-8% | 6%-8% |
| Ex 1290 | 0%-1% | 8%-10% | 8%-10% | 6%-8% | 8%-10% |
| Ex 1291 | 0%-1% | 8%-10% | 8%-10% | 8%-10% | 0%-1% |
| Ex 1292 | 0%-1% | 8%-10% | 8%-10% | 8%-10% | 1%-2% |
| Ex 1293 | 0%-1% | 8%-10% | 8%-10% | 8%-10% | 2%-4% |
| Ex 1294 | 0%-1% | 8%-10% | 8%-10% | 8%-10% | 4%-6% |
| Ex 1295 | 0%-1% | 8%-10% | 8%-10% | 8%-10% | 6%-8% |
| Ex 1296 | 0%-1% | 8%-10% | 8%-10% | 8%-10% | 8%-10% |
| Ex 1297 | 1%-2% | 0%-1% | 0%-1% | 0%-1% | 0%-1% |
| Ex 1298 | 1%-2% | 0%-1% | 0%-1% | 0%-1% | 1%-2% |
| Ex 1299 | 1%-2% | 0%-1% | 0%-1% | 0%-1% | 2%-4% |
| Ex 1300 | 1%-2% | 0%-1% | 0%-1% | 0%-1% | 4%-6% |
| Ex 1301 | 1%-2% | 0%-1% | 0%-1% | 0%-1% | 6%-8% |
| Ex 1302 | 1%-2% | 0%-1% | 0%-1% | 0%-1% | 8%-10% |
| Ex 1303 | 1%-2% | 0%-1% | 0%-1% | 1%-2% | 0%-1% |
| Ex 1304 | 1%-2% | 0%-1% | 0%-1% | 1%-2% | 1%-2% |
| Ex 1305 | 1%-2% | 0%-1% | 0%-1% | 1%-2% | 2%-4% |
| Ex 1306 | 1%-2% | 0%-1% | 0%-1% | 1%-2% | 4%-6% |
| Ex 1307 | 1%-2% | 0%-1% | 0%-1% | 1%-2% | 6%-8% |
| Ex 1308 | 1%-2% | 0%-1% | 0%-1% | 1%-2% | 8%-10% |
| Ex 1309 | 1%-2% | 0%-1% | 0%-1% | 2%-4% | 0%-1% |
| Ex 1310 | 1%-2% | 0%-1% | 0%-1% | 2%-4% | 1%-2% |
| Ex 1311 | 1%-2% | 0%-1% | 0%-1% | 2%-4% | 2%-4% |
| Ex 1312 | 1%-2% | 0%-1% | 0%-1% | 2%-4% | 4%-6% |
| Ex 1313 | 1%-2% | 0%-1% | 0%-1% | 2%-4% | 6%-8% |
| Ex 1314 | 1%-2% | 0%-1% | 0%-1% | 2%-4% | 8%-10% |
| Ex 1315 | 1%-2% | 0%-1% | 0%-1% | 4%-6% | 0%-1% |
| Ex 1316 | 1%-2% | 0%-1% | 0%-1% | 4%-6% | 1%-2% |
| Ex 1317 | 1%-2% | 0%-1% | 0%-1% | 4%-6% | 2%-4% |
| Ex 1318 | 1%-2% | 0%-1% | 0%-1% | 4%-6% | 4%-6% |
| Ex 1319 | 1%-2% | 0%-1% | 0%-1% | 4%-6% | 6%-8% |
| Ex 1320 | 1%-2% | 0%-1% | 0%-1% | 4%-6% | 8%-10% |
| Ex 1321 | 1%-2% | 0%-1% | 0%-1% | 6%-8% | 0%-1% |
| Ex 1322 | 1%-2% | 0%-1% | 0%-1% | 6%-8% | 1%-2% |
| Ex 1323 | 1%-2% | 0%-1% | 0%-1% | 6%-8% | 2%-4% |
| Ex 1324 | 1%-2% | 0%-1% | 0%-1% | 6%-8% | 4%-6% |
| Ex 1325 | 1%-2% | 0%-1% | 0%-1% | 6%-8% | 6%-8% |
| Ex 1326 | 1%-2% | 0%-1% | 0%-1% | 6%-8% | 8%-10% |
| Ex 1327 | 1%-2% | 0%-1% | 0%-1% | 8%-10% | 0%-1% |
| Ex 1328 | 1%-2% | 0%-1% | 0%-1% | 8%-10% | 1%-2% |
| Ex 1329 | 1%-2% | 0%-1% | 0%-1% | 8%-10% | 2%-4% |
| Ex 1330 | 1%-2% | 0%-1% | 0%-1% | 8%-10% | 4%-6% |
| Ex 1331 | 1%-2% | 0%-1% | 0%-1% | 8%-10% | 6%-8% |
| Ex 1332 | 1%-2% | 0%-1% | 0%-1% | 8%-10% | 8%-10% |
| Ex 1333 | 1%-2% | 0%-1% | 1%-2% | 0%-1% | 0%-1% |
| Ex 1334 | 1%-2% | 0%-1% | 1%-2% | 0%-1% | 1%-2% |
| Ex 1335 | 1%-2% | 0%-1% | 1%-2% | 0%-1% | 2%-4% |
| Ex 1336 | 1%-2% | 0%-1% | 1%-2% | 0%-1% | 4%-6% |
| Ex 1337 | 1%-2% | 0%-1% | 1%-2% | 0%-1% | 6%-8% |
| Ex 1338 | 1%-2% | 0%-1% | 1%-2% | 0%-1% | 8%-10% |
| Ex 1339 | 1%-2% | 0%-1% | 1%-2% | 1%-2% | 0%-1% |
| Ex 1340 | 1%-2% | 0%-1% | 1%-2% | 1%-2% | 1%-2% |
| Ex 1341 | 1%-2% | 0%-1% | 1%-2% | 1%-2% | 2%-4% |
| Ex 1342 | 1%-2% | 0%-1% | 1%-2% | 1%-2% | 4%-6% |
| Ex 1343 | 1%-2% | 0%-1% | 1%-2% | 1%-2% | 6%-8% |
| Ex 1344 | 1%-2% | 0%-1% | 1%-2% | 1%-2% | 8%-10% |
| Ex 1345 | 1%-2% | 0%-1% | 1%-2% | 2%-4% | 0%-1% |
| Ex 1346 | 1%-2% | 0%-1% | 1%-2% | 2%-4% | 1%-2% |
| Ex 1347 | 1%-2% | 0%-1% | 1%-2% | 2%-4% | 2%-4% |
| Ex 1348 | 1%-2% | 0%-1% | 1%-2% | 2%-4% | 4%-6% |
| Ex 1349 | 1%-2% | 0%-1% | 1%-2% | 2%-4% | 6%-8% |
| Ex 1350 | 1%-2% | 0%-1% | 1%-2% | 2%-4% | 8%-10% |
| Ex 1351 | 1%-2% | 0%-1% | 1%-2% | 4%-6% | 0%-1% |
| Ex 1352 | 1%-2% | 0%-1% | 1%-2% | 4%-6% | 1%-2% |
| Ex 1353 | 1%-2% | 0%-1% | 1%-2% | 4%-6% | 2%-4% |
| Ex 1354 | 1%-2% | 0%-1% | 1%-2% | 4%-6% | 4%-6% |
| Ex 1355 | 1%-2% | 0%-1% | 1%-2% | 4%-6% | 6%-8% |
| Ex 1356 | 1%-2% | 0%-1% | 1%-2% | 4%-6% | 8%-10% |
| Ex 1357 | 1%-2% | 0%-1% | 1%-2% | 6%-8% | 0%-1% |
| Ex 1358 | 1%-2% | 0%-1% | 1%-2% | 6%-8% | 1%-2% |
| Ex 1359 | 1%-2% | 0%-1% | 1%-2% | 6%-8% | 2%-4% |
| Ex 1360 | 1%-2% | 0%-1% | 1%-2% | 6%-8% | 4%-6% |
| Ex 1361 | 1%-2% | 0%-1% | 1%-2% | 6%-8% | 6%-8% |
| Ex 1362 | 1%-2% | 0%-1% | 1%-2% | 6%-8% | 8%-10% |
| Ex 1363 | 1%-2% | 0%-1% | 1%-2% | 8%-10% | 0%-1% |
| Ex 1364 | 1%-2% | 0%-1% | 1%-2% | 8%-10% | 1%-2% |
| Ex 1365 | 1%-2% | 0%-1% | 1%-2% | 8%-10% | 2%-4% |
| Ex 1366 | 1%-2% | 0%-1% | 1%-2% | 8%-10% | 4%-6% |
| Ex 1367 | 1%-2% | 0%-1% | 1%-2% | 8%-10% | 6%-8% |
| Ex 1368 | 1%-2% | 0%-1% | 1%-2% | 8%-10% | 8%-10% |
| Ex 1369 | 1%-2% | 0%-1% | 2%-4% | 0%-1% | 0%-1% |
| Ex 1370 | 1%-2% | 0%-1% | 2%-4% | 0%-1% | 1%-2% |
| Ex 1371 | 1%-2% | 0%-1% | 2%-4% | 0%-1% | 2%-4% |
| Ex 1372 | 1%-2% | 0%-1% | 2%-4% | 0%-1% | 4%-6% |
| Ex 1373 | 1%-2% | 0%-1% | 2%-4% | 0%-1% | 6%-8% |
| Ex 1374 | 1%-2% | 0%-1% | 2%-4% | 0%-1% | 8%-10% |
| Ex 1375 | 1%-2% | 0%-1% | 2%-4% | 1%-2% | 0%-1% |
| Ex 1376 | 1%-2% | 0%-1% | 2%-4% | 1%-2% | 1%-2% |
| Ex 1377 | 1%-2% | 0%-1% | 2%-4% | 1%-2% | 2%-4% |
| Ex 1378 | 1%-2% | 0%-1% | 2%-4% | 1%-2% | 4%-6% |
| Ex 1379 | 1%-2% | 0%-1% | 2%-4% | 1%-2% | 6%-8% |
| Ex 1380 | 1%-2% | 0%-1% | 2%-4% | 1%-2% | 8%-10% |
| Ex 1381 | 1%-2% | 0%-1% | 2%-4% | 2%-4% | 0%-1% |
| Ex 1382 | 1%-2% | 0%-1% | 2%-4% | 2%-4% | 1%-2% |
| Ex 1383 | 1%-2% | 0%-1% | 2%-4% | 2%-4% | 2%-4% |
| Ex 1384 | 1%-2% | 0%-1% | 2%-4% | 2%-4% | 4%-6% |
| Ex 1385 | 1%-2% | 0%-1% | 2%-4% | 2%-4% | 6%-8% |
| Ex 1386 | 1%-2% | 0%-1% | 2%-4% | 2%-4% | 8%-10% |
| Ex 1387 | 1%-2% | 0%-1% | 2%-4% | 4%-6% | 0%-1% |
| Ex 1388 | 1%-2% | 0%-1% | 2%-4% | 4%-6% | 1%-2% |
| Ex 1389 | 1%-2% | 0%-1% | 2%-4% | 4%-6% | 2%-4% |
| Ex 1390 | 1%-2% | 0%-1% | 2%-4% | 4%-6% | 4%-6% |
| Ex 1391 | 1%-2% | 0%-1% | 2%-4% | 4%-6% | 6%-8% |
| Ex 1392 | 1%-2% | 0%-1% | 2%-4% | 4%-6% | 8%-10% |
| Ex 1393 | 1%-2% | 0%-1% | 2%-4% | 6%-8% | 0%-1% |
| Ex 1394 | 1%-2% | 0%-1% | 2%-4% | 6%-8% | 1%-2% |
| Ex 1395 | 1%-2% | 0%-1% | 2%-4% | 6%-8% | 2%-4% |
| Ex 1396 | 1%-2% | 0%-1% | 2%-4% | 6%-8% | 4%-6% |
| Ex 1397 | 1%-2% | 0%-1% | 2%-4% | 6%-8% | 6%-8% |
| Ex 1398 | 1%-2% | 0%-1% | 2%-4% | 6%-8% | 8%-10% |
| Ex 1399 | 1%-2% | 0%-1% | 2%-4% | 8%-10% | 0%-1% |
| Ex 1400 | 1%-2% | 0%-1% | 2%-4% | 8%-10% | 1%-2% |
| Ex 1401 | 1%-2% | 0%-1% | 2%-4% | 8%-10% | 2%-4% |
| Ex 1402 | 1%-2% | 0%-1% | 2%-4% | 8%-10% | 4%-6% |
| Ex 1403 | 1%-2% | 0%-1% | 2%-4% | 8%-10% | 6%-8% |
| Ex 1404 | 1%-2% | 0%-1% | 2%-4% | 8%-10% | 8%-10% |
| Ex 1405 | 1%-2% | 0%-1% | 4%-6% | 0%-1% | 0%-1% |
| Ex 1406 | 1%-2% | 0%-1% | 4%-6% | 0%-1% | 1%-2% |
| Ex 1407 | 1%-2% | 0%-1% | 4%-6% | 0%-1% | 2%-4% |
| Ex 1408 | 1%-2% | 0%-1% | 4%-6% | 0%-1% | 4%-6% |
| Ex 1409 | 1%-2% | 0%-1% | 4%-6% | 0%-1% | 6%-8% |
| Ex 1410 | 1%-2% | 0%-1% | 4%-6% | 0%-1% | 8%-10% |
| Ex 1411 | 1%-2% | 0%-1% | 4%-6% | 1%-2% | 0%-1% |
| Ex 1412 | 1%-2% | 0%-1% | 4%-6% | 1%-2% | 1%-2% |
| Ex 1413 | 1%-2% | 0%-1% | 4%-6% | 1%-2% | 2%-4% |
| Ex 1414 | 1%-2% | 0%-1% | 4%-6% | 1%-2% | 4%-6% |
| Ex 1415 | 1%-2% | 0%-1% | 4%-6% | 1%-2% | 6%-8% |
| Ex 1416 | 1%-2% | 0%-1% | 4%-6% | 1%-2% | 8%-10% |
| Ex 1417 | 1%-2% | 0%-1% | 4%-6% | 2%-4% | 0%-1% |
| Ex 1418 | 1%-2% | 0%-1% | 4%-6% | 2%-4% | 1%-2% |
| Ex 1419 | 1%-2% | 0%-1% | 4%-6% | 2%-4% | 2%-4% |
| Ex 1420 | 1%-2% | 0%-1% | 4%-6% | 2%-4% | 4%-6% |
| Ex 1421 | 1%-2% | 0%-1% | 4%-6% | 2%-4% | 6%-8% |
| Ex 1422 | 1%-2% | 0%-1% | 4%-6% | 2%-4% | 8%-10% |
| Ex 1423 | 1%-2% | 0%-1% | 4%-6% | 4%-6% | 0%-1% |
| Ex 1424 | 1%-2% | 0%-1% | 4%-6% | 4%-6% | 1%-2% |
| Ex 1425 | 1%-2% | 0%-1% | 4%-6% | 4%-6% | 2%-4% |
| Ex 1426 | 1%-2% | 0%-1% | 4%-6% | 4%-6% | 4%-6% |
| Ex 1427 | 1%-2% | 0%-1% | 4%-6% | 4%-6% | 6%-8% |
| Ex 1428 | 1%-2% | 0%-1% | 4%-6% | 4%-6% | 8%-10% |
| Ex 1429 | 1%-2% | 0%-1% | 4%-6% | 6%-8% | 0%-1% |
| Ex 1430 | 1%-2% | 0%-1% | 4%-6% | 6%-8% | 1%-2% |
| Ex 1431 | 1%-2% | 0%-1% | 4%-6% | 6%-8% | 2%-4% |
| Ex 1432 | 1%-2% | 0%-1% | 4%-6% | 6%-8% | 4%-6% |
| Ex 1433 | 1%-2% | 0%-1% | 4%-6% | 6%-8% | 6%-8% |
| Ex 1434 | 1%-2% | 0%-1% | 4%-6% | 6%-8% | 8%-10% |
| Ex 1435 | 1%-2% | 0%-1% | 4%-6% | 8%-10% | 0%-1% |
| Ex 1436 | 1%-2% | 0%-1% | 4%-6% | 8%-10% | 1%-2% |
| Ex 1437 | 1%-2% | 0%-1% | 4%-6% | 8%-10% | 2%-4% |
| Ex 1438 | 1%-2% | 0%-1% | 4%-6% | 8%-10% | 4%-6% |
| Ex 1439 | 1%-2% | 0%-1% | 4%-6% | 8%-10% | 6%-8% |
| Ex 1440 | 1%-2% | 0%-1% | 4%-6% | 8%-10% | 8%-10% |
| Ex 1441 | 1%-2% | 0%-1% | 6%-8% | 0%-1% | 0%-1% |
| Ex 1442 | 1%-2% | 0%-1% | 6%-8% | 0%-1% | 1%-2% |
| Ex 1443 | 1%-2% | 0%-1% | 6%-8% | 0%-1% | 2%-4% |
| Ex 1444 | 1%-2% | 0%-1% | 6%-8% | 0%-1% | 4%-6% |
| Ex 1445 | 1%-2% | 0%-1% | 6%-8% | 0%-1% | 6%-8% |
| Ex 1446 | 1%-2% | 0%-1% | 6%-8% | 0%-1% | 8%-10% |
| Ex 1447 | 1%-2% | 0%-1% | 6%-8% | 1%-2% | 0%-1% |
| Ex 1448 | 1%-2% | 0%-1% | 6%-8% | 1%-2% | 1%-2% |
| Ex 1449 | 1%-2% | 0%-1% | 6%-8% | 1%-2% | 2%-4% |
| Ex 1450 | 1%-2% | 0%-1% | 6%-8% | 1%-2% | 4%-6% |
| Ex 1451 | 1%-2% | 0%-1% | 6%-8% | 1%-2% | 6%-8% |
| Ex 1452 | 1%-2% | 0%-1% | 6%-8% | 1%-2% | 8%-10% |
| Ex 1453 | 1%-2% | 0%-1% | 6%-8% | 2%-4% | 0%-1% |
| Ex 1454 | 1%-2% | 0%-1% | 6%-8% | 2%-4% | 1%-2% |
| Ex 1455 | 1%-2% | 0%-1% | 6%-8% | 2%-4% | 2%-4% |
| Ex 1456 | 1%-2% | 0%-1% | 6%-8% | 2%-4% | 4%-6% |
| Ex 1457 | 1%-2% | 0%-1% | 6%-8% | 2%-4% | 6%-8% |
| Ex 1458 | 1%-2% | 0%-1% | 6%-8% | 2%-4% | 8%-10% |
| Ex 1459 | 1%-2% | 0%-1% | 6%-8% | 4%-6% | 0%-1% |
| Ex 1460 | 1%-2% | 0%-1% | 6%-8% | 4%-6% | 1%-2% |
| Ex 1461 | 1%-2% | 0%-1% | 6%-8% | 4%-6% | 2%-4% |
| Ex 1462 | 1%-2% | 0%-1% | 6%-8% | 4%-6% | 4%-6% |
| Ex 1463 | 1%-2% | 0%-1% | 6%-8% | 4%-6% | 6%-8% |
| Ex 1464 | 1%-2% | 0%-1% | 6%-8% | 4%-6% | 8%-10% |
| Ex 1465 | 1%-2% | 0%-1% | 6%-8% | 6%-8% | 0%-1% |
| Ex 1466 | 1%-2% | 0%-1% | 6%-8% | 6%-8% | 1%-2% |
| Ex 1467 | 1%-2% | 0%-1% | 6%-8% | 6%-8% | 2%-4% |
| Ex 1468 | 1%-2% | 0%-1% | 6%-8% | 6%-8% | 4%-6% |
| Ex 1469 | 1%-2% | 0%-1% | 6%-8% | 6%-8% | 6%-8% |
| Ex 1470 | 1%-2% | 0%-1% | 6%-8% | 6%-8% | 8%-10% |
| Ex 1471 | 1%-2% | 0%-1% | 6%-8% | 8%-10% | 0%-1% |
| Ex 1472 | 1%-2% | 0%-1% | 6%-8% | 8%-10% | 1%-2% |
| Ex 1473 | 1%-2% | 0%-1% | 6%-8% | 8%-10% | 2%-4% |
| Ex 1474 | 1%-2% | 0%-1% | 6%-8% | 8%-10% | 4%-6% |
| Ex 1475 | 1%-2% | 0%-1% | 6%-8% | 8%-10% | 6%-8% |
| Ex 1476 | 1%-2% | 0%-1% | 6%-8% | 8%-10% | 8%-10% |
| Ex 1477 | 1%-2% | 0%-1% | 8%-10% | 0%-1% | 0%-1% |
| Ex 1478 | 1%-2% | 0%-1% | 8%-10% | 0%-1% | 1%-2% |
| Ex 1479 | 1%-2% | 0%-1% | 8%-10% | 0%-1% | 2%-4% |
| Ex 1480 | 1%-2% | 0%-1% | 8%-10% | 0%-1% | 4%-6% |
| Ex 1481 | 1%-2% | 0%-1% | 8%-10% | 0%-1% | 6%-8% |
| Ex 1482 | 1%-2% | 0%-1% | 8%-10% | 0%-1% | 8%-10% |
| Ex 1483 | 1%-2% | 0%-1% | 8%-10% | 1%-2% | 0%-1% |
| Ex 1484 | 1%-2% | 0%-1% | 8%-10% | 1%-2% | 1%-2% |
| Ex 1485 | 1%-2% | 0%-1% | 8%-10% | 1%-2% | 2%-4% |
| Ex 1486 | 1%-2% | 0%-1% | 8%-10% | 1%-2% | 4%-6% |
| Ex 1487 | 1%-2% | 0%-1% | 8%-10% | 1%-2% | 6%-8% |
| Ex 1488 | 1%-2% | 0%-1% | 8%-10% | 1%-2% | 8%-10% |
| Ex 1489 | 1%-2% | 0%-1% | 8%-10% | 2%-4% | 0%-1% |
| Ex 1490 | 1%-2% | 0%-1% | 8%-10% | 2%-4% | 1%-2% |
| Ex 1491 | 1%-2% | 0%-1% | 8%-10% | 2%-4% | 2%-4% |
| Ex 1492 | 1%-2% | 0%-1% | 8%-10% | 2%-4% | 4%-6% |
| Ex 1493 | 1%-2% | 0%-1% | 8%-10% | 2%-4% | 6%-8% |
| Ex 1494 | 1%-2% | 0%-1% | 8%-10% | 2%-4% | 8%-10% |
| Ex 1495 | 1%-2% | 0%-1% | 8%-10% | 4%-6% | 0%-1% |
| Ex 1496 | 1%-2% | 0%-1% | 8%-10% | 4%-6% | 1%-2% |
| Ex 1497 | 1%-2% | 0%-1% | 8%-10% | 4%-6% | 2%-4% |
| Ex 1498 | 1%-2% | 0%-1% | 8%-10% | 4%-6% | 4%-6% |
| Ex 1499 | 1%-2% | 0%-1% | 8%-10% | 4%-6% | 6%-8% |
| Ex 1500 | 1%-2% | 0%-1% | 8%-10% | 4%-6% | 8%-10% |
| Ex 1501 | 1%-2% | 0%-1% | 8%-10% | 6%-8% | 0%-1% |
| Ex 1502 | 1%-2% | 0%-1% | 8%-10% | 6%-8% | 1%-2% |
| Ex 1503 | 1%-2% | 0%-1% | 8%-10% | 6%-8% | 2%-4% |
| Ex 1504 | 1%-2% | 0%-1% | 8%-10% | 6%-8% | 4%-6% |
| Ex 1505 | 1%-2% | 0%-1% | 8%-10% | 6%-8% | 6%-8% |
| Ex 1506 | 1%-2% | 0%-1% | 8%-10% | 6%-8% | 8%-10% |
| Ex 1507 | 1%-2% | 0%-1% | 8%-10% | 8%-10% | 0%-1% |
| Ex 1508 | 1%-2% | 0%-1% | 8%-10% | 8%-10% | 1%-2% |
| Ex 1509 | 1%-2% | 0%-1% | 8%-10% | 8%-10% | 2%-4% |
| Ex 1510 | 1%-2% | 0%-1% | 8%-10% | 8%-10% | 4%-6% |
| Ex 1511 | 1%-2% | 0%-1% | 8%-10% | 8%-10% | 6%-8% |
| Ex 1512 | 1%-2% | 0%-1% | 8%-10% | 8%-10% | 8%-10% |
| Ex 1513 | 1%-2% | 1%-2% | 0%-1% | 0%-1% | 0%-1% |
| Ex 1514 | 1%-2% | 1%-2% | 0%-1% | 0%-1% | 1%-2% |
| Ex 1515 | 1%-2% | 1%-2% | 0%-1% | 0%-1% | 2%-4% |
| Ex 1516 | 1%-2% | 1%-2% | 0%-1% | 0%-1% | 4%-6% |
| Ex 1517 | 1%-2% | 1%-2% | 0%-1% | 0%-1% | 6%-8% |
| Ex 1518 | 1%-2% | 1%-2% | 0%-1% | 0%-1% | 8%-10% |
| Ex 1519 | 1%-2% | 1%-2% | 0%-1% | 1%-2% | 0%-1% |
| Ex 1520 | 1%-2% | 1%-2% | 0%-1% | 1%-2% | 1%-2% |
| Ex 1521 | 1%-2% | 1%-2% | 0%-1% | 1%-2% | 2%-4% |
| Ex 1522 | 1%-2% | 1%-2% | 0%-1% | 1%-2% | 4%-6% |
| Ex 1523 | 1%-2% | 1%-2% | 0%-1% | 1%-2% | 6%-8% |
| Ex 1524 | 1%-2% | 1%-2% | 0%-1% | 1%-2% | 8%-10% |
| Ex 1525 | 1%-2% | 1%-2% | 0%-1% | 2%-4% | 0%-1% |
| Ex 1526 | 1%-2% | 1%-2% | 0%-1% | 2%-4% | 1%-2% |
| Ex 1527 | 1%-2% | 1%-2% | 0%-1% | 2%-4% | 2%-4% |
| Ex 1528 | 1%-2% | 1%-2% | 0%-1% | 2%-4% | 4%-6% |
| Ex 1529 | 1%-2% | 1%-2% | 0%-1% | 2%-4% | 6%-8% |
| Ex 1530 | 1%-2% | 1%-2% | 0%-1% | 2%-4% | 8%-10% |
| Ex 1531 | 1%-2% | 1%-2% | 0%-1% | 4%-6% | 0%-1% |
| Ex 1532 | 1%-2% | 1%-2% | 0%-1% | 4%-6% | 1%-2% |
| Ex 1533 | 1%-2% | 1%-2% | 0%-1% | 4%-6% | 2%-4% |
| Ex 1534 | 1%-2% | 1%-2% | 0%-1% | 4%-6% | 4%-6% |
| Ex 1535 | 1%-2% | 1%-2% | 0%-1% | 4%-6% | 6%-8% |
| Ex 1536 | 1%-2% | 1%-2% | 0%-1% | 4%-6% | 8%-10% |
| Ex 1537 | 1%-2% | 1%-2% | 0%-1% | 6%-8% | 0%-1% |
| Ex 1538 | 1%-2% | 1%-2% | 0%-1% | 6%-8% | 1%-2% |
| Ex 1539 | 1%-2% | 1%-2% | 0%-1% | 6%-8% | 2%-4% |
| Ex 1540 | 1%-2% | 1%-2% | 0%-1% | 6%-8% | 4%-6% |
| Ex 1541 | 1%-2% | 1%-2% | 0%-1% | 6%-8% | 6%-8% |
| Ex 1542 | 1%-2% | 1%-2% | 0%-1% | 6%-8% | 8%-10% |
| Ex 1543 | 1%-2% | 1%-2% | 0%-1% | 8%-10% | 0%-1% |
| Ex 1544 | 1%-2% | 1%-2% | 0%-1% | 8%-10% | 1%-2% |
| Ex 1545 | 1%-2% | 1%-2% | 0%-1% | 8%-10% | 2%-4% |
| Ex 1546 | 1%-2% | 1%-2% | 0%-1% | 8%-10% | 4%-6% |
| Ex 1547 | 1%-2% | 1%-2% | 0%-1% | 8%-10% | 6%-8% |
| Ex 1548 | 1%-2% | 1%-2% | 0%-1% | 8%-10% | 8%-10% |
| Ex 1549 | 1%-2% | 1%-2% | 1%-2% | 0%-1% | 0%-1% |
| Ex 1550 | 1%-2% | 1%-2% | 1%-2% | 0%-1% | 1%-2% |
| Ex 1551 | 1%-2% | 1%-2% | 1%-2% | 0%-1% | 2%-4% |
| Ex 1552 | 1%-2% | 1%-2% | 1%-2% | 0%-1% | 4%-6% |
| Ex 1553 | 1%-2% | 1%-2% | 1%-2% | 0%-1% | 6%-8% |
| Ex 1554 | 1%-2% | 1%-2% | 1%-2% | 0%-1% | 8%-10% |
| Ex 1555 | 1%-2% | 1%-2% | 1%-2% | 1%-2% | 0%-1% |
| Ex 1556 | 1%-2% | 1%-2% | 1%-2% | 1%-2% | 1%-2% |
| Ex 1557 | 1%-2% | 1%-2% | 1%-2% | 1%-2% | 2%-4% |
| Ex 1558 | 1%-2% | 1%-2% | 1%-2% | 1%-2% | 4%-6% |
| Ex 1559 | 1%-2% | 1%-2% | 1%-2% | 1%-2% | 6%-8% |
| Ex 1560 | 1%-2% | 1%-2% | 1%-2% | 1%-2% | 8%-10% |
| Ex 1561 | 1%-2% | 1%-2% | 1%-2% | 2%-4% | 0%-1% |
| Ex 1562 | 1%-2% | 1%-2% | 1%-2% | 2%-4% | 1%-2% |
| Ex 1563 | 1%-2% | 1%-2% | 1%-2% | 2%-4% | 2%-4% |
| Ex 1564 | 1%-2% | 1%-2% | 1%-2% | 2%-4% | 4%-6% |
| Ex 1565 | 1%-2% | 1%-2% | 1%-2% | 2%-4% | 6%-8% |
| Ex 1566 | 1%-2% | 1%-2% | 1%-2% | 2%-4% | 8%-10% |
| Ex 1567 | 1%-2% | 1%-2% | 1%-2% | 4%-6% | 0%-1% |
| Ex 1568 | 1%-2% | 1%-2% | 1%-2% | 4%-6% | 1%-2% |
| Ex 1569 | 1%-2% | 1%-2% | 1%-2% | 4%-6% | 2%-4% |
| Ex 1570 | 1%-2% | 1%-2% | 1%-2% | 4%-6% | 4%-6% |
| Ex 1571 | 1%-2% | 1%-2% | 1%-2% | 4%-6% | 6%-8% |
| Ex 1572 | 1%-2% | 1%-2% | 1%-2% | 4%-6% | 8%-10% |
| Ex 1573 | 1%-2% | 1%-2% | 1%-2% | 6%-8% | 0%-1% |
| Ex 1574 | 1%-2% | 1%-2% | 1%-2% | 6%-8% | 1%-2% |
| Ex 1575 | 1%-2% | 1%-2% | 1%-2% | 6%-8% | 2%-4% |
| Ex 1576 | 1%-2% | 1%-2% | 1%-2% | 6%-8% | 4%-6% |
| Ex 1577 | 1%-2% | 1%-2% | 1%-2% | 6%-8% | 6%-8% |
| Ex 1578 | 1%-2% | 1%-2% | 1%-2% | 6%-8% | 8%-10% |
| Ex 1579 | 1%-2% | 1%-2% | 1%-2% | 8%-10% | 0%-1% |
| Ex 1580 | 1%-2% | 1%-2% | 1%-2% | 8%-10% | 1%-2% |
| Ex 1581 | 1%-2% | 1%-2% | 1%-2% | 8%-10% | 2%-4% |
| Ex 1582 | 1%-2% | 1%-2% | 1%-2% | 8%-10% | 4%-6% |
| Ex 1583 | 1%-2% | 1%-2% | 1%-2% | 8%-10% | 6%-8% |
| Ex 1584 | 1%-2% | 1%-2% | 1%-2% | 8%-10% | 8%-10% |
| Ex 1585 | 1%-2% | 1%-2% | 2%-4% | 0%-1% | 0%-1% |
| Ex 1586 | 1%-2% | 1%-2% | 2%-4% | 0%-1% | 1%-2% |
| Ex 1587 | 1%-2% | 1%-2% | 2%-4% | 0%-1% | 2%-4% |
| Ex 1588 | 1%-2% | 1%-2% | 2%-4% | 0%-1% | 4%-6% |
| Ex 1589 | 1%-2% | 1%-2% | 2%-4% | 0%-1% | 6%-8% |
| Ex 1590 | 1%-2% | 1%-2% | 2%-4% | 0%-1% | 8%-10% |
| Ex 1591 | 1%-2% | 1%-2% | 2%-4% | 1%-2% | 0%-1% |
| Ex 1592 | 1%-2% | 1%-2% | 2%-4% | 1%-2% | 1%-2% |
| Ex 1593 | 1%-2% | 1%-2% | 2%-4% | 1%-2% | 2%-4% |
| Ex 1594 | 1%-2% | 1%-2% | 2%-4% | 1%-2% | 4%-6% |
| Ex 1595 | 1%-2% | 1%-2% | 2%-4% | 1%-2% | 6%-8% |
| Ex 1596 | 1%-2% | 1%-2% | 2%-4% | 1%-2% | 8%-10% |
| Ex 1597 | 1%-2% | 1%-2% | 2%-4% | 2%-4% | 0%-1% |
| Ex 1598 | 1%-2% | 1%-2% | 2%-4% | 2%-4% | 1%-2% |
| Ex 1599 | 1%-2% | 1%-2% | 2%-4% | 2%-4% | 2%-4% |
| Ex 1600 | 1%-2% | 1%-2% | 2%-4% | 2%-4% | 4%-6% |
| Ex 1601 | 1%-2% | 1%-2% | 2%-4% | 2%-4% | 6%-8% |
| Ex 1602 | 1%-2% | 1%-2% | 2%-4% | 2%-4% | 8%-10% |
| Ex 1603 | 1%-2% | 1%-2% | 2%-4% | 4%-6% | 0%-1% |
| Ex 1604 | 1%-2% | 1%-2% | 2%-4% | 4%-6% | 1%-2% |
| Ex 1605 | 1%-2% | 1%-2% | 2%-4% | 4%-6% | 2%-4% |
| Ex 1606 | 1%-2% | 1%-2% | 2%-4% | 4%-6% | 4%-6% |
| Ex 1607 | 1%-2% | 1%-2% | 2%-4% | 4%-6% | 6%-8% |
| Ex 1608 | 1%-2% | 1%-2% | 2%-4% | 4%-6% | 8%-10% |
| Ex 1609 | 1%-2% | 1%-2% | 2%-4% | 6%-8% | 0%-1% |
| Ex 1610 | 1%-2% | 1%-2% | 2%-4% | 6%-8% | 1%-2% |
| Ex 1611 | 1%-2% | 1%-2% | 2%-4% | 6%-8% | 2%-4% |
| Ex 1612 | 1%-2% | 1%-2% | 2%-4% | 6%-8% | 4%-6% |
| Ex 1613 | 1%-2% | 1%-2% | 2%-4% | 6%-8% | 6%-8% |
| Ex 1614 | 1%-2% | 1%-2% | 2%-4% | 6%-8% | 8%-10% |
| Ex 1615 | 1%-2% | 1%-2% | 2%-4% | 8%-10% | 0%-1% |
| Ex 1616 | 1%-2% | 1%-2% | 2%-4% | 8%-10% | 1%-2% |
| Ex 1617 | 1%-2% | 1%-2% | 2%-4% | 8%-10% | 2%-4% |
| Ex 1618 | 1%-2% | 1%-2% | 2%-4% | 8%-10% | 4%-6% |
| Ex 1619 | 1%-2% | 1%-2% | 2%-4% | 8%-10% | 6%-8% |
| Ex 1620 | 1%-2% | 1%-2% | 2%-4% | 8%-10% | 8%-10% |
| Ex 1621 | 1%-2% | 1%-2% | 4%-6% | 0%-1% | 0%-1% |
| Ex 1622 | 1%-2% | 1%-2% | 4%-6% | 0%-1% | 1%-2% |
| Ex 1623 | 1%-2% | 1%-2% | 4%-6% | 0%-1% | 2%-4% |
| Ex 1624 | 1%-2% | 1%-2% | 4%-6% | 0%-1% | 4%-6% |
| Ex 1625 | 1%-2% | 1%-2% | 4%-6% | 0%-1% | 6%-8% |
| Ex 1626 | 1%-2% | 1%-2% | 4%-6% | 0%-1% | 8%-10% |
| Ex 1627 | 1%-2% | 1%-2% | 4%-6% | 1%-2% | 0%-1% |
| Ex 1628 | 1%-2% | 1%-2% | 4%-6% | 1%-2% | 1%-2% |
| Ex 1629 | 1%-2% | 1%-2% | 4%-6% | 1%-2% | 2%-4% |
| Ex 1630 | 1%-2% | 1%-2% | 4%-6% | 1%-2% | 4%-6% |
| Ex 1631 | 1%-2% | 1%-2% | 4%-6% | 1%-2% | 6%-8% |
| Ex 1632 | 1%-2% | 1%-2% | 4%-6% | 1%-2% | 8%-10% |
| Ex 1633 | 1%-2% | 1%-2% | 4%-6% | 2%-4% | 0%-1% |
| Ex 1634 | 1%-2% | 1%-2% | 4%-6% | 2%-4% | 1%-2% |
| Ex 1635 | 1%-2% | 1%-2% | 4%-6% | 2%-4% | 2%-4% |
| Ex 1636 | 1%-2% | 1%-2% | 4%-6% | 2%-4% | 4%-6% |
| Ex 1637 | 1%-2% | 1%-2% | 4%-6% | 2%-4% | 6%-8% |
| Ex 1638 | 1%-2% | 1%-2% | 4%-6% | 2%-4% | 8%-10% |
| Ex 1639 | 1%-2% | 1%-2% | 4%-6% | 4%-6% | 0%-1% |
| Ex 1640 | 1%-2% | 1%-2% | 4%-6% | 4%-6% | 1%-2% |
| Ex 1641 | 1%-2% | 1%-2% | 4%-6% | 4%-6% | 2%-4% |
| Ex 1642 | 1%-2% | 1%-2% | 4%-6% | 4%-6% | 4%-6% |
| Ex 1643 | 1%-2% | 1%-2% | 4%-6% | 4%-6% | 6%-8% |
| Ex 1644 | 1%-2% | 1%-2% | 4%-6% | 4%-6% | 8%-10% |
| Ex 1645 | 1%-2% | 1%-2% | 4%-6% | 6%-8% | 0%-1% |
| Ex 1646 | 1%-2% | 1%-2% | 4%-6% | 6%-8% | 1%-2% |
| Ex 1647 | 1%-2% | 1%-2% | 4%-6% | 6%-8% | 2%-4% |
| Ex 1648 | 1%-2% | 1%-2% | 4%-6% | 6%-8% | 4%-6% |
| Ex 1649 | 1%-2% | 1%-2% | 4%-6% | 6%-8% | 6%-8% |
| Ex 1650 | 1%-2% | 1%-2% | 4%-6% | 6%-8% | 8%-10% |
| Ex 1651 | 1%-2% | 1%-2% | 4%-6% | 8%-10% | 0%-1% |
| Ex 1652 | 1%-2% | 1%-2% | 4%-6% | 8%-10% | 1%-2% |
| Ex 1653 | 1%-2% | 1%-2% | 4%-6% | 8%-10% | 2%-4% |
| Ex 1654 | 1%-2% | 1%-2% | 4%-6% | 8%-10% | 4%-6% |
| Ex 1655 | 1%-2% | 1%-2% | 4%-6% | 8%-10% | 6%-8% |
| Ex 1656 | 1%-2% | 1%-2% | 4%-6% | 8%-10% | 8%-10% |
| Ex 1657 | 1%-2% | 1%-2% | 6%-8% | 0%-1% | 0%-1% |
| Ex 1658 | 1%-2% | 1%-2% | 6%-8% | 0%-1% | 1%-2% |
| Ex 1659 | 1%-2% | 1%-2% | 6%-8% | 0%-1% | 2%-4% |
| Ex 1660 | 1%-2% | 1%-2% | 6%-8% | 0%-1% | 4%-6% |
| Ex 1661 | 1%-2% | 1%-2% | 6%-8% | 0%-1% | 6%-8% |
| Ex 1662 | 1%-2% | 1%-2% | 6%-8% | 0%-1% | 8%-10% |
| Ex 1663 | 1%-2% | 1%-2% | 6%-8% | 1%-2% | 0%-1% |
| Ex 1664 | 1%-2% | 1%-2% | 6%-8% | 1%-2% | 1%-2% |
| Ex 1665 | 1%-2% | 1%-2% | 6%-8% | 1%-2% | 2%-4% |
| Ex 1666 | 1%-2% | 1%-2% | 6%-8% | 1%-2% | 4%-6% |
| Ex 1667 | 1%-2% | 1%-2% | 6%-8% | 1%-2% | 6%-8% |
| Ex 1668 | 1%-2% | 1%-2% | 6%-8% | 1%-2% | 8%-10% |
| Ex 1669 | 1%-2% | 1%-2% | 6%-8% | 2%-4% | 0%-1% |
| Ex 1670 | 1%-2% | 1%-2% | 6%-8% | 2%-4% | 1%-2% |
| Ex 1671 | 1%-2% | 1%-2% | 6%-8% | 2%-4% | 2%-4% |
| Ex 1672 | 1%-2% | 1%-2% | 6%-8% | 2%-4% | 4%-6% |
| Ex 1673 | 1%-2% | 1%-2% | 6%-8% | 2%-4% | 6%-8% |
| Ex 1674 | 1%-2% | 1%-2% | 6%-8% | 2%-4% | 8%-10% |
| Ex 1675 | 1%-2% | 1%-2% | 6%-8% | 4%-6% | 0%-1% |
| Ex 1676 | 1%-2% | 1%-2% | 6%-8% | 4%-6% | 1%-2% |
| Ex 1677 | 1%-2% | 1%-2% | 6%-8% | 4%-6% | 2%-4% |
| Ex 1678 | 1%-2% | 1%-2% | 6%-8% | 4%-6% | 4%-6% |
| Ex 1679 | 1%-2% | 1%-2% | 6%-8% | 4%-6% | 6%-8% |
| Ex 1680 | 1%-2% | 1%-2% | 6%-8% | 4%-6% | 8%-10% |
| Ex 1681 | 1%-2% | 1%-2% | 6%-8% | 6%-8% | 0%-1% |
| Ex 1682 | 1%-2% | 1%-2% | 6%-8% | 6%-8% | 1%-2% |
| Ex 1683 | 1%-2% | 1%-2% | 6%-8% | 6%-8% | 2%-4% |
| Ex 1684 | 1%-2% | 1%-2% | 6%-8% | 6%-8% | 4%-6% |
| Ex 1685 | 1%-2% | 1%-2% | 6%-8% | 6%-8% | 6%-8% |
| Ex 1686 | 1%-2% | 1%-2% | 6%-8% | 6%-8% | 8%-10% |
| Ex 1687 | 1%-2% | 1%-2% | 6%-8% | 8%-10% | 0%-1% |
| Ex 1688 | 1%-2% | 1%-2% | 6%-8% | 8%-10% | 1%-2% |
| Ex 1689 | 1%-2% | 1%-2% | 6%-8% | 8%-10% | 2%-4% |
| Ex 1690 | 1%-2% | 1%-2% | 6%-8% | 8%-10% | 4%-6% |
| Ex 1691 | 1%-2% | 1%-2% | 6%-8% | 8%-10% | 6%-8% |
| Ex 1692 | 1%-2% | 1%-2% | 6%-8% | 8%-10% | 8%-10% |
| Ex 1693 | 1%-2% | 1%-2% | 8%-10% | 0%-1% | 0%-1% |
| Ex 1694 | 1%-2% | 1%-2% | 8%-10% | 0%-1% | 1%-2% |
| Ex 1695 | 1%-2% | 1%-2% | 8%-10% | 0%-1% | 2%-4% |
| Ex 1696 | 1%-2% | 1%-2% | 8%-10% | 0%-1% | 4%-6% |
| Ex 1697 | 1%-2% | 1%-2% | 8%-10% | 0%-1% | 6%-8% |
| Ex 1698 | 1%-2% | 1%-2% | 8%-10% | 0%-1% | 8%-10% |
| Ex 1699 | 1%-2% | 1%-2% | 8%-10% | 1%-2% | 0%-1% |
| Ex 1700 | 1%-2% | 1%-2% | 8%-10% | 1%-2% | 1%-2% |
| Ex 1701 | 1%-2% | 1%-2% | 8%-10% | 1%-2% | 2%-4% |
| Ex 1702 | 1%-2% | 1%-2% | 8%-10% | 1%-2% | 4%-6% |
| Ex 1703 | 1%-2% | 1%-2% | 8%-10% | 1%-2% | 6%-8% |
| Ex 1704 | 1%-2% | 1%-2% | 8%-10% | 1%-2% | 8%-10% |
| Ex 1705 | 1%-2% | 1%-2% | 8%-10% | 2%-4% | 0%-1% |
| Ex 1706 | 1%-2% | 1%-2% | 8%-10% | 2%-4% | 1%-2% |
| Ex 1707 | 1%-2% | 1%-2% | 8%-10% | 2%-4% | 2%-4% |
| Ex 1708 | 1%-2% | 1%-2% | 8%-10% | 2%-4% | 4%-6% |
| Ex 1709 | 1%-2% | 1%-2% | 8%-10% | 2%-4% | 6%-8% |
| Ex 1710 | 1%-2% | 1%-2% | 8%-10% | 2%-4% | 8%-10% |
| Ex 1711 | 1%-2% | 1%-2% | 8%-10% | 4%-6% | 0%-1% |
| Ex 1712 | 1%-2% | 1%-2% | 8%-10% | 4%-6% | 1%-2% |
| Ex 1713 | 1%-2% | 1%-2% | 8%-10% | 4%-6% | 2%-4% |
| Ex 1714 | 1%-2% | 1%-2% | 8%-10% | 4%-6% | 4%-6% |
| Ex 1715 | 1%-2% | 1%-2% | 8%-10% | 4%-6% | 6%-8% |
| Ex 1716 | 1%-2% | 1%-2% | 8%-10% | 4%-6% | 8%-10% |
| Ex 1717 | 1%-2% | 1%-2% | 8%-10% | 6%-8% | 0%-1% |
| Ex 1718 | 1%-2% | 1%-2% | 8%-10% | 6%-8% | 1%-2% |
| Ex 1719 | 1%-2% | 1%-2% | 8%-10% | 6%-8% | 2%-4% |
| Ex 1720 | 1%-2% | 1%-2% | 8%-10% | 6%-8% | 4%-6% |
| Ex 1721 | 1%-2% | 1%-2% | 8%-10% | 6%-8% | 6%-8% |
| Ex 1722 | 1%-2% | 1%-2% | 8%-10% | 6%-8% | 8%-10% |
| Ex 1723 | 1%-2% | 1%-2% | 8%-10% | 8%-10% | 0%-1% |
| Ex 1724 | 1%-2% | 1%-2% | 8%-10% | 8%-10% | 1%-2% |
| Ex 1725 | 1%-2% | 1%-2% | 8%-10% | 8%-10% | 2%-4% |
| Ex 1726 | 1%-2% | 1%-2% | 8%-10% | 8%-10% | 4%-6% |
| Ex 1727 | 1%-2% | 1%-2% | 8%-10% | 8%-10% | 6%-8% |
| Ex 1728 | 1%-2% | 1%-2% | 8%-10% | 8%-10% | 8%-10% |
| Ex 1729 | 1%-2% | 2%-4% | 0%-1% | 0%-1% | 0%-1% |
| Ex 1730 | 1%-2% | 2%-4% | 0%-1% | 0%-1% | 1%-2% |
| Ex 1731 | 1%-2% | 2%-4% | 0%-1% | 0%-1% | 2%-4% |
| Ex 1732 | 1%-2% | 2%-4% | 0%-1% | 0%-1% | 4%-6% |
| Ex 1733 | 1%-2% | 2%-4% | 0%-1% | 0%-1% | 6%-8% |
| Ex 1734 | 1%-2% | 2%-4% | 0%-1% | 0%-1% | 8%-10% |
| Ex 1735 | 1%-2% | 2%-4% | 0%-1% | 1%-2% | 0%-1% |
| Ex 1736 | 1%-2% | 2%-4% | 0%-1% | 1%-2% | 1%-2% |
| Ex 1737 | 1%-2% | 2%-4% | 0%-1% | 1%-2% | 2%-4% |
| Ex 1738 | 1%-2% | 2%-4% | 0%-1% | 1%-2% | 4%-6% |
| Ex 1739 | 1%-2% | 2%-4% | 0%-1% | 1%-2% | 6%-8% |
| Ex 1740 | 1%-2% | 2%-4% | 0%-1% | 1%-2% | 8%-10% |
| Ex 1741 | 1%-2% | 2%-4% | 0%-1% | 2%-4% | 0%-1% |
| Ex 1742 | 1%-2% | 2%-4% | 0%-1% | 2%-4% | 1%-2% |
| Ex 1743 | 1%-2% | 2%-4% | 0%-1% | 2%-4% | 2%-4% |
| Ex 1744 | 1%-2% | 2%-4% | 0%-1% | 2%-4% | 4%-6% |
| Ex 1745 | 1%-2% | 2%-4% | 0%-1% | 2%-4% | 6%-8% |
| Ex 1746 | 1%-2% | 2%-4% | 0%-1% | 2%-4% | 8%-10% |
| Ex 1747 | 1%-2% | 2%-4% | 0%-1% | 4%-6% | 0%-1% |
| Ex 1748 | 1%-2% | 2%-4% | 0%-1% | 4%-6% | 1%-2% |
| Ex 1749 | 1%-2% | 2%-4% | 0%-1% | 4%-6% | 2%-4% |
| Ex 1750 | 1%-2% | 2%-4% | 0%-1% | 4%-6% | 4%-6% |
| Ex 1751 | 1%-2% | 2%-4% | 0%-1% | 4%-6% | 6%-8% |
| Ex 1752 | 1%-2% | 2%-4% | 0%-1% | 4%-6% | 8%-10% |
| Ex 1753 | 1%-2% | 2%-4% | 0%-1% | 6%-8% | 0%-1% |
| Ex 1754 | 1%-2% | 2%-4% | 0%-1% | 6%-8% | 1%-2% |
| Ex 1755 | 1%-2% | 2%-4% | 0%-1% | 6%-8% | 2%-4% |
| Ex 1756 | 1%-2% | 2%-4% | 0%-1% | 6%-8% | 4%-6% |
| Ex 1757 | 1%-2% | 2%-4% | 0%-1% | 6%-8% | 6%-8% |
| Ex 1758 | 1%-2% | 2%-4% | 0%-1% | 6%-8% | 8%-10% |
| Ex 1759 | 1%-2% | 2%-4% | 0%-1% | 8%-10% | 0%-1% |
| Ex 1760 | 1%-2% | 2%-4% | 0%-1% | 8%-10% | 1%-2% |
| Ex 1761 | 1%-2% | 2%-4% | 0%-1% | 8%-10% | 2%-4% |
| Ex 1762 | 1%-2% | 2%-4% | 0%-1% | 8%-10% | 4%-6% |
| Ex 1763 | 1%-2% | 2%-4% | 0%-1% | 8%-10% | 6%-8% |
| Ex 1764 | 1%-2% | 2%-4% | 0%-1% | 8%-10% | 8%-10% |
| Ex 1765 | 1%-2% | 2%-4% | 1%-2% | 0%-1% | 0%-1% |
| Ex 1766 | 1%-2% | 2%-4% | 1%-2% | 0%-1% | 1%-2% |
| Ex 1767 | 1%-2% | 2%-4% | 1%-2% | 0%-1% | 2%-4% |
| Ex 1768 | 1%-2% | 2%-4% | 1%-2% | 0%-1% | 4%-6% |
| Ex 1769 | 1%-2% | 2%-4% | 1%-2% | 0%-1% | 6%-8% |
| Ex 1770 | 1%-2% | 2%-4% | 1%-2% | 0%-1% | 8%-10% |
| Ex 1771 | 1%-2% | 2%-4% | 1%-2% | 1%-2% | 0%-1% |
| Ex 1772 | 1%-2% | 2%-4% | 1%-2% | 1%-2% | 1%-2% |
| Ex 1773 | 1%-2% | 2%-4% | 1%-2% | 1%-2% | 2%-4% |
| Ex 1774 | 1%-2% | 2%-4% | 1%-2% | 1%-2% | 4%-6% |
| Ex 1775 | 1%-2% | 2%-4% | 1%-2% | 1%-2% | 6%-8% |
| Ex 1776 | 1%-2% | 2%-4% | 1%-2% | 1%-2% | 8%-10% |
| Ex 1777 | 1%-2% | 2%-4% | 1%-2% | 2%-4% | 0%-1% |
| Ex 1778 | 1%-2% | 2%-4% | 1%-2% | 2%-4% | 1%-2% |
| Ex 1779 | 1%-2% | 2%-4% | 1%-2% | 2%-4% | 2%-4% |
| Ex 1780 | 1%-2% | 2%-4% | 1%-2% | 2%-4% | 4%-6% |
| Ex 1781 | 1%-2% | 2%-4% | 1%-2% | 2%-4% | 6%-8% |
| Ex 1782 | 1%-2% | 2%-4% | 1%-2% | 2%-4% | 8%-10% |
| Ex 1783 | 1%-2% | 2%-4% | 1%-2% | 4%-6% | 0%-1% |
| Ex 1784 | 1%-2% | 2%-4% | 1%-2% | 4%-6% | 1%-2% |
| Ex 1785 | 1%-2% | 2%-4% | 1%-2% | 4%-6% | 2%-4% |
| Ex 1786 | 1%-2% | 2%-4% | 1%-2% | 4%-6% | 4%-6% |
| Ex 1787 | 1%-2% | 2%-4% | 1%-2% | 4%-6% | 6%-8% |
| Ex 1788 | 1%-2% | 2%-4% | 1%-2% | 4%-6% | 8%-10% |
| Ex 1789 | 1%-2% | 2%-4% | 1%-2% | 6%-8% | 0%-1% |
| Ex 1790 | 1%-2% | 2%-4% | 1%-2% | 6%-8% | 1%-2% |
| Ex 1791 | 1%-2% | 2%-4% | 1%-2% | 6%-8% | 2%-4% |
| Ex 1792 | 1%-2% | 2%-4% | 1%-2% | 6%-8% | 4%-6% |
| Ex 1793 | 1%-2% | 2%-4% | 1%-2% | 6%-8% | 6%-8% |
| Ex 1794 | 1%-2% | 2%-4% | 1%-2% | 6%-8% | 8%-10% |
| Ex 1795 | 1%-2% | 2%-4% | 1%-2% | 8%-10% | 0%-1% |
| Ex 1796 | 1%-2% | 2%-4% | 1%-2% | 8%-10% | 1%-2% |
| Ex 1797 | 1%-2% | 2%-4% | 1%-2% | 8%-10% | 2%-4% |
| Ex 1798 | 1%-2% | 2%-4% | 1%-2% | 8%-10% | 4%-6% |
| Ex 1799 | 1%-2% | 2%-4% | 1%-2% | 8%-10% | 6%-8% |
| Ex 1800 | 1%-2% | 2%-4% | 1%-2% | 8%-10% | 8%-10% |
| Ex 1801 | 1%-2% | 2%-4% | 2%-4% | 0%-1% | 0%-1% |
| Ex 1802 | 1%-2% | 2%-4% | 2%-4% | 0%-1% | 1%-2% |
| Ex 1803 | 1%-2% | 2%-4% | 2%-4% | 0%-1% | 2%-4% |
| Ex 1804 | 1%-2% | 2%-4% | 2%-4% | 0%-1% | 4%-6% |
| Ex 1805 | 1%-2% | 2%-4% | 2%-4% | 0%-1% | 6%-8% |
| Ex 1806 | 1%-2% | 2%-4% | 2%-4% | 0%-1% | 8%-10% |
| Ex 1807 | 1%-2% | 2%-4% | 2%-4% | 1%-2% | 0%-1% |
| Ex 1808 | 1%-2% | 2%-4% | 2%-4% | 1%-2% | 1%-2% |
| Ex 1809 | 1%-2% | 2%-4% | 2%-4% | 1%-2% | 2%-4% |
| Ex 1810 | 1%-2% | 2%-4% | 2%-4% | 1%-2% | 4%-6% |
| Ex 1811 | 1%-2% | 2%-4% | 2%-4% | 1%-2% | 6%-8% |
| Ex 1812 | 1%-2% | 2%-4% | 2%-4% | 1%-2% | 8%-10% |
| Ex 1813 | 1%-2% | 2%-4% | 2%-4% | 2%-4% | 0%-1% |
| Ex 1814 | 1%-2% | 2%-4% | 2%-4% | 2%-4% | 1%-2% |
| Ex 1815 | 1%-2% | 2%-4% | 2%-4% | 2%-4% | 2%-4% |
| Ex 1816 | 1%-2% | 2%-4% | 2%-4% | 2%-4% | 4%-6% |
| Ex 1817 | 1%-2% | 2%-4% | 2%-4% | 2%-4% | 6%-8% |
| Ex 1818 | 1%-2% | 2%-4% | 2%-4% | 2%-4% | 8%-10% |
| Ex 1819 | 1%-2% | 2%-4% | 2%-4% | 4%-6% | 0%-1% |
| Ex 1820 | 1%-2% | 2%-4% | 2%-4% | 4%-6% | 1%-2% |
| Ex 1821 | 1%-2% | 2%-4% | 2%-4% | 4%-6% | 2%-4% |
| Ex 1822 | 1%-2% | 2%-4% | 2%-4% | 4%-6% | 4%-6% |
| Ex 1823 | 1%-2% | 2%-4% | 2%-4% | 4%-6% | 6%-8% |
| Ex 1824 | 1%-2% | 2%-4% | 2%-4% | 4%-6% | 8%-10% |
| Ex 1825 | 1%-2% | 2%-4% | 2%-4% | 6%-8% | 0%-1% |
| Ex 1826 | 1%-2% | 2%-4% | 2%-4% | 6%-8% | 1%-2% |
| Ex 1827 | 1%-2% | 2%-4% | 2%-4% | 6%-8% | 2%-4% |
| Ex 1828 | 1%-2% | 2%-4% | 2%-4% | 6%-8% | 4%-6% |
| Ex 1829 | 1%-2% | 2%-4% | 2%-4% | 6%-8% | 6%-8% |
| Ex 1830 | 1%-2% | 2%-4% | 2%-4% | 6%-8% | 8%-10% |
| Ex 1831 | 1%-2% | 2%-4% | 2%-4% | 8%-10% | 0%-1% |
| Ex 1832 | 1%-2% | 2%-4% | 2%-4% | 8%-10% | 1%-2% |
| Ex 1833 | 1%-2% | 2%-4% | 2%-4% | 8%-10% | 2%-4% |
| Ex 1834 | 1%-2% | 2%-4% | 2%-4% | 8%-10% | 4%-6% |
| Ex 1835 | 1%-2% | 2%-4% | 2%-4% | 8%-10% | 6%-8% |
| Ex 1836 | 1%-2% | 2%-4% | 2%-4% | 8%-10% | 8%-10% |
| Ex 1837 | 1%-2% | 2%-4% | 4%-6% | 0%-1% | 0%-1% |
| Ex 1838 | 1%-2% | 2%-4% | 4%-6% | 0%-1% | 1%-2% |
| Ex 1839 | 1%-2% | 2%-4% | 4%-6% | 0%-1% | 2%-4% |
| Ex 1840 | 1%-2% | 2%-4% | 4%-6% | 0%-1% | 4%-6% |
| Ex 1841 | 1%-2% | 2%-4% | 4%-6% | 0%-1% | 6%-8% |
| Ex 1842 | 1%-2% | 2%-4% | 4%-6% | 0%-1% | 8%-10% |
| Ex 1843 | 1%-2% | 2%-4% | 4%-6% | 1%-2% | 0%-1% |
| Ex 1844 | 1%-2% | 2%-4% | 4%-6% | 1%-2% | 1%-2% |
| Ex 1845 | 1%-2% | 2%-4% | 4%-6% | 1%-2% | 2%-4% |
| Ex 1846 | 1%-2% | 2%-4% | 4%-6% | 1%-2% | 4%-6% |
| Ex 1847 | 1%-2% | 2%-4% | 4%-6% | 1%-2% | 6%-8% |
| Ex 1848 | 1%-2% | 2%-4% | 4%-6% | 1%-2% | 8%-10% |
| Ex 1849 | 1%-2% | 2%-4% | 4%-6% | 2%-4% | 0%-1% |
| Ex 1850 | 1%-2% | 2%-4% | 4%-6% | 2%-4% | 1%-2% |
| Ex 1851 | 1%-2% | 2%-4% | 4%-6% | 2%-4% | 2%-4% |
| Ex 1852 | 1%-2% | 2%-4% | 4%-6% | 2%-4% | 4%-6% |
| Ex 1853 | 1%-2% | 2%-4% | 4%-6% | 2%-4% | 6%-8% |
| Ex 1854 | 1%-2% | 2%-4% | 4%-6% | 2%-4% | 8%-10% |
| Ex 1855 | 1%-2% | 2%-4% | 4%-6% | 4%-6% | 0%-1% |
| Ex 1856 | 1%-2% | 2%-4% | 4%-6% | 4%-6% | 1%-2% |
| Ex 1857 | 1%-2% | 2%-4% | 4%-6% | 4%-6% | 2%-4% |
| Ex 1858 | 1%-2% | 2%-4% | 4%-6% | 4%-6% | 4%-6% |
| Ex 1859 | 1%-2% | 2%-4% | 4%-6% | 4%-6% | 6%-8% |
| Ex 1860 | 1%-2% | 2%-4% | 4%-6% | 4%-6% | 8%-10% |
| Ex 1861 | 1%-2% | 2%-4% | 4%-6% | 6%-8% | 0%-1% |
| Ex 1862 | 1%-2% | 2%-4% | 4%-6% | 6%-8% | 1%-2% |
| Ex 1863 | 1%-2% | 2%-4% | 4%-6% | 6%-8% | 2%-4% |
| Ex 1864 | 1%-2% | 2%-4% | 4%-6% | 6%-8% | 4%-6% |
| Ex 1865 | 1%-2% | 2%-4% | 4%-6% | 6%-8% | 6%-8% |
| Ex 1866 | 1%-2% | 2%-4% | 4%-6% | 6%-8% | 8%-10% |
| Ex 1867 | 1%-2% | 2%-4% | 4%-6% | 8%-10% | 0%-1% |
| Ex 1868 | 1%-2% | 2%-4% | 4%-6% | 8%-10% | 1%-2% |
| Ex 1869 | 1%-2% | 2%-4% | 4%-6% | 8%-10% | 2%-4% |
| Ex 1870 | 1%-2% | 2%-4% | 4%-6% | 8%-10% | 4%-6% |
| Ex 1871 | 1%-2% | 2%-4% | 4%-6% | 8%-10% | 6%-8% |
| Ex 1872 | 1%-2% | 2%-4% | 4%-6% | 8%-10% | 8%-10% |
| Ex 1873 | 1%-2% | 2%-4% | 6%-8% | 0%-1% | 0%-1% |
| Ex 1874 | 1%-2% | 2%-4% | 6%-8% | 0%-1% | 1%-2% |
| Ex 1875 | 1%-2% | 2%-4% | 6%-8% | 0%-1% | 2%-4% |
| Ex 1876 | 1%-2% | 2%-4% | 6%-8% | 0%-1% | 4%-6% |
| Ex 1877 | 1%-2% | 2%-4% | 6%-8% | 0%-1% | 6%-8% |
| Ex 1878 | 1%-2% | 2%-4% | 6%-8% | 0%-1% | 8%-10% |
| Ex 1879 | 1%-2% | 2%-4% | 6%-8% | 1%-2% | 0%-1% |
| Ex 1880 | 1%-2% | 2%-4% | 6%-8% | 1%-2% | 1%-2% |
| Ex 1881 | 1%-2% | 2%-4% | 6%-8% | 1%-2% | 2%-4% |
| Ex 1882 | 1%-2% | 2%-4% | 6%-8% | 1%-2% | 4%-6% |
| Ex 1883 | 1%-2% | 2%-4% | 6%-8% | 1%-2% | 6%-8% |
| Ex 1884 | 1%-2% | 2%-4% | 6%-8% | 1%-2% | 8%-10% |
| Ex 1885 | 1%-2% | 2%-4% | 6%-8% | 2%-4% | 0%-1% |
| Ex 1886 | 1%-2% | 2%-4% | 6%-8% | 2%-4% | 1%-2% |
| Ex 1887 | 1%-2% | 2%-4% | 6%-8% | 2%-4% | 2%-4% |
| Ex 1888 | 1%-2% | 2%-4% | 6%-8% | 2%-4% | 4%-6% |
| Ex 1889 | 1%-2% | 2%-4% | 6%-8% | 2%-4% | 6%-8% |
| Ex 1890 | 1%-2% | 2%-4% | 6%-8% | 2%-4% | 8%-10% |
| Ex 1891 | 1%-2% | 2%-4% | 6%-8% | 4%-6% | 0%-1% |
| Ex 1892 | 1%-2% | 2%-4% | 6%-8% | 4%-6% | 1%-2% |
| Ex 1893 | 1%-2% | 2%-4% | 6%-8% | 4%-6% | 2%-4% |
| Ex 1894 | 1%-2% | 2%-4% | 6%-8% | 4%-6% | 4%-6% |
| Ex 1895 | 1%-2% | 2%-4% | 6%-8% | 4%-6% | 6%-8% |
| Ex 1896 | 1%-2% | 2%-4% | 6%-8% | 4%-6% | 8%-10% |
| Ex 1897 | 1%-2% | 2%-4% | 6%-8% | 6%-8% | 0%-1% |
| Ex 1898 | 1%-2% | 2%-4% | 6%-8% | 6%-8% | 1%-2% |
| Ex 1899 | 1%-2% | 2%-4% | 6%-8% | 6%-8% | 2%-4% |
| Ex 1900 | 1%-2% | 2%-4% | 6%-8% | 6%-8% | 4%-6% |
| Ex 1901 | 1%-2% | 2%-4% | 6%-8% | 6%-8% | 6%-8% |
| Ex 1902 | 1%-2% | 2%-4% | 6%-8% | 6%-8% | 8%-10% |
| Ex 1903 | 1%-2% | 2%-4% | 6%-8% | 8%-10% | 0%-1% |
| Ex 1904 | 1%-2% | 2%-4% | 6%-8% | 8%-10% | 1%-2% |
| Ex 1905 | 1%-2% | 2%-4% | 6%-8% | 8%-10% | 2%-4% |
| Ex 1906 | 1%-2% | 2%-4% | 6%-8% | 8%-10% | 4%-6% |
| Ex 1907 | 1%-2% | 2%-4% | 6%-8% | 8%-10% | 6%-8% |
| Ex 1908 | 1%-2% | 2%-4% | 6%-8% | 8%-10% | 8%-10% |
| Ex 1909 | 1%-2% | 2%-4% | 8%-10% | 0%-1% | 0%-1% |
| Ex 1910 | 1%-2% | 2%-4% | 8%-10% | 0%-1% | 1%-2% |
| Ex 1911 | 1%-2% | 2%-4% | 8%-10% | 0%-1% | 2%-4% |
| Ex 1912 | 1%-2% | 2%-4% | 8%-10% | 0%-1% | 4%-6% |
| Ex 1913 | 1%-2% | 2%-4% | 8%-10% | 0%-1% | 6%-8% |
| Ex 1914 | 1%-2% | 2%-4% | 8%-10% | 0%-1% | 8%-10% |
| Ex 1915 | 1%-2% | 2%-4% | 8%-10% | 1%-2% | 0%-1% |
| Ex 1916 | 1%-2% | 2%-4% | 8%-10% | 1%-2% | 1%-2% |
| Ex 1917 | 1%-2% | 2%-4% | 8%-10% | 1%-2% | 2%-4% |
| Ex 1918 | 1%-2% | 2%-4% | 8%-10% | 1%-2% | 4%-6% |
| Ex 1919 | 1%-2% | 2%-4% | 8%-10% | 1%-2% | 6%-8% |
| Ex 1920 | 1%-2% | 2%-4% | 8%-10% | 1%-2% | 8%-10% |
| Ex 1921 | 1%-2% | 2%-4% | 8%-10% | 2%-4% | 0%-1% |
| Ex 1922 | 1%-2% | 2%-4% | 8%-10% | 2%-4% | 1%-2% |
| Ex 1923 | 1%-2% | 2%-4% | 8%-10% | 2%-4% | 2%-4% |
| Ex 1924 | 1%-2% | 2%-4% | 8%-10% | 2%-4% | 4%-6% |
| Ex 1925 | 1%-2% | 2%-4% | 8%-10% | 2%-4% | 6%-8% |
| Ex 1926 | 1%-2% | 2%-4% | 8%-10% | 2%-4% | 8%-10% |
| Ex 1927 | 1%-2% | 2%-4% | 8%-10% | 4%-6% | 0%-1% |
| Ex 1928 | 1%-2% | 2%-4% | 8%-10% | 4%-6% | 1%-2% |
| Ex 1929 | 1%-2% | 2%-4% | 8%-10% | 4%-6% | 2%-4% |
| Ex 1930 | 1%-2% | 2%-4% | 8%-10% | 4%-6% | 4%-6% |
| Ex 1931 | 1%-2% | 2%-4% | 8%-10% | 4%-6% | 6%-8% |
| Ex 1932 | 1%-2% | 2%-4% | 8%-10% | 4%-6% | 8%-10% |
| Ex 1933 | 1%-2% | 2%-4% | 8%-10% | 6%-8% | 0%-1% |
| Ex 1934 | 1%-2% | 2%-4% | 8%-10% | 6%-8% | 1%-2% |
| Ex 1935 | 1%-2% | 2%-4% | 8%-10% | 6%-8% | 2%-4% |
| Ex 1936 | 1%-2% | 2%-4% | 8%-10% | 6%-8% | 4%-6% |
| Ex 1937 | 1%-2% | 2%-4% | 8%-10% | 6%-8% | 6%-8% |
| Ex 1938 | 1%-2% | 2%-4% | 8%-10% | 6%-8% | 8%-10% |
| Ex 1939 | 1%-2% | 2%-4% | 8%-10% | 8%-10% | 0%-1% |
| Ex 1940 | 1%-2% | 2%-4% | 8%-10% | 8%-10% | 1%-2% |
| Ex 1941 | 1%-2% | 2%-4% | 8%-10% | 8%-10% | 2%-4% |
| Ex 1942 | 1%-2% | 2%-4% | 8%-10% | 8%-10% | 4%-6% |
| Ex 1943 | 1%-2% | 2%-4% | 8%-10% | 8%-10% | 6%-8% |
| Ex 1944 | 1%-2% | 2%-4% | 8%-10% | 8%-10% | 8%-10% |
| Ex 1945 | 1%-2% | 4%-6% | 0%-1% | 0%-1% | 0%-1% |
| Ex 1946 | 1%-2% | 4%-6% | 0%-1% | 0%-1% | 1%-2% |
| Ex 1947 | 1%-2% | 4%-6% | 0%-1% | 0%-1% | 2%-4% |
| Ex 1948 | 1%-2% | 4%-6% | 0%-1% | 0%-1% | 4%-6% |
| Ex 1949 | 1%-2% | 4%-6% | 0%-1% | 0%-1% | 6%-8% |
| Ex 1950 | 1%-2% | 4%-6% | 0%-1% | 0%-1% | 8%-10% |
| Ex 1951 | 1%-2% | 4%-6% | 0%-1% | 1%-2% | 0%-1% |
| Ex 1952 | 1%-2% | 4%-6% | 0%-1% | 1%-2% | 1%-2% |
| Ex 1953 | 1%-2% | 4%-6% | 0%-1% | 1%-2% | 2%-4% |
| Ex 1954 | 1%-2% | 4%-6% | 0%-1% | 1%-2% | 4%-6% |
| Ex 1955 | 1%-2% | 4%-6% | 0%-1% | 1%-2% | 6%-8% |
| Ex 1956 | 1%-2% | 4%-6% | 0%-1% | 1%-2% | 8%-10% |
| Ex 1957 | 1%-2% | 4%-6% | 0%-1% | 2%-4% | 0%-1% |
| Ex 1958 | 1%-2% | 4%-6% | 0%-1% | 2%-4% | 1%-2% |
| Ex 1959 | 1%-2% | 4%-6% | 0%-1% | 2%-4% | 2%-4% |
| Ex 1960 | 1%-2% | 4%-6% | 0%-1% | 2%-4% | 4%-6% |
| Ex 1961 | 1%-2% | 4%-6% | 0%-1% | 2%-4% | 6%-8% |
| Ex 1962 | 1%-2% | 4%-6% | 0%-1% | 2%-4% | 8%-10% |
| Ex 1963 | 1%-2% | 4%-6% | 0%-1% | 4%-6% | 0%-1% |
| Ex 1964 | 1%-2% | 4%-6% | 0%-1% | 4%-6% | 1%-2% |
| Ex 1965 | 1%-2% | 4%-6% | 0%-1% | 4%-6% | 2%-4% |
| Ex 1966 | 1%-2% | 4%-6% | 0%-1% | 4%-6% | 4%-6% |
| Ex 1967 | 1%-2% | 4%-6% | 0%-1% | 4%-6% | 6%-8% |
| Ex 1968 | 1%-2% | 4%-6% | 0%-1% | 4%-6% | 8%-10% |
| Ex 1969 | 1%-2% | 4%-6% | 0%-1% | 6%-8% | 0%-1% |
| Ex 1970 | 1%-2% | 4%-6% | 0%-1% | 6%-8% | 1%-2% |
| Ex 1971 | 1%-2% | 4%-6% | 0%-1% | 6%-8% | 2%-4% |
| Ex 1972 | 1%-2% | 4%-6% | 0%-1% | 6%-8% | 4%-6% |
| Ex 1973 | 1%-2% | 4%-6% | 0%-1% | 6%-8% | 6%-8% |
| Ex 1974 | 1%-2% | 4%-6% | 0%-1% | 6%-8% | 8%-10% |
| Ex 1975 | 1%-2% | 4%-6% | 0%-1% | 8%-10% | 0%-1% |
| Ex 1976 | 1%-2% | 4%-6% | 0%-1% | 8%-10% | 1%-2% |
| Ex 1977 | 1%-2% | 4%-6% | 0%-1% | 8%-10% | 2%-4% |
| Ex 1978 | 1%-2% | 4%-6% | 0%-1% | 8%-10% | 4%-6% |
| Ex 1979 | 1%-2% | 4%-6% | 0%-1% | 8%-10% | 6%-8% |
| Ex 1980 | 1%-2% | 4%-6% | 0%-1% | 8%-10% | 8%-10% |
| Ex 1981 | 1%-2% | 4%-6% | 1%-2% | 0%-1% | 0%-1% |
| Ex 1982 | 1%-2% | 4%-6% | 1%-2% | 0%-1% | 1%-2% |
| Ex 1983 | 1%-2% | 4%-6% | 1%-2% | 0%-1% | 2%-4% |
| Ex 1984 | 1%-2% | 4%-6% | 1%-2% | 0%-1% | 4%-6% |
| Ex 1985 | 1%-2% | 4%-6% | 1%-2% | 0%-1% | 6%-8% |
| Ex 1986 | 1%-2% | 4%-6% | 1%-2% | 0%-1% | 8%-10% |
| Ex 1987 | 1%-2% | 4%-6% | 1%-2% | 1%-2% | 0%-1% |
| Ex 1988 | 1%-2% | 4%-6% | 1%-2% | 1%-2% | 1%-2% |
| Ex 1989 | 1%-2% | 4%-6% | 1%-2% | 1%-2% | 2%-4% |
| Ex 1990 | 1%-2% | 4%-6% | 1%-2% | 1%-2% | 4%-6% |
| Ex 1991 | 1%-2% | 4%-6% | 1%-2% | 1%-2% | 6%-8% |
| Ex 1992 | 1%-2% | 4%-6% | 1%-2% | 1%-2% | 8%-10% |
| Ex 1993 | 1%-2% | 4%-6% | 1%-2% | 2%-4% | 0%-1% |
| Ex 1994 | 1%-2% | 4%-6% | 1%-2% | 2%-4% | 1%-2% |
| Ex 1995 | 1%-2% | 4%-6% | 1%-2% | 2%-4% | 2%-4% |
| Ex 1996 | 1%-2% | 4%-6% | 1%-2% | 2%-4% | 4%-6% |
| Ex 1997 | 1%-2% | 4%-6% | 1%-2% | 2%-4% | 6%-8% |
| Ex 1998 | 1%-2% | 4%-6% | 1%-2% | 2%-4% | 8%-10% |
| Ex 1999 | 1%-2% | 4%-6% | 1%-2% | 4%-6% | 0%-1% |
| Ex 2000 | 1%-2% | 4%-6% | 1%-2% | 4%-6% | 1%-2% |
| Ex 2001 | 1%-2% | 4%-6% | 1%-2% | 4%-6% | 2%-4% |
| Ex 2002 | 1%-2% | 4%-6% | 1%-2% | 4%-6% | 4%-6% |
| Ex 2003 | 1%-2% | 4%-6% | 1%-2% | 4%-6% | 6%-8% |
| Ex 2004 | 1%-2% | 4%-6% | 1%-2% | 4%-6% | 8%-10% |
| Ex 2005 | 1%-2% | 4%-6% | 1%-2% | 6%-8% | 0%-1% |
| Ex 2006 | 1%-2% | 4%-6% | 1%-2% | 6%-8% | 1%-2% |
| Ex 2007 | 1%-2% | 4%-6% | 1%-2% | 6%-8% | 2%-4% |
| Ex 2008 | 1%-2% | 4%-6% | 1%-2% | 6%-8% | 4%-6% |
| Ex 2009 | 1%-2% | 4%-6% | 1%-2% | 6%-8% | 6%-8% |
| Ex 2010 | 1%-2% | 4%-6% | 1%-2% | 6%-8% | 8%-10% |
| Ex 2011 | 1%-2% | 4%-6% | 1%-2% | 8%-10% | 0%-1% |
| Ex 2012 | 1%-2% | 4%-6% | 1%-2% | 8%-10% | 1%-2% |
| Ex 2013 | 1%-2% | 4%-6% | 1%-2% | 8%-10% | 2%-4% |
| Ex 2014 | 1%-2% | 4%-6% | 1%-2% | 8%-10% | 4%-6% |
| Ex 2015 | 1%-2% | 4%-6% | 1%-2% | 8%-10% | 6%-8% |
| Ex 2016 | 1%-2% | 4%-6% | 1%-2% | 8%-10% | 8%-10% |
| Ex 2017 | 1%-2% | 4%-6% | 2%-4% | 0%-1% | 0%-1% |
| Ex 2018 | 1%-2% | 4%-6% | 2%-4% | 0%-1% | 1%-2% |
| Ex 2019 | 1%-2% | 4%-6% | 2%-4% | 0%-1% | 2%-4% |
| Ex 2020 | 1%-2% | 4%-6% | 2%-4% | 0%-1% | 4%-6% |
| Ex 2021 | 1%-2% | 4%-6% | 2%-4% | 0%-1% | 6%-8% |
| Ex 2022 | 1%-2% | 4%-6% | 2%-4% | 0%-1% | 8%-10% |
| Ex 2023 | 1%-2% | 4%-6% | 2%-4% | 1%-2% | 0%-1% |
| Ex 2024 | 1%-2% | 4%-6% | 2%-4% | 1%-2% | 1%-2% |
| Ex 2025 | 1%-2% | 4%-6% | 2%-4% | 1%-2% | 2%-4% |
| Ex 2026 | 1%-2% | 4%-6% | 2%-4% | 1%-2% | 4%-6% |
| Ex 2027 | 1%-2% | 4%-6% | 2%-4% | 1%-2% | 6%-8% |
| Ex 2028 | 1%-2% | 4%-6% | 2%-4% | 1%-2% | 8%-10% |
| Ex 2029 | 1%-2% | 4%-6% | 2%-4% | 2%-4% | 0%-1% |
| Ex 2030 | 1%-2% | 4%-6% | 2%-4% | 2%-4% | 1%-2% |
| Ex 2031 | 1%-2% | 4%-6% | 2%-4% | 2%-4% | 2%-4% |
| Ex 2032 | 1%-2% | 4%-6% | 2%-4% | 2%-4% | 4%-6% |
| Ex 2033 | 1%-2% | 4%-6% | 2%-4% | 2%-4% | 6%-8% |
| Ex 2034 | 1%-2% | 4%-6% | 2%-4% | 2%-4% | 8%-10% |
| Ex 2035 | 1%-2% | 4%-6% | 2%-4% | 4%-6% | 0%-1% |
| Ex 2036 | 1%-2% | 4%-6% | 2%-4% | 4%-6% | 1%-2% |
| Ex 2037 | 1%-2% | 4%-6% | 2%-4% | 4%-6% | 2%-4% |
| Ex 2038 | 1%-2% | 4%-6% | 2%-4% | 4%-6% | 4%-6% |
| Ex 2039 | 1%-2% | 4%-6% | 2%-4% | 4%-6% | 6%-8% |
| Ex 2040 | 1%-2% | 4%-6% | 2%-4% | 4%-6% | 8%-10% |
| Ex 2041 | 1%-2% | 4%-6% | 2%-4% | 6%-8% | 0%-1% |
| Ex 2042 | 1%-2% | 4%-6% | 2%-4% | 6%-8% | 1%-2% |
| Ex 2043 | 1%-2% | 4%-6% | 2%-4% | 6%-8% | 2%-4% |
| Ex 2044 | 1%-2% | 4%-6% | 2%-4% | 6%-8% | 4%-6% |
| Ex 2045 | 1%-2% | 4%-6% | 2%-4% | 6%-8% | 6%-8% |
| Ex 2046 | 1%-2% | 4%-6% | 2%-4% | 6%-8% | 8%-10% |
| Ex 2047 | 1%-2% | 4%-6% | 2%-4% | 8%-10% | 0%-1% |
| Ex 2048 | 1%-2% | 4%-6% | 2%-4% | 8%-10% | 1%-2% |
| Ex 2049 | 1%-2% | 4%-6% | 2%-4% | 8%-10% | 2%-4% |
| Ex 2050 | 1%-2% | 4%-6% | 2%-4% | 8%-10% | 4%-6% |
| Ex 2051 | 1%-2% | 4%-6% | 2%-4% | 8%-10% | 6%-8% |
| Ex 2052 | 1%-2% | 4%-6% | 2%-4% | 8%-10% | 8%-10% |
| Ex 2053 | 1%-2% | 4%-6% | 4%-6% | 0%-1% | 0%-1% |
| Ex 2054 | 1%-2% | 4%-6% | 4%-6% | 0%-1% | 1%-2% |
| Ex 2055 | 1%-2% | 4%-6% | 4%-6% | 0%-1% | 2%-4% |
| Ex 2056 | 1%-2% | 4%-6% | 4%-6% | 0%-1% | 4%-6% |
| Ex 2057 | 1%-2% | 4%-6% | 4%-6% | 0%-1% | 6%-8% |
| Ex 2058 | 1%-2% | 4%-6% | 4%-6% | 0%-1% | 8%-10% |
| Ex 2059 | 1%-2% | 4%-6% | 4%-6% | 1%-2% | 0%-1% |
| Ex 2060 | 1%-2% | 4%-6% | 4%-6% | 1%-2% | 1%-2% |
| Ex 2061 | 1%-2% | 4%-6% | 4%-6% | 1%-2% | 2%-4% |
| Ex 2062 | 1%-2% | 4%-6% | 4%-6% | 1%-2% | 4%-6% |
| Ex 2063 | 1%-2% | 4%-6% | 4%-6% | 1%-2% | 6%-8% |
| Ex 2064 | 1%-2% | 4%-6% | 4%-6% | 1%-2% | 8%-10% |
| Ex 2065 | 1%-2% | 4%-6% | 4%-6% | 2%-4% | 0%-1% |
| Ex 2066 | 1%-2% | 4%-6% | 4%-6% | 2%-4% | 1%-2% |
| Ex 2067 | 1%-2% | 4%-6% | 4%-6% | 2%-4% | 2%-4% |
| Ex 2068 | 1%-2% | 4%-6% | 4%-6% | 2%-4% | 4%-6% |
| Ex 2069 | 1%-2% | 4%-6% | 4%-6% | 2%-4% | 6%-8% |
| Ex 2070 | 1%-2% | 4%-6% | 4%-6% | 2%-4% | 8%-10% |
| Ex 2071 | 1%-2% | 4%-6% | 4%-6% | 4%-6% | 0%-1% |
| Ex 2072 | 1%-2% | 4%-6% | 4%-6% | 4%-6% | 1%-2% |
| Ex 2073 | 1%-2% | 4%-6% | 4%-6% | 4%-6% | 2%-4% |
| Ex 2074 | 1%-2% | 4%-6% | 4%-6% | 4%-6% | 4%-6% |
| Ex 2075 | 1%-2% | 4%-6% | 4%-6% | 4%-6% | 6%-8% |
| Ex 2076 | 1%-2% | 4%-6% | 4%-6% | 4%-6% | 8%-10% |
| Ex 2077 | 1%-2% | 4%-6% | 4%-6% | 6%-8% | 0%-1% |
| Ex 2078 | 1%-2% | 4%-6% | 4%-6% | 6%-8% | 1%-2% |
| Ex 2079 | 1%-2% | 4%-6% | 4%-6% | 6%-8% | 2%-4% |
| Ex 2080 | 1%-2% | 4%-6% | 4%-6% | 6%-8% | 4%-6% |
| Ex 2081 | 1%-2% | 4%-6% | 4%-6% | 6%-8% | 6%-8% |
| Ex 2082 | 1%-2% | 4%-6% | 4%-6% | 6%-8% | 8%-10% |
| Ex 2083 | 1%-2% | 4%-6% | 4%-6% | 8%-10% | 0%-1% |
| Ex 2084 | 1%-2% | 4%-6% | 4%-6% | 8%-10% | 1%-2% |
| Ex 2085 | 1%-2% | 4%-6% | 4%-6% | 8%-10% | 2%-4% |
| Ex 2086 | 1%-2% | 4%-6% | 4%-6% | 8%-10% | 4%-6% |
| Ex 2087 | 1%-2% | 4%-6% | 4%-6% | 8%-10% | 6%-8% |
| Ex 2088 | 1%-2% | 4%-6% | 4%-6% | 8%-10% | 8%-10% |
| Ex 2089 | 1%-2% | 4%-6% | 6%-8% | 0%-1% | 0%-1% |
| Ex 2090 | 1%-2% | 4%-6% | 6%-8% | 0%-1% | 1%-2% |
| Ex 2091 | 1%-2% | 4%-6% | 6%-8% | 0%-1% | 2%-4% |
| Ex 2092 | 1%-2% | 4%-6% | 6%-8% | 0%-1% | 4%-6% |
| Ex 2093 | 1%-2% | 4%-6% | 6%-8% | 0%-1% | 6%-8% |
| Ex 2094 | 1%-2% | 4%-6% | 6%-8% | 0%-1% | 8%-10% |
| Ex 2095 | 1%-2% | 4%-6% | 6%-8% | 1%-2% | 0%-1% |
| Ex 2096 | 1%-2% | 4%-6% | 6%-8% | 1%-2% | 1%-2% |
| Ex 2097 | 1%-2% | 4%-6% | 6%-8% | 1%-2% | 2%-4% |
| Ex 2098 | 1%-2% | 4%-6% | 6%-8% | 1%-2% | 4%-6% |
| Ex 2099 | 1%-2% | 4%-6% | 6%-8% | 1%-2% | 6%-8% |
| Ex 2100 | 1%-2% | 4%-6% | 6%-8% | 1%-2% | 8%-10% |
| Ex 2101 | 1%-2% | 4%-6% | 6%-8% | 2%-4% | 0%-1% |
| Ex 2102 | 1%-2% | 4%-6% | 6%-8% | 2%-4% | 1%-2% |
| Ex 2103 | 1%-2% | 4%-6% | 6%-8% | 2%-4% | 2%-4% |
| Ex 2104 | 1%-2% | 4%-6% | 6%-8% | 2%-4% | 4%-6% |
| Ex 2105 | 1%-2% | 4%-6% | 6%-8% | 2%-4% | 6%-8% |
| Ex 2106 | 1%-2% | 4%-6% | 6%-8% | 2%-4% | 8%-10% |
| Ex 2107 | 1%-2% | 4%-6% | 6%-8% | 4%-6% | 0%-1% |
| Ex 2108 | 1%-2% | 4%-6% | 6%-8% | 4%-6% | 1%-2% |
| Ex 2109 | 1%-2% | 4%-6% | 6%-8% | 4%-6% | 2%-4% |
| Ex 2110 | 1%-2% | 4%-6% | 6%-8% | 4%-6% | 4%-6% |
| Ex 2111 | 1%-2% | 4%-6% | 6%-8% | 4%-6% | 6%-8% |
| Ex 2112 | 1%-2% | 4%-6% | 6%-8% | 4%-6% | 8%-10% |
| Ex 2113 | 1%-2% | 4%-6% | 6%-8% | 6%-8% | 0%-1% |
| Ex 2114 | 1%-2% | 4%-6% | 6%-8% | 6%-8% | 1%-2% |
| Ex 2115 | 1%-2% | 4%-6% | 6%-8% | 6%-8% | 2%-4% |
| Ex 2116 | 1%-2% | 4%-6% | 6%-8% | 6%-8% | 4%-6% |
| Ex 2117 | 1%-2% | 4%-6% | 6%-8% | 6%-8% | 6%-8% |
| Ex 2118 | 1%-2% | 4%-6% | 6%-8% | 6%-8% | 8%-10% |
| Ex 2119 | 1%-2% | 4%-6% | 6%-8% | 8%-10% | 0%-1% |
| Ex 2120 | 1%-2% | 4%-6% | 6%-8% | 8%-10% | 1%-2% |
| Ex 2121 | 1%-2% | 4%-6% | 6%-8% | 8%-10% | 2%-4% |
| Ex 2122 | 1%-2% | 4%-6% | 6%-8% | 8%-10% | 4%-6% |
| Ex 2123 | 1%-2% | 4%-6% | 6%-8% | 8%-10% | 6%-8% |
| Ex 2124 | 1%-2% | 4%-6% | 6%-8% | 8%-10% | 8%-10% |
| Ex 2125 | 1%-2% | 4%-6% | 8%-10% | 0%-1% | 0%-1% |
| Ex 2126 | 1%-2% | 4%-6% | 8%-10% | 0%-1% | 1%-2% |
| Ex 2127 | 1%-2% | 4%-6% | 8%-10% | 0%-1% | 2%-4% |
| Ex 2128 | 1%-2% | 4%-6% | 8%-10% | 0%-1% | 4%-6% |
| Ex 2129 | 1%-2% | 4%-6% | 8%-10% | 0%-1% | 6%-8% |
| Ex 2130 | 1%-2% | 4%-6% | 8%-10% | 0%-1% | 8%-10% |
| Ex 2131 | 1%-2% | 4%-6% | 8%-10% | 1%-2% | 0%-1% |
| Ex 2132 | 1%-2% | 4%-6% | 8%-10% | 1%-2% | 1%-2% |
| Ex 2133 | 1%-2% | 4%-6% | 8%-10% | 1%-2% | 2%-4% |
| Ex 2134 | 1%-2% | 4%-6% | 8%-10% | 1%-2% | 4%-6% |
| Ex 2135 | 1%-2% | 4%-6% | 8%-10% | 1%-2% | 6%-8% |
| Ex 2136 | 1%-2% | 4%-6% | 8%-10% | 1%-2% | 8%-10% |
| Ex 2137 | 1%-2% | 4%-6% | 8%-10% | 2%-4% | 0%-1% |
| Ex 2138 | 1%-2% | 4%-6% | 8%-10% | 2%-4% | 1%-2% |
| Ex 2139 | 1%-2% | 4%-6% | 8%-10% | 2%-4% | 2%-4% |
| Ex 2140 | 1%-2% | 4%-6% | 8%-10% | 2%-4% | 4%-6% |
| Ex 2141 | 1%-2% | 4%-6% | 8%-10% | 2%-4% | 6%-8% |
| Ex 2142 | 1%-2% | 4%-6% | 8%-10% | 2%-4% | 8%-10% |
| Ex 2143 | 1%-2% | 4%-6% | 8%-10% | 4%-6% | 0%-1% |
| Ex 2144 | 1%-2% | 4%-6% | 8%-10% | 4%-6% | 1%-2% |
| Ex 2145 | 1%-2% | 4%-6% | 8%-10% | 4%-6% | 2%-4% |
| Ex 2146 | 1%-2% | 4%-6% | 8%-10% | 4%-6% | 4%-6% |
| Ex 2147 | 1%-2% | 4%-6% | 8%-10% | 4%-6% | 6%-8% |
| Ex 2148 | 1%-2% | 4%-6% | 8%-10% | 4%-6% | 8%-10% |
| Ex 2149 | 1%-2% | 4%-6% | 8%-10% | 6%-8% | 0%-1% |
| Ex 2150 | 1%-2% | 4%-6% | 8%-10% | 6%-8% | 1%-2% |
| Ex 2151 | 1%-2% | 4%-6% | 8%-10% | 6%-8% | 2%-4% |
| Ex 2152 | 1%-2% | 4%-6% | 8%-10% | 6%-8% | 4%-6% |
| Ex 2153 | 1%-2% | 4%-6% | 8%-10% | 6%-8% | 6%-8% |
| Ex 2154 | 1%-2% | 4%-6% | 8%-10% | 6%-8% | 8%-10% |
| Ex 2155 | 1%-2% | 4%-6% | 8%-10% | 8%-10% | 0%-1% |
| Ex 2156 | 1%-2% | 4%-6% | 8%-10% | 8%-10% | 1%-2% |
| Ex 2157 | 1%-2% | 4%-6% | 8%-10% | 8%-10% | 2%-4% |
| Ex 2158 | 1%-2% | 4%-6% | 8%-10% | 8%-10% | 4%-6% |
| Ex 2159 | 1%-2% | 4%-6% | 8%-10% | 8%-10% | 6%-8% |
| Ex 2160 | 1%-2% | 4%-6% | 8%-10% | 8%-10% | 8%-10% |
| Ex 2161 | 1%-2% | 6%-8% | 0%-1% | 0%-1% | 0%-1% |
| Ex 2162 | 1%-2% | 6%-8% | 0%-1% | 0%-1% | 1%-2% |
| Ex 2163 | 1%-2% | 6%-8% | 0%-1% | 0%-1% | 2%-4% |
| Ex 2164 | 1%-2% | 6%-8% | 0%-1% | 0%-1% | 4%-6% |
| Ex 2165 | 1%-2% | 6%-8% | 0%-1% | 0%-1% | 6%-8% |
| Ex 2166 | 1%-2% | 6%-8% | 0%-1% | 0%-1% | 8%-10% |
| Ex 2167 | 1%-2% | 6%-8% | 0%-1% | 1%-2% | 0%-1% |
| Ex 2168 | 1%-2% | 6%-8% | 0%-1% | 1%-2% | 1%-2% |
| Ex 2169 | 1%-2% | 6%-8% | 0%-1% | 1%-2% | 2%-4% |
| Ex 2170 | 1%-2% | 6%-8% | 0%-1% | 1%-2% | 4%-6% |
| Ex 2171 | 1%-2% | 6%-8% | 0%-1% | 1%-2% | 6%-8% |
| Ex 2172 | 1%-2% | 6%-8% | 0%-1% | 1%-2% | 8%-10% |
| Ex 2173 | 1%-2% | 6%-8% | 0%-1% | 2%-4% | 0%-1% |
| Ex 2174 | 1%-2% | 6%-8% | 0%-1% | 2%-4% | 1%-2% |
| Ex 2175 | 1%-2% | 6%-8% | 0%-1% | 2%-4% | 2%-4% |
| Ex 2176 | 1%-2% | 6%-8% | 0%-1% | 2%-4% | 4%-6% |
| Ex 2177 | 1%-2% | 6%-8% | 0%-1% | 2%-4% | 6%-8% |
| Ex 2178 | 1%-2% | 6%-8% | 0%-1% | 2%-4% | 8%-10% |
| Ex 2179 | 1%-2% | 6%-8% | 0%-1% | 4%-6% | 0%-1% |
| Ex 2180 | 1%-2% | 6%-8% | 0%-1% | 4%-6% | 1%-2% |
| Ex 2181 | 1%-2% | 6%-8% | 0%-1% | 4%-6% | 2%-4% |
| Ex 2182 | 1%-2% | 6%-8% | 0%-1% | 4%-6% | 4%-6% |
| Ex 2183 | 1%-2% | 6%-8% | 0%-1% | 4%-6% | 6%-8% |
| Ex 2184 | 1%-2% | 6%-8% | 0%-1% | 4%-6% | 8%-10% |
| Ex 2185 | 1%-2% | 6%-8% | 0%-1% | 6%-8% | 0%-1% |
| Ex 2186 | 1%-2% | 6%-8% | 0%-1% | 6%-8% | 1%-2% |
| Ex 2187 | 1%-2% | 6%-8% | 0%-1% | 6%-8% | 2%-4% |
| Ex 2188 | 1%-2% | 6%-8% | 0%-1% | 6%-8% | 4%-6% |
| Ex 2189 | 1%-2% | 6%-8% | 0%-1% | 6%-8% | 6%-8% |
| Ex 2190 | 1%-2% | 6%-8% | 0%-1% | 6%-8% | 8%-10% |
| Ex 2191 | 1%-2% | 6%-8% | 0%-1% | 8%-10% | 0%-1% |
| Ex 2192 | 1%-2% | 6%-8% | 0%-1% | 8%-10% | 1%-2% |
| Ex 2193 | 1%-2% | 6%-8% | 0%-1% | 8%-10% | 2%-4% |
| Ex 2194 | 1%-2% | 6%-8% | 0%-1% | 8%-10% | 4%-6% |
| Ex 2195 | 1%-2% | 6%-8% | 0%-1% | 8%-10% | 6%-8% |
| Ex 2196 | 1%-2% | 6%-8% | 0%-1% | 8%-10% | 8%-10% |
| Ex 2197 | 1%-2% | 6%-8% | 1%-2% | 0%-1% | 0%-1% |
| Ex 2198 | 1%-2% | 6%-8% | 1%-2% | 0%-1% | 1%-2% |
| Ex 2199 | 1%-2% | 6%-8% | 1%-2% | 0%-1% | 2%-4% |
| Ex 2200 | 1%-2% | 6%-8% | 1%-2% | 0%-1% | 4%-6% |
| Ex 2201 | 1%-2% | 6%-8% | 1%-2% | 0%-1% | 6%-8% |
| Ex 2202 | 1%-2% | 6%-8% | 1%-2% | 0%-1% | 8%-10% |
| Ex 2203 | 1%-2% | 6%-8% | 1%-2% | 1%-2% | 0%-1% |
| Ex 2204 | 1%-2% | 6%-8% | 1%-2% | 1%-2% | 1%-2% |
| Ex 2205 | 1%-2% | 6%-8% | 1%-2% | 1%-2% | 2%-4% |
| Ex 2206 | 1%-2% | 6%-8% | 1%-2% | 1%-2% | 4%-6% |
| Ex 2207 | 1%-2% | 6%-8% | 1%-2% | 1%-2% | 6%-8% |
| Ex 2208 | 1%-2% | 6%-8% | 1%-2% | 1%-2% | 8%-10% |
| Ex 2209 | 1%-2% | 6%-8% | 1%-2% | 2%-4% | 0%-1% |
| Ex 2210 | 1%-2% | 6%-8% | 1%-2% | 2%-4% | 1%-2% |
| Ex 2211 | 1%-2% | 6%-8% | 1%-2% | 2%-4% | 2%-4% |
| Ex 2212 | 1%-2% | 6%-8% | 1%-2% | 2%-4% | 4%-6% |
| Ex 2213 | 1%-2% | 6%-8% | 1%-2% | 2%-4% | 6%-8% |
| Ex 2214 | 1%-2% | 6%-8% | 1%-2% | 2%-4% | 8%-10% |
| Ex 2215 | 1%-2% | 6%-8% | 1%-2% | 4%-6% | 0%-1% |
| Ex 2216 | 1%-2% | 6%-8% | 1%-2% | 4%-6% | 1%-2% |
| Ex 2217 | 1%-2% | 6%-8% | 1%-2% | 4%-6% | 2%-4% |
| Ex 2218 | 1%-2% | 6%-8% | 1%-2% | 4%-6% | 4%-6% |
| Ex 2219 | 1%-2% | 6%-8% | 1%-2% | 4%-6% | 6%-8% |
| Ex 2220 | 1%-2% | 6%-8% | 1%-2% | 4%-6% | 8%-10% |
| Ex 2221 | 1%-2% | 6%-8% | 1%-2% | 6%-8% | 0%-1% |
| Ex 2222 | 1%-2% | 6%-8% | 1%-2% | 6%-8% | 1%-2% |
| Ex 2223 | 1%-2% | 6%-8% | 1%-2% | 6%-8% | 2%-4% |
| Ex 2224 | 1%-2% | 6%-8% | 1%-2% | 6%-8% | 4%-6% |
| Ex 2225 | 1%-2% | 6%-8% | 1%-2% | 6%-8% | 6%-8% |
| Ex 2226 | 1%-2% | 6%-8% | 1%-2% | 6%-8% | 8%-10% |
| Ex 2227 | 1%-2% | 6%-8% | 1%-2% | 8%-10% | 0%-1% |
| Ex 2228 | 1%-2% | 6%-8% | 1%-2% | 8%-10% | 1%-2% |
| Ex 2229 | 1%-2% | 6%-8% | 1%-2% | 8%-10% | 2%-4% |
| Ex 2230 | 1%-2% | 6%-8% | 1%-2% | 8%-10% | 4%-6% |
| Ex 2231 | 1%-2% | 6%-8% | 1%-2% | 8%-10% | 6%-8% |
| Ex 2232 | 1%-2% | 6%-8% | 1%-2% | 8%-10% | 8%-10% |
| Ex 2233 | 1%-2% | 6%-8% | 2%-4% | 0%-1% | 0%-1% |
| Ex 2234 | 1%-2% | 6%-8% | 2%-4% | 0%-1% | 1%-2% |
| Ex 2235 | 1%-2% | 6%-8% | 2%-4% | 0%-1% | 2%-4% |
| Ex 2236 | 1%-2% | 6%-8% | 2%-4% | 0%-1% | 4%-6% |
| Ex 2237 | 1%-2% | 6%-8% | 2%-4% | 0%-1% | 6%-8% |
| Ex 2238 | 1%-2% | 6%-8% | 2%-4% | 0%-1% | 8%-10% |
| Ex 2239 | 1%-2% | 6%-8% | 2%-4% | 1%-2% | 0%-1% |
| Ex 2240 | 1%-2% | 6%-8% | 2%-4% | 1%-2% | 1%-2% |
| Ex 2241 | 1%-2% | 6%-8% | 2%-4% | 1%-2% | 2%-4% |
| Ex 2242 | 1%-2% | 6%-8% | 2%-4% | 1%-2% | 4%-6% |
| Ex 2243 | 1%-2% | 6%-8% | 2%-4% | 1%-2% | 6%-8% |
| Ex 2244 | 1%-2% | 6%-8% | 2%-4% | 1%-2% | 8%-10% |
| Ex 2245 | 1%-2% | 6%-8% | 2%-4% | 2%-4% | 0%-1% |
| Ex 2246 | 1%-2% | 6%-8% | 2%-4% | 2%-4% | 1%-2% |
| Ex 2247 | 1%-2% | 6%-8% | 2%-4% | 2%-4% | 2%-4% |
| Ex 2248 | 1%-2% | 6%-8% | 2%-4% | 2%-4% | 4%-6% |
| Ex 2249 | 1%-2% | 6%-8% | 2%-4% | 2%-4% | 6%-8% |
| Ex 2250 | 1%-2% | 6%-8% | 2%-4% | 2%-4% | 8%-10% |
| Ex 2251 | 1%-2% | 6%-8% | 2%-4% | 4%-6% | 0%-1% |
| Ex 2252 | 1%-2% | 6%-8% | 2%-4% | 4%-6% | 1%-2% |
| Ex 2253 | 1%-2% | 6%-8% | 2%-4% | 4%-6% | 2%-4% |
| Ex 2254 | 1%-2% | 6%-8% | 2%-4% | 4%-6% | 4%-6% |
| Ex 2255 | 1%-2% | 6%-8% | 2%-4% | 4%-6% | 6%-8% |
| Ex 2256 | 1%-2% | 6%-8% | 2%-4% | 4%-6% | 8%-10% |
| Ex 2257 | 1%-2% | 6%-8% | 2%-4% | 6%-8% | 0%-1% |
| Ex 2258 | 1%-2% | 6%-8% | 2%-4% | 6%-8% | 1%-2% |
| Ex 2259 | 1%-2% | 6%-8% | 2%-4% | 6%-8% | 2%-4% |
| Ex 2260 | 1%-2% | 6%-8% | 2%-4% | 6%-8% | 4%-6% |
| Ex 2261 | 1%-2% | 6%-8% | 2%-4% | 6%-8% | 6%-8% |
| Ex 2262 | 1%-2% | 6%-8% | 2%-4% | 6%-8% | 8%-10% |
| Ex 2263 | 1%-2% | 6%-8% | 2%-4% | 8%-10% | 0%-1% |
| Ex 2264 | 1%-2% | 6%-8% | 2%-4% | 8%-10% | 1%-2% |
| Ex 2265 | 1%-2% | 6%-8% | 2%-4% | 8%-10% | 2%-4% |
| Ex 2266 | 1%-2% | 6%-8% | 2%-4% | 8%-10% | 4%-6% |
| Ex 2267 | 1%-2% | 6%-8% | 2%-4% | 8%-10% | 6%-8% |
| Ex 2268 | 1%-2% | 6%-8% | 2%-4% | 8%-10% | 8%-10% |
| Ex 2269 | 1%-2% | 6%-8% | 4%-6% | 0%-1% | 0%-1% |
| Ex 2270 | 1%-2% | 6%-8% | 4%-6% | 0%-1% | 1%-2% |
| Ex 2271 | 1%-2% | 6%-8% | 4%-6% | 0%-1% | 2%-4% |
| Ex 2272 | 1%-2% | 6%-8% | 4%-6% | 0%-1% | 4%-6% |
| Ex 2273 | 1%-2% | 6%-8% | 4%-6% | 0%-1% | 6%-8% |
| Ex 2274 | 1%-2% | 6%-8% | 4%-6% | 0%-1% | 8%-10% |
| Ex 2275 | 1%-2% | 6%-8% | 4%-6% | 1%-2% | 0%-1% |
| Ex 2276 | 1%-2% | 6%-8% | 4%-6% | 1%-2% | 1%-2% |
| Ex 2277 | 1%-2% | 6%-8% | 4%-6% | 1%-2% | 2%-4% |
| Ex 2278 | 1%-2% | 6%-8% | 4%-6% | 1%-2% | 4%-6% |
| Ex 2279 | 1%-2% | 6%-8% | 4%-6% | 1%-2% | 6%-8% |
| Ex 2280 | 1%-2% | 6%-8% | 4%-6% | 1%-2% | 8%-10% |
| Ex 2281 | 1%-2% | 6%-8% | 4%-6% | 2%-4% | 0%-1% |
| Ex 2282 | 1%-2% | 6%-8% | 4%-6% | 2%-4% | 1%-2% |
| Ex 2283 | 1%-2% | 6%-8% | 4%-6% | 2%-4% | 2%-4% |
| Ex 2284 | 1%-2% | 6%-8% | 4%-6% | 2%-4% | 4%-6% |
| Ex 2285 | 1%-2% | 6%-8% | 4%-6% | 2%-4% | 6%-8% |
| Ex 2286 | 1%-2% | 6%-8% | 4%-6% | 2%-4% | 8%-10% |
| Ex 2287 | 1%-2% | 6%-8% | 4%-6% | 4%-6% | 0%-1% |
| Ex 2288 | 1%-2% | 6%-8% | 4%-6% | 4%-6% | 1%-2% |
| Ex 2289 | 1%-2% | 6%-8% | 4%-6% | 4%-6% | 2%-4% |
| Ex 2290 | 1%-2% | 6%-8% | 4%-6% | 4%-6% | 4%-6% |
| Ex 2291 | 1%-2% | 6%-8% | 4%-6% | 4%-6% | 6%-8% |
| Ex 2292 | 1%-2% | 6%-8% | 4%-6% | 4%-6% | 8%-10% |
| Ex 2293 | 1%-2% | 6%-8% | 4%-6% | 6%-8% | 0%-1% |
| Ex 2294 | 1%-2% | 6%-8% | 4%-6% | 6%-8% | 1%-2% |
| Ex 2295 | 1%-2% | 6%-8% | 4%-6% | 6%-8% | 2%-4% |
| Ex 2296 | 1%-2% | 6%-8% | 4%-6% | 6%-8% | 4%-6% |
| Ex 2297 | 1%-2% | 6%-8% | 4%-6% | 6%-8% | 6%-8% |
| Ex 2298 | 1%-2% | 6%-8% | 4%-6% | 6%-8% | 8%-10% |
| Ex 2299 | 1%-2% | 6%-8% | 4%-6% | 8%-10% | 0%-1% |
| Ex 2300 | 1%-2% | 6%-8% | 4%-6% | 8%-10% | 1%-2% |
| Ex 2301 | 1%-2% | 6%-8% | 4%-6% | 8%-10% | 2%-4% |
| Ex 2302 | 1%-2% | 6%-8% | 4%-6% | 8%-10% | 4%-6% |
| Ex 2303 | 1%-2% | 6%-8% | 4%-6% | 8%-10% | 6%-8% |
| Ex 2304 | 1%-2% | 6%-8% | 4%-6% | 8%-10% | 8%-10% |
| Ex 2305 | 1%-2% | 6%-8% | 6%-8% | 0%-1% | 0%-1% |
| Ex 2306 | 1%-2% | 6%-8% | 6%-8% | 0%-1% | 1%-2% |
| Ex 2307 | 1%-2% | 6%-8% | 6%-8% | 0%-1% | 2%-4% |
| Ex 2308 | 1%-2% | 6%-8% | 6%-8% | 0%-1% | 4%-6% |
| Ex 2309 | 1%-2% | 6%-8% | 6%-8% | 0%-1% | 6%-8% |
| Ex 2310 | 1%-2% | 6%-8% | 6%-8% | 0%-1% | 8%-10% |
| Ex 2311 | 1%-2% | 6%-8% | 6%-8% | 1%-2% | 0%-1% |
| Ex 2312 | 1%-2% | 6%-8% | 6%-8% | 1%-2% | 1%-2% |
| Ex 2313 | 1%-2% | 6%-8% | 6%-8% | 1%-2% | 2%-4% |
| Ex 2314 | 1%-2% | 6%-8% | 6%-8% | 1%-2% | 4%-6% |
| Ex 2315 | 1%-2% | 6%-8% | 6%-8% | 1%-2% | 6%-8% |
| Ex 2316 | 1%-2% | 6%-8% | 6%-8% | 1%-2% | 8%-10% |
| Ex 2317 | 1%-2% | 6%-8% | 6%-8% | 2%-4% | 0%-1% |
| Ex 2318 | 1%-2% | 6%-8% | 6%-8% | 2%-4% | 1%-2% |
| Ex 2319 | 1%-2% | 6%-8% | 6%-8% | 2%-4% | 2%-4% |
| Ex 2320 | 1%-2% | 6%-8% | 6%-8% | 2%-4% | 4%-6% |
| Ex 2321 | 1%-2% | 6%-8% | 6%-8% | 2%-4% | 6%-8% |
| Ex 2322 | 1%-2% | 6%-8% | 6%-8% | 2%-4% | 8%-10% |
| Ex 2323 | 1%-2% | 6%-8% | 6%-8% | 4%-6% | 0%-1% |
| Ex 2324 | 1%-2% | 6%-8% | 6%-8% | 4%-6% | 1%-2% |
| Ex 2325 | 1%-2% | 6%-8% | 6%-8% | 4%-6% | 2%-4% |
| Ex 2326 | 1%-2% | 6%-8% | 6%-8% | 4%-6% | 4%-6% |
| Ex 2327 | 1%-2% | 6%-8% | 6%-8% | 4%-6% | 6%-8% |
| Ex 2328 | 1%-2% | 6%-8% | 6%-8% | 4%-6% | 8%-10% |
| Ex 2329 | 1%-2% | 6%-8% | 6%-8% | 6%-8% | 0%-1% |
| Ex 2330 | 1%-2% | 6%-8% | 6%-8% | 6%-8% | 1%-2% |
| Ex 2331 | 1%-2% | 6%-8% | 6%-8% | 6%-8% | 2%-4% |
| Ex 2332 | 1%-2% | 6%-8% | 6%-8% | 6%-8% | 4%-6% |
| Ex 2333 | 1%-2% | 6%-8% | 6%-8% | 6%-8% | 6%-8% |
| Ex 2334 | 1%-2% | 6%-8% | 6%-8% | 6%-8% | 8%-10% |
| Ex 2335 | 1%-2% | 6%-8% | 6%-8% | 8%-10% | 0%-1% |
| Ex 2336 | 1%-2% | 6%-8% | 6%-8% | 8%-10% | 1%-2% |
| Ex 2337 | 1%-2% | 6%-8% | 6%-8% | 8%-10% | 2%-4% |
| Ex 2338 | 1%-2% | 6%-8% | 6%-8% | 8%-10% | 4%-6% |
| Ex 2339 | 1%-2% | 6%-8% | 6%-8% | 8%-10% | 6%-8% |
| Ex 2340 | 1%-2% | 6%-8% | 6%-8% | 8%-10% | 8%-10% |
| Ex 2341 | 1%-2% | 6%-8% | 8%-10% | 0%-1% | 0%-1% |
| Ex 2342 | 1%-2% | 6%-8% | 8%-10% | 0%-1% | 1%-2% |
| Ex 2343 | 1%-2% | 6%-8% | 8%-10% | 0%-1% | 2%-4% |
| Ex 2344 | 1%-2% | 6%-8% | 8%-10% | 0%-1% | 4%-6% |
| Ex 2345 | 1%-2% | 6%-8% | 8%-10% | 0%-1% | 6%-8% |
| Ex 2346 | 1%-2% | 6%-8% | 8%-10% | 0%-1% | 8%-10% |
| Ex 2347 | 1%-2% | 6%-8% | 8%-10% | 1%-2% | 0%-1% |
| Ex 2348 | 1%-2% | 6%-8% | 8%-10% | 1%-2% | 1%-2% |
| Ex 2349 | 1%-2% | 6%-8% | 8%-10% | 1%-2% | 2%-4% |
| Ex 2350 | 1%-2% | 6%-8% | 8%-10% | 1%-2% | 4%-6% |
| Ex 2351 | 1%-2% | 6%-8% | 8%-10% | 1%-2% | 6%-8% |
| Ex 2352 | 1%-2% | 6%-8% | 8%-10% | 1%-2% | 8%-10% |
| Ex 2353 | 1%-2% | 6%-8% | 8%-10% | 2%-4% | 0%-1% |
| Ex 2354 | 1%-2% | 6%-8% | 8%-10% | 2%-4% | 1%-2% |
| Ex 2355 | 1%-2% | 6%-8% | 8%-10% | 2%-4% | 2%-4% |
| Ex 2356 | 1%-2% | 6%-8% | 8%-10% | 2%-4% | 4%-6% |
| Ex 2357 | 1%-2% | 6%-8% | 8%-10% | 2%-4% | 6%-8% |
| Ex 2358 | 1%-2% | 6%-8% | 8%-10% | 2%-4% | 8%-10% |
| Ex 2359 | 1%-2% | 6%-8% | 8%-10% | 4%-6% | 0%-1% |
| Ex 2360 | 1%-2% | 6%-8% | 8%-10% | 4%-6% | 1%-2% |
| Ex 2361 | 1%-2% | 6%-8% | 8%-10% | 4%-6% | 2%-4% |
| Ex 2362 | 1%-2% | 6%-8% | 8%-10% | 4%-6% | 4%-6% |
| Ex 2363 | 1%-2% | 6%-8% | 8%-10% | 4%-6% | 6%-8% |
| Ex 2364 | 1%-2% | 6%-8% | 8%-10% | 4%-6% | 8%-10% |
| Ex 2365 | 1%-2% | 6%-8% | 8%-10% | 6%-8% | 0%-1% |
| Ex 2366 | 1%-2% | 6%-8% | 8%-10% | 6%-8% | 1%-2% |
| Ex 2367 | 1%-2% | 6%-8% | 8%-10% | 6%-8% | 2%-4% |
| Ex 2368 | 1%-2% | 6%-8% | 8%-10% | 6%-8% | 4%-6% |
| Ex 2369 | 1%-2% | 6%-8% | 8%-10% | 6%-8% | 6%-8% |
| Ex 2370 | 1%-2% | 6%-8% | 8%-10% | 6%-8% | 8%-10% |
| Ex 2371 | 1%-2% | 6%-8% | 8%-10% | 8%-10% | 0%-1% |
| Ex 2372 | 1%-2% | 6%-8% | 8%-10% | 8%-10% | 1%-2% |
| Ex 2373 | 1%-2% | 6%-8% | 8%-10% | 8%-10% | 2%-4% |
| Ex 2374 | 1%-2% | 6%-8% | 8%-10% | 8%-10% | 4%-6% |
| Ex 2375 | 1%-2% | 6%-8% | 8%-10% | 8%-10% | 6%-8% |
| Ex 2376 | 1%-2% | 6%-8% | 8%-10% | 8%-10% | 8%-10% |
| Ex 2377 | 1%-2% | 8%-10% | 0%-1% | 0%-1% | 0%-1% |
| Ex 2378 | 1%-2% | 8%-10% | 0%-1% | 0%-1% | 1%-2% |
| Ex 2379 | 1%-2% | 8%-10% | 0%-1% | 0%-1% | 2%-4% |
| Ex 2380 | 1%-2% | 8%-10% | 0%-1% | 0%-1% | 4%-6% |
| Ex 2381 | 1%-2% | 8%-10% | 0%-1% | 0%-1% | 6%-8% |
| Ex 2382 | 1%-2% | 8%-10% | 0%-1% | 0%-1% | 8%-10% |
| Ex 2383 | 1%-2% | 8%-10% | 0%-1% | 1%-2% | 0%-1% |
| Ex 2384 | 1%-2% | 8%-10% | 0%-1% | 1%-2% | 1%-2% |
| Ex 2385 | 1%-2% | 8%-10% | 0%-1% | 1%-2% | 2%-4% |
| Ex 2386 | 1%-2% | 8%-10% | 0%-1% | 1%-2% | 4%-6% |
| Ex 2387 | 1%-2% | 8%-10% | 0%-1% | 1%-2% | 6%-8% |
| Ex 2388 | 1%-2% | 8%-10% | 0%-1% | 1%-2% | 8%-10% |
| Ex 2389 | 1%-2% | 8%-10% | 0%-1% | 2%-4% | 0%-1% |
| Ex 2390 | 1%-2% | 8%-10% | 0%-1% | 2%-4% | 1%-2% |
| Ex 2391 | 1%-2% | 8%-10% | 0%-1% | 2%-4% | 2%-4% |
| Ex 2392 | 1%-2% | 8%-10% | 0%-1% | 2%-4% | 4%-6% |
| Ex 2393 | 1%-2% | 8%-10% | 0%-1% | 2%-4% | 6%-8% |
| Ex 2394 | 1%-2% | 8%-10% | 0%-1% | 2%-4% | 8%-10% |
| Ex 2395 | 1%-2% | 8%-10% | 0%-1% | 4%-6% | 0%-1% |
| Ex 2396 | 1%-2% | 8%-10% | 0%-1% | 4%-6% | 1%-2% |
| Ex 2397 | 1%-2% | 8%-10% | 0%-1% | 4%-6% | 2%-4% |
| Ex 2398 | 1%-2% | 8%-10% | 0%-1% | 4%-6% | 4%-6% |
| Ex 2399 | 1%-2% | 8%-10% | 0%-1% | 4%-6% | 6%-8% |
| Ex 2400 | 1%-2% | 8%-10% | 0%-1% | 4%-6% | 8%-10% |
| Ex 2401 | 1%-2% | 8%-10% | 0%-1% | 6%-8% | 0%-1% |
| Ex 2402 | 1%-2% | 8%-10% | 0%-1% | 6%-8% | 1%-2% |
| Ex 2403 | 1%-2% | 8%-10% | 0%-1% | 6%-8% | 2%-4% |
| Ex 2404 | 1%-2% | 8%-10% | 0%-1% | 6%-8% | 4%-6% |
| Ex 2405 | 1%-2% | 8%-10% | 0%-1% | 6%-8% | 6%-8% |
| Ex 2406 | 1%-2% | 8%-10% | 0%-1% | 6%-8% | 8%-10% |
| Ex 2407 | 1%-2% | 8%-10% | 0%-1% | 8%-10% | 0%-1% |
| Ex 2408 | 1%-2% | 8%-10% | 0%-1% | 8%-10% | 1%-2% |
| Ex 2409 | 1%-2% | 8%-10% | 0%-1% | 8%-10% | 2%-4% |
| Ex 2410 | 1%-2% | 8%-10% | 0%-1% | 8%-10% | 4%-6% |
| Ex 2411 | 1%-2% | 8%-10% | 0%-1% | 8%-10% | 6%-8% |
| Ex 2412 | 1%-2% | 8%-10% | 0%-1% | 8%-10% | 8%-10% |
| Ex 2413 | 1%-2% | 8%-10% | 1%-2% | 0%-1% | 0%-1% |
| Ex 2414 | 1%-2% | 8%-10% | 1%-2% | 0%-1% | 1%-2% |
| Ex 2415 | 1%-2% | 8%-10% | 1%-2% | 0%-1% | 2%-4% |
| Ex 2416 | 1%-2% | 8%-10% | 1%-2% | 0%-1% | 4%-6% |
| Ex 2417 | 1%-2% | 8%-10% | 1%-2% | 0%-1% | 6%-8% |
| Ex 2418 | 1%-2% | 8%-10% | 1%-2% | 0%-1% | 8%-10% |
| Ex 2419 | 1%-2% | 8%-10% | 1%-2% | 1%-2% | 0%-1% |
| Ex 2420 | 1%-2% | 8%-10% | 1%-2% | 1%-2% | 1%-2% |
| Ex 2421 | 1%-2% | 8%-10% | 1%-2% | 1%-2% | 2%-4% |
| Ex 2422 | 1%-2% | 8%-10% | 1%-2% | 1%-2% | 4%-6% |
| Ex 2423 | 1%-2% | 8%-10% | 1%-2% | 1%-2% | 6%-8% |
| Ex 2424 | 1%-2% | 8%-10% | 1%-2% | 1%-2% | 8%-10% |
| Ex 2425 | 1%-2% | 8%-10% | 1%-2% | 2%-4% | 0%-1% |
| Ex 2426 | 1%-2% | 8%-10% | 1%-2% | 2%-4% | 1%-2% |
| Ex 2427 | 1%-2% | 8%-10% | 1%-2% | 2%-4% | 2%-4% |
| Ex 2428 | 1%-2% | 8%-10% | 1%-2% | 2%-4% | 4%-6% |
| Ex 2429 | 1%-2% | 8%-10% | 1%-2% | 2%-4% | 6%-8% |
| Ex 2430 | 1%-2% | 8%-10% | 1%-2% | 2%-4% | 8%-10% |
| Ex 2431 | 1%-2% | 8%-10% | 1%-2% | 4%-6% | 0%-1% |
| Ex 2432 | 1%-2% | 8%-10% | 1%-2% | 4%-6% | 1%-2% |
| Ex 2433 | 1%-2% | 8%-10% | 1%-2% | 4%-6% | 2%-4% |
| Ex 2434 | 1%-2% | 8%-10% | 1%-2% | 4%-6% | 4%-6% |
| Ex 2435 | 1%-2% | 8%-10% | 1%-2% | 4%-6% | 6%-8% |
| Ex 2436 | 1%-2% | 8%-10% | 1%-2% | 4%-6% | 8%-10% |
| Ex 2437 | 1%-2% | 8%-10% | 1%-2% | 6%-8% | 0%-1% |
| Ex 2438 | 1%-2% | 8%-10% | 1%-2% | 6%-8% | 1%-2% |
| Ex 2439 | 1%-2% | 8%-10% | 1%-2% | 6%-8% | 2%-4% |
| Ex 2440 | 1%-2% | 8%-10% | 1%-2% | 6%-8% | 4%-6% |
| Ex 2441 | 1%-2% | 8%-10% | 1%-2% | 6%-8% | 6%-8% |
| Ex 2442 | 1%-2% | 8%-10% | 1%-2% | 6%-8% | 8%-10% |
| Ex 2443 | 1%-2% | 8%-10% | 1%-2% | 8%-10% | 0%-1% |
| Ex 2444 | 1%-2% | 8%-10% | 1%-2% | 8%-10% | 1%-2% |
| Ex 2445 | 1%-2% | 8%-10% | 1%-2% | 8%-10% | 2%-4% |
| Ex 2446 | 1%-2% | 8%-10% | 1%-2% | 8%-10% | 4%-6% |
| Ex 2447 | 1%-2% | 8%-10% | 1%-2% | 8%-10% | 6%-8% |
| Ex 2448 | 1%-2% | 8%-10% | 1%-2% | 8%-10% | 8%-10% |
| Ex 2449 | 1%-2% | 8%-10% | 2%-4% | 0%-1% | 0%-1% |
| Ex 2450 | 1%-2% | 8%-10% | 2%-4% | 0%-1% | 1%-2% |
| Ex 2451 | 1%-2% | 8%-10% | 2%-4% | 0%-1% | 2%-4% |
| Ex 2452 | 1%-2% | 8%-10% | 2%-4% | 0%-1% | 4%-6% |
| Ex 2453 | 1%-2% | 8%-10% | 2%-4% | 0%-1% | 6%-8% |
| Ex 2454 | 1%-2% | 8%-10% | 2%-4% | 0%-1% | 8%-10% |
| Ex 2455 | 1%-2% | 8%-10% | 2%-4% | 1%-2% | 0%-1% |
| Ex 2456 | 1%-2% | 8%-10% | 2%-4% | 1%-2% | 1%-2% |
| Ex 2457 | 1%-2% | 8%-10% | 2%-4% | 1%-2% | 2%-4% |
| Ex 2458 | 1%-2% | 8%-10% | 2%-4% | 1%-2% | 4%-6% |
| Ex 2459 | 1%-2% | 8%-10% | 2%-4% | 1%-2% | 6%-8% |
| Ex 2460 | 1%-2% | 8%-10% | 2%-4% | 1%-2% | 8%-10% |
| Ex 2461 | 1%-2% | 8%-10% | 2%-4% | 2%-4% | 0%-1% |
| Ex 2462 | 1%-2% | 8%-10% | 2%-4% | 2%-4% | 1%-2% |
| Ex 2463 | 1%-2% | 8%-10% | 2%-4% | 2%-4% | 2%-4% |
| Ex 2464 | 1%-2% | 8%-10% | 2%-4% | 2%-4% | 4%-6% |
| Ex 2465 | 1%-2% | 8%-10% | 2%-4% | 2%-4% | 6%-8% |
| Ex 2466 | 1%-2% | 8%-10% | 2%-4% | 2%-4% | 8%-10% |
| Ex 2467 | 1%-2% | 8%-10% | 2%-4% | 4%-6% | 0%-1% |
| Ex 2468 | 1%-2% | 8%-10% | 2%-4% | 4%-6% | 1%-2% |
| Ex 2469 | 1%-2% | 8%-10% | 2%-4% | 4%-6% | 2%-4% |
| Ex 2470 | 1%-2% | 8%-10% | 2%-4% | 4%-6% | 4%-6% |
| Ex 2471 | 1%-2% | 8%-10% | 2%-4% | 4%-6% | 6%-8% |
| Ex 2472 | 1%-2% | 8%-10% | 2%-4% | 4%-6% | 8%-10% |
| Ex 2473 | 1%-2% | 8%-10% | 2%-4% | 6%-8% | 0%-1% |
| Ex 2474 | 1%-2% | 8%-10% | 2%-4% | 6%-8% | 1%-2% |
| Ex 2475 | 1%-2% | 8%-10% | 2%-4% | 6%-8% | 2%-4% |
| Ex 2476 | 1%-2% | 8%-10% | 2%-4% | 6%-8% | 4%-6% |
| Ex 2477 | 1%-2% | 8%-10% | 2%-4% | 6%-8% | 6%-8% |
| Ex 2478 | 1%-2% | 8%-10% | 2%-4% | 6%-8% | 8%-10% |
| Ex 2479 | 1%-2% | 8%-10% | 2%-4% | 8%-10% | 0%-1% |
| Ex 2480 | 1%-2% | 8%-10% | 2%-4% | 8%-10% | 1%-2% |
| Ex 2481 | 1%-2% | 8%-10% | 2%-4% | 8%-10% | 2%-4% |
| Ex 2482 | 1%-2% | 8%-10% | 2%-4% | 8%-10% | 4%-6% |
| Ex 2483 | 1%-2% | 8%-10% | 2%-4% | 8%-10% | 6%-8% |
| Ex 2484 | 1%-2% | 8%-10% | 2%-4% | 8%-10% | 8%-10% |
| Ex 2485 | 1%-2% | 8%-10% | 4%-6% | 0%-1% | 0%-1% |
| Ex 2486 | 1%-2% | 8%-10% | 4%-6% | 0%-1% | 1%-2% |
| Ex 2487 | 1%-2% | 8%-10% | 4%-6% | 0%-1% | 2%-4% |
| Ex 2488 | 1%-2% | 8%-10% | 4%-6% | 0%-1% | 4%-6% |
| Ex 2489 | 1%-2% | 8%-10% | 4%-6% | 0%-1% | 6%-8% |
| Ex 2490 | 1%-2% | 8%-10% | 4%-6% | 0%-1% | 8%-10% |
| Ex 2491 | 1%-2% | 8%-10% | 4%-6% | 1%-2% | 0%-1% |
| Ex 2492 | 1%-2% | 8%-10% | 4%-6% | 1%-2% | 1%-2% |
| Ex 2493 | 1%-2% | 8%-10% | 4%-6% | 1%-2% | 2%-4% |
| Ex 2494 | 1%-2% | 8%-10% | 4%-6% | 1%-2% | 4%-6% |
| Ex 2495 | 1%-2% | 8%-10% | 4%-6% | 1%-2% | 6%-8% |
| Ex 2496 | 1%-2% | 8%-10% | 4%-6% | 1%-2% | 8%-10% |
| Ex 2497 | 1%-2% | 8%-10% | 4%-6% | 2%-4% | 0%-1% |
| Ex 2498 | 1%-2% | 8%-10% | 4%-6% | 2%-4% | 1%-2% |
| Ex 2499 | 1%-2% | 8%-10% | 4%-6% | 2%-4% | 2%-4% |
| Ex 2500 | 1%-2% | 8%-10% | 4%-6% | 2%-4% | 4%-6% |
| Ex 2501 | 1%-2% | 8%-10% | 4%-6% | 2%-4% | 6%-8% |
| Ex 2502 | 1%-2% | 8%-10% | 4%-6% | 2%-4% | 8%-10% |
| Ex 2503 | 1%-2% | 8%-10% | 4%-6% | 4%-6% | 0%-1% |
| Ex 2504 | 1%-2% | 8%-10% | 4%-6% | 4%-6% | 1%-2% |
| Ex 2505 | 1%-2% | 8%-10% | 4%-6% | 4%-6% | 2%-4% |
| Ex 2506 | 1%-2% | 8%-10% | 4%-6% | 4%-6% | 4%-6% |
| Ex 2507 | 1%-2% | 8%-10% | 4%-6% | 4%-6% | 6%-8% |
| Ex 2508 | 1%-2% | 8%-10% | 4%-6% | 4%-6% | 8%-10% |
| Ex 2509 | 1%-2% | 8%-10% | 4%-6% | 6%-8% | 0%-1% |
| Ex 2510 | 1%-2% | 8%-10% | 4%-6% | 6%-8% | 1%-2% |
| Ex 2511 | 1%-2% | 8%-10% | 4%-6% | 6%-8% | 2%-4% |
| Ex 2512 | 1%-2% | 8%-10% | 4%-6% | 6%-8% | 4%-6% |
| Ex 2513 | 1%-2% | 8%-10% | 4%-6% | 6%-8% | 6%-8% |
| Ex 2514 | 1%-2% | 8%-10% | 4%-6% | 6%-8% | 8%-10% |
| Ex 2515 | 1%-2% | 8%-10% | 4%-6% | 8%-10% | 0%-1% |
| Ex 2516 | 1%-2% | 8%-10% | 4%-6% | 8%-10% | 1%-2% |
| Ex 2517 | 1%-2% | 8%-10% | 4%-6% | 8%-10% | 2%-4% |
| Ex 2518 | 1%-2% | 8%-10% | 4%-6% | 8%-10% | 4%-6% |
| Ex 2519 | 1%-2% | 8%-10% | 4%-6% | 8%-10% | 6%-8% |
| Ex 2520 | 1%-2% | 8%-10% | 4%-6% | 8%-10% | 8%-10% |
| Ex 2521 | 1%-2% | 8%-10% | 6%-8% | 0%-1% | 0%-1% |
| Ex 2522 | 1%-2% | 8%-10% | 6%-8% | 0%-1% | 1%-2% |
| Ex 2523 | 1%-2% | 8%-10% | 6%-8% | 0%-1% | 2%-4% |
| Ex 2524 | 1%-2% | 8%-10% | 6%-8% | 0%-1% | 4%-6% |
| Ex 2525 | 1%-2% | 8%-10% | 6%-8% | 0%-1% | 6%-8% |
| Ex 2526 | 1%-2% | 8%-10% | 6%-8% | 0%-1% | 8%-10% |
| Ex 2527 | 1%-2% | 8%-10% | 6%-8% | 1%-2% | 0%-1% |
| Ex 2528 | 1%-2% | 8%-10% | 6%-8% | 1%-2% | 1%-2% |
| Ex 2529 | 1%-2% | 8%-10% | 6%-8% | 1%-2% | 2%-4% |
| Ex 2530 | 1%-2% | 8%-10% | 6%-8% | 1%-2% | 4%-6% |
| Ex 2531 | 1%-2% | 8%-10% | 6%-8% | 1%-2% | 6%-8% |
| Ex 2532 | 1%-2% | 8%-10% | 6%-8% | 1%-2% | 8%-10% |
| Ex 2533 | 1%-2% | 8%-10% | 6%-8% | 2%-4% | 0%-1% |
| Ex 2534 | 1%-2% | 8%-10% | 6%-8% | 2%-4% | 1%-2% |
| Ex 2535 | 1%-2% | 8%-10% | 6%-8% | 2%-4% | 2%-4% |
| Ex 2536 | 1%-2% | 8%-10% | 6%-8% | 2%-4% | 4%-6% |
| Ex 2537 | 1%-2% | 8%-10% | 6%-8% | 2%-4% | 6%-8% |
| Ex 2538 | 1%-2% | 8%-10% | 6%-8% | 2%-4% | 8%-10% |
| Ex 2539 | 1%-2% | 8%-10% | 6%-8% | 4%-6% | 0%-1% |
| Ex 2540 | 1%-2% | 8%-10% | 6%-8% | 4%-6% | 1%-2% |
| Ex 2541 | 1%-2% | 8%-10% | 6%-8% | 4%-6% | 2%-4% |
| Ex 2542 | 1%-2% | 8%-10% | 6%-8% | 4%-6% | 4%-6% |
| Ex 2543 | 1%-2% | 8%-10% | 6%-8% | 4%-6% | 6%-8% |
| Ex 2544 | 1%-2% | 8%-10% | 6%-8% | 4%-6% | 8%-10% |
| Ex 2545 | 1%-2% | 8%-10% | 6%-8% | 6%-8% | 0%-1% |
| Ex 2546 | 1%-2% | 8%-10% | 6%-8% | 6%-8% | 1%-2% |
| Ex 2547 | 1%-2% | 8%-10% | 6%-8% | 6%-8% | 2%-4% |
| Ex 2548 | 1%-2% | 8%-10% | 6%-8% | 6%-8% | 4%-6% |
| Ex 2549 | 1%-2% | 8%-10% | 6%-8% | 6%-8% | 6%-8% |
| Ex 2550 | 1%-2% | 8%-10% | 6%-8% | 6%-8% | 8%-10% |
| Ex 2551 | 1%-2% | 8%-10% | 6%-8% | 8%-10% | 0%-1% |
| Ex 2552 | 1%-2% | 8%-10% | 6%-8% | 8%-10% | 1%-2% |
| Ex 2553 | 1%-2% | 8%-10% | 6%-8% | 8%-10% | 2%-4% |
| Ex 2554 | 1%-2% | 8%-10% | 6%-8% | 8%-10% | 4%-6% |
| Ex 2555 | 1%-2% | 8%-10% | 6%-8% | 8%-10% | 6%-8% |
| Ex 2556 | 1%-2% | 8%-10% | 6%-8% | 8%-10% | 8%-10% |
| Ex 2557 | 1%-2% | 8%-10% | 8%-10% | 0%-1% | 0%-1% |
| Ex 2558 | 1%-2% | 8%-10% | 8%-10% | 0%-1% | 1%-2% |
| Ex 2559 | 1%-2% | 8%-10% | 8%-10% | 0%-1% | 2%-4% |
| Ex 2560 | 1%-2% | 8%-10% | 8%-10% | 0%-1% | 4%-6% |
| Ex 2561 | 1%-2% | 8%-10% | 8%-10% | 0%-1% | 6%-8% |
| Ex 2562 | 1%-2% | 8%-10% | 8%-10% | 0%-1% | 8%-10% |
| Ex 2563 | 1%-2% | 8%-10% | 8%-10% | 1%-2% | 0%-1% |
| Ex 2564 | 1%-2% | 8%-10% | 8%-10% | 1%-2% | 1%-2% |
| Ex 2565 | 1%-2% | 8%-10% | 8%-10% | 1%-2% | 2%-4% |
| Ex 2566 | 1%-2% | 8%-10% | 8%-10% | 1%-2% | 4%-6% |
| Ex 2567 | 1%-2% | 8%-10% | 8%-10% | 1%-2% | 6%-8% |
| Ex 2568 | 1%-2% | 8%-10% | 8%-10% | 1%-2% | 8%-10% |
| Ex 2569 | 1%-2% | 8%-10% | 8%-10% | 2%-4% | 0%-1% |
| Ex 2570 | 1%-2% | 8%-10% | 8%-10% | 2%-4% | 1%-2% |
| Ex 2571 | 1%-2% | 8%-10% | 8%-10% | 2%-4% | 2%-4% |
| Ex 2572 | 1%-2% | 8%-10% | 8%-10% | 2%-4% | 4%-6% |
| Ex 2573 | 1%-2% | 8%-10% | 8%-10% | 2%-4% | 6%-8% |
| Ex 2574 | 1%-2% | 8%-10% | 8%-10% | 2%-4% | 8%-10% |
| Ex 2575 | 1%-2% | 8%-10% | 8%-10% | 4%-6% | 0%-1% |
| Ex 2576 | 1%-2% | 8%-10% | 8%-10% | 4%-6% | 1%-2% |
| Ex 2577 | 1%-2% | 8%-10% | 8%-10% | 4%-6% | 2%-4% |
| Ex 2578 | 1%-2% | 8%-10% | 8%-10% | 4%-6% | 4%-6% |
| Ex 2579 | 1%-2% | 8%-10% | 8%-10% | 4%-6% | 6%-8% |
| Ex 2580 | 1%-2% | 8%-10% | 8%-10% | 4%-6% | 8%-10% |
| Ex 2581 | 1%-2% | 8%-10% | 8%-10% | 6%-8% | 0%-1% |
| Ex 2582 | 1%-2% | 8%-10% | 8%-10% | 6%-8% | 1%-2% |
| Ex 2583 | 1%-2% | 8%-10% | 8%-10% | 6%-8% | 2%-4% |
| Ex 2584 | 1%-2% | 8%-10% | 8%-10% | 6%-8% | 4%-6% |
| Ex 2585 | 1%-2% | 8%-10% | 8%-10% | 6%-8% | 6%-8% |
| Ex 2586 | 1%-2% | 8%-10% | 8%-10% | 6%-8% | 8%-10% |
| Ex 2587 | 1%-2% | 8%-10% | 8%-10% | 8%-10% | 0%-1% |
| Ex 2588 | 1%-2% | 8%-10% | 8%-10% | 8%-10% | 1%-2% |
| Ex 2589 | 1%-2% | 8%-10% | 8%-10% | 8%-10% | 2%-4% |
| Ex 2590 | 1%-2% | 8%-10% | 8%-10% | 8%-10% | 4%-6% |
| Ex 2591 | 1%-2% | 8%-10% | 8%-10% | 8%-10% | 6%-8% |
| Ex 2592 | 1%-2% | 8%-10% | 8%-10% | 8%-10% | 8%-10% |
| Ex 2593 | 2%-4% | 0%-1% | 0%-1% | 0%-1% | 0%-1% |
| Ex 2594 | 2%-4% | 0%-1% | 0%-1% | 0%-1% | 1%-2% |
| Ex 2595 | 2%-4% | 0%-1% | 0%-1% | 0%-1% | 2%-4% |
| Ex 2596 | 2%-4% | 0%-1% | 0%-1% | 0%-1% | 4%-6% |
| Ex 2597 | 2%-4% | 0%-1% | 0%-1% | 0%-1% | 6%-8% |
| Ex 2598 | 2%-4% | 0%-1% | 0%-1% | 0%-1% | 8%-10% |
| Ex 2599 | 2%-4% | 0%-1% | 0%-1% | 1%-2% | 0%-1% |
| Ex 2600 | 2%-4% | 0%-1% | 0%-1% | 1%-2% | 1%-2% |
| Ex 2601 | 2%-4% | 0%-1% | 0%-1% | 1%-2% | 2%-4% |
| Ex 2602 | 2%-4% | 0%-1% | 0%-1% | 1%-2% | 4%-6% |
| Ex 2603 | 2%-4% | 0%-1% | 0%-1% | 1%-2% | 6%-8% |
| Ex 2604 | 2%-4% | 0%-1% | 0%-1% | 1%-2% | 8%-10% |
| Ex 2605 | 2%-4% | 0%-1% | 0%-1% | 2%-4% | 0%-1% |
| Ex 2606 | 2%-4% | 0%-1% | 0%-1% | 2%-4% | 1%-2% |
| Ex 2607 | 2%-4% | 0%-1% | 0%-1% | 2%-4% | 2%-4% |
| Ex 2608 | 2%-4% | 0%-1% | 0%-1% | 2%-4% | 4%-6% |
| Ex 2609 | 2%-4% | 0%-1% | 0%-1% | 2%-4% | 6%-8% |
| Ex 2610 | 2%-4% | 0%-1% | 0%-1% | 2%-4% | 8%-10% |
| Ex 2611 | 2%-4% | 0%-1% | 0%-1% | 4%-6% | 0%-1% |
| Ex 2612 | 2%-4% | 0%-1% | 0%-1% | 4%-6% | 1%-2% |
| Ex 2613 | 2%-4% | 0%-1% | 0%-1% | 4%-6% | 2%-4% |
| Ex 2614 | 2%-4% | 0%-1% | 0%-1% | 4%-6% | 4%-6% |
| Ex 2615 | 2%-4% | 0%-1% | 0%-1% | 4%-6% | 6%-8% |
| Ex 2616 | 2%-4% | 0%-1% | 0%-1% | 4%-6% | 8%-10% |
| Ex 2617 | 2%-4% | 0%-1% | 0%-1% | 6%-8% | 0%-1% |
| Ex 2618 | 2%-4% | 0%-1% | 0%-1% | 6%-8% | 1%-2% |
| Ex 2619 | 2%-4% | 0%-1% | 0%-1% | 6%-8% | 2%-4% |
| Ex 2620 | 2%-4% | 0%-1% | 0%-1% | 6%-8% | 4%-6% |
| Ex 2621 | 2%-4% | 0%-1% | 0%-1% | 6%-8% | 6%-8% |
| Ex 2622 | 2%-4% | 0%-1% | 0%-1% | 6%-8% | 8%-10% |
| Ex 2623 | 2%-4% | 0%-1% | 0%-1% | 8%-10% | 0%-1% |
| Ex 2624 | 2%-4% | 0%-1% | 0%-1% | 8%-10% | 1%-2% |
| Ex 2625 | 2%-4% | 0%-1% | 0%-1% | 8%-10% | 2%-4% |
| Ex 2626 | 2%-4% | 0%-1% | 0%-1% | 8%-10% | 4%-6% |
| Ex 2627 | 2%-4% | 0%-1% | 0%-1% | 8%-10% | 6%-8% |
| Ex 2628 | 2%-4% | 0%-1% | 0%-1% | 8%-10% | 8%-10% |
| Ex 2629 | 2%-4% | 0%-1% | 1%-2% | 0%-1% | 0%-1% |
| Ex 2630 | 2%-4% | 0%-1% | 1%-2% | 0%-1% | 1%-2% |
| Ex 2631 | 2%-4% | 0%-1% | 1%-2% | 0%-1% | 2%-4% |
| Ex 2632 | 2%-4% | 0%-1% | 1%-2% | 0%-1% | 4%-6% |
| Ex 2633 | 2%-4% | 0%-1% | 1%-2% | 0%-1% | 6%-8% |
| Ex 2634 | 2%-4% | 0%-1% | 1%-2% | 0%-1% | 8%-10% |
| Ex 2635 | 2%-4% | 0%-1% | 1%-2% | 1%-2% | 0%-1% |
| Ex 2636 | 2%-4% | 0%-1% | 1%-2% | 1%-2% | 1%-2% |
| Ex 2637 | 2%-4% | 0%-1% | 1%-2% | 1%-2% | 2%-4% |
| Ex 2638 | 2%-4% | 0%-1% | 1%-2% | 1%-2% | 4%-6% |
| Ex 2639 | 2%-4% | 0%-1% | 1%-2% | 1%-2% | 6%-8% |
| Ex 2640 | 2%-4% | 0%-1% | 1%-2% | 1%-2% | 8%-10% |
| Ex 2641 | 2%-4% | 0%-1% | 1%-2% | 2%-4% | 0%-1% |
| Ex 2642 | 2%-4% | 0%-1% | 1%-2% | 2%-4% | 1%-2% |
| Ex 2643 | 2%-4% | 0%-1% | 1%-2% | 2%-4% | 2%-4% |
| Ex 2644 | 2%-4% | 0%-1% | 1%-2% | 2%-4% | 4%-6% |
| Ex 2645 | 2%-4% | 0%-1% | 1%-2% | 2%-4% | 6%-8% |
| Ex 2646 | 2%-4% | 0%-1% | 1%-2% | 2%-4% | 8%-10% |
| Ex 2647 | 2%-4% | 0%-1% | 1%-2% | 4%-6% | 0%-1% |
| Ex 2648 | 2%-4% | 0%-1% | 1%-2% | 4%-6% | 1%-2% |
| Ex 2649 | 2%-4% | 0%-1% | 1%-2% | 4%-6% | 2%-4% |
| Ex 2650 | 2%-4% | 0%-1% | 1%-2% | 4%-6% | 4%-6% |
| Ex 2651 | 2%-4% | 0%-1% | 1%-2% | 4%-6% | 6%-8% |
| Ex 2652 | 2%-4% | 0%-1% | 1%-2% | 4%-6% | 8%-10% |
| Ex 2653 | 2%-4% | 0%-1% | 1%-2% | 6%-8% | 0%-1% |
| Ex 2654 | 2%-4% | 0%-1% | 1%-2% | 6%-8% | 1%-2% |
| Ex 2655 | 2%-4% | 0%-1% | 1%-2% | 6%-8% | 2%-4% |
| Ex 2656 | 2%-4% | 0%-1% | 1%-2% | 6%-8% | 4%-6% |
| Ex 2657 | 2%-4% | 0%-1% | 1%-2% | 6%-8% | 6%-8% |
| Ex 2658 | 2%-4% | 0%-1% | 1%-2% | 6%-8% | 8%-10% |
| Ex 2659 | 2%-4% | 0%-1% | 1%-2% | 8%-10% | 0%-1% |
| Ex 2660 | 2%-4% | 0%-1% | 1%-2% | 8%-10% | 1%-2% |
| Ex 2661 | 2%-4% | 0%-1% | 1%-2% | 8%-10% | 2%-4% |
| Ex 2662 | 2%-4% | 0%-1% | 1%-2% | 8%-10% | 4%-6% |
| Ex 2663 | 2%-4% | 0%-1% | 1%-2% | 8%-10% | 6%-8% |
| Ex 2664 | 2%-4% | 0%-1% | 1%-2% | 8%-10% | 8%-10% |
| Ex 2665 | 2%-4% | 0%-1% | 2%-4% | 0%-1% | 0%-1% |
| Ex 2666 | 2%-4% | 0%-1% | 2%-4% | 0%-1% | 1%-2% |
| Ex 2667 | 2%-4% | 0%-1% | 2%-4% | 0%-1% | 2%-4% |
| Ex 2668 | 2%-4% | 0%-1% | 2%-4% | 0%-1% | 4%-6% |
| Ex 2669 | 2%-4% | 0%-1% | 2%-4% | 0%-1% | 6%-8% |
| Ex 2670 | 2%-4% | 0%-1% | 2%-4% | 0%-1% | 8%-10% |
| Ex 2671 | 2%-4% | 0%-1% | 2%-4% | 1%-2% | 0%-1% |
| Ex 2672 | 2%-4% | 0%-1% | 2%-4% | 1%-2% | 1%-2% |
| Ex 2673 | 2%-4% | 0%-1% | 2%-4% | 1%-2% | 2%-4% |
| Ex 2674 | 2%-4% | 0%-1% | 2%-4% | 1%-2% | 4%-6% |
| Ex 2675 | 2%-4% | 0%-1% | 2%-4% | 1%-2% | 6%-8% |
| Ex 2676 | 2%-4% | 0%-1% | 2%-4% | 1%-2% | 8%-10% |
| Ex 2677 | 2%-4% | 0%-1% | 2%-4% | 2%-4% | 0%-1% |
| Ex 2678 | 2%-4% | 0%-1% | 2%-4% | 2%-4% | 1%-2% |
| Ex 2679 | 2%-4% | 0%-1% | 2%-4% | 2%-4% | 2%-4% |
| Ex 2680 | 2%-4% | 0%-1% | 2%-4% | 2%-4% | 4%-6% |
| Ex 2681 | 2%-4% | 0%-1% | 2%-4% | 2%-4% | 6%-8% |
| Ex 2682 | 2%-4% | 0%-1% | 2%-4% | 2%-4% | 8%-10% |
| Ex 2683 | 2%-4% | 0%-1% | 2%-4% | 4%-6% | 0%-1% |
| Ex 2684 | 2%-4% | 0%-1% | 2%-4% | 4%-6% | 1%-2% |
| Ex 2685 | 2%-4% | 0%-1% | 2%-4% | 4%-6% | 2%-4% |
| Ex 2686 | 2%-4% | 0%-1% | 2%-4% | 4%-6% | 4%-6% |
| Ex 2687 | 2%-4% | 0%-1% | 2%-4% | 4%-6% | 6%-8% |
| Ex 2688 | 2%-4% | 0%-1% | 2%-4% | 4%-6% | 8%-10% |
| Ex 2689 | 2%-4% | 0%-1% | 2%-4% | 6%-8% | 0%-1% |
| Ex 2690 | 2%-4% | 0%-1% | 2%-4% | 6%-8% | 1%-2% |
| Ex 2691 | 2%-4% | 0%-1% | 2%-4% | 6%-8% | 2%-4% |
| Ex 2692 | 2%-4% | 0%-1% | 2%-4% | 6%-8% | 4%-6% |
| Ex 2693 | 2%-4% | 0%-1% | 2%-4% | 6%-8% | 6%-8% |
| Ex 2694 | 2%-4% | 0%-1% | 2%-4% | 6%-8% | 8%-10% |
| Ex 2695 | 2%-4% | 0%-1% | 2%-4% | 8%-10% | 0%-1% |
| Ex 2696 | 2%-4% | 0%-1% | 2%-4% | 8%-10% | 1%-2% |
| Ex 2697 | 2%-4% | 0%-1% | 2%-4% | 8%-10% | 2%-4% |
| Ex 2698 | 2%-4% | 0%-1% | 2%-4% | 8%-10% | 4%-6% |
| Ex 2699 | 2%-4% | 0%-1% | 2%-4% | 8%-10% | 6%-8% |
| Ex 2700 | 2%-4% | 0%-1% | 2%-4% | 8%-10% | 8%-10% |
| Ex 2701 | 2%-4% | 0%-1% | 4%-6% | 0%-1% | 0%-1% |
| Ex 2702 | 2%-4% | 0%-1% | 4%-6% | 0%-1% | 1%-2% |
| Ex 2703 | 2%-4% | 0%-1% | 4%-6% | 0%-1% | 2%-4% |
| Ex 2704 | 2%-4% | 0%-1% | 4%-6% | 0%-1% | 4%-6% |
| Ex 2705 | 2%-4% | 0%-1% | 4%-6% | 0%-1% | 6%-8% |
| Ex 2706 | 2%-4% | 0%-1% | 4%-6% | 0%-1% | 8%-10% |
| Ex 2707 | 2%-4% | 0%-1% | 4%-6% | 1%-2% | 0%-1% |
| Ex 2708 | 2%-4% | 0%-1% | 4%-6% | 1%-2% | 1%-2% |
| Ex 2709 | 2%-4% | 0%-1% | 4%-6% | 1%-2% | 2%-4% |
| Ex 2710 | 2%-4% | 0%-1% | 4%-6% | 1%-2% | 4%-6% |
| Ex 2711 | 2%-4% | 0%-1% | 4%-6% | 1%-2% | 6%-8% |
| Ex 2712 | 2%-4% | 0%-1% | 4%-6% | 1%-2% | 8%-10% |
| Ex 2713 | 2%-4% | 0%-1% | 4%-6% | 2%-4% | 0%-1% |
| Ex 2714 | 2%-4% | 0%-1% | 4%-6% | 2%-4% | 1%-2% |
| Ex 2715 | 2%-4% | 0%-1% | 4%-6% | 2%-4% | 2%-4% |
| Ex 2716 | 2%-4% | 0%-1% | 4%-6% | 2%-4% | 4%-6% |
| Ex 2717 | 2%-4% | 0%-1% | 4%-6% | 2%-4% | 6%-8% |
| Ex 2718 | 2%-4% | 0%-1% | 4%-6% | 2%-4% | 8%-10% |
| Ex 2719 | 2%-4% | 0%-1% | 4%-6% | 4%-6% | 0%-1% |
| Ex 2720 | 2%-4% | 0%-1% | 4%-6% | 4%-6% | 1%-2% |
| Ex 2721 | 2%-4% | 0%-1% | 4%-6% | 4%-6% | 2%-4% |
| Ex 2722 | 2%-4% | 0%-1% | 4%-6% | 4%-6% | 4%-6% |
| Ex 2723 | 2%-4% | 0%-1% | 4%-6% | 4%-6% | 6%-8% |
| Ex 2724 | 2%-4% | 0%-1% | 4%-6% | 4%-6% | 8%-10% |
| Ex 2725 | 2%-4% | 0%-1% | 4%-6% | 6%-8% | 0%-1% |
| Ex 2726 | 2%-4% | 0%-1% | 4%-6% | 6%-8% | 1%-2% |
| Ex 2727 | 2%-4% | 0%-1% | 4%-6% | 6%-8% | 2%-4% |
| Ex 2728 | 2%-4% | 0%-1% | 4%-6% | 6%-8% | 4%-6% |
| Ex 2729 | 2%-4% | 0%-1% | 4%-6% | 6%-8% | 6%-8% |
| Ex 2730 | 2%-4% | 0%-1% | 4%-6% | 6%-8% | 8%-10% |
| Ex 2731 | 2%-4% | 0%-1% | 4%-6% | 8%-10% | 0%-1% |
| Ex 2732 | 2%-4% | 0%-1% | 4%-6% | 8%-10% | 1%-2% |
| Ex 2733 | 2%-4% | 0%-1% | 4%-6% | 8%-10% | 2%-4% |
| Ex 2734 | 2%-4% | 0%-1% | 4%-6% | 8%-10% | 4%-6% |
| Ex 2735 | 2%-4% | 0%-1% | 4%-6% | 8%-10% | 6%-8% |
| Ex 2736 | 2%-4% | 0%-1% | 4%-6% | 8%-10% | 8%-10% |
| Ex 2737 | 2%-4% | 0%-1% | 6%-8% | 0%-1% | 0%-1% |
| Ex 2738 | 2%-4% | 0%-1% | 6%-8% | 0%-1% | 1%-2% |
| Ex 2739 | 2%-4% | 0%-1% | 6%-8% | 0%-1% | 2%-4% |
| Ex 2740 | 2%-4% | 0%-1% | 6%-8% | 0%-1% | 4%-6% |
| Ex 2741 | 2%-4% | 0%-1% | 6%-8% | 0%-1% | 6%-8% |
| Ex 2742 | 2%-4% | 0%-1% | 6%-8% | 0%-1% | 8%-10% |
| Ex 2743 | 2%-4% | 0%-1% | 6%-8% | 1%-2% | 0%-1% |
| Ex 2744 | 2%-4% | 0%-1% | 6%-8% | 1%-2% | 1%-2% |
| Ex 2745 | 2%-4% | 0%-1% | 6%-8% | 1%-2% | 2%-4% |
| Ex 2746 | 2%-4% | 0%-1% | 6%-8% | 1%-2% | 4%-6% |
| Ex 2747 | 2%-4% | 0%-1% | 6%-8% | 1%-2% | 6%-8% |
| Ex 2748 | 2%-4% | 0%-1% | 6%-8% | 1%-2% | 8%-10% |
| Ex 2749 | 2%-4% | 0%-1% | 6%-8% | 2%-4% | 0%-1% |
| Ex 2750 | 2%-4% | 0%-1% | 6%-8% | 2%-4% | 1%-2% |
| Ex 2751 | 2%-4% | 0%-1% | 6%-8% | 2%-4% | 2%-4% |
| Ex 2752 | 2%-4% | 0%-1% | 6%-8% | 2%-4% | 4%-6% |
| Ex 2753 | 2%-4% | 0%-1% | 6%-8% | 2%-4% | 6%-8% |
| Ex 2754 | 2%-4% | 0%-1% | 6%-8% | 2%-4% | 8%-10% |
| Ex 2755 | 2%-4% | 0%-1% | 6%-8% | 4%-6% | 0%-1% |
| Ex 2756 | 2%-4% | 0%-1% | 6%-8% | 4%-6% | 1%-2% |
| Ex 2757 | 2%-4% | 0%-1% | 6%-8% | 4%-6% | 2%-4% |
| Ex 2758 | 2%-4% | 0%-1% | 6%-8% | 4%-6% | 4%-6% |
| Ex 2759 | 2%-4% | 0%-1% | 6%-8% | 4%-6% | 6%-8% |
| Ex 2760 | 2%-4% | 0%-1% | 6%-8% | 4%-6% | 8%-10% |
| Ex 2761 | 2%-4% | 0%-1% | 6%-8% | 6%-8% | 0%-1% |
| Ex 2762 | 2%-4% | 0%-1% | 6%-8% | 6%-8% | 1%-2% |
| Ex 2763 | 2%-4% | 0%-1% | 6%-8% | 6%-8% | 2%-4% |
| Ex 2764 | 2%-4% | 0%-1% | 6%-8% | 6%-8% | 4%-6% |
| Ex 2765 | 2%-4% | 0%-1% | 6%-8% | 6%-8% | 6%-8% |
| Ex 2766 | 2%-4% | 0%-1% | 6%-8% | 6%-8% | 8%-10% |
| Ex 2767 | 2%-4% | 0%-1% | 6%-8% | 8%-10% | 0%-1% |
| Ex 2768 | 2%-4% | 0%-1% | 6%-8% | 8%-10% | 1%-2% |
| Ex 2769 | 2%-4% | 0%-1% | 6%-8% | 8%-10% | 2%-4% |
| Ex 2770 | 2%-4% | 0%-1% | 6%-8% | 8%-10% | 4%-6% |
| Ex 2771 | 2%-4% | 0%-1% | 6%-8% | 8%-10% | 6%-8% |
| Ex 2772 | 2%-4% | 0%-1% | 6%-8% | 8%-10% | 8%-10% |
| Ex 2773 | 2%-4% | 0%-1% | 8%-10% | 0%-1% | 0%-1% |
| Ex 2774 | 2%-4% | 0%-1% | 8%-10% | 0%-1% | 1%-2% |
| Ex 2775 | 2%-4% | 0%-1% | 8%-10% | 0%-1% | 2%-4% |
| Ex 2776 | 2%-4% | 0%-1% | 8%-10% | 0%-1% | 4%-6% |
| Ex 2777 | 2%-4% | 0%-1% | 8%-10% | 0%-1% | 6%-8% |
| Ex 2778 | 2%-4% | 0%-1% | 8%-10% | 0%-1% | 8%-10% |
| Ex 2779 | 2%-4% | 0%-1% | 8%-10% | 1%-2% | 0%-1% |
| Ex 2780 | 2%-4% | 0%-1% | 8%-10% | 1%-2% | 1%-2% |
| Ex 2781 | 2%-4% | 0%-1% | 8%-10% | 1%-2% | 2%-4% |
| Ex 2782 | 2%-4% | 0%-1% | 8%-10% | 1%-2% | 4%-6% |
| Ex 2783 | 2%-4% | 0%-1% | 8%-10% | 1%-2% | 6%-8% |
| Ex 2784 | 2%-4% | 0%-1% | 8%-10% | 1%-2% | 8%-10% |
| Ex 2785 | 2%-4% | 0%-1% | 8%-10% | 2%-4% | 0%-1% |
| Ex 2786 | 2%-4% | 0%-1% | 8%-10% | 2%-4% | 1%-2% |
| Ex 2787 | 2%-4% | 0%-1% | 8%-10% | 2%-4% | 2%-4% |
| Ex 2788 | 2%-4% | 0%-1% | 8%-10% | 2%-4% | 4%-6% |
| Ex 2789 | 2%-4% | 0%-1% | 8%-10% | 2%-4% | 6%-8% |
| Ex 2790 | 2%-4% | 0%-1% | 8%-10% | 2%-4% | 8%-10% |
| Ex 2791 | 2%-4% | 0%-1% | 8%-10% | 4%-6% | 0%-1% |
| Ex 2792 | 2%-4% | 0%-1% | 8%-10% | 4%-6% | 1%-2% |
| Ex 2793 | 2%-4% | 0%-1% | 8%-10% | 4%-6% | 2%-4% |
| Ex 2794 | 2%-4% | 0%-1% | 8%-10% | 4%-6% | 4%-6% |
| Ex 2795 | 2%-4% | 0%-1% | 8%-10% | 4%-6% | 6%-8% |
| Ex 2796 | 2%-4% | 0%-1% | 8%-10% | 4%-6% | 8%-10% |
| Ex 2797 | 2%-4% | 0%-1% | 8%-10% | 6%-8% | 0%-1% |
| Ex 2798 | 2%-4% | 0%-1% | 8%-10% | 6%-8% | 1%-2% |
| Ex 2799 | 2%-4% | 0%-1% | 8%-10% | 6%-8% | 2%-4% |
| Ex 2800 | 2%-4% | 0%-1% | 8%-10% | 6%-8% | 4%-6% |
| Ex 2801 | 2%-4% | 0%-1% | 8%-10% | 6%-8% | 6%-8% |
| Ex 2802 | 2%-4% | 0%-1% | 8%-10% | 6%-8% | 8%-10% |
| Ex 2803 | 2%-4% | 0%-1% | 8%-10% | 8%-10% | 0%-1% |
| Ex 2804 | 2%-4% | 0%-1% | 8%-10% | 8%-10% | 1%-2% |
| Ex 2805 | 2%-4% | 0%-1% | 8%-10% | 8%-10% | 2%-4% |
| Ex 2806 | 2%-4% | 0%-1% | 8%-10% | 8%-10% | 4%-6% |
| Ex 2807 | 2%-4% | 0%-1% | 8%-10% | 8%-10% | 6%-8% |
| Ex 2808 | 2%-4% | 0%-1% | 8%-10% | 8%-10% | 8%-10% |
| Ex 2809 | 2%-4% | 1%-2% | 0%-1% | 0%-1% | 0%-1% |
| Ex 2810 | 2%-4% | 1%-2% | 0%-1% | 0%-1% | 1%-2% |
| Ex 2811 | 2%-4% | 1%-2% | 0%-1% | 0%-1% | 2%-4% |
| Ex 2812 | 2%-4% | 1%-2% | 0%-1% | 0%-1% | 4%-6% |
| Ex 2813 | 2%-4% | 1%-2% | 0%-1% | 0%-1% | 6%-8% |
| Ex 2814 | 2%-4% | 1%-2% | 0%-1% | 0%-1% | 8%-10% |
| Ex 2815 | 2%-4% | 1%-2% | 0%-1% | 1%-2% | 0%-1% |
| Ex 2816 | 2%-4% | 1%-2% | 0%-1% | 1%-2% | 1%-2% |
| Ex 2817 | 2%-4% | 1%-2% | 0%-1% | 1%-2% | 2%-4% |
| Ex 2818 | 2%-4% | 1%-2% | 0%-1% | 1%-2% | 4%-6% |
| Ex 2819 | 2%-4% | 1%-2% | 0%-1% | 1%-2% | 6%-8% |
| Ex 2820 | 2%-4% | 1%-2% | 0%-1% | 1%-2% | 8%-10% |
| Ex 2821 | 2%-4% | 1%-2% | 0%-1% | 2%-4% | 0%-1% |
| Ex 2822 | 2%-4% | 1%-2% | 0%-1% | 2%-4% | 1%-2% |
| Ex 2823 | 2%-4% | 1%-2% | 0%-1% | 2%-4% | 2%-4% |
| Ex 2824 | 2%-4% | 1%-2% | 0%-1% | 2%-4% | 4%-6% |
| Ex 2825 | 2%-4% | 1%-2% | 0%-1% | 2%-4% | 6%-8% |
| Ex 2826 | 2%-4% | 1%-2% | 0%-1% | 2%-4% | 8%-10% |
| Ex 2827 | 2%-4% | 1%-2% | 0%-1% | 4%-6% | 0%-1% |
| Ex 2828 | 2%-4% | 1%-2% | 0%-1% | 4%-6% | 1%-2% |
| Ex 2829 | 2%-4% | 1%-2% | 0%-1% | 4%-6% | 2%-4% |
| Ex 2830 | 2%-4% | 1%-2% | 0%-1% | 4%-6% | 4%-6% |
| Ex 2831 | 2%-4% | 1%-2% | 0%-1% | 4%-6% | 6%-8% |
| Ex 2832 | 2%-4% | 1%-2% | 0%-1% | 4%-6% | 8%-10% |
| Ex 2833 | 2%-4% | 1%-2% | 0%-1% | 6%-8% | 0%-1% |
| Ex 2834 | 2%-4% | 1%-2% | 0%-1% | 6%-8% | 1%-2% |
| Ex 2835 | 2%-4% | 1%-2% | 0%-1% | 6%-8% | 2%-4% |
| Ex 2836 | 2%-4% | 1%-2% | 0%-1% | 6%-8% | 4%-6% |
| Ex 2837 | 2%-4% | 1%-2% | 0%-1% | 6%-8% | 6%-8% |
| Ex 2838 | 2%-4% | 1%-2% | 0%-1% | 6%-8% | 8%-10% |
| Ex 2839 | 2%-4% | 1%-2% | 0%-1% | 8%-10% | 0%-1% |
| Ex 2840 | 2%-4% | 1%-2% | 0%-1% | 8%-10% | 1%-2% |
| Ex 2841 | 2%-4% | 1%-2% | 0%-1% | 8%-10% | 2%-4% |
| Ex 2842 | 2%-4% | 1%-2% | 0%-1% | 8%-10% | 4%-6% |
| Ex 2843 | 2%-4% | 1%-2% | 0%-1% | 8%-10% | 6%-8% |
| Ex 2844 | 2%-4% | 1%-2% | 0%-1% | 8%-10% | 8%-10% |
| Ex 2845 | 2%-4% | 1%-2% | 1%-2% | 0%-1% | 0%-1% |
| Ex 2846 | 2%-4% | 1%-2% | 1%-2% | 0%-1% | 1%-2% |
| Ex 2847 | 2%-4% | 1%-2% | 1%-2% | 0%-1% | 2%-4% |
| Ex 2848 | 2%-4% | 1%-2% | 1%-2% | 0%-1% | 4%-6% |
| Ex 2849 | 2%-4% | 1%-2% | 1%-2% | 0%-1% | 6%-8% |
| Ex 2850 | 2%-4% | 1%-2% | 1%-2% | 0%-1% | 8%-10% |
| Ex 2851 | 2%-4% | 1%-2% | 1%-2% | 1%-2% | 0%-1% |
| Ex 2852 | 2%-4% | 1%-2% | 1%-2% | 1%-2% | 1%-2% |
| Ex 2853 | 2%-4% | 1%-2% | 1%-2% | 1%-2% | 2%-4% |
| Ex 2854 | 2%-4% | 1%-2% | 1%-2% | 1%-2% | 4%-6% |
| Ex 2855 | 2%-4% | 1%-2% | 1%-2% | 1%-2% | 6%-8% |
| Ex 2856 | 2%-4% | 1%-2% | 1%-2% | 1%-2% | 8%-10% |
| Ex 2857 | 2%-4% | 1%-2% | 1%-2% | 2%-4% | 0%-1% |
| Ex 2858 | 2%-4% | 1%-2% | 1%-2% | 2%-4% | 1%-2% |
| Ex 2859 | 2%-4% | 1%-2% | 1%-2% | 2%-4% | 2%-4% |
| Ex 2860 | 2%-4% | 1%-2% | 1%-2% | 2%-4% | 4%-6% |
| Ex 2861 | 2%-4% | 1%-2% | 1%-2% | 2%-4% | 6%-8% |
| Ex 2862 | 2%-4% | 1%-2% | 1%-2% | 2%-4% | 8%-10% |
| Ex 2863 | 2%-4% | 1%-2% | 1%-2% | 4%-6% | 0%-1% |
| Ex 2864 | 2%-4% | 1%-2% | 1%-2% | 4%-6% | 1%-2% |
| Ex 2865 | 2%-4% | 1%-2% | 1%-2% | 4%-6% | 2%-4% |
| Ex 2866 | 2%-4% | 1%-2% | 1%-2% | 4%-6% | 4%-6% |
| Ex 2867 | 2%-4% | 1%-2% | 1%-2% | 4%-6% | 6%-8% |
| Ex 2868 | 2%-4% | 1%-2% | 1%-2% | 4%-6% | 8%-10% |
| Ex 2869 | 2%-4% | 1%-2% | 1%-2% | 6%-8% | 0%-1% |
| Ex 2870 | 2%-4% | 1%-2% | 1%-2% | 6%-8% | 1%-2% |
| Ex 2871 | 2%-4% | 1%-2% | 1%-2% | 6%-8% | 2%-4% |
| Ex 2872 | 2%-4% | 1%-2% | 1%-2% | 6%-8% | 4%-6% |
| Ex 2873 | 2%-4% | 1%-2% | 1%-2% | 6%-8% | 6%-8% |
| Ex 2874 | 2%-4% | 1%-2% | 1%-2% | 6%-8% | 8%-10% |
| Ex 2875 | 2%-4% | 1%-2% | 1%-2% | 8%-10% | 0%-1% |
| Ex 2876 | 2%-4% | 1%-2% | 1%-2% | 8%-10% | 1%-2% |
| Ex 2877 | 2%-4% | 1%-2% | 1%-2% | 8%-10% | 2%-4% |
| Ex 2878 | 2%-4% | 1%-2% | 1%-2% | 8%-10% | 4%-6% |
| Ex 2879 | 2%-4% | 1%-2% | 1%-2% | 8%-10% | 6%-8% |
| Ex 2880 | 2%-4% | 1%-2% | 1%-2% | 8%-10% | 8%-10% |
| Ex 2881 | 2%-4% | 1%-2% | 2%-4% | 0%-1% | 0%-1% |
| Ex 2882 | 2%-4% | 1%-2% | 2%-4% | 0%-1% | 1%-2% |
| Ex 2883 | 2%-4% | 1%-2% | 2%-4% | 0%-1% | 2%-4% |
| Ex 2884 | 2%-4% | 1%-2% | 2%-4% | 0%-1% | 4%-6% |
| Ex 2885 | 2%-4% | 1%-2% | 2%-4% | 0%-1% | 6%-8% |
| Ex 2886 | 2%-4% | 1%-2% | 2%-4% | 0%-1% | 8%-10% |
| Ex 2887 | 2%-4% | 1%-2% | 2%-4% | 1%-2% | 0%-1% |
| Ex 2888 | 2%-4% | 1%-2% | 2%-4% | 1%-2% | 1%-2% |
| Ex 2889 | 2%-4% | 1%-2% | 2%-4% | 1%-2% | 2%-4% |
| Ex 2890 | 2%-4% | 1%-2% | 2%-4% | 1%-2% | 4%-6% |
| Ex 2891 | 2%-4% | 1%-2% | 2%-4% | 1%-2% | 6%-8% |
| Ex 2892 | 2%-4% | 1%-2% | 2%-4% | 1%-2% | 8%-10% |
| Ex 2893 | 2%-4% | 1%-2% | 2%-4% | 2%-4% | 0%-1% |
| Ex 2894 | 2%-4% | 1%-2% | 2%-4% | 2%-4% | 1%-2% |
| Ex 2895 | 2%-4% | 1%-2% | 2%-4% | 2%-4% | 2%-4% |
| Ex 2896 | 2%-4% | 1%-2% | 2%-4% | 2%-4% | 4%-6% |
| Ex 2897 | 2%-4% | 1%-2% | 2%-4% | 2%-4% | 6%-8% |
| Ex 2898 | 2%-4% | 1%-2% | 2%-4% | 2%-4% | 8%-10% |
| Ex 2899 | 2%-4% | 1%-2% | 2%-4% | 4%-6% | 0%-1% |
| Ex 2900 | 2%-4% | 1%-2% | 2%-4% | 4%-6% | 1%-2% |
| Ex 2901 | 2%-4% | 1%-2% | 2%-4% | 4%-6% | 2%-4% |
| Ex 2902 | 2%-4% | 1%-2% | 2%-4% | 4%-6% | 4%-6% |
| Ex 2903 | 2%-4% | 1%-2% | 2%-4% | 4%-6% | 6%-8% |
| Ex 2904 | 2%-4% | 1%-2% | 2%-4% | 4%-6% | 8%-10% |
| Ex 2905 | 2%-4% | 1%-2% | 2%-4% | 6%-8% | 0%-1% |
| Ex 2906 | 2%-4% | 1%-2% | 2%-4% | 6%-8% | 1%-2% |
| Ex 2907 | 2%-4% | 1%-2% | 2%-4% | 6%-8% | 2%-4% |
| Ex 2908 | 2%-4% | 1%-2% | 2%-4% | 6%-8% | 4%-6% |
| Ex 2909 | 2%-4% | 1%-2% | 2%-4% | 6%-8% | 6%-8% |
| Ex 2910 | 2%-4% | 1%-2% | 2%-4% | 6%-8% | 8%-10% |
| Ex 2911 | 2%-4% | 1%-2% | 2%-4% | 8%-10% | 0%-1% |
| Ex 2912 | 2%-4% | 1%-2% | 2%-4% | 8%-10% | 1%-2% |
| Ex 2913 | 2%-4% | 1%-2% | 2%-4% | 8%-10% | 2%-4% |
| Ex 2914 | 2%-4% | 1%-2% | 2%-4% | 8%-10% | 4%-6% |
| Ex 2915 | 2%-4% | 1%-2% | 2%-4% | 8%-10% | 6%-8% |
| Ex 2916 | 2%-4% | 1%-2% | 2%-4% | 8%-10% | 8%-10% |
| Ex 2917 | 2%-4% | 1%-2% | 4%-6% | 0%-1% | 0%-1% |
| Ex 2918 | 2%-4% | 1%-2% | 4%-6% | 0%-1% | 1%-2% |
| Ex 2919 | 2%-4% | 1%-2% | 4%-6% | 0%-1% | 2%-4% |
| Ex 2920 | 2%-4% | 1%-2% | 4%-6% | 0%-1% | 4%-6% |
| Ex 2921 | 2%-4% | 1%-2% | 4%-6% | 0%-1% | 6%-8% |
| Ex 2922 | 2%-4% | 1%-2% | 4%-6% | 0%-1% | 8%-10% |
| Ex 2923 | 2%-4% | 1%-2% | 4%-6% | 1%-2% | 0%-1% |
| Ex 2924 | 2%-4% | 1%-2% | 4%-6% | 1%-2% | 1%-2% |
| Ex 2925 | 2%-4% | 1%-2% | 4%-6% | 1%-2% | 2%-4% |
| Ex 2926 | 2%-4% | 1%-2% | 4%-6% | 1%-2% | 4%-6% |
| Ex 2927 | 2%-4% | 1%-2% | 4%-6% | 1%-2% | 6%-8% |
| Ex 2928 | 2%-4% | 1%-2% | 4%-6% | 1%-2% | 8%-10% |
| Ex 2929 | 2%-4% | 1%-2% | 4%-6% | 2%-4% | 0%-1% |
| Ex 2930 | 2%-4% | 1%-2% | 4%-6% | 2%-4% | 1%-2% |
| Ex 2931 | 2%-4% | 1%-2% | 4%-6% | 2%-4% | 2%-4% |
| Ex 2932 | 2%-4% | 1%-2% | 4%-6% | 2%-4% | 4%-6% |
| Ex 2933 | 2%-4% | 1%-2% | 4%-6% | 2%-4% | 6%-8% |
| Ex 2934 | 2%-4% | 1%-2% | 4%-6% | 2%-4% | 8%-10% |
| Ex 2935 | 2%-4% | 1%-2% | 4%-6% | 4%-6% | 0%-1% |
| Ex 2936 | 2%-4% | 1%-2% | 4%-6% | 4%-6% | 1%-2% |
| Ex 2937 | 2%-4% | 1%-2% | 4%-6% | 4%-6% | 2%-4% |
| Ex 2938 | 2%-4% | 1%-2% | 4%-6% | 4%-6% | 4%-6% |
| Ex 2939 | 2%-4% | 1%-2% | 4%-6% | 4%-6% | 6%-8% |
| Ex 2940 | 2%-4% | 1%-2% | 4%-6% | 4%-6% | 8%-10% |
| Ex 2941 | 2%-4% | 1%-2% | 4%-6% | 6%-8% | 0%-1% |
| Ex 2942 | 2%-4% | 1%-2% | 4%-6% | 6%-8% | 1%-2% |
| Ex 2943 | 2%-4% | 1%-2% | 4%-6% | 6%-8% | 2%-4% |
| Ex 2944 | 2%-4% | 1%-2% | 4%-6% | 6%-8% | 4%-6% |
| Ex 2945 | 2%-4% | 1%-2% | 4%-6% | 6%-8% | 6%-8% |
| Ex 2946 | 2%-4% | 1%-2% | 4%-6% | 6%-8% | 8%-10% |
| Ex 2947 | 2%-4% | 1%-2% | 4%-6% | 8%-10% | 0%-1% |
| Ex 2948 | 2%-4% | 1%-2% | 4%-6% | 8%-10% | 1%-2% |
| Ex 2949 | 2%-4% | 1%-2% | 4%-6% | 8%-10% | 2%-4% |
| Ex 2950 | 2%-4% | 1%-2% | 4%-6% | 8%-10% | 4%-6% |
| Ex 2951 | 2%-4% | 1%-2% | 4%-6% | 8%-10% | 6%-8% |
| Ex 2952 | 2%-4% | 1%-2% | 4%-6% | 8%-10% | 8%-10% |
| Ex 2953 | 2%-4% | 1%-2% | 6%-8% | 0%-1% | 0%-1% |
| Ex 2954 | 2%-4% | 1%-2% | 6%-8% | 0%-1% | 1%-2% |
| Ex 2955 | 2%-4% | 1%-2% | 6%-8% | 0%-1% | 2%-4% |
| Ex 2956 | 2%-4% | 1%-2% | 6%-8% | 0%-1% | 4%-6% |
| Ex 2957 | 2%-4% | 1%-2% | 6%-8% | 0%-1% | 6%-8% |
| Ex 2958 | 2%-4% | 1%-2% | 6%-8% | 0%-1% | 8%-10% |
| Ex 2959 | 2%-4% | 1%-2% | 6%-8% | 1%-2% | 0%-1% |
| Ex 2960 | 2%-4% | 1%-2% | 6%-8% | 1%-2% | 1%-2% |
| Ex 2961 | 2%-4% | 1%-2% | 6%-8% | 1%-2% | 2%-4% |
| Ex 2962 | 2%-4% | 1%-2% | 6%-8% | 1%-2% | 4%-6% |
| Ex 2963 | 2%-4% | 1%-2% | 6%-8% | 1%-2% | 6%-8% |
| Ex 2964 | 2%-4% | 1%-2% | 6%-8% | 1%-2% | 8%-10% |
| Ex 2965 | 2%-4% | 1%-2% | 6%-8% | 2%-4% | 0%-1% |
| Ex 2966 | 2%-4% | 1%-2% | 6%-8% | 2%-4% | 1%-2% |
| Ex 2967 | 2%-4% | 1%-2% | 6%-8% | 2%-4% | 2%-4% |
| Ex 2968 | 2%-4% | 1%-2% | 6%-8% | 2%-4% | 4%-6% |
| Ex 2969 | 2%-4% | 1%-2% | 6%-8% | 2%-4% | 6%-8% |
| Ex 2970 | 2%-4% | 1%-2% | 6%-8% | 2%-4% | 8%-10% |
| Ex 2971 | 2%-4% | 1%-2% | 6%-8% | 4%-6% | 0%-1% |
| Ex 2972 | 2%-4% | 1%-2% | 6%-8% | 4%-6% | 1%-2% |
| Ex 2973 | 2%-4% | 1%-2% | 6%-8% | 4%-6% | 2%-4% |
| Ex 2974 | 2%-4% | 1%-2% | 6%-8% | 4%-6% | 4%-6% |
| Ex 2975 | 2%-4% | 1%-2% | 6%-8% | 4%-6% | 6%-8% |
| Ex 2976 | 2%-4% | 1%-2% | 6%-8% | 4%-6% | 8%-10% |
| Ex 2977 | 2%-4% | 1%-2% | 6%-8% | 6%-8% | 0%-1% |
| Ex 2978 | 2%-4% | 1%-2% | 6%-8% | 6%-8% | 1%-2% |
| Ex 2979 | 2%-4% | 1%-2% | 6%-8% | 6%-8% | 2%-4% |
| Ex 2980 | 2%-4% | 1%-2% | 6%-8% | 6%-8% | 4%-6% |
| Ex 2981 | 2%-4% | 1%-2% | 6%-8% | 6%-8% | 6%-8% |
| Ex 2982 | 2%-4% | 1%-2% | 6%-8% | 6%-8% | 8%-10% |
| Ex 2983 | 2%-4% | 1%-2% | 6%-8% | 8%-10% | 0%-1% |
| Ex 2984 | 2%-4% | 1%-2% | 6%-8% | 8%-10% | 1%-2% |
| Ex 2985 | 2%-4% | 1%-2% | 6%-8% | 8%-10% | 2%-4% |
| Ex 2986 | 2%-4% | 1%-2% | 6%-8% | 8%-10% | 4%-6% |
| Ex 2987 | 2%-4% | 1%-2% | 6%-8% | 8%-10% | 6%-8% |
| Ex 2988 | 2%-4% | 1%-2% | 6%-8% | 8%-10% | 8%-10% |
| Ex 2989 | 2%-4% | 1%-2% | 8%-10% | 0%-1% | 0%-1% |
| Ex 2990 | 2%-4% | 1%-2% | 8%-10% | 0%-1% | 1%-2% |
| Ex 2991 | 2%-4% | 1%-2% | 8%-10% | 0%-1% | 2%-4% |
| Ex 2992 | 2%-4% | 1%-2% | 8%-10% | 0%-1% | 4%-6% |
| Ex 2993 | 2%-4% | 1%-2% | 8%-10% | 0%-1% | 6%-8% |
| Ex 2994 | 2%-4% | 1%-2% | 8%-10% | 0%-1% | 8%-10% |
| Ex 2995 | 2%-4% | 1%-2% | 8%-10% | 1%-2% | 0%-1% |
| Ex 2996 | 2%-4% | 1%-2% | 8%-10% | 1%-2% | 1%-2% |
| Ex 2997 | 2%-4% | 1%-2% | 8%-10% | 1%-2% | 2%-4% |
| Ex 2998 | 2%-4% | 1%-2% | 8%-10% | 1%-2% | 4%-6% |
| Ex 2999 | 2%-4% | 1%-2% | 8%-10% | 1%-2% | 6%-8% |
| Ex 3000 | 2%-4% | 1%-2% | 8%-10% | 1%-2% | 8%-10% |
| Ex 3001 | 2%-4% | 1%-2% | 8%-10% | 2%-4% | 0%-1% |
| Ex 3002 | 2%-4% | 1%-2% | 8%-10% | 2%-4% | 1%-2% |
| Ex 3003 | 2%-4% | 1%-2% | 8%-10% | 2%-4% | 2%-4% |
| Ex 3004 | 2%-4% | 1%-2% | 8%-10% | 2%-4% | 4%-6% |
| Ex 3005 | 2%-4% | 1%-2% | 8%-10% | 2%-4% | 6%-8% |
| Ex 3006 | 2%-4% | 1%-2% | 8%-10% | 2%-4% | 8%-10% |
| Ex 3007 | 2%-4% | 1%-2% | 8%-10% | 4%-6% | 0%-1% |
| Ex 3008 | 2%-4% | 1%-2% | 8%-10% | 4%-6% | 1%-2% |
| Ex 3009 | 2%-4% | 1%-2% | 8%-10% | 4%-6% | 2%-4% |
| Ex 3010 | 2%-4% | 1%-2% | 8%-10% | 4%-6% | 4%-6% |
| Ex 3011 | 2%-4% | 1%-2% | 8%-10% | 4%-6% | 6%-8% |
| Ex 3012 | 2%-4% | 1%-2% | 8%-10% | 4%-6% | 8%-10% |
| Ex 3013 | 2%-4% | 1%-2% | 8%-10% | 6%-8% | 0%-1% |
| Ex 3014 | 2%-4% | 1%-2% | 8%-10% | 6%-8% | 1%-2% |
| Ex 3015 | 2%-4% | 1%-2% | 8%-10% | 6%-8% | 2%-4% |
| Ex 3016 | 2%-4% | 1%-2% | 8%-10% | 6%-8% | 4%-6% |
| Ex 3017 | 2%-4% | 1%-2% | 8%-10% | 6%-8% | 6%-8% |
| Ex 3018 | 2%-4% | 1%-2% | 8%-10% | 6%-8% | 8%-10% |
| Ex 3019 | 2%-4% | 1%-2% | 8%-10% | 8%-10% | 0%-1% |
| Ex 3020 | 2%-4% | 1%-2% | 8%-10% | 8%-10% | 1%-2% |
| Ex 3021 | 2%-4% | 1%-2% | 8%-10% | 8%-10% | 2%-4% |
| Ex 3022 | 2%-4% | 1%-2% | 8%-10% | 8%-10% | 4%-6% |
| Ex 3023 | 2%-4% | 1%-2% | 8%-10% | 8%-10% | 6%-8% |
| Ex 3024 | 2%-4% | 1%-2% | 8%-10% | 8%-10% | 8%-10% |
| Ex 3025 | 2%-4% | 2%-4% | 0%-1% | 0%-1% | 0%-1% |
| Ex 3026 | 2%-4% | 2%-4% | 0%-1% | 0%-1% | 1%-2% |
| Ex 3027 | 2%-4% | 2%-4% | 0%-1% | 0%-1% | 2%-4% |
| Ex 3028 | 2%-4% | 2%-4% | 0%-1% | 0%-1% | 4%-6% |
| Ex 3029 | 2%-4% | 2%-4% | 0%-1% | 0%-1% | 6%-8% |
| Ex 3030 | 2%-4% | 2%-4% | 0%-1% | 0%-1% | 8%-10% |
| Ex 3031 | 2%-4% | 2%-4% | 0%-1% | 1%-2% | 0%-1% |
| Ex 3032 | 2%-4% | 2%-4% | 0%-1% | 1%-2% | 1%-2% |
| Ex 3033 | 2%-4% | 2%-4% | 0%-1% | 1%-2% | 2%-4% |
| Ex 3034 | 2%-4% | 2%-4% | 0%-1% | 1%-2% | 4%-6% |
| Ex 3035 | 2%-4% | 2%-4% | 0%-1% | 1%-2% | 6%-8% |
| Ex 3036 | 2%-4% | 2%-4% | 0%-1% | 1%-2% | 8%-10% |
| Ex 3037 | 2%-4% | 2%-4% | 0%-1% | 2%-4% | 0%-1% |
| Ex 3038 | 2%-4% | 2%-4% | 0%-1% | 2%-4% | 1%-2% |
| Ex 3039 | 2%-4% | 2%-4% | 0%-1% | 2%-4% | 2%-4% |
| Ex 3040 | 2%-4% | 2%-4% | 0%-1% | 2%-4% | 4%-6% |
| Ex 3041 | 2%-4% | 2%-4% | 0%-1% | 2%-4% | 6%-8% |
| Ex 3042 | 2%-4% | 2%-4% | 0%-1% | 2%-4% | 8%-10% |
| Ex 3043 | 2%-4% | 2%-4% | 0%-1% | 4%-6% | 0%-1% |
| Ex 3044 | 2%-4% | 2%-4% | 0%-1% | 4%-6% | 1%-2% |
| Ex 3045 | 2%-4% | 2%-4% | 0%-1% | 4%-6% | 2%-4% |
| Ex 3046 | 2%-4% | 2%-4% | 0%-1% | 4%-6% | 4%-6% |
| Ex 3047 | 2%-4% | 2%-4% | 0%-1% | 4%-6% | 6%-8% |
| Ex 3048 | 2%-4% | 2%-4% | 0%-1% | 4%-6% | 8%-10% |
| Ex 3049 | 2%-4% | 2%-4% | 0%-1% | 6%-8% | 0%-1% |
| Ex 3050 | 2%-4% | 2%-4% | 0%-1% | 6%-8% | 1%-2% |
| Ex 3051 | 2%-4% | 2%-4% | 0%-1% | 6%-8% | 2%-4% |
| Ex 3052 | 2%-4% | 2%-4% | 0%-1% | 6%-8% | 4%-6% |
| Ex 3053 | 2%-4% | 2%-4% | 0%-1% | 6%-8% | 6%-8% |
| Ex 3054 | 2%-4% | 2%-4% | 0%-1% | 6%-8% | 8%-10% |
| Ex 3055 | 2%-4% | 2%-4% | 0%-1% | 8%-10% | 0%-1% |
| Ex 3056 | 2%-4% | 2%-4% | 0%-1% | 8%-10% | 1%-2% |
| Ex 3057 | 2%-4% | 2%-4% | 0%-1% | 8%-10% | 2%-4% |
| Ex 3058 | 2%-4% | 2%-4% | 0%-1% | 8%-10% | 4%-6% |
| Ex 3059 | 2%-4% | 2%-4% | 0%-1% | 8%-10% | 6%-8% |
| Ex 3060 | 2%-4% | 2%-4% | 0%-1% | 8%-10% | 8%-10% |
| Ex 3061 | 2%-4% | 2%-4% | 1%-2% | 0%-1% | 0%-1% |
| Ex 3062 | 2%-4% | 2%-4% | 1%-2% | 0%-1% | 1%-2% |
| Ex 3063 | 2%-4% | 2%-4% | 1%-2% | 0%-1% | 2%-4% |
| Ex 3064 | 2%-4% | 2%-4% | 1%-2% | 0%-1% | 4%-6% |
| Ex 3065 | 2%-4% | 2%-4% | 1%-2% | 0%-1% | 6%-8% |
| Ex 3066 | 2%-4% | 2%-4% | 1%-2% | 0%-1% | 8%-10% |
| Ex 3067 | 2%-4% | 2%-4% | 1%-2% | 1%-2% | 0%-1% |
| Ex 3068 | 2%-4% | 2%-4% | 1%-2% | 1%-2% | 1%-2% |
| Ex 3069 | 2%-4% | 2%-4% | 1%-2% | 1%-2% | 2%-4% |
| Ex 3070 | 2%-4% | 2%-4% | 1%-2% | 1%-2% | 4%-6% |
| Ex 3071 | 2%-4% | 2%-4% | 1%-2% | 1%-2% | 6%-8% |
| Ex 3072 | 2%-4% | 2%-4% | 1%-2% | 1%-2% | 8%-10% |
| Ex 3073 | 2%-4% | 2%-4% | 1%-2% | 2%-4% | 0%-1% |
| Ex 3074 | 2%-4% | 2%-4% | 1%-2% | 2%-4% | 1%-2% |
| Ex 3075 | 2%-4% | 2%-4% | 1%-2% | 2%-4% | 2%-4% |
| Ex 3076 | 2%-4% | 2%-4% | 1%-2% | 2%-4% | 4%-6% |
| Ex 3077 | 2%-4% | 2%-4% | 1%-2% | 2%-4% | 6%-8% |
| Ex 3078 | 2%-4% | 2%-4% | 1%-2% | 2%-4% | 8%-10% |
| Ex 3079 | 2%-4% | 2%-4% | 1%-2% | 4%-6% | 0%-1% |
| Ex 3080 | 2%-4% | 2%-4% | 1%-2% | 4%-6% | 1%-2% |
| Ex 3081 | 2%-4% | 2%-4% | 1%-2% | 4%-6% | 2%-4% |
| Ex 3082 | 2%-4% | 2%-4% | 1%-2% | 4%-6% | 4%-6% |
| Ex 3083 | 2%-4% | 2%-4% | 1%-2% | 4%-6% | 6%-8% |
| Ex 3084 | 2%-4% | 2%-4% | 1%-2% | 4%-6% | 8%-10% |
| Ex 3085 | 2%-4% | 2%-4% | 1%-2% | 6%-8% | 0%-1% |
| Ex 3086 | 2%-4% | 2%-4% | 1%-2% | 6%-8% | 1%-2% |
| Ex 3087 | 2%-4% | 2%-4% | 1%-2% | 6%-8% | 2%-4% |
| Ex 3088 | 2%-4% | 2%-4% | 1%-2% | 6%-8% | 4%-6% |
| Ex 3089 | 2%-4% | 2%-4% | 1%-2% | 6%-8% | 6%-8% |
| Ex 3090 | 2%-4% | 2%-4% | 1%-2% | 6%-8% | 8%-10% |
| Ex 3091 | 2%-4% | 2%-4% | 1%-2% | 8%-10% | 0%-1% |
| Ex 3092 | 2%-4% | 2%-4% | 1%-2% | 8%-10% | 1%-2% |
| Ex 3093 | 2%-4% | 2%-4% | 1%-2% | 8%-10% | 2%-4% |
| Ex 3094 | 2%-4% | 2%-4% | 1%-2% | 8%-10% | 4%-6% |
| Ex 3095 | 2%-4% | 2%-4% | 1%-2% | 8%-10% | 6%-8% |
| Ex 3096 | 2%-4% | 2%-4% | 1%-2% | 8%-10% | 8%-10% |
| Ex 3097 | 2%-4% | 2%-4% | 2%-4% | 0%-1% | 0%-1% |
| Ex 3098 | 2%-4% | 2%-4% | 2%-4% | 0%-1% | 1%-2% |
| Ex 3099 | 2%-4% | 2%-4% | 2%-4% | 0%-1% | 2%-4% |
| Ex 3100 | 2%-4% | 2%-4% | 2%-4% | 0%-1% | 4%-6% |
| Ex 3101 | 2%-4% | 2%-4% | 2%-4% | 0%-1% | 6%-8% |
| Ex 3102 | 2%-4% | 2%-4% | 2%-4% | 0%-1% | 8%-10% |
| Ex 3103 | 2%-4% | 2%-4% | 2%-4% | 1%-2% | 0%-1% |
| Ex 3104 | 2%-4% | 2%-4% | 2%-4% | 1%-2% | 1%-2% |
| Ex 3105 | 2%-4% | 2%-4% | 2%-4% | 1%-2% | 2%-4% |
| Ex 3106 | 2%-4% | 2%-4% | 2%-4% | 1%-2% | 4%-6% |
| Ex 3107 | 2%-4% | 2%-4% | 2%-4% | 1%-2% | 6%-8% |
| Ex 3108 | 2%-4% | 2%-4% | 2%-4% | 1%-2% | 8%-10% |
| Ex 3109 | 2%-4% | 2%-4% | 2%-4% | 2%-4% | 0%-1% |
| Ex 3110 | 2%-4% | 2%-4% | 2%-4% | 2%-4% | 1%-2% |
| Ex 3111 | 2%-4% | 2%-4% | 2%-4% | 2%-4% | 2%-4% |
| Ex 3112 | 2%-4% | 2%-4% | 2%-4% | 2%-4% | 4%-6% |
| Ex 3113 | 2%-4% | 2%-4% | 2%-4% | 2%-4% | 6%-8% |
| Ex 3114 | 2%-4% | 2%-4% | 2%-4% | 2%-4% | 8%-10% |
| Ex 3115 | 2%-4% | 2%-4% | 2%-4% | 4%-6% | 0%-1% |
| Ex 3116 | 2%-4% | 2%-4% | 2%-4% | 4%-6% | 1%-2% |
| Ex 3117 | 2%-4% | 2%-4% | 2%-4% | 4%-6% | 2%-4% |
| Ex 3118 | 2%-4% | 2%-4% | 2%-4% | 4%-6% | 4%-6% |
| Ex 3119 | 2%-4% | 2%-4% | 2%-4% | 4%-6% | 6%-8% |
| Ex 3120 | 2%-4% | 2%-4% | 2%-4% | 4%-6% | 8%-10% |
| Ex 3121 | 2%-4% | 2%-4% | 2%-4% | 6%-8% | 0%-1% |
| Ex 3122 | 2%-4% | 2%-4% | 2%-4% | 6%-8% | 1%-2% |
| Ex 3123 | 2%-4% | 2%-4% | 2%-4% | 6%-8% | 2%-4% |
| Ex 3124 | 2%-4% | 2%-4% | 2%-4% | 6%-8% | 4%-6% |
| Ex 3125 | 2%-4% | 2%-4% | 2%-4% | 6%-8% | 6%-8% |
| Ex 3126 | 2%-4% | 2%-4% | 2%-4% | 6%-8% | 8%-10% |
| Ex 3127 | 2%-4% | 2%-4% | 2%-4% | 8%-10% | 0%-1% |
| Ex 3128 | 2%-4% | 2%-4% | 2%-4% | 8%-10% | 1%-2% |
| Ex 3129 | 2%-4% | 2%-4% | 2%-4% | 8%-10% | 2%-4% |
| Ex 3130 | 2%-4% | 2%-4% | 2%-4% | 8%-10% | 4%-6% |
| Ex 3131 | 2%-4% | 2%-4% | 2%-4% | 8%-10% | 6%-8% |
| Ex 3132 | 2%-4% | 2%-4% | 2%-4% | 8%-10% | 8%-10% |
| Ex 3133 | 2%-4% | 2%-4% | 4%-6% | 0%-1% | 0%-1% |
| Ex 3134 | 2%-4% | 2%-4% | 4%-6% | 0%-1% | 1%-2% |
| Ex 3135 | 2%-4% | 2%-4% | 4%-6% | 0%-1% | 2%-4% |
| Ex 3136 | 2%-4% | 2%-4% | 4%-6% | 0%-1% | 4%-6% |
| Ex 3137 | 2%-4% | 2%-4% | 4%-6% | 0%-1% | 6%-8% |
| Ex 3138 | 2%-4% | 2%-4% | 4%-6% | 0%-1% | 8%-10% |
| Ex 3139 | 2%-4% | 2%-4% | 4%-6% | 1%-2% | 0%-1% |
| Ex 3140 | 2%-4% | 2%-4% | 4%-6% | 1%-2% | 1%-2% |
| Ex 3141 | 2%-4% | 2%-4% | 4%-6% | 1%-2% | 2%-4% |
| Ex 3142 | 2%-4% | 2%-4% | 4%-6% | 1%-2% | 4%-6% |
| Ex 3143 | 2%-4% | 2%-4% | 4%-6% | 1%-2% | 6%-8% |
| Ex 3144 | 2%-4% | 2%-4% | 4%-6% | 1%-2% | 8%-10% |
| Ex 3145 | 2%-4% | 2%-4% | 4%-6% | 2%-4% | 0%-1% |
| Ex 3146 | 2%-4% | 2%-4% | 4%-6% | 2%-4% | 1%-2% |
| Ex 3147 | 2%-4% | 2%-4% | 4%-6% | 2%-4% | 2%-4% |
| Ex 3148 | 2%-4% | 2%-4% | 4%-6% | 2%-4% | 4%-6% |
| Ex 3149 | 2%-4% | 2%-4% | 4%-6% | 2%-4% | 6%-8% |
| Ex 3150 | 2%-4% | 2%-4% | 4%-6% | 2%-4% | 8%-10% |
| Ex 3151 | 2%-4% | 2%-4% | 4%-6% | 4%-6% | 0%-1% |
| Ex 3152 | 2%-4% | 2%-4% | 4%-6% | 4%-6% | 1%-2% |
| Ex 3153 | 2%-4% | 2%-4% | 4%-6% | 4%-6% | 2%-4% |
| Ex 3154 | 2%-4% | 2%-4% | 4%-6% | 4%-6% | 4%-6% |
| Ex 3155 | 2%-4% | 2%-4% | 4%-6% | 4%-6% | 6%-8% |
| Ex 3156 | 2%-4% | 2%-4% | 4%-6% | 4%-6% | 8%-10% |
| Ex 3157 | 2%-4% | 2%-4% | 4%-6% | 6%-8% | 0%-1% |
| Ex 3158 | 2%-4% | 2%-4% | 4%-6% | 6%-8% | 1%-2% |
| Ex 3159 | 2%-4% | 2%-4% | 4%-6% | 6%-8% | 2%-4% |
| Ex 3160 | 2%-4% | 2%-4% | 4%-6% | 6%-8% | 4%-6% |
| Ex 3161 | 2%-4% | 2%-4% | 4%-6% | 6%-8% | 6%-8% |
| Ex 3162 | 2%-4% | 2%-4% | 4%-6% | 6%-8% | 8%-10% |
| Ex 3163 | 2%-4% | 2%-4% | 4%-6% | 8%-10% | 0%-1% |
| Ex 3164 | 2%-4% | 2%-4% | 4%-6% | 8%-10% | 1%-2% |
| Ex 3165 | 2%-4% | 2%-4% | 4%-6% | 8%-10% | 2%-4% |
| Ex 3166 | 2%-4% | 2%-4% | 4%-6% | 8%-10% | 4%-6% |
| Ex 3167 | 2%-4% | 2%-4% | 4%-6% | 8%-10% | 6%-8% |
| Ex 3168 | 2%-4% | 2%-4% | 4%-6% | 8%-10% | 8%-10% |
| Ex 3169 | 2%-4% | 2%-4% | 6%-8% | 0%-1% | 0%-1% |
| Ex 3170 | 2%-4% | 2%-4% | 6%-8% | 0%-1% | 1%-2% |
| Ex 3171 | 2%-4% | 2%-4% | 6%-8% | 0%-1% | 2%-4% |
| Ex 3172 | 2%-4% | 2%-4% | 6%-8% | 0%-1% | 4%-6% |
| Ex 3173 | 2%-4% | 2%-4% | 6%-8% | 0%-1% | 6%-8% |
| Ex 3174 | 2%-4% | 2%-4% | 6%-8% | 0%-1% | 8%-10% |
| Ex 3175 | 2%-4% | 2%-4% | 6%-8% | 1%-2% | 0%-1% |
| Ex 3176 | 2%-4% | 2%-4% | 6%-8% | 1%-2% | 1%-2% |
| Ex 3177 | 2%-4% | 2%-4% | 6%-8% | 1%-2% | 2%-4% |
| Ex 3178 | 2%-4% | 2%-4% | 6%-8% | 1%-2% | 4%-6% |
| Ex 3179 | 2%-4% | 2%-4% | 6%-8% | 1%-2% | 6%-8% |
| Ex 3180 | 2%-4% | 2%-4% | 6%-8% | 1%-2% | 8%-10% |
| Ex 3181 | 2%-4% | 2%-4% | 6%-8% | 2%-4% | 0%-1% |
| Ex 3182 | 2%-4% | 2%-4% | 6%-8% | 2%-4% | 1%-2% |
| Ex 3183 | 2%-4% | 2%-4% | 6%-8% | 2%-4% | 2%-4% |
| Ex 3184 | 2%-4% | 2%-4% | 6%-8% | 2%-4% | 4%-6% |
| Ex 3185 | 2%-4% | 2%-4% | 6%-8% | 2%-4% | 6%-8% |
| Ex 3186 | 2%-4% | 2%-4% | 6%-8% | 2%-4% | 8%-10% |
| Ex 3187 | 2%-4% | 2%-4% | 6%-8% | 4%-6% | 0%-1% |
| Ex 3188 | 2%-4% | 2%-4% | 6%-8% | 4%-6% | 1%-2% |
| Ex 3189 | 2%-4% | 2%-4% | 6%-8% | 4%-6% | 2%-4% |
| Ex 3190 | 2%-4% | 2%-4% | 6%-8% | 4%-6% | 4%-6% |
| Ex 3191 | 2%-4% | 2%-4% | 6%-8% | 4%-6% | 6%-8% |
| Ex 3192 | 2%-4% | 2%-4% | 6%-8% | 4%-6% | 8%-10% |
| Ex 3193 | 2%-4% | 2%-4% | 6%-8% | 6%-8% | 0%-1% |
| Ex 3194 | 2%-4% | 2%-4% | 6%-8% | 6%-8% | 1%-2% |
| Ex 3195 | 2%-4% | 2%-4% | 6%-8% | 6%-8% | 2%-4% |
| Ex 3196 | 2%-4% | 2%-4% | 6%-8% | 6%-8% | 4%-6% |
| Ex 3197 | 2%-4% | 2%-4% | 6%-8% | 6%-8% | 6%-8% |
| Ex 3198 | 2%-4% | 2%-4% | 6%-8% | 6%-8% | 8%-10% |
| Ex 3199 | 2%-4% | 2%-4% | 6%-8% | 8%-10% | 0%-1% |
| Ex 3200 | 2%-4% | 2%-4% | 6%-8% | 8%-10% | 1%-2% |
| Ex 3201 | 2%-4% | 2%-4% | 6%-8% | 8%-10% | 2%-4% |
| Ex 3202 | 2%-4% | 2%-4% | 6%-8% | 8%-10% | 4%-6% |
| Ex 3203 | 2%-4% | 2%-4% | 6%-8% | 8%-10% | 6%-8% |
| Ex 3204 | 2%-4% | 2%-4% | 6%-8% | 8%-10% | 8%-10% |
| Ex 3205 | 2%-4% | 2%-4% | 8%-10% | 0%-1% | 0%-1% |
| Ex 3206 | 2%-4% | 2%-4% | 8%-10% | 0%-1% | 1%-2% |
| Ex 3207 | 2%-4% | 2%-4% | 8%-10% | 0%-1% | 2%-4% |
| Ex 3208 | 2%-4% | 2%-4% | 8%-10% | 0%-1% | 4%-6% |
| Ex 3209 | 2%-4% | 2%-4% | 8%-10% | 0%-1% | 6%-8% |
| Ex 3210 | 2%-4% | 2%-4% | 8%-10% | 0%-1% | 8%-10% |
| Ex 3211 | 2%-4% | 2%-4% | 8%-10% | 1%-2% | 0%-1% |
| Ex 3212 | 2%-4% | 2%-4% | 8%-10% | 1%-2% | 1%-2% |
| Ex 3213 | 2%-4% | 2%-4% | 8%-10% | 1%-2% | 2%-4% |
| Ex 3214 | 2%-4% | 2%-4% | 8%-10% | 1%-2% | 4%-6% |
| Ex 3215 | 2%-4% | 2%-4% | 8%-10% | 1%-2% | 6%-8% |
| Ex 3216 | 2%-4% | 2%-4% | 8%-10% | 1%-2% | 8%-10% |
| Ex 3217 | 2%-4% | 2%-4% | 8%-10% | 2%-4% | 0%-1% |
| Ex 3218 | 2%-4% | 2%-4% | 8%-10% | 2%-4% | 1%-2% |
| Ex 3219 | 2%-4% | 2%-4% | 8%-10% | 2%-4% | 2%-4% |
| Ex 3220 | 2%-4% | 2%-4% | 8%-10% | 2%-4% | 4%-6% |
| Ex 3221 | 2%-4% | 2%-4% | 8%-10% | 2%-4% | 6%-8% |
| Ex 3222 | 2%-4% | 2%-4% | 8%-10% | 2%-4% | 8%-10% |
| Ex 3223 | 2%-4% | 2%-4% | 8%-10% | 4%-6% | 0%-1% |
| Ex 3224 | 2%-4% | 2%-4% | 8%-10% | 4%-6% | 1%-2% |
| Ex 3225 | 2%-4% | 2%-4% | 8%-10% | 4%-6% | 2%-4% |
| Ex 3226 | 2%-4% | 2%-4% | 8%-10% | 4%-6% | 4%-6% |
| Ex 3227 | 2%-4% | 2%-4% | 8%-10% | 4%-6% | 6%-8% |
| Ex 3228 | 2%-4% | 2%-4% | 8%-10% | 4%-6% | 8%-10% |
| Ex 3229 | 2%-4% | 2%-4% | 8%-10% | 6%-8% | 0%-1% |
| Ex 3230 | 2%-4% | 2%-4% | 8%-10% | 6%-8% | 1%-2% |
| Ex 3231 | 2%-4% | 2%-4% | 8%-10% | 6%-8% | 2%-4% |
| Ex 3232 | 2%-4% | 2%-4% | 8%-10% | 6%-8% | 4%-6% |
| Ex 3233 | 2%-4% | 2%-4% | 8%-10% | 6%-8% | 6%-8% |
| Ex 3234 | 2%-4% | 2%-4% | 8%-10% | 6%-8% | 8%-10% |
| Ex 3235 | 2%-4% | 2%-4% | 8%-10% | 8%-10% | 0%-1% |
| Ex 3236 | 2%-4% | 2%-4% | 8%-10% | 8%-10% | 1%-2% |
| Ex 3237 | 2%-4% | 2%-4% | 8%-10% | 8%-10% | 2%-4% |
| Ex 3238 | 2%-4% | 2%-4% | 8%-10% | 8%-10% | 4%-6% |
| Ex 3239 | 2%-4% | 2%-4% | 8%-10% | 8%-10% | 6%-8% |
| Ex 3240 | 2%-4% | 2%-4% | 8%-10% | 8%-10% | 8%-10% |
| Ex 3241 | 2%-4% | 4%-6% | 0%-1% | 0%-1% | 0%-1% |
| Ex 3242 | 2%-4% | 4%-6% | 0%-1% | 0%-1% | 1%-2% |
| Ex 3243 | 2%-4% | 4%-6% | 0%-1% | 0%-1% | 2%-4% |
| Ex 3244 | 2%-4% | 4%-6% | 0%-1% | 0%-1% | 4%-6% |
| Ex 3245 | 2%-4% | 4%-6% | 0%-1% | 0%-1% | 6%-8% |
| Ex 3246 | 2%-4% | 4%-6% | 0%-1% | 0%-1% | 8%-10% |
| Ex 3247 | 2%-4% | 4%-6% | 0%-1% | 1%-2% | 0%-1% |
| Ex 3248 | 2%-4% | 4%-6% | 0%-1% | 1%-2% | 1%-2% |
| Ex 3249 | 2%-4% | 4%-6% | 0%-1% | 1%-2% | 2%-4% |
| Ex 3250 | 2%-4% | 4%-6% | 0%-1% | 1%-2% | 4%-6% |
| Ex 3251 | 2%-4% | 4%-6% | 0%-1% | 1%-2% | 6%-8% |
| Ex 3252 | 2%-4% | 4%-6% | 0%-1% | 1%-2% | 8%-10% |
| Ex 3253 | 2%-4% | 4%-6% | 0%-1% | 2%-4% | 0%-1% |
| Ex 3254 | 2%-4% | 4%-6% | 0%-1% | 2%-4% | 1%-2% |
| Ex 3255 | 2%-4% | 4%-6% | 0%-1% | 2%-4% | 2%-4% |
| Ex 3256 | 2%-4% | 4%-6% | 0%-1% | 2%-4% | 4%-6% |
| Ex 3257 | 2%-4% | 4%-6% | 0%-1% | 2%-4% | 6%-8% |
| Ex 3258 | 2%-4% | 4%-6% | 0%-1% | 2%-4% | 8%-10% |
| Ex 3259 | 2%-4% | 4%-6% | 0%-1% | 4%-6% | 0%-1% |
| Ex 3260 | 2%-4% | 4%-6% | 0%-1% | 4%-6% | 1%-2% |
| Ex 3261 | 2%-4% | 4%-6% | 0%-1% | 4%-6% | 2%-4% |
| Ex 3262 | 2%-4% | 4%-6% | 0%-1% | 4%-6% | 4%-6% |
| Ex 3263 | 2%-4% | 4%-6% | 0%-1% | 4%-6% | 6%-8% |
| Ex 3264 | 2%-4% | 4%-6% | 0%-1% | 4%-6% | 8%-10% |
| Ex 3265 | 2%-4% | 4%-6% | 0%-1% | 6%-8% | 0%-1% |
| Ex 3266 | 2%-4% | 4%-6% | 0%-1% | 6%-8% | 1%-2% |
| Ex 3267 | 2%-4% | 4%-6% | 0%-1% | 6%-8% | 2%-4% |
| Ex 3268 | 2%-4% | 4%-6% | 0%-1% | 6%-8% | 4%-6% |
| Ex 3269 | 2%-4% | 4%-6% | 0%-1% | 6%-8% | 6%-8% |
| Ex 3270 | 2%-4% | 4%-6% | 0%-1% | 6%-8% | 8%-10% |
| Ex 3271 | 2%-4% | 4%-6% | 0%-1% | 8%-10% | 0%-1% |
| Ex 3272 | 2%-4% | 4%-6% | 0%-1% | 8%-10% | 1%-2% |
| Ex 3273 | 2%-4% | 4%-6% | 0%-1% | 8%-10% | 2%-4% |
| Ex 3274 | 2%-4% | 4%-6% | 0%-1% | 8%-10% | 4%-6% |
| Ex 3275 | 2%-4% | 4%-6% | 0%-1% | 8%-10% | 6%-8% |
| Ex 3276 | 2%-4% | 4%-6% | 0%-1% | 8%-10% | 8%-10% |
| Ex 3277 | 2%-4% | 4%-6% | 1%-2% | 0%-1% | 0%-1% |
| Ex 3278 | 2%-4% | 4%-6% | 1%-2% | 0%-1% | 1%-2% |
| Ex 3279 | 2%-4% | 4%-6% | 1%-2% | 0%-1% | 2%-4% |
| Ex 3280 | 2%-4% | 4%-6% | 1%-2% | 0%-1% | 4%-6% |
| Ex 3281 | 2%-4% | 4%-6% | 1%-2% | 0%-1% | 6%-8% |
| Ex 3282 | 2%-4% | 4%-6% | 1%-2% | 0%-1% | 8%-10% |
| Ex 3283 | 2%-4% | 4%-6% | 1%-2% | 1%-2% | 0%-1% |
| Ex 3284 | 2%-4% | 4%-6% | 1%-2% | 1%-2% | 1%-2% |
| Ex 3285 | 2%-4% | 4%-6% | 1%-2% | 1%-2% | 2%-4% |
| Ex 3286 | 2%-4% | 4%-6% | 1%-2% | 1%-2% | 4%-6% |
| Ex 3287 | 2%-4% | 4%-6% | 1%-2% | 1%-2% | 6%-8% |
| Ex 3288 | 2%-4% | 4%-6% | 1%-2% | 1%-2% | 8%-10% |
| Ex 3289 | 2%-4% | 4%-6% | 1%-2% | 2%-4% | 0%-1% |
| Ex 3290 | 2%-4% | 4%-6% | 1%-2% | 2%-4% | 1%-2% |
| Ex 3291 | 2%-4% | 4%-6% | 1%-2% | 2%-4% | 2%-4% |
| Ex 3292 | 2%-4% | 4%-6% | 1%-2% | 2%-4% | 4%-6% |
| Ex 3293 | 2%-4% | 4%-6% | 1%-2% | 2%-4% | 6%-8% |
| Ex 3294 | 2%-4% | 4%-6% | 1%-2% | 2%-4% | 8%-10% |
| Ex 3295 | 2%-4% | 4%-6% | 1%-2% | 4%-6% | 0%-1% |
| Ex 3296 | 2%-4% | 4%-6% | 1%-2% | 4%-6% | 1%-2% |
| Ex 3297 | 2%-4% | 4%-6% | 1%-2% | 4%-6% | 2%-4% |
| Ex 3298 | 2%-4% | 4%-6% | 1%-2% | 4%-6% | 4%-6% |
| Ex 3299 | 2%-4% | 4%-6% | 1%-2% | 4%-6% | 6%-8% |
| Ex 3300 | 2%-4% | 4%-6% | 1%-2% | 4%-6% | 8%-10% |
| Ex 3301 | 2%-4% | 4%-6% | 1%-2% | 6%-8% | 0%-1% |
| Ex 3302 | 2%-4% | 4%-6% | 1%-2% | 6%-8% | 1%-2% |
| Ex 3303 | 2%-4% | 4%-6% | 1%-2% | 6%-8% | 2%-4% |
| Ex 3304 | 2%-4% | 4%-6% | 1%-2% | 6%-8% | 4%-6% |
| Ex 3305 | 2%-4% | 4%-6% | 1%-2% | 6%-8% | 6%-8% |
| Ex 3306 | 2%-4% | 4%-6% | 1%-2% | 6%-8% | 8%-10% |
| Ex 3307 | 2%-4% | 4%-6% | 1%-2% | 8%-10% | 0%-1% |
| Ex 3308 | 2%-4% | 4%-6% | 1%-2% | 8%-10% | 1%-2% |
| Ex 3309 | 2%-4% | 4%-6% | 1%-2% | 8%-10% | 2%-4% |
| Ex 3310 | 2%-4% | 4%-6% | 1%-2% | 8%-10% | 4%-6% |
| Ex 3311 | 2%-4% | 4%-6% | 1%-2% | 8%-10% | 6%-8% |
| Ex 3312 | 2%-4% | 4%-6% | 1%-2% | 8%-10% | 8%-10% |
| Ex 3313 | 2%-4% | 4%-6% | 2%-4% | 0%-1% | 0%-1% |
| Ex 3314 | 2%-4% | 4%-6% | 2%-4% | 0%-1% | 1%-2% |
| Ex 3315 | 2%-4% | 4%-6% | 2%-4% | 0%-1% | 2%-4% |
| Ex 3316 | 2%-4% | 4%-6% | 2%-4% | 0%-1% | 4%-6% |
| Ex 3317 | 2%-4% | 4%-6% | 2%-4% | 0%-1% | 6%-8% |
| Ex 3318 | 2%-4% | 4%-6% | 2%-4% | 0%-1% | 8%-10% |
| Ex 3319 | 2%-4% | 4%-6% | 2%-4% | 1%-2% | 0%-1% |
| Ex 3320 | 2%-4% | 4%-6% | 2%-4% | 1%-2% | 1%-2% |
| Ex 3321 | 2%-4% | 4%-6% | 2%-4% | 1%-2% | 2%-4% |
| Ex 3322 | 2%-4% | 4%-6% | 2%-4% | 1%-2% | 4%-6% |
| Ex 3323 | 2%-4% | 4%-6% | 2%-4% | 1%-2% | 6%-8% |
| Ex 3324 | 2%-4% | 4%-6% | 2%-4% | 1%-2% | 8%-10% |
| Ex 3325 | 2%-4% | 4%-6% | 2%-4% | 2%-4% | 0%-1% |
| Ex 3326 | 2%-4% | 4%-6% | 2%-4% | 2%-4% | 1%-2% |
| Ex 3327 | 2%-4% | 4%-6% | 2%-4% | 2%-4% | 2%-4% |
| Ex 3328 | 2%-4% | 4%-6% | 2%-4% | 2%-4% | 4%-6% |
| Ex 3329 | 2%-4% | 4%-6% | 2%-4% | 2%-4% | 6%-8% |
| Ex 3330 | 2%-4% | 4%-6% | 2%-4% | 2%-4% | 8%-10% |
| Ex 3331 | 2%-4% | 4%-6% | 2%-4% | 4%-6% | 0%-1% |
| Ex 3332 | 2%-4% | 4%-6% | 2%-4% | 4%-6% | 1%-2% |
| Ex 3333 | 2%-4% | 4%-6% | 2%-4% | 4%-6% | 2%-4% |
| Ex 3334 | 2%-4% | 4%-6% | 2%-4% | 4%-6% | 4%-6% |
| Ex 3335 | 2%-4% | 4%-6% | 2%-4% | 4%-6% | 6%-8% |
| Ex 3336 | 2%-4% | 4%-6% | 2%-4% | 4%-6% | 8%-10% |
| Ex 3337 | 2%-4% | 4%-6% | 2%-4% | 6%-8% | 0%-1% |
| Ex 3338 | 2%-4% | 4%-6% | 2%-4% | 6%-8% | 1%-2% |
| Ex 3339 | 2%-4% | 4%-6% | 2%-4% | 6%-8% | 2%-4% |
| Ex 3340 | 2%-4% | 4%-6% | 2%-4% | 6%-8% | 4%-6% |
| Ex 3341 | 2%-4% | 4%-6% | 2%-4% | 6%-8% | 6%-8% |
| Ex 3342 | 2%-4% | 4%-6% | 2%-4% | 6%-8% | 8%-10% |
| Ex 3343 | 2%-4% | 4%-6% | 2%-4% | 8%-10% | 0%-1% |
| Ex 3344 | 2%-4% | 4%-6% | 2%-4% | 8%-10% | 1%-2% |
| Ex 3345 | 2%-4% | 4%-6% | 2%-4% | 8%-10% | 2%-4% |
| Ex 3346 | 2%-4% | 4%-6% | 2%-4% | 8%-10% | 4%-6% |
| Ex 3347 | 2%-4% | 4%-6% | 2%-4% | 8%-10% | 6%-8% |
| Ex 3348 | 2%-4% | 4%-6% | 2%-4% | 8%-10% | 8%-10% |
| Ex 3349 | 2%-4% | 4%-6% | 4%-6% | 0%-1% | 0%-1% |
| Ex 3350 | 2%-4% | 4%-6% | 4%-6% | 0%-1% | 1%-2% |
| Ex 3351 | 2%-4% | 4%-6% | 4%-6% | 0%-1% | 2%-4% |
| Ex 3352 | 2%-4% | 4%-6% | 4%-6% | 0%-1% | 4%-6% |
| Ex 3353 | 2%-4% | 4%-6% | 4%-6% | 0%-1% | 6%-8% |
| Ex 3354 | 2%-4% | 4%-6% | 4%-6% | 0%-1% | 8%-10% |
| Ex 3355 | 2%-4% | 4%-6% | 4%-6% | 1%-2% | 0%-1% |
| Ex 3356 | 2%-4% | 4%-6% | 4%-6% | 1%-2% | 1%-2% |
| Ex 3357 | 2%-4% | 4%-6% | 4%-6% | 1%-2% | 2%-4% |
| Ex 3358 | 2%-4% | 4%-6% | 4%-6% | 1%-2% | 4%-6% |
| Ex 3359 | 2%-4% | 4%-6% | 4%-6% | 1%-2% | 6%-8% |
| Ex 3360 | 2%-4% | 4%-6% | 4%-6% | 1%-2% | 8%-10% |
| Ex 3361 | 2%-4% | 4%-6% | 4%-6% | 2%-4% | 0%-1% |
| Ex 3362 | 2%-4% | 4%-6% | 4%-6% | 2%-4% | 1%-2% |
| Ex 3363 | 2%-4% | 4%-6% | 4%-6% | 2%-4% | 2%-4% |
| Ex 3364 | 2%-4% | 4%-6% | 4%-6% | 2%-4% | 4%-6% |
| Ex 3365 | 2%-4% | 4%-6% | 4%-6% | 2%-4% | 6%-8% |
| Ex 3366 | 2%-4% | 4%-6% | 4%-6% | 2%-4% | 8%-10% |
| Ex 3367 | 2%-4% | 4%-6% | 4%-6% | 4%-6% | 0%-1% |
| Ex 3368 | 2%-4% | 4%-6% | 4%-6% | 4%-6% | 1%-2% |
| Ex 3369 | 2%-4% | 4%-6% | 4%-6% | 4%-6% | 2%-4% |
| Ex 3370 | 2%-4% | 4%-6% | 4%-6% | 4%-6% | 4%-6% |
| Ex 3371 | 2%-4% | 4%-6% | 4%-6% | 4%-6% | 6%-8% |
| Ex 3372 | 2%-4% | 4%-6% | 4%-6% | 4%-6% | 8%-10% |
| Ex 3373 | 2%-4% | 4%-6% | 4%-6% | 6%-8% | 0%-1% |
| Ex 3374 | 2%-4% | 4%-6% | 4%-6% | 6%-8% | 1%-2% |
| Ex 3375 | 2%-4% | 4%-6% | 4%-6% | 6%-8% | 2%-4% |
| Ex 3376 | 2%-4% | 4%-6% | 4%-6% | 6%-8% | 4%-6% |
| Ex 3377 | 2%-4% | 4%-6% | 4%-6% | 6%-8% | 6%-8% |
| Ex 3378 | 2%-4% | 4%-6% | 4%-6% | 6%-8% | 8%-10% |
| Ex 3379 | 2%-4% | 4%-6% | 4%-6% | 8%-10% | 0%-1% |
| Ex 3380 | 2%-4% | 4%-6% | 4%-6% | 8%-10% | 1%-2% |
| Ex 3381 | 2%-4% | 4%-6% | 4%-6% | 8%-10% | 2%-4% |
| Ex 3382 | 2%-4% | 4%-6% | 4%-6% | 8%-10% | 4%-6% |
| Ex 3383 | 2%-4% | 4%-6% | 4%-6% | 8%-10% | 6%-8% |
| Ex 3384 | 2%-4% | 4%-6% | 4%-6% | 8%-10% | 8%-10% |
| Ex 3385 | 2%-4% | 4%-6% | 6%-8% | 0%-1% | 0%-1% |
| Ex 3386 | 2%-4% | 4%-6% | 6%-8% | 0%-1% | 1%-2% |
| Ex 3387 | 2%-4% | 4%-6% | 6%-8% | 0%-1% | 2%-4% |
| Ex 3388 | 2%-4% | 4%-6% | 6%-8% | 0%-1% | 4%-6% |
| Ex 3389 | 2%-4% | 4%-6% | 6%-8% | 0%-1% | 6%-8% |
| Ex 3390 | 2%-4% | 4%-6% | 6%-8% | 0%-1% | 8%-10% |
| Ex 3391 | 2%-4% | 4%-6% | 6%-8% | 1%-2% | 0%-1% |
| Ex 3392 | 2%-4% | 4%-6% | 6%-8% | 1%-2% | 1%-2% |
| Ex 3393 | 2%-4% | 4%-6% | 6%-8% | 1%-2% | 2%-4% |
| Ex 3394 | 2%-4% | 4%-6% | 6%-8% | 1%-2% | 4%-6% |
| Ex 3395 | 2%-4% | 4%-6% | 6%-8% | 1%-2% | 6%-8% |
| Ex 3396 | 2%-4% | 4%-6% | 6%-8% | 1%-2% | 8%-10% |
| Ex 3397 | 2%-4% | 4%-6% | 6%-8% | 2%-4% | 0%-1% |
| Ex 3398 | 2%-4% | 4%-6% | 6%-8% | 2%-4% | 1%-2% |
| Ex 3399 | 2%-4% | 4%-6% | 6%-8% | 2%-4% | 2%-4% |
| Ex 3400 | 2%-4% | 4%-6% | 6%-8% | 2%-4% | 4%-6% |
| Ex 3401 | 2%-4% | 4%-6% | 6%-8% | 2%-4% | 6%-8% |
| Ex 3402 | 2%-4% | 4%-6% | 6%-8% | 2%-4% | 8%-10% |
| Ex 3403 | 2%-4% | 4%-6% | 6%-8% | 4%-6% | 0%-1% |
| Ex 3404 | 2%-4% | 4%-6% | 6%-8% | 4%-6% | 1%-2% |
| Ex 3405 | 2%-4% | 4%-6% | 6%-8% | 4%-6% | 2%-4% |
| Ex 3406 | 2%-4% | 4%-6% | 6%-8% | 4%-6% | 4%-6% |
| Ex 3407 | 2%-4% | 4%-6% | 6%-8% | 4%-6% | 6%-8% |
| Ex 3408 | 2%-4% | 4%-6% | 6%-8% | 4%-6% | 8%-10% |
| Ex 3409 | 2%-4% | 4%-6% | 6%-8% | 6%-8% | 0%-1% |
| Ex 3410 | 2%-4% | 4%-6% | 6%-8% | 6%-8% | 1%-2% |
| Ex 3411 | 2%-4% | 4%-6% | 6%-8% | 6%-8% | 2%-4% |
| Ex 3412 | 2%-4% | 4%-6% | 6%-8% | 6%-8% | 4%-6% |
| Ex 3413 | 2%-4% | 4%-6% | 6%-8% | 6%-8% | 6%-8% |
| Ex 3414 | 2%-4% | 4%-6% | 6%-8% | 6%-8% | 8%-10% |
| Ex 3415 | 2%-4% | 4%-6% | 6%-8% | 8%-10% | 0%-1% |
| Ex 3416 | 2%-4% | 4%-6% | 6%-8% | 8%-10% | 1%-2% |
| Ex 3417 | 2%-4% | 4%-6% | 6%-8% | 8%-10% | 2%-4% |
| Ex 3418 | 2%-4% | 4%-6% | 6%-8% | 8%-10% | 4%-6% |
| Ex 3419 | 2%-4% | 4%-6% | 6%-8% | 8%-10% | 6%-8% |
| Ex 3420 | 2%-4% | 4%-6% | 6%-8% | 8%-10% | 8%-10% |
| Ex 3421 | 2%-4% | 4%-6% | 8%-10% | 0%-1% | 0%-1% |
| Ex 3422 | 2%-4% | 4%-6% | 8%-10% | 0%-1% | 1%-2% |
| Ex 3423 | 2%-4% | 4%-6% | 8%-10% | 0%-1% | 2%-4% |
| Ex 3424 | 2%-4% | 4%-6% | 8%-10% | 0%-1% | 4%-6% |
| Ex 3425 | 2%-4% | 4%-6% | 8%-10% | 0%-1% | 6%-8% |
| Ex 3426 | 2%-4% | 4%-6% | 8%-10% | 0%-1% | 8%-10% |
| Ex 3427 | 2%-4% | 4%-6% | 8%-10% | 1%-2% | 0%-1% |
| Ex 3428 | 2%-4% | 4%-6% | 8%-10% | 1%-2% | 1%-2% |
| Ex 3429 | 2%-4% | 4%-6% | 8%-10% | 1%-2% | 2%-4% |
| Ex 3430 | 2%-4% | 4%-6% | 8%-10% | 1%-2% | 4%-6% |
| Ex 3431 | 2%-4% | 4%-6% | 8%-10% | 1%-2% | 6%-8% |
| Ex 3432 | 2%-4% | 4%-6% | 8%-10% | 1%-2% | 8%-10% |
| Ex 3433 | 2%-4% | 4%-6% | 8%-10% | 2%-4% | 0%-1% |
| Ex 3434 | 2%-4% | 4%-6% | 8%-10% | 2%-4% | 1%-2% |
| Ex 3435 | 2%-4% | 4%-6% | 8%-10% | 2%-4% | 2%-4% |
| Ex 3436 | 2%-4% | 4%-6% | 8%-10% | 2%-4% | 4%-6% |
| Ex 3437 | 2%-4% | 4%-6% | 8%-10% | 2%-4% | 6%-8% |
| Ex 3438 | 2%-4% | 4%-6% | 8%-10% | 2%-4% | 8%-10% |
| Ex 3439 | 2%-4% | 4%-6% | 8%-10% | 4%-6% | 0%-1% |
| Ex 3440 | 2%-4% | 4%-6% | 8%-10% | 4%-6% | 1%-2% |
| Ex 3441 | 2%-4% | 4%-6% | 8%-10% | 4%-6% | 2%-4% |
| Ex 3442 | 2%-4% | 4%-6% | 8%-10% | 4%-6% | 4%-6% |
| Ex 3443 | 2%-4% | 4%-6% | 8%-10% | 4%-6% | 6%-8% |
| Ex 3444 | 2%-4% | 4%-6% | 8%-10% | 4%-6% | 8%-10% |
| Ex 3445 | 2%-4% | 4%-6% | 8%-10% | 6%-8% | 0%-1% |
| Ex 3446 | 2%-4% | 4%-6% | 8%-10% | 6%-8% | 1%-2% |
| Ex 3447 | 2%-4% | 4%-6% | 8%-10% | 6%-8% | 2%-4% |
| Ex 3448 | 2%-4% | 4%-6% | 8%-10% | 6%-8% | 4%-6% |
| Ex 3449 | 2%-4% | 4%-6% | 8%-10% | 6%-8% | 6%-8% |
| Ex 3450 | 2%-4% | 4%-6% | 8%-10% | 6%-8% | 8%-10% |
| Ex 3451 | 2%-4% | 4%-6% | 8%-10% | 8%-10% | 0%-1% |
| Ex 3452 | 2%-4% | 4%-6% | 8%-10% | 8%-10% | 1%-2% |
| Ex 3453 | 2%-4% | 4%-6% | 8%-10% | 8%-10% | 2%-4% |
| Ex 3454 | 2%-4% | 4%-6% | 8%-10% | 8%-10% | 4%-6% |
| Ex 3455 | 2%-4% | 4%-6% | 8%-10% | 8%-10% | 6%-8% |
| Ex 3456 | 2%-4% | 4%-6% | 8%-10% | 8%-10% | 8%-10% |
| Ex 3457 | 2%-4% | 6%-8% | 0%-1% | 0%-1% | 0%-1% |
| Ex 3458 | 2%-4% | 6%-8% | 0%-1% | 0%-1% | 1%-2% |
| Ex 3459 | 2%-4% | 6%-8% | 0%-1% | 0%-1% | 2%-4% |
| Ex 3460 | 2%-4% | 6%-8% | 0%-1% | 0%-1% | 4%-6% |
| Ex 3461 | 2%-4% | 6%-8% | 0%-1% | 0%-1% | 6%-8% |
| Ex 3462 | 2%-4% | 6%-8% | 0%-1% | 0%-1% | 8%-10% |
| Ex 3463 | 2%-4% | 6%-8% | 0%-1% | 1%-2% | 0%-1% |
| Ex 3464 | 2%-4% | 6%-8% | 0%-1% | 1%-2% | 1%-2% |
| Ex 3465 | 2%-4% | 6%-8% | 0%-1% | 1%-2% | 2%-4% |
| Ex 3466 | 2%-4% | 6%-8% | 0%-1% | 1%-2% | 4%-6% |
| Ex 3467 | 2%-4% | 6%-8% | 0%-1% | 1%-2% | 6%-8% |
| Ex 3468 | 2%-4% | 6%-8% | 0%-1% | 1%-2% | 8%-10% |
| Ex 3469 | 2%-4% | 6%-8% | 0%-1% | 2%-4% | 0%-1% |
| Ex 3470 | 2%-4% | 6%-8% | 0%-1% | 2%-4% | 1%-2% |
| Ex 3471 | 2%-4% | 6%-8% | 0%-1% | 2%-4% | 2%-4% |
| Ex 3472 | 2%-4% | 6%-8% | 0%-1% | 2%-4% | 4%-6% |
| Ex 3473 | 2%-4% | 6%-8% | 0%-1% | 2%-4% | 6%-8% |
| Ex 3474 | 2%-4% | 6%-8% | 0%-1% | 2%-4% | 8%-10% |
| Ex 3475 | 2%-4% | 6%-8% | 0%-1% | 4%-6% | 0%-1% |
| Ex 3476 | 2%-4% | 6%-8% | 0%-1% | 4%-6% | 1%-2% |
| Ex 3477 | 2%-4% | 6%-8% | 0%-1% | 4%-6% | 2%-4% |
| Ex 3478 | 2%-4% | 6%-8% | 0%-1% | 4%-6% | 4%-6% |
| Ex 3479 | 2%-4% | 6%-8% | 0%-1% | 4%-6% | 6%-8% |
| Ex 3480 | 2%-4% | 6%-8% | 0%-1% | 4%-6% | 8%-10% |
| Ex 3481 | 2%-4% | 6%-8% | 0%-1% | 6%-8% | 0%-1% |
| Ex 3482 | 2%-4% | 6%-8% | 0%-1% | 6%-8% | 1%-2% |
| Ex 3483 | 2%-4% | 6%-8% | 0%-1% | 6%-8% | 2%-4% |
| Ex 3484 | 2%-4% | 6%-8% | 0%-1% | 6%-8% | 4%-6% |
| Ex 3485 | 2%-4% | 6%-8% | 0%-1% | 6%-8% | 6%-8% |
| Ex 3486 | 2%-4% | 6%-8% | 0%-1% | 6%-8% | 8%-10% |
| Ex 3487 | 2%-4% | 6%-8% | 0%-1% | 8%-10% | 0%-1% |
| Ex 3488 | 2%-4% | 6%-8% | 0%-1% | 8%-10% | 1%-2% |
| Ex 3489 | 2%-4% | 6%-8% | 0%-1% | 8%-10% | 2%-4% |
| Ex 3490 | 2%-4% | 6%-8% | 0%-1% | 8%-10% | 4%-6% |
| Ex 3491 | 2%-4% | 6%-8% | 0%-1% | 8%-10% | 6%-8% |
| Ex 3492 | 2%-4% | 6%-8% | 0%-1% | 8%-10% | 8%-10% |
| Ex 3493 | 2%-4% | 6%-8% | 1%-2% | 0%-1% | 0%-1% |
| Ex 3494 | 2%-4% | 6%-8% | 1%-2% | 0%-1% | 1%-2% |
| Ex 3495 | 2%-4% | 6%-8% | 1%-2% | 0%-1% | 2%-4% |
| Ex 3496 | 2%-4% | 6%-8% | 1%-2% | 0%-1% | 4%-6% |
| Ex 3497 | 2%-4% | 6%-8% | 1%-2% | 0%-1% | 6%-8% |
| Ex 3498 | 2%-4% | 6%-8% | 1%-2% | 0%-1% | 8%-10% |
| Ex 3499 | 2%-4% | 6%-8% | 1%-2% | 1%-2% | 0%-1% |
| Ex 3500 | 2%-4% | 6%-8% | 1%-2% | 1%-2% | 1%-2% |
| Ex 3501 | 2%-4% | 6%-8% | 1%-2% | 1%-2% | 2%-4% |
| Ex 3502 | 2%-4% | 6%-8% | 1%-2% | 1%-2% | 4%-6% |
| Ex 3503 | 2%-4% | 6%-8% | 1%-2% | 1%-2% | 6%-8% |
| Ex 3504 | 2%-4% | 6%-8% | 1%-2% | 1%-2% | 8%-10% |
| Ex 3505 | 2%-4% | 6%-8% | 1%-2% | 2%-4% | 0%-1% |
| Ex 3506 | 2%-4% | 6%-8% | 1%-2% | 2%-4% | 1%-2% |
| Ex 3507 | 2%-4% | 6%-8% | 1%-2% | 2%-4% | 2%-4% |
| Ex 3508 | 2%-4% | 6%-8% | 1%-2% | 2%-4% | 4%-6% |
| Ex 3509 | 2%-4% | 6%-8% | 1%-2% | 2%-4% | 6%-8% |
| Ex 3510 | 2%-4% | 6%-8% | 1%-2% | 2%-4% | 8%-10% |
| Ex 3511 | 2%-4% | 6%-8% | 1%-2% | 4%-6% | 0%-1% |
| Ex 3512 | 2%-4% | 6%-8% | 1%-2% | 4%-6% | 1%-2% |
| Ex 3513 | 2%-4% | 6%-8% | 1%-2% | 4%-6% | 2%-4% |
| Ex 3514 | 2%-4% | 6%-8% | 1%-2% | 4%-6% | 4%-6% |
| Ex 3515 | 2%-4% | 6%-8% | 1%-2% | 4%-6% | 6%-8% |
| Ex 3516 | 2%-4% | 6%-8% | 1%-2% | 4%-6% | 8%-10% |
| Ex 3517 | 2%-4% | 6%-8% | 1%-2% | 6%-8% | 0%-1% |
| Ex 3518 | 2%-4% | 6%-8% | 1%-2% | 6%-8% | 1%-2% |
| Ex 3519 | 2%-4% | 6%-8% | 1%-2% | 6%-8% | 2%-4% |
| Ex 3520 | 2%-4% | 6%-8% | 1%-2% | 6%-8% | 4%-6% |
| Ex 3521 | 2%-4% | 6%-8% | 1%-2% | 6%-8% | 6%-8% |
| Ex 3522 | 2%-4% | 6%-8% | 1%-2% | 6%-8% | 8%-10% |
| Ex 3523 | 2%-4% | 6%-8% | 1%-2% | 8%-10% | 0%-1% |
| Ex 3524 | 2%-4% | 6%-8% | 1%-2% | 8%-10% | 1%-2% |
| Ex 3525 | 2%-4% | 6%-8% | 1%-2% | 8%-10% | 2%-4% |
| Ex 3526 | 2%-4% | 6%-8% | 1%-2% | 8%-10% | 4%-6% |
| Ex 3527 | 2%-4% | 6%-8% | 1%-2% | 8%-10% | 6%-8% |
| Ex 3528 | 2%-4% | 6%-8% | 1%-2% | 8%-10% | 8%-10% |
| Ex 3529 | 2%-4% | 6%-8% | 2%-4% | 0%-1% | 0%-1% |
| Ex 3530 | 2%-4% | 6%-8% | 2%-4% | 0%-1% | 1%-2% |
| Ex 3531 | 2%-4% | 6%-8% | 2%-4% | 0%-1% | 2%-4% |
| Ex 3532 | 2%-4% | 6%-8% | 2%-4% | 0%-1% | 4%-6% |
| Ex 3533 | 2%-4% | 6%-8% | 2%-4% | 0%-1% | 6%-8% |
| Ex 3534 | 2%-4% | 6%-8% | 2%-4% | 0%-1% | 8%-10% |
| Ex 3535 | 2%-4% | 6%-8% | 2%-4% | 1%-2% | 0%-1% |
| Ex 3536 | 2%-4% | 6%-8% | 2%-4% | 1%-2% | 1%-2% |
| Ex 3537 | 2%-4% | 6%-8% | 2%-4% | 1%-2% | 2%-4% |
| Ex 3538 | 2%-4% | 6%-8% | 2%-4% | 1%-2% | 4%-6% |
| Ex 3539 | 2%-4% | 6%-8% | 2%-4% | 1%-2% | 6%-8% |
| Ex 3540 | 2%-4% | 6%-8% | 2%-4% | 1%-2% | 8%-10% |
| Ex 3541 | 2%-4% | 6%-8% | 2%-4% | 2%-4% | 0%-1% |
| Ex 3542 | 2%-4% | 6%-8% | 2%-4% | 2%-4% | 1%-2% |
| Ex 3543 | 2%-4% | 6%-8% | 2%-4% | 2%-4% | 2%-4% |
| Ex 3544 | 2%-4% | 6%-8% | 2%-4% | 2%-4% | 4%-6% |
| Ex 3545 | 2%-4% | 6%-8% | 2%-4% | 2%-4% | 6%-8% |
| Ex 3546 | 2%-4% | 6%-8% | 2%-4% | 2%-4% | 8%-10% |
| Ex 3547 | 2%-4% | 6%-8% | 2%-4% | 4%-6% | 0%-1% |
| Ex 3548 | 2%-4% | 6%-8% | 2%-4% | 4%-6% | 1%-2% |
| Ex 3549 | 2%-4% | 6%-8% | 2%-4% | 4%-6% | 2%-4% |
| Ex 3550 | 2%-4% | 6%-8% | 2%-4% | 4%-6% | 4%-6% |
| Ex 3551 | 2%-4% | 6%-8% | 2%-4% | 4%-6% | 6%-8% |
| Ex 3552 | 2%-4% | 6%-8% | 2%-4% | 4%-6% | 8%-10% |
| Ex 3553 | 2%-4% | 6%-8% | 2%-4% | 6%-8% | 0%-1% |
| Ex 3554 | 2%-4% | 6%-8% | 2%-4% | 6%-8% | 1%-2% |
| Ex 3555 | 2%-4% | 6%-8% | 2%-4% | 6%-8% | 2%-4% |
| Ex 3556 | 2%-4% | 6%-8% | 2%-4% | 6%-8% | 4%-6% |
| Ex 3557 | 2%-4% | 6%-8% | 2%-4% | 6%-8% | 6%-8% |
| Ex 3558 | 2%-4% | 6%-8% | 2%-4% | 6%-8% | 8%-10% |
| Ex 3559 | 2%-4% | 6%-8% | 2%-4% | 8%-10% | 0%-1% |
| Ex 3560 | 2%-4% | 6%-8% | 2%-4% | 8%-10% | 1%-2% |
| Ex 3561 | 2%-4% | 6%-8% | 2%-4% | 8%-10% | 2%-4% |
| Ex 3562 | 2%-4% | 6%-8% | 2%-4% | 8%-10% | 4%-6% |
| Ex 3563 | 2%-4% | 6%-8% | 2%-4% | 8%-10% | 6%-8% |
| Ex 3564 | 2%-4% | 6%-8% | 2%-4% | 8%-10% | 8%-10% |
| Ex 3565 | 2%-4% | 6%-8% | 4%-6% | 0%-1% | 0%-1% |
| Ex 3566 | 2%-4% | 6%-8% | 4%-6% | 0%-1% | 1%-2% |
| Ex 3567 | 2%-4% | 6%-8% | 4%-6% | 0%-1% | 2%-4% |
| Ex 3568 | 2%-4% | 6%-8% | 4%-6% | 0%-1% | 4%-6% |
| Ex 3569 | 2%-4% | 6%-8% | 4%-6% | 0%-1% | 6%-8% |
| Ex 3570 | 2%-4% | 6%-8% | 4%-6% | 0%-1% | 8%-10% |
| Ex 3571 | 2%-4% | 6%-8% | 4%-6% | 1%-2% | 0%-1% |
| Ex 3572 | 2%-4% | 6%-8% | 4%-6% | 1%-2% | 1%-2% |
| Ex 3573 | 2%-4% | 6%-8% | 4%-6% | 1%-2% | 2%-4% |
| Ex 3574 | 2%-4% | 6%-8% | 4%-6% | 1%-2% | 4%-6% |
| Ex 3575 | 2%-4% | 6%-8% | 4%-6% | 1%-2% | 6%-8% |
| Ex 3576 | 2%-4% | 6%-8% | 4%-6% | 1%-2% | 8%-10% |
| Ex 3577 | 2%-4% | 6%-8% | 4%-6% | 2%-4% | 0%-1% |
| Ex 3578 | 2%-4% | 6%-8% | 4%-6% | 2%-4% | 1%-2% |
| Ex 3579 | 2%-4% | 6%-8% | 4%-6% | 2%-4% | 2%-4% |
| Ex 3580 | 2%-4% | 6%-8% | 4%-6% | 2%-4% | 4%-6% |
| Ex 3581 | 2%-4% | 6%-8% | 4%-6% | 2%-4% | 6%-8% |
| Ex 3582 | 2%-4% | 6%-8% | 4%-6% | 2%-4% | 8%-10% |
| Ex 3583 | 2%-4% | 6%-8% | 4%-6% | 4%-6% | 0%-1% |
| Ex 3584 | 2%-4% | 6%-8% | 4%-6% | 4%-6% | 1%-2% |
| Ex 3585 | 2%-4% | 6%-8% | 4%-6% | 4%-6% | 2%-4% |
| Ex 3586 | 2%-4% | 6%-8% | 4%-6% | 4%-6% | 4%-6% |
| Ex 3587 | 2%-4% | 6%-8% | 4%-6% | 4%-6% | 6%-8% |
| Ex 3588 | 2%-4% | 6%-8% | 4%-6% | 4%-6% | 8%-10% |
| Ex 3589 | 2%-4% | 6%-8% | 4%-6% | 6%-8% | 0%-1% |
| Ex 3590 | 2%-4% | 6%-8% | 4%-6% | 6%-8% | 1%-2% |
| Ex 3591 | 2%-4% | 6%-8% | 4%-6% | 6%-8% | 2%-4% |
| Ex 3592 | 2%-4% | 6%-8% | 4%-6% | 6%-8% | 4%-6% |
| Ex 3593 | 2%-4% | 6%-8% | 4%-6% | 6%-8% | 6%-8% |
| Ex 3594 | 2%-4% | 6%-8% | 4%-6% | 6%-8% | 8%-10% |
| Ex 3595 | 2%-4% | 6%-8% | 4%-6% | 8%-10% | 0%-1% |
| Ex 3596 | 2%-4% | 6%-8% | 4%-6% | 8%-10% | 1%-2% |
| Ex 3597 | 2%-4% | 6%-8% | 4%-6% | 8%-10% | 2%-4% |
| Ex 3598 | 2%-4% | 6%-8% | 4%-6% | 8%-10% | 4%-6% |
| Ex 3599 | 2%-4% | 6%-8% | 4%-6% | 8%-10% | 6%-8% |
| Ex 3600 | 2%-4% | 6%-8% | 4%-6% | 8%-10% | 8%-10% |
| Ex 3601 | 2%-4% | 6%-8% | 6%-8% | 0%-1% | 0%-1% |
| Ex 3602 | 2%-4% | 6%-8% | 6%-8% | 0%-1% | 1%-2% |
| Ex 3603 | 2%-4% | 6%-8% | 6%-8% | 0%-1% | 2%-4% |
| Ex 3604 | 2%-4% | 6%-8% | 6%-8% | 0%-1% | 4%-6% |
| Ex 3605 | 2%-4% | 6%-8% | 6%-8% | 0%-1% | 6%-8% |
| Ex 3606 | 2%-4% | 6%-8% | 6%-8% | 0%-1% | 8%-10% |
| Ex 3607 | 2%-4% | 6%-8% | 6%-8% | 1%-2% | 0%-1% |
| Ex 3608 | 2%-4% | 6%-8% | 6%-8% | 1%-2% | 1%-2% |
| Ex 3609 | 2%-4% | 6%-8% | 6%-8% | 1%-2% | 2%-4% |
| Ex 3610 | 2%-4% | 6%-8% | 6%-8% | 1%-2% | 4%-6% |
| Ex 3611 | 2%-4% | 6%-8% | 6%-8% | 1%-2% | 6%-8% |
| Ex 3612 | 2%-4% | 6%-8% | 6%-8% | 1%-2% | 8%-10% |
| Ex 3613 | 2%-4% | 6%-8% | 6%-8% | 2%-4% | 0%-1% |
| Ex 3614 | 2%-4% | 6%-8% | 6%-8% | 2%-4% | 1%-2% |
| Ex 3615 | 2%-4% | 6%-8% | 6%-8% | 2%-4% | 2%-4% |
| Ex 3616 | 2%-4% | 6%-8% | 6%-8% | 2%-4% | 4%-6% |
| Ex 3617 | 2%-4% | 6%-8% | 6%-8% | 2%-4% | 6%-8% |
| Ex 3618 | 2%-4% | 6%-8% | 6%-8% | 2%-4% | 8%-10% |
| Ex 3619 | 2%-4% | 6%-8% | 6%-8% | 4%-6% | 0%-1% |
| Ex 3620 | 2%-4% | 6%-8% | 6%-8% | 4%-6% | 1%-2% |
| Ex 3621 | 2%-4% | 6%-8% | 6%-8% | 4%-6% | 2%-4% |
| Ex 3622 | 2%-4% | 6%-8% | 6%-8% | 4%-6% | 4%-6% |
| Ex 3623 | 2%-4% | 6%-8% | 6%-8% | 4%-6% | 6%-8% |
| Ex 3624 | 2%-4% | 6%-8% | 6%-8% | 4%-6% | 8%-10% |
| Ex 3625 | 2%-4% | 6%-8% | 6%-8% | 6%-8% | 0%-1% |
| Ex 3626 | 2%-4% | 6%-8% | 6%-8% | 6%-8% | 1%-2% |
| Ex 3627 | 2%-4% | 6%-8% | 6%-8% | 6%-8% | 2%-4% |
| Ex 3628 | 2%-4% | 6%-8% | 6%-8% | 6%-8% | 4%-6% |
| Ex 3629 | 2%-4% | 6%-8% | 6%-8% | 6%-8% | 6%-8% |
| Ex 3630 | 2%-4% | 6%-8% | 6%-8% | 6%-8% | 8%-10% |
| Ex 3631 | 2%-4% | 6%-8% | 6%-8% | 8%-10% | 0%-1% |
| Ex 3632 | 2%-4% | 6%-8% | 6%-8% | 8%-10% | 1%-2% |
| Ex 3633 | 2%-4% | 6%-8% | 6%-8% | 8%-10% | 2%-4% |
| Ex 3534 | 2%-4% | 6%-8% | 6%-8% | 8%-10% | 4%-6% |
| Ex 3635 | 2%-4% | 6%-8% | 6%-8% | 8%-10% | 6%-8% |
| Ex 3636 | 2%-4% | 6%-8% | 6%-8% | 8%-10% | 8%-10% |
| Ex 3637 | 2%-4% | 6%-8% | 8%-10% | 0%-1% | 0%-1% |
| Ex 3638 | 2%-4% | 6%-8% | 8%-10% | 0%-1% | 1%-2% |
| Ex 3639 | 2%-4% | 6%-8% | 8%-10% | 0%-1% | 2%-4% |
| Ex 3640 | 2%-4% | 6%-8% | 8%-10% | 0%-1% | 4%-6% |
| Ex 3641 | 2%-4% | 6%-8% | 8%-10% | 0%-1% | 6%-8% |
| Ex 3642 | 2%-4% | 6%-8% | 8%-10% | 0%-1% | 8%-10% |
| Ex 3643 | 2%-4% | 6%-8% | 8%-10% | 1%-2% | 0%-1% |
| Ex 3644 | 2%-4% | 6%-8% | 8%-10% | 1%-2% | 1%-2% |
| Ex 3645 | 2%-4% | 6%-8% | 8%-10% | 1%-2% | 2%-4% |
| Ex 3646 | 2%-4% | 6%-8% | 8%-10% | 1%-2% | 4%-6% |
| Ex 3647 | 2%-4% | 6%-8% | 8%-10% | 1%-2% | 6%-8% |
| Ex 3648 | 2%-4% | 6%-8% | 8%-10% | 1%-2% | 8%-10% |
| Ex 3649 | 2%-4% | 6%-8% | 8%-10% | 2%-4% | 0%-1% |
| Ex 3650 | 2%-4% | 6%-8% | 8%-10% | 2%-4% | 1%-2% |
| Ex 3651 | 2%-4% | 6%-8% | 8%-10% | 2%-4% | 2%-4% |
| Ex 3652 | 2%-4% | 6%-8% | 8%-10% | 2%-4% | 4%-6% |
| Ex 3653 | 2%-4% | 6%-8% | 8%-10% | 2%-4% | 6%-8% |
| Ex 3654 | 2%-4% | 6%-8% | 8%-10% | 2%-4% | 8%-10% |
| Ex 3655 | 2%-4% | 6%-8% | 8%-10% | 4%-6% | 0%-1% |
| Ex 3656 | 2%-4% | 6%-8% | 8%-10% | 4%-6% | 1%-2% |
| Ex 3657 | 2%-4% | 6%-8% | 8%-10% | 4%-6% | 2%-4% |
| Ex 3658 | 2%-4% | 6%-8% | 8%-10% | 4%-6% | 4%-6% |
| Ex 3659 | 2%-4% | 6%-8% | 8%-10% | 4%-6% | 6%-8% |
| Ex 3660 | 2%-4% | 6%-8% | 8%-10% | 4%-6% | 8%-10% |
| Ex 3661 | 2%-4% | 6%-8% | 8%-10% | 6%-8% | 0%-1% |
| Ex 3662 | 2%-4% | 6%-8% | 8%-10% | 6%-8% | 1%-2% |
| Ex 3663 | 2%-4% | 6%-8% | 8%-10% | 6%-8% | 2%-4% |
| Ex 3664 | 2%-4% | 6%-8% | 8%-10% | 6%-8% | 4%-6% |
| Ex 3665 | 2%-4% | 6%-8% | 8%-10% | 6%-8% | 6%-8% |
| Ex 3666 | 2%-4% | 6%-8% | 8%-10% | 6%-8% | 8%-10% |
| Ex 3667 | 2%-4% | 6%-8% | 8%-10% | 8%-10% | 0%-1% |
| Ex 3668 | 2%-4% | 6%-8% | 8%-10% | 8%-10% | 1%-2% |
| Ex 3669 | 2%-4% | 6%-8% | 8%-10% | 8%-10% | 2%-4% |
| Ex 3670 | 2%-4% | 6%-8% | 8%-10% | 8%-10% | 4%-6% |
| Ex 3671 | 2%-4% | 6%-8% | 8%-10% | 8%-10% | 6%-8% |
| Ex 3672 | 2%-4% | 6%-8% | 8%-10% | 8%-10% | 8%-10% |
| Ex 3673 | 2%-4% | 8%-10% | 0%-1% | 0%-1% | 0%-1% |
| Ex 3674 | 2%-4% | 8%-10% | 0%-1% | 0%-1% | 1%-2% |
| Ex 3675 | 2%-4% | 8%-10% | 0%-1% | 0%-1% | 2%-4% |
| Ex 3676 | 2%-4% | 8%-10% | 0%-1% | 0%-1% | 4%-6% |
| Ex 3677 | 2%-4% | 8%-10% | 0%-1% | 0%-1% | 6%-8% |
| Ex 3678 | 2%-4% | 8%-10% | 0%-1% | 0%-1% | 8%-10% |
| Ex 3679 | 2%-4% | 8%-10% | 0%-1% | 1%-2% | 0%-1% |
| Ex 3680 | 2%-4% | 8%-10% | 0%-1% | 1%-2% | 1%-2% |
| Ex 3681 | 2%-4% | 8%-10% | 0%-1% | 1%-2% | 2%-4% |
| Ex 3682 | 2%-4% | 8%-10% | 0%-1% | 1%-2% | 4%-6% |
| Ex 3683 | 2%-4% | 8%-10% | 0%-1% | 1%-2% | 6%-8% |
| Ex 3684 | 2%-4% | 8%-10% | 0%-1% | 1%-2% | 8%-10% |
| Ex 3685 | 2%-4% | 8%-10% | 0%-1% | 2%-4% | 0%-1% |
| Ex 3686 | 2%-4% | 8%-10% | 0%-1% | 2%-4% | 1%-2% |
| Ex 3687 | 2%-4% | 8%-10% | 0%-1% | 2%-4% | 2%-4% |
| Ex 3688 | 2%-4% | 8%-10% | 0%-1% | 2%-4% | 4%-6% |
| Ex 3689 | 2%-4% | 8%-10% | 0%-1% | 2%-4% | 6%-8% |
| Ex 3690 | 2%-4% | 8%-10% | 0%-1% | 2%-4% | 8%-10% |
| Ex 3691 | 2%-4% | 8%-10% | 0%-1% | 4%-6% | 0%-1% |
| Ex 3692 | 2%-4% | 8%-10% | 0%-1% | 4%-6% | 1%-2% |
| Ex 3693 | 2%-4% | 8%-10% | 0%-1% | 4%-6% | 2%-4% |
| Ex 3694 | 2%-4% | 8%-10% | 0%-1% | 4%-6% | 4%-6% |
| Ex 3695 | 2%-4% | 8%-10% | 0%-1% | 4%-6% | 6%-8% |
| Ex 3696 | 2%-4% | 8%-10% | 0%-1% | 4%-6% | 8%-10% |
| Ex 3697 | 2%-4% | 8%-10% | 0%-1% | 6%-8% | 0%-1% |
| Ex 3698 | 2%-4% | 8%-10% | 0%-1% | 6%-8% | 1%-2% |
| Ex 3699 | 2%-4% | 8%-10% | 0%-1% | 6%-8% | 2%-4% |
| Ex 3700 | 2%-4% | 8%-10% | 0%-1% | 6%-8% | 4%-6% |
| Ex 3701 | 2%-4% | 8%-10% | 0%-1% | 6%-8% | 6%-8% |
| Ex 3702 | 2%-4% | 8%-10% | 0%-1% | 6%-8% | 8%-10% |
| Ex 3703 | 2%-4% | 8%-10% | 0%-1% | 8%-10% | 0%-1% |
| Ex 3704 | 2%-4% | 8%-10% | 0%-1% | 8%-10% | 1%-2% |
| Ex 3705 | 2%-4% | 8%-10% | 0%-1% | 8%-10% | 2%-4% |
| Ex 3706 | 2%-4% | 8%-10% | 0%-1% | 8%-10% | 4%-6% |
| Ex 3707 | 2%-4% | 8%-10% | 0%-1% | 8%-10% | 6%-8% |
| Ex 3708 | 2%-4% | 8%-10% | 0%-1% | 8%-10% | 8%-10% |
| Ex 3709 | 2%-4% | 8%-10% | 1%-2% | 0%-1% | 0%-1% |
| Ex 3710 | 2%-4% | 8%-10% | 1%-2% | 0%-1% | 1%-2% |
| Ex 3711 | 2%-4% | 8%-10% | 1%-2% | 0%-1% | 2%-4% |
| Ex 3712 | 2%-4% | 8%-10% | 1%-2% | 0%-1% | 4%-6% |
| Ex 3713 | 2%-4% | 8%-10% | 1%-2% | 0%-1% | 6%-8% |
| Ex 3714 | 2%-4% | 8%-10% | 1%-2% | 0%-1% | 8%-10% |
| Ex 3715 | 2%-4% | 8%-10% | 1%-2% | 1%-2% | 0%-1% |
| Ex 3716 | 2%-4% | 8%-10% | 1%-2% | 1%-2% | 1%-2% |
| Ex 3717 | 2%-4% | 8%-10% | 1%-2% | 1%-2% | 2%-4% |
| Ex 3718 | 2%-4% | 8%-10% | 1%-2% | 1%-2% | 4%-6% |
| Ex 3719 | 2%-4% | 8%-10% | 1%-2% | 1%-2% | 6%-8% |
| Ex 3720 | 2%-4% | 8%-10% | 1%-2% | 1%-2% | 8%-10% |
| Ex 3721 | 2%-4% | 8%-10% | 1%-2% | 2%-4% | 0%-1% |
| Ex 3722 | 2%-4% | 8%-10% | 1%-2% | 2%-4% | 1%-2% |
| Ex 3723 | 2%-4% | 8%-10% | 1%-2% | 2%-4% | 2%-4% |
| Ex 3724 | 2%-4% | 8%-10% | 1%-2% | 2%-4% | 4%-6% |
| Ex 3725 | 2%-4% | 8%-10% | 1%-2% | 2%-4% | 6%-8% |
| Ex 3726 | 2%-4% | 8%-10% | 1%-2% | 2%-4% | 8%-10% |
| Ex 3727 | 2%-4% | 8%-10% | 1%-2% | 4%-6% | 0%-1% |
| Ex 3728 | 2%-4% | 8%-10% | 1%-2% | 4%-6% | 1%-2% |
| Ex 3729 | 2%-4% | 8%-10% | 1%-2% | 4%-6% | 2%-4% |
| Ex 3730 | 2%-4% | 8%-10% | 1%-2% | 4%-6% | 4%-6% |
| Ex 3731 | 2%-4% | 8%-10% | 1%-2% | 4%-6% | 6%-8% |
| Ex 3732 | 2%-4% | 8%-10% | 1%-2% | 4%-6% | 8%-10% |
| Ex 3733 | 2%-4% | 8%-10% | 1%-2% | 6%-8% | 0%-1% |
| Ex 3734 | 2%-4% | 8%-10% | 1%-2% | 6%-8% | 1%-2% |
| Ex 3735 | 2%-4% | 8%-10% | 1%-2% | 6%-8% | 2%-4% |
| Ex 3736 | 2%-4% | 8%-10% | 1%-2% | 6%-8% | 4%-6% |
| Ex 3737 | 2%-4% | 8%-10% | 1%-2% | 6%-8% | 6%-8% |
| Ex 3738 | 2%-4% | 8%-10% | 1%-2% | 6%-8% | 8%-10% |
| Ex 3739 | 2%-4% | 8%-10% | 1%-2% | 8%-10% | 0%-1% |
| Ex 3740 | 2%-4% | 8%-10% | 1%-2% | 8%-10% | 1%-2% |
| Ex 3741 | 2%-4% | 8%-10% | 1%-2% | 8%-10% | 2%-4% |
| Ex 3742 | 2%-4% | 8%-10% | 1%-2% | 8%-10% | 4%-6% |
| Ex 3743 | 2%-4% | 8%-10% | 1%-2% | 8%-10% | 6%-8% |
| Ex 3744 | 2%-4% | 8%-10% | 1%-2% | 8%-10% | 8%-10% |
| Ex 3745 | 2%-4% | 8%-10% | 2%-4% | 0%-1% | 0%-1% |
| Ex 3746 | 2%-4% | 8%-10% | 2%-4% | 0%-1% | 1%-2% |
| Ex 3747 | 2%-4% | 8%-10% | 2%-4% | 0%-1% | 2%-4% |
| Ex 3748 | 2%-4% | 8%-10% | 2%-4% | 0%-1% | 4%-6% |
| Ex 3749 | 2%-4% | 8%-10% | 2%-4% | 0%-1% | 6%-8% |
| Ex 3750 | 2%-4% | 8%-10% | 2%-4% | 0%-1% | 8%-10% |
| Ex 3751 | 2%-4% | 8%-10% | 2%-4% | 1%-2% | 0%-1% |
| Ex 3752 | 2%-4% | 8%-10% | 2%-4% | 1%-2% | 1%-2% |
| Ex 3753 | 2%-4% | 8%-10% | 2%-4% | 1%-2% | 2%-4% |
| Ex 3754 | 2%-4% | 8%-10% | 2%-4% | 1%-2% | 4%-6% |
| Ex 3755 | 2%-4% | 8%-10% | 2%-4% | 1%-2% | 6%-8% |
| Ex 3756 | 2%-4% | 8%-10% | 2%-4% | 1%-2% | 8%-10% |
| Ex 3757 | 2%-4% | 8%-10% | 2%-4% | 2%-4% | 0%-1% |
| Ex 3758 | 2%-4% | 8%-10% | 2%-4% | 2%-4% | 1%-2% |
| Ex 3759 | 2%-4% | 8%-10% | 2%-4% | 2%-4% | 2%-4% |
| Ex 3760 | 2%-4% | 8%-10% | 2%-4% | 2%-4% | 4%-6% |
| Ex 3761 | 2%-4% | 8%-10% | 2%-4% | 2%-4% | 6%-8% |
| Ex 3762 | 2%-4% | 8%-10% | 2%-4% | 2%-4% | 8%-10% |
| Ex 3763 | 2%-4% | 8%-10% | 2%-4% | 4%-6% | 0%-1% |
| Ex 3764 | 2%-4% | 8%-10% | 2%-4% | 4%-6% | 1%-2% |
| Ex 3765 | 2%-4% | 8%-10% | 2%-4% | 4%-6% | 2%-4% |
| Ex 3766 | 2%-4% | 8%-10% | 2%-4% | 4%-6% | 4%-6% |
| Ex 3767 | 2%-4% | 8%-10% | 2%-4% | 4%-6% | 6%-8% |
| Ex 3768 | 2%-4% | 8%-10% | 2%-4% | 4%-6% | 8%-10% |
| Ex 3769 | 2%-4% | 8%-10% | 2%-4% | 6%-8% | 0%-1% |
| Ex 3770 | 2%-4% | 8%-10% | 2%-4% | 6%-8% | 1%-2% |
| Ex 3771 | 2%-4% | 8%-10% | 2%-4% | 6%-8% | 2%-4% |
| Ex 3772 | 2%-4% | 8%-10% | 2%-4% | 6%-8% | 4%-6% |
| Ex 3773 | 2%-4% | 8%-10% | 2%-4% | 6%-8% | 6%-8% |
| Ex 3774 | 2%-4% | 8%-10% | 2%-4% | 6%-8% | 8%-10% |
| Ex 3775 | 2%-4% | 8%-10% | 2%-4% | 8%-10% | 0%-1% |
| Ex 3776 | 2%-4% | 8%-10% | 2%-4% | 8%-10% | 1%-2% |
| Ex 3777 | 2%-4% | 8%-10% | 2%-4% | 8%-10% | 2%-4% |
| Ex 3778 | 2%-4% | 8%-10% | 2%-4% | 8%-10% | 4%-6% |
| Ex 3779 | 2%-4% | 8%-10% | 2%-4% | 8%-10% | 6%-8% |
| Ex 3780 | 2%-4% | 8%-10% | 2%-4% | 8%-10% | 8%-10% |
| Ex 3781 | 2%-4% | 8%-10% | 4%-6% | 0%-1% | 0%-1% |
| Ex 3782 | 2%-4% | 8%-10% | 4%-6% | 0%-1% | 1%-2% |
| Ex 3783 | 2%-4% | 8%-10% | 4%-6% | 0%-1% | 2%-4% |
| Ex 3784 | 2%-4% | 8%-10% | 4%-6% | 0%-1% | 4%-6% |
| Ex 3785 | 2%-4% | 8%-10% | 4%-6% | 0%-1% | 6%-8% |
| Ex 3786 | 2%-4% | 8%-10% | 4%-6% | 0%-1% | 8%-10% |
| Ex 3787 | 2%-4% | 8%-10% | 4%-6% | 1%-2% | 0%-1% |
| Ex 3788 | 2%-4% | 8%-10% | 4%-6% | 1%-2% | 1%-2% |
| Ex 3789 | 2%-4% | 8%-10% | 4%-6% | 1%-2% | 2%-4% |
| Ex 3790 | 2%-4% | 8%-10% | 4%-6% | 1%-2% | 4%-6% |
| Ex 3791 | 2%-4% | 8%-10% | 4%-6% | 1%-2% | 6%-8% |
| Ex 3792 | 2%-4% | 8%-10% | 4%-6% | 1%-2% | 8%-10% |
| Ex 3793 | 2%-4% | 8%-10% | 4%-6% | 2%-4% | 0%-1% |
| Ex 3794 | 2%-4% | 8%-10% | 4%-6% | 2%-4% | 1%-2% |
| Ex 3795 | 2%-4% | 8%-10% | 4%-6% | 2%-4% | 2%-4% |
| Ex 3796 | 2%-4% | 8%-10% | 4%-6% | 2%-4% | 4%-6% |
| Ex 3797 | 2%-4% | 8%-10% | 4%-6% | 2%-4% | 6%-8% |
| Ex 3798 | 2%-4% | 8%-10% | 4%-6% | 2%-4% | 8%-10% |
| Ex 3799 | 2%-4% | 8%-10% | 4%-6% | 4%-6% | 0%-1% |
| Ex 3800 | 2%-4% | 8%-10% | 4%-6% | 4%-6% | 1%-2% |
| Ex 3801 | 2%-4% | 8%-10% | 4%-6% | 4%-6% | 2%-4% |
| Ex 3802 | 2%-4% | 8%-10% | 4%-6% | 4%-6% | 4%-6% |
| Ex 3803 | 2%-4% | 8%-10% | 4%-6% | 4%-6% | 6%-8% |
| Ex 3804 | 2%-4% | 8%-10% | 4%-6% | 4%-6% | 8%-10% |
| Ex 3805 | 2%-4% | 8%-10% | 4%-6% | 6%-8% | 0%-1% |
| Ex 3806 | 2%-4% | 8%-10% | 4%-6% | 6%-8% | 1%-2% |
| Ex 3807 | 2%-4% | 8%-10% | 4%-6% | 6%-8% | 2%-4% |
| Ex 3808 | 2%-4% | 8%-10% | 4%-6% | 6%-8% | 4%-6% |
| Ex 3809 | 2%-4% | 8%-10% | 4%-6% | 6%-8% | 6%-8% |
| Ex 3810 | 2%-4% | 8%-10% | 4%-6% | 6%-8% | 8%-10% |
| Ex 3811 | 2%-4% | 8%-10% | 4%-6% | 8%-10% | 0%-1% |
| Ex 3812 | 2%-4% | 8%-10% | 4%-6% | 8%-10% | 1%-2% |
| Ex 3813 | 2%-4% | 8%-10% | 4%-6% | 8%-10% | 2%-4% |
| Ex 3814 | 2%-4% | 8%-10% | 4%-6% | 8%-10% | 4%-6% |
| Ex 3815 | 2%-4% | 8%-10% | 4%-6% | 8%-10% | 6%-8% |
| Ex 3816 | 2%-4% | 8%-10% | 4%-6% | 8%-10% | 8%-10% |
| Ex 3817 | 2%-4% | 8%-10% | 6%-8% | 0%-1% | 0%-1% |
| Ex 3818 | 2%-4% | 8%-10% | 6%-8% | 0%-1% | 1%-2% |
| Ex 3819 | 2%-4% | 8%-10% | 6%-8% | 0%-1% | 2%-4% |
| Ex 3820 | 2%-4% | 8%-10% | 6%-8% | 0%-1% | 4%-6% |
| Ex 3821 | 2%-4% | 8%-10% | 6%-8% | 0%-1% | 6%-8% |
| Ex 3822 | 2%-4% | 8%-10% | 6%-8% | 0%-1% | 8%-10% |
| Ex 3823 | 2%-4% | 8%-10% | 6%-8% | 1%-2% | 0%-1% |
| Ex 3824 | 2%-4% | 8%-10% | 6%-8% | 1%-2% | 1%-2% |
| Ex 3825 | 2%-4% | 8%-10% | 6%-8% | 1%-2% | 2%-4% |
| Ex 3826 | 2%-4% | 8%-10% | 6%-8% | 1%-2% | 4%-6% |
| Ex 3827 | 2%-4% | 8%-10% | 6%-8% | 1%-2% | 6%-8% |
| Ex 3828 | 2%-4% | 8%-10% | 6%-8% | 1%-2% | 8%-10% |
| Ex 3829 | 2%-4% | 8%-10% | 6%-8% | 2%-4% | 0%-1% |
| Ex 3830 | 2%-4% | 8%-10% | 6%-8% | 2%-4% | 1%-2% |
| Ex 3831 | 2%-4% | 8%-10% | 6%-8% | 2%-4% | 2%-4% |
| Ex 3832 | 2%-4% | 8%-10% | 6%-8% | 2%-4% | 4%-6% |
| Ex 3833 | 2%-4% | 8%-10% | 6%-8% | 2%-4% | 6%-8% |
| Ex 3834 | 2%-4% | 8%-10% | 6%-8% | 2%-4% | 8%-10% |
| Ex 3835 | 2%-4% | 8%-10% | 6%-8% | 4%-6% | 0%-1% |
| Ex 3836 | 2%-4% | 8%-10% | 6%-8% | 4%-6% | 1%-2% |
| Ex 3837 | 2%-4% | 8%-10% | 6%-8% | 4%-6% | 2%-4% |
| Ex 3838 | 2%-4% | 8%-10% | 6%-8% | 4%-6% | 4%-6% |
| Ex 3839 | 2%-4% | 8%-10% | 6%-8% | 4%-6% | 6%-8% |
| Ex 3840 | 2%-4% | 8%-10% | 6%-8% | 4%-6% | 8%-10% |
| Ex 3841 | 2%-4% | 8%-10% | 6%-8% | 6%-8% | 0%-1% |
| Ex 3842 | 2%-4% | 8%-10% | 6%-8% | 6%-8% | 1%-2% |
| Ex 3843 | 2%-4% | 8%-10% | 6%-8% | 6%-8% | 2%-4% |
| Ex 3844 | 2%-4% | 8%-10% | 6%-8% | 6%-8% | 4%-6% |
| Ex 3845 | 2%-4% | 8%-10% | 6%-8% | 6%-8% | 6%-8% |
| Ex 3846 | 2%-4% | 8%-10% | 6%-8% | 6%-8% | 8%-10% |
| Ex 3847 | 2%-4% | 8%-10% | 6%-8% | 8%-10% | 0%-1% |
| Ex 3848 | 2%-4% | 8%-10% | 6%-8% | 8%-10% | 1%-2% |
| Ex 3849 | 2%-4% | 8%-10% | 6%-8% | 8%-10% | 2%-4% |
| Ex 3850 | 2%-4% | 8%-10% | 6%-8% | 8%-10% | 4%-6% |
| Ex 3851 | 2%-4% | 8%-10% | 6%-8% | 8%-10% | 6%-8% |
| Ex 3852 | 2%-4% | 8%-10% | 6%-8% | 8%-10% | 8%-10% |
| Ex 3853 | 2%-4% | 8%-10% | 8%-10% | 0%-1% | 0%-1% |
| Ex 3854 | 2%-4% | 8%-10% | 8%-10% | 0%-1% | 1%-2% |
| Ex 3855 | 2%-4% | 8%-10% | 8%-10% | 0%-1% | 2%-4% |
| Ex 3856 | 2%-4% | 8%-10% | 8%-10% | 0%-1% | 4%-6% |
| Ex 3857 | 2%-4% | 8%-10% | 8%-10% | 0%-1% | 6%-8% |
| Ex 3858 | 2%-4% | 8%-10% | 8%-10% | 0%-1% | 8%-10% |
| Ex 3859 | 2%-4% | 8%-10% | 8%-10% | 1%-2% | 0%-1% |
| Ex 3860 | 2%-4% | 8%-10% | 8%-10% | 1%-2% | 1%-2% |
| Ex 3861 | 2%-4% | 8%-10% | 8%-10% | 1%-2% | 2%-4% |
| Ex 3862 | 2%-4% | 8%-10% | 8%-10% | 1%-2% | 4%-6% |
| Ex 3863 | 2%-4% | 8%-10% | 8%-10% | 1%-2% | 6%-8% |
| Ex 3864 | 2%-4% | 8%-10% | 8%-10% | 1%-2% | 8%-10% |
| Ex 3865 | 2%-4% | 8%-10% | 8%-10% | 2%-4% | 0%-1% |
| Ex 3866 | 2%-4% | 8%-10% | 8%-10% | 2%-4% | 1%-2% |
| Ex 3867 | 2%-4% | 8%-10% | 8%-10% | 2%-4% | 2%-4% |
| Ex 3868 | 2%-4% | 8%-10% | 8%-10% | 2%-4% | 4%-6% |
| Ex 3869 | 2%-4% | 8%-10% | 8%-10% | 2%-4% | 6%-8% |
| Ex 3870 | 2%-4% | 8%-10% | 8%-10% | 2%-4% | 8%-10% |
| Ex 3871 | 2%-4% | 8%-10% | 8%-10% | 4%-6% | 0%-1% |
| Ex 3872 | 2%-4% | 8%-10% | 8%-10% | 4%-6% | 1%-2% |
| Ex 3873 | 2%-4% | 8%-10% | 8%-10% | 4%-6% | 2%-4% |
| Ex 3874 | 2%-4% | 8%-10% | 8%-10% | 4%-6% | 4%-6% |
| Ex 3875 | 2%-4% | 8%-10% | 8%-10% | 4%-6% | 6%-8% |
| Ex 3876 | 2%-4% | 8%-10% | 8%-10% | 4%-6% | 8%-10% |
| Ex 3877 | 2%-4% | 8%-10% | 8%-10% | 6%-8% | 0%-1% |
| Ex 3878 | 2%-4% | 8%-10% | 8%-10% | 6%-8% | 1%-2% |
| Ex 3879 | 2%-4% | 8%-10% | 8%-10% | 6%-8% | 2%-4% |
| Ex 3880 | 2%-4% | 8%-10% | 8%-10% | 6%-8% | 4%-6% |
| Ex 3881 | 2%-4% | 8%-10% | 8%-10% | 6%-8% | 6%-8% |
| Ex 3882 | 2%-4% | 8%-10% | 8%-10% | 6%-8% | 8%-10% |
| Ex 3883 | 2%-4% | 8%-10% | 8%-10% | 8%-10% | 0%-1% |
| Ex 3884 | 2%-4% | 8%-10% | 8%-10% | 8%-10% | 1%-2% |
| Ex 3885 | 2%-4% | 8%-10% | 8%-10% | 8%-10% | 2%-4% |
| Ex 3886 | 2%-4% | 8%-10% | 8%-10% | 8%-10% | 4%-6% |
| Ex 3887 | 2%-4% | 8%-10% | 8%-10% | 8%-10% | 6%-8% |
| Ex 3888 | 2%-4% | 8%-10% | 8%-10% | 8%-10% | 8%-10% |
| Ex 3889 | 4%-6% | 0%-1% | 0%-1% | 0%-1% | 0%-1% |
| Ex 3890 | 4%-6% | 0%-1% | 0%-1% | 0%-1% | 1%-2% |
| Ex 3891 | 4%-6% | 0%-1% | 0%-1% | 0%-1% | 2%-4% |
| Ex 3892 | 4%-6% | 0%-1% | 0%-1% | 0%-1% | 4%-6% |
| Ex 3893 | 4%-6% | 0%-1% | 0%-1% | 0%-1% | 6%-8% |
| Ex 3894 | 4%-6% | 0%-1% | 0%-1% | 0%-1% | 8%-10% |
| Ex 3895 | 4%-6% | 0%-1% | 0%-1% | 1%-2% | 0%-1% |
| Ex 3896 | 4%-6% | 0%-1% | 0%-1% | 1%-2% | 1%-2% |
| Ex 3897 | 4%-6% | 0%-1% | 0%-1% | 1%-2% | 2%-4% |
| Ex 3898 | 4%-6% | 0%-1% | 0%-1% | 1%-2% | 4%-6% |
| Ex 3899 | 4%-6% | 0%-1% | 0%-1% | 1%-2% | 6%-8% |
| Ex 3900 | 4%-6% | 0%-1% | 0%-1% | 1%-2% | 8%-10% |
| Ex 3901 | 4%-6% | 0%-1% | 0%-1% | 2%-4% | 0%-1% |
| Ex 3902 | 4%-6% | 0%-1% | 0%-1% | 2%-4% | 1%-2% |
| Ex 3903 | 4%-6% | 0%-1% | 0%-1% | 2%-4% | 2%-4% |
| Ex 3904 | 4%-6% | 0%-1% | 0%-1% | 2%-4% | 4%-6% |
| Ex 3905 | 4%-6% | 0%-1% | 0%-1% | 2%-4% | 6%-8% |
| Ex 3906 | 4%-6% | 0%-1% | 0%-1% | 2%-4% | 8%-10% |
| Ex 3907 | 4%-6% | 0%-1% | 0%-1% | 4%-6% | 0%-1% |
| Ex 3908 | 4%-6% | 0%-1% | 0%-1% | 4%-6% | 1%-2% |
| Ex 3909 | 4%-6% | 0%-1% | 0%-1% | 4%-6% | 2%-4% |
| Ex 3910 | 4%-6% | 0%-1% | 0%-1% | 4%-6% | 4%-6% |
| Ex 3911 | 4%-6% | 0%-1% | 0%-1% | 4%-6% | 6%-8% |
| Ex 3912 | 4%-6% | 0%-1% | 0%-1% | 4%-6% | 8%-10% |
| Ex 3913 | 4%-6% | 0%-1% | 0%-1% | 6%-8% | 0%-1% |
| Ex 3914 | 4%-6% | 0%-1% | 0%-1% | 6%-8% | 1%-2% |
| Ex 3915 | 4%-6% | 0%-1% | 0%-1% | 6%-8% | 2%-4% |
| Ex 3916 | 4%-6% | 0%-1% | 0%-1% | 6%-8% | 4%-6% |
| Ex 3917 | 4%-6% | 0%-1% | 0%-1% | 6%-8% | 6%-8% |
| Ex 3918 | 4%-6% | 0%-1% | 0%-1% | 6%-8% | 8%-10% |
| Ex 3919 | 4%-6% | 0%-1% | 0%-1% | 8%-10% | 0%-1% |
| Ex 3920 | 4%-6% | 0%-1% | 0%-1% | 8%-10% | 1%-2% |
| Ex 3921 | 4%-6% | 0%-1% | 0%-1% | 8%-10% | 2%-4% |
| Ex 3922 | 4%-6% | 0%-1% | 0%-1% | 8%-10% | 4%-6% |
| Ex 3923 | 4%-6% | 0%-1% | 0%-1% | 8%-10% | 6%-8% |
| Ex 3924 | 4%-6% | 0%-1% | 0%-1% | 8%-10% | 8%-10% |
| Ex 3925 | 4%-6% | 0%-1% | 1%-2% | 0%-1% | 0%-1% |
| Ex 3926 | 4%-6% | 0%-1% | 1%-2% | 0%-1% | 1%-2% |
| Ex 3927 | 4%-6% | 0%-1% | 1%-2% | 0%-1% | 2%-4% |
| Ex 3928 | 4%-6% | 0%-1% | 1%-2% | 0%-1% | 4%-6% |
| Ex 3929 | 4%-6% | 0%-1% | 1%-2% | 0%-1% | 6%-8% |
| Ex 3930 | 4%-6% | 0%-1% | 1%-2% | 0%-1% | 8%-10% |
| Ex 3931 | 4%-6% | 0%-1% | 1%-2% | 1%-2% | 0%-1% |
| Ex 3932 | 4%-6% | 0%-1% | 1%-2% | 1%-2% | 1%-2% |
| Ex 3933 | 4%-6% | 0%-1% | 1%-2% | 1%-2% | 2%-4% |
| Ex 3934 | 4%-6% | 0%-1% | 1%-2% | 1%-2% | 4%-6% |
| Ex 3935 | 4%-6% | 0%-1% | 1%-2% | 1%-2% | 6%-8% |
| Ex 3936 | 4%-6% | 0%-1% | 1%-2% | 1%-2% | 8%-10% |
| Ex 3937 | 4%-6% | 0%-1% | 1%-2% | 2%-4% | 0%-1% |
| Ex 3938 | 4%-6% | 0%-1% | 1%-2% | 2%-4% | 1%-2% |
| Ex 3939 | 4%-6% | 0%-1% | 1%-2% | 2%-4% | 2%-4% |
| Ex 3940 | 4%-6% | 0%-1% | 1%-2% | 2%-4% | 4%-6% |
| Ex 3941 | 4%-6% | 0%-1% | 1%-2% | 2%-4% | 6%-8% |
| Ex 3942 | 4%-6% | 0%-1% | 1%-2% | 2%-4% | 8%-10% |
| Ex 3943 | 4%-6% | 0%-1% | 1%-2% | 4%-6% | 0%-1% |
| Ex 3944 | 4%-6% | 0%-1% | 1%-2% | 4%-6% | 1%-2% |
| Ex 3945 | 4%-6% | 0%-1% | 1%-2% | 4%-6% | 2%-4% |
| Ex 3946 | 4%-6% | 0%-1% | 1%-2% | 4%-6% | 4%-6% |
| Ex 3947 | 4%-6% | 0%-1% | 1%-2% | 4%-6% | 6%-8% |
| Ex 3948 | 4%-6% | 0%-1% | 1%-2% | 4%-6% | 8%-10% |
| Ex 3949 | 4%-6% | 0%-1% | 1%-2% | 6%-8% | 0%-1% |
| Ex 3950 | 4%-6% | 0%-1% | 1%-2% | 6%-8% | 1%-2% |
| Ex 3951 | 4%-6% | 0%-1% | 1%-2% | 6%-8% | 2%-4% |
| Ex 3952 | 4%-6% | 0%-1% | 1%-2% | 6%-8% | 4%-6% |
| Ex 3953 | 4%-6% | 0%-1% | 1%-2% | 6%-8% | 6%-8% |
| Ex 3954 | 4%-6% | 0%-1% | 1%-2% | 6%-8% | 8%-10% |
| Ex 3955 | 4%-6% | 0%-1% | 1%-2% | 8%-10% | 0%-1% |
| Ex 3956 | 4%-6% | 0%-1% | 1%-2% | 8%-10% | 1%-2% |
| Ex 3957 | 4%-6% | 0%-1% | 1%-2% | 8%-10% | 2%-4% |
| Ex 3958 | 4%-6% | 0%-1% | 1%-2% | 8%-10% | 4%-6% |
| Ex 3959 | 4%-6% | 0%-1% | 1%-2% | 8%-10% | 6%-8% |
| Ex 3960 | 4%-6% | 0%-1% | 1%-2% | 8%-10% | 8%-10% |
| Ex 3961 | 4%-6% | 0%-1% | 2%-4% | 0%-1% | 0%-1% |
| Ex 3962 | 4%-6% | 0%-1% | 2%-4% | 0%-1% | 1%-2% |
| Ex 3963 | 4%-6% | 0%-1% | 2%-4% | 0%-1% | 2%-4% |
| Ex 3964 | 4%-6% | 0%-1% | 2%-4% | 0%-1% | 4%-6% |
| Ex 3965 | 4%-6% | 0%-1% | 2%-4% | 0%-1% | 6%-8% |
| Ex 3966 | 4%-6% | 0%-1% | 2%-4% | 0%-1% | 8%-10% |
| Ex 3967 | 4%-6% | 0%-1% | 2%-4% | 1%-2% | 0%-1% |
| Ex 3968 | 4%-6% | 0%-1% | 2%-4% | 1%-2% | 1%-2% |
| Ex 3969 | 4%-6% | 0%-1% | 2%-4% | 1%-2% | 2%-4% |
| Ex 3970 | 4%-6% | 0%-1% | 2%-4% | 1%-2% | 4%-6% |
| Ex 3971 | 4%-6% | 0%-1% | 2%-4% | 1%-2% | 6%-8% |
| Ex 3972 | 4%-6% | 0%-1% | 2%-4% | 1%-2% | 8%-10% |
| Ex 3973 | 4%-6% | 0%-1% | 2%-4% | 2%-4% | 0%-1% |
| Ex 3974 | 4%-6% | 0%-1% | 2%-4% | 2%-4% | 1%-2% |
| Ex 3975 | 4%-6% | 0%-1% | 2%-4% | 2%-4% | 2%-4% |
| Ex 3976 | 4%-6% | 0%-1% | 2%-4% | 2%-4% | 4%-6% |
| Ex 3977 | 4%-6% | 0%-1% | 2%-4% | 2%-4% | 6%-8% |
| Ex 3978 | 4%-6% | 0%-1% | 2%-4% | 2%-4% | 8%-10% |
| Ex 3979 | 4%-6% | 0%-1% | 2%-4% | 4%-6% | 0%-1% |
| Ex 3980 | 4%-6% | 0%-1% | 2%-4% | 4%-6% | 1%-2% |
| Ex 3981 | 4%-6% | 0%-1% | 2%-4% | 4%-6% | 2%-4% |
| Ex 3982 | 4%-6% | 0%-1% | 2%-4% | 4%-6% | 4%-6% |
| Ex 3983 | 4%-6% | 0%-1% | 2%-4% | 4%-6% | 6%-8% |
| Ex 3984 | 4%-6% | 0%-1% | 2%-4% | 4%-6% | 8%-10% |
| Ex 3985 | 4%-6% | 0%-1% | 2%-4% | 6%-8% | 0%-1% |
| Ex 3986 | 4%-6% | 0%-1% | 2%-4% | 6%-8% | 1%-2% |
| Ex 3987 | 4%-6% | 0%-1% | 2%-4% | 6%-8% | 2%-4% |
| Ex 3988 | 4%-6% | 0%-1% | 2%-4% | 6%-8% | 4%-6% |
| Ex 3989 | 4%-6% | 0%-1% | 2%-4% | 6%-8% | 6%-8% |
| Ex 3990 | 4%-6% | 0%-1% | 2%-4% | 6%-8% | 8%-10% |
| Ex 3991 | 4%-6% | 0%-1% | 2%-4% | 8%-10% | 0%-1% |
| Ex 3992 | 4%-6% | 0%-1% | 2%-4% | 8%-10% | 1%-2% |
| Ex 3993 | 4%-6% | 0%-1% | 2%-4% | 8%-10% | 2%-4% |
| Ex 3994 | 4%-6% | 0%-1% | 2%-4% | 8%-10% | 4%-6% |
| Ex 3995 | 4%-6% | 0%-1% | 2%-4% | 8%-10% | 6%-8% |
| Ex 3996 | 4%-6% | 0%-1% | 2%-4% | 8%-10% | 8%-10% |
| Ex 3997 | 4%-6% | 0%-1% | 4%-6% | 0%-1% | 0%-1% |
| Ex 3998 | 4%-6% | 0%-1% | 4%-6% | 0%-1% | 1%-2% |
| Ex 3999 | 4%-6% | 0%-1% | 4%-6% | 0%-1% | 2%-4% |
| Ex 4000 | 4%-6% | 0%-1% | 4%-6% | 0%-1% | 4%-6% |
| Ex 4001 | 4%-6% | 0%-1% | 4%-6% | 0%-1% | 6%-8% |
| Ex 4002 | 4%-6% | 0%-1% | 4%-6% | 0%-1% | 8%-10% |
| Ex 4003 | 4%-6% | 0%-1% | 4%-6% | 1%-2% | 0%-1% |
| Ex 4004 | 4%-6% | 0%-1% | 4%-6% | 1%-2% | 1%-2% |
| Ex 4005 | 4%-6% | 0%-1% | 4%-6% | 1%-2% | 2%-4% |
| Ex 4006 | 4%-6% | 0%-1% | 4%-6% | 1%-2% | 4%-6% |
| Ex 4007 | 4%-6% | 0%-1% | 4%-6% | 1%-2% | 6%-8% |
| Ex 4008 | 4%-6% | 0%-1% | 4%-6% | 1%-2% | 8%-10% |
| Ex 4009 | 4%-6% | 0%-1% | 4%-6% | 2%-4% | 0%-1% |
| Ex 4010 | 4%-6% | 0%-1% | 4%-6% | 2%-4% | 1%-2% |
| Ex 4011 | 4%-6% | 0%-1% | 4%-6% | 2%-4% | 2%-4% |
| Ex 4012 | 4%-6% | 0%-1% | 4%-6% | 2%-4% | 4%-6% |
| Ex 4013 | 4%-6% | 0%-1% | 4%-6% | 2%-4% | 6%-8% |
| Ex 4014 | 4%-6% | 0%-1% | 4%-6% | 2%-4% | 8%-10% |
| Ex 4015 | 4%-6% | 0%-1% | 4%-6% | 4%-6% | 0%-1% |
| Ex 4016 | 4%-6% | 0%-1% | 4%-6% | 4%-6% | 1%-2% |
| Ex 4017 | 4%-6% | 0%-1% | 4%-6% | 4%-6% | 2%-4% |
| Ex 4018 | 4%-6% | 0%-1% | 4%-6% | 4%-6% | 4%-6% |
| Ex 4019 | 4%-6% | 0%-1% | 4%-6% | 4%-6% | 6%-8% |
| Ex 4020 | 4%-6% | 0%-1% | 4%-6% | 4%-6% | 8%-10% |
| Ex 4021 | 4%-6% | 0%-1% | 4%-6% | 6%-8% | 0%-1% |
| Ex 4022 | 4%-6% | 0%-1% | 4%-6% | 6%-8% | 1%-2% |
| Ex 4023 | 4%-6% | 0%-1% | 4%-6% | 6%-8% | 2%-4% |
| Ex 4024 | 4%-6% | 0%-1% | 4%-6% | 6%-8% | 4%-6% |
| Ex 4025 | 4%-6% | 0%-1% | 4%-6% | 6%-8% | 6%-8% |
| Ex 4026 | 4%-6% | 0%-1% | 4%-6% | 6%-8% | 8%-10% |
| Ex 4027 | 4%-6% | 0%-1% | 4%-6% | 8%-10% | 0%-1% |
| Ex 4028 | 4%-6% | 0%-1% | 4%-6% | 8%-10% | 1%-2% |
| Ex 4029 | 4%-6% | 0%-1% | 4%-6% | 8%-10% | 2%-4% |
| Ex 4030 | 4%-6% | 0%-1% | 4%-6% | 8%-10% | 4%-6% |
| Ex 4031 | 4%-6% | 0%-1% | 4%-6% | 8%-10% | 6%-8% |
| Ex 4032 | 4%-6% | 0%-1% | 4%-6% | 8%-10% | 8%-10% |
| Ex 4033 | 4%-6% | 0%-1% | 6%-8% | 0%-1% | 0%-1% |
| Ex 4034 | 4%-6% | 0%-1% | 6%-8% | 0%-1% | 1%-2% |
| Ex 4035 | 4%-6% | 0%-1% | 6%-8% | 0%-1% | 2%-4% |
| Ex 4036 | 4%-6% | 0%-1% | 6%-8% | 0%-1% | 4%-6% |
| Ex 4037 | 4%-6% | 0%-1% | 6%-8% | 0%-1% | 6%-8% |
| Ex 4038 | 4%-6% | 0%-1% | 6%-8% | 0%-1% | 8%-10% |
| Ex 4039 | 4%-6% | 0%-1% | 6%-8% | 1%-2% | 0%-1% |
| Ex 4040 | 4%-6% | 0%-1% | 6%-8% | 1%-2% | 1%-2% |
| Ex 4041 | 4%-6% | 0%-1% | 6%-8% | 1%-2% | 2%-4% |
| Ex 4042 | 4%-6% | 0%-1% | 6%-8% | 1%-2% | 4%-6% |
| Ex 4043 | 4%-6% | 0%-1% | 6%-8% | 1%-2% | 6%-8% |
| Ex 4044 | 4%-6% | 0%-1% | 6%-8% | 1%-2% | 8%-10% |
| Ex 4045 | 4%-6% | 0%-1% | 6%-8% | 2%-4% | 0%-1% |
| Ex 4046 | 4%-6% | 0%-1% | 6%-8% | 2%-4% | 1%-2% |
| Ex 4047 | 4%-6% | 0%-1% | 6%-8% | 2%-4% | 2%-4% |
| Ex 4048 | 4%-6% | 0%-1% | 6%-8% | 2%-4% | 4%-6% |
| Ex 4049 | 4%-6% | 0%-1% | 6%-8% | 2%-4% | 6%-8% |
| Ex 4050 | 4%-6% | 0%-1% | 6%-8% | 2%-4% | 8%-10% |
| Ex 4051 | 4%-6% | 0%-1% | 6%-8% | 4%-6% | 0%-1% |
| Ex 4052 | 4%-6% | 0%-1% | 6%-8% | 4%-6% | 1%-2% |
| Ex 4053 | 4%-6% | 0%-1% | 6%-8% | 4%-6% | 2%-4% |
| Ex 4054 | 4%-6% | 0%-1% | 6%-8% | 4%-6% | 4%-6% |
| Ex 4055 | 4%-6% | 0%-1% | 6%-8% | 4%-6% | 6%-8% |
| Ex 4056 | 4%-6% | 0%-1% | 6%-8% | 4%-6% | 8%-10% |
| Ex 4057 | 4%-6% | 0%-1% | 6%-8% | 6%-8% | 0%-1% |
| Ex 4058 | 4%-6% | 0%-1% | 6%-8% | 6%-8% | 1%-2% |
| Ex 4059 | 4%-6% | 0%-1% | 6%-8% | 6%-8% | 2%-4% |
| Ex 4060 | 4%-6% | 0%-1% | 6%-8% | 6%-8% | 4%-6% |
| Ex 4061 | 4%-6% | 0%-1% | 6%-8% | 6%-8% | 6%-8% |
| Ex 4062 | 4%-6% | 0%-1% | 6%-8% | 6%-8% | 8%-10% |
| Ex 4063 | 4%-6% | 0%-1% | 6%-8% | 8%-10% | 0%-1% |
| Ex 4064 | 4%-6% | 0%-1% | 6%-8% | 8%-10% | 1%-2% |
| Ex 4065 | 4%-6% | 0%-1% | 6%-8% | 8%-10% | 2%-4% |
| Ex 4066 | 4%-6% | 0%-1% | 6%-8% | 8%-10% | 4%-6% |
| Ex 4067 | 4%-6% | 0%-1% | 6%-8% | 8%-10% | 6%-8% |
| Ex 4068 | 4%-6% | 0%-1% | 6%-8% | 8%-10% | 8%-10% |
| Ex 4069 | 4%-6% | 0%-1% | 8%-10% | 0%-1% | 0%-1% |
| Ex 4070 | 4%-6% | 0%-1% | 8%-10% | 0%-1% | 1%-2% |
| Ex 4071 | 4%-6% | 0%-1% | 8%-10% | 0%-1% | 2%-4% |
| Ex 4072 | 4%-6% | 0%-1% | 8%-10% | 0%-1% | 4%-6% |
| Ex 4073 | 4%-6% | 0%-1% | 8%-10% | 0%-1% | 6%-8% |
| Ex 4074 | 4%-6% | 0%-1% | 8%-10% | 0%-1% | 8%-10% |
| Ex 4075 | 4%-6% | 0%-1% | 8%-10% | 1%-2% | 0%-1% |
| Ex 4076 | 4%-6% | 0%-1% | 8%-10% | 1%-2% | 1%-2% |
| Ex 4077 | 4%-6% | 0%-1% | 8%-10% | 1%-2% | 2%-4% |
| Ex 4078 | 4%-6% | 0%-1% | 8%-10% | 1%-2% | 4%-6% |
| Ex 4079 | 4%-6% | 0%-1% | 8%-10% | 1%-2% | 6%-8% |
| Ex 4080 | 4%-6% | 0%-1% | 8%-10% | 1%-2% | 8%-10% |
| Ex 4081 | 4%-6% | 0%-1% | 8%-10% | 2%-4% | 0%-1% |
| Ex 4082 | 4%-6% | 0%-1% | 8%-10% | 2%-4% | 1%-2% |
| Ex 4083 | 4%-6% | 0%-1% | 8%-10% | 2%-4% | 2%-4% |
| Ex 4084 | 4%-6% | 0%-1% | 8%-10% | 2%-4% | 4%-6% |
| Ex 4085 | 4%-6% | 0%-1% | 8%-10% | 2%-4% | 6%-8% |
| Ex 4086 | 4%-6% | 0%-1% | 8%-10% | 2%-4% | 8%-10% |
| Ex 4087 | 4%-6% | 0%-1% | 8%-10% | 4%-6% | 0%-1% |
| Ex 4088 | 4%-6% | 0%-1% | 8%-10% | 4%-6% | 1%-2% |
| Ex 4089 | 4%-6% | 0%-1% | 8%-10% | 4%-6% | 2%-4% |
| Ex 4090 | 4%-6% | 0%-1% | 8%-10% | 4%-6% | 4%-6% |
| Ex 4091 | 4%-6% | 0%-1% | 8%-10% | 4%-6% | 6%-8% |
| Ex 4092 | 4%-6% | 0%-1% | 8%-10% | 4%-6% | 8%-10% |
| Ex 4093 | 4%-6% | 0%-1% | 8%-10% | 6%-8% | 0%-1% |
| Ex 4094 | 4%-6% | 0%-1% | 8%-10% | 6%-8% | 1%-2% |
| Ex 4095 | 4%-6% | 0%-1% | 8%-10% | 6%-8% | 2%-4% |
| Ex 4096 | 4%-6% | 0%-1% | 8%-10% | 6%-8% | 4%-6% |
| Ex 4097 | 4%-6% | 0%-1% | 8%-10% | 6%-8% | 6%-8% |
| Ex 4098 | 4%-6% | 0%-1% | 8%-10% | 6%-8% | 8%-10% |
| Ex 4099 | 4%-6% | 0%-1% | 8%-10% | 8%-10% | 0%-1% |
| Ex 4100 | 4%-6% | 0%-1% | 8%-10% | 8%-10% | 1%-2% |
| Ex 4101 | 4%-6% | 0%-1% | 8%-10% | 8%-10% | 2%-4% |
| Ex 4102 | 4%-6% | 0%-1% | 8%-10% | 8%-10% | 4%-6% |
| Ex 4103 | 4%-6% | 0%-1% | 8%-10% | 8%-10% | 6%-8% |
| Ex 4104 | 4%-6% | 0%-1% | 8%-10% | 8%-10% | 8%-10% |
| Ex 4105 | 4%-6% | 1%-2% | 0%-1% | 0%-1% | 0%-1% |
| Ex 4106 | 4%-6% | 1%-2% | 0%-1% | 0%-1% | 1%-2% |
| Ex 4107 | 4%-6% | 1%-2% | 0%-1% | 0%-1% | 2%-4% |
| Ex 4108 | 4%-6% | 1%-2% | 0%-1% | 0%-1% | 4%-6% |
| Ex 4109 | 4%-6% | 1%-2% | 0%-1% | 0%-1% | 6%-8% |
| Ex 4110 | 4%-6% | 1%-2% | 0%-1% | 0%-1% | 8%-10% |
| Ex 4111 | 4%-6% | 1%-2% | 0%-1% | 1%-2% | 0%-1% |
| Ex 4112 | 4%-6% | 1%-2% | 0%-1% | 1%-2% | 1%-2% |
| Ex 4113 | 4%-6% | 1%-2% | 0%-1% | 1%-2% | 2%-4% |
| Ex 4114 | 4%-6% | 1%-2% | 0%-1% | 1%-2% | 4%-6% |
| Ex 4115 | 4%-6% | 1%-2% | 0%-1% | 1%-2% | 6%-8% |
| Ex 4116 | 4%-6% | 1%-2% | 0%-1% | 1%-2% | 8%-10% |
| Ex 4117 | 4%-6% | 1%-2% | 0%-1% | 2%-4% | 0%-1% |
| Ex 4118 | 4%-6% | 1%-2% | 0%-1% | 2%-4% | 1%-2% |
| Ex 4119 | 4%-6% | 1%-2% | 0%-1% | 2%-4% | 2%-4% |
| Ex 4120 | 4%-6% | 1%-2% | 0%-1% | 2%-4% | 4%-6% |
| Ex 4121 | 4%-6% | 1%-2% | 0%-1% | 2%-4% | 6%-8% |
| Ex 4122 | 4%-6% | 1%-2% | 0%-1% | 2%-4% | 8%-10% |
| Ex 4123 | 4%-6% | 1%-2% | 0%-1% | 4%-6% | 0%-1% |
| Ex 4124 | 4%-6% | 1%-2% | 0%-1% | 4%-6% | 1%-2% |
| Ex 4125 | 4%-6% | 1%-2% | 0%-1% | 4%-6% | 2%-4% |
| Ex 4126 | 4%-6% | 1%-2% | 0%-1% | 4%-6% | 4%-6% |
| Ex 4127 | 4%-6% | 1%-2% | 0%-1% | 4%-6% | 6%-8% |
| Ex 4128 | 4%-6% | 1%-2% | 0%-1% | 4%-6% | 8%-10% |
| Ex 4129 | 4%-6% | 1%-2% | 0%-1% | 6%-8% | 0%-1% |
| Ex 4130 | 4%-6% | 1%-2% | 0%-1% | 6%-8% | 1%-2% |
| Ex 4131 | 4%-6% | 1%-2% | 0%-1% | 6%-8% | 2%-4% |
| Ex 4132 | 4%-6% | 1%-2% | 0%-1% | 6%-8% | 4%-6% |
| Ex 4133 | 4%-6% | 1%-2% | 0%-1% | 6%-8% | 6%-8% |
| Ex 4134 | 4%-6% | 1%-2% | 0%-1% | 6%-8% | 8%-10% |
| Ex 4135 | 4%-6% | 1%-2% | 0%-1% | 8%-10% | 0%-1% |
| Ex 4136 | 4%-6% | 1%-2% | 0%-1% | 8%-10% | 1%-2% |
| Ex 4137 | 4%-6% | 1%-2% | 0%-1% | 8%-10% | 2%-4% |
| Ex 4138 | 4%-6% | 1%-2% | 0%-1% | 8%-10% | 4%-6% |
| Ex 4139 | 4%-6% | 1%-2% | 0%-1% | 8%-10% | 6%-8% |
| Ex 4140 | 4%-6% | 1%-2% | 0%-1% | 8%-10% | 8%-10% |
| Ex 4141 | 4%-6% | 1%-2% | 1%-2% | 0%-1% | 0%-1% |
| Ex 4142 | 4%-6% | 1%-2% | 1%-2% | 0%-1% | 1%-2% |
| Ex 4143 | 4%-6% | 1%-2% | 1%-2% | 0%-1% | 2%-4% |
| Ex 4144 | 4%-6% | 1%-2% | 1%-2% | 0%-1% | 4%-6% |
| Ex 4145 | 4%-6% | 1%-2% | 1%-2% | 0%-1% | 6%-8% |
| Ex 4146 | 4%-6% | 1%-2% | 1%-2% | 0%-1% | 8%-10% |
| Ex 4147 | 4%-6% | 1%-2% | 1%-2% | 1%-2% | 0%-1% |
| Ex 4148 | 4%-6% | 1%-2% | 1%-2% | 1%-2% | 1%-2% |
| Ex 4149 | 4%-6% | 1%-2% | 1%-2% | 1%-2% | 2%-4% |
| Ex 4150 | 4%-6% | 1%-2% | 1%-2% | 1%-2% | 4%-6% |
| Ex 4151 | 4%-6% | 1%-2% | 1%-2% | 1%-2% | 6%-8% |
| Ex 4152 | 4%-6% | 1%-2% | 1%-2% | 1%-2% | 8%-10% |
| Ex 4153 | 4%-6% | 1%-2% | 1%-2% | 2%-4% | 0%-1% |
| Ex 4154 | 4%-6% | 1%-2% | 1%-2% | 2%-4% | 1%-2% |
| Ex 4155 | 4%-6% | 1%-2% | 1%-2% | 2%-4% | 2%-4% |
| Ex 4156 | 4%-6% | 1%-2% | 1%-2% | 2%-4% | 4%-6% |
| Ex 4157 | 4%-6% | 1%-2% | 1%-2% | 2%-4% | 6%-8% |
| Ex 4158 | 4%-6% | 1%-2% | 1%-2% | 2%-4% | 8%-10% |
| Ex 4159 | 4%-6% | 1%-2% | 1%-2% | 4%-6% | 0%-1% |
| Ex 4160 | 4%-6% | 1%-2% | 1%-2% | 4%-6% | 1%-2% |
| Ex 4161 | 4%-6% | 1%-2% | 1%-2% | 4%-6% | 2%-4% |
| Ex 4162 | 4%-6% | 1%-2% | 1%-2% | 4%-6% | 4%-6% |
| Ex 4163 | 4%-6% | 1%-2% | 1%-2% | 4%-6% | 6%-8% |
| Ex 4164 | 4%-6% | 1%-2% | 1%-2% | 4%-6% | 8%-10% |
| Ex 4165 | 4%-6% | 1%-2% | 1%-2% | 6%-8% | 0%-1% |
| Ex 4166 | 4%-6% | 1%-2% | 1%-2% | 6%-8% | 1%-2% |
| Ex 4167 | 4%-6% | 1%-2% | 1%-2% | 6%-8% | 2%-4% |
| Ex 4168 | 4%-6% | 1%-2% | 1%-2% | 6%-8% | 4%-6% |
| Ex 4169 | 4%-6% | 1%-2% | 1%-2% | 6%-8% | 6%-8% |
| Ex 4170 | 4%-6% | 1%-2% | 1%-2% | 6%-8% | 8%-10% |
| Ex 4171 | 4%-6% | 1%-2% | 1%-2% | 8%-10% | 0%-1% |
| Ex 4172 | 4%-6% | 1%-2% | 1%-2% | 8%-10% | 1%-2% |
| Ex 4173 | 4%-6% | 1%-2% | 1%-2% | 8%-10% | 2%-4% |
| Ex 4174 | 4%-6% | 1%-2% | 1%-2% | 8%-10% | 4%-6% |
| Ex 4175 | 4%-6% | 1%-2% | 1%-2% | 8%-10% | 6%-8% |
| Ex 4176 | 4%-6% | 1%-2% | 1%-2% | 8%-10% | 8%-10% |
| Ex 4177 | 4%-6% | 1%-2% | 2%-4% | 0%-1% | 0%-1% |
| Ex 4178 | 4%-6% | 1%-2% | 2%-4% | 0%-1% | 1%-2% |
| Ex 4179 | 4%-6% | 1%-2% | 2%-4% | 0%-1% | 2%-4% |
| Ex 4180 | 4%-6% | 1%-2% | 2%-4% | 0%-1% | 4%-6% |
| Ex 4181 | 4%-6% | 1%-2% | 2%-4% | 0%-1% | 6%-8% |
| Ex 4182 | 4%-6% | 1%-2% | 2%-4% | 0%-1% | 8%-10% |
| Ex 4183 | 4%-6% | 1%-2% | 2%-4% | 1%-2% | 0%-1% |
| Ex 4184 | 4%-6% | 1%-2% | 2%-4% | 1%-2% | 1%-2% |
| Ex 4185 | 4%-6% | 1%-2% | 2%-4% | 1%-2% | 2%-4% |
| Ex 4186 | 4%-6% | 1%-2% | 2%-4% | 1%-2% | 4%-6% |
| Ex 4187 | 4%-6% | 1%-2% | 2%-4% | 1%-2% | 6%-8% |
| Ex 4188 | 4%-6% | 1%-2% | 2%-4% | 1%-2% | 8%-10% |
| Ex 4189 | 4%-6% | 1%-2% | 2%-4% | 2%-4% | 0%-1% |
| Ex 4190 | 4%-6% | 1%-2% | 2%-4% | 2%-4% | 1%-2% |
| Ex 4191 | 4%-6% | 1%-2% | 2%-4% | 2%-4% | 2%-4% |
| Ex 4192 | 4%-6% | 1%-2% | 2%-4% | 2%-4% | 4%-6% |
| Ex 4193 | 4%-6% | 1%-2% | 2%-4% | 2%-4% | 6%-8% |
| Ex 4194 | 4%-6% | 1%-2% | 2%-4% | 2%-4% | 8%-10% |
| Ex 4195 | 4%-6% | 1%-2% | 2%-4% | 4%-6% | 0%-1% |
| Ex 4196 | 4%-6% | 1%-2% | 2%-4% | 4%-6% | 1%-2% |
| Ex 4197 | 4%-6% | 1%-2% | 2%-4% | 4%-6% | 2%-4% |
| Ex 4198 | 4%-6% | 1%-2% | 2%-4% | 4%-6% | 4%-6% |
| Ex 4199 | 4%-6% | 1%-2% | 2%-4% | 4%-6% | 6%-8% |
| Ex 4200 | 4%-6% | 1%-2% | 2%-4% | 4%-6% | 8%-10% |
| Ex 4201 | 4%-6% | 1%-2% | 2%-4% | 6%-8% | 0%-1% |
| Ex 4202 | 4%-6% | 1%-2% | 2%-4% | 6%-8% | 1%-2% |
| Ex 4203 | 4%-6% | 1%-2% | 2%-4% | 6%-8% | 2%-4% |
| Ex 4204 | 4%-6% | 1%-2% | 2%-4% | 6%-8% | 4%-6% |
| Ex 4205 | 4%-6% | 1%-2% | 2%-4% | 6%-8% | 6%-8% |
| Ex 4206 | 4%-6% | 1%-2% | 2%-4% | 6%-8% | 8%-10% |
| Ex 4207 | 4%-6% | 1%-2% | 2%-4% | 8%-10% | 0%-1% |
| Ex 4208 | 4%-6% | 1%-2% | 2%-4% | 8%-10% | 1%-2% |
| Ex 4209 | 4%-6% | 1%-2% | 2%-4% | 8%-10% | 2%-4% |
| Ex 4210 | 4%-6% | 1%-2% | 2%-4% | 8%-10% | 4%-6% |
| Ex 4211 | 4%-6% | 1%-2% | 2%-4% | 8%-10% | 6%-8% |
| Ex 4212 | 4%-6% | 1%-2% | 2%-4% | 8%-10% | 8%-10% |
| Ex 4213 | 4%-6% | 1%-2% | 4%-6% | 0%-1% | 0%-1% |
| Ex 4214 | 4%-6% | 1%-2% | 4%-6% | 0%-1% | 1%-2% |
| Ex 4215 | 4%-6% | 1%-2% | 4%-6% | 0%-1% | 2%-4% |
| Ex 4216 | 4%-6% | 1%-2% | 4%-6% | 0%-1% | 4%-6% |
| Ex 4217 | 4%-6% | 1%-2% | 4%-6% | 0%-1% | 6%-8% |
| Ex 4218 | 4%-6% | 1%-2% | 4%-6% | 0%-1% | 8%-10% |
| Ex 4219 | 4%-6% | 1%-2% | 4%-6% | 1%-2% | 0%-1% |
| Ex 4220 | 4%-6% | 1%-2% | 4%-6% | 1%-2% | 1%-2% |
| Ex 4221 | 4%-6% | 1%-2% | 4%-6% | 1%-2% | 2%-4% |
| Ex 4222 | 4%-6% | 1%-2% | 4%-6% | 1%-2% | 4%-6% |
| Ex 4223 | 4%-6% | 1%-2% | 4%-6% | 1%-2% | 6%-8% |
| Ex 4224 | 4%-6% | 1%-2% | 4%-6% | 1%-2% | 8%-10% |
| Ex 4225 | 4%-6% | 1%-2% | 4%-6% | 2%-4% | 0%-1% |
| Ex 4226 | 4%-6% | 1%-2% | 4%-6% | 2%-4% | 1%-2% |
| Ex 4227 | 4%-6% | 1%-2% | 4%-6% | 2%-4% | 2%-4% |
| Ex 4228 | 4%-6% | 1%-2% | 4%-6% | 2%-4% | 4%-6% |
| Ex 4229 | 4%-6% | 1%-2% | 4%-6% | 2%-4% | 6%-8% |
| Ex 4230 | 4%-6% | 1%-2% | 4%-6% | 2%-4% | 8%-10% |
| Ex 4231 | 4%-6% | 1%-2% | 4%-6% | 4%-6% | 0%-1% |
| Ex 4232 | 4%-6% | 1%-2% | 4%-6% | 4%-6% | 1%-2% |
| Ex 4233 | 4%-6% | 1%-2% | 4%-6% | 4%-6% | 2%-4% |
| Ex 4234 | 4%-6% | 1%-2% | 4%-6% | 4%-6% | 4%-6% |
| Ex 4235 | 4%-6% | 1%-2% | 4%-6% | 4%-6% | 6%-8% |
| Ex 4236 | 4%-6% | 1%-2% | 4%-6% | 4%-6% | 8%-10% |
| Ex 4237 | 4%-6% | 1%-2% | 4%-6% | 6%-8% | 0%-1% |
| Ex 4238 | 4%-6% | 1%-2% | 4%-6% | 6%-8% | 1%-2% |
| Ex 4239 | 4%-6% | 1%-2% | 4%-6% | 6%-8% | 2%-4% |
| Ex 4240 | 4%-6% | 1%-2% | 4%-6% | 6%-8% | 4%-6% |
| Ex 4241 | 4%-6% | 1%-2% | 4%-6% | 6%-8% | 6%-8% |
| Ex 4242 | 4%-6% | 1%-2% | 4%-6% | 6%-8% | 8%-10% |
| Ex 4243 | 4%-6% | 1%-2% | 4%-6% | 8%-10% | 0%-1% |
| Ex 4244 | 4%-6% | 1%-2% | 4%-6% | 8%-10% | 1%-2% |
| Ex 4245 | 4%-6% | 1%-2% | 4%-6% | 8%-10% | 2%-4% |
| Ex 4246 | 4%-6% | 1%-2% | 4%-6% | 8%-10% | 4%-6% |
| Ex 4247 | 4%-6% | 1%-2% | 4%-6% | 8%-10% | 6%-8% |
| Ex 4248 | 4%-6% | 1%-2% | 4%-6% | 8%-10% | 8%-10% |
| Ex 4249 | 4%-6% | 1%-2% | 6%-8% | 0%-1% | 0%-1% |
| Ex 4250 | 4%-6% | 1%-2% | 6%-8% | 0%-1% | 1%-2% |
| Ex 4251 | 4%-6% | 1%-2% | 6%-8% | 0%-1% | 2%-4% |
| Ex 4252 | 4%-6% | 1%-2% | 6%-8% | 0%-1% | 4%-6% |
| Ex 4253 | 4%-6% | 1%-2% | 6%-8% | 0%-1% | 6%-8% |
| Ex 4254 | 4%-6% | 1%-2% | 6%-8% | 0%-1% | 8%-10% |
| Ex 4255 | 4%-6% | 1%-2% | 6%-8% | 1%-2% | 0%-1% |
| Ex 4256 | 4%-6% | 1%-2% | 6%-8% | 1%-2% | 1%-2% |
| Ex 4257 | 4%-6% | 1%-2% | 6%-8% | 1%-2% | 2%-4% |
| Ex 4258 | 4%-6% | 1%-2% | 6%-8% | 1%-2% | 4%-6% |
| Ex 4259 | 4%-6% | 1%-2% | 6%-8% | 1%-2% | 6%-8% |
| Ex 4260 | 4%-6% | 1%-2% | 6%-8% | 1%-2% | 8%-10% |
| Ex 4261 | 4%-6% | 1%-2% | 6%-8% | 2%-4% | 0%-1% |
| Ex 4262 | 4%-6% | 1%-2% | 6%-8% | 2%-4% | 1%-2% |
| Ex 4263 | 4%-6% | 1%-2% | 6%-8% | 2%-4% | 2%-4% |
| Ex 4264 | 4%-6% | 1%-2% | 6%-8% | 2%-4% | 4%-6% |
| Ex 4265 | 4%-6% | 1%-2% | 6%-8% | 2%-4% | 6%-8% |
| Ex 4266 | 4%-6% | 1%-2% | 6%-8% | 2%-4% | 8%-10% |
| Ex 4267 | 4%-6% | 1%-2% | 6%-8% | 4%-6% | 0%-1% |
| Ex 4268 | 4%-6% | 1%-2% | 6%-8% | 4%-6% | 1%-2% |
| Ex 4269 | 4%-6% | 1%-2% | 6%-8% | 4%-6% | 2%-4% |
| Ex 4270 | 4%-6% | 1%-2% | 6%-8% | 4%-6% | 4%-6% |
| Ex 4271 | 4%-6% | 1%-2% | 6%-8% | 4%-6% | 6%-8% |
| Ex 4272 | 4%-6% | 1%-2% | 6%-8% | 4%-6% | 8%-10% |
| Ex 4273 | 4%-6% | 1%-2% | 6%-8% | 6%-8% | 0%-1% |
| Ex 4274 | 4%-6% | 1%-2% | 6%-8% | 6%-8% | 1%-2% |
| Ex 4275 | 4%-6% | 1%-2% | 6%-8% | 6%-8% | 2%-4% |
| Ex 4276 | 4%-6% | 1%-2% | 6%-8% | 6%-8% | 4%-6% |
| Ex 4277 | 4%-6% | 1%-2% | 6%-8% | 6%-8% | 6%-8% |
| Ex 4278 | 4%-6% | 1%-2% | 6%-8% | 6%-8% | 8%-10% |
| Ex 4279 | 4%-6% | 1%-2% | 6%-8% | 8%-10% | 0%-1% |
| Ex 4280 | 4%-6% | 1%-2% | 6%-8% | 8%-10% | 1%-2% |
| Ex 4281 | 4%-6% | 1%-2% | 6%-8% | 8%-10% | 2%-4% |
| Ex 4282 | 4%-6% | 1%-2% | 6%-8% | 8%-10% | 4%-6% |
| Ex 4283 | 4%-6% | 1%-2% | 6%-8% | 8%-10% | 6%-8% |
| Ex 4284 | 4%-6% | 1%-2% | 6%-8% | 8%-10% | 8%-10% |
| Ex 4285 | 4%-6% | 1%-2% | 8%-10% | 0%-1% | 0%-1% |
| Ex 4286 | 4%-6% | 1%-2% | 8%-10% | 0%-1% | 1%-2% |
| Ex 4287 | 4%-6% | 1%-2% | 8%-10% | 0%-1% | 2%-4% |
| Ex 4288 | 4%-6% | 1%-2% | 8%-10% | 0%-1% | 4%-6% |
| Ex 4289 | 4%-6% | 1%-2% | 8%-10% | 0%-1% | 6%-8% |
| Ex 4290 | 4%-6% | 1%-2% | 8%-10% | 0%-1% | 8%-10% |
| Ex 4291 | 4%-6% | 1%-2% | 8%-10% | 1%-2% | 0%-1% |
| Ex 4292 | 4%-6% | 1%-2% | 8%-10% | 1%-2% | 1%-2% |
| Ex 4293 | 4%-6% | 1%-2% | 8%-10% | 1%-2% | 2%-4% |
| Ex 4294 | 4%-6% | 1%-2% | 8%-10% | 1%-2% | 4%-6% |
| Ex 4295 | 4%-6% | 1%-2% | 8%-10% | 1%-2% | 6%-8% |
| Ex 4296 | 4%-6% | 1%-2% | 8%-10% | 1%-2% | 8%-10% |
| Ex 4297 | 4%-6% | 1%-2% | 8%-10% | 2%-4% | 0%-1% |
| Ex 4298 | 4%-6% | 1%-2% | 8%-10% | 2%-4% | 1%-2% |
| Ex 4299 | 4%-6% | 1%-2% | 8%-10% | 2%-4% | 2%-4% |
| Ex 4300 | 4%-6% | 1%-2% | 8%-10% | 2%-4% | 4%-6% |
| Ex 4301 | 4%-6% | 1%-2% | 8%-10% | 2%-4% | 6%-8% |
| Ex 4302 | 4%-6% | 1%-2% | 8%-10% | 2%-4% | 8%-10% |
| Ex 4303 | 4%-6% | 1%-2% | 8%-10% | 4%-6% | 0%-1% |
| Ex 4304 | 4%-6% | 1%-2% | 8%-10% | 4%-6% | 1%-2% |
| Ex 4305 | 4%-6% | 1%-2% | 8%-10% | 4%-6% | 2%-4% |
| Ex 4306 | 4%-6% | 1%-2% | 8%-10% | 4%-6% | 4%-6% |
| Ex 4307 | 4%-6% | 1%-2% | 8%-10% | 4%-6% | 6%-8% |
| Ex 4308 | 4%-6% | 1%-2% | 8%-10% | 4%-6% | 8%-10% |
| Ex 4309 | 4%-6% | 1%-2% | 8%-10% | 6%-8% | 0%-1% |
| Ex 4310 | 4%-6% | 1%-2% | 8%-10% | 6%-8% | 1%-2% |
| Ex 4311 | 4%-6% | 1%-2% | 8%-10% | 6%-8% | 2%-4% |
| Ex 4312 | 4%-6% | 1%-2% | 8%-10% | 6%-8% | 4%-6% |
| Ex 4313 | 4%-6% | 1%-2% | 8%-10% | 6%-8% | 6%-8% |
| Ex 4314 | 4%-6% | 1%-2% | 8%-10% | 6%-8% | 8%-10% |
| Ex 4315 | 4%-6% | 1%-2% | 8%-10% | 8%-10% | 0%-1% |
| Ex 4316 | 4%-6% | 1%-2% | 8%-10% | 8%-10% | 1%-2% |
| Ex 4317 | 4%-6% | 1%-2% | 8%-10% | 8%-10% | 2%-4% |
| Ex 4318 | 4%-6% | 1%-2% | 8%-10% | 8%-10% | 4%-6% |
| Ex 4319 | 4%-6% | 1%-2% | 8%-10% | 8%-10% | 6%-8% |
| Ex 4320 | 4%-6% | 1%-2% | 8%-10% | 8%-10% | 8%-10% |
| Ex 4321 | 4%-6% | 2%-4% | 0%-1% | 0%-1% | 0%-1% |
| Ex 4322 | 4%-6% | 2%-4% | 0%-1% | 0%-1% | 1%-2% |
| Ex 4323 | 4%-6% | 2%-4% | 0%-1% | 0%-1% | 2%-4% |
| Ex 4324 | 4%-6% | 2%-4% | 0%-1% | 0%-1% | 4%-6% |
| Ex 4325 | 4%-6% | 2%-4% | 0%-1% | 0%-1% | 6%-8% |
| Ex 4326 | 4%-6% | 2%-4% | 0%-1% | 0%-1% | 8%-10% |
| Ex 4327 | 4%-6% | 2%-4% | 0%-1% | 1%-2% | 0%-1% |
| Ex 4328 | 4%-6% | 2%-4% | 0%-1% | 1%-2% | 1%-2% |
| Ex 4329 | 4%-6% | 2%-4% | 0%-1% | 1%-2% | 2%-4% |
| Ex 4330 | 4%-6% | 2%-4% | 0%-1% | 1%-2% | 4%-6% |
| Ex 4331 | 4%-6% | 2%-4% | 0%-1% | 1%-2% | 6%-8% |
| Ex 4332 | 4%-6% | 2%-4% | 0%-1% | 1%-2% | 8%-10% |
| Ex 4333 | 4%-6% | 2%-4% | 0%-1% | 2%-4% | 0%-1% |
| Ex 4334 | 4%-6% | 2%-4% | 0%-1% | 2%-4% | 1%-2% |
| Ex 4335 | 4%-6% | 2%-4% | 0%-1% | 2%-4% | 2%-4% |
| Ex 4336 | 4%-6% | 2%-4% | 0%-1% | 2%-4% | 4%-6% |
| Ex 4337 | 4%-6% | 2%-4% | 0%-1% | 2%-4% | 6%-8% |
| Ex 4338 | 4%-6% | 2%-4% | 0%-1% | 2%-4% | 8%-10% |
| Ex 4339 | 4%-6% | 2%-4% | 0%-1% | 4%-6% | 0%-1% |
| Ex 4340 | 4%-6% | 2%-4% | 0%-1% | 4%-6% | 1%-2% |
| Ex 4341 | 4%-6% | 2%-4% | 0%-1% | 4%-6% | 2%-4% |
| Ex 4342 | 4%-6% | 2%-4% | 0%-1% | 4%-6% | 4%-6% |
| Ex 4343 | 4%-6% | 2%-4% | 0%-1% | 4%-6% | 6%-8% |
| Ex 4344 | 4%-6% | 2%-4% | 0%-1% | 4%-6% | 8%-10% |
| Ex 4345 | 4%-6% | 2%-4% | 0%-1% | 6%-8% | 0%-1% |
| Ex 4346 | 4%-6% | 2%-4% | 0%-1% | 6%-8% | 1%-2% |
| Ex 4347 | 4%-6% | 2%-4% | 0%-1% | 6%-8% | 2%-4% |
| Ex 4348 | 4%-6% | 2%-4% | 0%-1% | 6%-8% | 4%-6% |
| Ex 4349 | 4%-6% | 2%-4% | 0%-1% | 6%-8% | 6%-8% |
| Ex 4350 | 4%-6% | 2%-4% | 0%-1% | 6%-8% | 8%-10% |
| Ex 4351 | 4%-6% | 2%-4% | 0%-1% | 8%-10% | 0%-1% |
| Ex 4352 | 4%-6% | 2%-4% | 0%-1% | 8%-10% | 1%-2% |
| Ex 4353 | 4%-6% | 2%-4% | 0%-1% | 8%-10% | 2%-4% |
| Ex 4354 | 4%-6% | 2%-4% | 0%-1% | 8%-10% | 4%-6% |
| Ex 4355 | 4%-6% | 2%-4% | 0%-1% | 8%-10% | 6%-8% |
| Ex 4356 | 4%-6% | 2%-4% | 0%-1% | 8%-10% | 8%-10% |
| Ex 4357 | 4%-6% | 2%-4% | 1%-2% | 0%-1% | 0%-1% |
| Ex 4358 | 4%-6% | 2%-4% | 1%-2% | 0%-1% | 1%-2% |
| Ex 4359 | 4%-6% | 2%-4% | 1%-2% | 0%-1% | 2%-4% |
| Ex 4360 | 4%-6% | 2%-4% | 1%-2% | 0%-1% | 4%-6% |
| Ex 4361 | 4%-6% | 2%-4% | 1%-2% | 0%-1% | 6%-8% |
| Ex 4362 | 4%-6% | 2%-4% | 1%-2% | 0%-1% | 8%-10% |
| Ex 4363 | 4%-6% | 2%-4% | 1%-2% | 1%-2% | 0%-1% |
| Ex 4364 | 4%-6% | 2%-4% | 1%-2% | 1%-2% | 1%-2% |
| Ex 4365 | 4%-6% | 2%-4% | 1%-2% | 1%-2% | 2%-4% |
| Ex 4366 | 4%-6% | 2%-4% | 1%-2% | 1%-2% | 4%-6% |
| Ex 4367 | 4%-6% | 2%-4% | 1%-2% | 1%-2% | 6%-8% |
| Ex 4368 | 4%-6% | 2%-4% | 1%-2% | 1%-2% | 8%-10% |
| Ex 4369 | 4%-6% | 2%-4% | 1%-2% | 2%-4% | 0%-1% |
| Ex 4370 | 4%-6% | 2%-4% | 1%-2% | 2%-4% | 1%-2% |
| Ex 4371 | 4%-6% | 2%-4% | 1%-2% | 2%-4% | 2%-4% |
| Ex 4372 | 4%-6% | 2%-4% | 1%-2% | 2%-4% | 4%-6% |
| Ex 4373 | 4%-6% | 2%-4% | 1%-2% | 2%-4% | 6%-8% |
| Ex 4374 | 4%-6% | 2%-4% | 1%-2% | 2%-4% | 8%-10% |
| Ex 4375 | 4%-6% | 2%-4% | 1%-2% | 4%-6% | 0%-1% |
| Ex 4376 | 4%-6% | 2%-4% | 1%-2% | 4%-6% | 1%-2% |
| Ex 4377 | 4%-6% | 2%-4% | 1%-2% | 4%-6% | 2%-4% |
| Ex 4378 | 4%-6% | 2%-4% | 1%-2% | 4%-6% | 4%-6% |
| Ex 4379 | 4%-6% | 2%-4% | 1%-2% | 4%-6% | 6%-8% |
| Ex 4380 | 4%-6% | 2%-4% | 1%-2% | 4%-6% | 8%-10% |
| Ex 4381 | 4%-6% | 2%-4% | 1%-2% | 6%-8% | 0%-1% |
| Ex 4382 | 4%-6% | 2%-4% | 1%-2% | 6%-8% | 1%-2% |
| Ex 4383 | 4%-6% | 2%-4% | 1%-2% | 6%-8% | 2%-4% |
| Ex 4384 | 4%-6% | 2%-4% | 1%-2% | 6%-8% | 4%-6% |
| Ex 4385 | 4%-6% | 2%-4% | 1%-2% | 6%-8% | 6%-8% |
| Ex 4386 | 4%-6% | 2%-4% | 1%-2% | 6%-8% | 8%-10% |
| Ex 4387 | 4%-6% | 2%-4% | 1%-2% | 8%-10% | 0%-1% |
| Ex 4388 | 4%-6% | 2%-4% | 1%-2% | 8%-10% | 1%-2% |
| Ex 4389 | 4%-6% | 2%-4% | 1%-2% | 8%-10% | 2%-4% |
| Ex 4390 | 4%-6% | 2%-4% | 1%-2% | 8%-10% | 4%-6% |
| Ex 4391 | 4%-6% | 2%-4% | 1%-2% | 8%-10% | 6%-8% |
| Ex 4392 | 4%-6% | 2%-4% | 1%-2% | 8%-10% | 8%-10% |
| Ex 4393 | 4%-6% | 2%-4% | 2%-4% | 0%-1% | 0%-1% |
| Ex 4394 | 4%-6% | 2%-4% | 2%-4% | 0%-1% | 1%-2% |
| Ex 4395 | 4%-6% | 2%-4% | 2%-4% | 0%-1% | 2%-4% |
| Ex 4396 | 4%-6% | 2%-4% | 2%-4% | 0%-1% | 4%-6% |
| Ex 4397 | 4%-6% | 2%-4% | 2%-4% | 0%-1% | 6%-8% |
| Ex 4398 | 4%-6% | 2%-4% | 2%-4% | 0%-1% | 8%-10% |
| Ex 4399 | 4%-6% | 2%-4% | 2%-4% | 1%-2% | 0%-1% |
| Ex 4400 | 4%-6% | 2%-4% | 2%-4% | 1%-2% | 1%-2% |
| Ex 4401 | 4%-6% | 2%-4% | 2%-4% | 1%-2% | 2%-4% |
| Ex 4402 | 4%-6% | 2%-4% | 2%-4% | 1%-2% | 4%-6% |
| Ex 4403 | 4%-6% | 2%-4% | 2%-4% | 1%-2% | 6%-8% |
| Ex 4404 | 4%-6% | 2%-4% | 2%-4% | 1%-2% | 8%-10% |
| Ex 4405 | 4%-6% | 2%-4% | 2%-4% | 2%-4% | 0%-1% |
| Ex 4406 | 4%-6% | 2%-4% | 2%-4% | 2%-4% | 1%-2% |
| Ex 4407 | 4%-6% | 2%-4% | 2%-4% | 2%-4% | 2%-4% |
| Ex 4408 | 4%-6% | 2%-4% | 2%-4% | 2%-4% | 4%-6% |
| Ex 4409 | 4%-6% | 2%-4% | 2%-4% | 2%-4% | 6%-8% |
| Ex 4410 | 4%-6% | 2%-4% | 2%-4% | 2%-4% | 8%-10% |
| Ex 4411 | 4%-6% | 2%-4% | 2%-4% | 4%-6% | 0%-1% |
| Ex 4412 | 4%-6% | 2%-4% | 2%-4% | 4%-6% | 1%-2% |
| Ex 4413 | 4%-6% | 2%-4% | 2%-4% | 4%-6% | 2%-4% |
| Ex 4414 | 4%-6% | 2%-4% | 2%-4% | 4%-6% | 4%-6% |
| Ex 4415 | 4%-6% | 2%-4% | 2%-4% | 4%-6% | 6%-8% |
| Ex 4416 | 4%-6% | 2%-4% | 2%-4% | 4%-6% | 8%-10% |
| Ex 4417 | 4%-6% | 2%-4% | 2%-4% | 6%-8% | 0%-1% |
| Ex 4418 | 4%-6% | 2%-4% | 2%-4% | 6%-8% | 1%-2% |
| Ex 4419 | 4%-6% | 2%-4% | 2%-4% | 6%-8% | 2%-4% |
| Ex 4420 | 4%-6% | 2%-4% | 2%-4% | 6%-8% | 4%-6% |
| Ex 4421 | 4%-6% | 2%-4% | 2%-4% | 6%-8% | 6%-8% |
| Ex 4422 | 4%-6% | 2%-4% | 2%-4% | 6%-8% | 8%-10% |
| Ex 4423 | 4%-6% | 2%-4% | 2%-4% | 8%-10% | 0%-1% |
| Ex 4424 | 4%-6% | 2%-4% | 2%-4% | 8%-10% | 1%-2% |
| Ex 4425 | 4%-6% | 2%-4% | 2%-4% | 8%-10% | 2%-4% |
| Ex 4426 | 4%-6% | 2%-4% | 2%-4% | 8%-10% | 4%-6% |
| Ex 4427 | 4%-6% | 2%-4% | 2%-4% | 8%-10% | 6%-8% |
| Ex 4428 | 4%-6% | 2%-4% | 2%-4% | 8%-10% | 8%-10% |
| Ex 4429 | 4%-6% | 2%-4% | 4%-6% | 0%-1% | 0%-1% |
| Ex 4430 | 4%-6% | 2%-4% | 4%-6% | 0%-1% | 1%-2% |
| Ex 4431 | 4%-6% | 2%-4% | 4%-6% | 0%-1% | 2%-4% |
| Ex 4432 | 4%-6% | 2%-4% | 4%-6% | 0%-1% | 4%-6% |
| Ex 4433 | 4%-6% | 2%-4% | 4%-6% | 0%-1% | 6%-8% |
| Ex 4434 | 4%-6% | 2%-4% | 4%-6% | 0%-1% | 8%-10% |
| Ex 4435 | 4%-6% | 2%-4% | 4%-6% | 1%-2% | 0%-1% |
| Ex 4436 | 4%-6% | 2%-4% | 4%-6% | 1%-2% | 1%-2% |
| Ex 4437 | 4%-6% | 2%-4% | 4%-6% | 1%-2% | 2%-4% |
| Ex 4438 | 4%-6% | 2%-4% | 4%-6% | 1%-2% | 4%-6% |
| Ex 4439 | 4%-6% | 2%-4% | 4%-6% | 1%-2% | 6%-8% |
| Ex 4440 | 4%-6% | 2%-4% | 4%-6% | 1%-2% | 8%-10% |
| Ex 4441 | 4%-6% | 2%-4% | 4%-6% | 2%-4% | 0%-1% |
| Ex 4442 | 4%-6% | 2%-4% | 4%-6% | 2%-4% | 1%-2% |
| Ex 4443 | 4%-6% | 2%-4% | 4%-6% | 2%-4% | 2%-4% |
| Ex 4444 | 4%-6% | 2%-4% | 4%-6% | 2%-4% | 4%-6% |
| Ex 4445 | 4%-6% | 2%-4% | 4%-6% | 2%-4% | 6%-8% |
| Ex 4446 | 4%-6% | 2%-4% | 4%-6% | 2%-4% | 8%-10% |
| Ex 4447 | 4%-6% | 2%-4% | 4%-6% | 4%-6% | 0%-1% |
| Ex 4448 | 4%-6% | 2%-4% | 4%-6% | 4%-6% | 1%-2% |
| Ex 4449 | 4%-6% | 2%-4% | 4%-6% | 4%-6% | 2%-4% |
| Ex 4450 | 4%-6% | 2%-4% | 4%-6% | 4%-6% | 4%-6% |
| Ex 4451 | 4%-6% | 2%-4% | 4%-6% | 4%-6% | 6%-8% |
| Ex 4452 | 4%-6% | 2%-4% | 4%-6% | 4%-6% | 8%-10% |
| Ex 4453 | 4%-6% | 2%-4% | 4%-6% | 6%-8% | 0%-1% |
| Ex 4454 | 4%-6% | 2%-4% | 4%-6% | 6%-8% | 1%-2% |
| Ex 4455 | 4%-6% | 2%-4% | 4%-6% | 6%-8% | 2%-4% |
| Ex 4456 | 4%-6% | 2%-4% | 4%-6% | 6%-8% | 4%-6% |
| Ex 4457 | 4%-6% | 2%-4% | 4%-6% | 6%-8% | 6%-8% |
| Ex 4458 | 4%-6% | 2%-4% | 4%-6% | 6%-8% | 8%-10% |
| Ex 4459 | 4%-6% | 2%-4% | 4%-6% | 8%-10% | 0%-1% |
| Ex 4460 | 4%-6% | 2%-4% | 4%-6% | 8%-10% | 1%-2% |
| Ex 4461 | 4%-6% | 2%-4% | 4%-6% | 8%-10% | 2%-4% |
| Ex 4462 | 4%-6% | 2%-4% | 4%-6% | 8%-10% | 4%-6% |
| Ex 4463 | 4%-6% | 2%-4% | 4%-6% | 8%-10% | 6%-8% |
| Ex 4464 | 4%-6% | 2%-4% | 4%-6% | 8%-10% | 8%-10% |
| Ex 4465 | 4%-6% | 2%-4% | 6%-8% | 0%-1% | 0%-1% |
| Ex 4466 | 4%-6% | 2%-4% | 6%-8% | 0%-1% | 1%-2% |
| Ex 4467 | 4%-6% | 2%-4% | 6%-8% | 0%-1% | 2%-4% |
| Ex 4468 | 4%-6% | 2%-4% | 6%-8% | 0%-1% | 4%-6% |
| Ex 4469 | 4%-6% | 2%-4% | 6%-8% | 0%-1% | 6%-8% |
| Ex 4470 | 4%-6% | 2%-4% | 6%-8% | 0%-1% | 8%-10% |
| Ex 4471 | 4%-6% | 2%-4% | 6%-8% | 1%-2% | 0%-1% |
| Ex 4472 | 4%-6% | 2%-4% | 6%-8% | 1%-2% | 1%-2% |
| Ex 4473 | 4%-6% | 2%-4% | 6%-8% | 1%-2% | 2%-4% |
| Ex 4474 | 4%-6% | 2%-4% | 6%-8% | 1%-2% | 4%-6% |
| Ex 4475 | 4%-6% | 2%-4% | 6%-8% | 1%-2% | 6%-8% |
| Ex 4476 | 4%-6% | 2%-4% | 6%-8% | 1%-2% | 8%-10% |
| Ex 4477 | 4%-6% | 2%-4% | 6%-8% | 2%-4% | 0%-1% |
| Ex 4478 | 4%-6% | 2%-4% | 6%-8% | 2%-4% | 1%-2% |
| Ex 4479 | 4%-6% | 2%-4% | 6%-8% | 2%-4% | 2%-4% |
| Ex 4480 | 4%-6% | 2%-4% | 6%-8% | 2%-4% | 4%-6% |
| Ex 4481 | 4%-6% | 2%-4% | 6%-8% | 2%-4% | 6%-8% |
| Ex 4482 | 4%-6% | 2%-4% | 6%-8% | 2%-4% | 8%-10% |
| Ex 4483 | 4%-6% | 2%-4% | 6%-8% | 4%-6% | 0%-1% |
| Ex 4484 | 4%-6% | 2%-4% | 6%-8% | 4%-6% | 1%-2% |
| Ex 4485 | 4%-6% | 2%-4% | 6%-8% | 4%-6% | 2%-4% |
| Ex 4486 | 4%-6% | 2%-4% | 6%-8% | 4%-6% | 4%-6% |
| Ex 4487 | 4%-6% | 2%-4% | 6%-8% | 4%-6% | 6%-8% |
| Ex 4488 | 4%-6% | 2%-4% | 6%-8% | 4%-6% | 8%-10% |
| Ex 4489 | 4%-6% | 2%-4% | 6%-8% | 6%-8% | 0%-1% |
| Ex 4490 | 4%-6% | 2%-4% | 6%-8% | 6%-8% | 1%-2% |
| Ex 4491 | 4%-6% | 2%-4% | 6%-8% | 6%-8% | 2%-4% |
| Ex 4492 | 4%-6% | 2%-4% | 6%-8% | 6%-8% | 4%-6% |
| Ex 4493 | 4%-6% | 2%-4% | 6%-8% | 6%-8% | 6%-8% |
| Ex 4494 | 4%-6% | 2%-4% | 6%-8% | 6%-8% | 8%-10% |
| Ex 4495 | 4%-6% | 2%-4% | 6%-8% | 8%-10% | 0%-1% |
| Ex 4496 | 4%-6% | 2%-4% | 6%-8% | 8%-10% | 1%-2% |
| Ex 4497 | 4%-6% | 2%-4% | 6%-8% | 8%-10% | 2%-4% |
| Ex 4498 | 4%-6% | 2%-4% | 6%-8% | 8%-10% | 4%-6% |
| Ex 4499 | 4%-6% | 2%-4% | 6%-8% | 8%-10% | 6%-8% |
| Ex 4500 | 4%-6% | 2%-4% | 6%-8% | 8%-10% | 8%-10% |
| Ex 4501 | 4%-6% | 2%-4% | 8%-10% | 0%-1% | 0%-1% |
| Ex 4502 | 4%-6% | 2%-4% | 8%-10% | 0%-1% | 1%-2% |
| Ex 4503 | 4%-6% | 2%-4% | 8%-10% | 0%-1% | 2%-4% |
| Ex 4504 | 4%-6% | 2%-4% | 8%-10% | 0%-1% | 4%-6% |
| Ex 4505 | 4%-6% | 2%-4% | 8%-10% | 0%-1% | 6%-8% |
| Ex 4506 | 4%-6% | 2%-4% | 8%-10% | 0%-1% | 8%-10% |
| Ex 4507 | 4%-6% | 2%-4% | 8%-10% | 1%-2% | 0%-1% |
| Ex 4508 | 4%-6% | 2%-4% | 8%-10% | 1%-2% | 1%-2% |
| Ex 4509 | 4%-6% | 2%-4% | 8%-10% | 1%-2% | 2%-4% |
| Ex 4510 | 4%-6% | 2%-4% | 8%-10% | 1%-2% | 4%-6% |
| Ex 4511 | 4%-6% | 2%-4% | 8%-10% | 1%-2% | 6%-8% |
| Ex 4512 | 4%-6% | 2%-4% | 8%-10% | 1%-2% | 8%-10% |
| Ex 4513 | 4%-6% | 2%-4% | 8%-10% | 2%-4% | 0%-1% |
| Ex 4514 | 4%-6% | 2%-4% | 8%-10% | 2%-4% | 1%-2% |
| Ex 4515 | 4%-6% | 2%-4% | 8%-10% | 2%-4% | 2%-4% |
| Ex 4516 | 4%-6% | 2%-4% | 8%-10% | 2%-4% | 4%-6% |
| Ex 4517 | 4%-6% | 2%-4% | 8%-10% | 2%-4% | 6%-8% |
| Ex 4518 | 4%-6% | 2%-4% | 8%-10% | 2%-4% | 8%-10% |
| Ex 4519 | 4%-6% | 2%-4% | 8%-10% | 4%-6% | 0%-1% |
| Ex 4520 | 4%-6% | 2%-4% | 8%-10% | 4%-6% | 1%-2% |
| Ex 4521 | 4%-6% | 2%-4% | 8%-10% | 4%-6% | 2%-4% |
| Ex 4522 | 4%-6% | 2%-4% | 8%-10% | 4%-6% | 4%-6% |
| Ex 4523 | 4%-6% | 2%-4% | 8%-10% | 4%-6% | 6%-8% |
| Ex 4524 | 4%-6% | 2%-4% | 8%-10% | 4%-6% | 8%-10% |
| Ex 4525 | 4%-6% | 2%-4% | 8%-10% | 6%-8% | 0%-1% |
| Ex 4526 | 4%-6% | 2%-4% | 8%-10% | 6%-8% | 1%-2% |
| Ex 4527 | 4%-6% | 2%-4% | 8%-10% | 6%-8% | 2%-4% |
| Ex 4528 | 4%-6% | 2%-4% | 8%-10% | 6%-8% | 4%-6% |
| Ex 4529 | 4%-6% | 2%-4% | 8%-10% | 6%-8% | 6%-8% |
| Ex 4530 | 4%-6% | 2%-4% | 8%-10% | 6%-8% | 8%-10% |
| Ex 4531 | 4%-6% | 2%-4% | 8%-10% | 8%-10% | 0%-1% |
| Ex 4532 | 4%-6% | 2%-4% | 8%-10% | 8%-10% | 1%-2% |
| Ex 4533 | 4%-6% | 2%-4% | 8%-10% | 8%-10% | 2%-4% |
| Ex 4534 | 4%-6% | 2%-4% | 8%-10% | 8%-10% | 4%-6% |
| Ex 4535 | 4%-6% | 2%-4% | 8%-10% | 8%-10% | 6%-8% |
| Ex 4536 | 4%-6% | 2%-4% | 8%-10% | 8%-10% | 8%-10% |
| Ex 4537 | 4%-6% | 4%-6% | 0%-1% | 0%-1% | 0%-1% |
| Ex 4538 | 4%-6% | 4%-6% | 0%-1% | 0%-1% | 1%-2% |
| Ex 4539 | 4%-6% | 4%-6% | 0%-1% | 0%-1% | 2%-4% |
| Ex 4540 | 4%-6% | 4%-6% | 0%-1% | 0%-1% | 4%-6% |
| Ex 4541 | 4%-6% | 4%-6% | 0%-1% | 0%-1% | 6%-8% |
| Ex 4542 | 4%-6% | 4%-6% | 0%-1% | 0%-1% | 8%-10% |
| Ex 4543 | 4%-6% | 4%-6% | 0%-1% | 1%-2% | 0%-1% |
| Ex 4544 | 4%-6% | 4%-6% | 0%-1% | 1%-2% | 1%-2% |
| Ex 4545 | 4%-6% | 4%-6% | 0%-1% | 1%-2% | 2%-4% |
| Ex 4546 | 4%-6% | 4%-6% | 0%-1% | 1%-2% | 4%-6% |
| Ex 4547 | 4%-6% | 4%-6% | 0%-1% | 1%-2% | 6%-8% |
| Ex 4548 | 4%-6% | 4%-6% | 0%-1% | 1%-2% | 8%-10% |
| Ex 4549 | 4%-6% | 4%-6% | 0%-1% | 2%-4% | 0%-1% |
| Ex 4550 | 4%-6% | 4%-6% | 0%-1% | 2%-4% | 1%-2% |
| Ex 4551 | 4%-6% | 4%-6% | 0%-1% | 2%-4% | 2%-4% |
| Ex 4552 | 4%-6% | 4%-6% | 0%-1% | 2%-4% | 4%-6% |
| Ex 4553 | 4%-6% | 4%-6% | 0%-1% | 2%-4% | 6%-8% |
| Ex 4554 | 4%-6% | 4%-6% | 0%-1% | 2%-4% | 8%-10% |
| Ex 4555 | 4%-6% | 4%-6% | 0%-1% | 4%-6% | 0%-1% |
| Ex 4556 | 4%-6% | 4%-6% | 0%-1% | 4%-6% | 1%-2% |
| Ex 4557 | 4%-6% | 4%-6% | 0%-1% | 4%-6% | 2%-4% |
| Ex 4558 | 4%-6% | 4%-6% | 0%-1% | 4%-6% | 4%-6% |
| Ex 4559 | 4%-6% | 4%-6% | 0%-1% | 4%-6% | 6%-8% |
| Ex 4560 | 4%-6% | 4%-6% | 0%-1% | 4%-6% | 8%-10% |
| Ex 4561 | 4%-6% | 4%-6% | 0%-1% | 6%-8% | 0%-1% |
| Ex 4562 | 4%-6% | 4%-6% | 0%-1% | 6%-8% | 1%-2% |
| Ex 4563 | 4%-6% | 4%-6% | 0%-1% | 6%-8% | 2%-4% |
| Ex 4564 | 4%-6% | 4%-6% | 0%-1% | 6%-8% | 4%-6% |
| Ex 4565 | 4%-6% | 4%-6% | 0%-1% | 6%-8% | 6%-8% |
| Ex 4566 | 4%-6% | 4%-6% | 0%-1% | 6%-8% | 8%-10% |
| Ex 4567 | 4%-6% | 4%-6% | 0%-1% | 8%-10% | 0%-1% |
| Ex 4568 | 4%-6% | 4%-6% | 0%-1% | 8%-10% | 1%-2% |
| Ex 4569 | 4%-6% | 4%-6% | 0%-1% | 8%-10% | 2%-4% |
| Ex 4570 | 4%-6% | 4%-6% | 0%-1% | 8%-10% | 4%-6% |
| Ex 4571 | 4%-6% | 4%-6% | 0%-1% | 8%-10% | 6%-8% |
| Ex 4572 | 4%-6% | 4%-6% | 0%-1% | 8%-10% | 8%-10% |
| Ex 4573 | 4%-6% | 4%-6% | 1%-2% | 0%-1% | 0%-1% |
| Ex 4574 | 4%-6% | 4%-6% | 1%-2% | 0%-1% | 1%-2% |
| Ex 4575 | 4%-6% | 4%-6% | 1%-2% | 0%-1% | 2%-4% |
| Ex 4576 | 4%-6% | 4%-6% | 1%-2% | 0%-1% | 4%-6% |
| Ex 4577 | 4%-6% | 4%-6% | 1%-2% | 0%-1% | 6%-8% |
| Ex 4578 | 4%-6% | 4%-6% | 1%-2% | 0%-1% | 8%-10% |
| Ex 4579 | 4%-6% | 4%-6% | 1%-2% | 1%-2% | 0%-1% |
| Ex 4580 | 4%-6% | 4%-6% | 1%-2% | 1%-2% | 1%-2% |
| Ex 4581 | 4%-6% | 4%-6% | 1%-2% | 1%-2% | 2%-4% |
| Ex 4582 | 4%-6% | 4%-6% | 1%-2% | 1%-2% | 4%-6% |
| Ex 4583 | 4%-6% | 4%-6% | 1%-2% | 1%-2% | 6%-8% |
| Ex 4584 | 4%-6% | 4%-6% | 1%-2% | 1%-2% | 8%-10% |
| Ex 4585 | 4%-6% | 4%-6% | 1%-2% | 2%-4% | 0%-1% |
| Ex 4586 | 4%-6% | 4%-6% | 1%-2% | 2%-4% | 1%-2% |
| Ex 4587 | 4%-6% | 4%-6% | 1%-2% | 2%-4% | 2%-4% |
| Ex 4588 | 4%-6% | 4%-6% | 1%-2% | 2%-4% | 4%-6% |
| Ex 4589 | 4%-6% | 4%-6% | 1%-2% | 2%-4% | 6%-8% |
| Ex 4590 | 4%-6% | 4%-6% | 1%-2% | 2%-4% | 8%-10% |
| Ex 4591 | 4%-6% | 4%-6% | 1%-2% | 4%-6% | 0%-1% |
| Ex 4592 | 4%-6% | 4%-6% | 1%-2% | 4%-6% | 1%-2% |
| Ex 4593 | 4%-6% | 4%-6% | 1%-2% | 4%-6% | 2%-4% |
| Ex 4594 | 4%-6% | 4%-6% | 1%-2% | 4%-6% | 4%-6% |
| Ex 4595 | 4%-6% | 4%-6% | 1%-2% | 4%-6% | 6%-8% |
| Ex 4596 | 4%-6% | 4%-6% | 1%-2% | 4%-6% | 8%-10% |
| Ex 4597 | 4%-6% | 4%-6% | 1%-2% | 6%-8% | 0%-1% |
| Ex 4598 | 4%-6% | 4%-6% | 1%-2% | 6%-8% | 1%-2% |
| Ex 4599 | 4%-6% | 4%-6% | 1%-2% | 6%-8% | 2%-4% |
| Ex 4600 | 4%-6% | 4%-6% | 1%-2% | 6%-8% | 4%-6% |
| Ex 4601 | 4%-6% | 4%-6% | 1%-2% | 6%-8% | 6%-8% |
| Ex 4602 | 4%-6% | 4%-6% | 1%-2% | 6%-8% | 8%-10% |
| Ex 4603 | 4%-6% | 4%-6% | 1%-2% | 8%-10% | 0%-1% |
| Ex 4604 | 4%-6% | 4%-6% | 1%-2% | 8%-10% | 1%-2% |
| Ex 4605 | 4%-6% | 4%-6% | 1%-2% | 8%-10% | 2%-4% |
| Ex 4606 | 4%-6% | 4%-6% | 1%-2% | 8%-10% | 4%-6% |
| Ex 4607 | 4%-6% | 4%-6% | 1%-2% | 8%-10% | 6%-8% |
| Ex 4608 | 4%-6% | 4%-6% | 1%-2% | 8%-10% | 8%-10% |
| Ex 4609 | 4%-6% | 4%-6% | 2%-4% | 0%-1% | 0%-1% |
| Ex 4610 | 4%-6% | 4%-6% | 2%-4% | 0%-1% | 1%-2% |
| Ex 4611 | 4%-6% | 4%-6% | 2%-4% | 0%-1% | 2%-4% |
| Ex 4612 | 4%-6% | 4%-6% | 2%-4% | 0%-1% | 4%-6% |
| Ex 4613 | 4%-6% | 4%-6% | 2%-4% | 0%-1% | 6%-8% |
| Ex 4614 | 4%-6% | 4%-6% | 2%-4% | 0%-1% | 8%-10% |
| Ex 4615 | 4%-6% | 4%-6% | 2%-4% | 1%-2% | 0%-1% |
| Ex 4616 | 4%-6% | 4%-6% | 2%-4% | 1%-2% | 1%-2% |
| Ex 4617 | 4%-6% | 4%-6% | 2%-4% | 1%-2% | 2%-4% |
| Ex 4618 | 4%-6% | 4%-6% | 2%-4% | 1%-2% | 4%-6% |
| Ex 4619 | 4%-6% | 4%-6% | 2%-4% | 1%-2% | 6%-8% |
| Ex 4620 | 4%-6% | 4%-6% | 2%-4% | 1%-2% | 8%-10% |
| Ex 4621 | 4%-6% | 4%-6% | 2%-4% | 2%-4% | 0%-1% |
| Ex 4622 | 4%-6% | 4%-6% | 2%-4% | 2%-4% | 1%-2% |
| Ex 4623 | 4%-6% | 4%-6% | 2%-4% | 2%-4% | 2%-4% |
| Ex 4624 | 4%-6% | 4%-6% | 2%-4% | 2%-4% | 4%-6% |
| Ex 4625 | 4%-6% | 4%-6% | 2%-4% | 2%-4% | 6%-8% |
| Ex 4626 | 4%-6% | 4%-6% | 2%-4% | 2%-4% | 8%-10% |
| Ex 4627 | 4%-6% | 4%-6% | 2%-4% | 4%-6% | 0%-1% |
| Ex 4628 | 4%-6% | 4%-6% | 2%-4% | 4%-6% | 1%-2% |
| Ex 4629 | 4%-6% | 4%-6% | 2%-4% | 4%-6% | 2%-4% |
| Ex 4630 | 4%-6% | 4%-6% | 2%-4% | 4%-6% | 4%-6% |
| Ex 4631 | 4%-6% | 4%-6% | 2%-4% | 4%-6% | 6%-8% |
| Ex 4632 | 4%-6% | 4%-6% | 2%-4% | 4%-6% | 8%-10% |
| Ex 4633 | 4%-6% | 4%-6% | 2%-4% | 6%-8% | 0%-1% |
| Ex 4634 | 4%-6% | 4%-6% | 2%-4% | 6%-8% | 1%-2% |
| Ex 4635 | 4%-6% | 4%-6% | 2%-4% | 6%-8% | 2%-4% |
| Ex 4636 | 4%-6% | 4%-6% | 2%-4% | 6%-8% | 4%-6% |
| Ex 4637 | 4%-6% | 4%-6% | 2%-4% | 6%-8% | 6%-8% |
| Ex 4638 | 4%-6% | 4%-6% | 2%-4% | 6%-8% | 8%-10% |
| Ex 4639 | 4%-6% | 4%-6% | 2%-4% | 8%-10% | 0%-1% |
| Ex 4640 | 4%-6% | 4%-6% | 2%-4% | 8%-10% | 1%-2% |
| Ex 4641 | 4%-6% | 4%-6% | 2%-4% | 8%-10% | 2%-4% |
| Ex 4642 | 4%-6% | 4%-6% | 2%-4% | 8%-10% | 4%-6% |
| Ex 4643 | 4%-6% | 4%-6% | 2%-4% | 8%-10% | 6%-8% |
| Ex 4644 | 4%-6% | 4%-6% | 2%-4% | 8%-10% | 8%-10% |
| Ex 4645 | 4%-6% | 4%-6% | 4%-6% | 0%-1% | 0%-1% |
| Ex 4646 | 4%-6% | 4%-6% | 4%-6% | 0%-1% | 1%-2% |
| Ex 4647 | 4%-6% | 4%-6% | 4%-6% | 0%-1% | 2%-4% |
| Ex 4648 | 4%-6% | 4%-6% | 4%-6% | 0%-1% | 4%-6% |
| Ex 4649 | 4%-6% | 4%-6% | 4%-6% | 0%-1% | 6%-8% |
| Ex 4650 | 4%-6% | 4%-6% | 4%-6% | 0%-1% | 8%-10% |
| Ex 4651 | 4%-6% | 4%-6% | 4%-6% | 1%-2% | 0%-1% |
| Ex 4652 | 4%-6% | 4%-6% | 4%-6% | 1%-2% | 1%-2% |
| Ex 4653 | 4%-6% | 4%-6% | 4%-6% | 1%-2% | 2%-4% |
| Ex 4654 | 4%-6% | 4%-6% | 4%-6% | 1%-2% | 4%-6% |
| Ex 4655 | 4%-6% | 4%-6% | 4%-6% | 1%-2% | 6%-8% |
| Ex 4656 | 4%-6% | 4%-6% | 4%-6% | 1%-2% | 8%-10% |
| Ex 4657 | 4%-6% | 4%-6% | 4%-6% | 2%-4% | 0%-1% |
| Ex 4658 | 4%-6% | 4%-6% | 4%-6% | 2%-4% | 1%-2% |
| Ex 4659 | 4%-6% | 4%-6% | 4%-6% | 2%-4% | 2%-4% |
| Ex 4660 | 4%-6% | 4%-6% | 4%-6% | 2%-4% | 4%-6% |
| Ex 4661 | 4%-6% | 4%-6% | 4%-6% | 2%-4% | 6%-8% |
| Ex 4662 | 4%-6% | 4%-6% | 4%-6% | 2%-4% | 8%-10% |
| Ex 4663 | 4%-6% | 4%-6% | 4%-6% | 4%-6% | 0%-1% |
| Ex 4664 | 4%-6% | 4%-6% | 4%-6% | 4%-6% | 1%-2% |
| Ex 4665 | 4%-6% | 4%-6% | 4%-6% | 4%-6% | 2%-4% |
| Ex 4666 | 4%-6% | 4%-6% | 4%-6% | 4%-6% | 4%-6% |
| Ex 4667 | 4%-6% | 4%-6% | 4%-6% | 4%-6% | 6%-8% |
| Ex 4668 | 4%-6% | 4%-6% | 4%-6% | 4%-6% | 8%-10% |
| Ex 4669 | 4%-6% | 4%-6% | 4%-6% | 6%-8% | 0%-1% |
| Ex 4670 | 4%-6% | 4%-6% | 4%-6% | 6%-8% | 1%-2% |
| Ex 4671 | 4%-6% | 4%-6% | 4%-6% | 6%-8% | 2%-4% |
| Ex 4672 | 4%-6% | 4%-6% | 4%-6% | 6%-8% | 4%-6% |
| Ex 4673 | 4%-6% | 4%-6% | 4%-6% | 6%-8% | 6%-8% |
| Ex 4674 | 4%-6% | 4%-6% | 4%-6% | 6%-8% | 8%-10% |
| Ex 4675 | 4%-6% | 4%-6% | 4%-6% | 8%-10% | 0%-1% |
| Ex 4676 | 4%-6% | 4%-6% | 4%-6% | 8%-10% | 1%-2% |
| Ex 4677 | 4%-6% | 4%-6% | 4%-6% | 8%-10% | 2%-4% |
| Ex 4678 | 4%-6% | 4%-6% | 4%-6% | 8%-10% | 4%-6% |
| Ex 4679 | 4%-6% | 4%-6% | 4%-6% | 8%-10% | 6%-8% |
| Ex 4680 | 4%-6% | 4%-6% | 4%-6% | 8%-10% | 8%-10% |
| Ex 4681 | 4%-6% | 4%-6% | 6%-8% | 0%-1% | 0%-1% |
| Ex 4682 | 4%-6% | 4%-6% | 6%-8% | 0%-1% | 1%-2% |
| Ex 4683 | 4%-6% | 4%-6% | 6%-8% | 0%-1% | 2%-4% |
| Ex 4684 | 4%-6% | 4%-6% | 6%-8% | 0%-1% | 4%-6% |
| Ex 4685 | 4%-6% | 4%-6% | 6%-8% | 0%-1% | 6%-8% |
| Ex 4686 | 4%-6% | 4%-6% | 6%-8% | 0%-1% | 8%-10% |
| Ex 4687 | 4%-6% | 4%-6% | 6%-8% | 1%-2% | 0%-1% |
| Ex 4688 | 4%-6% | 4%-6% | 6%-8% | 1%-2% | 1%-2% |
| Ex 4689 | 4%-6% | 4%-6% | 6%-8% | 1%-2% | 2%-4% |
| Ex 4690 | 4%-6% | 4%-6% | 6%-8% | 1%-2% | 4%-6% |
| Ex 4691 | 4%-6% | 4%-6% | 6%-8% | 1%-2% | 6%-8% |
| Ex 4692 | 4%-6% | 4%-6% | 6%-8% | 1%-2% | 8%-10% |
| Ex 4693 | 4%-6% | 4%-6% | 6%-8% | 2%-4% | 0%-1% |
| Ex 4694 | 4%-6% | 4%-6% | 6%-8% | 2%-4% | 1%-2% |
| Ex 4695 | 4%-6% | 4%-6% | 6%-8% | 2%-4% | 2%-4% |
| Ex 4696 | 4%-6% | 4%-6% | 6%-8% | 2%-4% | 4%-6% |
| Ex 4697 | 4%-6% | 4%-6% | 6%-8% | 2%-4% | 6%-8% |
| Ex 4698 | 4%-6% | 4%-6% | 6%-8% | 2%-4% | 8%-10% |
| Ex 4699 | 4%-6% | 4%-6% | 6%-8% | 4%-6% | 0%-1% |
| Ex 4700 | 4%-6% | 4%-6% | 6%-8% | 4%-6% | 1%-2% |
| Ex 4701 | 4%-6% | 4%-6% | 6%-8% | 4%-6% | 2%-4% |
| Ex 4702 | 4%-6% | 4%-6% | 6%-8% | 4%-6% | 4%-6% |
| Ex 4703 | 4%-6% | 4%-6% | 6%-8% | 4%-6% | 6%-8% |
| Ex 4704 | 4%-6% | 4%-6% | 6%-8% | 4%-6% | 8%-10% |
| Ex 4705 | 4%-6% | 4%-6% | 6%-8% | 6%-8% | 0%-1% |
| Ex 4706 | 4%-6% | 4%-6% | 6%-8% | 6%-8% | 1%-2% |
| Ex 4707 | 4%-6% | 4%-6% | 6%-8% | 6%-8% | 2%-4% |
| Ex 4708 | 4%-6% | 4%-6% | 6%-8% | 6%-8% | 4%-6% |
| Ex 4709 | 4%-6% | 4%-6% | 6%-8% | 6%-8% | 6%-8% |
| Ex 4710 | 4%-6% | 4%-6% | 6%-8% | 6%-8% | 8%-10% |
| Ex 4711 | 4%-6% | 4%-6% | 6%-8% | 8%-10% | 0%-1% |
| Ex 4712 | 4%-6% | 4%-6% | 6%-8% | 8%-10% | 1%-2% |
| Ex 4713 | 4%-6% | 4%-6% | 6%-8% | 8%-10% | 2%-4% |
| Ex 4714 | 4%-6% | 4%-6% | 6%-8% | 8%-10% | 4%-6% |
| Ex 4715 | 4%-6% | 4%-6% | 6%-8% | 8%-10% | 6%-8% |
| Ex 4716 | 4%-6% | 4%-6% | 6%-8% | 8%-10% | 8%-10% |
| Ex 4717 | 4%-6% | 4%-6% | 8%-10% | 0%-1% | 0%-1% |
| Ex 4718 | 4%-6% | 4%-6% | 8%-10% | 0%-1% | 1%-2% |
| Ex 4719 | 4%-6% | 4%-6% | 8%-10% | 0%-1% | 2%-4% |
| Ex 4720 | 4%-6% | 4%-6% | 8%-10% | 0%-1% | 4%-6% |
| Ex 4721 | 4%-6% | 4%-6% | 8%-10% | 0%-1% | 6%-8% |
| Ex 4722 | 4%-6% | 4%-6% | 8%-10% | 0%-1% | 8%-10% |
| Ex 4723 | 4%-6% | 4%-6% | 8%-10% | 1%-2% | 0%-1% |
| Ex 4724 | 4%-6% | 4%-6% | 8%-10% | 1%-2% | 1%-2% |
| Ex 4725 | 4%-6% | 4%-6% | 8%-10% | 1%-2% | 2%-4% |
| Ex 4726 | 4%-6% | 4%-6% | 8%-10% | 1%-2% | 4%-6% |
| Ex 4727 | 4%-6% | 4%-6% | 8%-10% | 1%-2% | 6%-8% |
| Ex 4728 | 4%-6% | 4%-6% | 8%-10% | 1%-2% | 8%-10% |
| Ex 4729 | 4%-6% | 4%-6% | 8%-10% | 2%-4% | 0%-1% |
| Ex 4730 | 4%-6% | 4%-6% | 8%-10% | 2%-4% | 1%-2% |
| Ex 4731 | 4%-6% | 4%-6% | 8%-10% | 2%-4% | 2%-4% |
| Ex 4732 | 4%-6% | 4%-6% | 8%-10% | 2%-4% | 4%-6% |
| Ex 4733 | 4%-6% | 4%-6% | 8%-10% | 2%-4% | 6%-8% |
| Ex 4734 | 4%-6% | 4%-6% | 8%-10% | 2%-4% | 8%-10% |
| Ex 4735 | 4%-6% | 4%-6% | 8%-10% | 4%-6% | 0%-1% |
| Ex 4736 | 4%-6% | 4%-6% | 8%-10% | 4%-6% | 1%-2% |
| Ex 4737 | 4%-6% | 4%-6% | 8%-10% | 4%-6% | 2%-4% |
| Ex 4738 | 4%-6% | 4%-6% | 8%-10% | 4%-6% | 4%-6% |
| Ex 4739 | 4%-6% | 4%-6% | 8%-10% | 4%-6% | 6%-8% |
| Ex 4740 | 4%-6% | 4%-6% | 8%-10% | 4%-6% | 8%-10% |
| Ex 4741 | 4%-6% | 4%-6% | 8%-10% | 6%-8% | 0%-1% |
| Ex 4742 | 4%-6% | 4%-6% | 8%-10% | 6%-8% | 1%-2% |
| Ex 4743 | 4%-6% | 4%-6% | 8%-10% | 6%-8% | 2%-4% |
| Ex 4744 | 4%-6% | 4%-6% | 8%-10% | 6%-8% | 4%-6% |
| Ex 4745 | 4%-6% | 4%-6% | 8%-10% | 6%-8% | 6%-8% |
| Ex 4746 | 4%-6% | 4%-6% | 8%-10% | 6%-8% | 8%-10% |
| Ex 4747 | 4%-6% | 4%-6% | 8%-10% | 8%-10% | 0%-1% |
| Ex 4748 | 4%-6% | 4%-6% | 8%-10% | 8%-10% | 1%-2% |
| Ex 4749 | 4%-6% | 4%-6% | 8%-10% | 8%-10% | 2%-4% |
| Ex 4750 | 4%-6% | 4%-6% | 8%-10% | 8%-10% | 4%-6% |
| Ex 4751 | 4%-6% | 4%-6% | 8%-10% | 8%-10% | 6%-8% |
| Ex 4752 | 4%-6% | 4%-6% | 8%-10% | 8%-10% | 8%-10% |
| Ex 4753 | 4%-6% | 6%-8% | 0%-1% | 0%-1% | 0%-1% |
| Ex 4754 | 4%-6% | 6%-8% | 0%-1% | 0%-1% | 1%-2% |
| Ex 4755 | 4%-6% | 6%-8% | 0%-1% | 0%-1% | 2%-4% |
| Ex 4756 | 4%-6% | 6%-8% | 0%-1% | 0%-1% | 4%-6% |
| Ex 4757 | 4%-6% | 6%-8% | 0%-1% | 0%-1% | 6%-8% |
| Ex 4758 | 4%-6% | 6%-8% | 0%-1% | 0%-1% | 8%-10% |
| Ex 4759 | 4%-6% | 6%-8% | 0%-1% | 1%-2% | 0%-1% |
| Ex 4760 | 4%-6% | 6%-8% | 0%-1% | 1%-2% | 1%-2% |
| Ex 4761 | 4%-6% | 6%-8% | 0%-1% | 1%-2% | 2%-4% |
| Ex 4762 | 4%-6% | 6%-8% | 0%-1% | 1%-2% | 4%-6% |
| Ex 4763 | 4%-6% | 6%-8% | 0%-1% | 1%-2% | 6%-8% |
| Ex 4764 | 4%-6% | 6%-8% | 0%-1% | 1%-2% | 8%-10% |
| Ex 4765 | 4%-6% | 6%-8% | 0%-1% | 2%-4% | 0%-1% |
| Ex 4766 | 4%-6% | 6%-8% | 0%-1% | 2%-4% | 1%-2% |
| Ex 4767 | 4%-6% | 6%-8% | 0%-1% | 2%-4% | 2%-4% |
| Ex 4768 | 4%-6% | 6%-8% | 0%-1% | 2%-4% | 4%-6% |
| Ex 4769 | 4%-6% | 6%-8% | 0%-1% | 2%-4% | 6%-8% |
| Ex 4770 | 4%-6% | 6%-8% | 0%-1% | 2%-4% | 8%-10% |
| Ex 4771 | 4%-6% | 6%-8% | 0%-1% | 4%-6% | 0%-1% |
| Ex 4772 | 4%-6% | 6%-8% | 0%-1% | 4%-6% | 1%-2% |
| Ex 4773 | 4%-6% | 6%-8% | 0%-1% | 4%-6% | 2%-4% |
| Ex 4774 | 4%-6% | 6%-8% | 0%-1% | 4%-6% | 4%-6% |
| Ex 4775 | 4%-6% | 6%-8% | 0%-1% | 4%-6% | 6%-8% |
| Ex 4776 | 4%-6% | 6%-8% | 0%-1% | 4%-6% | 8%-10% |
| Ex 4777 | 4%-6% | 6%-8% | 0%-1% | 6%-8% | 0%-1% |
| Ex 4778 | 4%-6% | 6%-8% | 0%-1% | 6%-8% | 1%-2% |
| Ex 4779 | 4%-6% | 6%-8% | 0%-1% | 6%-8% | 2%-4% |
| Ex 4780 | 4%-6% | 6%-8% | 0%-1% | 6%-8% | 4%-6% |
| Ex 4781 | 4%-6% | 6%-8% | 0%-1% | 6%-8% | 6%-8% |
| Ex 4782 | 4%-6% | 6%-8% | 0%-1% | 6%-8% | 8%-10% |
| Ex 4783 | 4%-6% | 6%-8% | 0%-1% | 8%-10% | 0%-1% |
| Ex 4784 | 4%-6% | 6%-8% | 0%-1% | 8%-10% | 1%-2% |
| Ex 4785 | 4%-6% | 6%-8% | 0%-1% | 8%-10% | 2%-4% |
| Ex 4786 | 4%-6% | 6%-8% | 0%-1% | 8%-10% | 4%-6% |
| Ex 4787 | 4%-6% | 6%-8% | 0%-1% | 8%-10% | 6%-8% |
| Ex 4788 | 4%-6% | 6%-8% | 0%-1% | 8%-10% | 8%-10% |
| Ex 4789 | 4%-6% | 6%-8% | 1%-2% | 0%-1% | 0%-1% |
| Ex 4790 | 4%-6% | 6%-8% | 1%-2% | 0%-1% | 1%-2% |
| Ex 4791 | 4%-6% | 6%-8% | 1%-2% | 0%-1% | 2%-4% |
| Ex 4792 | 4%-6% | 6%-8% | 1%-2% | 0%-1% | 4%-6% |
| Ex 4793 | 4%-6% | 6%-8% | 1%-2% | 0%-1% | 6%-8% |
| Ex 4794 | 4%-6% | 6%-8% | 1%-2% | 0%-1% | 8%-10% |
| Ex 4795 | 4%-6% | 6%-8% | 1%-2% | 1%-2% | 0%-1% |
| Ex 4796 | 4%-6% | 6%-8% | 1%-2% | 1%-2% | 1%-2% |
| Ex 4797 | 4%-6% | 6%-8% | 1%-2% | 1%-2% | 2%-4% |
| Ex 4798 | 4%-6% | 6%-8% | 1%-2% | 1%-2% | 4%-6% |
| Ex 4799 | 4%-6% | 6%-8% | 1%-2% | 1%-2% | 6%-8% |
| Ex 4800 | 4%-6% | 6%-8% | 1%-2% | 1%-2% | 8%-10% |
| Ex 4801 | 4%-6% | 6%-8% | 1%-2% | 2%-4% | 0%-1% |
| Ex 4802 | 4%-6% | 6%-8% | 1%-2% | 2%-4% | 1%-2% |
| Ex 4803 | 4%-6% | 6%-8% | 1%-2% | 2%-4% | 2%-4% |
| Ex 4804 | 4%-6% | 6%-8% | 1%-2% | 2%-4% | 4%-6% |
| Ex 4805 | 4%-6% | 6%-8% | 1%-2% | 2%-4% | 6%-8% |
| Ex 4806 | 4%-6% | 6%-8% | 1%-2% | 2%-4% | 8%-10% |
| Ex 4807 | 4%-6% | 6%-8% | 1%-2% | 4%-6% | 0%-1% |
| Ex 4808 | 4%-6% | 6%-8% | 1%-2% | 4%-6% | 1%-2% |
| Ex 4809 | 4%-6% | 6%-8% | 1%-2% | 4%-6% | 2%-4% |
| Ex 4810 | 4%-6% | 6%-8% | 1%-2% | 4%-6% | 4%-6% |
| Ex 4811 | 4%-6% | 6%-8% | 1%-2% | 4%-6% | 6%-8% |
| Ex 4812 | 4%-6% | 6%-8% | 1%-2% | 4%-6% | 8%-10% |
| Ex 4813 | 4%-6% | 6%-8% | 1%-2% | 6%-8% | 0%-1% |
| Ex 4814 | 4%-6% | 6%-8% | 1%-2% | 6%-8% | 1%-2% |
| Ex 4815 | 4%-6% | 6%-8% | 1%-2% | 6%-8% | 2%-4% |
| Ex 4816 | 4%-6% | 6%-8% | 1%-2% | 6%-8% | 4%-6% |
| Ex 4817 | 4%-6% | 6%-8% | 1%-2% | 6%-8% | 6%-8% |
| Ex 4818 | 4%-6% | 6%-8% | 1%-2% | 6%-8% | 8%-10% |
| Ex 4819 | 4%-6% | 6%-8% | 1%-2% | 8%-10% | 0%-1% |
| Ex 4820 | 4%-6% | 6%-8% | 1%-2% | 8%-10% | 1%-2% |
| Ex 4821 | 4%-6% | 6%-8% | 1%-2% | 8%-10% | 2%-4% |
| Ex 4822 | 4%-6% | 6%-8% | 1%-2% | 8%-10% | 4%-6% |
| Ex 4823 | 4%-6% | 6%-8% | 1%-2% | 8%-10% | 6%-8% |
| Ex 4824 | 4%-6% | 6%-8% | 1%-2% | 8%-10% | 8%-10% |
| Ex 4825 | 4%-6% | 6%-8% | 2%-4% | 0%-1% | 0%-1% |
| Ex 4826 | 4%-6% | 6%-8% | 2%-4% | 0%-1% | 1%-2% |
| Ex 4827 | 4%-6% | 6%-8% | 2%-4% | 0%-1% | 2%-4% |
| Ex 4828 | 4%-6% | 6%-8% | 2%-4% | 0%-1% | 4%-6% |
| Ex 4829 | 4%-6% | 6%-8% | 2%-4% | 0%-1% | 6%-8% |
| Ex 4830 | 4%-6% | 6%-8% | 2%-4% | 0%-1% | 8%-10% |
| Ex 4831 | 4%-6% | 6%-8% | 2%-4% | 1%-2% | 0%-1% |
| Ex 4832 | 4%-6% | 6%-8% | 2%-4% | 1%-2% | 1%-2% |
| Ex 4833 | 4%-6% | 6%-8% | 2%-4% | 1%-2% | 2%-4% |
| Ex 4834 | 4%-6% | 6%-8% | 2%-4% | 1%-2% | 4%-6% |
| Ex 4835 | 4%-6% | 6%-8% | 2%-4% | 1%-2% | 6%-8% |
| Ex 4836 | 4%-6% | 6%-8% | 2%-4% | 1%-2% | 8%-10% |
| Ex 4837 | 4%-6% | 6%-8% | 2%-4% | 2%-4% | 0%-1% |
| Ex 4838 | 4%-6% | 6%-8% | 2%-4% | 2%-4% | 1%-2% |
| Ex 4839 | 4%-6% | 6%-8% | 2%-4% | 2%-4% | 2%-4% |
| Ex 4840 | 4%-6% | 6%-8% | 2%-4% | 2%-4% | 4%-6% |
| Ex 4841 | 4%-6% | 6%-8% | 2%-4% | 2%-4% | 6%-8% |
| Ex 4842 | 4%-6% | 6%-8% | 2%-4% | 2%-4% | 8%-10% |
| Ex 4843 | 4%-6% | 6%-8% | 2%-4% | 4%-6% | 0%-1% |
| Ex 4844 | 4%-6% | 6%-8% | 2%-4% | 4%-6% | 1%-2% |
| Ex 4845 | 4%-6% | 6%-8% | 2%-4% | 4%-6% | 2%-4% |
| Ex 4846 | 4%-6% | 6%-8% | 2%-4% | 4%-6% | 4%-6% |
| Ex 4847 | 4%-6% | 6%-8% | 2%-4% | 4%-6% | 6%-8% |
| Ex 4848 | 4%-6% | 6%-8% | 2%-4% | 4%-6% | 8%-10% |
| Ex 4849 | 4%-6% | 6%-8% | 2%-4% | 6%-8% | 0%-1% |
| Ex 4850 | 4%-6% | 6%-8% | 2%-4% | 6%-8% | 1%-2% |
| Ex 4851 | 4%-6% | 6%-8% | 2%-4% | 6%-8% | 2%-4% |
| Ex 4852 | 4%-6% | 6%-8% | 2%-4% | 6%-8% | 4%-6% |
| Ex 4853 | 4%-6% | 6%-8% | 2%-4% | 6%-8% | 6%-8% |
| Ex 4854 | 4%-6% | 6%-8% | 2%-4% | 6%-8% | 8%-10% |
| Ex 4855 | 4%-6% | 6%-8% | 2%-4% | 8%-10% | 0%-1% |
| Ex 4856 | 4%-6% | 6%-8% | 2%-4% | 8%-10% | 1%-2% |
| Ex 4857 | 4%-6% | 6%-8% | 2%-4% | 8%-10% | 2%-4% |
| Ex 4858 | 4%-6% | 6%-8% | 2%-4% | 8%-10% | 4%-6% |
| Ex 4859 | 4%-6% | 6%-8% | 2%-4% | 8%-10% | 6%-8% |
| Ex 4860 | 4%-6% | 6%-8% | 2%-4% | 8%-10% | 8%-10% |
| Ex 4861 | 4%-6% | 6%-8% | 4%-6% | 0%-1% | 0%-1% |
| Ex 4862 | 4%-6% | 6%-8% | 4%-6% | 0%-1% | 1%-2% |
| Ex 4863 | 4%-6% | 6%-8% | 4%-6% | 0%-1% | 2%-4% |
| Ex 4864 | 4%-6% | 6%-8% | 4%-6% | 0%-1% | 4%-6% |
| Ex 4865 | 4%-6% | 6%-8% | 4%-6% | 0%-1% | 6%-8% |
| Ex 4866 | 4%-6% | 6%-8% | 4%-6% | 0%-1% | 8%-10% |
| Ex 4867 | 4%-6% | 6%-8% | 4%-6% | 1%-2% | 0%-1% |
| Ex 4868 | 4%-6% | 6%-8% | 4%-6% | 1%-2% | 1%-2% |
| Ex 4869 | 4%-6% | 6%-8% | 4%-6% | 1%-2% | 2%-4% |
| Ex 4870 | 4%-6% | 6%-8% | 4%-6% | 1%-2% | 4%-6% |
| Ex 4871 | 4%-6% | 6%-8% | 4%-6% | 1%-2% | 6%-8% |
| Ex 4872 | 4%-6% | 6%-8% | 4%-6% | 1%-2% | 8%-10% |
| Ex 4873 | 4%-6% | 6%-8% | 4%-6% | 2%-4% | 0%-1% |
| Ex 4874 | 4%-6% | 6%-8% | 4%-6% | 2%-4% | 1%-2% |
| Ex 4875 | 4%-6% | 6%-8% | 4%-6% | 2%-4% | 2%-4% |
| Ex 4876 | 4%-6% | 6%-8% | 4%-6% | 2%-4% | 4%-6% |
| Ex 4877 | 4%-6% | 6%-8% | 4%-6% | 2%-4% | 6%-8% |
| Ex 4878 | 4%-6% | 6%-8% | 4%-6% | 2%-4% | 8%-10% |
| Ex 4879 | 4%-6% | 6%-8% | 4%-6% | 4%-6% | 0%-1% |
| Ex 4880 | 4%-6% | 6%-8% | 4%-6% | 4%-6% | 1%-2% |
| Ex 4881 | 4%-6% | 6%-8% | 4%-6% | 4%-6% | 2%-4% |
| Ex 4882 | 4%-6% | 6%-8% | 4%-6% | 4%-6% | 4%-6% |
| Ex 4883 | 4%-6% | 6%-8% | 4%-6% | 4%-6% | 6%-8% |
| Ex 4884 | 4%-6% | 6%-8% | 4%-6% | 4%-6% | 8%-10% |
| Ex 4885 | 4%-6% | 6%-8% | 4%-6% | 6%-8% | 0%-1% |
| Ex 4886 | 4%-6% | 6%-8% | 4%-6% | 6%-8% | 1%-2% |
| Ex 4887 | 4%-6% | 6%-8% | 4%-6% | 6%-8% | 2%-4% |
| Ex 4888 | 4%-6% | 6%-8% | 4%-6% | 6%-8% | 4%-6% |
| Ex 4889 | 4%-6% | 6%-8% | 4%-6% | 6%-8% | 6%-8% |
| Ex 4890 | 4%-6% | 6%-8% | 4%-6% | 6%-8% | 8%-10% |
| Ex 4891 | 4%-6% | 6%-8% | 4%-6% | 8%-10% | 0%-1% |
| Ex 4892 | 4%-6% | 6%-8% | 4%-6% | 8%-10% | 1%-2% |
| Ex 4893 | 4%-6% | 6%-8% | 4%-6% | 8%-10% | 2%-4% |
| Ex 4894 | 4%-6% | 6%-8% | 4%-6% | 8%-10% | 4%-6% |
| Ex 4895 | 4%-6% | 6%-8% | 4%-6% | 8%-10% | 6%-8% |
| Ex 4896 | 4%-6% | 6%-8% | 4%-6% | 8%-10% | 8%-10% |
| Ex 4897 | 4%-6% | 6%-8% | 6%-8% | 0%-1% | 0%-1% |
| Ex 4898 | 4%-6% | 6%-8% | 6%-8% | 0%-1% | 1%-2% |
| Ex 4899 | 4%-6% | 6%-8% | 6%-8% | 0%-1% | 2%-4% |
| Ex 4900 | 4%-6% | 6%-8% | 6%-8% | 0%-1% | 4%-6% |
| Ex 4901 | 4%-6% | 6%-8% | 6%-8% | 0%-1% | 6%-8% |
| Ex 4902 | 4%-6% | 6%-8% | 6%-8% | 0%-1% | 8%-10% |
| Ex 4903 | 4%-6% | 6%-8% | 6%-8% | 1%-2% | 0%-1% |
| Ex 4904 | 4%-6% | 6%-8% | 6%-8% | 1%-2% | 1%-2% |
| Ex 4905 | 4%-6% | 6%-8% | 6%-8% | 1%-2% | 2%-4% |
| Ex 4906 | 4%-6% | 6%-8% | 6%-8% | 1%-2% | 4%-6% |
| Ex 4907 | 4%-6% | 6%-8% | 6%-8% | 1%-2% | 6%-8% |
| Ex 4908 | 4%-6% | 6%-8% | 6%-8% | 1%-2% | 8%-10% |
| Ex 4909 | 4%-6% | 6%-8% | 6%-8% | 2%-4% | 0%-1% |
| Ex 4910 | 4%-6% | 6%-8% | 6%-8% | 2%-4% | 1%-2% |
| Ex 4911 | 4%-6% | 6%-8% | 6%-8% | 2%-4% | 2%-4% |
| Ex 4912 | 4%-6% | 6%-8% | 6%-8% | 2%-4% | 4%-6% |
| Ex 4913 | 4%-6% | 6%-8% | 6%-8% | 2%-4% | 6%-8% |
| Ex 4914 | 4%-6% | 6%-8% | 6%-8% | 2%-4% | 8%-10% |
| Ex 4915 | 4%-6% | 6%-8% | 6%-8% | 4%-6% | 0%-1% |
| Ex 4916 | 4%-6% | 6%-8% | 6%-8% | 4%-6% | 1%-2% |
| Ex 4917 | 4%-6% | 6%-8% | 6%-8% | 4%-6% | 2%-4% |
| Ex 4918 | 4%-6% | 6%-8% | 6%-8% | 4%-6% | 4%-6% |
| Ex 4919 | 4%-6% | 6%-8% | 6%-8% | 4%-6% | 6%-8% |
| Ex 4920 | 4%-6% | 6%-8% | 6%-8% | 4%-6% | 8%-10% |
| Ex 4921 | 4%-6% | 6%-8% | 6%-8% | 6%-8% | 0%-1% |
| Ex 4922 | 4%-6% | 6%-8% | 6%-8% | 6%-8% | 1%-2% |
| Ex 4923 | 4%-6% | 6%-8% | 6%-8% | 6%-8% | 2%-4% |
| Ex 4924 | 4%-6% | 6%-8% | 6%-8% | 6%-8% | 4%-6% |
| Ex 4925 | 4%-6% | 6%-8% | 6%-8% | 6%-8% | 6%-8% |
| Ex 4926 | 4%-6% | 6%-8% | 6%-8% | 6%-8% | 8%-10% |
| Ex 4927 | 4%-6% | 6%-8% | 6%-8% | 8%-10% | 0%-1% |
| Ex 4928 | 4%-6% | 6%-8% | 6%-8% | 8%-10% | 1%-2% |
| Ex 4929 | 4%-6% | 6%-8% | 6%-8% | 8%-10% | 2%-4% |
| Ex 4930 | 4%-6% | 6%-8% | 6%-8% | 8%-10% | 4%-6% |
| Ex 4931 | 4%-6% | 6%-8% | 6%-8% | 8%-10% | 6%-8% |
| Ex 4932 | 4%-6% | 6%-8% | 6%-8% | 8%-10% | 8%-10% |
| Ex 4933 | 4%-6% | 6%-8% | 8%-10% | 0%-1% | 0%-1% |
| Ex 4934 | 4%-6% | 6%-8% | 8%-10% | 0%-1% | 1%-2% |
| Ex 4935 | 4%-6% | 6%-8% | 8%-10% | 0%-1% | 2%-4% |
| Ex 4936 | 4%-6% | 6%-8% | 8%-10% | 0%-1% | 4%-6% |
| Ex 4937 | 4%-6% | 6%-8% | 8%-10% | 0%-1% | 6%-8% |
| Ex 4938 | 4%-6% | 6%-8% | 8%-10% | 0%-1% | 8%-10% |
| Ex 4939 | 4%-6% | 6%-8% | 8%-10% | 1%-2% | 0%-1% |
| Ex 4940 | 4%-6% | 6%-8% | 8%-10% | 1%-2% | 1%-2% |
| Ex 4941 | 4%-6% | 6%-8% | 8%-10% | 1%-2% | 2%-4% |
| Ex 4942 | 4%-6% | 6%-8% | 8%-10% | 1%-2% | 4%-6% |
| Ex 4943 | 4%-6% | 6%-8% | 8%-10% | 1%-2% | 6%-8% |
| Ex 4944 | 4%-6% | 6%-8% | 8%-10% | 1%-2% | 8%-10% |
| Ex 4945 | 4%-6% | 6%-8% | 8%-10% | 2%-4% | 0%-1% |
| Ex 4946 | 4%-6% | 6%-8% | 8%-10% | 2%-4% | 1%-2% |
| Ex 4947 | 4%-6% | 6%-8% | 8%-10% | 2%-4% | 2%-4% |
| Ex 4948 | 4%-6% | 6%-8% | 8%-10% | 2%-4% | 4%-6% |
| Ex 4949 | 4%-6% | 6%-8% | 8%-10% | 2%-4% | 6%-8% |
| Ex 4950 | 4%-6% | 6%-8% | 8%-10% | 2%-4% | 8%-10% |
| Ex 4951 | 4%-6% | 6%-8% | 8%-10% | 4%-6% | 0%-1% |
| Ex 4952 | 4%-6% | 6%-8% | 8%-10% | 4%-6% | 1%-2% |
| Ex 4953 | 4%-6% | 6%-8% | 8%-10% | 4%-6% | 2%-4% |
| Ex 4954 | 4%-6% | 6%-8% | 8%-10% | 4%-6% | 4%-6% |
| Ex 4955 | 4%-6% | 6%-8% | 8%-10% | 4%-6% | 6%-8% |
| Ex 4956 | 4%-6% | 6%-8% | 8%-10% | 4%-6% | 8%-10% |
| Ex 4957 | 4%-6% | 6%-8% | 8%-10% | 6%-8% | 0%-1% |
| Ex 4958 | 4%-6% | 6%-8% | 8%-10% | 6%-8% | 1%-2% |
| Ex 4959 | 4%-6% | 6%-8% | 8%-10% | 6%-8% | 2%-4% |
| Ex 4960 | 4%-6% | 6%-8% | 8%-10% | 6%-8% | 4%-6% |
| Ex 4961 | 4%-6% | 6%-8% | 8%-10% | 6%-8% | 6%-8% |
| Ex 4962 | 4%-6% | 6%-8% | 8%-10% | 6%-8% | 8%-10% |
| Ex 4963 | 4%-6% | 6%-8% | 8%-10% | 8%-10% | 0%-1% |
| Ex 4964 | 4%-6% | 6%-8% | 8%-10% | 8%-10% | 1%-2% |
| Ex 4965 | 4%-6% | 6%-8% | 8%-10% | 8%-10% | 2%-4% |
| Ex 4966 | 4%-6% | 6%-8% | 8%-10% | 8%-10% | 4%-6% |
| Ex 4967 | 4%-6% | 6%-8% | 8%-10% | 8%-10% | 6%-8% |
| Ex 4968 | 4%-6% | 6%-8% | 8%-10% | 8%-10% | 8%-10% |
| Ex 4969 | 4%-6% | 8%-10% | 0%-1% | 0%-1% | 0%-1% |
| Ex 4970 | 4%-6% | 8%-10% | 0%-1% | 0%-1% | 1%-2% |
| Ex 4971 | 4%-6% | 8%-10% | 0%-1% | 0%-1% | 2%-4% |
| Ex 4972 | 4%-6% | 8%-10% | 0%-1% | 0%-1% | 4%-6% |
| Ex 4973 | 4%-6% | 8%-10% | 0%-1% | 0%-1% | 6%-8% |
| Ex 4974 | 4%-6% | 8%-10% | 0%-1% | 0%-1% | 8%-10% |
| Ex 4975 | 4%-6% | 8%-10% | 0%-1% | 1%-2% | 0%-1% |
| Ex 4976 | 4%-6% | 8%-10% | 0%-1% | 1%-2% | 1%-2% |
| Ex 4977 | 4%-6% | 8%-10% | 0%-1% | 1%-2% | 2%-4% |
| Ex 4978 | 4%-6% | 8%-10% | 0%-1% | 1%-2% | 4%-6% |
| Ex 4979 | 4%-6% | 8%-10% | 0%-1% | 1%-2% | 6%-8% |
| Ex 4980 | 4%-6% | 8%-10% | 0%-1% | 1%-2% | 8%-10% |
| Ex 4981 | 4%-6% | 8%-10% | 0%-1% | 2%-4% | 0%-1% |
| Ex 4982 | 4%-6% | 8%-10% | 0%-1% | 2%-4% | 1%-2% |
| Ex 4983 | 4%-6% | 8%-10% | 0%-1% | 2%-4% | 2%-4% |
| Ex 4984 | 4%-6% | 8%-10% | 0%-1% | 2%-4% | 4%-6% |
| Ex 4985 | 4%-6% | 8%-10% | 0%-1% | 2%-4% | 6%-8% |
| Ex 4986 | 4%-6% | 8%-10% | 0%-1% | 2%-4% | 8%-10% |
| Ex 4987 | 4%-6% | 8%-10% | 0%-1% | 4%-6% | 0%-1% |
| Ex 4988 | 4%-6% | 8%-10% | 0%-1% | 4%-6% | 1%-2% |
| Ex 4989 | 4%-6% | 8%-10% | 0%-1% | 4%-6% | 2%-4% |
| Ex 4990 | 4%-6% | 8%-10% | 0%-1% | 4%-6% | 4%-6% |
| Ex 4991 | 4%-6% | 8%-10% | 0%-1% | 4%-6% | 6%-8% |
| Ex 4992 | 4%-6% | 8%-10% | 0%-1% | 4%-6% | 8%-10% |
| Ex 4993 | 4%-6% | 8%-10% | 0%-1% | 6%-8% | 0%-1% |
| Ex 4994 | 4%-6% | 8%-10% | 0%-1% | 6%-8% | 1%-2% |
| Ex 4995 | 4%-6% | 8%-10% | 0%-1% | 6%-8% | 2%-4% |
| Ex 4996 | 4%-6% | 8%-10% | 0%-1% | 6%-8% | 4%-6% |
| Ex 4997 | 4%-6% | 8%-10% | 0%-1% | 6%-8% | 6%-8% |
| Ex 4998 | 4%-6% | 8%-10% | 0%-1% | 6%-8% | 8%-10% |
| Ex 4999 | 4%-6% | 8%-10% | 0%-1% | 8%-10% | 0%-1% |
| Ex 5000 | 4%-6% | 8%-10% | 0%-1% | 8%-10% | 1%-2% |
| Ex 5001 | 4%-6% | 8%-10% | 0%-1% | 8%-10% | 2%-4% |
| Ex 5002 | 4%-6% | 8%-10% | 0%-1% | 8%-10% | 4%-6% |
| Ex 5003 | 4%-6% | 8%-10% | 0%-1% | 8%-10% | 6%-8% |
| Ex 5004 | 4%-6% | 8%-10% | 0%-1% | 8%-10% | 8%-10% |
| Ex 5005 | 4%-6% | 8%-10% | 1%-2% | 0%-1% | 0%-1% |
| Ex 5006 | 4%-6% | 8%-10% | 1%-2% | 0%-1% | 1%-2% |
| Ex 5007 | 4%-6% | 8%-10% | 1%-2% | 0%-1% | 2%-4% |
| Ex 5008 | 4%-6% | 8%-10% | 1%-2% | 0%-1% | 4%-6% |
| Ex 5009 | 4%-6% | 8%-10% | 1%-2% | 0%-1% | 6%-8% |
| Ex 5010 | 4%-6% | 8%-10% | 1%-2% | 0%-1% | 8%-10% |
| Ex 5011 | 4%-6% | 8%-10% | 1%-2% | 1%-2% | 0%-1% |
| Ex 5012 | 4%-6% | 8%-10% | 1%-2% | 1%-2% | 1%-2% |
| Ex 5013 | 4%-6% | 8%-10% | 1%-2% | 1%-2% | 2%-4% |
| Ex 5014 | 4%-6% | 8%-10% | 1%-2% | 1%-2% | 4%-6% |
| Ex 5015 | 4%-6% | 8%-10% | 1%-2% | 1%-2% | 6%-8% |
| Ex 5016 | 4%-6% | 8%-10% | 1%-2% | 1%-2% | 8%-10% |
| Ex 5017 | 4%-6% | 8%-10% | 1%-2% | 2%-4% | 0%-1% |
| Ex 5018 | 4%-6% | 8%-10% | 1%-2% | 2%-4% | 1%-2% |
| Ex 5019 | 4%-6% | 8%-10% | 1%-2% | 2%-4% | 2%-4% |
| Ex 5020 | 4%-6% | 8%-10% | 1%-2% | 2%-4% | 4%-6% |
| Ex 5021 | 4%-6% | 8%-10% | 1%-2% | 2%-4% | 6%-8% |
| Ex 5022 | 4%-6% | 8%-10% | 1%-2% | 2%-4% | 8%-10% |
| Ex 5023 | 4%-6% | 8%-10% | 1%-2% | 4%-6% | 0%-1% |
| Ex 5024 | 4%-6% | 8%-10% | 1%-2% | 4%-6% | 1%-2% |
| Ex 5025 | 4%-6% | 8%-10% | 1%-2% | 4%-6% | 2%-4% |
| Ex 5026 | 4%-6% | 8%-10% | 1%-2% | 4%-6% | 4%-6% |
| Ex 5027 | 4%-6% | 8%-10% | 1%-2% | 4%-6% | 6%-8% |
| Ex 5028 | 4%-6% | 8%-10% | 1%-2% | 4%-6% | 8%-10% |
| Ex 5029 | 4%-6% | 8%-10% | 1%-2% | 6%-8% | 0%-1% |
| Ex 5030 | 4%-6% | 8%-10% | 1%-2% | 6%-8% | 1%-2% |
| Ex 5031 | 4%-6% | 8%-10% | 1%-2% | 6%-8% | 2%-4% |
| Ex 5032 | 4%-6% | 8%-10% | 1%-2% | 6%-8% | 4%-6% |
| Ex 5033 | 4%-6% | 8%-10% | 1%-2% | 6%-8% | 6%-8% |
| Ex 5034 | 4%-6% | 8%-10% | 1%-2% | 6%-8% | 8%-10% |
| Ex 5035 | 4%-6% | 8%-10% | 1%-2% | 8%-10% | 0%-1% |
| Ex 5036 | 4%-6% | 8%-10% | 1%-2% | 8%-10% | 1%-2% |
| Ex 5037 | 4%-6% | 8%-10% | 1%-2% | 8%-10% | 2%-4% |
| Ex 5038 | 4%-6% | 8%-10% | 1%-2% | 8%-10% | 4%-6% |
| Ex 5039 | 4%-6% | 8%-10% | 1%-2% | 8%-10% | 6%-8% |
| Ex 5040 | 4%-6% | 8%-10% | 1%-2% | 8%-10% | 8%-10% |
| Ex 5041 | 4%-6% | 8%-10% | 2%-4% | 0%-1% | 0%-1% |
| Ex 5042 | 4%-6% | 8%-10% | 2%-4% | 0%-1% | 1%-2% |
| Ex 5043 | 4%-6% | 8%-10% | 2%-4% | 0%-1% | 2%-4% |
| Ex 5044 | 4%-6% | 8%-10% | 2%-4% | 0%-1% | 4%-6% |
| Ex 5045 | 4%-6% | 8%-10% | 2%-4% | 0%-1% | 6%-8% |
| Ex 5046 | 4%-6% | 8%-10% | 2%-4% | 0%-1% | 8%-10% |
| Ex 5047 | 4%-6% | 8%-10% | 2%-4% | 1%-2% | 0%-1% |
| Ex 5048 | 4%-6% | 8%-10% | 2%-4% | 1%-2% | 1%-2% |
| Ex 5049 | 4%-6% | 8%-10% | 2%-4% | 1%-2% | 2%-4% |
| Ex 5050 | 4%-6% | 8%-10% | 2%-4% | 1%-2% | 4%-6% |
| Ex 5051 | 4%-6% | 8%-10% | 2%-4% | 1%-2% | 6%-8% |
| Ex 5052 | 4%-6% | 8%-10% | 2%-4% | 1%-2% | 8%-10% |
| Ex 5053 | 4%-6% | 8%-10% | 2%-4% | 2%-4% | 0%-1% |
| Ex 5054 | 4%-6% | 8%-10% | 2%-4% | 2%-4% | 1%-2% |
| Ex 5055 | 4%-6% | 8%-10% | 2%-4% | 2%-4% | 2%-4% |
| Ex 5056 | 4%-6% | 8%-10% | 2%-4% | 2%-4% | 4%-6% |
| Ex 5057 | 4%-6% | 8%-10% | 2%-4% | 2%-4% | 6%-8% |
| Ex 5058 | 4%-6% | 8%-10% | 2%-4% | 2%-4% | 8%-10% |
| Ex 5059 | 4%-6% | 8%-10% | 2%-4% | 4%-6% | 0%-1% |
| Ex 5060 | 4%-6% | 8%-10% | 2%-4% | 4%-6% | 1%-2% |
| Ex 5061 | 4%-6% | 8%-10% | 2%-4% | 4%-6% | 2%-4% |
| Ex 5062 | 4%-6% | 8%-10% | 2%-4% | 4%-6% | 4%-6% |
| Ex 5063 | 4%-6% | 8%-10% | 2%-4% | 4%-6% | 6%-8% |
| Ex 5064 | 4%-6% | 8%-10% | 2%-4% | 4%-6% | 8%-10% |
| Ex 5065 | 4%-6% | 8%-10% | 2%-4% | 6%-8% | 0%-1% |
| Ex 5066 | 4%-6% | 8%-10% | 2%-4% | 6%-8% | 1%-2% |
| Ex 5067 | 4%-6% | 8%-10% | 2%-4% | 6%-8% | 2%-4% |
| Ex 5068 | 4%-6% | 8%-10% | 2%-4% | 6%-8% | 4%-6% |
| Ex 5069 | 4%-6% | 8%-10% | 2%-4% | 6%-8% | 6%-8% |
| Ex 5070 | 4%-6% | 8%-10% | 2%-4% | 6%-8% | 8%-10% |
| Ex 5071 | 4%-6% | 8%-10% | 2%-4% | 8%-10% | 0%-1% |
| Ex 5072 | 4%-6% | 8%-10% | 2%-4% | 8%-10% | 1%-2% |
| Ex 5073 | 4%-6% | 8%-10% | 2%-4% | 8%-10% | 2%-4% |
| Ex 5074 | 4%-6% | 8%-10% | 2%-4% | 8%-10% | 4%-6% |
| Ex 5075 | 4%-6% | 8%-10% | 2%-4% | 8%-10% | 6%-8% |
| Ex 5076 | 4%-6% | 8%-10% | 2%-4% | 8%-10% | 8%-10% |
| Ex 5077 | 4%-6% | 8%-10% | 4%-6% | 0%-1% | 0%-1% |
| Ex 5078 | 4%-6% | 8%-10% | 4%-6% | 0%-1% | 1%-2% |
| Ex 5079 | 4%-6% | 8%-10% | 4%-6% | 0%-1% | 2%-4% |
| Ex 5080 | 4%-6% | 8%-10% | 4%-6% | 0%-1% | 4%-6% |
| Ex 5081 | 4%-6% | 8%-10% | 4%-6% | 0%-1% | 6%-8% |
| Ex 5082 | 4%-6% | 8%-10% | 4%-6% | 0%-1% | 8%-10% |
| Ex 5083 | 4%-6% | 8%-10% | 4%-6% | 1%-2% | 0%-1% |
| Ex 5084 | 4%-6% | 8%-10% | 4%-6% | 1%-2% | 1%-2% |
| Ex 5085 | 4%-6% | 8%-10% | 4%-6% | 1%-2% | 2%-4% |
| Ex 5086 | 4%-6% | 8%-10% | 4%-6% | 1%-2% | 4%-6% |
| Ex 5087 | 4%-6% | 8%-10% | 4%-6% | 1%-2% | 6%-8% |
| Ex 5088 | 4%-6% | 8%-10% | 4%-6% | 1%-2% | 8%-10% |
| Ex 5089 | 4%-6% | 8%-10% | 4%-6% | 2%-4% | 0%-1% |
| Ex 5090 | 4%-6% | 8%-10% | 4%-6% | 2%-4% | 1%-2% |
| Ex 5091 | 4%-6% | 8%-10% | 4%-6% | 2%-4% | 2%-4% |
| Ex 5092 | 4%-6% | 8%-10% | 4%-6% | 2%-4% | 4%-6% |
| Ex 5093 | 4%-6% | 8%-10% | 4%-6% | 2%-4% | 6%-8% |
| Ex 5094 | 4%-6% | 8%-10% | 4%-6% | 2%-4% | 8%-10% |
| Ex 5095 | 4%-6% | 8%-10% | 4%-6% | 4%-6% | 0%-1% |
| Ex 5096 | 4%-6% | 8%-10% | 4%-6% | 4%-6% | 1%-2% |
| Ex 5097 | 4%-6% | 8%-10% | 4%-6% | 4%-6% | 2%-4% |
| Ex 5098 | 4%-6% | 8%-10% | 4%-6% | 4%-6% | 4%-6% |
| Ex 5099 | 4%-6% | 8%-10% | 4%-6% | 4%-6% | 6%-8% |
| Ex 5100 | 4%-6% | 8%-10% | 4%-6% | 4%-6% | 8%-10% |
| Ex 5101 | 4%-6% | 8%-10% | 4%-6% | 6%-8% | 0%-1% |
| Ex 5102 | 4%-6% | 8%-10% | 4%-6% | 6%-8% | 1%-2% |
| Ex 5103 | 4%-6% | 8%-10% | 4%-6% | 6%-8% | 2%-4% |
| Ex 5104 | 4%-6% | 8%-10% | 4%-6% | 6%-8% | 4%-6% |
| Ex 5105 | 4%-6% | 8%-10% | 4%-6% | 6%-8% | 6%-8% |
| Ex 5106 | 4%-6% | 8%-10% | 4%-6% | 6%-8% | 8%-10% |
| Ex 5107 | 4%-6% | 8%-10% | 4%-6% | 8%-10% | 0%-1% |
| Ex 5108 | 4%-6% | 8%-10% | 4%-6% | 8%-10% | 1%-2% |
| Ex 5109 | 4%-6% | 8%-10% | 4%-6% | 8%-10% | 2%-4% |
| Ex 5110 | 4%-6% | 8%-10% | 4%-6% | 8%-10% | 4%-6% |
| Ex 5111 | 4%-6% | 8%-10% | 4%-6% | 8%-10% | 6%-8% |
| Ex 5112 | 4%-6% | 8%-10% | 4%-6% | 8%-10% | 8%-10% |
| Ex 5113 | 4%-6% | 8%-10% | 6%-8% | 0%-1% | 0%-1% |
| Ex 5114 | 4%-6% | 8%-10% | 6%-8% | 0%-1% | 1%-2% |
| Ex 5115 | 4%-6% | 8%-10% | 6%-8% | 0%-1% | 2%-4% |
| Ex 5116 | 4%-6% | 8%-10% | 6%-8% | 0%-1% | 4%-6% |
| Ex 5117 | 4%-6% | 8%-10% | 6%-8% | 0%-1% | 6%-8% |
| Ex 5118 | 4%-6% | 8%-10% | 6%-8% | 0%-1% | 8%-10% |
| Ex 5119 | 4%-6% | 8%-10% | 6%-8% | 1%-2% | 0%-1% |
| Ex 5120 | 4%-6% | 8%-10% | 6%-8% | 1%-2% | 1%-2% |
| Ex 5121 | 4%-6% | 8%-10% | 6%-8% | 1%-2% | 2%-4% |
| Ex 5122 | 4%-6% | 8%-10% | 6%-8% | 1%-2% | 4%-6% |
| Ex 5123 | 4%-6% | 8%-10% | 6%-8% | 1%-2% | 6%-8% |
| Ex 5124 | 4%-6% | 8%-10% | 6%-8% | 1%-2% | 8%-10% |
| Ex 5125 | 4%-6% | 8%-10% | 6%-8% | 2%-4% | 0%-1% |
| Ex 5126 | 4%-6% | 8%-10% | 6%-8% | 2%-4% | 1%-2% |
| Ex 5127 | 4%-6% | 8%-10% | 6%-8% | 2%-4% | 2%-4% |
| Ex 5128 | 4%-6% | 8%-10% | 6%-8% | 2%-4% | 4%-6% |
| Ex 5129 | 4%-6% | 8%-10% | 6%-8% | 2%-4% | 6%-8% |
| Ex 5130 | 4%-6% | 8%-10% | 6%-8% | 2%-4% | 8%-10% |
| Ex 5131 | 4%-6% | 8%-10% | 6%-8% | 4%-6% | 0%-1% |
| Ex 5132 | 4%-6% | 8%-10% | 6%-8% | 4%-6% | 1%-2% |
| Ex 5133 | 4%-6% | 8%-10% | 6%-8% | 4%-6% | 2%-4% |
| Ex 5134 | 4%-6% | 8%-10% | 6%-8% | 4%-6% | 4%-6% |
| Ex 5135 | 4%-6% | 8%-10% | 6%-8% | 4%-6% | 6%-8% |
| Ex 5136 | 4%-6% | 8%-10% | 6%-8% | 4%-6% | 8%-10% |
| Ex 5137 | 4%-6% | 8%-10% | 6%-8% | 6%-8% | 0%-1% |
| Ex 5138 | 4%-6% | 8%-10% | 6%-8% | 6%-8% | 1%-2% |
| Ex 5139 | 4%-6% | 8%-10% | 6%-8% | 6%-8% | 2%-4% |
| Ex 5140 | 4%-6% | 8%-10% | 6%-8% | 6%-8% | 4%-6% |
| Ex 5141 | 4%-6% | 8%-10% | 6%-8% | 6%-8% | 6%-8% |
| Ex 5142 | 4%-6% | 8%-10% | 6%-8% | 6%-8% | 8%-10% |
| Ex 5143 | 4%-6% | 8%-10% | 6%-8% | 8%-10% | 0%-1% |
| Ex 5144 | 4%-6% | 8%-10% | 6%-8% | 8%-10% | 1%-2% |
| Ex 5145 | 4%-6% | 8%-10% | 6%-8% | 8%-10% | 2%-4% |
| Ex 5146 | 4%-6% | 8%-10% | 6%-8% | 8%-10% | 4%-6% |
| Ex 5147 | 4%-6% | 8%-10% | 6%-8% | 8%-10% | 6%-8% |
| Ex 5148 | 4%-6% | 8%-10% | 6%-8% | 8%-10% | 8%-10% |
| Ex 5149 | 4%-6% | 8%-10% | 8%-10% | 0%-1% | 0%-1% |
| Ex 5150 | 4%-6% | 8%-10% | 8%-10% | 0%-1% | 1%-2% |
| Ex 5151 | 4%-6% | 8%-10% | 8%-10% | 0%-1% | 2%-4% |
| Ex 5152 | 4%-6% | 8%-10% | 8%-10% | 0%-1% | 4%-6% |
| Ex 5153 | 4%-6% | 8%-10% | 8%-10% | 0%-1% | 6%-8% |
| Ex 5154 | 4%-6% | 8%-10% | 8%-10% | 0%-1% | 8%-10% |
| Ex 5155 | 4%-6% | 8%-10% | 8%-10% | 1%-2% | 0%-1% |
| Ex 5156 | 4%-6% | 8%-10% | 8%-10% | 1%-2% | 1%-2% |
| Ex 5157 | 4%-6% | 8%-10% | 8%-10% | 1%-2% | 2%-4% |
| Ex 5158 | 4%-6% | 8%-10% | 8%-10% | 1%-2% | 4%-6% |
| Ex 5159 | 4%-6% | 8%-10% | 8%-10% | 1%-2% | 6%-8% |
| Ex 5160 | 4%-6% | 8%-10% | 8%-10% | 1%-2% | 8%-10% |
| Ex 5161 | 4%-6% | 8%-10% | 8%-10% | 2%-4% | 0%-1% |
| Ex 5162 | 4%-6% | 8%-10% | 8%-10% | 2%-4% | 1%-2% |
| Ex 5163 | 4%-6% | 8%-10% | 8%-10% | 2%-4% | 2%-4% |
| Ex 5164 | 4%-6% | 8%-10% | 8%-10% | 2%-4% | 4%-6% |
| Ex 5165 | 4%-6% | 8%-10% | 8%-10% | 2%-4% | 6%-8% |
| Ex 5166 | 4%-6% | 8%-10% | 8%-10% | 2%-4% | 8%-10% |
| Ex 5167 | 4%-6% | 8%-10% | 8%-10% | 4%-6% | 0%-1% |
| Ex 5168 | 4%-6% | 8%-10% | 8%-10% | 4%-6% | 1%-2% |
| Ex 5169 | 4%-6% | 8%-10% | 8%-10% | 4%-6% | 2%-4% |
| Ex 5170 | 4%-6% | 8%-10% | 8%-10% | 4%-6% | 4%-6% |
| Ex 5171 | 4%-6% | 8%-10% | 8%-10% | 4%-6% | 6%-8% |
| Ex 5172 | 4%-6% | 8%-10% | 8%-10% | 4%-6% | 8%-10% |
| Ex 5173 | 4%-6% | 8%-10% | 8%-10% | 6%-8% | 0%-1% |
| Ex 5174 | 4%-6% | 8%-10% | 8%-10% | 6%-8% | 1%-2% |
| Ex 5175 | 4%-6% | 8%-10% | 8%-10% | 6%-8% | 2%-4% |
| Ex 5176 | 4%-6% | 8%-10% | 8%-10% | 6%-8% | 4%-6% |
| Ex 5177 | 4%-6% | 8%-10% | 8%-10% | 6%-8% | 6%-8% |
| Ex 5178 | 4%-6% | 8%-10% | 8%-10% | 6%-8% | 8%-10% |
| Ex 5179 | 4%-6% | 8%-10% | 8%-10% | 8%-10% | 0%-1% |
| Ex 5180 | 4%-6% | 8%-10% | 8%-10% | 8%-10% | 1%-2% |
| Ex 5181 | 4%-6% | 8%-10% | 8%-10% | 8%-10% | 2%-4% |
| Ex 5182 | 4%-6% | 8%-10% | 8%-10% | 8%-10% | 4%-6% |
| Ex 5183 | 4%-6% | 8%-10% | 8%-10% | 8%-10% | 6%-8% |
| Ex 5184 | 4%-6% | 8%-10% | 8%-10% | 8%-10% | 8%-10% |
| Ex 5185 | 6%-8% | 0%-1% | 0%-1% | 0%-1% | 0%-1% |
| Ex 5186 | 6%-8% | 0%-1% | 0%-1% | 0%-1% | 1%-2% |
| Ex 5187 | 6%-8% | 0%-1% | 0%-1% | 0%-1% | 2%-4% |
| Ex 5188 | 6%-8% | 0%-1% | 0%-1% | 0%-1% | 4%-6% |
| Ex 5189 | 6%-8% | 0%-1% | 0%-1% | 0%-1% | 6%-8% |
| Ex 5190 | 6%-8% | 0%-1% | 0%-1% | 0%-1% | 8%-10% |
| Ex 5191 | 6%-8% | 0%-1% | 0%-1% | 1%-2% | 0%-1% |
| Ex 5192 | 6%-8% | 0%-1% | 0%-1% | 1%-2% | 1%-2% |
| Ex 5193 | 6%-8% | 0%-1% | 0%-1% | 1%-2% | 2%-4% |
| Ex 5194 | 6%-8% | 0%-1% | 0%-1% | 1%-2% | 4%-6% |
| Ex 5195 | 6%-8% | 0%-1% | 0%-1% | 1%-2% | 6%-8% |
| Ex 5196 | 6%-8% | 0%-1% | 0%-1% | 1%-2% | 8%-10% |
| Ex 5197 | 6%-8% | 0%-1% | 0%-1% | 2%-4% | 0%-1% |
| Ex 5198 | 6%-8% | 0%-1% | 0%-1% | 2%-4% | 1%-2% |
| Ex 5199 | 6%-8% | 0%-1% | 0%-1% | 2%-4% | 2%-4% |
| Ex 5200 | 6%-8% | 0%-1% | 0%-1% | 2%-4% | 4%-6% |
| Ex 5201 | 6%-8% | 0%-1% | 0%-1% | 2%-4% | 6%-8% |
| Ex 5202 | 6%-8% | 0%-1% | 0%-1% | 2%-4% | 8%-10% |
| Ex 5203 | 6%-8% | 0%-1% | 0%-1% | 4%-6% | 0%-1% |
| Ex 5204 | 6%-8% | 0%-1% | 0%-1% | 4%-6% | 1%-2% |
| Ex 5205 | 6%-8% | 0%-1% | 0%-1% | 4%-6% | 2%-4% |
| Ex 5206 | 6%-8% | 0%-1% | 0%-1% | 4%-6% | 4%-6% |
| Ex 5207 | 6%-8% | 0%-1% | 0%-1% | 4%-6% | 6%-8% |
| Ex 5208 | 6%-8% | 0%-1% | 0%-1% | 4%-6% | 8%-10% |
| Ex 5209 | 6%-8% | 0%-1% | 0%-1% | 6%-8% | 0%-1% |
| Ex 5210 | 6%-8% | 0%-1% | 0%-1% | 6%-8% | 1%-2% |
| Ex 5211 | 6%-8% | 0%-1% | 0%-1% | 6%-8% | 2%-4% |
| Ex 5212 | 6%-8% | 0%-1% | 0%-1% | 6%-8% | 4%-6% |
| Ex 5213 | 6%-8% | 0%-1% | 0%-1% | 6%-8% | 6%-8% |
| Ex 5214 | 6%-8% | 0%-1% | 0%-1% | 6%-8% | 8%-10% |
| Ex 5215 | 6%-8% | 0%-1% | 0%-1% | 8%-10% | 0%-1% |
| Ex 5216 | 6%-8% | 0%-1% | 0%-1% | 8%-10% | 1%-2% |
| Ex 5217 | 6%-8% | 0%-1% | 0%-1% | 8%-10% | 2%-4% |
| Ex 5218 | 6%-8% | 0%-1% | 0%-1% | 8%-10% | 4%-6% |
| Ex 5219 | 6%-8% | 0%-1% | 0%-1% | 8%-10% | 6%-8% |
| Ex 5220 | 6%-8% | 0%-1% | 0%-1% | 8%-10% | 8%-10% |
| Ex 5221 | 6%-8% | 0%-1% | 1%-2% | 0%-1% | 0%-1% |
| Ex 5222 | 6%-8% | 0%-1% | 1%-2% | 0%-1% | 1%-2% |
| Ex 5223 | 6%-8% | 0%-1% | 1%-2% | 0%-1% | 2%-4% |
| Ex 5224 | 6%-8% | 0%-1% | 1%-2% | 0%-1% | 4%-6% |
| Ex 5225 | 6%-8% | 0%-1% | 1%-2% | 0%-1% | 6%-8% |
| Ex 5226 | 6%-8% | 0%-1% | 1%-2% | 0%-1% | 8%-10% |
| Ex 5227 | 6%-8% | 0%-1% | 1%-2% | 1%-2% | 0%-1% |
| Ex 5228 | 6%-8% | 0%-1% | 1%-2% | 1%-2% | 1%-2% |
| Ex 5229 | 6%-8% | 0%-1% | 1%-2% | 1%-2% | 2%-4% |
| Ex 5230 | 6%-8% | 0%-1% | 1%-2% | 1%-2% | 4%-6% |
| Ex 5231 | 6%-8% | 0%-1% | 1%-2% | 1%-2% | 6%-8% |
| Ex 5232 | 6%-8% | 0%-1% | 1%-2% | 1%-2% | 8%-10% |
| Ex 5233 | 6%-8% | 0%-1% | 1%-2% | 2%-4% | 0%-1% |
| Ex 5234 | 6%-8% | 0%-1% | 1%-2% | 2%-4% | 1%-2% |
| Ex 5235 | 6%-8% | 0%-1% | 1%-2% | 2%-4% | 2%-4% |
| Ex 5236 | 6%-8% | 0%-1% | 1%-2% | 2%-4% | 4%-6% |
| Ex 5237 | 6%-8% | 0%-1% | 1%-2% | 2%-4% | 6%-8% |
| Ex 5238 | 6%-8% | 0%-1% | 1%-2% | 2%-4% | 8%-10% |
| Ex 5239 | 6%-8% | 0%-1% | 1%-2% | 4%-6% | 0%-1% |
| Ex 5240 | 6%-8% | 0%-1% | 1%-2% | 4%-6% | 1%-2% |
| Ex 5241 | 6%-8% | 0%-1% | 1%-2% | 4%-6% | 2%-4% |
| Ex 5242 | 6%-8% | 0%-1% | 1%-2% | 4%-6% | 4%-6% |
| Ex 5243 | 6%-8% | 0%-1% | 1%-2% | 4%-6% | 6%-8% |
| Ex 5244 | 6%-8% | 0%-1% | 1%-2% | 4%-6% | 8%-10% |
| Ex 5245 | 6%-8% | 0%-1% | 1%-2% | 6%-8% | 0%-1% |
| Ex 5246 | 6%-8% | 0%-1% | 1%-2% | 6%-8% | 1%-2% |
| Ex 5247 | 6%-8% | 0%-1% | 1%-2% | 6%-8% | 2%-4% |
| Ex 5248 | 6%-8% | 0%-1% | 1%-2% | 6%-8% | 4%-6% |
| Ex 5249 | 6%-8% | 0%-1% | 1%-2% | 6%-8% | 6%-8% |
| Ex 5250 | 6%-8% | 0%-1% | 1%-2% | 6%-8% | 8%-10% |
| Ex 5251 | 6%-8% | 0%-1% | 1%-2% | 8%-10% | 0%-1% |
| Ex 5252 | 6%-8% | 0%-1% | 1%-2% | 8%-10% | 1%-2% |
| Ex 5253 | 6%-8% | 0%-1% | 1%-2% | 8%-10% | 2%-4% |
| Ex 5254 | 6%-8% | 0%-1% | 1%-2% | 8%-10% | 4%-6% |
| Ex 5255 | 6%-8% | 0%-1% | 1%-2% | 8%-10% | 6%-8% |
| Ex 5256 | 6%-8% | 0%-1% | 1%-2% | 8%-10% | 8%-10% |
| Ex 5257 | 6%-8% | 0%-1% | 2%-4% | 0%-1% | 0%-1% |
| Ex 5258 | 6%-8% | 0%-1% | 2%-4% | 0%-1% | 1%-2% |
| Ex 5259 | 6%-8% | 0%-1% | 2%-4% | 0%-1% | 2%-4% |
| Ex 5260 | 6%-8% | 0%-1% | 2%-4% | 0%-1% | 4%-6% |
| Ex 5261 | 6%-8% | 0%-1% | 2%-4% | 0%-1% | 6%-8% |
| Ex 5262 | 6%-8% | 0%-1% | 2%-4% | 0%-1% | 8%-10% |
| Ex 5263 | 6%-8% | 0%-1% | 2%-4% | 1%-2% | 0%-1% |
| Ex 5264 | 6%-8% | 0%-1% | 2%-4% | 1%-2% | 1%-2% |
| Ex 5265 | 6%-8% | 0%-1% | 2%-4% | 1%-2% | 2%-4% |
| Ex 5266 | 6%-8% | 0%-1% | 2%-4% | 1%-2% | 4%-6% |
| Ex 5267 | 6%-8% | 0%-1% | 2%-4% | 1%-2% | 6%-8% |
| Ex 5268 | 6%-8% | 0%-1% | 2%-4% | 1%-2% | 8%-10% |
| Ex 5269 | 6%-8% | 0%-1% | 2%-4% | 2%-4% | 0%-1% |
| Ex 5270 | 6%-8% | 0%-1% | 2%-4% | 2%-4% | 1%-2% |
| Ex 5271 | 6%-8% | 0%-1% | 2%-4% | 2%-4% | 2%-4% |
| Ex 5272 | 6%-8% | 0%-1% | 2%-4% | 2%-4% | 4%-6% |
| Ex 5273 | 6%-8% | 0%-1% | 2%-4% | 2%-4% | 6%-8% |
| Ex 5274 | 6%-8% | 0%-1% | 2%-4% | 2%-4% | 8%-10% |
| Ex 5275 | 6%-8% | 0%-1% | 2%-4% | 4%-6% | 0%-1% |
| Ex 5276 | 6%-8% | 0%-1% | 2%-4% | 4%-6% | 1%-2% |
| Ex 5277 | 6%-8% | 0%-1% | 2%-4% | 4%-6% | 2%-4% |
| Ex 5278 | 6%-8% | 0%-1% | 2%-4% | 4%-6% | 4%-6% |
| Ex 5279 | 6%-8% | 0%-1% | 2%-4% | 4%-6% | 6%-8% |
| Ex 5280 | 6%-8% | 0%-1% | 2%-4% | 4%-6% | 8%-10% |
| Ex 5281 | 6%-8% | 0%-1% | 2%-4% | 6%-8% | 0%-1% |
| Ex 5282 | 6%-8% | 0%-1% | 2%-4% | 6%-8% | 1%-2% |
| Ex 5283 | 6%-8% | 0%-1% | 2%-4% | 6%-8% | 2%-4% |
| Ex 5284 | 6%-8% | 0%-1% | 2%-4% | 6%-8% | 4%-6% |
| Ex 5285 | 6%-8% | 0%-1% | 2%-4% | 6%-8% | 6%-8% |
| Ex 5286 | 6%-8% | 0%-1% | 2%-4% | 6%-8% | 8%-10% |
| Ex 5287 | 6%-8% | 0%-1% | 2%-4% | 8%-10% | 0%-1% |
| Ex 5288 | 6%-8% | 0%-1% | 2%-4% | 8%-10% | 1%-2% |
| Ex 5289 | 6%-8% | 0%-1% | 2%-4% | 8%-10% | 2%-4% |
| Ex 5290 | 6%-8% | 0%-1% | 2%-4% | 8%-10% | 4%-6% |
| Ex 5291 | 6%-8% | 0%-1% | 2%-4% | 8%-10% | 6%-8% |
| Ex 5292 | 6%-8% | 0%-1% | 2%-4% | 8%-10% | 8%-10% |
| Ex 5293 | 6%-8% | 0%-1% | 4%-6% | 0%-1% | 0%-1% |
| Ex 5294 | 6%-8% | 0%-1% | 4%-6% | 0%-1% | 1%-2% |
| Ex 5295 | 6%-8% | 0%-1% | 4%-6% | 0%-1% | 2%-4% |
| Ex 5296 | 6%-8% | 0%-1% | 4%-6% | 0%-1% | 4%-6% |
| Ex 5297 | 6%-8% | 0%-1% | 4%-6% | 0%-1% | 6%-8% |
| Ex 5298 | 6%-8% | 0%-1% | 4%-6% | 0%-1% | 8%-10% |
| Ex 5299 | 6%-8% | 0%-1% | 4%-6% | 1%-2% | 0%-1% |
| Ex 5300 | 6%-8% | 0%-1% | 4%-6% | 1%-2% | 1%-2% |
| Ex 5301 | 6%-8% | 0%-1% | 4%-6% | 1%-2% | 2%-4% |
| Ex 5302 | 6%-8% | 0%-1% | 4%-6% | 1%-2% | 4%-6% |
| Ex 5303 | 6%-8% | 0%-1% | 4%-6% | 1%-2% | 6%-8% |
| Ex 5304 | 6%-8% | 0%-1% | 4%-6% | 1%-2% | 8%-10% |
| Ex 5305 | 6%-8% | 0%-1% | 4%-6% | 2%-4% | 0%-1% |
| Ex 5306 | 6%-8% | 0%-1% | 4%-6% | 2%-4% | 1%-2% |
| Ex 5307 | 6%-8% | 0%-1% | 4%-6% | 2%-4% | 2%-4% |
| Ex 5308 | 6%-8% | 0%-1% | 4%-6% | 2%-4% | 4%-6% |
| Ex 5309 | 6%-8% | 0%-1% | 4%-6% | 2%-4% | 6%-8% |
| Ex 5310 | 6%-8% | 0%-1% | 4%-6% | 2%-4% | 8%-10% |
| Ex 5311 | 6%-8% | 0%-1% | 4%-6% | 4%-6% | 0%-1% |
| Ex 5312 | 6%-8% | 0%-1% | 4%-6% | 4%-6% | 1%-2% |
| Ex 5313 | 6%-8% | 0%-1% | 4%-6% | 4%-6% | 2%-4% |
| Ex 5314 | 6%-8% | 0%-1% | 4%-6% | 4%-6% | 4%-6% |
| Ex 5315 | 6%-8% | 0%-1% | 4%-6% | 4%-6% | 6%-8% |
| Ex 5316 | 6%-8% | 0%-1% | 4%-6% | 4%-6% | 8%-10% |
| Ex 5317 | 6%-8% | 0%-1% | 4%-6% | 6%-8% | 0%-1% |
| Ex 5318 | 6%-8% | 0%-1% | 4%-6% | 6%-8% | 1%-2% |
| Ex 5319 | 6%-8% | 0%-1% | 4%-6% | 6%-8% | 2%-4% |
| Ex 5320 | 6%-8% | 0%-1% | 4%-6% | 6%-8% | 4%-6% |
| Ex 5321 | 6%-8% | 0%-1% | 4%-6% | 6%-8% | 6%-8% |
| Ex 5322 | 6%-8% | 0%-1% | 4%-6% | 6%-8% | 8%-10% |
| Ex 5323 | 6%-8% | 0%-1% | 4%-6% | 8%-10% | 0%-1% |
| Ex 5324 | 6%-8% | 0%-1% | 4%-6% | 8%-10% | 1%-2% |
| Ex 5325 | 6%-8% | 0%-1% | 4%-6% | 8%-10% | 2%-4% |
| Ex 5326 | 6%-8% | 0%-1% | 4%-6% | 8%-10% | 4%-6% |
| Ex 5327 | 6%-8% | 0%-1% | 4%-6% | 8%-10% | 6%-8% |
| Ex 5328 | 6%-8% | 0%-1% | 4%-6% | 8%-10% | 8%-10% |
| Ex 5329 | 6%-8% | 0%-1% | 6%-8% | 0%-1% | 0%-1% |
| Ex 5330 | 6%-8% | 0%-1% | 6%-8% | 0%-1% | 1%-2% |
| Ex 5331 | 6%-8% | 0%-1% | 6%-8% | 0%-1% | 2%-4% |
| Ex 5332 | 6%-8% | 0%-1% | 6%-8% | 0%-1% | 4%-6% |
| Ex 5333 | 6%-8% | 0%-1% | 6%-8% | 0%-1% | 6%-8% |
| Ex 5334 | 6%-8% | 0%-1% | 6%-8% | 0%-1% | 8%-10% |
| Ex 5335 | 6%-8% | 0%-1% | 6%-8% | 1%-2% | 0%-1% |
| Ex 5336 | 6%-8% | 0%-1% | 6%-8% | 1%-2% | 1%-2% |
| Ex 5337 | 6%-8% | 0%-1% | 6%-8% | 1%-2% | 2%-4% |
| Ex 5338 | 6%-8% | 0%-1% | 6%-8% | 1%-2% | 4%-6% |
| Ex 5339 | 6%-8% | 0%-1% | 6%-8% | 1%-2% | 6%-8% |
| Ex 5340 | 6%-8% | 0%-1% | 6%-8% | 1%-2% | 8%-10% |
| Ex 5341 | 6%-8% | 0%-1% | 6%-8% | 2%-4% | 0%-1% |
| Ex 5342 | 6%-8% | 0%-1% | 6%-8% | 2%-4% | 1%-2% |
| Ex 5343 | 6%-8% | 0%-1% | 6%-8% | 2%-4% | 2%-4% |
| Ex 5344 | 6%-8% | 0%-1% | 6%-8% | 2%-4% | 4%-6% |
| Ex 5345 | 6%-8% | 0%-1% | 6%-8% | 2%-4% | 6%-8% |
| Ex 5346 | 6%-8% | 0%-1% | 6%-8% | 2%-4% | 8%-10% |
| Ex 5347 | 6%-8% | 0%-1% | 6%-8% | 4%-6% | 0%-1% |
| Ex 5348 | 6%-8% | 0%-1% | 6%-8% | 4%-6% | 1%-2% |
| Ex 5349 | 6%-8% | 0%-1% | 6%-8% | 4%-6% | 2%-4% |
| Ex 5350 | 6%-8% | 0%-1% | 6%-8% | 4%-6% | 4%-6% |
| Ex 5351 | 6%-8% | 0%-1% | 6%-8% | 4%-6% | 6%-8% |
| Ex 5352 | 6%-8% | 0%-1% | 6%-8% | 4%-6% | 8%-10% |
| Ex 5353 | 6%-8% | 0%-1% | 6%-8% | 6%-8% | 0%-1% |
| Ex 5354 | 6%-8% | 0%-1% | 6%-8% | 6%-8% | 1%-2% |
| Ex 5355 | 6%-8% | 0%-1% | 6%-8% | 6%-8% | 2%-4% |
| Ex 5356 | 6%-8% | 0%-1% | 6%-8% | 6%-8% | 4%-6% |
| Ex 5357 | 6%-8% | 0%-1% | 6%-8% | 6%-8% | 6%-8% |
| Ex 5358 | 6%-8% | 0%-1% | 6%-8% | 6%-8% | 8%-10% |
| Ex 5359 | 6%-8% | 0%-1% | 6%-8% | 8%-10% | 0%-1% |
| Ex 5360 | 6%-8% | 0%-1% | 6%-8% | 8%-10% | 1%-2% |
| Ex 5361 | 6%-8% | 0%-1% | 6%-8% | 8%-10% | 2%-4% |
| Ex 5362 | 6%-8% | 0%-1% | 6%-8% | 8%-10% | 4%-6% |
| Ex 5363 | 6%-8% | 0%-1% | 6%-8% | 8%-10% | 6%-8% |
| Ex 5364 | 6%-8% | 0%-1% | 6%-8% | 8%-10% | 8%-10% |
| Ex 5365 | 6%-8% | 0%-1% | 8%-10% | 0%-1% | 0%-1% |
| Ex 5366 | 6%-8% | 0%-1% | 8%-10% | 0%-1% | 1%-2% |
| Ex 5367 | 6%-8% | 0%-1% | 8%-10% | 0%-1% | 2%-4% |
| Ex 5368 | 6%-8% | 0%-1% | 8%-10% | 0%-1% | 4%-6% |
| Ex 5369 | 6%-8% | 0%-1% | 8%-10% | 0%-1% | 6%-8% |
| Ex 5370 | 6%-8% | 0%-1% | 8%-10% | 0%-1% | 8%-10% |
| Ex 5371 | 6%-8% | 0%-1% | 8%-10% | 1%-2% | 0%-1% |
| Ex 5372 | 6%-8% | 0%-1% | 8%-10% | 1%-2% | 1%-2% |
| Ex 5373 | 6%-8% | 0%-1% | 8%-10% | 1%-2% | 2%-4% |
| Ex 5374 | 6%-8% | 0%-1% | 8%-10% | 1%-2% | 4%-6% |
| Ex 5375 | 6%-8% | 0%-1% | 8%-10% | 1%-2% | 6%-8% |
| Ex 5376 | 6%-8% | 0%-1% | 8%-10% | 1%-2% | 8%-10% |
| Ex 5377 | 6%-8% | 0%-1% | 8%-10% | 2%-4% | 0%-1% |
| Ex 5378 | 6%-8% | 0%-1% | 8%-10% | 2%-4% | 1%-2% |
| Ex 5379 | 6%-8% | 0%-1% | 8%-10% | 2%-4% | 2%-4% |
| Ex 5380 | 6%-8% | 0%-1% | 8%-10% | 2%-4% | 4%-6% |
| Ex 5381 | 6%-8% | 0%-1% | 8%-10% | 2%-4% | 6%-8% |
| Ex 5382 | 6%-8% | 0%-1% | 8%-10% | 2%-4% | 8%-10% |
| Ex 5383 | 6%-8% | 0%-1% | 8%-10% | 4%-6% | 0%-1% |
| Ex 5384 | 6%-8% | 0%-1% | 8%-10% | 4%-6% | 1%-2% |
| Ex 5385 | 6%-8% | 0%-1% | 8%-10% | 4%-6% | 2%-4% |
| Ex 5386 | 6%-8% | 0%-1% | 8%-10% | 4%-6% | 4%-6% |
| Ex 5387 | 6%-8% | 0%-1% | 8%-10% | 4%-6% | 6%-8% |
| Ex 5388 | 6%-8% | 0%-1% | 8%-10% | 4%-6% | 8%-10% |
| Ex 5389 | 6%-8% | 0%-1% | 8%-10% | 6%-8% | 0%-1% |
| Ex 5390 | 6%-8% | 0%-1% | 8%-10% | 6%-8% | 1%-2% |
| Ex 5391 | 6%-8% | 0%-1% | 8%-10% | 6%-8% | 2%-4% |
| Ex 5392 | 6%-8% | 0%-1% | 8%-10% | 6%-8% | 4%-6% |
| Ex 5393 | 6%-8% | 0%-1% | 8%-10% | 6%-8% | 6%-8% |
| Ex 5394 | 6%-8% | 0%-1% | 8%-10% | 6%-8% | 8%-10% |
| Ex 5395 | 6%-8% | 0%-1% | 8%-10% | 8%-10% | 0%-1% |
| Ex 5396 | 6%-8% | 0%-1% | 8%-10% | 8%-10% | 1%-2% |
| Ex 5397 | 6%-8% | 0%-1% | 8%-10% | 8%-10% | 2%-4% |
| Ex 5398 | 6%-8% | 0%-1% | 8%-10% | 8%-10% | 4%-6% |
| Ex 5399 | 6%-8% | 0%-1% | 8%-10% | 8%-10% | 6%-8% |
| Ex 5400 | 6%-8% | 0%-1% | 8%-10% | 8%-10% | 8%-10% |
| Ex 5401 | 6%-8% | 1%-2% | 0%-1% | 0%-1% | 0%-1% |
| Ex 5402 | 6%-8% | 1%-2% | 0%-1% | 0%-1% | 1%-2% |
| Ex 5403 | 6%-8% | 1%-2% | 0%-1% | 0%-1% | 2%-4% |
| Ex 5404 | 6%-8% | 1%-2% | 0%-1% | 0%-1% | 4%-6% |
| Ex 5405 | 6%-8% | 1%-2% | 0%-1% | 0%-1% | 6%-8% |
| Ex 5406 | 6%-8% | 1%-2% | 0%-1% | 0%-1% | 8%-10% |
| Ex 5407 | 6%-8% | 1%-2% | 0%-1% | 1%-2% | 0%-1% |
| Ex 5408 | 6%-8% | 1%-2% | 0%-1% | 1%-2% | 1%-2% |
| Ex 5409 | 6%-8% | 1%-2% | 0%-1% | 1%-2% | 2%-4% |
| Ex 5410 | 6%-8% | 1%-2% | 0%-1% | 1%-2% | 4%-6% |
| Ex 5411 | 6%-8% | 1%-2% | 0%-1% | 1%-2% | 6%-8% |
| Ex 5412 | 6%-8% | 1%-2% | 0%-1% | 1%-2% | 8%-10% |
| Ex 5413 | 6%-8% | 1%-2% | 0%-1% | 2%-4% | 0%-1% |
| Ex 5414 | 6%-8% | 1%-2% | 0%-1% | 2%-4% | 1%-2% |
| Ex 5415 | 6%-8% | 1%-2% | 0%-1% | 2%-4% | 2%-4% |
| Ex 5416 | 6%-8% | 1%-2% | 0%-1% | 2%-4% | 4%-6% |
| Ex 5417 | 6%-8% | 1%-2% | 0%-1% | 2%-4% | 6%-8% |
| Ex 5418 | 6%-8% | 1%-2% | 0%-1% | 2%-4% | 8%-10% |
| Ex 5419 | 6%-8% | 1%-2% | 0%-1% | 4%-6% | 0%-1% |
| Ex 5420 | 6%-8% | 1%-2% | 0%-1% | 4%-6% | 1%-2% |
| Ex 5421 | 6%-8% | 1%-2% | 0%-1% | 4%-6% | 2%-4% |
| Ex 5422 | 6%-8% | 1%-2% | 0%-1% | 4%-6% | 4%-6% |
| Ex 5423 | 6%-8% | 1%-2% | 0%-1% | 4%-6% | 6%-8% |
| Ex 5424 | 6%-8% | 1%-2% | 0%-1% | 4%-6% | 8%-10% |
| Ex 5425 | 6%-8% | 1%-2% | 0%-1% | 6%-8% | 0%-1% |
| Ex 5426 | 6%-8% | 1%-2% | 0%-1% | 6%-8% | 1%-2% |
| Ex 5427 | 6%-8% | 1%-2% | 0%-1% | 6%-8% | 2%-4% |
| Ex 5428 | 6%-8% | 1%-2% | 0%-1% | 6%-8% | 4%-6% |
| Ex 5429 | 6%-8% | 1%-2% | 0%-1% | 6%-8% | 6%-8% |
| Ex 5430 | 6%-8% | 1%-2% | 0%-1% | 6%-8% | 8%-10% |
| Ex 5431 | 6%-8% | 1%-2% | 0%-1% | 8%-10% | 0%-1% |
| Ex 5432 | 6%-8% | 1%-2% | 0%-1% | 8%-10% | 1%-2% |
| Ex 5433 | 6%-8% | 1%-2% | 0%-1% | 8%-10% | 2%-4% |
| Ex 5434 | 6%-8% | 1%-2% | 0%-1% | 8%-10% | 4%-6% |
| Ex 5435 | 6%-8% | 1%-2% | 0%-1% | 8%-10% | 6%-8% |
| Ex 5436 | 6%-8% | 1%-2% | 0%-1% | 8%-10% | 8%-10% |
| Ex 5437 | 6%-8% | 1%-2% | 1%-2% | 0%-1% | 0%-1% |
| Ex 5438 | 6%-8% | 1%-2% | 1%-2% | 0%-1% | 1%-2% |
| Ex 5439 | 6%-8% | 1%-2% | 1%-2% | 0%-1% | 2%-4% |
| Ex 5440 | 6%-8% | 1%-2% | 1%-2% | 0%-1% | 4%-6% |
| Ex 5441 | 6%-8% | 1%-2% | 1%-2% | 0%-1% | 6%-8% |
| Ex 5442 | 6%-8% | 1%-2% | 1%-2% | 0%-1% | 8%-10% |
| Ex 5443 | 6%-8% | 1%-2% | 1%-2% | 1%-2% | 0%-1% |
| Ex 5444 | 6%-8% | 1%-2% | 1%-2% | 1%-2% | 1%-2% |
| Ex 5445 | 6%-8% | 1%-2% | 1%-2% | 1%-2% | 2%-4% |
| Ex 5446 | 6%-8% | 1%-2% | 1%-2% | 1%-2% | 4%-6% |
| Ex 5447 | 6%-8% | 1%-2% | 1%-2% | 1%-2% | 6%-8% |
| Ex 5448 | 6%-8% | 1%-2% | 1%-2% | 1%-2% | 8%-10% |
| Ex 5449 | 6%-8% | 1%-2% | 1%-2% | 2%-4% | 0%-1% |
| Ex 5450 | 6%-8% | 1%-2% | 1%-2% | 2%-4% | 1%-2% |
| Ex 5451 | 6%-8% | 1%-2% | 1%-2% | 2%-4% | 2%-4% |
| Ex 5452 | 6%-8% | 1%-2% | 1%-2% | 2%-4% | 4%-6% |
| Ex 5453 | 6%-8% | 1%-2% | 1%-2% | 2%-4% | 6%-8% |
| Ex 5454 | 6%-8% | 1%-2% | 1%-2% | 2%-4% | 8%-10% |
| Ex 5455 | 6%-8% | 1%-2% | 1%-2% | 4%-6% | 0%-1% |
| Ex 5456 | 6%-8% | 1%-2% | 1%-2% | 4%-6% | 1%-2% |
| Ex 5457 | 6%-8% | 1%-2% | 1%-2% | 4%-6% | 2%-4% |
| Ex 5458 | 6%-8% | 1%-2% | 1%-2% | 4%-6% | 4%-6% |
| Ex 5459 | 6%-8% | 1%-2% | 1%-2% | 4%-6% | 6%-8% |
| Ex 5460 | 6%-8% | 1%-2% | 1%-2% | 4%-6% | 8%-10% |
| Ex 5461 | 6%-8% | 1%-2% | 1%-2% | 6%-8% | 0%-1% |
| Ex 5462 | 6%-8% | 1%-2% | 1%-2% | 6%-8% | 1%-2% |
| Ex 5463 | 6%-8% | 1%-2% | 1%-2% | 6%-8% | 2%-4% |
| Ex 5464 | 6%-8% | 1%-2% | 1%-2% | 6%-8% | 4%-6% |
| Ex 5465 | 6%-8% | 1%-2% | 1%-2% | 6%-8% | 6%-8% |
| Ex 5466 | 6%-8% | 1%-2% | 1%-2% | 6%-8% | 8%-10% |
| Ex 5467 | 6%-8% | 1%-2% | 1%-2% | 8%-10% | 0%-1% |
| Ex 5468 | 6%-8% | 1%-2% | 1%-2% | 8%-10% | 1%-2% |
| Ex 5469 | 6%-8% | 1%-2% | 1%-2% | 8%-10% | 2%-4% |
| Ex 5470 | 6%-8% | 1%-2% | 1%-2% | 8%-10% | 4%-6% |
| Ex 5471 | 6%-8% | 1%-2% | 1%-2% | 8%-10% | 6%-8% |
| Ex 5472 | 6%-8% | 1%-2% | 1%-2% | 8%-10% | 8%-10% |
| Ex 5473 | 6%-8% | 1%-2% | 2%-4% | 0%-1% | 0%-1% |
| Ex 5474 | 6%-8% | 1%-2% | 2%-4% | 0%-1% | 1%-2% |
| Ex 5475 | 6%-8% | 1%-2% | 2%-4% | 0%-1% | 2%-4% |
| Ex 5476 | 6%-8% | 1%-2% | 2%-4% | 0%-1% | 4%-6% |
| Ex 5477 | 6%-8% | 1%-2% | 2%-4% | 0%-1% | 6%-8% |
| Ex 5478 | 6%-8% | 1%-2% | 2%-4% | 0%-1% | 8%-10% |
| Ex 5479 | 6%-8% | 1%-2% | 2%-4% | 1%-2% | 0%-1% |
| Ex 5480 | 6%-8% | 1%-2% | 2%-4% | 1%-2% | 1%-2% |
| Ex 5481 | 6%-8% | 1%-2% | 2%-4% | 1%-2% | 2%-4% |
| Ex 5482 | 6%-8% | 1%-2% | 2%-4% | 1%-2% | 4%-6% |
| Ex 5483 | 6%-8% | 1%-2% | 2%-4% | 1%-2% | 6%-8% |
| Ex 5484 | 6%-8% | 1%-2% | 2%-4% | 1%-2% | 8%-10% |
| Ex 5485 | 6%-8% | 1%-2% | 2%-4% | 2%-4% | 0%-1% |
| Ex 5486 | 6%-8% | 1%-2% | 2%-4% | 2%-4% | 1%-2% |
| Ex 5487 | 6%-8% | 1%-2% | 2%-4% | 2%-4% | 2%-4% |
| Ex 5488 | 6%-8% | 1%-2% | 2%-4% | 2%-4% | 4%-6% |
| Ex 5489 | 6%-8% | 1%-2% | 2%-4% | 2%-4% | 6%-8% |
| Ex 5490 | 6%-8% | 1%-2% | 2%-4% | 2%-4% | 8%-10% |
| Ex 5491 | 6%-8% | 1%-2% | 2%-4% | 4%-6% | 0%-1% |
| Ex 5492 | 6%-8% | 1%-2% | 2%-4% | 4%-6% | 1%-2% |
| Ex 5493 | 6%-8% | 1%-2% | 2%-4% | 4%-6% | 2%-4% |
| Ex 5494 | 6%-8% | 1%-2% | 2%-4% | 4%-6% | 4%-6% |
| Ex 5495 | 6%-8% | 1%-2% | 2%-4% | 4%-6% | 6%-8% |
| Ex 5496 | 6%-8% | 1%-2% | 2%-4% | 4%-6% | 8%-10% |
| Ex 5497 | 6%-8% | 1%-2% | 2%-4% | 6%-8% | 0%-1% |
| Ex 5498 | 6%-8% | 1%-2% | 2%-4% | 6%-8% | 1%-2% |
| Ex 5499 | 6%-8% | 1%-2% | 2%-4% | 6%-8% | 2%-4% |
| Ex 5500 | 6%-8% | 1%-2% | 2%-4% | 6%-8% | 4%-6% |
| Ex 5501 | 6%-8% | 1%-2% | 2%-4% | 6%-8% | 6%-8% |
| Ex 5502 | 6%-8% | 1%-2% | 2%-4% | 6%-8% | 8%-10% |
| Ex 5503 | 6%-8% | 1%-2% | 2%-4% | 8%-10% | 0%-1% |
| Ex 5504 | 6%-8% | 1%-2% | 2%-4% | 8%-10% | 1%-2% |
| Ex 5505 | 6%-8% | 1%-2% | 2%-4% | 8%-10% | 2%-4% |
| Ex 5506 | 6%-8% | 1%-2% | 2%-4% | 8%-10% | 4%-6% |
| Ex 5507 | 6%-8% | 1%-2% | 2%-4% | 8%-10% | 6%-8% |
| Ex 5508 | 6%-8% | 1%-2% | 2%-4% | 8%-10% | 8%-10% |
| Ex 5509 | 6%-8% | 1%-2% | 4%-6% | 0%-1% | 0%-1% |
| Ex 5510 | 6%-8% | 1%-2% | 4%-6% | 0%-1% | 1%-2% |
| Ex 5511 | 6%-8% | 1%-2% | 4%-6% | 0%-1% | 2%-4% |
| Ex 5512 | 6%-8% | 1%-2% | 4%-6% | 0%-1% | 4%-6% |
| Ex 5513 | 6%-8% | 1%-2% | 4%-6% | 0%-1% | 6%-8% |
| Ex 5514 | 6%-8% | 1%-2% | 4%-6% | 0%-1% | 8%-10% |
| Ex 5515 | 6%-8% | 1%-2% | 4%-6% | 1%-2% | 0%-1% |
| Ex 5516 | 6%-8% | 1%-2% | 4%-6% | 1%-2% | 1%-2% |
| Ex 5517 | 6%-8% | 1%-2% | 4%-6% | 1%-2% | 2%-4% |
| Ex 5518 | 6%-8% | 1%-2% | 4%-6% | 1%-2% | 4%-6% |
| Ex 5519 | 6%-8% | 1%-2% | 4%-6% | 1%-2% | 6%-8% |
| Ex 5520 | 6%-8% | 1%-2% | 4%-6% | 1%-2% | 8%-10% |
| Ex 5521 | 6%-8% | 1%-2% | 4%-6% | 2%-4% | 0%-1% |
| Ex 5522 | 6%-8% | 1%-2% | 4%-6% | 2%-4% | 1%-2% |
| Ex 5523 | 6%-8% | 1%-2% | 4%-6% | 2%-4% | 2%-4% |
| Ex 5524 | 6%-8% | 1%-2% | 4%-6% | 2%-4% | 4%-6% |
| Ex 5525 | 6%-8% | 1%-2% | 4%-6% | 2%-4% | 6%-8% |
| Ex 5526 | 6%-8% | 1%-2% | 4%-6% | 2%-4% | 8%-10% |
| Ex 5527 | 6%-8% | 1%-2% | 4%-6% | 4%-6% | 0%-1% |
| Ex 5528 | 6%-8% | 1%-2% | 4%-6% | 4%-6% | 1%-2% |
| Ex 5529 | 6%-8% | 1%-2% | 4%-6% | 4%-6% | 2%-4% |
| Ex 5530 | 6%-8% | 1%-2% | 4%-6% | 4%-6% | 4%-6% |
| Ex 5531 | 6%-8% | 1%-2% | 4%-6% | 4%-6% | 6%-8% |
| Ex 5532 | 6%-8% | 1%-2% | 4%-6% | 4%-6% | 8%-10% |
| Ex 5533 | 6%-8% | 1%-2% | 4%-6% | 6%-8% | 0%-1% |
| Ex 5534 | 6%-8% | 1%-2% | 4%-6% | 6%-8% | 1%-2% |
| Ex 5535 | 6%-8% | 1%-2% | 4%-6% | 6%-8% | 2%-4% |
| Ex 5536 | 6%-8% | 1%-2% | 4%-6% | 6%-8% | 4%-6% |
| Ex 5537 | 6%-8% | 1%-2% | 4%-6% | 6%-8% | 6%-8% |
| Ex 5538 | 6%-8% | 1%-2% | 4%-6% | 6%-8% | 8%-10% |
| Ex 5539 | 6%-8% | 1%-2% | 4%-6% | 8%-10% | 0%-1% |
| Ex 5540 | 6%-8% | 1%-2% | 4%-6% | 8%-10% | 1%-2% |
| Ex 5541 | 6%-8% | 1%-2% | 4%-6% | 8%-10% | 2%-4% |
| Ex 5542 | 6%-8% | 1%-2% | 4%-6% | 8%-10% | 4%-6% |
| Ex 5543 | 6%-8% | 1%-2% | 4%-6% | 8%-10% | 6%-8% |
| Ex 5544 | 6%-8% | 1%-2% | 4%-6% | 8%-10% | 8%-10% |
| Ex 5545 | 6%-8% | 1%-2% | 6%-8% | 0%-1% | 0%-1% |
| Ex 5546 | 6%-8% | 1%-2% | 6%-8% | 0%-1% | 1%-2% |
| Ex 5547 | 6%-8% | 1%-2% | 6%-8% | 0%-1% | 2%-4% |
| Ex 5548 | 6%-8% | 1%-2% | 6%-8% | 0%-1% | 4%-6% |
| Ex 5549 | 6%-8% | 1%-2% | 6%-8% | 0%-1% | 6%-8% |
| Ex 5550 | 6%-8% | 1%-2% | 6%-8% | 0%-1% | 8%-10% |
| Ex 5551 | 6%-8% | 1%-2% | 6%-8% | 1%-2% | 0%-1% |
| Ex 5552 | 6%-8% | 1%-2% | 6%-8% | 1%-2% | 1%-2% |
| Ex 5553 | 6%-8% | 1%-2% | 6%-8% | 1%-2% | 2%-4% |
| Ex 5554 | 6%-8% | 1%-2% | 6%-8% | 1%-2% | 4%-6% |
| Ex 5555 | 6%-8% | 1%-2% | 6%-8% | 1%-2% | 6%-8% |
| Ex 5556 | 6%-8% | 1%-2% | 6%-8% | 1%-2% | 8%-10% |
| Ex 5557 | 6%-8% | 1%-2% | 6%-8% | 2%-4% | 0%-1% |
| Ex 5558 | 6%-8% | 1%-2% | 6%-8% | 2%-4% | 1%-2% |
| Ex 5559 | 6%-8% | 1%-2% | 6%-8% | 2%-4% | 2%-4% |
| Ex 5560 | 6%-8% | 1%-2% | 6%-8% | 2%-4% | 4%-6% |
| Ex 5561 | 6%-8% | 1%-2% | 6%-8% | 2%-4% | 6%-8% |
| Ex 5562 | 6%-8% | 1%-2% | 6%-8% | 2%-4% | 8%-10% |
| Ex 5563 | 6%-8% | 1%-2% | 6%-8% | 4%-6% | 0%-1% |
| Ex 5564 | 6%-8% | 1%-2% | 6%-8% | 4%-6% | 1%-2% |
| Ex 5565 | 6%-8% | 1%-2% | 6%-8% | 4%-6% | 2%-4% |
| Ex 5566 | 6%-8% | 1%-2% | 6%-8% | 4%-6% | 4%-6% |
| Ex 5567 | 6%-8% | 1%-2% | 6%-8% | 4%-6% | 6%-8% |
| Ex 5568 | 6%-8% | 1%-2% | 6%-8% | 4%-6% | 8%-10% |
| Ex 5569 | 6%-8% | 1%-2% | 6%-8% | 6%-8% | 0%-1% |
| Ex 5570 | 6%-8% | 1%-2% | 6%-8% | 6%-8% | 1%-2% |
| Ex 5571 | 6%-8% | 1%-2% | 6%-8% | 6%-8% | 2%-4% |
| Ex 5572 | 6%-8% | 1%-2% | 6%-8% | 6%-8% | 4%-6% |
| Ex 5573 | 6%-8% | 1%-2% | 6%-8% | 6%-8% | 6%-8% |
| Ex 5574 | 6%-8% | 1%-2% | 6%-8% | 6%-8% | 8%-10% |
| Ex 5575 | 6%-8% | 1%-2% | 6%-8% | 8%-10% | 0%-1% |
| Ex 5576 | 6%-8% | 1%-2% | 6%-8% | 8%-10% | 1%-2% |
| Ex 5577 | 6%-8% | 1%-2% | 6%-8% | 8%-10% | 2%-4% |
| Ex 5578 | 6%-8% | 1%-2% | 6%-8% | 8%-10% | 4%-6% |
| Ex 5579 | 6%-8% | 1%-2% | 6%-8% | 8%-10% | 6%-8% |
| Ex 5580 | 6%-8% | 1%-2% | 6%-8% | 8%-10% | 8%-10% |
| Ex 5581 | 6%-8% | 1%-2% | 8%-10% | 0%-1% | 0%-1% |
| Ex 5582 | 6%-8% | 1%-2% | 8%-10% | 0%-1% | 1%-2% |
| Ex 5583 | 6%-8% | 1%-2% | 8%-10% | 0%-1% | 2%-4% |
| Ex 5584 | 6%-8% | 1%-2% | 8%-10% | 0%-1% | 4%-6% |
| Ex 5585 | 6%-8% | 1%-2% | 8%-10% | 0%-1% | 6%-8% |
| Ex 5586 | 6%-8% | 1%-2% | 8%-10% | 0%-1% | 8%-10% |
| Ex 5587 | 6%-8% | 1%-2% | 8%-10% | 1%-2% | 0%-1% |
| Ex 5588 | 6%-8% | 1%-2% | 8%-10% | 1%-2% | 1%-2% |
| Ex 5589 | 6%-8% | 1%-2% | 8%-10% | 1%-2% | 2%-4% |
| Ex 5590 | 6%-8% | 1%-2% | 8%-10% | 1%-2% | 4%-6% |
| Ex 5591 | 6%-8% | 1%-2% | 8%-10% | 1%-2% | 6%-8% |
| Ex 5592 | 6%-8% | 1%-2% | 8%-10% | 1%-2% | 8%-10% |
| Ex 5593 | 6%-8% | 1%-2% | 8%-10% | 2%-4% | 0%-1% |
| Ex 5594 | 6%-8% | 1%-2% | 8%-10% | 2%-4% | 1%-2% |
| Ex 5595 | 6%-8% | 1%-2% | 8%-10% | 2%-4% | 2%-4% |
| Ex 5596 | 6%-8% | 1%-2% | 8%-10% | 2%-4% | 4%-6% |
| Ex 5597 | 6%-8% | 1%-2% | 8%-10% | 2%-4% | 6%-8% |
| Ex 5598 | 6%-8% | 1%-2% | 8%-10% | 2%-4% | 8%-10% |
| Ex 5599 | 6%-8% | 1%-2% | 8%-10% | 4%-6% | 0%-1% |
| Ex 5600 | 6%-8% | 1%-2% | 8%-10% | 4%-6% | 1%-2% |
| Ex 5601 | 6%-8% | 1%-2% | 8%-10% | 4%-6% | 2%-4% |
| Ex 5602 | 6%-8% | 1%-2% | 8%-10% | 4%-6% | 4%-6% |
| Ex 5603 | 6%-8% | 1%-2% | 8%-10% | 4%-6% | 6%-8% |
| Ex 5604 | 6%-8% | 1%-2% | 8%-10% | 4%-6% | 8%-10% |
| Ex 5605 | 6%-8% | 1%-2% | 8%-10% | 6%-8% | 0%-1% |
| Ex 5606 | 6%-8% | 1%-2% | 8%-10% | 6%-8% | 1%-2% |
| Ex 5607 | 6%-8% | 1%-2% | 8%-10% | 6%-8% | 2%-4% |
| Ex 5608 | 6%-8% | 1%-2% | 8%-10% | 6%-8% | 4%-6% |
| Ex 5609 | 6%-8% | 1%-2% | 8%-10% | 6%-8% | 6%-8% |
| Ex 5610 | 6%-8% | 1%-2% | 8%-10% | 6%-8% | 8%-10% |
| Ex 5611 | 6%-8% | 1%-2% | 8%-10% | 8%-10% | 0%-1% |
| Ex 5612 | 6%-8% | 1%-2% | 8%-10% | 8%-10% | 1%-2% |
| Ex 5613 | 6%-8% | 1%-2% | 8%-10% | 8%-10% | 2%-4% |
| Ex 5614 | 6%-8% | 1%-2% | 8%-10% | 8%-10% | 4%-6% |
| Ex 5615 | 6%-8% | 1%-2% | 8%-10% | 8%-10% | 6%-8% |
| Ex 5616 | 6%-8% | 1%-2% | 8%-10% | 8%-10% | 8%-10% |
| Ex 5617 | 6%-8% | 2%-4% | 0%-1% | 0%-1% | 0%-1% |
| Ex 5618 | 6%-8% | 2%-4% | 0%-1% | 0%-1% | 1%-2% |
| Ex 5619 | 6%-8% | 2%-4% | 0%-1% | 0%-1% | 2%-4% |
| Ex 5620 | 6%-8% | 2%-4% | 0%-1% | 0%-1% | 4%-6% |
| Ex 5621 | 6%-8% | 2%-4% | 0%-1% | 0%-1% | 6%-8% |
| Ex 5622 | 6%-8% | 2%-4% | 0%-1% | 0%-1% | 8%-10% |
| Ex 5623 | 6%-8% | 2%-4% | 0%-1% | 1%-2% | 0%-1% |
| Ex 5624 | 6%-8% | 2%-4% | 0%-1% | 1%-2% | 1%-2% |
| Ex 5625 | 6%-8% | 2%-4% | 0%-1% | 1%-2% | 2%-4% |
| Ex 5626 | 6%-8% | 2%-4% | 0%-1% | 1%-2% | 4%-6% |
| Ex 5627 | 6%-8% | 2%-4% | 0%-1% | 1%-2% | 6%-8% |
| Ex 5628 | 6%-8% | 2%-4% | 0%-1% | 1%-2% | 8%-10% |
| Ex 5629 | 6%-8% | 2%-4% | 0%-1% | 2%-4% | 0%-1% |
| Ex 5630 | 6%-8% | 2%-4% | 0%-1% | 2%-4% | 1%-2% |
| Ex 5631 | 6%-8% | 2%-4% | 0%-1% | 2%-4% | 2%-4% |
| Ex 5632 | 6%-8% | 2%-4% | 0%-1% | 2%-4% | 4%-6% |
| Ex 5633 | 6%-8% | 2%-4% | 0%-1% | 2%-4% | 6%-8% |
| Ex 5634 | 6%-8% | 2%-4% | 0%-1% | 2%-4% | 8%-10% |
| Ex 5635 | 6%-8% | 2%-4% | 0%-1% | 4%-6% | 0%-1% |
| Ex 5636 | 6%-8% | 2%-4% | 0%-1% | 4%-6% | 1%-2% |
| Ex 5637 | 6%-8% | 2%-4% | 0%-1% | 4%-6% | 2%-4% |
| Ex 5638 | 6%-8% | 2%-4% | 0%-1% | 4%-6% | 4%-6% |
| Ex 5639 | 6%-8% | 2%-4% | 0%-1% | 4%-6% | 6%-8% |
| Ex 5640 | 6%-8% | 2%-4% | 0%-1% | 4%-6% | 8%-10% |
| Ex 5641 | 6%-8% | 2%-4% | 0%-1% | 6%-8% | 0%-1% |
| Ex 5642 | 6%-8% | 2%-4% | 0%-1% | 6%-8% | 1%-2% |
| Ex 5643 | 6%-8% | 2%-4% | 0%-1% | 6%-8% | 2%-4% |
| Ex 5644 | 6%-8% | 2%-4% | 0%-1% | 6%-8% | 4%-6% |
| Ex 5645 | 6%-8% | 2%-4% | 0%-1% | 6%-8% | 6%-8% |
| Ex 5646 | 6%-8% | 2%-4% | 0%-1% | 6%-8% | 8%-10% |
| Ex 5647 | 6%-8% | 2%-4% | 0%-1% | 8%-10% | 0%-1% |
| Ex 5648 | 6%-8% | 2%-4% | 0%-1% | 8%-10% | 1%-2% |
| Ex 5649 | 6%-8% | 2%-4% | 0%-1% | 8%-10% | 2%-4% |
| Ex 5650 | 6%-8% | 2%-4% | 0%-1% | 8%-10% | 4%-6% |
| Ex 5651 | 6%-8% | 2%-4% | 0%-1% | 8%-10% | 6%-8% |
| Ex 5652 | 6%-8% | 2%-4% | 0%-1% | 8%-10% | 8%-10% |
| Ex 5653 | 6%-8% | 2%-4% | 1%-2% | 0%-1% | 0%-1% |
| Ex 5654 | 6%-8% | 2%-4% | 1%-2% | 0%-1% | 1%-2% |
| Ex 5655 | 6%-8% | 2%-4% | 1%-2% | 0%-1% | 2%-4% |
| Ex 5656 | 6%-8% | 2%-4% | 1%-2% | 0%-1% | 4%-6% |
| Ex 5657 | 6%-8% | 2%-4% | 1%-2% | 0%-1% | 6%-8% |
| Ex 5658 | 6%-8% | 2%-4% | 1%-2% | 0%-1% | 8%-10% |
| Ex 5659 | 6%-8% | 2%-4% | 1%-2% | 1%-2% | 0%-1% |
| Ex 5660 | 6%-8% | 2%-4% | 1%-2% | 1%-2% | 1%-2% |
| Ex 5661 | 6%-8% | 2%-4% | 1%-2% | 1%-2% | 2%-4% |
| Ex 5662 | 6%-8% | 2%-4% | 1%-2% | 1%-2% | 4%-6% |
| Ex 5663 | 6%-8% | 2%-4% | 1%-2% | 1%-2% | 6%-8% |
| Ex 5664 | 6%-8% | 2%-4% | 1%-2% | 1%-2% | 8%-10% |
| Ex 5665 | 6%-8% | 2%-4% | 1%-2% | 2%-4% | 0%-1% |
| Ex 5666 | 6%-8% | 2%-4% | 1%-2% | 2%-4% | 1%-2% |
| Ex 5667 | 6%-8% | 2%-4% | 1%-2% | 2%-4% | 2%-4% |
| Ex 5668 | 6%-8% | 2%-4% | 1%-2% | 2%-4% | 4%-6% |
| Ex 5669 | 6%-8% | 2%-4% | 1%-2% | 2%-4% | 6%-8% |
| Ex 5670 | 6%-8% | 2%-4% | 1%-2% | 2%-4% | 8%-10% |
| Ex 5671 | 6%-8% | 2%-4% | 1%-2% | 4%-6% | 0%-1% |
| Ex 5672 | 6%-8% | 2%-4% | 1%-2% | 4%-6% | 1%-2% |
| Ex 5673 | 6%-8% | 2%-4% | 1%-2% | 4%-6% | 2%-4% |
| Ex 5674 | 6%-8% | 2%-4% | 1%-2% | 4%-6% | 4%-6% |
| Ex 5675 | 6%-8% | 2%-4% | 1%-2% | 4%-6% | 6%-8% |
| Ex 5676 | 6%-8% | 2%-4% | 1%-2% | 4%-6% | 8%-10% |
| Ex 5677 | 6%-8% | 2%-4% | 1%-2% | 6%-8% | 0%-1% |
| Ex 5678 | 6%-8% | 2%-4% | 1%-2% | 6%-8% | 1%-2% |
| Ex 5679 | 6%-8% | 2%-4% | 1%-2% | 6%-8% | 2%-4% |
| Ex 5680 | 6%-8% | 2%-4% | 1%-2% | 6%-8% | 4%-6% |
| Ex 5681 | 6%-8% | 2%-4% | 1%-2% | 6%-8% | 6%-8% |
| Ex 5682 | 6%-8% | 2%-4% | 1%-2% | 6%-8% | 8%-10% |
| Ex 5683 | 6%-8% | 2%-4% | 1%-2% | 8%-10% | 0%-1% |
| Ex 5684 | 6%-8% | 2%-4% | 1%-2% | 8%-10% | 1%-2% |
| Ex 5685 | 6%-8% | 2%-4% | 1%-2% | 8%-10% | 2%-4% |
| Ex 5686 | 6%-8% | 2%-4% | 1%-2% | 8%-10% | 4%-6% |
| Ex 5687 | 6%-8% | 2%-4% | 1%-2% | 8%-10% | 6%-8% |
| Ex 5688 | 6%-8% | 2%-4% | 1%-2% | 8%-10% | 8%-10% |
| Ex 5689 | 6%-8% | 2%-4% | 2%-4% | 0%-1% | 0%-1% |
| Ex 5690 | 6%-8% | 2%-4% | 2%-4% | 0%-1% | 1%-2% |
| Ex 5691 | 6%-8% | 2%-4% | 2%-4% | 0%-1% | 2%-4% |
| Ex 5692 | 6%-8% | 2%-4% | 2%-4% | 0%-1% | 4%-6% |
| Ex 5693 | 6%-8% | 2%-4% | 2%-4% | 0%-1% | 6%-8% |
| Ex 5694 | 6%-8% | 2%-4% | 2%-4% | 0%-1% | 8%-10% |
| Ex 5695 | 6%-8% | 2%-4% | 2%-4% | 1%-2% | 0%-1% |
| Ex 5696 | 6%-8% | 2%-4% | 2%-4% | 1%-2% | 1%-2% |
| Ex 5697 | 6%-8% | 2%-4% | 2%-4% | 1%-2% | 2%-4% |
| Ex 5698 | 6%-8% | 2%-4% | 2%-4% | 1%-2% | 4%-6% |
| Ex 5699 | 6%-8% | 2%-4% | 2%-4% | 1%-2% | 6%-8% |
| Ex 5700 | 6%-8% | 2%-4% | 2%-4% | 1%-2% | 8%-10% |
| Ex 5701 | 6%-8% | 2%-4% | 2%-4% | 2%-4% | 0%-1% |
| Ex 5702 | 6%-8% | 2%-4% | 2%-4% | 2%-4% | 1%-2% |
| Ex 5703 | 6%-8% | 2%-4% | 2%-4% | 2%-4% | 2%-4% |
| Ex 5704 | 6%-8% | 2%-4% | 2%-4% | 2%-4% | 4%-6% |
| Ex 5705 | 6%-8% | 2%-4% | 2%-4% | 2%-4% | 6%-8% |
| Ex 5706 | 6%-8% | 2%-4% | 2%-4% | 2%-4% | 8%-10% |
| Ex 5707 | 6%-8% | 2%-4% | 2%-4% | 4%-6% | 0%-1% |
| Ex 5708 | 6%-8% | 2%-4% | 2%-4% | 4%-6% | 1%-2% |
| Ex 5709 | 6%-8% | 2%-4% | 2%-4% | 4%-6% | 2%-4% |
| Ex 5710 | 6%-8% | 2%-4% | 2%-4% | 4%-6% | 4%-6% |
| Ex 5711 | 6%-8% | 2%-4% | 2%-4% | 4%-6% | 6%-8% |
| Ex 5712 | 6%-8% | 2%-4% | 2%-4% | 4%-6% | 8%-10% |
| Ex 5713 | 6%-8% | 2%-4% | 2%-4% | 6%-8% | 0%-1% |
| Ex 5714 | 6%-8% | 2%-4% | 2%-4% | 6%-8% | 1%-2% |
| Ex 5715 | 6%-8% | 2%-4% | 2%-4% | 6%-8% | 2%-4% |
| Ex 5716 | 6%-8% | 2%-4% | 2%-4% | 6%-8% | 4%-6% |
| Ex 5717 | 6%-8% | 2%-4% | 2%-4% | 6%-8% | 6%-8% |
| Ex 5718 | 6%-8% | 2%-4% | 2%-4% | 6%-8% | 8%-10% |
| Ex 5719 | 6%-8% | 2%-4% | 2%-4% | 8%-10% | 0%-1% |
| Ex 5720 | 6%-8% | 2%-4% | 2%-4% | 8%-10% | 1%-2% |
| Ex 5721 | 6%-8% | 2%-4% | 2%-4% | 8%-10% | 2%-4% |
| Ex 5722 | 6%-8% | 2%-4% | 2%-4% | 8%-10% | 4%-6% |
| Ex 5723 | 6%-8% | 2%-4% | 2%-4% | 8%-10% | 6%-8% |
| Ex 5724 | 6%-8% | 2%-4% | 2%-4% | 8%-10% | 8%-10% |
| Ex 5725 | 6%-8% | 2%-4% | 4%-6% | 0%-1% | 0%-1% |
| Ex 5726 | 6%-8% | 2%-4% | 4%-6% | 0%-1% | 1%-2% |
| Ex 5727 | 6%-8% | 2%-4% | 4%-6% | 0%-1% | 2%-4% |
| Ex 5728 | 6%-8% | 2%-4% | 4%-6% | 0%-1% | 4%-6% |
| Ex 5729 | 6%-8% | 2%-4% | 4%-6% | 0%-1% | 6%-8% |
| Ex 5730 | 6%-8% | 2%-4% | 4%-6% | 0%-1% | 8%-10% |
| Ex 5731 | 6%-8% | 2%-4% | 4%-6% | 1%-2% | 0%-1% |
| Ex 5732 | 6%-8% | 2%-4% | 4%-6% | 1%-2% | 1%-2% |
| Ex 5733 | 6%-8% | 2%-4% | 4%-6% | 1%-2% | 2%-4% |
| Ex 5734 | 6%-8% | 2%-4% | 4%-6% | 1%-2% | 4%-6% |
| Ex 5735 | 6%-8% | 2%-4% | 4%-6% | 1%-2% | 6%-8% |
| Ex 5736 | 6%-8% | 2%-4% | 4%-6% | 1%-2% | 8%-10% |
| Ex 5737 | 6%-8% | 2%-4% | 4%-6% | 2%-4% | 0%-1% |
| Ex 5738 | 6%-8% | 2%-4% | 4%-6% | 2%-4% | 1%-2% |
| Ex 5739 | 6%-8% | 2%-4% | 4%-6% | 2%-4% | 2%-4% |
| Ex 5740 | 6%-8% | 2%-4% | 4%-6% | 2%-4% | 4%-6% |
| Ex 5741 | 6%-8% | 2%-4% | 4%-6% | 2%-4% | 6%-8% |
| Ex 5742 | 6%-8% | 2%-4% | 4%-6% | 2%-4% | 8%-10% |
| Ex 5743 | 6%-8% | 2%-4% | 4%-6% | 4%-6% | 0%-1% |
| Ex 5744 | 6%-8% | 2%-4% | 4%-6% | 4%-6% | 1%-2% |
| Ex 5745 | 6%-8% | 2%-4% | 4%-6% | 4%-6% | 2%-4% |
| Ex 5746 | 6%-8% | 2%-4% | 4%-6% | 4%-6% | 4%-6% |
| Ex 5747 | 6%-8% | 2%-4% | 4%-6% | 4%-6% | 6%-8% |
| Ex 5748 | 6%-8% | 2%-4% | 4%-6% | 4%-6% | 8%-10% |
| Ex 5749 | 6%-8% | 2%-4% | 4%-6% | 6%-8% | 0%-1% |
| Ex 5750 | 6%-8% | 2%-4% | 4%-6% | 6%-8% | 1%-2% |
| Ex 5751 | 6%-8% | 2%-4% | 4%-6% | 6%-8% | 2%-4% |
| Ex 5752 | 6%-8% | 2%-4% | 4%-6% | 6%-8% | 4%-6% |
| Ex 5753 | 6%-8% | 2%-4% | 4%-6% | 6%-8% | 6%-8% |
| Ex 5754 | 6%-8% | 2%-4% | 4%-6% | 6%-8% | 8%-10% |
| Ex 5755 | 6%-8% | 2%-4% | 4%-6% | 8%-10% | 0%-1% |
| Ex 5756 | 6%-8% | 2%-4% | 4%-6% | 8%-10% | 1%-2% |
| Ex 5757 | 6%-8% | 2%-4% | 4%-6% | 8%-10% | 2%-4% |
| Ex 5758 | 6%-8% | 2%-4% | 4%-6% | 8%-10% | 4%-6% |
| Ex 5759 | 6%-8% | 2%-4% | 4%-6% | 8%-10% | 6%-8% |
| Ex 5760 | 6%-8% | 2%-4% | 4%-6% | 8%-10% | 8%-10% |
| Ex 5761 | 6%-8% | 2%-4% | 6%-8% | 0%-1% | 0%-1% |
| Ex 5762 | 6%-8% | 2%-4% | 6%-8% | 0%-1% | 1%-2% |
| Ex 5763 | 6%-8% | 2%-4% | 6%-8% | 0%-1% | 2%-4% |
| Ex 5764 | 6%-8% | 2%-4% | 6%-8% | 0%-1% | 4%-6% |
| Ex 5765 | 6%-8% | 2%-4% | 6%-8% | 0%-1% | 6%-8% |
| Ex 5766 | 6%-8% | 2%-4% | 6%-8% | 0%-1% | 8%-10% |
| Ex 5767 | 6%-8% | 2%-4% | 6%-8% | 1%-2% | 0%-1% |
| Ex 5768 | 6%-8% | 2%-4% | 6%-8% | 1%-2% | 1%-2% |
| Ex 5769 | 6%-8% | 2%-4% | 6%-8% | 1%-2% | 2%-4% |
| Ex 5770 | 6%-8% | 2%-4% | 6%-8% | 1%-2% | 4%-6% |
| Ex 5771 | 6%-8% | 2%-4% | 6%-8% | 1%-2% | 6%-8% |
| Ex 5772 | 6%-8% | 2%-4% | 6%-8% | 1%-2% | 8%-10% |
| Ex 5773 | 6%-8% | 2%-4% | 6%-8% | 2%-4% | 0%-1% |
| Ex 5774 | 6%-8% | 2%-4% | 6%-8% | 2%-4% | 1%-2% |
| Ex 5775 | 6%-8% | 2%-4% | 6%-8% | 2%-4% | 2%-4% |
| Ex 5776 | 6%-8% | 2%-4% | 6%-8% | 2%-4% | 4%-6% |
| Ex 5777 | 6%-8% | 2%-4% | 6%-8% | 2%-4% | 6%-8% |
| Ex 5778 | 6%-8% | 2%-4% | 6%-8% | 2%-4% | 8%-10% |
| Ex 5779 | 6%-8% | 2%-4% | 6%-8% | 4%-6% | 0%-1% |
| Ex 5780 | 6%-8% | 2%-4% | 6%-8% | 4%-6% | 1%-2% |
| Ex 5781 | 6%-8% | 2%-4% | 6%-8% | 4%-6% | 2%-4% |
| Ex 5782 | 6%-8% | 2%-4% | 6%-8% | 4%-6% | 4%-6% |
| Ex 5783 | 6%-8% | 2%-4% | 6%-8% | 4%-6% | 6%-8% |
| Ex 5784 | 6%-8% | 2%-4% | 6%-8% | 4%-6% | 8%-10% |
| Ex 5785 | 6%-8% | 2%-4% | 6%-8% | 6%-8% | 0%-1% |
| Ex 5786 | 6%-8% | 2%-4% | 6%-8% | 6%-8% | 1%-2% |
| Ex 5787 | 6%-8% | 2%-4% | 6%-8% | 6%-8% | 2%-4% |
| Ex 5788 | 6%-8% | 2%-4% | 6%-8% | 6%-8% | 4%-6% |
| Ex 5789 | 6%-8% | 2%-4% | 6%-8% | 6%-8% | 6%-8% |
| Ex 5790 | 6%-8% | 2%-4% | 6%-8% | 6%-8% | 8%-10% |
| Ex 5791 | 6%-8% | 2%-4% | 6%-8% | 8%-10% | 0%-1% |
| Ex 5792 | 6%-8% | 2%-4% | 6%-8% | 8%-10% | 1%-2% |
| Ex 5793 | 6%-8% | 2%-4% | 6%-8% | 8%-10% | 2%-4% |
| Ex 5794 | 6%-8% | 2%-4% | 6%-8% | 8%-10% | 4%-6% |
| Ex 5795 | 6%-8% | 2%-4% | 6%-8% | 8%-10% | 6%-8% |
| Ex 5796 | 6%-8% | 2%-4% | 6%-8% | 8%-10% | 8%-10% |
| Ex 5797 | 6%-8% | 2%-4% | 8%-10% | 0%-1% | 0%-1% |
| Ex 5798 | 6%-8% | 2%-4% | 8%-10% | 0%-1% | 1%-2% |
| Ex 5799 | 6%-8% | 2%-4% | 8%-10% | 0%-1% | 2%-4% |
| Ex 5800 | 6%-8% | 2%-4% | 8%-10% | 0%-1% | 4%-6% |
| Ex 5801 | 6%-8% | 2%-4% | 8%-10% | 0%-1% | 6%-8% |
| Ex 5802 | 6%-8% | 2%-4% | 8%-10% | 0%-1% | 8%-10% |
| Ex 5803 | 6%-8% | 2%-4% | 8%-10% | 1%-2% | 0%-1% |
| Ex 5804 | 6%-8% | 2%-4% | 8%-10% | 1%-2% | 1%-2% |
| Ex 5805 | 6%-8% | 2%-4% | 8%-10% | 1%-2% | 2%-4% |
| Ex 5806 | 6%-8% | 2%-4% | 8%-10% | 1%-2% | 4%-6% |
| Ex 5807 | 6%-8% | 2%-4% | 8%-10% | 1%-2% | 6%-8% |
| Ex 5808 | 6%-8% | 2%-4% | 8%-10% | 1%-2% | 8%-10% |
| Ex 5809 | 6%-8% | 2%-4% | 8%-10% | 2%-4% | 0%-1% |
| Ex 5810 | 6%-8% | 2%-4% | 8%-10% | 2%-4% | 1%-2% |
| Ex 5811 | 6%-8% | 2%-4% | 8%-10% | 2%-4% | 2%-4% |
| Ex 5812 | 6%-8% | 2%-4% | 8%-10% | 2%-4% | 4%-6% |
| Ex 5813 | 6%-8% | 2%-4% | 8%-10% | 2%-4% | 6%-8% |
| Ex 5814 | 6%-8% | 2%-4% | 8%-10% | 2%-4% | 8%-10% |
| Ex 5815 | 6%-8% | 2%-4% | 8%-10% | 4%-6% | 0%-1% |
| Ex 5816 | 6%-8% | 2%-4% | 8%-10% | 4%-6% | 1%-2% |
| Ex 5817 | 6%-8% | 2%-4% | 8%-10% | 4%-6% | 2%-4% |
| Ex 5818 | 6%-8% | 2%-4% | 8%-10% | 4%-6% | 4%-6% |
| Ex 5819 | 6%-8% | 2%-4% | 8%-10% | 4%-6% | 6%-8% |
| Ex 5820 | 6%-8% | 2%-4% | 8%-10% | 4%-6% | 8%-10% |
| Ex 5821 | 6%-8% | 2%-4% | 8%-10% | 6%-8% | 0%-1% |
| Ex 5822 | 6%-8% | 2%-4% | 8%-10% | 6%-8% | 1%-2% |
| Ex 5823 | 6%-8% | 2%-4% | 8%-10% | 6%-8% | 2%-4% |
| Ex 5824 | 6%-8% | 2%-4% | 8%-10% | 6%-8% | 4%-6% |
| Ex 5825 | 6%-8% | 2%-4% | 8%-10% | 6%-8% | 6%-8% |
| Ex 5826 | 6%-8% | 2%-4% | 8%-10% | 6%-8% | 8%-10% |
| Ex 5827 | 6%-8% | 2%-4% | 8%-10% | 8%-10% | 0%-1% |
| Ex 5828 | 6%-8% | 2%-4% | 8%-10% | 8%-10% | 1%-2% |
| Ex 5829 | 6%-8% | 2%-4% | 8%-10% | 8%-10% | 2%-4% |
| Ex 5830 | 6%-8% | 2%-4% | 8%-10% | 8%-10% | 4%-6% |
| Ex 5831 | 6%-8% | 2%-4% | 8%-10% | 8%-10% | 6%-8% |
| Ex 5832 | 6%-8% | 2%-4% | 8%-10% | 8%-10% | 8%-10% |
| Ex 5833 | 6%-8% | 4%-6% | 0%-1% | 0%-1% | 0%-1% |
| Ex 5834 | 6%-8% | 4%-6% | 0%-1% | 0%-1% | 1%-2% |
| Ex 5835 | 6%-8% | 4%-6% | 0%-1% | 0%-1% | 2%-4% |
| Ex 5836 | 6%-8% | 4%-6% | 0%-1% | 0%-1% | 4%-6% |
| Ex 5837 | 6%-8% | 4%-6% | 0%-1% | 0%-1% | 6%-8% |
| Ex 5838 | 6%-8% | 4%-6% | 0%-1% | 0%-1% | 8%-10% |
| Ex 5839 | 6%-8% | 4%-6% | 0%-1% | 1%-2% | 0%-1% |
| Ex 5840 | 6%-8% | 4%-6% | 0%-1% | 1%-2% | 1%-2% |
| Ex 5841 | 6%-8% | 4%-6% | 0%-1% | 1%-2% | 2%-4% |
| Ex 5842 | 6%-8% | 4%-6% | 0%-1% | 1%-2% | 4%-6% |
| Ex 5843 | 6%-8% | 4%-6% | 0%-1% | 1%-2% | 6%-8% |
| Ex 5844 | 6%-8% | 4%-6% | 0%-1% | 1%-2% | 8%-10% |
| Ex 5845 | 6%-8% | 4%-6% | 0%-1% | 2%-4% | 0%-1% |
| Ex 5846 | 6%-8% | 4%-6% | 0%-1% | 2%-4% | 1%-2% |
| Ex 5847 | 6%-8% | 4%-6% | 0%-1% | 2%-4% | 2%-4% |
| Ex 5848 | 6%-8% | 4%-6% | 0%-1% | 2%-4% | 4%-6% |
| Ex 5849 | 6%-8% | 4%-6% | 0%-1% | 2%-4% | 6%-8% |
| Ex 5850 | 6%-8% | 4%-6% | 0%-1% | 2%-4% | 8%-10% |
| Ex 5851 | 6%-8% | 4%-6% | 0%-1% | 4%-6% | 0%-1% |
| Ex 5852 | 6%-8% | 4%-6% | 0%-1% | 4%-6% | 1%-2% |
| Ex 5853 | 6%-8% | 4%-6% | 0%-1% | 4%-6% | 2%-4% |
| Ex 5854 | 6%-8% | 4%-6% | 0%-1% | 4%-6% | 4%-6% |
| Ex 5855 | 6%-8% | 4%-6% | 0%-1% | 4%-6% | 6%-8% |
| Ex 5856 | 6%-8% | 4%-6% | 0%-1% | 4%-6% | 8%-10% |
| Ex 5857 | 6%-8% | 4%-6% | 0%-1% | 6%-8% | 0%-1% |
| Ex 5858 | 6%-8% | 4%-6% | 0%-1% | 6%-8% | 1%-2% |
| Ex 5859 | 6%-8% | 4%-6% | 0%-1% | 6%-8% | 2%-4% |
| Ex 5860 | 6%-8% | 4%-6% | 0%-1% | 6%-8% | 4%-6% |
| Ex 5861 | 6%-8% | 4%-6% | 0%-1% | 6%-8% | 6%-8% |
| Ex 5862 | 6%-8% | 4%-6% | 0%-1% | 6%-8% | 8%-10% |
| Ex 5863 | 6%-8% | 4%-6% | 0%-1% | 8%-10% | 0%-1% |
| Ex 5864 | 6%-8% | 4%-6% | 0%-1% | 8%-10% | 1%-2% |
| Ex 5865 | 6%-8% | 4%-6% | 0%-1% | 8%-10% | 2%-4% |
| Ex 5866 | 6%-8% | 4%-6% | 0%-1% | 8%-10% | 4%-6% |
| Ex 5867 | 6%-8% | 4%-6% | 0%-1% | 8%-10% | 6%-8% |
| Ex 5868 | 6%-8% | 4%-6% | 0%-1% | 8%-10% | 8%-10% |
| Ex 5869 | 6%-8% | 4%-6% | 1%-2% | 0%-1% | 0%-1% |
| Ex 5870 | 6%-8% | 4%-6% | 1%-2% | 0%-1% | 1%-2% |
| Ex 5871 | 6%-8% | 4%-6% | 1%-2% | 0%-1% | 2%-4% |
| Ex 5872 | 6%-8% | 4%-6% | 1%-2% | 0%-1% | 4%-6% |
| Ex 5873 | 6%-8% | 4%-6% | 1%-2% | 0%-1% | 6%-8% |
| Ex 5874 | 6%-8% | 4%-6% | 1%-2% | 0%-1% | 8%-10% |
| Ex 5875 | 6%-8% | 4%-6% | 1%-2% | 1%-2% | 0%-1% |
| Ex 5876 | 6%-8% | 4%-6% | 1%-2% | 1%-2% | 1%-2% |
| Ex 5877 | 6%-8% | 4%-6% | 1%-2% | 1%-2% | 2%-4% |
| Ex 5878 | 6%-8% | 4%-6% | 1%-2% | 1%-2% | 4%-6% |
| Ex 5879 | 6%-8% | 4%-6% | 1%-2% | 1%-2% | 6%-8% |
| Ex 5880 | 6%-8% | 4%-6% | 1%-2% | 1%-2% | 8%-10% |
| Ex 5881 | 6%-8% | 4%-6% | 1%-2% | 2%-4% | 0%-1% |
| Ex 5882 | 6%-8% | 4%-6% | 1%-2% | 2%-4% | 1%-2% |
| Ex 5883 | 6%-8% | 4%-6% | 1%-2% | 2%-4% | 2%-4% |
| Ex 5884 | 6%-8% | 4%-6% | 1%-2% | 2%-4% | 4%-6% |
| Ex 5885 | 6%-8% | 4%-6% | 1%-2% | 2%-4% | 6%-8% |
| Ex 5886 | 6%-8% | 4%-6% | 1%-2% | 2%-4% | 8%-10% |
| Ex 5887 | 6%-8% | 4%-6% | 1%-2% | 4%-6% | 0%-1% |
| Ex 5888 | 6%-8% | 4%-6% | 1%-2% | 4%-6% | 1%-2% |
| Ex 5889 | 6%-8% | 4%-6% | 1%-2% | 4%-6% | 2%-4% |
| Ex 5890 | 6%-8% | 4%-6% | 1%-2% | 4%-6% | 4%-6% |
| Ex 5891 | 6%-8% | 4%-6% | 1%-2% | 4%-6% | 6%-8% |
| Ex 5892 | 6%-8% | 4%-6% | 1%-2% | 4%-6% | 8%-10% |
| Ex 5893 | 6%-8% | 4%-6% | 1%-2% | 6%-8% | 0%-1% |
| Ex 5894 | 6%-8% | 4%-6% | 1%-2% | 6%-8% | 1%-2% |
| Ex 5895 | 6%-8% | 4%-6% | 1%-2% | 6%-8% | 2%-4% |
| Ex 5896 | 6%-8% | 4%-6% | 1%-2% | 6%-8% | 4%-6% |
| Ex 5897 | 6%-8% | 4%-6% | 1%-2% | 6%-8% | 6%-8% |
| Ex 5898 | 6%-8% | 4%-6% | 1%-2% | 6%-8% | 8%-10% |
| Ex 5899 | 6%-8% | 4%-6% | 1%-2% | 8%-10% | 0%-1% |
| Ex 5900 | 6%-8% | 4%-6% | 1%-2% | 8%-10% | 1%-2% |
| Ex 5901 | 6%-8% | 4%-6% | 1%-2% | 8%-10% | 2%-4% |
| Ex 5902 | 6%-8% | 4%-6% | 1%-2% | 8%-10% | 4%-6% |
| Ex 5903 | 6%-8% | 4%-6% | 1%-2% | 8%-10% | 6%-8% |
| Ex 5904 | 6%-8% | 4%-6% | 1%-2% | 8%-10% | 8%-10% |
| Ex 5905 | 6%-8% | 4%-6% | 2%-4% | 0%-1% | 0%-1% |
| Ex 5906 | 6%-8% | 4%-6% | 2%-4% | 0%-1% | 1%-2% |
| Ex 5907 | 6%-8% | 4%-6% | 2%-4% | 0%-1% | 2%-4% |
| Ex 5908 | 6%-8% | 4%-6% | 2%-4% | 0%-1% | 4%-6% |
| Ex 5909 | 6%-8% | 4%-6% | 2%-4% | 0%-1% | 6%-8% |
| Ex 5910 | 6%-8% | 4%-6% | 2%-4% | 0%-1% | 8%-10% |
| Ex 5911 | 6%-8% | 4%-6% | 2%-4% | 1%-2% | 0%-1% |
| Ex 5912 | 6%-8% | 4%-6% | 2%-4% | 1%-2% | 1%-2% |
| Ex 5913 | 6%-8% | 4%-6% | 2%-4% | 1%-2% | 2%-4% |
| Ex 5914 | 6%-8% | 4%-6% | 2%-4% | 1%-2% | 4%-6% |
| Ex 5915 | 6%-8% | 4%-6% | 2%-4% | 1%-2% | 6%-8% |
| Ex 5916 | 6%-8% | 4%-6% | 2%-4% | 1%-2% | 8%-10% |
| Ex 5917 | 6%-8% | 4%-6% | 2%-4% | 2%-4% | 0%-1% |
| Ex 5918 | 6%-8% | 4%-6% | 2%-4% | 2%-4% | 1%-2% |
| Ex 5919 | 6%-8% | 4%-6% | 2%-4% | 2%-4% | 2%-4% |
| Ex 5920 | 6%-8% | 4%-6% | 2%-4% | 2%-4% | 4%-6% |
| Ex 5921 | 6%-8% | 4%-6% | 2%-4% | 2%-4% | 6%-8% |
| Ex 5922 | 6%-8% | 4%-6% | 2%-4% | 2%-4% | 8%-10% |
| Ex 5923 | 6%-8% | 4%-6% | 2%-4% | 4%-6% | 0%-1% |
| Ex 5924 | 6%-8% | 4%-6% | 2%-4% | 4%-6% | 1%-2% |
| Ex 5925 | 6%-8% | 4%-6% | 2%-4% | 4%-6% | 2%-4% |
| Ex 5926 | 6%-8% | 4%-6% | 2%-4% | 4%-6% | 4%-6% |
| Ex 5927 | 6%-8% | 4%-6% | 2%-4% | 4%-6% | 6%-8% |
| Ex 5928 | 6%-8% | 4%-6% | 2%-4% | 4%-6% | 8%-10% |
| Ex 5929 | 6%-8% | 4%-6% | 2%-4% | 6%-8% | 0%-1% |
| Ex 5930 | 6%-8% | 4%-6% | 2%-4% | 6%-8% | 1%-2% |
| Ex 5931 | 6%-8% | 4%-6% | 2%-4% | 6%-8% | 2%-4% |
| Ex 5932 | 6%-8% | 4%-6% | 2%-4% | 6%-8% | 4%-6% |
| Ex 5933 | 6%-8% | 4%-6% | 2%-4% | 6%-8% | 6%-8% |
| Ex 5934 | 6%-8% | 4%-6% | 2%-4% | 6%-8% | 8%-10% |
| Ex 5935 | 6%-8% | 4%-6% | 2%-4% | 8%-10% | 0%-1% |
| Ex 5936 | 6%-8% | 4%-6% | 2%-4% | 8%-10% | 1%-2% |
| Ex 5937 | 6%-8% | 4%-6% | 2%-4% | 8%-10% | 2%-4% |
| Ex 5938 | 6%-8% | 4%-6% | 2%-4% | 8%-10% | 4%-6% |
| Ex 5939 | 6%-8% | 4%-6% | 2%-4% | 8%-10% | 6%-8% |
| Ex 5940 | 6%-8% | 4%-6% | 2%-4% | 8%-10% | 8%-10% |
| Ex 5941 | 6%-8% | 4%-6% | 4%-6% | 0%-1% | 0%-1% |
| Ex 5942 | 6%-8% | 4%-6% | 4%-6% | 0%-1% | 1%-2% |
| Ex 5943 | 6%-8% | 4%-6% | 4%-6% | 0%-1% | 2%-4% |
| Ex 5944 | 6%-8% | 4%-6% | 4%-6% | 0%-1% | 4%-6% |
| Ex 5945 | 6%-8% | 4%-6% | 4%-6% | 0%-1% | 6%-8% |
| Ex 5946 | 6%-8% | 4%-6% | 4%-6% | 0%-1% | 8%-10% |
| Ex 5947 | 6%-8% | 4%-6% | 4%-6% | 1%-2% | 0%-1% |
| Ex 5948 | 6%-8% | 4%-6% | 4%-6% | 1%-2% | 1%-2% |
| Ex 5949 | 6%-8% | 4%-6% | 4%-6% | 1%-2% | 2%-4% |
| Ex 5950 | 6%-8% | 4%-6% | 4%-6% | 1%-2% | 4%-6% |
| Ex 5951 | 6%-8% | 4%-6% | 4%-6% | 1%-2% | 6%-8% |
| Ex 5952 | 6%-8% | 4%-6% | 4%-6% | 1%-2% | 8%-10% |
| Ex 5953 | 6%-8% | 4%-6% | 4%-6% | 2%-4% | 0%-1% |
| Ex 5954 | 6%-8% | 4%-6% | 4%-6% | 2%-4% | 1%-2% |
| Ex 5955 | 6%-8% | 4%-6% | 4%-6% | 2%-4% | 2%-4% |
| Ex 5956 | 6%-8% | 4%-6% | 4%-6% | 2%-4% | 4%-6% |
| Ex 5957 | 6%-8% | 4%-6% | 4%-6% | 2%-4% | 6%-8% |
| Ex 5958 | 6%-8% | 4%-6% | 4%-6% | 2%-4% | 8%-10% |
| Ex 5959 | 6%-8% | 4%-6% | 4%-6% | 4%-6% | 0%-1% |
| Ex 5960 | 6%-8% | 4%-6% | 4%-6% | 4%-6% | 1%-2% |
| Ex 5961 | 6%-8% | 4%-6% | 4%-6% | 4%-6% | 2%-4% |
| Ex 5962 | 6%-8% | 4%-6% | 4%-6% | 4%-6% | 4%-6% |
| Ex 5963 | 6%-8% | 4%-6% | 4%-6% | 4%-6% | 6%-8% |
| Ex 5964 | 6%-8% | 4%-6% | 4%-6% | 4%-6% | 8%-10% |
| Ex 5965 | 6%-8% | 4%-6% | 4%-6% | 6%-8% | 0%-1% |
| Ex 5966 | 6%-8% | 4%-6% | 4%-6% | 6%-8% | 1%-2% |
| Ex 5967 | 6%-8% | 4%-6% | 4%-6% | 6%-8% | 2%-4% |
| Ex 5968 | 6%-8% | 4%-6% | 4%-6% | 6%-8% | 4%-6% |
| Ex 5969 | 6%-8% | 4%-6% | 4%-6% | 6%-8% | 6%-8% |
| Ex 5970 | 6%-8% | 4%-6% | 4%-6% | 6%-8% | 8%-10% |
| Ex 5971 | 6%-8% | 4%-6% | 4%-6% | 8%-10% | 0%-1% |
| Ex 5972 | 6%-8% | 4%-6% | 4%-6% | 8%-10% | 1%-2% |
| Ex 5973 | 6%-8% | 4%-6% | 4%-6% | 8%-10% | 2%-4% |
| Ex 5974 | 6%-8% | 4%-6% | 4%-6% | 8%-10% | 4%-6% |
| Ex 5975 | 6%-8% | 4%-6% | 4%-6% | 8%-10% | 6%-8% |
| Ex 5976 | 6%-8% | 4%-6% | 4%-6% | 8%-10% | 8%-10% |
| Ex 5977 | 6%-8% | 4%-6% | 6%-8% | 0%-1% | 0%-1% |
| Ex 5978 | 6%-8% | 4%-6% | 6%-8% | 0%-1% | 1%-2% |
| Ex 5979 | 6%-8% | 4%-6% | 6%-8% | 0%-1% | 2%-4% |
| Ex 5980 | 6%-8% | 4%-6% | 6%-8% | 0%-1% | 4%-6% |
| Ex 5981 | 6%-8% | 4%-6% | 6%-8% | 0%-1% | 6%-8% |
| Ex 5982 | 6%-8% | 4%-6% | 6%-8% | 0%-1% | 8%-10% |
| Ex 5983 | 6%-8% | 4%-6% | 6%-8% | 1%-2% | 0%-1% |
| Ex 5984 | 6%-8% | 4%-6% | 6%-8% | 1%-2% | 1%-2% |
| Ex 5985 | 6%-8% | 4%-6% | 6%-8% | 1%-2% | 2%-4% |
| Ex 5986 | 6%-8% | 4%-6% | 6%-8% | 1%-2% | 4%-6% |
| Ex 5987 | 6%-8% | 4%-6% | 6%-8% | 1%-2% | 6%-8% |
| Ex 5988 | 6%-8% | 4%-6% | 6%-8% | 1%-2% | 8%-10% |
| Ex 5989 | 6%-8% | 4%-6% | 6%-8% | 2%-4% | 0%-1% |
| Ex 5990 | 6%-8% | 4%-6% | 6%-8% | 2%-4% | 1%-2% |
| Ex 5991 | 6%-8% | 4%-6% | 6%-8% | 2%-4% | 2%-4% |
| Ex 5992 | 6%-8% | 4%-6% | 6%-8% | 2%-4% | 4%-6% |
| Ex 5993 | 6%-8% | 4%-6% | 6%-8% | 2%-4% | 6%-8% |
| Ex 5994 | 6%-8% | 4%-6% | 6%-8% | 2%-4% | 8%-10% |
| Ex 5995 | 6%-8% | 4%-6% | 6%-8% | 4%-6% | 0%-1% |
| Ex 5996 | 6%-8% | 4%-6% | 6%-8% | 4%-6% | 1%-2% |
| Ex 5997 | 6%-8% | 4%-6% | 6%-8% | 4%-6% | 2%-4% |
| Ex 5998 | 6%-8% | 4%-6% | 6%-8% | 4%-6% | 4%-6% |
| Ex 5999 | 6%-8% | 4%-6% | 6%-8% | 4%-6% | 6%-8% |
| Ex 6000 | 6%-8% | 4%-6% | 6%-8% | 4%-6% | 8%-10% |
| Ex 6001 | 6%-8% | 4%-6% | 6%-8% | 6%-8% | 0%-1% |
| Ex 6002 | 6%-8% | 4%-6% | 6%-8% | 6%-8% | 1%-2% |
| Ex 6003 | 6%-8% | 4%-6% | 6%-8% | 6%-8% | 2%-4% |
| Ex 6004 | 6%-8% | 4%-6% | 6%-8% | 6%-8% | 4%-6% |
| Ex 6005 | 6%-8% | 4%-6% | 6%-8% | 6%-8% | 6%-8% |
| Ex 6006 | 6%-8% | 4%-6% | 6%-8% | 6%-8% | 8%-10% |
| Ex 6007 | 6%-8% | 4%-6% | 6%-8% | 8%-10% | 0%-1% |
| Ex 6008 | 6%-8% | 4%-6% | 6%-8% | 8%-10% | 1%-2% |
| Ex 6009 | 6%-8% | 4%-6% | 6%-8% | 8%-10% | 2%-4% |
| Ex 6010 | 6%-8% | 4%-6% | 6%-8% | 8%-10% | 4%-6% |
| Ex 6011 | 6%-8% | 4%-6% | 6%-8% | 8%-10% | 6%-8% |
| Ex 6012 | 6%-8% | 4%-6% | 6%-8% | 8%-10% | 8%-10% |
| Ex 6013 | 6%-8% | 4%-6% | 8%-10% | 0%-1% | 0%-1% |
| Ex 6014 | 6%-8% | 4%-6% | 8%-10% | 0%-1% | 1%-2% |
| Ex 6015 | 6%-8% | 4%-6% | 8%-10% | 0%-1% | 2%-4% |
| Ex 6016 | 6%-8% | 4%-6% | 8%-10% | 0%-1% | 4%-6% |
| Ex 6017 | 6%-8% | 4%-6% | 8%-10% | 0%-1% | 6%-8% |
| Ex 6018 | 6%-8% | 4%-6% | 8%-10% | 0%-1% | 8%-10% |
| Ex 6019 | 6%-8% | 4%-6% | 8%-10% | 1%-2% | 0%-1% |
| Ex 6020 | 6%-8% | 4%-6% | 8%-10% | 1%-2% | 1%-2% |
| Ex 6021 | 6%-8% | 4%-6% | 8%-10% | 1%-2% | 2%-4% |
| Ex 6022 | 6%-8% | 4%-6% | 8%-10% | 1%-2% | 4%-6% |
| Ex 6023 | 6%-8% | 4%-6% | 8%-10% | 1%-2% | 6%-8% |
| Ex 6024 | 6%-8% | 4%-6% | 8%-10% | 1%-2% | 8%-10% |
| Ex 6025 | 6%-8% | 4%-6% | 8%-10% | 2%-4% | 0%-1% |
| Ex 6026 | 6%-8% | 4%-6% | 8%-10% | 2%-4% | 1%-2% |
| Ex 6027 | 6%-8% | 4%-6% | 8%-10% | 2%-4% | 2%-4% |
| Ex 6028 | 6%-8% | 4%-6% | 8%-10% | 2%-4% | 4%-6% |
| Ex 6029 | 6%-8% | 4%-6% | 8%-10% | 2%-4% | 6%-8% |
| Ex 6030 | 6%-8% | 4%-6% | 8%-10% | 2%-4% | 8%-10% |
| Ex 6031 | 6%-8% | 4%-6% | 8%-10% | 4%-6% | 0%-1% |
| Ex 6032 | 6%-8% | 4%-6% | 8%-10% | 4%-6% | 1%-2% |
| Ex 6033 | 6%-8% | 4%-6% | 8%-10% | 4%-6% | 2%-4% |
| Ex 6034 | 6%-8% | 4%-6% | 8%-10% | 4%-6% | 4%-6% |
| Ex 6035 | 6%-8% | 4%-6% | 8%-10% | 4%-6% | 6%-8% |
| Ex 6036 | 6%-8% | 4%-6% | 8%-10% | 4%-6% | 8%-10% |
| Ex 6037 | 6%-8% | 4%-6% | 8%-10% | 6%-8% | 0%-1% |
| Ex 6038 | 6%-8% | 4%-6% | 8%-10% | 6%-8% | 1%-2% |
| Ex 6039 | 6%-8% | 4%-6% | 8%-10% | 6%-8% | 2%-4% |
| Ex 6040 | 6%-8% | 4%-6% | 8%-10% | 6%-8% | 4%-6% |
| Ex 6041 | 6%-8% | 4%-6% | 8%-10% | 6%-8% | 6%-8% |
| Ex 6042 | 6%-8% | 4%-6% | 8%-10% | 6%-8% | 8%-10% |
| Ex 6043 | 6%-8% | 4%-6% | 8%-10% | 8%-10% | 0%-1% |
| Ex 6044 | 6%-8% | 4%-6% | 8%-10% | 8%-10% | 1%-2% |
| Ex 6045 | 6%-8% | 4%-6% | 8%-10% | 8%-10% | 2%-4% |
| Ex 6046 | 6%-8% | 4%-6% | 8%-10% | 8%-10% | 4%-6% |
| Ex 6047 | 6%-8% | 4%-6% | 8%-10% | 8%-10% | 6%-8% |
| Ex 6048 | 6%-8% | 4%-6% | 8%-10% | 8%-10% | 8%-10% |
| Ex 6049 | 6%-8% | 6%-8% | 0%-1% | 0%-1% | 0%-1% |
| Ex 6050 | 6%-8% | 6%-8% | 0%-1% | 0%-1% | 1%-2% |
| Ex 6051 | 6%-8% | 6%-8% | 0%-1% | 0%-1% | 2%-4% |
| Ex 6052 | 6%-8% | 6%-8% | 0%-1% | 0%-1% | 4%-6% |
| Ex 6053 | 6%-8% | 6%-8% | 0%-1% | 0%-1% | 6%-8% |
| Ex 6054 | 6%-8% | 6%-8% | 0%-1% | 0%-1% | 8%-10% |
| Ex 6055 | 6%-8% | 6%-8% | 0%-1% | 1%-2% | 0%-1% |
| Ex 6056 | 6%-8% | 6%-8% | 0%-1% | 1%-2% | 1%-2% |
| Ex 6057 | 6%-8% | 6%-8% | 0%-1% | 1%-2% | 2%-4% |
| Ex 6058 | 6%-8% | 6%-8% | 0%-1% | 1%-2% | 4%-6% |
| Ex 6059 | 6%-8% | 6%-8% | 0%-1% | 1%-2% | 6%-8% |
| Ex 6060 | 6%-8% | 6%-8% | 0%-1% | 1%-2% | 8%-10% |
| Ex 6061 | 6%-8% | 6%-8% | 0%-1% | 2%-4% | 0%-1% |
| Ex 6062 | 6%-8% | 6%-8% | 0%-1% | 2%-4% | 1%-2% |
| Ex 6063 | 6%-8% | 6%-8% | 0%-1% | 2%-4% | 2%-4% |
| Ex 6064 | 6%-8% | 6%-8% | 0%-1% | 2%-4% | 4%-6% |
| Ex 6065 | 6%-8% | 6%-8% | 0%-1% | 2%-4% | 6%-8% |
| Ex 6066 | 6%-8% | 6%-8% | 0%-1% | 2%-4% | 8%-10% |
| Ex 6067 | 6%-8% | 6%-8% | 0%-1% | 4%-6% | 0%-1% |
| Ex 6068 | 6%-8% | 6%-8% | 0%-1% | 4%-6% | 1%-2% |
| Ex 6069 | 6%-8% | 6%-8% | 0%-1% | 4%-6% | 2%-4% |
| Ex 6070 | 6%-8% | 6%-8% | 0%-1% | 4%-6% | 4%-6% |
| Ex 6071 | 6%-8% | 6%-8% | 0%-1% | 4%-6% | 6%-8% |
| Ex 6072 | 6%-8% | 6%-8% | 0%-1% | 4%-6% | 8%-10% |
| Ex 6073 | 6%-8% | 6%-8% | 0%-1% | 6%-8% | 0%-1% |
| Ex 6074 | 6%-8% | 6%-8% | 0%-1% | 6%-8% | 1%-2% |
| Ex 6075 | 6%-8% | 6%-8% | 0%-1% | 6%-8% | 2%-4% |
| Ex 6076 | 6%-8% | 6%-8% | 0%-1% | 6%-8% | 4%-6% |
| Ex 6077 | 6%-8% | 6%-8% | 0%-1% | 6%-8% | 6%-8% |
| Ex 6078 | 6%-8% | 6%-8% | 0%-1% | 6%-8% | 8%-10% |
| Ex 6079 | 6%-8% | 6%-8% | 0%-1% | 8%-10% | 0%-1% |
| Ex 6080 | 6%-8% | 6%-8% | 0%-1% | 8%-10% | 1%-2% |
| Ex 6081 | 6%-8% | 6%-8% | 0%-1% | 8%-10% | 2%-4% |
| Ex 6082 | 6%-8% | 6%-8% | 0%-1% | 8%-10% | 4%-6% |
| Ex 6083 | 6%-8% | 6%-8% | 0%-1% | 8%-10% | 6%-8% |
| Ex 6084 | 6%-8% | 6%-8% | 0%-1% | 8%-10% | 8%-10% |
| Ex 6085 | 6%-8% | 6%-8% | 1%-2% | 0%-1% | 0%-1% |
| Ex 6086 | 6%-8% | 6%-8% | 1%-2% | 0%-1% | 1%-2% |
| Ex 6087 | 6%-8% | 6%-8% | 1%-2% | 0%-1% | 2%-4% |
| Ex 6088 | 6%-8% | 6%-8% | 1%-2% | 0%-1% | 4%-6% |
| Ex 6089 | 6%-8% | 6%-8% | 1%-2% | 0%-1% | 6%-8% |
| Ex 6090 | 6%-8% | 6%-8% | 1%-2% | 0%-1% | 8%-10% |
| Ex 6091 | 6%-8% | 6%-8% | 1%-2% | 1%-2% | 0%-1% |
| Ex 6092 | 6%-8% | 6%-8% | 1%-2% | 1%-2% | 1%-2% |
| Ex 6093 | 6%-8% | 6%-8% | 1%-2% | 1%-2% | 2%-4% |
| Ex 6094 | 6%-8% | 6%-8% | 1%-2% | 1%-2% | 4%-6% |
| Ex 6095 | 6%-8% | 6%-8% | 1%-2% | 1%-2% | 6%-8% |
| Ex 6096 | 6%-8% | 6%-8% | 1%-2% | 1%-2% | 8%-10% |
| Ex 6097 | 6%-8% | 6%-8% | 1%-2% | 2%-4% | 0%-1% |
| Ex 6098 | 6%-8% | 6%-8% | 1%-2% | 2%-4% | 1%-2% |
| Ex 6099 | 6%-8% | 6%-8% | 1%-2% | 2%-4% | 2%-4% |
| Ex 6100 | 6%-8% | 6%-8% | 1%-2% | 2%-4% | 4%-6% |
| Ex 6101 | 6%-8% | 6%-8% | 1%-2% | 2%-4% | 6%-8% |
| Ex 6102 | 6%-8% | 6%-8% | 1%-2% | 2%-4% | 8%-10% |
| Ex 6103 | 6%-8% | 6%-8% | 1%-2% | 4%-6% | 0%-1% |
| Ex 6104 | 6%-8% | 6%-8% | 1%-2% | 4%-6% | 1%-2% |
| Ex 6105 | 6%-8% | 6%-8% | 1%-2% | 4%-6% | 2%-4% |
| Ex 6106 | 6%-8% | 6%-8% | 1%-2% | 4%-6% | 4%-6% |
| Ex 6107 | 6%-8% | 6%-8% | 1%-2% | 4%-6% | 6%-8% |
| Ex 6108 | 6%-8% | 6%-8% | 1%-2% | 4%-6% | 8%-10% |
| Ex 6109 | 6%-8% | 6%-8% | 1%-2% | 6%-8% | 0%-1% |
| Ex 6110 | 6%-8% | 6%-8% | 1%-2% | 6%-8% | 1%-2% |
| Ex 6111 | 6%-8% | 6%-8% | 1%-2% | 6%-8% | 2%-4% |
| Ex 6112 | 6%-8% | 6%-8% | 1%-2% | 6%-8% | 4%-6% |
| Ex 6113 | 6%-8% | 6%-8% | 1%-2% | 6%-8% | 6%-8% |
| Ex 6114 | 6%-8% | 6%-8% | 1%-2% | 6%-8% | 8%-10% |
| Ex 6115 | 6%-8% | 6%-8% | 1%-2% | 8%-10% | 0%-1% |
| Ex 6116 | 6%-8% | 6%-8% | 1%-2% | 8%-10% | 1%-2% |
| Ex 6117 | 6%-8% | 6%-8% | 1%-2% | 8%-10% | 2%-4% |
| Ex 6118 | 6%-8% | 6%-8% | 1%-2% | 8%-10% | 4%-6% |
| Ex 6119 | 6%-8% | 6%-8% | 1%-2% | 8%-10% | 6%-8% |
| Ex 6120 | 6%-8% | 6%-8% | 1%-2% | 8%-10% | 8%-10% |
| Ex 6121 | 6%-8% | 6%-8% | 2%-4% | 0%-1% | 0%-1% |
| Ex 6122 | 6%-8% | 6%-8% | 2%-4% | 0%-1% | 1%-2% |
| Ex 6123 | 6%-8% | 6%-8% | 2%-4% | 0%-1% | 2%-4% |
| Ex 6124 | 6%-8% | 6%-8% | 2%-4% | 0%-1% | 4%-6% |
| Ex 6125 | 6%-8% | 6%-8% | 2%-4% | 0%-1% | 6%-8% |
| Ex 6126 | 6%-8% | 6%-8% | 2%-4% | 0%-1% | 8%-10% |
| Ex 6127 | 6%-8% | 6%-8% | 2%-4% | 1%-2% | 0%-1% |
| Ex 6128 | 6%-8% | 6%-8% | 2%-4% | 1%-2% | 1%-2% |
| Ex 6129 | 6%-8% | 6%-8% | 2%-4% | 1%-2% | 2%-4% |
| Ex 6130 | 6%-8% | 6%-8% | 2%-4% | 1%-2% | 4%-6% |
| Ex 6131 | 6%-8% | 6%-8% | 2%-4% | 1%-2% | 6%-8% |
| Ex 6132 | 6%-8% | 6%-8% | 2%-4% | 1%-2% | 8%-10% |
| Ex 6133 | 6%-8% | 6%-8% | 2%-4% | 2%-4% | 0%-1% |
| Ex 6134 | 6%-8% | 6%-8% | 2%-4% | 2%-4% | 1%-2% |
| Ex 6135 | 6%-8% | 6%-8% | 2%-4% | 2%-4% | 2%-4% |
| Ex 6136 | 6%-8% | 6%-8% | 2%-4% | 2%-4% | 4%-6% |
| Ex 6137 | 6%-8% | 6%-8% | 2%-4% | 2%-4% | 6%-8% |
| Ex 6138 | 6%-8% | 6%-8% | 2%-4% | 2%-4% | 8%-10% |
| Ex 6139 | 6%-8% | 6%-8% | 2%-4% | 4%-6% | 0%-1% |
| Ex 6140 | 6%-8% | 6%-8% | 2%-4% | 4%-6% | 1%-2% |
| Ex 6141 | 6%-8% | 6%-8% | 2%-4% | 4%-6% | 2%-4% |
| Ex 6142 | 6%-8% | 6%-8% | 2%-4% | 4%-6% | 4%-6% |
| Ex 6143 | 6%-8% | 6%-8% | 2%-4% | 4%-6% | 6%-8% |
| Ex 6144 | 6%-8% | 6%-8% | 2%-4% | 4%-6% | 8%-10% |
| Ex 6145 | 6%-8% | 6%-8% | 2%-4% | 6%-8% | 0%-1% |
| Ex 6146 | 6%-8% | 6%-8% | 2%-4% | 6%-8% | 1%-2% |
| Ex 6147 | 6%-8% | 6%-8% | 2%-4% | 6%-8% | 2%-4% |
| Ex 6148 | 6%-8% | 6%-8% | 2%-4% | 6%-8% | 4%-6% |
| Ex 6149 | 6%-8% | 6%-8% | 2%-4% | 6%-8% | 6%-8% |
| Ex 6150 | 6%-8% | 6%-8% | 2%-4% | 6%-8% | 8%-10% |
| Ex 6151 | 6%-8% | 6%-8% | 2%-4% | 8%-10% | 0%-1% |
| Ex 6152 | 6%-8% | 6%-8% | 2%-4% | 8%-10% | 1%-2% |
| Ex 6153 | 6%-8% | 6%-8% | 2%-4% | 8%-10% | 2%-4% |
| Ex 6154 | 6%-8% | 6%-8% | 2%-4% | 8%-10% | 4%-6% |
| Ex 6155 | 6%-8% | 6%-8% | 2%-4% | 8%-10% | 6%-8% |
| Ex 6156 | 6%-8% | 6%-8% | 2%-4% | 8%-10% | 8%-10% |
| Ex 6157 | 6%-8% | 6%-8% | 4%-6% | 0%-1% | 0%-1% |
| Ex 6158 | 6%-8% | 6%-8% | 4%-6% | 0%-1% | 1%-2% |
| Ex 6159 | 6%-8% | 6%-8% | 4%-6% | 0%-1% | 2%-4% |
| Ex 6160 | 6%-8% | 6%-8% | 4%-6% | 0%-1% | 4%-6% |
| Ex 6161 | 6%-8% | 6%-8% | 4%-6% | 0%-1% | 6%-8% |
| Ex 6162 | 6%-8% | 6%-8% | 4%-6% | 0%-1% | 8%-10% |
| Ex 6163 | 6%-8% | 6%-8% | 4%-6% | 1%-2% | 0%-1% |
| Ex 6164 | 6%-8% | 6%-8% | 4%-6% | 1%-2% | 1%-2% |
| Ex 6165 | 6%-8% | 6%-8% | 4%-6% | 1%-2% | 2%-4% |
| Ex 6166 | 6%-8% | 6%-8% | 4%-6% | 1%-2% | 4%-6% |
| Ex 6167 | 6%-8% | 6%-8% | 4%-6% | 1%-2% | 6%-8% |
| Ex 6168 | 6%-8% | 6%-8% | 4%-6% | 1%-2% | 8%-10% |
| Ex 6169 | 6%-8% | 6%-8% | 4%-6% | 2%-4% | 0%-1% |
| Ex 6170 | 6%-8% | 6%-8% | 4%-6% | 2%-4% | 1%-2% |
| Ex 6171 | 6%-8% | 6%-8% | 4%-6% | 2%-4% | 2%-4% |
| Ex 6172 | 6%-8% | 6%-8% | 4%-6% | 2%-4% | 4%-6% |
| Ex 6173 | 6%-8% | 6%-8% | 4%-6% | 2%-4% | 6%-8% |
| Ex 6174 | 6%-8% | 6%-8% | 4%-6% | 2%-4% | 8%-10% |
| Ex 6175 | 6%-8% | 6%-8% | 4%-6% | 4%-6% | 0%-1% |
| Ex 6176 | 6%-8% | 6%-8% | 4%-6% | 4%-6% | 1%-2% |
| Ex 6177 | 6%-8% | 6%-8% | 4%-6% | 4%-6% | 2%-4% |
| Ex 6178 | 6%-8% | 6%-8% | 4%-6% | 4%-6% | 4%-6% |
| Ex 6179 | 6%-8% | 6%-8% | 4%-6% | 4%-6% | 6%-8% |
| Ex 6180 | 6%-8% | 6%-8% | 4%-6% | 4%-6% | 8%-10% |
| Ex 6181 | 6%-8% | 6%-8% | 4%-6% | 6%-8% | 0%-1% |
| Ex 6182 | 6%-8% | 6%-8% | 4%-6% | 6%-8% | 1%-2% |
| Ex 6183 | 6%-8% | 6%-8% | 4%-6% | 6%-8% | 2%-4% |
| Ex 6184 | 6%-8% | 6%-8% | 4%-6% | 6%-8% | 4%-6% |
| Ex 6185 | 6%-8% | 6%-8% | 4%-6% | 6%-8% | 6%-8% |
| Ex 6186 | 6%-8% | 6%-8% | 4%-6% | 6%-8% | 8%-10% |
| Ex 6187 | 6%-8% | 6%-8% | 4%-6% | 8%-10% | 0%-1% |
| Ex 6188 | 6%-8% | 6%-8% | 4%-6% | 8%-10% | 1%-2% |
| Ex 6189 | 6%-8% | 6%-8% | 4%-6% | 8%-10% | 2%-4% |
| Ex 6190 | 6%-8% | 6%-8% | 4%-6% | 8%-10% | 4%-6% |
| Ex 6191 | 6%-8% | 6%-8% | 4%-6% | 8%-10% | 6%-8% |
| Ex 6192 | 6%-8% | 6%-8% | 4%-6% | 8%-10% | 8%-10% |
| Ex 6193 | 6%-8% | 6%-8% | 6%-8% | 0%-1% | 0%-1% |
| Ex 6194 | 6%-8% | 6%-8% | 6%-8% | 0%-1% | 1%-2% |
| Ex 6195 | 6%-8% | 6%-8% | 6%-8% | 0%-1% | 2%-4% |
| Ex 6196 | 6%-8% | 6%-8% | 6%-8% | 0%-1% | 4%-6% |
| Ex 6197 | 6%-8% | 6%-8% | 6%-8% | 0%-1% | 6%-8% |
| Ex 6198 | 6%-8% | 6%-8% | 6%-8% | 0%-1% | 8%-10% |
| Ex 6199 | 6%-8% | 6%-8% | 6%-8% | 1%-2% | 0%-1% |
| Ex 6200 | 6%-8% | 6%-8% | 6%-8% | 1%-2% | 1%-2% |
| Ex 6201 | 6%-8% | 6%-8% | 6%-8% | 1%-2% | 2%-4% |
| Ex 6202 | 6%-8% | 6%-8% | 6%-8% | 1%-2% | 4%-6% |
| Ex 6203 | 6%-8% | 6%-8% | 6%-8% | 1%-2% | 6%-8% |
| Ex 6204 | 6%-8% | 6%-8% | 6%-8% | 1%-2% | 8%-10% |
| Ex 6205 | 6%-8% | 6%-8% | 6%-8% | 2%-4% | 0%-1% |
| Ex 6206 | 6%-8% | 6%-8% | 6%-8% | 2%-4% | 1%-2% |
| Ex 6207 | 6%-8% | 6%-8% | 6%-8% | 2%-4% | 2%-4% |
| Ex 6208 | 6%-8% | 6%-8% | 6%-8% | 2%-4% | 4%-6% |
| Ex 6209 | 6%-8% | 6%-8% | 6%-8% | 2%-4% | 6%-8% |
| Ex 6210 | 6%-8% | 6%-8% | 6%-8% | 2%-4% | 8%-10% |
| Ex 6211 | 6%-8% | 6%-8% | 6%-8% | 4%-6% | 0%-1% |
| Ex 6212 | 6%-8% | 6%-8% | 6%-8% | 4%-6% | 1%-2% |
| Ex 6213 | 6%-8% | 6%-8% | 6%-8% | 4%-6% | 2%-4% |
| Ex 6214 | 6%-8% | 6%-8% | 6%-8% | 4%-6% | 4%-6% |
| Ex 6215 | 6%-8% | 6%-8% | 6%-8% | 4%-6% | 6%-8% |
| Ex 6216 | 6%-8% | 6%-8% | 6%-8% | 4%-6% | 8%-10% |
| Ex 6217 | 6%-8% | 6%-8% | 6%-8% | 6%-8% | 0%-1% |
| Ex 6218 | 6%-8% | 6%-8% | 6%-8% | 6%-8% | 1%-2% |
| Ex 6219 | 6%-8% | 6%-8% | 6%-8% | 6%-8% | 2%-4% |
| Ex 6220 | 6%-8% | 6%-8% | 6%-8% | 6%-8% | 4%-6% |
| Ex 6221 | 6%-8% | 6%-8% | 6%-8% | 6%-8% | 6%-8% |
| Ex 6222 | 6%-8% | 6%-8% | 6%-8% | 6%-8% | 8%-10% |
| Ex 6223 | 6%-8% | 6%-8% | 6%-8% | 8%-10% | 0%-1% |
| Ex 6224 | 6%-8% | 6%-8% | 6%-8% | 8%-10% | 1%-2% |
| Ex 6225 | 6%-8% | 6%-8% | 6%-8% | 8%-10% | 2%-4% |
| Ex 6226 | 6%-8% | 6%-8% | 6%-8% | 8%-10% | 4%-6% |
| Ex 6227 | 6%-8% | 6%-8% | 6%-8% | 8%-10% | 6%-8% |
| Ex 6228 | 6%-8% | 6%-8% | 6%-8% | 8%-10% | 8%-10% |
| Ex 6229 | 6%-8% | 6%-8% | 8%-10% | 0%-1% | 0%-1% |
| Ex 6230 | 6%-8% | 6%-8% | 8%-10% | 0%-1% | 1%-2% |
| Ex 6231 | 6%-8% | 6%-8% | 8%-10% | 0%-1% | 2%-4% |
| Ex 6232 | 6%-8% | 6%-8% | 8%-10% | 0%-1% | 4%-6% |
| Ex 6233 | 6%-8% | 6%-8% | 8%-10% | 0%-1% | 6%-8% |
| Ex 6234 | 6%-8% | 6%-8% | 8%-10% | 0%-1% | 8%-10% |
| Ex 6235 | 6%-8% | 6%-8% | 8%-10% | 1%-2% | 0%-1% |
| Ex 6236 | 6%-8% | 6%-8% | 8%-10% | 1%-2% | 1%-2% |
| Ex 6237 | 6%-8% | 6%-8% | 8%-10% | 1%-2% | 2%-4% |
| Ex 6238 | 6%-8% | 6%-8% | 8%-10% | 1%-2% | 4%-6% |
| Ex 6239 | 6%-8% | 6%-8% | 8%-10% | 1%-2% | 6%-8% |
| Ex 6240 | 6%-8% | 6%-8% | 8%-10% | 1%-2% | 8%-10% |
| Ex 6241 | 6%-8% | 6%-8% | 8%-10% | 2%-4% | 0%-1% |
| Ex 6242 | 6%-8% | 6%-8% | 8%-10% | 2%-4% | 1%-2% |
| Ex 6243 | 6%-8% | 6%-8% | 8%-10% | 2%-4% | 2%-4% |
| Ex 6244 | 6%-8% | 6%-8% | 8%-10% | 2%-4% | 4%-6% |
| Ex 6245 | 6%-8% | 6%-8% | 8%-10% | 2%-4% | 6%-8% |
| Ex 6246 | 6%-8% | 6%-8% | 8%-10% | 2%-4% | 8%-10% |
| Ex 6247 | 6%-8% | 6%-8% | 8%-10% | 4%-6% | 0%-1% |
| Ex 6248 | 6%-8% | 6%-8% | 8%-10% | 4%-6% | 1%-2% |
| Ex 6249 | 6%-8% | 6%-8% | 8%-10% | 4%-6% | 2%-4% |
| Ex 6250 | 6%-8% | 6%-8% | 8%-10% | 4%-6% | 4%-6% |
| Ex 6251 | 6%-8% | 6%-8% | 8%-10% | 4%-6% | 6%-8% |
| Ex 6252 | 6%-8% | 6%-8% | 8%-10% | 4%-6% | 8%-10% |
| Ex 6253 | 6%-8% | 6%-8% | 8%-10% | 6%-8% | 0%-1% |
| Ex 6254 | 6%-8% | 6%-8% | 8%-10% | 6%-8% | 1%-2% |
| Ex 6255 | 6%-8% | 6%-8% | 8%-10% | 6%-8% | 2%-4% |
| Ex 6256 | 6%-8% | 6%-8% | 8%-10% | 6%-8% | 4%-6% |
| Ex 6257 | 6%-8% | 6%-8% | 8%-10% | 6%-8% | 6%-8% |
| Ex 6258 | 6%-8% | 6%-8% | 8%-10% | 6%-8% | 8%-10% |
| Ex 6259 | 6%-8% | 6%-8% | 8%-10% | 8%-10% | 0%-1% |
| Ex 6260 | 6%-8% | 6%-8% | 8%-10% | 8%-10% | 1%-2% |
| Ex 6261 | 6%-8% | 6%-8% | 8%-10% | 8%-10% | 2%-4% |
| Ex 6262 | 6%-8% | 6%-8% | 8%-10% | 8%-10% | 4%-6% |
| Ex 6263 | 6%-8% | 6%-8% | 8%-10% | 8%-10% | 6%-8% |
| Ex 6264 | 6%-8% | 6%-8% | 8%-10% | 8%-10% | 8%-10% |
| Ex 6265 | 6%-8% | 8%-10% | 0%-1% | 0%-1% | 0%-1% |
| Ex 6266 | 6%-8% | 8%-10% | 0%-1% | 0%-1% | 1%-2% |
| Ex 6267 | 6%-8% | 8%-10% | 0%-1% | 0%-1% | 2%-4% |
| Ex 6268 | 6%-8% | 8%-10% | 0%-1% | 0%-1% | 4%-6% |
| Ex 6269 | 6%-8% | 8%-10% | 0%-1% | 0%-1% | 6%-8% |
| Ex 6270 | 6%-8% | 8%-10% | 0%-1% | 0%-1% | 8%-10% |
| Ex 6271 | 6%-8% | 8%-10% | 0%-1% | 1%-2% | 0%-1% |
| Ex 6272 | 6%-8% | 8%-10% | 0%-1% | 1%-2% | 1%-2% |
| Ex 6273 | 6%-8% | 8%-10% | 0%-1% | 1%-2% | 2%-4% |
| Ex 6274 | 6%-8% | 8%-10% | 0%-1% | 1%-2% | 4%-6% |
| Ex 6275 | 6%-8% | 8%-10% | 0%-1% | 1%-2% | 6%-8% |
| Ex 6276 | 6%-8% | 8%-10% | 0%-1% | 1%-2% | 8%-10% |
| Ex 6277 | 6%-8% | 8%-10% | 0%-1% | 2%-4% | 0%-1% |
| Ex 6278 | 6%-8% | 8%-10% | 0%-1% | 2%-4% | 1%-2% |
| Ex 6279 | 6%-8% | 8%-10% | 0%-1% | 2%-4% | 2%-4% |
| Ex 6280 | 6%-8% | 8%-10% | 0%-1% | 2%-4% | 4%-6% |
| Ex 6281 | 6%-8% | 8%-10% | 0%-1% | 2%-4% | 6%-8% |
| Ex 6282 | 6%-8% | 8%-10% | 0%-1% | 2%-4% | 8%-10% |
| Ex 6283 | 6%-8% | 8%-10% | 0%-1% | 4%-6% | 0%-1% |
| Ex 6284 | 6%-8% | 8%-10% | 0%-1% | 4%-6% | 1%-2% |
| Ex 6285 | 6%-8% | 8%-10% | 0%-1% | 4%-6% | 2%-4% |
| Ex 6286 | 6%-8% | 8%-10% | 0%-1% | 4%-6% | 4%-6% |
| Ex 6287 | 6%-8% | 8%-10% | 0%-1% | 4%-6% | 6%-8% |
| Ex 6288 | 6%-8% | 8%-10% | 0%-1% | 4%-6% | 8%-10% |
| Ex 6289 | 6%-8% | 8%-10% | 0%-1% | 6%-8% | 0%-1% |
| Ex 6290 | 6%-8% | 8%-10% | 0%-1% | 6%-8% | 1%-2% |
| Ex 6291 | 6%-8% | 8%-10% | 0%-1% | 6%-8% | 2%-4% |
| Ex 6292 | 6%-8% | 8%-10% | 0%-1% | 6%-8% | 4%-6% |
| Ex 6293 | 6%-8% | 8%-10% | 0%-1% | 6%-8% | 6%-8% |
| Ex 6294 | 6%-8% | 8%-10% | 0%-1% | 6%-8% | 8%-10% |
| Ex 6295 | 6%-8% | 8%-10% | 0%-1% | 8%-10% | 0%-1% |
| Ex 6296 | 6%-8% | 8%-10% | 0%-1% | 8%-10% | 1%-2% |
| Ex 6297 | 6%-8% | 8%-10% | 0%-1% | 8%-10% | 2%-4% |
| Ex 6298 | 6%-8% | 8%-10% | 0%-1% | 8%-10% | 4%-6% |
| Ex 6299 | 6%-8% | 8%-10% | 0%-1% | 8%-10% | 6%-8% |
| Ex 6300 | 6%-8% | 8%-10% | 0%-1% | 8%-10% | 8%-10% |
| Ex 6301 | 6%-8% | 8%-10% | 1%-2% | 0%-1% | 0%-1% |
| Ex 6302 | 6%-8% | 8%-10% | 1%-2% | 0%-1% | 1%-2% |
| Ex 6303 | 6%-8% | 8%-10% | 1%-2% | 0%-1% | 2%-4% |
| Ex 6304 | 6%-8% | 8%-10% | 1%-2% | 0%-1% | 4%-6% |
| Ex 6305 | 6%-8% | 8%-10% | 1%-2% | 0%-1% | 6%-8% |
| Ex 6306 | 6%-8% | 8%-10% | 1%-2% | 0%-1% | 8%-10% |
| Ex 6307 | 6%-8% | 8%-10% | 1%-2% | 1%-2% | 0%-1% |
| Ex 6308 | 6%-8% | 8%-10% | 1%-2% | 1%-2% | 1%-2% |
| Ex 6309 | 6%-8% | 8%-10% | 1%-2% | 1%-2% | 2%-4% |
| Ex 6310 | 6%-8% | 8%-10% | 1%-2% | 1%-2% | 4%-6% |
| Ex 6311 | 6%-8% | 8%-10% | 1%-2% | 1%-2% | 6%-8% |
| Ex 6312 | 6%-8% | 8%-10% | 1%-2% | 1%-2% | 8%-10% |
| Ex 6313 | 6%-8% | 8%-10% | 1%-2% | 2%-4% | 0%-1% |
| Ex 6314 | 6%-8% | 8%-10% | 1%-2% | 2%-4% | 1%-2% |
| Ex 6315 | 6%-8% | 8%-10% | 1%-2% | 2%-4% | 2%-4% |
| Ex 6316 | 6%-8% | 8%-10% | 1%-2% | 2%-4% | 4%-6% |
| Ex 6317 | 6%-8% | 8%-10% | 1%-2% | 2%-4% | 6%-8% |
| Ex 6318 | 6%-8% | 8%-10% | 1%-2% | 2%-4% | 8%-10% |
| Ex 6319 | 6%-8% | 8%-10% | 1%-2% | 4%-6% | 0%-1% |
| Ex 6320 | 6%-8% | 8%-10% | 1%-2% | 4%-6% | 1%-2% |
| Ex 6321 | 6%-8% | 8%-10% | 1%-2% | 4%-6% | 2%-4% |
| Ex 6322 | 6%-8% | 8%-10% | 1%-2% | 4%-6% | 4%-6% |
| Ex 6323 | 6%-8% | 8%-10% | 1%-2% | 4%-6% | 6%-8% |
| Ex 6324 | 6%-8% | 8%-10% | 1%-2% | 4%-6% | 8%-10% |
| Ex 6325 | 6%-8% | 8%-10% | 1%-2% | 6%-8% | 0%-1% |
| Ex 6326 | 6%-8% | 8%-10% | 1%-2% | 6%-8% | 1%-2% |
| Ex 6327 | 6%-8% | 8%-10% | 1%-2% | 6%-8% | 2%-4% |
| Ex 6328 | 6%-8% | 8%-10% | 1%-2% | 6%-8% | 4%-6% |
| Ex 6329 | 6%-8% | 8%-10% | 1%-2% | 6%-8% | 6%-8% |
| Ex 6330 | 6%-8% | 8%-10% | 1%-2% | 6%-8% | 8%-10% |
| Ex 6331 | 6%-8% | 8%-10% | 1%-2% | 8%-10% | 0%-1% |
| Ex 6332 | 6%-8% | 8%-10% | 1%-2% | 8%-10% | 1%-2% |
| Ex 6333 | 6%-8% | 8%-10% | 1%-2% | 8%-10% | 2%-4% |
| Ex 6334 | 6%-8% | 8%-10% | 1%-2% | 8%-10% | 4%-6% |
| Ex 6335 | 6%-8% | 8%-10% | 1%-2% | 8%-10% | 6%-8% |
| Ex 6336 | 6%-8% | 8%-10% | 1%-2% | 8%-10% | 8%-10% |
| Ex 6337 | 6%-8% | 8%-10% | 2%-4% | 0%-1% | 0%-1% |
| Ex 6338 | 6%-8% | 8%-10% | 2%-4% | 0%-1% | 1%-2% |
| Ex 6339 | 6%-8% | 8%-10% | 2%-4% | 0%-1% | 2%-4% |
| Ex 6340 | 6%-8% | 8%-10% | 2%-4% | 0%-1% | 4%-6% |
| Ex 6341 | 6%-8% | 8%-10% | 2%-4% | 0%-1% | 6%-8% |
| Ex 6342 | 6%-8% | 8%-10% | 2%-4% | 0%-1% | 8%-10% |
| Ex 6343 | 6%-8% | 8%-10% | 2%-4% | 1%-2% | 0%-1% |
| Ex 6344 | 6%-8% | 8%-10% | 2%-4% | 1%-2% | 1%-2% |
| Ex 6345 | 6%-8% | 8%-10% | 2%-4% | 1%-2% | 2%-4% |
| Ex 6346 | 6%-8% | 8%-10% | 2%-4% | 1%-2% | 4%-6% |
| Ex 6347 | 6%-8% | 8%-10% | 2%-4% | 1%-2% | 6%-8% |
| Ex 6348 | 6%-8% | 8%-10% | 2%-4% | 1%-2% | 8%-10% |
| Ex 6349 | 6%-8% | 8%-10% | 2%-4% | 2%-4% | 0%-1% |
| Ex 6350 | 6%-8% | 8%-10% | 2%-4% | 2%-4% | 1%-2% |
| Ex 6351 | 6%-8% | 8%-10% | 2%-4% | 2%-4% | 2%-4% |
| Ex 6352 | 6%-8% | 8%-10% | 2%-4% | 2%-4% | 4%-6% |
| Ex 6353 | 6%-8% | 8%-10% | 2%-4% | 2%-4% | 6%-8% |
| Ex 6354 | 6%-8% | 8%-10% | 2%-4% | 2%-4% | 8%-10% |
| Ex 6355 | 6%-8% | 8%-10% | 2%-4% | 4%-6% | 0%-1% |
| Ex 6356 | 6%-8% | 8%-10% | 2%-4% | 4%-6% | 1%-2% |
| Ex 6357 | 6%-8% | 8%-10% | 2%-4% | 4%-6% | 2%-4% |
| Ex 6358 | 6%-8% | 8%-10% | 2%-4% | 4%-6% | 4%-6% |
| Ex 6359 | 6%-8% | 8%-10% | 2%-4% | 4%-6% | 6%-8% |
| Ex 6360 | 6%-8% | 8%-10% | 2%-4% | 4%-6% | 8%-10% |
| Ex 6361 | 6%-8% | 8%-10% | 2%-4% | 6%-8% | 0%-1% |
| Ex 6362 | 6%-8% | 8%-10% | 2%-4% | 6%-8% | 1%-2% |
| Ex 6363 | 6%-8% | 8%-10% | 2%-4% | 6%-8% | 2%-4% |
| Ex 6364 | 6%-8% | 8%-10% | 2%-4% | 6%-8% | 4%-6% |
| Ex 6365 | 6%-8% | 8%-10% | 2%-4% | 6%-8% | 6%-8% |
| Ex 6366 | 6%-8% | 8%-10% | 2%-4% | 6%-8% | 8%-10% |
| Ex 6367 | 6%-8% | 8%-10% | 2%-4% | 8%-10% | 0%-1% |
| Ex 6368 | 6%-8% | 8%-10% | 2%-4% | 8%-10% | 1%-2% |
| Ex 6369 | 6%-8% | 8%-10% | 2%-4% | 8%-10% | 2%-4% |
| Ex 6370 | 6%-8% | 8%-10% | 2%-4% | 8%-10% | 4%-6% |
| Ex 6371 | 6%-8% | 8%-10% | 2%-4% | 8%-10% | 6%-8% |
| Ex 6372 | 6%-8% | 8%-10% | 2%-4% | 8%-10% | 8%-10% |
| Ex 6373 | 6%-8% | 8%-10% | 4%-6% | 0%-1% | 0%-1% |
| Ex 6374 | 6%-8% | 8%-10% | 4%-6% | 0%-1% | 1%-2% |
| Ex 6375 | 6%-8% | 8%-10% | 4%-6% | 0%-1% | 2%-4% |
| Ex 6376 | 6%-8% | 8%-10% | 4%-6% | 0%-1% | 4%-6% |
| Ex 6377 | 6%-8% | 8%-10% | 4%-6% | 0%-1% | 6%-8% |
| Ex 6378 | 6%-8% | 8%-10% | 4%-6% | 0%-1% | 8%-10% |
| Ex 6379 | 6%-8% | 8%-10% | 4%-6% | 1%-2% | 0%-1% |
| Ex 6380 | 6%-8% | 8%-10% | 4%-6% | 1%-2% | 1%-2% |
| Ex 6381 | 6%-8% | 8%-10% | 4%-6% | 1%-2% | 2%-4% |
| Ex 6382 | 6%-8% | 8%-10% | 4%-6% | 1%-2% | 4%-6% |
| Ex 6383 | 6%-8% | 8%-10% | 4%-6% | 1%-2% | 6%-8% |
| Ex 6384 | 6%-8% | 8%-10% | 4%-6% | 1%-2% | 8%-10% |
| Ex 6385 | 6%-8% | 8%-10% | 4%-6% | 2%-4% | 0%-1% |
| Ex 6386 | 6%-8% | 8%-10% | 4%-6% | 2%-4% | 1%-2% |
| Ex 6387 | 6%-8% | 8%-10% | 4%-6% | 2%-4% | 2%-4% |
| Ex 6388 | 6%-8% | 8%-10% | 4%-6% | 2%-4% | 4%-6% |
| Ex 6389 | 6%-8% | 8%-10% | 4%-6% | 2%-4% | 6%-8% |
| Ex 6390 | 6%-8% | 8%-10% | 4%-6% | 2%-4% | 8%-10% |
| Ex 6391 | 6%-8% | 8%-10% | 4%-6% | 4%-6% | 0%-1% |
| Ex 6392 | 6%-8% | 8%-10% | 4%-6% | 4%-6% | 1%-2% |
| Ex 6393 | 6%-8% | 8%-10% | 4%-6% | 4%-6% | 2%-4% |
| Ex 6394 | 6%-8% | 8%-10% | 4%-6% | 4%-6% | 4%-6% |
| Ex 6395 | 6%-8% | 8%-10% | 4%-6% | 4%-6% | 6%-8% |
| Ex 6396 | 6%-8% | 8%-10% | 4%-6% | 4%-6% | 8%-10% |
| Ex 6397 | 6%-8% | 8%-10% | 4%-6% | 6%-8% | 0%-1% |
| Ex 6398 | 6%-8% | 8%-10% | 4%-6% | 6%-8% | 1%-2% |
| Ex 6399 | 6%-8% | 8%-10% | 4%-6% | 6%-8% | 2%-4% |
| Ex 6400 | 6%-8% | 8%-10% | 4%-6% | 6%-8% | 4%-6% |
| Ex 6401 | 6%-8% | 8%-10% | 4%-6% | 6%-8% | 6%-8% |
| Ex 6402 | 6%-8% | 8%-10% | 4%-6% | 6%-8% | 8%-10% |
| Ex 6403 | 6%-8% | 8%-10% | 4%-6% | 8%-10% | 0%-1% |
| Ex 6404 | 6%-8% | 8%-10% | 4%-6% | 8%-10% | 1%-2% |
| Ex 6405 | 6%-8% | 8%-10% | 4%-6% | 8%-10% | 2%-4% |
| Ex 6406 | 6%-8% | 8%-10% | 4%-6% | 8%-10% | 4%-6% |
| Ex 6407 | 6%-8% | 8%-10% | 4%-6% | 8%-10% | 6%-8% |
| Ex 6408 | 6%-8% | 8%-10% | 4%-6% | 8%-10% | 8%-10% |
| Ex 6409 | 6%-8% | 8%-10% | 6%-8% | 0%-1% | 0%-1% |
| Ex 6410 | 6%-8% | 8%-10% | 6%-8% | 0%-1% | 1%-2% |
| Ex 6411 | 6%-8% | 8%-10% | 6%-8% | 0%-1% | 2%-4% |
| Ex 6412 | 6%-8% | 8%-10% | 6%-8% | 0%-1% | 4%-6% |
| Ex 6413 | 6%-8% | 8%-10% | 6%-8% | 0%-1% | 6%-8% |
| Ex 6414 | 6%-8% | 8%-10% | 6%-8% | 0%-1% | 8%-10% |
| Ex 6415 | 6%-8% | 8%-10% | 6%-8% | 1%-2% | 0%-1% |
| Ex 6416 | 6%-8% | 8%-10% | 6%-8% | 1%-2% | 1%-2% |
| Ex 6417 | 6%-8% | 8%-10% | 6%-8% | 1%-2% | 2%-4% |
| Ex 6418 | 6%-8% | 8%-10% | 6%-8% | 1%-2% | 4%-6% |
| Ex 6419 | 6%-8% | 8%-10% | 6%-8% | 1%-2% | 6%-8% |
| Ex 6420 | 6%-8% | 8%-10% | 6%-8% | 1%-2% | 8%-10% |
| Ex 6421 | 6%-8% | 8%-10% | 6%-8% | 2%-4% | 0%-1% |
| Ex 6422 | 6%-8% | 8%-10% | 6%-8% | 2%-4% | 1%-2% |
| Ex 6423 | 6%-8% | 8%-10% | 6%-8% | 2%-4% | 2%-4% |
| Ex 6424 | 6%-8% | 8%-10% | 6%-8% | 2%-4% | 4%-6% |
| Ex 6425 | 6%-8% | 8%-10% | 6%-8% | 2%-4% | 6%-8% |
| Ex 6426 | 6%-8% | 8%-10% | 6%-8% | 2%-4% | 8%-10% |
| Ex 6427 | 6%-8% | 8%-10% | 6%-8% | 4%-6% | 0%-1% |
| Ex 6428 | 6%-8% | 8%-10% | 6%-8% | 4%-6% | 1%-2% |
| Ex 6429 | 6%-8% | 8%-10% | 6%-8% | 4%-6% | 2%-4% |
| Ex 6430 | 6%-8% | 8%-10% | 6%-8% | 4%-6% | 4%-6% |
| Ex 6431 | 6%-8% | 8%-10% | 6%-8% | 4%-6% | 6%-8% |
| Ex 6432 | 6%-8% | 8%-10% | 6%-8% | 4%-6% | 8%-10% |
| Ex 6433 | 6%-8% | 8%-10% | 6%-8% | 6%-8% | 0%-1% |
| Ex 6434 | 6%-8% | 8%-10% | 6%-8% | 6%-8% | 1%-2% |
| Ex 6435 | 6%-8% | 8%-10% | 6%-8% | 6%-8% | 2%-4% |
| Ex 6436 | 6%-8% | 8%-10% | 6%-8% | 6%-8% | 4%-6% |
| Ex 6437 | 6%-8% | 8%-10% | 6%-8% | 6%-8% | 6%-8% |
| Ex 6438 | 6%-8% | 8%-10% | 6%-8% | 6%-8% | 8%-10% |
| Ex 6439 | 6%-8% | 8%-10% | 6%-8% | 8%-10% | 0%-1% |
| Ex 6440 | 6%-8% | 8%-10% | 6%-8% | 8%-10% | 1%-2% |
| Ex 6441 | 6%-8% | 8%-10% | 6%-8% | 8%-10% | 2%-4% |
| Ex 6442 | 6%-8% | 8%-10% | 6%-8% | 8%-10% | 4%-6% |
| Ex 6443 | 6%-8% | 8%-10% | 6%-8% | 8%-10% | 6%-8% |
| Ex 6444 | 6%-8% | 8%-10% | 6%-8% | 8%-10% | 8%-10% |
| Ex 6445 | 6%-8% | 8%-10% | 8%-10% | 0%-1% | 0%-1% |
| Ex 6446 | 6%-8% | 8%-10% | 8%-10% | 0%-1% | 1%-2% |
| Ex 6447 | 6%-8% | 8%-10% | 8%-10% | 0%-1% | 2%-4% |
| Ex 6448 | 6%-8% | 8%-10% | 8%-10% | 0%-1% | 4%-6% |
| Ex 6449 | 6%-8% | 8%-10% | 8%-10% | 0%-1% | 6%-8% |
| Ex 6450 | 6%-8% | 8%-10% | 8%-10% | 0%-1% | 8%-10% |
| Ex 6451 | 6%-8% | 8%-10% | 8%-10% | 1%-2% | 0%-1% |
| Ex 6452 | 6%-8% | 8%-10% | 8%-10% | 1%-2% | 1%-2% |
| Ex 6453 | 6%-8% | 8%-10% | 8%-10% | 1%-2% | 2%-4% |
| Ex 6454 | 6%-8% | 8%-10% | 8%-10% | 1%-2% | 4%-6% |
| Ex 6455 | 6%-8% | 8%-10% | 8%-10% | 1%-2% | 6%-8% |
| Ex 6456 | 6%-8% | 8%-10% | 8%-10% | 1%-2% | 8%-10% |
| Ex 6457 | 6%-8% | 8%-10% | 8%-10% | 2%-4% | 0%-1% |
| Ex 6458 | 6%-8% | 8%-10% | 8%-10% | 2%-4% | 1%-2% |
| Ex 6459 | 6%-8% | 8%-10% | 8%-10% | 2%-4% | 2%-4% |
| Ex 6460 | 6%-8% | 8%-10% | 8%-10% | 2%-4% | 4%-6% |
| Ex 6461 | 6%-8% | 8%-10% | 8%-10% | 2%-4% | 6%-8% |
| Ex 6462 | 6%-8% | 8%-10% | 8%-10% | 2%-4% | 8%-10% |
| Ex 6463 | 6%-8% | 8%-10% | 8%-10% | 4%-6% | 0%-1% |
| Ex 6464 | 6%-8% | 8%-10% | 8%-10% | 4%-6% | 1%-2% |
| Ex 6465 | 6%-8% | 8%-10% | 8%-10% | 4%-6% | 2%-4% |
| Ex 6466 | 6%-8% | 8%-10% | 8%-10% | 4%-6% | 4%-6% |
| Ex 6467 | 6%-8% | 8%-10% | 8%-10% | 4%-6% | 6%-8% |
| Ex 6468 | 6%-8% | 8%-10% | 8%-10% | 4%-6% | 8%-10% |
| Ex 6469 | 6%-8% | 8%-10% | 8%-10% | 6%-8% | 0%-1% |
| Ex 6470 | 6%-8% | 8%-10% | 8%-10% | 6%-8% | 1%-2% |
| Ex 6471 | 6%-8% | 8%-10% | 8%-10% | 6%-8% | 2%-4% |
| Ex 6472 | 6%-8% | 8%-10% | 8%-10% | 6%-8% | 4%-6% |
| Ex 6473 | 6%-8% | 8%-10% | 8%-10% | 6%-8% | 6%-8% |
| Ex 6474 | 6%-8% | 8%-10% | 8%-10% | 6%-8% | 8%-10% |
| Ex 6475 | 6%-8% | 8%-10% | 8%-10% | 8%-10% | 0%-1% |
| Ex 6476 | 6%-8% | 8%-10% | 8%-10% | 8%-10% | 1%-2% |
| Ex 6477 | 6%-8% | 8%-10% | 8%-10% | 8%-10% | 2%-4% |
| Ex 6478 | 6%-8% | 8%-10% | 8%-10% | 8%-10% | 4%-6% |
| Ex 6479 | 6%-8% | 8%-10% | 8%-10% | 8%-10% | 6%-8% |
| Ex 6480 | 6%-8% | 8%-10% | 8%-10% | 8%-10% | 8%-10% |
| Ex 6481 | 8%-10% | 0%-1% | 0%-1% | 0%-1% | 0%-1% |
| Ex 6482 | 8%-10% | 0%-1% | 0%-1% | 0%-1% | 1%-2% |
| Ex 6483 | 8%-10% | 0%-1% | 0%-1% | 0%-1% | 2%-4% |
| Ex 6484 | 8%-10% | 0%-1% | 0%-1% | 0%-1% | 4%-6% |
| Ex 6485 | 8%-10% | 0%-1% | 0%-1% | 0%-1% | 6%-8% |
| Ex 6486 | 8%-10% | 0%-1% | 0%-1% | 0%-1% | 8%-10% |
| Ex 6487 | 8%-10% | 0%-1% | 0%-1% | 1%-2% | 0%-1% |
| Ex 6488 | 8%-10% | 0%-1% | 0%-1% | 1%-2% | 1%-2% |
| Ex 6489 | 8%-10% | 0%-1% | 0%-1% | 1%-2% | 2%-4% |
| Ex 6490 | 8%-10% | 0%-1% | 0%-1% | 1%-2% | 4%-6% |
| Ex 6491 | 8%-10% | 0%-1% | 0%-1% | 1%-2% | 6%-8% |
| Ex 6492 | 8%-10% | 0%-1% | 0%-1% | 1%-2% | 8%-10% |
| Ex 6493 | 8%-10% | 0%-1% | 0%-1% | 2%-4% | 0%-1% |
| Ex 6494 | 8%-10% | 0%-1% | 0%-1% | 2%-4% | 1%-2% |
| Ex 6495 | 8%-10% | 0%-1% | 0%-1% | 2%-4% | 2%-4% |
| Ex 6496 | 8%-10% | 0%-1% | 0%-1% | 2%-4% | 4%-6% |
| Ex 6497 | 8%-10% | 0%-1% | 0%-1% | 2%-4% | 6%-8% |
| Ex 6498 | 8%-10% | 0%-1% | 0%-1% | 2%-4% | 8%-10% |
| Ex 6499 | 8%-10% | 0%-1% | 0%-1% | 4%-6% | 0%-1% |
| Ex 6500 | 8%-10% | 0%-1% | 0%-1% | 4%-6% | 1%-2% |
| Ex 6501 | 8%-10% | 0%-1% | 0%-1% | 4%-6% | 2%-4% |
| Ex 6502 | 8%-10% | 0%-1% | 0%-1% | 4%-6% | 4%-6% |
| Ex 6503 | 8%-10% | 0%-1% | 0%-1% | 4%-6% | 6%-8% |
| Ex 6504 | 8%-10% | 0%-1% | 0%-1% | 4%-6% | 8%-10% |
| Ex 6505 | 8%-10% | 0%-1% | 0%-1% | 6%-8% | 0%-1% |
| Ex 6506 | 8%-10% | 0%-1% | 0%-1% | 6%-8% | 1%-2% |
| Ex 6507 | 8%-10% | 0%-1% | 0%-1% | 6%-8% | 2%-4% |
| Ex 6508 | 8%-10% | 0%-1% | 0%-1% | 6%-8% | 4%-6% |
| Ex 6509 | 8%-10% | 0%-1% | 0%-1% | 6%-8% | 6%-8% |
| Ex 6510 | 8%-10% | 0%-1% | 0%-1% | 6%-8% | 8%-10% |
| Ex 6511 | 8%-10% | 0%-1% | 0%-1% | 8%-10% | 0%-1% |
| Ex 6512 | 8%-10% | 0%-1% | 0%-1% | 8%-10% | 1%-2% |
| Ex 6513 | 8%-10% | 0%-1% | 0%-1% | 8%-10% | 2%-4% |
| Ex 6514 | 8%-10% | 0%-1% | 0%-1% | 8%-10% | 4%-6% |
| Ex 6515 | 8%-10% | 0%-1% | 0%-1% | 8%-10% | 6%-8% |
| Ex 6516 | 8%-10% | 0%-1% | 0%-1% | 8%-10% | 8%-10% |
| Ex 6517 | 8%-10% | 0%-1% | 1%-2% | 0%-1% | 0%-1% |
| Ex 6518 | 8%-10% | 0%-1% | 1%-2% | 0%-1% | 1%-2% |
| Ex 6519 | 8%-10% | 0%-1% | 1%-2% | 0%-1% | 2%-4% |
| Ex 6520 | 8%-10% | 0%-1% | 1%-2% | 0%-1% | 4%-6% |
| Ex 6521 | 8%-10% | 0%-1% | 1%-2% | 0%-1% | 6%-8% |
| Ex 6522 | 8%-10% | 0%-1% | 1%-2% | 0%-1% | 8%-10% |
| Ex 6523 | 8%-10% | 0%-1% | 1%-2% | 1%-2% | 0%-1% |
| Ex 6524 | 8%-10% | 0%-1% | 1%-2% | 1%-2% | 1%-2% |
| Ex 6525 | 8%-10% | 0%-1% | 1%-2% | 1%-2% | 2%-4% |
| Ex 6526 | 8%-10% | 0%-1% | 1%-2% | 1%-2% | 4%-6% |
| Ex 6527 | 8%-10% | 0%-1% | 1%-2% | 1%-2% | 6%-8% |
| Ex 6528 | 8%-10% | 0%-1% | 1%-2% | 1%-2% | 8%-10% |
| Ex 6529 | 8%-10% | 0%-1% | 1%-2% | 2%-4% | 0%-1% |
| Ex 6530 | 8%-10% | 0%-1% | 1%-2% | 2%-4% | 1%-2% |
| Ex 6531 | 8%-10% | 0%-1% | 1%-2% | 2%-4% | 2%-4% |
| Ex 6532 | 8%-10% | 0%-1% | 1%-2% | 2%-4% | 4%-6% |
| Ex 6533 | 8%-10% | 0%-1% | 1%-2% | 2%-4% | 6%-8% |
| Ex 6534 | 8%-10% | 0%-1% | 1%-2% | 2%-4% | 8%-10% |
| Ex 6535 | 8%-10% | 0%-1% | 1%-2% | 4%-6% | 0%-1% |
| Ex 6536 | 8%-10% | 0%-1% | 1%-2% | 4%-6% | 1%-2% |
| Ex 6537 | 8%-10% | 0%-1% | 1%-2% | 4%-6% | 2%-4% |
| Ex 6538 | 8%-10% | 0%-1% | 1%-2% | 4%-6% | 4%-6% |
| Ex 6539 | 8%-10% | 0%-1% | 1%-2% | 4%-6% | 6%-8% |
| Ex 6540 | 8%-10% | 0%-1% | 1%-2% | 4%-6% | 8%-10% |
| Ex 6541 | 8%-10% | 0%-1% | 1%-2% | 6%-8% | 0%-1% |
| Ex 6542 | 8%-10% | 0%-1% | 1%-2% | 6%-8% | 1%-2% |
| Ex 6543 | 8%-10% | 0%-1% | 1%-2% | 6%-8% | 2%-4% |
| Ex 6544 | 8%-10% | 0%-1% | 1%-2% | 6%-8% | 4%-6% |
| Ex 6545 | 8%-10% | 0%-1% | 1%-2% | 6%-8% | 6%-8% |
| Ex 6546 | 8%-10% | 0%-1% | 1%-2% | 6%-8% | 8%-10% |
| Ex 6547 | 8%-10% | 0%-1% | 1%-2% | 8%-10% | 0%-1% |
| Ex 6548 | 8%-10% | 0%-1% | 1%-2% | 8%-10% | 1%-2% |
| Ex 6549 | 8%-10% | 0%-1% | 1%-2% | 8%-10% | 2%-4% |
| Ex 6550 | 8%-10% | 0%-1% | 1%-2% | 8%-10% | 4%-6% |
| Ex 6551 | 8%-10% | 0%-1% | 1%-2% | 8%-10% | 6%-8% |
| Ex 6552 | 8%-10% | 0%-1% | 1%-2% | 8%-10% | 8%-10% |
| Ex 6553 | 8%-10% | 0%-1% | 2%-4% | 0%-1% | 0%-1% |
| Ex 6554 | 8%-10% | 0%-1% | 2%-4% | 0%-1% | 1%-2% |
| Ex 6555 | 8%-10% | 0%-1% | 2%-4% | 0%-1% | 2%-4% |
| Ex 6556 | 8%-10% | 0%-1% | 2%-4% | 0%-1% | 4%-6% |
| Ex 6557 | 8%-10% | 0%-1% | 2%-4% | 0%-1% | 6%-8% |
| Ex 6558 | 8%-10% | 0%-1% | 2%-4% | 0%-1% | 8%-10% |
| Ex 6559 | 8%-10% | 0%-1% | 2%-4% | 1%-2% | 0%-1% |
| Ex 6560 | 8%-10% | 0%-1% | 2%-4% | 1%-2% | 1%-2% |
| Ex 6561 | 8%-10% | 0%-1% | 2%-4% | 1%-2% | 2%-4% |
| Ex 6562 | 8%-10% | 0%-1% | 2%-4% | 1%-2% | 4%-6% |
| Ex 6563 | 8%-10% | 0%-1% | 2%-4% | 1%-2% | 6%-8% |
| Ex 6564 | 8%-10% | 0%-1% | 2%-4% | 1%-2% | 8%-10% |
| Ex 6565 | 8%-10% | 0%-1% | 2%-4% | 2%-4% | 0%-1% |
| Ex 6566 | 8%-10% | 0%-1% | 2%-4% | 2%-4% | 1%-2% |
| Ex 6567 | 8%-10% | 0%-1% | 2%-4% | 2%-4% | 2%-4% |
| Ex 6568 | 8%-10% | 0%-1% | 2%-4% | 2%-4% | 4%-6% |
| Ex 6569 | 8%-10% | 0%-1% | 2%-4% | 2%-4% | 6%-8% |
| Ex 6570 | 8%-10% | 0%-1% | 2%-4% | 2%-4% | 8%-10% |
| Ex 6571 | 8%-10% | 0%-1% | 2%-4% | 4%-6% | 0%-1% |
| Ex 6572 | 8%-10% | 0%-1% | 2%-4% | 4%-6% | 1%-2% |
| Ex 6573 | 8%-10% | 0%-1% | 2%-4% | 4%-6% | 2%-4% |
| Ex 6574 | 8%-10% | 0%-1% | 2%-4% | 4%-6% | 4%-6% |
| Ex 6575 | 8%-10% | 0%-1% | 2%-4% | 4%-6% | 6%-8% |
| Ex 6576 | 8%-10% | 0%-1% | 2%-4% | 4%-6% | 8%-10% |
| Ex 6577 | 8%-10% | 0%-1% | 2%-4% | 6%-8% | 0%-1% |
| Ex 6578 | 8%-10% | 0%-1% | 2%-4% | 6%-8% | 1%-2% |
| Ex 6579 | 8%-10% | 0%-1% | 2%-4% | 6%-8% | 2%-4% |
| Ex 6580 | 8%-10% | 0%-1% | 2%-4% | 6%-8% | 4%-6% |
| Ex 6581 | 8%-10% | 0%-1% | 2%-4% | 6%-8% | 6%-8% |
| Ex 6582 | 8%-10% | 0%-1% | 2%-4% | 6%-8% | 8%-10% |
| Ex 6583 | 8%-10% | 0%-1% | 2%-4% | 8%-10% | 0%-1% |
| Ex 6584 | 8%-10% | 0%-1% | 2%-4% | 8%-10% | 1%-2% |
| Ex 6585 | 8%-10% | 0%-1% | 2%-4% | 8%-10% | 2%-4% |
| Ex 6586 | 8%-10% | 0%-1% | 2%-4% | 8%-10% | 4%-6% |
| Ex 6587 | 8%-10% | 0%-1% | 2%-4% | 8%-10% | 6%-8% |
| Ex 6588 | 8%-10% | 0%-1% | 2%-4% | 8%-10% | 8%-10% |
| Ex 6589 | 8%-10% | 0%-1% | 4%-6% | 0%-1% | 0%-1% |
| Ex 6590 | 8%-10% | 0%-1% | 4%-6% | 0%-1% | 1%-2% |
| Ex 6591 | 8%-10% | 0%-1% | 4%-6% | 0%-1% | 2%-4% |
| Ex 6592 | 8%-10% | 0%-1% | 4%-6% | 0%-1% | 4%-6% |
| Ex 6593 | 8%-10% | 0%-1% | 4%-6% | 0%-1% | 6%-8% |
| Ex 6594 | 8%-10% | 0%-1% | 4%-6% | 0%-1% | 8%-10% |
| Ex 6595 | 8%-10% | 0%-1% | 4%-6% | 1%-2% | 0%-1% |
| Ex 6596 | 8%-10% | 0%-1% | 4%-6% | 1%-2% | 1%-2% |
| Ex 6597 | 8%-10% | 0%-1% | 4%-6% | 1%-2% | 2%-4% |
| Ex 6598 | 8%-10% | 0%-1% | 4%-6% | 1%-2% | 4%-6% |
| Ex 6599 | 8%-10% | 0%-1% | 4%-6% | 1%-2% | 6%-8% |
| Ex 6600 | 8%-10% | 0%-1% | 4%-6% | 1%-2% | 8%-10% |
| Ex 6601 | 8%-10% | 0%-1% | 4%-6% | 2%-4% | 0%-1% |
| Ex 6602 | 8%-10% | 0%-1% | 4%-6% | 2%-4% | 1%-2% |
| Ex 6603 | 8%-10% | 0%-1% | 4%-6% | 2%-4% | 2%-4% |
| Ex 6604 | 8%-10% | 0%-1% | 4%-6% | 2%-4% | 4%-6% |
| Ex 6605 | 8%-10% | 0%-1% | 4%-6% | 2%-4% | 6%-8% |
| Ex 6606 | 8%-10% | 0%-1% | 4%-6% | 2%-4% | 8%-10% |
| Ex 6607 | 8%-10% | 0%-1% | 4%-6% | 4%-6% | 0%-1% |
| Ex 6608 | 8%-10% | 0%-1% | 4%-6% | 4%-6% | 1%-2% |
| Ex 6609 | 8%-10% | 0%-1% | 4%-6% | 4%-6% | 2%-4% |
| Ex 6610 | 8%-10% | 0%-1% | 4%-6% | 4%-6% | 4%-6% |
| Ex 6611 | 8%-10% | 0%-1% | 4%-6% | 4%-6% | 6%-8% |
| Ex 6612 | 8%-10% | 0%-1% | 4%-6% | 4%-6% | 8%-10% |
| Ex 6613 | 8%-10% | 0%-1% | 4%-6% | 6%-8% | 0%-1% |
| Ex 6614 | 8%-10% | 0%-1% | 4%-6% | 6%-8% | 1%-2% |
| Ex 6615 | 8%-10% | 0%-1% | 4%-6% | 6%-8% | 2%-4% |
| Ex 6616 | 8%-10% | 0%-1% | 4%-6% | 6%-8% | 4%-6% |
| Ex 6617 | 8%-10% | 0%-1% | 4%-6% | 6%-8% | 6%-8% |
| Ex 6618 | 8%-10% | 0%-1% | 4%-6% | 6%-8% | 8%-10% |
| Ex 6619 | 8%-10% | 0%-1% | 4%-6% | 8%-10% | 0%-1% |
| Ex 6620 | 8%-10% | 0%-1% | 4%-6% | 8%-10% | 1%-2% |
| Ex 6621 | 8%-10% | 0%-1% | 4%-6% | 8%-10% | 2%-4% |
| Ex 6622 | 8%-10% | 0%-1% | 4%-6% | 8%-10% | 4%-6% |
| Ex 6623 | 8%-10% | 0%-1% | 4%-6% | 8%-10% | 6%-8% |
| Ex 6624 | 8%-10% | 0%-1% | 4%-6% | 8%-10% | 8%-10% |
| Ex 6625 | 8%-10% | 0%-1% | 6%-8% | 0%-1% | 0%-1% |
| Ex 6626 | 8%-10% | 0%-1% | 6%-8% | 0%-1% | 1%-2% |
| Ex 6627 | 8%-10% | 0%-1% | 6%-8% | 0%-1% | 2%-4% |
| Ex 6628 | 8%-10% | 0%-1% | 6%-8% | 0%-1% | 4%-6% |
| Ex 6629 | 8%-10% | 0%-1% | 6%-8% | 0%-1% | 6%-8% |
| Ex 6630 | 8%-10% | 0%-1% | 6%-8% | 0%-1% | 8%-10% |
| Ex 6631 | 8%-10% | 0%-1% | 6%-8% | 1%-2% | 0%-1% |
| Ex 6632 | 8%-10% | 0%-1% | 6%-8% | 1%-2% | 1%-2% |
| Ex 6633 | 8%-10% | 0%-1% | 6%-8% | 1%-2% | 2%-4% |
| Ex 6634 | 8%-10% | 0%-1% | 6%-8% | 1%-2% | 4%-6% |
| Ex 6635 | 8%-10% | 0%-1% | 6%-8% | 1%-2% | 6%-8% |
| Ex 6636 | 8%-10% | 0%-1% | 6%-8% | 1%-2% | 8%-10% |
| Ex 6637 | 8%-10% | 0%-1% | 6%-8% | 2%-4% | 0%-1% |
| Ex 6638 | 8%-10% | 0%-1% | 6%-8% | 2%-4% | 1%-2% |
| Ex 6639 | 8%-10% | 0%-1% | 6%-8% | 2%-4% | 2%-4% |
| Ex 6640 | 8%-10% | 0%-1% | 6%-8% | 2%-4% | 4%-6% |
| Ex 6641 | 8%-10% | 0%-1% | 6%-8% | 2%-4% | 6%-8% |
| Ex 6642 | 8%-10% | 0%-1% | 6%-8% | 2%-4% | 8%-10% |
| Ex 6643 | 8%-10% | 0%-1% | 6%-8% | 4%-6% | 0%-1% |
| Ex 6644 | 8%-10% | 0%-1% | 6%-8% | 4%-6% | 1%-2% |
| Ex 6645 | 8%-10% | 0%-1% | 6%-8% | 4%-6% | 2%-4% |
| Ex 6646 | 8%-10% | 0%-1% | 6%-8% | 4%-6% | 4%-6% |
| Ex 6647 | 8%-10% | 0%-1% | 6%-8% | 4%-6% | 6%-8% |
| Ex 6648 | 8%-10% | 0%-1% | 6%-8% | 4%-6% | 8%-10% |
| Ex 6649 | 8%-10% | 0%-1% | 6%-8% | 6%-8% | 0%-1% |
| Ex 6650 | 8%-10% | 0%-1% | 6%-8% | 6%-8% | 1%-2% |
| Ex 6651 | 8%-10% | 0%-1% | 6%-8% | 6%-8% | 2%-4% |
| Ex 6652 | 8%-10% | 0%-1% | 6%-8% | 6%-8% | 4%-6% |
| Ex 6653 | 8%-10% | 0%-1% | 6%-8% | 6%-8% | 6%-8% |
| Ex 6654 | 8%-10% | 0%-1% | 6%-8% | 6%-8% | 8%-10% |
| Ex 6655 | 8%-10% | 0%-1% | 6%-8% | 8%-10% | 0%-1% |
| Ex 6656 | 8%-10% | 0%-1% | 6%-8% | 8%-10% | 1%-2% |
| Ex 6657 | 8%-10% | 0%-1% | 6%-8% | 8%-10% | 2%-4% |
| Ex 6658 | 8%-10% | 0%-1% | 6%-8% | 8%-10% | 4%-6% |
| Ex 6659 | 8%-10% | 0%-1% | 6%-8% | 8%-10% | 6%-8% |
| Ex 6660 | 8%-10% | 0%-1% | 6%-8% | 8%-10% | 8%-10% |
| Ex 6661 | 8%-10% | 0%-1% | 8%-10% | 0%-1% | 0%-1% |
| Ex 6662 | 8%-10% | 0%-1% | 8%-10% | 0%-1% | 1%-2% |
| Ex 6663 | 8%-10% | 0%-1% | 8%-10% | 0%-1% | 2%-4% |
| Ex 6664 | 8%-10% | 0%-1% | 8%-10% | 0%-1% | 4%-6% |
| Ex 6665 | 8%-10% | 0%-1% | 8%-10% | 0%-1% | 6%-8% |
| Ex 6666 | 8%-10% | 0%-1% | 8%-10% | 0%-1% | 8%-10% |
| Ex 6667 | 8%-10% | 0%-1% | 8%-10% | 1%-2% | 0%-1% |
| Ex 6668 | 8%-10% | 0%-1% | 8%-10% | 1%-2% | 1%-2% |
| Ex 6669 | 8%-10% | 0%-1% | 8%-10% | 1%-2% | 2%-4% |
| Ex 6670 | 8%-10% | 0%-1% | 8%-10% | 1%-2% | 4%-6% |
| Ex 6671 | 8%-10% | 0%-1% | 8%-10% | 1%-2% | 6%-8% |
| Ex 6672 | 8%-10% | 0%-1% | 8%-10% | 1%-2% | 8%-10% |
| Ex 6673 | 8%-10% | 0%-1% | 8%-10% | 2%-4% | 0%-1% |
| Ex 6674 | 8%-10% | 0%-1% | 8%-10% | 2%-4% | 1%-2% |
| Ex 6675 | 8%-10% | 0%-1% | 8%-10% | 2%-4% | 2%-4% |
| Ex 6676 | 8%-10% | 0%-1% | 8%-10% | 2%-4% | 4%-6% |
| Ex 6677 | 8%-10% | 0%-1% | 8%-10% | 2%-4% | 6%-8% |
| Ex 6678 | 8%-10% | 0%-1% | 8%-10% | 2%-4% | 8%-10% |
| Ex 6679 | 8%-10% | 0%-1% | 8%-10% | 4%-6% | 0%-1% |
| Ex 6680 | 8%-10% | 0%-1% | 8%-10% | 4%-6% | 1%-2% |
| Ex 6681 | 8%-10% | 0%-1% | 8%-10% | 4%-6% | 2%-4% |
| Ex 6682 | 8%-10% | 0%-1% | 8%-10% | 4%-6% | 4%-6% |
| Ex 6683 | 8%-10% | 0%-1% | 8%-10% | 4%-6% | 6%-8% |
| Ex 6684 | 8%-10% | 0%-1% | 8%-10% | 4%-6% | 8%-10% |
| Ex 6685 | 8%-10% | 0%-1% | 8%-10% | 6%-8% | 0%-1% |
| Ex 6686 | 8%-10% | 0%-1% | 8%-10% | 6%-8% | 1%-2% |
| Ex 6687 | 8%-10% | 0%-1% | 8%-10% | 6%-8% | 2%-4% |
| Ex 6688 | 8%-10% | 0%-1% | 8%-10% | 6%-8% | 4%-6% |
| Ex 6689 | 8%-10% | 0%-1% | 8%-10% | 6%-8% | 6%-8% |
| Ex 6690 | 8%-10% | 0%-1% | 8%-10% | 6%-8% | 8%-10% |
| Ex 6691 | 8%-10% | 0%-1% | 8%-10% | 8%-10% | 0%-1% |
| Ex 6692 | 8%-10% | 0%-1% | 8%-10% | 8%-10% | 1%-2% |
| Ex 6693 | 8%-10% | 0%-1% | 8%-10% | 8%-10% | 2%-4% |
| Ex 6694 | 8%-10% | 0%-1% | 8%-10% | 8%-10% | 4%-6% |
| Ex 6695 | 8%-10% | 0%-1% | 8%-10% | 8%-10% | 6%-8% |
| Ex 6696 | 8%-10% | 0%-1% | 8%-10% | 8%-10% | 8%-10% |
| Ex 6697 | 8%-10% | 1%-2% | 0%-1% | 0%-1% | 0%-1% |
| Ex 6698 | 8%-10% | 1%-2% | 0%-1% | 0%-1% | 1%-2% |
| Ex 6699 | 8%-10% | 1%-2% | 0%-1% | 0%-1% | 2%-4% |
| Ex 6700 | 8%-10% | 1%-2% | 0%-1% | 0%-1% | 4%-6% |
| Ex 6701 | 8%-10% | 1%-2% | 0%-1% | 0%-1% | 6%-8% |
| Ex 6702 | 8%-10% | 1%-2% | 0%-1% | 0%-1% | 8%-10% |
| Ex 6703 | 8%-10% | 1%-2% | 0%-1% | 1%-2% | 0%-1% |
| Ex 6704 | 8%-10% | 1%-2% | 0%-1% | 1%-2% | 1%-2% |
| Ex 6705 | 8%-10% | 1%-2% | 0%-1% | 1%-2% | 2%-4% |
| Ex 6706 | 8%-10% | 1%-2% | 0%-1% | 1%-2% | 4%-6% |
| Ex 6707 | 8%-10% | 1%-2% | 0%-1% | 1%-2% | 6%-8% |
| Ex 6708 | 8%-10% | 1%-2% | 0%-1% | 1%-2% | 8%-10% |
| Ex 6709 | 8%-10% | 1%-2% | 0%-1% | 2%-4% | 0%-1% |
| Ex 6710 | 8%-10% | 1%-2% | 0%-1% | 2%-4% | 1%-2% |
| Ex 6711 | 8%-10% | 1%-2% | 0%-1% | 2%-4% | 2%-4% |
| Ex 6712 | 8%-10% | 1%-2% | 0%-1% | 2%-4% | 4%-6% |
| Ex 6713 | 8%-10% | 1%-2% | 0%-1% | 2%-4% | 6%-8% |
| Ex 6714 | 8%-10% | 1%-2% | 0%-1% | 2%-4% | 8%-10% |
| Ex 6715 | 8%-10% | 1%-2% | 0%-1% | 4%-6% | 0%-1% |
| Ex 6716 | 8%-10% | 1%-2% | 0%-1% | 4%-6% | 1%-2% |
| Ex 6717 | 8%-10% | 1%-2% | 0%-1% | 4%-6% | 2%-4% |
| Ex 6718 | 8%-10% | 1%-2% | 0%-1% | 4%-6% | 4%-6% |
| Ex 6719 | 8%-10% | 1%-2% | 0%-1% | 4%-6% | 6%-8% |
| Ex 6720 | 8%-10% | 1%-2% | 0%-1% | 4%-6% | 8%-10% |
| Ex 6721 | 8%-10% | 1%-2% | 0%-1% | 6%-8% | 0%-1% |
| Ex 6722 | 8%-10% | 1%-2% | 0%-1% | 6%-8% | 1%-2% |
| Ex 6723 | 8%-10% | 1%-2% | 0%-1% | 6%-8% | 2%-4% |
| Ex 6724 | 8%-10% | 1%-2% | 0%-1% | 6%-8% | 4%-6% |
| Ex 6725 | 8%-10% | 1%-2% | 0%-1% | 6%-8% | 6%-8% |
| Ex 6726 | 8%-10% | 1%-2% | 0%-1% | 6%-8% | 8%-10% |
| Ex 6727 | 8%-10% | 1%-2% | 0%-1% | 8%-10% | 0%-1% |
| Ex 6728 | 8%-10% | 1%-2% | 0%-1% | 8%-10% | 1%-2% |
| Ex 6729 | 8%-10% | 1%-2% | 0%-1% | 8%-10% | 2%-4% |
| Ex 6730 | 8%-10% | 1%-2% | 0%-1% | 8%-10% | 4%-6% |
| Ex 6731 | 8%-10% | 1%-2% | 0%-1% | 8%-10% | 6%-8% |
| Ex 6732 | 8%-10% | 1%-2% | 0%-1% | 8%-10% | 8%-10% |
| Ex 6733 | 8%-10% | 1%-2% | 1%-2% | 0%-1% | 0%-1% |
| Ex 6734 | 8%-10% | 1%-2% | 1%-2% | 0%-1% | 1%-2% |
| Ex 6735 | 8%-10% | 1%-2% | 1%-2% | 0%-1% | 2%-4% |
| Ex 6736 | 8%-10% | 1%-2% | 1%-2% | 0%-1% | 4%-6% |
| Ex 6737 | 8%-10% | 1%-2% | 1%-2% | 0%-1% | 6%-8% |
| Ex 6738 | 8%-10% | 1%-2% | 1%-2% | 0%-1% | 8%-10% |
| Ex 6739 | 8%-10% | 1%-2% | 1%-2% | 1%-2% | 0%-1% |
| Ex 6740 | 8%-10% | 1%-2% | 1%-2% | 1%-2% | 1%-2% |
| Ex 6741 | 8%-10% | 1%-2% | 1%-2% | 1%-2% | 2%-4% |
| Ex 6742 | 8%-10% | 1%-2% | 1%-2% | 1%-2% | 4%-6% |
| Ex 6743 | 8%-10% | 1%-2% | 1%-2% | 1%-2% | 6%-8% |
| Ex 6744 | 8%-10% | 1%-2% | 1%-2% | 1%-2% | 8%-10% |
| Ex 6745 | 8%-10% | 1%-2% | 1%-2% | 2%-4% | 0%-1% |
| Ex 6746 | 8%-10% | 1%-2% | 1%-2% | 2%-4% | 1%-2% |
| Ex 6747 | 8%-10% | 1%-2% | 1%-2% | 2%-4% | 2%-4% |
| Ex 6748 | 8%-10% | 1%-2% | 1%-2% | 2%-4% | 4%-6% |
| Ex 6749 | 8%-10% | 1%-2% | 1%-2% | 2%-4% | 6%-8% |
| Ex 6750 | 8%-10% | 1%-2% | 1%-2% | 2%-4% | 8%-10% |
| Ex 6751 | 8%-10% | 1%-2% | 1%-2% | 4%-6% | 0%-1% |
| Ex 6752 | 8%-10% | 1%-2% | 1%-2% | 4%-6% | 1%-2% |
| Ex 6753 | 8%-10% | 1%-2% | 1%-2% | 4%-6% | 2%-4% |
| Ex 6754 | 8%-10% | 1%-2% | 1%-2% | 4%-6% | 4%-6% |
| Ex 6755 | 8%-10% | 1%-2% | 1%-2% | 4%-6% | 6%-8% |
| Ex 6756 | 8%-10% | 1%-2% | 1%-2% | 4%-6% | 8%-10% |
| Ex 6757 | 8%-10% | 1%-2% | 1%-2% | 6%-8% | 0%-1% |
| Ex 6758 | 8%-10% | 1%-2% | 1%-2% | 6%-8% | 1%-2% |
| Ex 6759 | 8%-10% | 1%-2% | 1%-2% | 6%-8% | 2%-4% |
| Ex 6760 | 8%-10% | 1%-2% | 1%-2% | 6%-8% | 4%-6% |
| Ex 6761 | 8%-10% | 1%-2% | 1%-2% | 6%-8% | 6%-8% |
| Ex 6762 | 8%-10% | 1%-2% | 1%-2% | 6%-8% | 8%-10% |
| Ex 6763 | 8%-10% | 1%-2% | 1%-2% | 8%-10% | 0%-1% |
| Ex 6764 | 8%-10% | 1%-2% | 1%-2% | 8%-10% | 1%-2% |
| Ex 6765 | 8%-10% | 1%-2% | 1%-2% | 8%-10% | 2%-4% |
| Ex 6766 | 8%-10% | 1%-2% | 1%-2% | 8%-10% | 4%-6% |
| Ex 6767 | 8%-10% | 1%-2% | 1%-2% | 8%-10% | 6%-8% |
| Ex 6768 | 8%-10% | 1%-2% | 1%-2% | 8%-10% | 8%-10% |
| Ex 6769 | 8%-10% | 1%-2% | 2%-4% | 0%-1% | 0%-1% |
| Ex 6770 | 8%-10% | 1%-2% | 2%-4% | 0%-1% | 1%-2% |
| Ex 6771 | 8%-10% | 1%-2% | 2%-4% | 0%-1% | 2%-4% |
| Ex 6772 | 8%-10% | 1%-2% | 2%-4% | 0%-1% | 4%-6% |
| Ex 6773 | 8%-10% | 1%-2% | 2%-4% | 0%-1% | 6%-8% |
| Ex 6774 | 8%-10% | 1%-2% | 2%-4% | 0%-1% | 8%-10% |
| Ex 6775 | 8%-10% | 1%-2% | 2%-4% | 1%-2% | 0%-1% |
| Ex 6776 | 8%-10% | 1%-2% | 2%-4% | 1%-2% | 1%-2% |
| Ex 6777 | 8%-10% | 1%-2% | 2%-4% | 1%-2% | 2%-4% |
| Ex 6778 | 8%-10% | 1%-2% | 2%-4% | 1%-2% | 4%-6% |
| Ex 6779 | 8%-10% | 1%-2% | 2%-4% | 1%-2% | 6%-8% |
| Ex 6780 | 8%-10% | 1%-2% | 2%-4% | 1%-2% | 8%-10% |
| Ex 6781 | 8%-10% | 1%-2% | 2%-4% | 2%-4% | 0%-1% |
| Ex 6782 | 8%-10% | 1%-2% | 2%-4% | 2%-4% | 1%-2% |
| Ex 6783 | 8%-10% | 1%-2% | 2%-4% | 2%-4% | 2%-4% |
| Ex 6784 | 8%-10% | 1%-2% | 2%-4% | 2%-4% | 4%-6% |
| Ex 6785 | 8%-10% | 1%-2% | 2%-4% | 2%-4% | 6%-8% |
| Ex 6786 | 8%-10% | 1%-2% | 2%-4% | 2%-4% | 8%-10% |
| Ex 6787 | 8%-10% | 1%-2% | 2%-4% | 4%-6% | 0%-1% |
| Ex 6788 | 8%-10% | 1%-2% | 2%-4% | 4%-6% | 1%-2% |
| Ex 6789 | 8%-10% | 1%-2% | 2%-4% | 4%-6% | 2%-4% |
| Ex 6790 | 8%-10% | 1%-2% | 2%-4% | 4%-6% | 4%-6% |
| Ex 6791 | 8%-10% | 1%-2% | 2%-4% | 4%-6% | 6%-8% |
| Ex 6792 | 8%-10% | 1%-2% | 2%-4% | 4%-6% | 8%-10% |
| Ex 6793 | 8%-10% | 1%-2% | 2%-4% | 6%-8% | 0%-1% |
| Ex 6794 | 8%-10% | 1%-2% | 2%-4% | 6%-8% | 1%-2% |
| Ex 6795 | 8%-10% | 1%-2% | 2%-4% | 6%-8% | 2%-4% |
| Ex 6796 | 8%-10% | 1%-2% | 2%-4% | 6%-8% | 4%-6% |
| Ex 6797 | 8%-10% | 1%-2% | 2%-4% | 6%-8% | 6%-8% |
| Ex 6798 | 8%-10% | 1%-2% | 2%-4% | 6%-8% | 8%-10% |
| Ex 6799 | 8%-10% | 1%-2% | 2%-4% | 8%-10% | 0%-1% |
| Ex 6800 | 8%-10% | 1%-2% | 2%-4% | 8%-10% | 1%-2% |
| Ex 6801 | 8%-10% | 1%-2% | 2%-4% | 8%-10% | 2%-4% |
| Ex 6802 | 8%-10% | 1%-2% | 2%-4% | 8%-10% | 4%-6% |
| Ex 6803 | 8%-10% | 1%-2% | 2%-4% | 8%-10% | 6%-8% |
| Ex 6804 | 8%-10% | 1%-2% | 2%-4% | 8%-10% | 8%-10% |
| Ex 6805 | 8%-10% | 1%-2% | 4%-6% | 0%-1% | 0%-1% |
| Ex 6806 | 8%-10% | 1%-2% | 4%-6% | 0%-1% | 1%-2% |
| Ex 6807 | 8%-10% | 1%-2% | 4%-6% | 0%-1% | 2%-4% |
| Ex 6808 | 8%-10% | 1%-2% | 4%-6% | 0%-1% | 4%-6% |
| Ex 6809 | 8%-10% | 1%-2% | 4%-6% | 0%-1% | 6%-8% |
| Ex 6810 | 8%-10% | 1%-2% | 4%-6% | 0%-1% | 8%-10% |
| Ex 6811 | 8%-10% | 1%-2% | 4%-6% | 1%-2% | 0%-1% |
| Ex 6812 | 8%-10% | 1%-2% | 4%-6% | 1%-2% | 1%-2% |
| Ex 6813 | 8%-10% | 1%-2% | 4%-6% | 1%-2% | 2%-4% |
| Ex 6814 | 8%-10% | 1%-2% | 4%-6% | 1%-2% | 4%-6% |
| Ex 6815 | 8%-10% | 1%-2% | 4%-6% | 1%-2% | 6%-8% |
| Ex 6816 | 8%-10% | 1%-2% | 4%-6% | 1%-2% | 8%-10% |
| Ex 6817 | 8%-10% | 1%-2% | 4%-6% | 2%-4% | 0%-1% |
| Ex 6818 | 8%-10% | 1%-2% | 4%-6% | 2%-4% | 1%-2% |
| Ex 6819 | 8%-10% | 1%-2% | 4%-6% | 2%-4% | 2%-4% |
| Ex 6820 | 8%-10% | 1%-2% | 4%-6% | 2%-4% | 4%-6% |
| Ex 6821 | 8%-10% | 1%-2% | 4%-6% | 2%-4% | 6%-8% |
| Ex 6822 | 8%-10% | 1%-2% | 4%-6% | 2%-4% | 8%-10% |
| Ex 6823 | 8%-10% | 1%-2% | 4%-6% | 4%-6% | 0%-1% |
| Ex 6824 | 8%-10% | 1%-2% | 4%-6% | 4%-6% | 1%-2% |
| Ex 6825 | 8%-10% | 1%-2% | 4%-6% | 4%-6% | 2%-4% |
| Ex 6826 | 8%-10% | 1%-2% | 4%-6% | 4%-6% | 4%-6% |
| Ex 6827 | 8%-10% | 1%-2% | 4%-6% | 4%-6% | 6%-8% |
| Ex 6828 | 8%-10% | 1%-2% | 4%-6% | 4%-6% | 8%-10% |
| Ex 6829 | 8%-10% | 1%-2% | 4%-6% | 6%-8% | 0%-1% |
| Ex 6830 | 8%-10% | 1%-2% | 4%-6% | 6%-8% | 1%-2% |
| Ex 6831 | 8%-10% | 1%-2% | 4%-6% | 6%-8% | 2%-4% |
| Ex 6832 | 8%-10% | 1%-2% | 4%-6% | 6%-8% | 4%-6% |
| Ex 6833 | 8%-10% | 1%-2% | 4%-6% | 6%-8% | 6%-8% |
| Ex 6834 | 8%-10% | 1%-2% | 4%-6% | 6%-8% | 8%-10% |
| Ex 6835 | 8%-10% | 1%-2% | 4%-6% | 8%-10% | 0%-1% |
| Ex 6836 | 8%-10% | 1%-2% | 4%-6% | 8%-10% | 1%-2% |
| Ex 6837 | 8%-10% | 1%-2% | 4%-6% | 8%-10% | 2%-4% |
| Ex 6838 | 8%-10% | 1%-2% | 4%-6% | 8%-10% | 4%-6% |
| Ex 6839 | 8%-10% | 1%-2% | 4%-6% | 8%-10% | 6%-8% |
| Ex 6840 | 8%-10% | 1%-2% | 4%-6% | 8%-10% | 8%-10% |
| Ex 6841 | 8%-10% | 1%-2% | 6%-8% | 0%-1% | 0%-1% |
| Ex 6842 | 8%-10% | 1%-2% | 6%-8% | 0%-1% | 1%-2% |
| Ex 6843 | 8%-10% | 1%-2% | 6%-8% | 0%-1% | 2%-4% |
| Ex 6844 | 8%-10% | 1%-2% | 6%-8% | 0%-1% | 4%-6% |
| Ex 6845 | 8%-10% | 1%-2% | 6%-8% | 0%-1% | 6%-8% |
| Ex 6846 | 8%-10% | 1%-2% | 6%-8% | 0%-1% | 8%-10% |
| Ex 6847 | 8%-10% | 1%-2% | 6%-8% | 1%-2% | 0%-1% |
| Ex 6848 | 8%-10% | 1%-2% | 6%-8% | 1%-2% | 1%-2% |
| Ex 6849 | 8%-10% | 1%-2% | 6%-8% | 1%-2% | 2%-4% |
| Ex 6850 | 8%-10% | 1%-2% | 6%-8% | 1%-2% | 4%-6% |
| Ex 6851 | 8%-10% | 1%-2% | 6%-8% | 1%-2% | 6%-8% |
| Ex 6852 | 8%-10% | 1%-2% | 6%-8% | 1%-2% | 8%-10% |
| Ex 6853 | 8%-10% | 1%-2% | 6%-8% | 2%-4% | 0%-1% |
| Ex 6854 | 8%-10% | 1%-2% | 6%-8% | 2%-4% | 1%-2% |
| Ex 6855 | 8%-10% | 1%-2% | 6%-8% | 2%-4% | 2%-4% |
| Ex 6856 | 8%-10% | 1%-2% | 6%-8% | 2%-4% | 4%-6% |
| Ex 6857 | 8%-10% | 1%-2% | 6%-8% | 2%-4% | 6%-8% |
| Ex 6858 | 8%-10% | 1%-2% | 6%-8% | 2%-4% | 8%-10% |
| Ex 6859 | 8%-10% | 1%-2% | 6%-8% | 4%-6% | 0%-1% |
| Ex 6860 | 8%-10% | 1%-2% | 6%-8% | 4%-6% | 1%-2% |
| Ex 6861 | 8%-10% | 1%-2% | 6%-8% | 4%-6% | 2%-4% |
| Ex 6862 | 8%-10% | 1%-2% | 6%-8% | 4%-6% | 4%-6% |
| Ex 6863 | 8%-10% | 1%-2% | 6%-8% | 4%-6% | 6%-8% |
| Ex 6864 | 8%-10% | 1%-2% | 6%-8% | 4%-6% | 8%-10% |
| Ex 6865 | 8%-10% | 1%-2% | 6%-8% | 6%-8% | 0%-1% |
| Ex 6866 | 8%-10% | 1%-2% | 6%-8% | 6%-8% | 1%-2% |
| Ex 6867 | 8%-10% | 1%-2% | 6%-8% | 6%-8% | 2%-4% |
| Ex 6868 | 8%-10% | 1%-2% | 6%-8% | 6%-8% | 4%-6% |
| Ex 6869 | 8%-10% | 1%-2% | 6%-8% | 6%-8% | 6%-8% |
| Ex 6870 | 8%-10% | 1%-2% | 6%-8% | 6%-8% | 8%-10% |
| Ex 6871 | 8%-10% | 1%-2% | 6%-8% | 8%-10% | 0%-1% |
| Ex 6872 | 8%-10% | 1%-2% | 6%-8% | 8%-10% | 1%-2% |
| Ex 6873 | 8%-10% | 1%-2% | 6%-8% | 8%-10% | 2%-4% |
| Ex 6874 | 8%-10% | 1%-2% | 6%-8% | 8%-10% | 4%-6% |
| Ex 6875 | 8%-10% | 1%-2% | 6%-8% | 8%-10% | 6%-8% |
| Ex 6876 | 8%-10% | 1%-2% | 6%-8% | 8%-10% | 8%-10% |
| Ex 6877 | 8%-10% | 1%-2% | 8%-10% | 0%-1% | 0%-1% |
| Ex 6878 | 8%-10% | 1%-2% | 8%-10% | 0%-1% | 1%-2% |
| Ex 6879 | 8%-10% | 1%-2% | 8%-10% | 0%-1% | 2%-4% |
| Ex 6880 | 8%-10% | 1%-2% | 8%-10% | 0%-1% | 4%-6% |
| Ex 6881 | 8%-10% | 1%-2% | 8%-10% | 0%-1% | 6%-8% |
| Ex 6882 | 8%-10% | 1%-2% | 8%-10% | 0%-1% | 8%-10% |
| Ex 6883 | 8%-10% | 1%-2% | 8%-10% | 1%-2% | 0%-1% |
| Ex 6884 | 8%-10% | 1%-2% | 8%-10% | 1%-2% | 1%-2% |
| Ex 6885 | 8%-10% | 1%-2% | 8%-10% | 1%-2% | 2%-4% |
| Ex 6886 | 8%-10% | 1%-2% | 8%-10% | 1%-2% | 4%-6% |
| Ex 6887 | 8%-10% | 1%-2% | 8%-10% | 1%-2% | 6%-8% |
| Ex 6888 | 8%-10% | 1%-2% | 8%-10% | 1%-2% | 8%-10% |
| Ex 6889 | 8%-10% | 1%-2% | 8%-10% | 2%-4% | 0%-1% |
| Ex 6890 | 8%-10% | 1%-2% | 8%-10% | 2%-4% | 1%-2% |
| Ex 6891 | 8%-10% | 1%-2% | 8%-10% | 2%-4% | 2%-4% |
| Ex 6892 | 8%-10% | 1%-2% | 8%-10% | 2%-4% | 4%-6% |
| Ex 6893 | 8%-10% | 1%-2% | 8%-10% | 2%-4% | 6%-8% |
| Ex 6894 | 8%-10% | 1%-2% | 8%-10% | 2%-4% | 8%-10% |
| Ex 6895 | 8%-10% | 1%-2% | 8%-10% | 4%-6% | 0%-1% |
| Ex 6896 | 8%-10% | 1%-2% | 8%-10% | 4%-6% | 1%-2% |
| Ex 6897 | 8%-10% | 1%-2% | 8%-10% | 4%-6% | 2%-4% |
| Ex 6898 | 8%-10% | 1%-2% | 8%-10% | 4%-6% | 4%-6% |
| Ex 6899 | 8%-10% | 1%-2% | 8%-10% | 4%-6% | 6%-8% |
| Ex 6900 | 8%-10% | 1%-2% | 8%-10% | 4%-6% | 8%-10% |
| Ex 6901 | 8%-10% | 1%-2% | 8%-10% | 6%-8% | 0%-1% |
| Ex 6902 | 8%-10% | 1%-2% | 8%-10% | 6%-8% | 1%-2% |
| Ex 6903 | 8%-10% | 1%-2% | 8%-10% | 6%-8% | 2%-4% |
| Ex 6904 | 8%-10% | 1%-2% | 8%-10% | 6%-8% | 4%-6% |
| Ex 6905 | 8%-10% | 1%-2% | 8%-10% | 6%-8% | 6%-8% |
| Ex 6906 | 8%-10% | 1%-2% | 8%-10% | 6%-8% | 8%-10% |
| Ex 6907 | 8%-10% | 1%-2% | 8%-10% | 8%-10% | 0%-1% |
| Ex 6908 | 8%-10% | 1%-2% | 8%-10% | 8%-10% | 1%-2% |
| Ex 6909 | 8%-10% | 1%-2% | 8%-10% | 8%-10% | 2%-4% |
| Ex 6910 | 8%-10% | 1%-2% | 8%-10% | 8%-10% | 4%-6% |
| Ex 6911 | 8%-10% | 1%-2% | 8%-10% | 8%-10% | 6%-8% |
| Ex 6912 | 8%-10% | 1%-2% | 8%-10% | 8%-10% | 8%-10% |
| Ex 6913 | 8%-10% | 2%-4% | 0%-1% | 0%-1% | 0%-1% |
| Ex 6914 | 8%-10% | 2%-4% | 0%-1% | 0%-1% | 1%-2% |
| Ex 6915 | 8%-10% | 2%-4% | 0%-1% | 0%-1% | 2%-4% |
| Ex 6916 | 8%-10% | 2%-4% | 0%-1% | 0%-1% | 4%-6% |
| Ex 6917 | 8%-10% | 2%-4% | 0%-1% | 0%-1% | 6%-8% |
| Ex 6918 | 8%-10% | 2%-4% | 0%-1% | 0%-1% | 8%-10% |
| Ex 6919 | 8%-10% | 2%-4% | 0%-1% | 1%-2% | 0%-1% |
| Ex 6920 | 8%-10% | 2%-4% | 0%-1% | 1%-2% | 1%-2% |
| Ex 6921 | 8%-10% | 2%-4% | 0%-1% | 1%-2% | 2%-4% |
| Ex 6922 | 8%-10% | 2%-4% | 0%-1% | 1%-2% | 4%-6% |
| Ex 6923 | 8%-10% | 2%-4% | 0%-1% | 1%-2% | 6%-8% |
| Ex 6924 | 8%-10% | 2%-4% | 0%-1% | 1%-2% | 8%-10% |
| Ex 6925 | 8%-10% | 2%-4% | 0%-1% | 2%-4% | 0%-1% |
| Ex 6926 | 8%-10% | 2%-4% | 0%-1% | 2%-4% | 1%-2% |
| Ex 6927 | 8%-10% | 2%-4% | 0%-1% | 2%-4% | 2%-4% |
| Ex 6928 | 8%-10% | 2%-4% | 0%-1% | 2%-4% | 4%-6% |
| Ex 6929 | 8%-10% | 2%-4% | 0%-1% | 2%-4% | 6%-8% |
| Ex 6930 | 8%-10% | 2%-4% | 0%-1% | 2%-4% | 8%-10% |
| Ex 6931 | 8%-10% | 2%-4% | 0%-1% | 4%-6% | 0%-1% |
| Ex 6932 | 8%-10% | 2%-4% | 0%-1% | 4%-6% | 1%-2% |
| Ex 6933 | 8%-10% | 2%-4% | 0%-1% | 4%-6% | 2%-4% |
| Ex 6934 | 8%-10% | 2%-4% | 0%-1% | 4%-6% | 4%-6% |
| Ex 6935 | 8%-10% | 2%-4% | 0%-1% | 4%-6% | 6%-8% |
| Ex 6936 | 8%-10% | 2%-4% | 0%-1% | 4%-6% | 8%-10% |
| Ex 6937 | 8%-10% | 2%-4% | 0%-1% | 6%-8% | 0%-1% |
| Ex 6938 | 8%-10% | 2%-4% | 0%-1% | 6%-8% | 1%-2% |
| Ex 6939 | 8%-10% | 2%-4% | 0%-1% | 6%-8% | 2%-4% |
| Ex 6940 | 8%-10% | 2%-4% | 0%-1% | 6%-8% | 4%-6% |
| Ex 6941 | 8%-10% | 2%-4% | 0%-1% | 6%-8% | 6%-8% |
| Ex 6942 | 8%-10% | 2%-4% | 0%-1% | 6%-8% | 8%-10% |
| Ex 6943 | 8%-10% | 2%-4% | 0%-1% | 8%-10% | 0%-1% |
| Ex 6944 | 8%-10% | 2%-4% | 0%-1% | 8%-10% | 1%-2% |
| Ex 6945 | 8%-10% | 2%-4% | 0%-1% | 8%-10% | 2%-4% |
| Ex 6946 | 8%-10% | 2%-4% | 0%-1% | 8%-10% | 4%-6% |
| Ex 6947 | 8%-10% | 2%-4% | 0%-1% | 8%-10% | 6%-8% |
| Ex 6948 | 8%-10% | 2%-4% | 0%-1% | 8%-10% | 8%-10% |
| Ex 6949 | 8%-10% | 2%-4% | 1%-2% | 0%-1% | 0%-1% |
| Ex 6950 | 8%-10% | 2%-4% | 1%-2% | 0%-1% | 1%-2% |
| Ex 6951 | 8%-10% | 2%-4% | 1%-2% | 0%-1% | 2%-4% |
| Ex 6952 | 8%-10% | 2%-4% | 1%-2% | 0%-1% | 4%-6% |
| Ex 6953 | 8%-10% | 2%-4% | 1%-2% | 0%-1% | 6%-8% |
| Ex 6954 | 8%-10% | 2%-4% | 1%-2% | 0%-1% | 8%-10% |
| Ex 6955 | 8%-10% | 2%-4% | 1%-2% | 1%-2% | 0%-1% |
| Ex 6956 | 8%-10% | 2%-4% | 1%-2% | 1%-2% | 1%-2% |
| Ex 6957 | 8%-10% | 2%-4% | 1%-2% | 1%-2% | 2%-4% |
| Ex 6958 | 8%-10% | 2%-4% | 1%-2% | 1%-2% | 4%-6% |
| Ex 6959 | 8%-10% | 2%-4% | 1%-2% | 1%-2% | 6%-8% |
| Ex 6960 | 8%-10% | 2%-4% | 1%-2% | 1%-2% | 8%-10% |
| Ex 6961 | 8%-10% | 2%-4% | 1%-2% | 2%-4% | 0%-1% |
| Ex 6962 | 8%-10% | 2%-4% | 1%-2% | 2%-4% | 1%-2% |
| Ex 6963 | 8%-10% | 2%-4% | 1%-2% | 2%-4% | 2%-4% |
| Ex 6964 | 8%-10% | 2%-4% | 1%-2% | 2%-4% | 4%-6% |
| Ex 6965 | 8%-10% | 2%-4% | 1%-2% | 2%-4% | 6%-8% |
| Ex 6966 | 8%-10% | 2%-4% | 1%-2% | 2%-4% | 8%-10% |
| Ex 6967 | 8%-10% | 2%-4% | 1%-2% | 4%-6% | 0%-1% |
| Ex 6968 | 8%-10% | 2%-4% | 1%-2% | 4%-6% | 1%-2% |
| Ex 6969 | 8%-10% | 2%-4% | 1%-2% | 4%-6% | 2%-4% |
| Ex 6970 | 8%-10% | 2%-4% | 1%-2% | 4%-6% | 4%-6% |
| Ex 6971 | 8%-10% | 2%-4% | 1%-2% | 4%-6% | 6%-8% |
| Ex 6972 | 8%-10% | 2%-4% | 1%-2% | 4%-6% | 8%-10% |
| Ex 6973 | 8%-10% | 2%-4% | 1%-2% | 6%-8% | 0%-1% |
| Ex 6974 | 8%-10% | 2%-4% | 1%-2% | 6%-8% | 1%-2% |
| Ex 6975 | 8%-10% | 2%-4% | 1%-2% | 6%-8% | 2%-4% |
| Ex 6976 | 8%-10% | 2%-4% | 1%-2% | 6%-8% | 4%-6% |
| Ex 6977 | 8%-10% | 2%-4% | 1%-2% | 6%-8% | 6%-8% |
| Ex 6978 | 8%-10% | 2%-4% | 1%-2% | 6%-8% | 8%-10% |
| Ex 6979 | 8%-10% | 2%-4% | 1%-2% | 8%-10% | 0%-1% |
| Ex 6980 | 8%-10% | 2%-4% | 1%-2% | 8%-10% | 1%-2% |
| Ex 6981 | 8%-10% | 2%-4% | 1%-2% | 8%-10% | 2%-4% |
| Ex 6982 | 8%-10% | 2%-4% | 1%-2% | 8%-10% | 4%-6% |
| Ex 6983 | 8%-10% | 2%-4% | 1%-2% | 8%-10% | 6%-8% |
| Ex 6984 | 8%-10% | 2%-4% | 1%-2% | 8%-10% | 8%-10% |
| Ex 6985 | 8%-10% | 2%-4% | 2%-4% | 0%-1% | 0%-1% |
| Ex 6986 | 8%-10% | 2%-4% | 2%-4% | 0%-1% | 1%-2% |
| Ex 6987 | 8%-10% | 2%-4% | 2%-4% | 0%-1% | 2%-4% |
| Ex 6988 | 8%-10% | 2%-4% | 2%-4% | 0%-1% | 4%-6% |
| Ex 6989 | 8%-10% | 2%-4% | 2%-4% | 0%-1% | 6%-8% |
| Ex 6990 | 8%-10% | 2%-4% | 2%-4% | 0%-1% | 8%-10% |
| Ex 6991 | 8%-10% | 2%-4% | 2%-4% | 1%-2% | 0%-1% |
| Ex 6992 | 8%-10% | 2%-4% | 2%-4% | 1%-2% | 1%-2% |
| Ex 6993 | 8%-10% | 2%-4% | 2%-4% | 1%-2% | 2%-4% |
| Ex 6994 | 8%-10% | 2%-4% | 2%-4% | 1%-2% | 4%-6% |
| Ex 6995 | 8%-10% | 2%-4% | 2%-4% | 1%-2% | 6%-8% |
| Ex 6996 | 8%-10% | 2%-4% | 2%-4% | 1%-2% | 8%-10% |
| Ex 6997 | 8%-10% | 2%-4% | 2%-4% | 2%-4% | 0%-1% |
| Ex 6998 | 8%-10% | 2%-4% | 2%-4% | 2%-4% | 1%-2% |
| Ex 6999 | 8%-10% | 2%-4% | 2%-4% | 2%-4% | 2%-4% |
| Ex 7000 | 8%-10% | 2%-4% | 2%-4% | 2%-4% | 4%-6% |
| Ex 7001 | 8%-10% | 2%-4% | 2%-4% | 2%-4% | 6%-8% |
| Ex 7002 | 8%-10% | 2%-4% | 2%-4% | 2%-4% | 8%-10% |
| Ex 7003 | 8%-10% | 2%-4% | 2%-4% | 4%-6% | 0%-1% |
| Ex 7004 | 8%-10% | 2%-4% | 2%-4% | 4%-6% | 1%-2% |
| Ex 7005 | 8%-10% | 2%-4% | 2%-4% | 4%-6% | 2%-4% |
| Ex 7006 | 8%-10% | 2%-4% | 2%-4% | 4%-6% | 4%-6% |
| Ex 7007 | 8%-10% | 2%-4% | 2%-4% | 4%-6% | 6%-8% |
| Ex 7008 | 8%-10% | 2%-4% | 2%-4% | 4%-6% | 8%-10% |
| Ex 7009 | 8%-10% | 2%-4% | 2%-4% | 6%-8% | 0%-1% |
| Ex 7010 | 8%-10% | 2%-4% | 2%-4% | 6%-8% | 1%-2% |
| Ex 7011 | 8%-10% | 2%-4% | 2%-4% | 6%-8% | 2%-4% |
| Ex 7012 | 8%-10% | 2%-4% | 2%-4% | 6%-8% | 4%-6% |
| Ex 7013 | 8%-10% | 2%-4% | 2%-4% | 6%-8% | 6%-8% |
| Ex 7014 | 8%-10% | 2%-4% | 2%-4% | 6%-8% | 8%-10% |
| Ex 7015 | 8%-10% | 2%-4% | 2%-4% | 8%-10% | 0%-1% |
| Ex 7016 | 8%-10% | 2%-4% | 2%-4% | 8%-10% | 1%-2% |
| Ex 7017 | 8%-10% | 2%-4% | 2%-4% | 8%-10% | 2%-4% |
| Ex 7018 | 8%-10% | 2%-4% | 2%-4% | 8%-10% | 4%-6% |
| Ex 7019 | 8%-10% | 2%-4% | 2%-4% | 8%-10% | 6%-8% |
| Ex 7020 | 8%-10% | 2%-4% | 2%-4% | 8%-10% | 8%-10% |
| Ex 7021 | 8%-10% | 2%-4% | 4%-6% | 0%-1% | 0%-1% |
| Ex 7022 | 8%-10% | 2%-4% | 4%-6% | 0%-1% | 1%-2% |
| Ex 7023 | 8%-10% | 2%-4% | 4%-6% | 0%-1% | 2%-4% |
| Ex 7024 | 8%-10% | 2%-4% | 4%-6% | 0%-1% | 4%-6% |
| Ex 7025 | 8%-10% | 2%-4% | 4%-6% | 0%-1% | 6%-8% |
| Ex 7026 | 8%-10% | 2%-4% | 4%-6% | 0%-1% | 8%-10% |
| Ex 7027 | 8%-10% | 2%-4% | 4%-6% | 1%-2% | 0%-1% |
| Ex 7028 | 8%-10% | 2%-4% | 4%-6% | 1%-2% | 1%-2% |
| Ex 7029 | 8%-10% | 2%-4% | 4%-6% | 1%-2% | 2%-4% |
| Ex 7030 | 8%-10% | 2%-4% | 4%-6% | 1%-2% | 4%-6% |
| Ex 7031 | 8%-10% | 2%-4% | 4%-6% | 1%-2% | 6%-8% |
| Ex 7032 | 8%-10% | 2%-4% | 4%-6% | 1%-2% | 8%-10% |
| Ex 7033 | 8%-10% | 2%-4% | 4%-6% | 2%-4% | 0%-1% |
| Ex 7034 | 8%-10% | 2%-4% | 4%-6% | 2%-4% | 1%-2% |
| Ex 7035 | 8%-10% | 2%-4% | 4%-6% | 2%-4% | 2%-4% |
| Ex 7036 | 8%-10% | 2%-4% | 4%-6% | 2%-4% | 4%-6% |
| Ex 7037 | 8%-10% | 2%-4% | 4%-6% | 2%-4% | 6%-8% |
| Ex 7038 | 8%-10% | 2%-4% | 4%-6% | 2%-4% | 8%-10% |
| Ex 7039 | 8%-10% | 2%-4% | 4%-6% | 4%-6% | 0%-1% |
| Ex 7040 | 8%-10% | 2%-4% | 4%-6% | 4%-6% | 1%-2% |
| Ex 7041 | 8%-10% | 2%-4% | 4%-6% | 4%-6% | 2%-4% |
| Ex 7042 | 8%-10% | 2%-4% | 4%-6% | 4%-6% | 4%-6% |
| Ex 7043 | 8%-10% | 2%-4% | 4%-6% | 4%-6% | 6%-8% |
| Ex 7044 | 8%-10% | 2%-4% | 4%-6% | 4%-6% | 8%-10% |
| Ex 7045 | 8%-10% | 2%-4% | 4%-6% | 6%-8% | 0%-1% |
| Ex 7046 | 8%-10% | 2%-4% | 4%-6% | 6%-8% | 1%-2% |
| Ex 7047 | 8%-10% | 2%-4% | 4%-6% | 6%-8% | 2%-4% |
| Ex 7048 | 8%-10% | 2%-4% | 4%-6% | 6%-8% | 4%-6% |
| Ex 7049 | 8%-10% | 2%-4% | 4%-6% | 6%-8% | 6%-8% |
| Ex 7050 | 8%-10% | 2%-4% | 4%-6% | 6%-8% | 8%-10% |
| Ex 7051 | 8%-10% | 2%-4% | 4%-6% | 8%-10% | 0%-1% |
| Ex 7052 | 8%-10% | 2%-4% | 4%-6% | 8%-10% | 1%-2% |
| Ex 7053 | 8%-10% | 2%-4% | 4%-6% | 8%-10% | 2%-4% |
| Ex 7054 | 8%-10% | 2%-4% | 4%-6% | 8%-10% | 4%-6% |
| Ex 7055 | 8%-10% | 2%-4% | 4%-6% | 8%-10% | 6%-8% |
| Ex 7056 | 8%-10% | 2%-4% | 4%-6% | 8%-10% | 8%-10% |
| Ex 7057 | 8%-10% | 2%-4% | 6%-8% | 0%-1% | 0%-1% |
| Ex 7058 | 8%-10% | 2%-4% | 6%-8% | 0%-1% | 1%-2% |
| Ex 7059 | 8%-10% | 2%-4% | 6%-8% | 0%-1% | 2%-4% |
| Ex 7060 | 8%-10% | 2%-4% | 6%-8% | 0%-1% | 4%-6% |
| Ex 7061 | 8%-10% | 2%-4% | 6%-8% | 0%-1% | 6%-8% |
| Ex 7062 | 8%-10% | 2%-4% | 6%-8% | 0%-1% | 8%-10% |
| Ex 7063 | 8%-10% | 2%-4% | 6%-8% | 1%-2% | 0%-1% |
| Ex 7064 | 8%-10% | 2%-4% | 6%-8% | 1%-2% | 1%-2% |
| Ex 7065 | 8%-10% | 2%-4% | 6%-8% | 1%-2% | 2%-4% |
| Ex 7066 | 8%-10% | 2%-4% | 6%-8% | 1%-2% | 4%-6% |
| Ex 7067 | 8%-10% | 2%-4% | 6%-8% | 1%-2% | 6%-8% |
| Ex 7068 | 8%-10% | 2%-4% | 6%-8% | 1%-2% | 8%-10% |
| Ex 7069 | 8%-10% | 2%-4% | 6%-8% | 2%-4% | 0%-1% |
| Ex 7070 | 8%-10% | 2%-4% | 6%-8% | 2%-4% | 1%-2% |
| Ex 7071 | 8%-10% | 2%-4% | 6%-8% | 2%-4% | 2%-4% |
| Ex 7072 | 8%-10% | 2%-4% | 6%-8% | 2%-4% | 4%-6% |
| Ex 7073 | 8%-10% | 2%-4% | 6%-8% | 2%-4% | 6%-8% |
| Ex 7074 | 8%-10% | 2%-4% | 6%-8% | 2%-4% | 8%-10% |
| Ex 7075 | 8%-10% | 2%-4% | 6%-8% | 4%-6% | 0%-1% |
| Ex 7076 | 8%-10% | 2%-4% | 6%-8% | 4%-6% | 1%-2% |
| Ex 7077 | 8%-10% | 2%-4% | 6%-8% | 4%-6% | 2%-4% |
| Ex 7078 | 8%-10% | 2%-4% | 6%-8% | 4%-6% | 4%-6% |
| Ex 7079 | 8%-10% | 2%-4% | 6%-8% | 4%-6% | 6%-8% |
| Ex 7080 | 8%-10% | 2%-4% | 6%-8% | 4%-6% | 8%-10% |
| Ex 7081 | 8%-10% | 2%-4% | 6%-8% | 6%-8% | 0%-1% |
| Ex 7082 | 8%-10% | 2%-4% | 6%-8% | 6%-8% | 1%-2% |
| Ex 7083 | 8%-10% | 2%-4% | 6%-8% | 6%-8% | 2%-4% |
| Ex 7084 | 8%-10% | 2%-4% | 6%-8% | 6%-8% | 4%-6% |
| Ex 7085 | 8%-10% | 2%-4% | 6%-8% | 6%-8% | 6%-8% |
| Ex 7086 | 8%-10% | 2%-4% | 6%-8% | 6%-8% | 8%-10% |
| Ex 7087 | 8%-10% | 2%-4% | 6%-8% | 8%-10% | 0%-1% |
| Ex 7088 | 8%-10% | 2%-4% | 6%-8% | 8%-10% | 1%-2% |
| Ex 7089 | 8%-10% | 2%-4% | 6%-8% | 8%-10% | 2%-4% |
| Ex 7090 | 8%-10% | 2%-4% | 6%-8% | 8%-10% | 4%-6% |
| Ex 7091 | 8%-10% | 2%-4% | 6%-8% | 8%-10% | 6%-8% |
| Ex 7092 | 8%-10% | 2%-4% | 6%-8% | 8%-10% | 8%-10% |
| Ex 7093 | 8%-10% | 2%-4% | 8%-10% | 0%-1% | 0%-1% |
| Ex 7094 | 8%-10% | 2%-4% | 8%-10% | 0%-1% | 1%-2% |
| Ex 7095 | 8%-10% | 2%-4% | 8%-10% | 0%-1% | 2%-4% |
| Ex 7096 | 8%-10% | 2%-4% | 8%-10% | 0%-1% | 4%-6% |
| Ex 7097 | 8%-10% | 2%-4% | 8%-10% | 0%-1% | 6%-8% |
| Ex 7098 | 8%-10% | 2%-4% | 8%-10% | 0%-1% | 8%-10% |
| Ex 7099 | 8%-10% | 2%-4% | 8%-10% | 1%-2% | 0%-1% |
| Ex 7100 | 8%-10% | 2%-4% | 8%-10% | 1%-2% | 1%-2% |
| Ex 7101 | 8%-10% | 2%-4% | 8%-10% | 1%-2% | 2%-4% |
| Ex 7102 | 8%-10% | 2%-4% | 8%-10% | 1%-2% | 4%-6% |
| Ex 7103 | 8%-10% | 2%-4% | 8%-10% | 1%-2% | 6%-8% |
| Ex 7104 | 8%-10% | 2%-4% | 8%-10% | 1%-2% | 8%-10% |
| Ex 7105 | 8%-10% | 2%-4% | 8%-10% | 2%-4% | 0%-1% |
| Ex 7106 | 8%-10% | 2%-4% | 8%-10% | 2%-4% | 1%-2% |
| Ex 7107 | 8%-10% | 2%-4% | 8%-10% | 2%-4% | 2%-4% |
| Ex 7108 | 8%-10% | 2%-4% | 8%-10% | 2%-4% | 4%-6% |
| Ex 7109 | 8%-10% | 2%-4% | 8%-10% | 2%-4% | 6%-8% |
| Ex 7110 | 8%-10% | 2%-4% | 8%-10% | 2%-4% | 8%-10% |
| Ex 7111 | 8%-10% | 2%-4% | 8%-10% | 4%-6% | 0%-1% |
| Ex 7112 | 8%-10% | 2%-4% | 8%-10% | 4%-6% | 1%-2% |
| Ex 7113 | 8%-10% | 2%-4% | 8%-10% | 4%-6% | 2%-4% |
| Ex 7114 | 8%-10% | 2%-4% | 8%-10% | 4%-6% | 4%-6% |
| Ex 7115 | 8%-10% | 2%-4% | 8%-10% | 4%-6% | 6%-8% |
| Ex 7116 | 8%-10% | 2%-4% | 8%-10% | 4%-6% | 8%-10% |
| Ex 7117 | 8%-10% | 2%-4% | 8%-10% | 6%-8% | 0%-1% |
| Ex 7118 | 8%-10% | 2%-4% | 8%-10% | 6%-8% | 1%-2% |
| Ex 7119 | 8%-10% | 2%-4% | 8%-10% | 6%-8% | 2%-4% |
| Ex 7120 | 8%-10% | 2%-4% | 8%-10% | 6%-8% | 4%-6% |
| Ex 7121 | 8%-10% | 2%-4% | 8%-10% | 6%-8% | 6%-8% |
| Ex 7122 | 8%-10% | 2%-4% | 8%-10% | 6%-8% | 8%-10% |
| Ex 7123 | 8%-10% | 2%-4% | 8%-10% | 8%-10% | 0%-1% |
| Ex 7124 | 8%-10% | 2%-4% | 8%-10% | 8%-10% | 1%-2% |
| Ex 7125 | 8%-10% | 2%-4% | 8%-10% | 8%-10% | 2%-4% |
| Ex 7126 | 8%-10% | 2%-4% | 8%-10% | 8%-10% | 4%-6% |
| Ex 7127 | 8%-10% | 2%-4% | 8%-10% | 8%-10% | 6%-8% |
| Ex 7128 | 8%-10% | 2%-4% | 8%-10% | 8%-10% | 8%-10% |
| Ex 7129 | 8%-10% | 4%-6% | 0%-1% | 0%-1% | 0%-1% |
| Ex 7130 | 8%-10% | 4%-6% | 0%-1% | 0%-1% | 1%-2% |
| Ex 7131 | 8%-10% | 4%-6% | 0%-1% | 0%-1% | 2%-4% |
| Ex 7132 | 8%-10% | 4%-6% | 0%-1% | 0%-1% | 4%-6% |
| Ex 7133 | 8%-10% | 4%-6% | 0%-1% | 0%-1% | 6%-8% |
| Ex 7134 | 8%-10% | 4%-6% | 0%-1% | 0%-1% | 8%-10% |
| Ex 7135 | 8%-10% | 4%-6% | 0%-1% | 1%-2% | 0%-1% |
| Ex 7136 | 8%-10% | 4%-6% | 0%-1% | 1%-2% | 1%-2% |
| Ex 7137 | 8%-10% | 4%-6% | 0%-1% | 1%-2% | 2%-4% |
| Ex 7138 | 8%-10% | 4%-6% | 0%-1% | 1%-2% | 4%-6% |
| Ex 7139 | 8%-10% | 4%-6% | 0%-1% | 1%-2% | 6%-8% |
| Ex 7140 | 8%-10% | 4%-6% | 0%-1% | 1%-2% | 8%-10% |
| Ex 7141 | 8%-10% | 4%-6% | 0%-1% | 2%-4% | 0%-1% |
| Ex 7142 | 8%-10% | 4%-6% | 0%-1% | 2%-4% | 1%-2% |
| Ex 7143 | 8%-10% | 4%-6% | 0%-1% | 2%-4% | 2%-4% |
| Ex 7144 | 8%-10% | 4%-6% | 0%-1% | 2%-4% | 4%-6% |
| Ex 7145 | 8%-10% | 4%-6% | 0%-1% | 2%-4% | 6%-8% |
| Ex 7146 | 8%-10% | 4%-6% | 0%-1% | 2%-4% | 8%-10% |
| Ex 7147 | 8%-10% | 4%-6% | 0%-1% | 4%-6% | 0%-1% |
| Ex 7148 | 8%-10% | 4%-6% | 0%-1% | 4%-6% | 1%-2% |
| Ex 7149 | 8%-10% | 4%-6% | 0%-1% | 4%-6% | 2%-4% |
| Ex 7150 | 8%-10% | 4%-6% | 0%-1% | 4%-6% | 4%-6% |
| Ex 7151 | 8%-10% | 4%-6% | 0%-1% | 4%-6% | 6%-8% |
| Ex 7152 | 8%-10% | 4%-6% | 0%-1% | 4%-6% | 8%-10% |
| Ex 7153 | 8%-10% | 4%-6% | 0%-1% | 6%-8% | 0%-1% |
| Ex 7154 | 8%-10% | 4%-6% | 0%-1% | 6%-8% | 1%-2% |
| Ex 7155 | 8%-10% | 4%-6% | 0%-1% | 6%-8% | 2%-4% |
| Ex 7156 | 8%-10% | 4%-6% | 0%-1% | 6%-8% | 4%-6% |
| Ex 7157 | 8%-10% | 4%-6% | 0%-1% | 6%-8% | 6%-8% |
| Ex 7158 | 8%-10% | 4%-6% | 0%-1% | 6%-8% | 8%-10% |
| Ex 7159 | 8%-10% | 4%-6% | 0%-1% | 8%-10% | 0%-1% |
| Ex 7160 | 8%-10% | 4%-6% | 0%-1% | 8%-10% | 1%-2% |
| Ex 7161 | 8%-10% | 4%-6% | 0%-1% | 8%-10% | 2%-4% |
| Ex 7162 | 8%-10% | 4%-6% | 0%-1% | 8%-10% | 4%-6% |
| Ex 7163 | 8%-10% | 4%-6% | 0%-1% | 8%-10% | 6%-8% |
| Ex 7164 | 8%-10% | 4%-6% | 0%-1% | 8%-10% | 8%-10% |
| Ex 7165 | 8%-10% | 4%-6% | 1%-2% | 0%-1% | 0%-1% |
| Ex 7166 | 8%-10% | 4%-6% | 1%-2% | 0%-1% | 1%-2% |
| Ex 7167 | 8%-10% | 4%-6% | 1%-2% | 0%-1% | 2%-4% |
| Ex 7168 | 8%-10% | 4%-6% | 1%-2% | 0%-1% | 4%-6% |
| Ex 7169 | 8%-10% | 4%-6% | 1%-2% | 0%-1% | 6%-8% |
| Ex 7170 | 8%-10% | 4%-6% | 1%-2% | 0%-1% | 8%-10% |
| Ex 7171 | 8%-10% | 4%-6% | 1%-2% | 1%-2% | 0%-1% |
| Ex 7172 | 8%-10% | 4%-6% | 1%-2% | 1%-2% | 1%-2% |
| Ex 7173 | 8%-10% | 4%-6% | 1%-2% | 1%-2% | 2%-4% |
| Ex 7174 | 8%-10% | 4%-6% | 1%-2% | 1%-2% | 4%-6% |
| Ex 7175 | 8%-10% | 4%-6% | 1%-2% | 1%-2% | 6%-8% |
| Ex 7176 | 8%-10% | 4%-6% | 1%-2% | 1%-2% | 8%-10% |
| Ex 7177 | 8%-10% | 4%-6% | 1%-2% | 2%-4% | 0%-1% |
| Ex 7178 | 8%-10% | 4%-6% | 1%-2% | 2%-4% | 1%-2% |
| Ex 7179 | 8%-10% | 4%-6% | 1%-2% | 2%-4% | 2%-4% |
| Ex 7180 | 8%-10% | 4%-6% | 1%-2% | 2%-4% | 4%-6% |
| Ex 7181 | 8%-10% | 4%-6% | 1%-2% | 2%-4% | 6%-8% |
| Ex 7182 | 8%-10% | 4%-6% | 1%-2% | 2%-4% | 8%-10% |
| Ex 7183 | 8%-10% | 4%-6% | 1%-2% | 4%-6% | 0%-1% |
| Ex 7184 | 8%-10% | 4%-6% | 1%-2% | 4%-6% | 1%-2% |
| Ex 7185 | 8%-10% | 4%-6% | 1%-2% | 4%-6% | 2%-4% |
| Ex 7186 | 8%-10% | 4%-6% | 1%-2% | 4%-6% | 4%-6% |
| Ex 7187 | 8%-10% | 4%-6% | 1%-2% | 4%-6% | 6%-8% |
| Ex 7188 | 8%-10% | 4%-6% | 1%-2% | 4%-6% | 8%-10% |
| Ex 7189 | 8%-10% | 4%-6% | 1%-2% | 6%-8% | 0%-1% |
| Ex 7190 | 8%-10% | 4%-6% | 1%-2% | 6%-8% | 1%-2% |
| Ex 7191 | 8%-10% | 4%-6% | 1%-2% | 6%-8% | 2%-4% |
| Ex 7192 | 8%-10% | 4%-6% | 1%-2% | 6%-8% | 4%-6% |
| Ex 7193 | 8%-10% | 4%-6% | 1%-2% | 6%-8% | 6%-8% |
| Ex 7194 | 8%-10% | 4%-6% | 1%-2% | 6%-8% | 8%-10% |
| Ex 7195 | 8%-10% | 4%-6% | 1%-2% | 8%-10% | 0%-1% |
| Ex 7196 | 8%-10% | 4%-6% | 1%-2% | 8%-10% | 1%-2% |
| Ex 7197 | 8%-10% | 4%-6% | 1%-2% | 8%-10% | 2%-4% |
| Ex 7198 | 8%-10% | 4%-6% | 1%-2% | 8%-10% | 4%-6% |
| Ex 7199 | 8%-10% | 4%-6% | 1%-2% | 8%-10% | 6%-8% |
| Ex 7200 | 8%-10% | 4%-6% | 1%-2% | 8%-10% | 8%-10% |
| Ex 7201 | 8%-10% | 4%-6% | 2%-4% | 0%-1% | 0%-1% |
| Ex 7202 | 8%-10% | 4%-6% | 2%-4% | 0%-1% | 1%-2% |
| Ex 7203 | 8%-10% | 4%-6% | 2%-4% | 0%-1% | 2%-4% |
| Ex 7204 | 8%-10% | 4%-6% | 2%-4% | 0%-1% | 4%-6% |
| Ex 7205 | 8%-10% | 4%-6% | 2%-4% | 0%-1% | 6%-8% |
| Ex 7206 | 8%-10% | 4%-6% | 2%-4% | 0%-1% | 8%-10% |
| Ex 7207 | 8%-10% | 4%-6% | 2%-4% | 1%-2% | 0%-1% |
| Ex 7208 | 8%-10% | 4%-6% | 2%-4% | 1%-2% | 1%-2% |
| Ex 7209 | 8%-10% | 4%-6% | 2%-4% | 1%-2% | 2%-4% |
| Ex 7210 | 8%-10% | 4%-6% | 2%-4% | 1%-2% | 4%-6% |
| Ex 7211 | 8%-10% | 4%-6% | 2%-4% | 1%-2% | 6%-8% |
| Ex 7212 | 8%-10% | 4%-6% | 2%-4% | 1%-2% | 8%-10% |
| Ex 7213 | 8%-10% | 4%-6% | 2%-4% | 2%-4% | 0%-1% |
| Ex 7214 | 8%-10% | 4%-6% | 2%-4% | 2%-4% | 1%-2% |
| Ex 7215 | 8%-10% | 4%-6% | 2%-4% | 2%-4% | 2%-4% |
| Ex 7216 | 8%-10% | 4%-6% | 2%-4% | 2%-4% | 4%-6% |
| Ex 7217 | 8%-10% | 4%-6% | 2%-4% | 2%-4% | 6%-8% |
| Ex 7218 | 8%-10% | 4%-6% | 2%-4% | 2%-4% | 8%-10% |
| Ex 7219 | 8%-10% | 4%-6% | 2%-4% | 4%-6% | 0%-1% |
| Ex 7220 | 8%-10% | 4%-6% | 2%-4% | 4%-6% | 1%-2% |
| Ex 7221 | 8%-10% | 4%-6% | 2%-4% | 4%-6% | 2%-4% |
| Ex 7222 | 8%-10% | 4%-6% | 2%-4% | 4%-6% | 4%-6% |
| Ex 7223 | 8%-10% | 4%-6% | 2%-4% | 4%-6% | 6%-8% |
| Ex 7224 | 8%-10% | 4%-6% | 2%-4% | 4%-6% | 8%-10% |
| Ex 7225 | 8%-10% | 4%-6% | 2%-4% | 6%-8% | 0%-1% |
| Ex 7226 | 8%-10% | 4%-6% | 2%-4% | 6%-8% | 1%-2% |
| Ex 7227 | 8%-10% | 4%-6% | 2%-4% | 6%-8% | 2%-4% |
| Ex 7228 | 8%-10% | 4%-6% | 2%-4% | 6%-8% | 4%-6% |
| Ex 7229 | 8%-10% | 4%-6% | 2%-4% | 6%-8% | 6%-8% |
| Ex 7230 | 8%-10% | 4%-6% | 2%-4% | 6%-8% | 8%-10% |
| Ex 7231 | 8%-10% | 4%-6% | 2%-4% | 8%-10% | 0%-1% |
| Ex 7232 | 8%-10% | 4%-6% | 2%-4% | 8%-10% | 1%-2% |
| Ex 7233 | 8%-10% | 4%-6% | 2%-4% | 8%-10% | 2%-4% |
| Ex 7234 | 8%-10% | 4%-6% | 2%-4% | 8%-10% | 4%-6% |
| Ex 7235 | 8%-10% | 4%-6% | 2%-4% | 8%-10% | 6%-8% |
| Ex 7236 | 8%-10% | 4%-6% | 2%-4% | 8%-10% | 8%-10% |
| Ex 7237 | 8%-10% | 4%-6% | 4%-6% | 0%-1% | 0%-1% |
| Ex 7238 | 8%-10% | 4%-6% | 4%-6% | 0%-1% | 1%-2% |
| Ex 7239 | 8%-10% | 4%-6% | 4%-6% | 0%-1% | 2%-4% |
| Ex 7240 | 8%-10% | 4%-6% | 4%-6% | 0%-1% | 4%-6% |
| Ex 7241 | 8%-10% | 4%-6% | 4%-6% | 0%-1% | 6%-8% |
| Ex 7242 | 8%-10% | 4%-6% | 4%-6% | 0%-1% | 8%-10% |
| Ex 7243 | 8%-10% | 4%-6% | 4%-6% | 1%-2% | 0%-1% |
| Ex 7244 | 8%-10% | 4%-6% | 4%-6% | 1%-2% | 1%-2% |
| Ex 7245 | 8%-10% | 4%-6% | 4%-6% | 1%-2% | 2%-4% |
| Ex 7246 | 8%-10% | 4%-6% | 4%-6% | 1%-2% | 4%-6% |
| Ex 7247 | 8%-10% | 4%-6% | 4%-6% | 1%-2% | 6%-8% |
| Ex 7248 | 8%-10% | 4%-6% | 4%-6% | 1%-2% | 8%-10% |
| Ex 7249 | 8%-10% | 4%-6% | 4%-6% | 2%-4% | 0%-1% |
| Ex 7250 | 8%-10% | 4%-6% | 4%-6% | 2%-4% | 1%-2% |
| Ex 7251 | 8%-10% | 4%-6% | 4%-6% | 2%-4% | 2%-4% |
| Ex 7252 | 8%-10% | 4%-6% | 4%-6% | 2%-4% | 4%-6% |
| Ex 7253 | 8%-10% | 4%-6% | 4%-6% | 2%-4% | 6%-8% |
| Ex 7254 | 8%-10% | 4%-6% | 4%-6% | 2%-4% | 8%-10% |
| Ex 7255 | 8%-10% | 4%-6% | 4%-6% | 4%-6% | 0%-1% |
| Ex 7256 | 8%-10% | 4%-6% | 4%-6% | 4%-6% | 1%-2% |
| Ex 7257 | 8%-10% | 4%-6% | 4%-6% | 4%-6% | 2%-4% |
| Ex 7258 | 8%-10% | 4%-6% | 4%-6% | 4%-6% | 4%-6% |
| Ex 7259 | 8%-10% | 4%-6% | 4%-6% | 4%-6% | 6%-8% |
| Ex 7260 | 8%-10% | 4%-6% | 4%-6% | 4%-6% | 8%-10% |
| Ex 7261 | 8%-10% | 4%-6% | 4%-6% | 6%-8% | 0%-1% |
| Ex 7262 | 8%-10% | 4%-6% | 4%-6% | 6%-8% | 1%-2% |
| Ex 7263 | 8%-10% | 4%-6% | 4%-6% | 6%-8% | 2%-4% |
| Ex 7264 | 8%-10% | 4%-6% | 4%-6% | 6%-8% | 4%-6% |
| Ex 7265 | 8%-10% | 4%-6% | 4%-6% | 6%-8% | 6%-8% |
| Ex 7266 | 8%-10% | 4%-6% | 4%-6% | 6%-8% | 8%-10% |
| Ex 7267 | 8%-10% | 4%-6% | 4%-6% | 8%-10% | 0%-1% |
| Ex 7268 | 8%-10% | 4%-6% | 4%-6% | 8%-10% | 1%-2% |
| Ex 7269 | 8%-10% | 4%-6% | 4%-6% | 8%-10% | 2%-4% |
| Ex 7270 | 8%-10% | 4%-6% | 4%-6% | 8%-10% | 4%-6% |
| Ex 7271 | 8%-10% | 4%-6% | 4%-6% | 8%-10% | 6%-8% |
| Ex 7272 | 8%-10% | 4%-6% | 4%-6% | 8%-10% | 8%-10% |
| Ex 7273 | 8%-10% | 4%-6% | 6%-8% | 0%-1% | 0%-1% |
| Ex 7274 | 8%-10% | 4%-6% | 6%-8% | 0%-1% | 1%-2% |
| Ex 7275 | 8%-10% | 4%-6% | 6%-8% | 0%-1% | 2%-4% |
| Ex 7276 | 8%-10% | 4%-6% | 6%-8% | 0%-1% | 4%-6% |
| Ex 7277 | 8%-10% | 4%-6% | 6%-8% | 0%-1% | 6%-8% |
| Ex 7278 | 8%-10% | 4%-6% | 6%-8% | 0%-1% | 8%-10% |
| Ex 7279 | 8%-10% | 4%-6% | 6%-8% | 1%-2% | 0%-1% |
| Ex 7280 | 8%-10% | 4%-6% | 6%-8% | 1%-2% | 1%-2% |
| Ex 7281 | 8%-10% | 4%-6% | 6%-8% | 1%-2% | 2%-4% |
| Ex 7282 | 8%-10% | 4%-6% | 6%-8% | 1%-2% | 4%-6% |
| Ex 7283 | 8%-10% | 4%-6% | 6%-8% | 1%-2% | 6%-8% |
| Ex 7284 | 8%-10% | 4%-6% | 6%-8% | 1%-2% | 8%-10% |
| Ex 7285 | 8%-10% | 4%-6% | 6%-8% | 2%-4% | 0%-1% |
| Ex 7286 | 8%-10% | 4%-6% | 6%-8% | 2%-4% | 1%-2% |
| Ex 7287 | 8%-10% | 4%-6% | 6%-8% | 2%-4% | 2%-4% |
| Ex 7288 | 8%-10% | 4%-6% | 6%-8% | 2%-4% | 4%-6% |
| Ex 7289 | 8%-10% | 4%-6% | 6%-8% | 2%-4% | 6%-8% |
| Ex 7290 | 8%-10% | 4%-6% | 6%-8% | 2%-4% | 8%-10% |
| Ex 7291 | 8%-10% | 4%-6% | 6%-8% | 4%-6% | 0%-1% |
| Ex 7292 | 8%-10% | 4%-6% | 6%-8% | 4%-6% | 1%-2% |
| Ex 7293 | 8%-10% | 4%-6% | 6%-8% | 4%-6% | 2%-4% |
| Ex 7294 | 8%-10% | 4%-6% | 6%-8% | 4%-6% | 4%-6% |
| Ex 7295 | 8%-10% | 4%-6% | 6%-8% | 4%-6% | 6%-8% |
| Ex 7296 | 8%-10% | 4%-6% | 6%-8% | 4%-6% | 8%-10% |
| Ex 7297 | 8%-10% | 4%-6% | 6%-8% | 6%-8% | 0%-1% |
| Ex 7298 | 8%-10% | 4%-6% | 6%-8% | 6%-8% | 1%-2% |
| Ex 7299 | 8%-10% | 4%-6% | 6%-8% | 6%-8% | 2%-4% |
| Ex 7300 | 8%-10% | 4%-6% | 6%-8% | 6%-8% | 4%-6% |
| Ex 7301 | 8%-10% | 4%-6% | 6%-8% | 6%-8% | 6%-8% |
| Ex 7302 | 8%-10% | 4%-6% | 6%-8% | 6%-8% | 8%-10% |
| Ex 7303 | 8%-10% | 4%-6% | 6%-8% | 8%-10% | 0%-1% |
| Ex 7304 | 8%-10% | 4%-6% | 6%-8% | 8%-10% | 1%-2% |
| Ex 7305 | 8%-10% | 4%-6% | 6%-8% | 8%-10% | 2%-4% |
| Ex 7306 | 8%-10% | 4%-6% | 6%-8% | 8%-10% | 4%-6% |
| Ex 7307 | 8%-10% | 4%-6% | 6%-8% | 8%-10% | 6%-8% |
| Ex 7308 | 8%-10% | 4%-6% | 6%-8% | 8%-10% | 8%-10% |
| Ex 7309 | 8%-10% | 4%-6% | 8%-10% | 0%-1% | 0%-1% |
| Ex 7310 | 8%-10% | 4%-6% | 8%-10% | 0%-1% | 1%-2% |
| Ex 7311 | 8%-10% | 4%-6% | 8%-10% | 0%-1% | 2%-4% |
| Ex 7312 | 8%-10% | 4%-6% | 8%-10% | 0%-1% | 4%-6% |
| Ex 7313 | 8%-10% | 4%-6% | 8%-10% | 0%-1% | 6%-8% |
| Ex 7314 | 8%-10% | 4%-6% | 8%-10% | 0%-1% | 8%-10% |
| Ex 7315 | 8%-10% | 4%-6% | 8%-10% | 1%-2% | 0%-1% |
| Ex 7316 | 8%-10% | 4%-6% | 8%-10% | 1%-2% | 1%-2% |
| Ex 7317 | 8%-10% | 4%-6% | 8%-10% | 1%-2% | 2%-4% |
| Ex 7318 | 8%-10% | 4%-6% | 8%-10% | 1%-2% | 4%-6% |
| Ex 7319 | 8%-10% | 4%-6% | 8%-10% | 1%-2% | 6%-8% |
| Ex 7320 | 8%-10% | 4%-6% | 8%-10% | 1%-2% | 8%-10% |
| Ex 7321 | 8%-10% | 4%-6% | 8%-10% | 2%-4% | 0%-1% |
| Ex 7322 | 8%-10% | 4%-6% | 8%-10% | 2%-4% | 1%-2% |
| Ex 7323 | 8%-10% | 4%-6% | 8%-10% | 2%-4% | 2%-4% |
| Ex 7324 | 8%-10% | 4%-6% | 8%-10% | 2%-4% | 4%-6% |
| Ex 7325 | 8%-10% | 4%-6% | 8%-10% | 2%-4% | 6%-8% |
| Ex 7326 | 8%-10% | 4%-6% | 8%-10% | 2%-4% | 8%-10% |
| Ex 7327 | 8%-10% | 4%-6% | 8%-10% | 4%-6% | 0%-1% |
| Ex 7328 | 8%-10% | 4%-6% | 8%-10% | 4%-6% | 1%-2% |
| Ex 7329 | 8%-10% | 4%-6% | 8%-10% | 4%-6% | 2%-4% |
| Ex 7330 | 8%-10% | 4%-6% | 8%-10% | 4%-6% | 4%-6% |
| Ex 7331 | 8%-10% | 4%-6% | 8%-10% | 4%-6% | 6%-8% |
| Ex 7332 | 8%-10% | 4%-6% | 8%-10% | 4%-6% | 8%-10% |
| Ex 7333 | 8%-10% | 4%-6% | 8%-10% | 6%-8% | 0%-1% |
| Ex 7334 | 8%-10% | 4%-6% | 8%-10% | 6%-8% | 1%-2% |
| Ex 7335 | 8%-10% | 4%-6% | 8%-10% | 6%-8% | 2%-4% |
| Ex 7336 | 8%-10% | 4%-6% | 8%-10% | 6%-8% | 4%-6% |
| Ex 7337 | 8%-10% | 4%-6% | 8%-10% | 6%-8% | 6%-8% |
| Ex 7338 | 8%-10% | 4%-6% | 8%-10% | 6%-8% | 8%-10% |
| Ex 7339 | 8%-10% | 4%-6% | 8%-10% | 8%-10% | 0%-1% |
| Ex 7340 | 8%-10% | 4%-6% | 8%-10% | 8%-10% | 1%-2% |
| Ex 7341 | 8%-10% | 4%-6% | 8%-10% | 8%-10% | 2%-4% |
| Ex 7342 | 8%-10% | 4%-6% | 8%-10% | 8%-10% | 4%-6% |
| Ex 7343 | 8%-10% | 4%-6% | 8%-10% | 8%-10% | 6%-8% |
| Ex 7344 | 8%-10% | 4%-6% | 8%-10% | 8%-10% | 8%-10% |
| Ex 7345 | 8%-10% | 6%-8% | 0%-1% | 0%-1% | 0%-1% |
| Ex 7346 | 8%-10% | 6%-8% | 0%-1% | 0%-1% | 1%-2% |
| Ex 7347 | 8%-10% | 6%-8% | 0%-1% | 0%-1% | 2%-4% |
| Ex 7348 | 8%-10% | 6%-8% | 0%-1% | 0%-1% | 4%-6% |
| Ex 7349 | 8%-10% | 6%-8% | 0%-1% | 0%-1% | 6%-8% |
| Ex 7350 | 8%-10% | 6%-8% | 0%-1% | 0%-1% | 8%-10% |
| Ex 7351 | 8%-10% | 6%-8% | 0%-1% | 1%-2% | 0%-1% |
| Ex 7352 | 8%-10% | 6%-8% | 0%-1% | 1%-2% | 1%-2% |
| Ex 7353 | 8%-10% | 6%-8% | 0%-1% | 1%-2% | 2%-4% |
| Ex 7354 | 8%-10% | 6%-8% | 0%-1% | 1%-2% | 4%-6% |
| Ex 7355 | 8%-10% | 6%-8% | 0%-1% | 1%-2% | 6%-8% |
| Ex 7356 | 8%-10% | 6%-8% | 0%-1% | 1%-2% | 8%-10% |
| Ex 7357 | 8%-10% | 6%-8% | 0%-1% | 2%-4% | 0%-1% |
| Ex 7358 | 8%-10% | 6%-8% | 0%-1% | 2%-4% | 1%-2% |
| Ex 7359 | 8%-10% | 6%-8% | 0%-1% | 2%-4% | 2%-4% |
| Ex 7360 | 8%-10% | 6%-8% | 0%-1% | 2%-4% | 4%-6% |
| Ex 7361 | 8%-10% | 6%-8% | 0%-1% | 2%-4% | 6%-8% |
| Ex 7362 | 8%-10% | 6%-8% | 0%-1% | 2%-4% | 8%-10% |
| Ex 7363 | 8%-10% | 6%-8% | 0%-1% | 4%-6% | 0%-1% |
| Ex 7364 | 8%-10% | 6%-8% | 0%-1% | 4%-6% | 1%-2% |
| Ex 7365 | 8%-10% | 6%-8% | 0%-1% | 4%-6% | 2%-4% |
| Ex 7366 | 8%-10% | 6%-8% | 0%-1% | 4%-6% | 4%-6% |
| Ex 7367 | 8%-10% | 6%-8% | 0%-1% | 4%-6% | 6%-8% |
| Ex 7368 | 8%-10% | 6%-8% | 0%-1% | 4%-6% | 8%-10% |
| Ex 7369 | 8%-10% | 6%-8% | 0%-1% | 6%-8% | 0%-1% |
| Ex 7370 | 8%-10% | 6%-8% | 0%-1% | 6%-8% | 1%-2% |
| Ex 7371 | 8%-10% | 6%-8% | 0%-1% | 6%-8% | 2%-4% |
| Ex 7372 | 8%-10% | 6%-8% | 0%-1% | 6%-8% | 4%-6% |
| Ex 7373 | 8%-10% | 6%-8% | 0%-1% | 6%-8% | 6%-8% |
| Ex 7374 | 8%-10% | 6%-8% | 0%-1% | 6%-8% | 8%-10% |
| Ex 7375 | 8%-10% | 6%-8% | 0%-1% | 8%-10% | 0%-1% |
| Ex 7376 | 8%-10% | 6%-8% | 0%-1% | 8%-10% | 1%-2% |
| Ex 7377 | 8%-10% | 6%-8% | 0%-1% | 8%-10% | 2%-4% |
| Ex 7378 | 8%-10% | 6%-8% | 0%-1% | 8%-10% | 4%-6% |
| Ex 7379 | 8%-10% | 6%-8% | 0%-1% | 8%-10% | 6%-8% |
| Ex 7380 | 8%-10% | 6%-8% | 0%-1% | 8%-10% | 8%-10% |
| Ex 7381 | 8%-10% | 6%-8% | 1%-2% | 0%-1% | 0%-1% |
| Ex 7382 | 8%-10% | 6%-8% | 1%-2% | 0%-1% | 1%-2% |
| Ex 7383 | 8%-10% | 6%-8% | 1%-2% | 0%-1% | 2%-4% |
| Ex 7384 | 8%-10% | 6%-8% | 1%-2% | 0%-1% | 4%-6% |
| Ex 7385 | 8%-10% | 6%-8% | 1%-2% | 0%-1% | 6%-8% |
| Ex 7386 | 8%-10% | 6%-8% | 1%-2% | 0%-1% | 8%-10% |
| Ex 7387 | 8%-10% | 6%-8% | 1%-2% | 1%-2% | 0%-1% |
| Ex 7388 | 8%-10% | 6%-8% | 1%-2% | 1%-2% | 1%-2% |
| Ex 7389 | 8%-10% | 6%-8% | 1%-2% | 1%-2% | 2%-4% |
| Ex 7390 | 8%-10% | 6%-8% | 1%-2% | 1%-2% | 4%-6% |
| Ex 7391 | 8%-10% | 6%-8% | 1%-2% | 1%-2% | 6%-8% |
| Ex 7392 | 8%-10% | 6%-8% | 1%-2% | 1%-2% | 8%-10% |
| Ex 7393 | 8%-10% | 6%-8% | 1%-2% | 2%-4% | 0%-1% |
| Ex 7394 | 8%-10% | 6%-8% | 1%-2% | 2%-4% | 1%-2% |
| Ex 7395 | 8%-10% | 6%-8% | 1%-2% | 2%-4% | 2%-4% |
| Ex 7396 | 8%-10% | 6%-8% | 1%-2% | 2%-4% | 4%-6% |
| Ex 7397 | 8%-10% | 6%-8% | 1%-2% | 2%-4% | 6%-8% |
| Ex 7398 | 8%-10% | 6%-8% | 1%-2% | 2%-4% | 8%-10% |
| Ex 7399 | 8%-10% | 6%-8% | 1%-2% | 4%-6% | 0%-1% |
| Ex 7400 | 8%-10% | 6%-8% | 1%-2% | 4%-6% | 1%-2% |
| Ex 7401 | 8%-10% | 6%-8% | 1%-2% | 4%-6% | 2%-4% |
| Ex 7402 | 8%-10% | 6%-8% | 1%-2% | 4%-6% | 4%-6% |
| Ex 7403 | 8%-10% | 6%-8% | 1%-2% | 4%-6% | 6%-8% |
| Ex 7404 | 8%-10% | 6%-8% | 1%-2% | 4%-6% | 8%-10% |
| Ex 7405 | 8%-10% | 6%-8% | 1%-2% | 6%-8% | 0%-1% |
| Ex 7406 | 8%-10% | 6%-8% | 1%-2% | 6%-8% | 1%-2% |
| Ex 7407 | 8%-10% | 6%-8% | 1%-2% | 6%-8% | 2%-4% |
| Ex 7408 | 8%-10% | 6%-8% | 1%-2% | 6%-8% | 4%-6% |
| Ex 7409 | 8%-10% | 6%-8% | 1%-2% | 6%-8% | 6%-8% |
| Ex 7410 | 8%-10% | 6%-8% | 1%-2% | 6%-8% | 8%-10% |
| Ex 7411 | 8%-10% | 6%-8% | 1%-2% | 8%-10% | 0%-1% |
| Ex 7412 | 8%-10% | 6%-8% | 1%-2% | 8%-10% | 1%-2% |
| Ex 7413 | 8%-10% | 6%-8% | 1%-2% | 8%-10% | 2%-4% |
| Ex 7414 | 8%-10% | 6%-8% | 1%-2% | 8%-10% | 4%-6% |
| Ex 7415 | 8%-10% | 6%-8% | 1%-2% | 8%-10% | 6%-8% |
| Ex 7416 | 8%-10% | 6%-8% | 1%-2% | 8%-10% | 8%-10% |
| Ex 7417 | 8%-10% | 6%-8% | 2%-4% | 0%-1% | 0%-1% |
| Ex 7418 | 8%-10% | 6%-8% | 2%-4% | 0%-1% | 1%-2% |
| Ex 7419 | 8%-10% | 6%-8% | 2%-4% | 0%-1% | 2%-4% |
| Ex 7420 | 8%-10% | 6%-8% | 2%-4% | 0%-1% | 4%-6% |
| Ex 7421 | 8%-10% | 6%-8% | 2%-4% | 0%-1% | 6%-8% |
| Ex 7422 | 8%-10% | 6%-8% | 2%-4% | 0%-1% | 8%-10% |
| Ex 7423 | 8%-10% | 6%-8% | 2%-4% | 1%-2% | 0%-1% |
| Ex 7424 | 8%-10% | 6%-8% | 2%-4% | 1%-2% | 1%-2% |
| Ex 7425 | 8%-10% | 6%-8% | 2%-4% | 1%-2% | 2%-4% |
| Ex 7426 | 8%-10% | 6%-8% | 2%-4% | 1%-2% | 4%-6% |
| Ex 7427 | 8%-10% | 6%-8% | 2%-4% | 1%-2% | 6%-8% |
| Ex 7428 | 8%-10% | 6%-8% | 2%-4% | 1%-2% | 8%-10% |
| Ex 7429 | 8%-10% | 6%-8% | 2%-4% | 2%-4% | 0%-1% |
| Ex 7430 | 8%-10% | 6%-8% | 2%-4% | 2%-4% | 1%-2% |
| Ex 7431 | 8%-10% | 6%-8% | 2%-4% | 2%-4% | 2%-4% |
| Ex 7432 | 8%-10% | 6%-8% | 2%-4% | 2%-4% | 4%-6% |
| Ex 7433 | 8%-10% | 6%-8% | 2%-4% | 2%-4% | 6%-8% |
| Ex 7434 | 8%-10% | 6%-8% | 2%-4% | 2%-4% | 8%-10% |
| Ex 7435 | 8%-10% | 6%-8% | 2%-4% | 4%-6% | 0%-1% |
| Ex 7436 | 8%-10% | 6%-8% | 2%-4% | 4%-6% | 1%-2% |
| Ex 7437 | 8%-10% | 6%-8% | 2%-4% | 4%-6% | 2%-4% |
| Ex 7438 | 8%-10% | 6%-8% | 2%-4% | 4%-6% | 4%-6% |
| Ex 7439 | 8%-10% | 6%-8% | 2%-4% | 4%-6% | 6%-8% |
| Ex 7440 | 8%-10% | 6%-8% | 2%-4% | 4%-6% | 8%-10% |
| Ex 7441 | 8%-10% | 6%-8% | 2%-4% | 6%-8% | 0%-1% |
| Ex 7442 | 8%-10% | 6%-8% | 2%-4% | 6%-8% | 1%-2% |
| Ex 7443 | 8%-10% | 6%-8% | 2%-4% | 6%-8% | 2%-4% |
| Ex 7444 | 8%-10% | 6%-8% | 2%-4% | 6%-8% | 4%-6% |
| Ex 7445 | 8%-10% | 6%-8% | 2%-4% | 6%-8% | 6%-8% |
| Ex 7446 | 8%-10% | 6%-8% | 2%-4% | 6%-8% | 8%-10% |
| Ex 7447 | 8%-10% | 6%-8% | 2%-4% | 8%-10% | 0%-1% |
| Ex 7448 | 8%-10% | 6%-8% | 2%-4% | 8%-10% | 1%-2% |
| Ex 7449 | 8%-10% | 6%-8% | 2%-4% | 8%-10% | 2%-4% |
| Ex 7450 | 8%-10% | 6%-8% | 2%-4% | 8%-10% | 4%-6% |
| Ex 7451 | 8%-10% | 6%-8% | 2%-4% | 8%-10% | 6%-8% |
| Ex 7452 | 8%-10% | 6%-8% | 2%-4% | 8%-10% | 8%-10% |
| Ex 7453 | 8%-10% | 6%-8% | 4%-6% | 0%-1% | 0%-1% |
| Ex 7454 | 8%-10% | 6%-8% | 4%-6% | 0%-1% | 1%-2% |
| Ex 7455 | 8%-10% | 6%-8% | 4%-6% | 0%-1% | 2%-4% |
| Ex 7456 | 8%-10% | 6%-8% | 4%-6% | 0%-1% | 4%-6% |
| Ex 7457 | 8%-10% | 6%-8% | 4%-6% | 0%-1% | 6%-8% |
| Ex 7458 | 8%-10% | 6%-8% | 4%-6% | 0%-1% | 8%-10% |
| Ex 7459 | 8%-10% | 6%-8% | 4%-6% | 1%-2% | 0%-1% |
| Ex 7460 | 8%-10% | 6%-8% | 4%-6% | 1%-2% | 1%-2% |
| Ex 7461 | 8%-10% | 6%-8% | 4%-6% | 1%-2% | 2%-4% |
| Ex 7462 | 8%-10% | 6%-8% | 4%-6% | 1%-2% | 4%-6% |
| Ex 7463 | 8%-10% | 6%-8% | 4%-6% | 1%-2% | 6%-8% |
| Ex 7464 | 8%-10% | 6%-8% | 4%-6% | 1%-2% | 8%-10% |
| Ex 7465 | 8%-10% | 6%-8% | 4%-6% | 2%-4% | 0%-1% |
| Ex 7466 | 8%-10% | 6%-8% | 4%-6% | 2%-4% | 1%-2% |
| Ex 7467 | 8%-10% | 6%-8% | 4%-6% | 2%-4% | 2%-4% |
| Ex 7468 | 8%-10% | 6%-8% | 4%-6% | 2%-4% | 4%-6% |
| Ex 7469 | 8%-10% | 6%-8% | 4%-6% | 2%-4% | 6%-8% |
| Ex 7470 | 8%-10% | 6%-8% | 4%-6% | 2%-4% | 8%-10% |
| Ex 7471 | 8%-10% | 6%-8% | 4%-6% | 4%-6% | 0%-1% |
| Ex 7472 | 8%-10% | 6%-8% | 4%-6% | 4%-6% | 1%-2% |
| Ex 7473 | 8%-10% | 6%-8% | 4%-6% | 4%-6% | 2%-4% |
| Ex 7474 | 8%-10% | 6%-8% | 4%-6% | 4%-6% | 4%-6% |
| Ex 7475 | 8%-10% | 6%-8% | 4%-6% | 4%-6% | 6%-8% |
| Ex 7476 | 8%-10% | 6%-8% | 4%-6% | 4%-6% | 8%-10% |
| Ex 7477 | 8%-10% | 6%-8% | 4%-6% | 6%-8% | 0%-1% |
| Ex 7478 | 8%-10% | 6%-8% | 4%-6% | 6%-8% | 1%-2% |
| Ex 7479 | 8%-10% | 6%-8% | 4%-6% | 6%-8% | 2%-4% |
| Ex 7480 | 8%-10% | 6%-8% | 4%-6% | 6%-8% | 4%-6% |
| Ex 7481 | 8%-10% | 6%-8% | 4%-6% | 6%-8% | 6%-8% |
| Ex 7482 | 8%-10% | 6%-8% | 4%-6% | 6%-8% | 8%-10% |
| Ex 7483 | 8%-10% | 6%-8% | 4%-6% | 8%-10% | 0%-1% |
| Ex 7484 | 8%-10% | 6%-8% | 4%-6% | 8%-10% | 1%-2% |
| Ex 7485 | 8%-10% | 6%-8% | 4%-6% | 8%-10% | 2%-4% |
| Ex 7486 | 8%-10% | 6%-8% | 4%-6% | 8%-10% | 4%-6% |
| Ex 7487 | 8%-10% | 6%-8% | 4%-6% | 8%-10% | 6%-8% |
| Ex 7488 | 8%-10% | 6%-8% | 4%-6% | 8%-10% | 8%-10% |
| Ex 7489 | 8%-10% | 6%-8% | 6%-8% | 0%-1% | 0%-1% |
| Ex 7490 | 8%-10% | 6%-8% | 6%-8% | 0%-1% | 1%-2% |
| Ex 7491 | 8%-10% | 6%-8% | 6%-8% | 0%-1% | 2%-4% |
| Ex 7492 | 8%-10% | 6%-8% | 6%-8% | 0%-1% | 4%-6% |
| Ex 7493 | 8%-10% | 6%-8% | 6%-8% | 0%-1% | 6%-8% |
| Ex 7494 | 8%-10% | 6%-8% | 6%-8% | 0%-1% | 8%-10% |
| Ex 7495 | 8%-10% | 6%-8% | 6%-8% | 1%-2% | 0%-1% |
| Ex 7496 | 8%-10% | 6%-8% | 6%-8% | 1%-2% | 1%-2% |
| Ex 7497 | 8%-10% | 6%-8% | 6%-8% | 1%-2% | 2%-4% |
| Ex 7498 | 8%-10% | 6%-8% | 6%-8% | 1%-2% | 4%-6% |
| Ex 7499 | 8%-10% | 6%-8% | 6%-8% | 1%-2% | 6%-8% |
| Ex 7500 | 8%-10% | 6%-8% | 6%-8% | 1%-2% | 8%-10% |
| Ex 7501 | 8%-10% | 6%-8% | 6%-8% | 2%-4% | 0%-1% |
| Ex 7502 | 8%-10% | 6%-8% | 6%-8% | 2%-4% | 1%-2% |
| Ex 7503 | 8%-10% | 6%-8% | 6%-8% | 2%-4% | 2%-4% |
| Ex 7504 | 8%-10% | 6%-8% | 6%-8% | 2%-4% | 4%-6% |
| Ex 7505 | 8%-10% | 6%-8% | 6%-8% | 2%-4% | 6%-8% |
| Ex 7506 | 8%-10% | 6%-8% | 6%-8% | 2%-4% | 8%-10% |
| Ex 7507 | 8%-10% | 6%-8% | 6%-8% | 4%-6% | 0%-1% |
| Ex 7508 | 8%-10% | 6%-8% | 6%-8% | 4%-6% | 1%-2% |
| Ex 7509 | 8%-10% | 6%-8% | 6%-8% | 4%-6% | 2%-4% |
| Ex 7510 | 8%-10% | 6%-8% | 6%-8% | 4%-6% | 4%-6% |
| Ex 7511 | 8%-10% | 6%-8% | 6%-8% | 4%-6% | 6%-8% |
| Ex 7512 | 8%-10% | 6%-8% | 6%-8% | 4%-6% | 8%-10% |
| Ex 7513 | 8%-10% | 6%-8% | 6%-8% | 6%-8% | 0%-1% |
| Ex 7514 | 8%-10% | 6%-8% | 6%-8% | 6%-8% | 1%-2% |
| Ex 7515 | 8%-10% | 6%-8% | 6%-8% | 6%-8% | 2%-4% |
| Ex 7516 | 8%-10% | 6%-8% | 6%-8% | 6%-8% | 4%-6% |
| Ex 7517 | 8%-10% | 6%-8% | 6%-8% | 6%-8% | 6%-8% |
| Ex 7518 | 8%-10% | 6%-8% | 6%-8% | 6%-8% | 8%-10% |
| Ex 7519 | 8%-10% | 6%-8% | 6%-8% | 8%-10% | 0%-1% |
| Ex 7520 | 8%-10% | 6%-8% | 6%-8% | 8%-10% | 1%-2% |
| Ex 7521 | 8%-10% | 6%-8% | 6%-8% | 8%-10% | 2%-4% |
| Ex 7522 | 8%-10% | 6%-8% | 6%-8% | 8%-10% | 4%-6% |
| Ex 7523 | 8%-10% | 6%-8% | 6%-8% | 8%-10% | 6%-8% |
| Ex 7524 | 8%-10% | 6%-8% | 6%-8% | 8%-10% | 8%-10% |
| Ex 7525 | 8%-10% | 6%-8% | 8%-10% | 0%-1% | 0%-1% |
| Ex 7526 | 8%-10% | 6%-8% | 8%-10% | 0%-1% | 1%-2% |
| Ex 7527 | 8%-10% | 6%-8% | 8%-10% | 0%-1% | 2%-4% |
| Ex 7528 | 8%-10% | 6%-8% | 8%-10% | 0%-1% | 4%-6% |
| Ex 7529 | 8%-10% | 6%-8% | 8%-10% | 0%-1% | 6%-8% |
| Ex 7530 | 8%-10% | 6%-8% | 8%-10% | 0%-1% | 8%-10% |
| Ex 7531 | 8%-10% | 6%-8% | 8%-10% | 1%-2% | 0%-1% |
| Ex 7532 | 8%-10% | 6%-8% | 8%-10% | 1%-2% | 1%-2% |
| Ex 7533 | 8%-10% | 6%-8% | 8%-10% | 1%-2% | 2%-4% |
| Ex 7534 | 8%-10% | 6%-8% | 8%-10% | 1%-2% | 4%-6% |
| Ex 7535 | 8%-10% | 6%-8% | 8%-10% | 1%-2% | 6%-8% |
| Ex 7536 | 8%-10% | 6%-8% | 8%-10% | 1%-2% | 8%-10% |
| Ex 7537 | 8%-10% | 6%-8% | 8%-10% | 2%-4% | 0%-1% |
| Ex 7538 | 8%-10% | 6%-8% | 8%-10% | 2%-4% | 1%-2% |
| Ex 7539 | 8%-10% | 6%-8% | 8%-10% | 2%-4% | 2%-4% |
| Ex 7540 | 8%-10% | 6%-8% | 8%-10% | 2%-4% | 4%-6% |
| Ex 7541 | 8%-10% | 6%-8% | 8%-10% | 2%-4% | 6%-8% |
| Ex 7542 | 8%-10% | 6%-8% | 8%-10% | 2%-4% | 8%-10% |
| Ex 7543 | 8%-10% | 6%-8% | 8%-10% | 4%-6% | 0%-1% |
| Ex 7544 | 8%-10% | 6%-8% | 8%-10% | 4%-6% | 1%-2% |
| Ex 7545 | 8%-10% | 6%-8% | 8%-10% | 4%-6% | 2%-4% |
| Ex 7546 | 8%-10% | 6%-8% | 8%-10% | 4%-6% | 4%-6% |
| Ex 7547 | 8%-10% | 6%-8% | 8%-10% | 4%-6% | 6%-8% |
| Ex 7548 | 8%-10% | 6%-8% | 8%-10% | 4%-6% | 8%-10% |
| Ex 7549 | 8%-10% | 6%-8% | 8%-10% | 6%-8% | 0%-1% |
| Ex 7550 | 8%-10% | 6%-8% | 8%-10% | 6%-8% | 1%-2% |
| Ex 7551 | 8%-10% | 6%-8% | 8%-10% | 6%-8% | 2%-4% |
| Ex 7552 | 8%-10% | 6%-8% | 8%-10% | 6%-8% | 4%-6% |
| Ex 7553 | 8%-10% | 6%-8% | 8%-10% | 6%-8% | 6%-8% |
| Ex 7554 | 8%-10% | 6%-8% | 8%-10% | 6%-8% | 8%-10% |
| Ex 7555 | 8%-10% | 6%-8% | 8%-10% | 8%-10% | 0%-1% |
| Ex 7556 | 8%-10% | 6%-8% | 8%-10% | 8%-10% | 1%-2% |
| Ex 7557 | 8%-10% | 6%-8% | 8%-10% | 8%-10% | 2%-4% |
| Ex 7558 | 8%-10% | 6%-8% | 8%-10% | 8%-10% | 4%-6% |
| Ex 7559 | 8%-10% | 6%-8% | 8%-10% | 8%-10% | 6%-8% |
| Ex 7560 | 8%-10% | 6%-8% | 8%-10% | 8%-10% | 8%-10% |
| Ex 7561 | 8%-10% | 8%-10% | 0%-1% | 0%-1% | 0%-1% |
| Ex 7562 | 8%-10% | 8%-10% | 0%-1% | 0%-1% | 1%-2% |
| Ex 7563 | 8%-10% | 8%-10% | 0%-1% | 0%-1% | 2%-4% |
| Ex 7564 | 8%-10% | 8%-10% | 0%-1% | 0%-1% | 4%-6% |
| Ex 7565 | 8%-10% | 8%-10% | 0%-1% | 0%-1% | 6%-8% |
| Ex 7566 | 8%-10% | 8%-10% | 0%-1% | 0%-1% | 8%-10% |
| Ex 7567 | 8%-10% | 8%-10% | 0%-1% | 1%-2% | 0%-1% |
| Ex 7568 | 8%-10% | 8%-10% | 0%-1% | 1%-2% | 1%-2% |
| Ex 7569 | 8%-10% | 8%-10% | 0%-1% | 1%-2% | 2%-4% |
| Ex 7570 | 8%-10% | 8%-10% | 0%-1% | 1%-2% | 4%-6% |
| Ex 7571 | 8%-10% | 8%-10% | 0%-1% | 1%-2% | 6%-8% |
| Ex 7572 | 8%-10% | 8%-10% | 0%-1% | 1%-2% | 8%-10% |
| Ex 7573 | 8%-10% | 8%-10% | 0%-1% | 2%-4% | 0%-1% |
| Ex 7574 | 8%-10% | 8%-10% | 0%-1% | 2%-4% | 1%-2% |
| Ex 7575 | 8%-10% | 8%-10% | 0%-1% | 2%-4% | 2%-4% |
| Ex 7576 | 8%-10% | 8%-10% | 0%-1% | 2%-4% | 4%-6% |
| Ex 7577 | 8%-10% | 8%-10% | 0%-1% | 2%-4% | 6%-8% |
| Ex 7578 | 8%-10% | 8%-10% | 0%-1% | 2%-4% | 8%-10% |
| Ex 7579 | 8%-10% | 8%-10% | 0%-1% | 4%-6% | 0%-1% |
| Ex 7580 | 8%-10% | 8%-10% | 0%-1% | 4%-6% | 1%-2% |
| Ex 7581 | 8%-10% | 8%-10% | 0%-1% | 4%-6% | 2%-4% |
| Ex 7582 | 8%-10% | 8%-10% | 0%-1% | 4%-6% | 4%-6% |
| Ex 7583 | 8%-10% | 8%-10% | 0%-1% | 4%-6% | 6%-8% |
| Ex 7584 | 8%-10% | 8%-10% | 0%-1% | 4%-6% | 8%-10% |
| Ex 7585 | 8%-10% | 8%-10% | 0%-1% | 6%-8% | 0%-1% |
| Ex 7586 | 8%-10% | 8%-10% | 0%-1% | 6%-8% | 1%-2% |
| Ex 7587 | 8%-10% | 8%-10% | 0%-1% | 6%-8% | 2%-4% |
| Ex 7588 | 8%-10% | 8%-10% | 0%-1% | 6%-8% | 4%-6% |
| Ex 7589 | 8%-10% | 8%-10% | 0%-1% | 6%-8% | 6%-8% |
| Ex 7590 | 8%-10% | 8%-10% | 0%-1% | 6%-8% | 8%-10% |
| Ex 7591 | 8%-10% | 8%-10% | 0%-1% | 8%-10% | 0%-1% |
| Ex 7592 | 8%-10% | 8%-10% | 0%-1% | 8%-10% | 1%-2% |
| Ex 7593 | 8%-10% | 8%-10% | 0%-1% | 8%-10% | 2%-4% |
| Ex 7594 | 8%-10% | 8%-10% | 0%-1% | 8%-10% | 4%-6% |
| Ex 7595 | 8%-10% | 8%-10% | 0%-1% | 8%-10% | 6%-8% |
| Ex 7596 | 8%-10% | 8%-10% | 0%-1% | 8%-10% | 8%-10% |
| Ex 7597 | 8%-10% | 8%-10% | 1%-2% | 0%-1% | 0%-1% |
| Ex 7598 | 8%-10% | 8%-10% | 1%-2% | 0%-1% | 1%-2% |
| Ex 7599 | 8%-10% | 8%-10% | 1%-2% | 0%-1% | 2%-4% |
| Ex 7600 | 8%-10% | 8%-10% | 1%-2% | 0%-1% | 4%-6% |
| Ex 7601 | 8%-10% | 8%-10% | 1%-2% | 0%-1% | 6%-8% |
| Ex 7602 | 8%-10% | 8%-10% | 1%-2% | 0%-1% | 8%-10% |
| Ex 7603 | 8%-10% | 8%-10% | 1%-2% | 1%-2% | 0%-1% |
| Ex 7604 | 8%-10% | 8%-10% | 1%-2% | 1%-2% | 1%-2% |
| Ex 7605 | 8%-10% | 8%-10% | 1%-2% | 1%-2% | 2%-4% |
| Ex 7606 | 8%-10% | 8%-10% | 1%-2% | 1%-2% | 4%-6% |
| Ex 7607 | 8%-10% | 8%-10% | 1%-2% | 1%-2% | 6%-8% |
| Ex 7608 | 8%-10% | 8%-10% | 1%-2% | 1%-2% | 8%-10% |
| Ex 7609 | 8%-10% | 8%-10% | 1%-2% | 2%-4% | 0%-1% |
| Ex 7610 | 8%-10% | 8%-10% | 1%-2% | 2%-4% | 1%-2% |
| Ex 7611 | 8%-10% | 8%-10% | 1%-2% | 2%-4% | 2%-4% |
| Ex 7612 | 8%-10% | 8%-10% | 1%-2% | 2%-4% | 4%-6% |
| Ex 7613 | 8%-10% | 8%-10% | 1%-2% | 2%-4% | 6%-8% |
| Ex 7614 | 8%-10% | 8%-10% | 1%-2% | 2%-4% | 8%-10% |
| Ex 7615 | 8%-10% | 8%-10% | 1%-2% | 4%-6% | 0%-1% |
| Ex 7616 | 8%-10% | 8%-10% | 1%-2% | 4%-6% | 1%-2% |
| Ex 7617 | 8%-10% | 8%-10% | 1%-2% | 4%-6% | 2%-4% |
| Ex 7618 | 8%-10% | 8%-10% | 1%-2% | 4%-6% | 4%-6% |
| Ex 7619 | 8%-10% | 8%-10% | 1%-2% | 4%-6% | 6%-8% |
| Ex 7620 | 8%-10% | 8%-10% | 1%-2% | 4%-6% | 8%-10% |
| Ex 7621 | 8%-10% | 8%-10% | 1%-2% | 6%-8% | 0%-1% |
| Ex 7622 | 8%-10% | 8%-10% | 1%-2% | 6%-8% | 1%-2% |
| Ex 7623 | 8%-10% | 8%-10% | 1%-2% | 6%-8% | 2%-4% |
| Ex 7624 | 8%-10% | 8%-10% | 1%-2% | 6%-8% | 4%-6% |
| Ex 7625 | 8%-10% | 8%-10% | 1%-2% | 6%-8% | 6%-8% |
| Ex 7626 | 8%-10% | 8%-10% | 1%-2% | 6%-8% | 8%-10% |
| Ex 7627 | 8%-10% | 8%-10% | 1%-2% | 8%-10% | 0%-1% |
| Ex 7628 | 8%-10% | 8%-10% | 1%-2% | 8%-10% | 1%-2% |
| Ex 7629 | 8%-10% | 8%-10% | 1%-2% | 8%-10% | 2%-4% |
| Ex 7630 | 8%-10% | 8%-10% | 1%-2% | 8%-10% | 4%-6% |
| Ex 7631 | 8%-10% | 8%-10% | 1%-2% | 8%-10% | 6%-8% |
| Ex 7632 | 8%-10% | 8%-10% | 1%-2% | 8%-10% | 8%-10% |
| Ex 7633 | 8%-10% | 8%-10% | 2%-4% | 0%-1% | 0%-1% |
| Ex 7634 | 8%-10% | 8%-10% | 2%-4% | 0%-1% | 1%-2% |
| Ex 7635 | 8%-10% | 8%-10% | 2%-4% | 0%-1% | 2%-4% |
| Ex 7636 | 8%-10% | 8%-10% | 2%-4% | 0%-1% | 4%-6% |
| Ex 7637 | 8%-10% | 8%-10% | 2%-4% | 0%-1% | 6%-8% |
| Ex 7638 | 8%-10% | 8%-10% | 2%-4% | 0%-1% | 8%-10% |
| Ex 7639 | 8%-10% | 8%-10% | 2%-4% | 1%-2% | 0%-1% |
| Ex 7640 | 8%-10% | 8%-10% | 2%-4% | 1%-2% | 1%-2% |
| Ex 7641 | 8%-10% | 8%-10% | 2%-4% | 1%-2% | 2%-4% |
| Ex 7642 | 8%-10% | 8%-10% | 2%-4% | 1%-2% | 4%-6% |
| Ex 7643 | 8%-10% | 8%-10% | 2%-4% | 1%-2% | 6%-8% |
| Ex 7644 | 8%-10% | 8%-10% | 2%-4% | 1%-2% | 8%-10% |
| Ex 7645 | 8%-10% | 8%-10% | 2%-4% | 2%-4% | 0%-1% |
| Ex 7646 | 8%-10% | 8%-10% | 2%-4% | 2%-4% | 1%-2% |
| Ex 7647 | 8%-10% | 8%-10% | 2%-4% | 2%-4% | 2%-4% |
| Ex 7648 | 8%-10% | 8%-10% | 2%-4% | 2%-4% | 4%-6% |
| Ex 7649 | 8%-10% | 8%-10% | 2%-4% | 2%-4% | 6%-8% |
| Ex 7650 | 8%-10% | 8%-10% | 2%-4% | 2%-4% | 8%-10% |
| Ex 7651 | 8%-10% | 8%-10% | 2%-4% | 4%-6% | 0%-1% |
| Ex 7652 | 8%-10% | 8%-10% | 2%-4% | 4%-6% | 1%-2% |
| Ex 7653 | 8%-10% | 8%-10% | 2%-4% | 4%-6% | 2%-4% |
| Ex 7654 | 8%-10% | 8%-10% | 2%-4% | 4%-6% | 4%-6% |
| Ex 7655 | 8%-10% | 8%-10% | 2%-4% | 4%-6% | 6%-8% |
| Ex 7656 | 8%-10% | 8%-10% | 2%-4% | 4%-6% | 8%-10% |
| Ex 7657 | 8%-10% | 8%-10% | 2%-4% | 6%-8% | 0%-1% |
| Ex 7658 | 8%-10% | 8%-10% | 2%-4% | 6%-8% | 1%-2% |
| Ex 7659 | 8%-10% | 8%-10% | 2%-4% | 6%-8% | 2%-4% |
| Ex 7660 | 8%-10% | 8%-10% | 2%-4% | 6%-8% | 4%-6% |
| Ex 7661 | 8%-10% | 8%-10% | 2%-4% | 6%-8% | 6%-8% |
| Ex 7662 | 8%-10% | 8%-10% | 2%-4% | 6%-8% | 8%-10% |
| Ex 7663 | 8%-10% | 8%-10% | 2%-4% | 8%-10% | 0%-1% |
| Ex 7664 | 8%-10% | 8%-10% | 2%-4% | 8%-10% | 1%-2% |
| Ex 7665 | 8%-10% | 8%-10% | 2%-4% | 8%-10% | 2%-4% |
| Ex 7666 | 8%-10% | 8%-10% | 2%-4% | 8%-10% | 4%-6% |
| Ex 7667 | 8%-10% | 8%-10% | 2%-4% | 8%-10% | 6%-8% |
| Ex 7668 | 8%-10% | 8%-10% | 2%-4% | 8%-10% | 8%-10% |
| Ex 7669 | 8%-10% | 8%-10% | 4%-6% | 0%-1% | 0%-1% |
| Ex 7670 | 8%-10% | 8%-10% | 4%-6% | 0%-1% | 1%-2% |
| Ex 7671 | 8%-10% | 8%-10% | 4%-6% | 0%-1% | 2%-4% |
| Ex 7672 | 8%-10% | 8%-10% | 4%-6% | 0%-1% | 4%-6% |
| Ex 7673 | 8%-10% | 8%-10% | 4%-6% | 0%-1% | 6%-8% |
| Ex 7674 | 8%-10% | 8%-10% | 4%-6% | 0%-1% | 8%-10% |
| Ex 7675 | 8%-10% | 8%-10% | 4%-6% | 1%-2% | 0%-1% |
| Ex 7676 | 8%-10% | 8%-10% | 4%-6% | 1%-2% | 1%-2% |
| Ex 7677 | 8%-10% | 8%-10% | 4%-6% | 1%-2% | 2%-4% |
| Ex 7678 | 8%-10% | 8%-10% | 4%-6% | 1%-2% | 4%-6% |
| Ex 7679 | 8%-10% | 8%-10% | 4%-6% | 1%-2% | 6%-8% |
| Ex 7680 | 8%-10% | 8%-10% | 4%-6% | 1%-2% | 8%-10% |
| Ex 7681 | 8%-10% | 8%-10% | 4%-6% | 2%-4% | 0%-1% |
| Ex 7682 | 8%-10% | 8%-10% | 4%-6% | 2%-4% | 1%-2% |
| Ex 7683 | 8%-10% | 8%-10% | 4%-6% | 2%-4% | 2%-4% |
| Ex 7684 | 8%-10% | 8%-10% | 4%-6% | 2%-4% | 4%-6% |
| Ex 7685 | 8%-10% | 8%-10% | 4%-6% | 2%-4% | 6%-8% |
| Ex 7686 | 8%-10% | 8%-10% | 4%-6% | 2%-4% | 8%-10% |
| Ex 7687 | 8%-10% | 8%-10% | 4%-6% | 4%-6% | 0%-1% |
| Ex 7688 | 8%-10% | 8%-10% | 4%-6% | 4%-6% | 1%-2% |
| Ex 7689 | 8%-10% | 8%-10% | 4%-6% | 4%-6% | 2%-4% |
| Ex 7690 | 8%-10% | 8%-10% | 4%-6% | 4%-6% | 4%-6% |
| Ex 7691 | 8%-10% | 8%-10% | 4%-6% | 4%-6% | 6%-8% |
| Ex 7692 | 8%-10% | 8%-10% | 4%-6% | 4%-6% | 8%-10% |
| Ex 7693 | 8%-10% | 8%-10% | 4%-6% | 6%-8% | 0%-1% |
| Ex 7694 | 8%-10% | 8%-10% | 4%-6% | 6%-8% | 1%-2% |
| Ex 7695 | 8%-10% | 8%-10% | 4%-6% | 6%-8% | 2%-4% |
| Ex 7696 | 8%-10% | 8%-10% | 4%-6% | 6%-8% | 4%-6% |
| Ex 7697 | 8%-10% | 8%-10% | 4%-6% | 6%-8% | 6%-8% |
| Ex 7698 | 8%-10% | 8%-10% | 4%-6% | 6%-8% | 8%-10% |
| Ex 7699 | 8%-10% | 8%-10% | 4%-6% | 8%-10% | 0%-1% |
| Ex 7700 | 8%-10% | 8%-10% | 4%-6% | 8%-10% | 1%-2% |
| Ex 7701 | 8%-10% | 8%-10% | 4%-6% | 8%-10% | 2%-4% |
| Ex 7702 | 8%-10% | 8%-10% | 4%-6% | 8%-10% | 4%-6% |
| Ex 7703 | 8%-10% | 8%-10% | 4%-6% | 8%-10% | 6%-8% |
| Ex 7704 | 8%-10% | 8%-10% | 4%-6% | 8%-10% | 8%-10% |
| Ex 7705 | 8%-10% | 8%-10% | 6%-8% | 0%-1% | 0%-1% |
| Ex 7706 | 8%-10% | 8%-10% | 6%-8% | 0%-1% | 1%-2% |
| Ex 7707 | 8%-10% | 8%-10% | 6%-8% | 0%-1% | 2%-4% |
| Ex 7708 | 8%-10% | 8%-10% | 6%-8% | 0%-1% | 4%-6% |
| Ex 7709 | 8%-10% | 8%-10% | 6%-8% | 0%-1% | 6%-8% |
| Ex 7710 | 8%-10% | 8%-10% | 6%-8% | 0%-1% | 8%-10% |
| Ex 7711 | 8%-10% | 8%-10% | 6%-8% | 1%-2% | 0%-1% |
| Ex 7712 | 8%-10% | 8%-10% | 6%-8% | 1%-2% | 1%-2% |
| Ex 7713 | 8%-10% | 8%-10% | 6%-8% | 1%-2% | 2%-4% |
| Ex 7714 | 8%-10% | 8%-10% | 6%-8% | 1%-2% | 4%-6% |
| Ex 7715 | 8%-10% | 8%-10% | 6%-8% | 1%-2% | 6%-8% |
| Ex 7716 | 8%-10% | 8%-10% | 6%-8% | 1%-2% | 8%-10% |
| Ex 7717 | 8%-10% | 8%-10% | 6%-8% | 2%-4% | 0%-1% |
| Ex 7718 | 8%-10% | 8%-10% | 6%-8% | 2%-4% | 1%-2% |
| Ex 7719 | 8%-10% | 8%-10% | 6%-8% | 2%-4% | 2%-4% |
| Ex 7720 | 8%-10% | 8%-10% | 6%-8% | 2%-4% | 4%-6% |
| Ex 7721 | 8%-10% | 8%-10% | 6%-8% | 2%-4% | 6%-8% |
| Ex 7722 | 8%-10% | 8%-10% | 6%-8% | 2%-4% | 8%-10% |
| Ex 7723 | 8%-10% | 8%-10% | 6%-8% | 4%-6% | 0%-1% |
| Ex 7724 | 8%-10% | 8%-10% | 6%-8% | 4%-6% | 1%-2% |
| Ex 7725 | 8%-10% | 8%-10% | 6%-8% | 4%-6% | 2%-4% |
| Ex 7726 | 8%-10% | 8%-10% | 6%-8% | 4%-6% | 4%-6% |
| Ex 7727 | 8%-10% | 8%-10% | 6%-8% | 4%-6% | 6%-8% |
| Ex 7728 | 8%-10% | 8%-10% | 6%-8% | 4%-6% | 8%-10% |
| Ex 7729 | 8%-10% | 8%-10% | 6%-8% | 6%-8% | 0%-1% |
| Ex 7730 | 8%-10% | 8%-10% | 6%-8% | 6%-8% | 1%-2% |
| Ex 7731 | 8%-10% | 8%-10% | 6%-8% | 6%-8% | 2%-4% |
| Ex 7732 | 8%-10% | 8%-10% | 6%-8% | 6%-8% | 4%-6% |
| Ex 7733 | 8%-10% | 8%-10% | 6%-8% | 6%-8% | 6%-8% |
| Ex 7734 | 8%-10% | 8%-10% | 6%-8% | 6%-8% | 8%-10% |
| Ex 7735 | 8%-10% | 8%-10% | 6%-8% | 8%-10% | 0%-1% |
| Ex 7736 | 8%-10% | 8%-10% | 6%-8% | 8%-10% | 1%-2% |
| Ex 7737 | 8%-10% | 8%-10% | 6%-8% | 8%-10% | 2%-4% |
| Ex 7738 | 8%-10% | 8%-10% | 6%-8% | 8%-10% | 4%-6% |
| Ex 7739 | 8%-10% | 8%-10% | 6%-8% | 8%-10% | 6%-8% |
| Ex 7740 | 8%-10% | 8%-10% | 6%-8% | 8%-10% | 8%-10% |
| Ex 7741 | 8%-10% | 8%-10% | 8%-10% | 0%-1% | 0%-1% |
| Ex 7742 | 8%-10% | 8%-10% | 8%-10% | 0%-1% | 1%-2% |
| Ex 7743 | 8%-10% | 8%-10% | 8%-10% | 0%-1% | 2%-4% |
| Ex 7744 | 8%-10% | 8%-10% | 8%-10% | 0%-1% | 4%-6% |
| Ex 7745 | 8%-10% | 8%-10% | 8%-10% | 0%-1% | 6%-8% |
| Ex 7746 | 8%-10% | 8%-10% | 8%-10% | 0%-1% | 8%-10% |
| Ex 7747 | 8%-10% | 8%-10% | 8%-10% | 1%-2% | 0%-1% |
| Ex 7748 | 8%-10% | 8%-10% | 8%-10% | 1%-2% | 1%-2% |
| Ex 7749 | 8%-10% | 8%-10% | 8%-10% | 1%-2% | 2%-4% |
| Ex 7750 | 8%-10% | 8%-10% | 8%-10% | 1%-2% | 4%-6% |
| Ex 7751 | 8%-10% | 8%-10% | 8%-10% | 1%-2% | 6%-8% |
| Ex 7752 | 8%-10% | 8%-10% | 8%-10% | 1%-2% | 8%-10% |
| Ex 7753 | 8%-10% | 8%-10% | 8%-10% | 2%-4% | 0%-1% |
| Ex 7754 | 8%-10% | 8%-10% | 8%-10% | 2%-4% | 1%-2% |
| Ex 7755 | 8%-10% | 8%-10% | 8%-10% | 2%-4% | 2%-4% |
| Ex 7756 | 8%-10% | 8%-10% | 8%-10% | 2%-4% | 4%-6% |
| Ex 7757 | 8%-10% | 8%-10% | 8%-10% | 2%-4% | 6%-8% |
| Ex 7758 | 8%-10% | 8%-10% | 8%-10% | 2%-4% | 8%-10% |
| Ex 7759 | 8%-10% | 8%-10% | 8%-10% | 4%-6% | 0%-1% |
| Ex 7760 | 8%-10% | 8%-10% | 8%-10% | 4%-6% | 1%-2% |
| Ex 7761 | 8%-10% | 8%-10% | 8%-10% | 4%-6% | 2%-4% |
| Ex 7762 | 8%-10% | 8%-10% | 8%-10% | 4%-6% | 4%-6% |
| Ex 7763 | 8%-10% | 8%-10% | 8%-10% | 4%-6% | 6%-8% |
| Ex 7764 | 8%-10% | 8%-10% | 8%-10% | 4%-6% | 8%-10% |
| Ex 7765 | 8%-10% | 8%-10% | 8%-10% | 6%-8% | 0%-1% |
| Ex 7766 | 8%-10% | 8%-10% | 8%-10% | 6%-8% | 1%-2% |
| Ex 7767 | 8%-10% | 8%-10% | 8%-10% | 6%-8% | 2%-4% |
| Ex 7768 | 8%-10% | 8%-10% | 8%-10% | 6%-8% | 4%-6% |
| Ex 7769 | 8%-10% | 8%-10% | 8%-10% | 6%-8% | 6%-8% |
| Ex 7770 | 8%-10% | 8%-10% | 8%-10% | 6%-8% | 8%-10% |
| Ex 7771 | 8%-10% | 8%-10% | 8%-10% | 8%-10% | 0%-1% |
| Ex 7772 | 8%-10% | 8%-10% | 8%-10% | 8%-10% | 1%-2% |
| Ex 7773 | 8%-10% | 8%-10% | 8%-10% | 8%-10% | 2%-4% |
| Ex 7774 | 8%-10% | 8%-10% | 8%-10% | 8%-10% | 4%-6% |
| Ex 7775 | 8%-10% | 8%-10% | 8%-10% | 8%-10% | 6%-8% |
| Ex 7776 | 8%-10% | 8%-10% | 8%-10% | 8%-10% | 8%-10% |

I claim:

1. A combination of nanoparticles applied onto a mask filtration system, comprising:
1%-10% graphene;
1%-10% titanium dioxide;
1%-10% zinc oxide;
1%-10% copper oxide;
1%-10% silicon dioxide; and
balance solvent.

2. The combination of nanoparticles applied onto the mask filtration system of claim 1,
comprising:
6-8% graphene, 6-8% titanium dioxide, 6-8% zinc oxide, 6-8% copper oxide, and 6-8% silicon dioxide.

3. The combination of nanoparticles applied onto the mask filtration system of claim 2,
comprising:
8% graphene, 8% titanium dioxide, 8% zinc oxide, 8% copper oxide, and 8% silicon dioxide.

4. The combination of nanoparticles applied onto the mask filtration system of claim 1,
comprising:
6-8% graphene, 6-8% titanium dioxide, 1-2% zinc oxide, 6-8% copper oxide, and 1-2% silicon dioxide.

5. The combination of nanoparticles applied onto the mask filtration system of claim 4,
comprising:
8% graphene, 8% titanium dioxide, 1% zinc oxide, 8% copper oxide, and 1% silicon dioxide.

6. The combination of nanoparticles applied onto the mask filtration system of claim 1,
comprising:
6-8% graphene, 6-8% titanium dioxide, 6-8% zinc oxide, 1-2% copper oxide, and 6-8% silicon dioxide.

7. The combination of nanoparticles applied onto the mask filtration system of claim 6,
comprising:
8% graphene, 8% titanium dioxide, 8% zinc oxide, 2% copper oxide, and 8% silicon dioxide.

8. The combination of nanoparticles applied onto the filtration system of claim 1, comprising:
1-2% graphene, 6-8% titanium dioxide, 6-8% zinc oxide, 6-8% copper oxide, and 1-2% silicon dioxide.

9. The combination of nanoparticles applied onto the mask filtration system of claim 1,
comprising:
1% graphene, 8% titanium dioxide, 8% zinc oxide, 8% copper oxide, and 1% silicon dioxide

10. The combination of nanoparticles applied onto the mask filtration system of claim 1,
wherein:
the mask filtration system filters airborne pollutants comprising PM_{2.5} and aerosolized virus particles selected from the group consisting of: SARS-CoV2, MERS, SARS, Influenza, and the like.

11. A mask filtration system, comprising:
an outer non-woven layer-sized and configured to block big droplets and particles;
a middle melt-blown layer sized and configured for electrostatic filtration;
an inner non-woven layer sized and configured to absorb breathed vapor; and
a plurality of nanoparticles applied onto the outer non-woven layer comprising
1%-10% graphene,
1%-10% titanium dioxide,
1%-10% zinc oxide,
1%-10% copper oxide,
1%-10% silicon dioxide, and
balance solvent.

12. The mask filtration system of claim 11, wherein the middle melt-blown layer has a plurality of nanoparticles comprising:
1%-10% graphene,
1%-10% titanium dioxide,
1%-10% zinc oxide,
1%-10% copper oxide,
1%-10% silicon dioxide, and
balance solvent.

13. The mask filtration system of claim 12, wherein the inner non-woven layer has a plurality of nanoparticles comprising:
1%-10% graphene,
1%-10% titanium dioxide,
1%-10% zinc oxide,
1%-10% copper oxide,
1%-10% silicon dioxide, and
balance solvent.

14. The mask filtration system of claim 13, wherein the mask is sized and configured to be worn by a human.
